SEQUENCE LISTING (375 sequences)

SEQ ID NO: 1 - protein, 3 aa, Homo sapiens
  misc_feature (1)..(3): integrin-binding peptide segment
RGD

SEQ ID NO: 2 - protein, 43 aa, Homo sapiens
  misc_feature (1)..(43): wild-type CD8 Stalk
TTTPAPRPPTPAPTIASQPLSLRPEACRPAAGGAVHTRGLDFA

SEQ ID NO: 3 - protein, 42 aa, Homo sapiens
  misc_feature (1)..(42): wild-type CD28 Stalk
FCKIEVMYPPPYLDNEKSNGTIIHVKGKHLCPSPLFPGPSKP

SEQ ID NO: 4 - protein, 4 aa, Artificial Sequence
  Synthetic Hinge
CPPC

SEQ ID NO: 5 - protein, 5 aa, Artificial Sequence
  Synthetic Hinge
DKTHT

SEQ ID NO: 6 - protein, 15 aa, Artificial Sequence
  Synthetic Hinge
CPEPKSCDTPPPCPR

SEQ ID NO: 7 - protein, 12 aa, Artificial Sequence
  Synthetic Hinge
ELKTPLGDTTHT

SEQ ID NO: 8 - protein, 10 aa, Artificial Sequence
  Synthetic Hinge
KSCDKTHTCP

SEQ ID NO: 9 - protein, 7 aa, Artificial Sequence
  Synthetic Hinge
KCCVDCP

SEQ ID NO: 10 - protein, 7 aa, Artificial Sequence
  Synthetic Hinge
KYGPPCP

SEQ ID NO: 11 - protein, 15 aa, Artificial Sequence
  Synthetic Hinge
EPKSCDKTHTCPPCP

SEQ ID NO: 12 - protein, 12 aa, Artificial Sequence
  Synthetic Hinge
ERKCCVECPPCP

SEQ ID NO: 13 - protein, 17 aa, Artificial Sequence
  Synthetic Hinge
ELKTPLGDTTHTCPRCP

SEQ ID NO: 14 - protein, 12 aa, Artificial Sequence
  Synthetic Hinge
SPNMVPHAHHAQ

SEQ ID NO: 15 - protein, 15 aa, Artificial Sequence
  Synthetic Hinge
EPKSCDKTYTCPPCP

SEQ ID NO: 16 - protein, 45 aa, Artificial Sequence
  Synthetic Hinge
TTTPAPRPPTPAPTIASQPLSLRPEACRPAAGGAVHTRGLDFACD

SEQ ID NO: 17 - protein, 24 aa, Homo sapiens
  misc_feature (1)..(24): CD* alpha Transmembrane domain
IYIWAPLAGTCGVLLLSLVITLYC

SEQ ID NO: 18 - protein, 23 aa, Homo sapiens
  misc_feature (1)..(23): CD8 beta Transmembrane domain
LGLLVAGVLVLLVSLGVAIHLCC

SEQ ID NO: 19 - protein, 25 aa, Homo sapiens
  misc_feature (1)..(25): CD4 Transmembrane domain
ALIVLGGVAGLLLFIGLGIFFCVRC

SEQ ID NO: 20 - protein, 23 aa, Homo sapiens
  misc_feature (1)..(23): CD3 zeta Transmembrane domain
LCYLLDGILFIYGVILTALFLRV

SEQ ID NO: 21 - protein, 27 aa, Homo sapiens
  misc_feature (1)..(27): CD28 Transmembrane domain
FWVLVVVGGVLACYSLLVTVAFIIFWV

SEQ ID NO: 22 - protein, 26 aa, Homo sapiens
  misc_feature (1)..(26): OX40 Transmembrane domain
VAAILGLGLVLGLLGPLAILLALYLL

SEQ ID NO: 23 - protein, 24 aa, Homo sapiens
  misc_feature (1)..(24): CD7 Transmembrane domain
ALPAALAVISFLLGLGLGVACVLA

SEQ ID NO: 24 - protein, 69 aa, Homo sapiens
  misc_feature (1)..(69): CD8a Stalk and Transmembrane domain
TTTPAPRPPTPAPTIASQPLSLRPEACRPAAGGAVHTRGLDFACDIYIWAPLAGTCGVLLLSLVITLYC

SEQ ID NO: 25 - protein, 66 aa, Homo sapiens
  misc_feature (1)..(66): CD28 Stalk and Transmembrane domain
IEVMYPPPYLDNEKSNGTIIHVKGKHLCPSPLFPGPSKPFWVLVVVGGVLACYSLLVTVAFIIFWV

SEQ ID NO: 26 - protein, 163 aa, Homo sapiens
  misc_feature (1)..(163): CD3Z Activating domain isoform 1
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 164 aa, Homo sapiens
  misc_feature (1)..(164): CD3Z Activating domain isoform 2
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 112 aa, Homo sapiens
  misc_feature (1)..(112): CD3Z Activating domain isoform 3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 113 aa, Homo sapiens
  misc_feature (1)..(113): CD3Z Activating domain isoform
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 21 aa, Homo sapiens
  misc_feature (1)..(21): CD3Z Activating domain isoform 4
NQLYNELNLGRREEYDVLDKR

SEQ ID NO: 31 - protein, 22 aa, Homo sapiens
  misc_feature (1)..(22): CD3Z Activating domain isoform 5
EGLYNELQKDKMAEAYSEIGMK

SEQ ID NO: 32 - protein, 21 aa, Homo sapiens
  misc_feature (1)..(21): CD3Z Activating domain isoform 6
DGLYQGLSTATKDTYDALHMQ

SEQ ID NO: 33 - protein, 171 aa, Homo sapiens
  misc_feature (1)..(171): CD3D Activating domain isoform 1
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 34 - protein, 127 aa, Homo sapiens
  misc_feature (1)..(127): CD3D Activating domain isoform 2
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 35 - protein, 21 aa, Homo sapiens
  misc_feature (1)..(21): CD3D Activating domain isoform 3
DQVYQPLRDRDDAQYSHLGGN

SEQ ID NO: 36 - protein, 206 aa, Homo sapiens
  misc_feature (1)..(206): CD3E Activating domain isoform 1
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDMSVATIVIVDICITGGLLLLVYYWSKNRKAKAKPVTRGAGAGGRQRGQNKERPPPVPNPDYEPIRKGQRDLYSGLNQRRI

SEQ ID NO: 37 - protein, 21 aa, Homo sapiens
  misc_feature (1)..(21): CD3E Activating domain isoform 2
NPDYEPIRKGQRDLYSGLNQR

SEQ ID NO: 38 - protein, 182 aa, Homo sapiens
  misc_feature (1)..(182): CD3G Activating domain isoform 1
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 39 - protein, 21 aa, Homo sapiens
  misc_feature (1)..(21): CD3G Activating domain isoform 2
DQLYQPLKDREDDQYSHLQGN

SEQ ID NO: 40 - protein, 226 aa, Homo sapiens
  misc_feature (1)..(226): CD79A Activating domain isoform 1
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 41 - protein, 188 aa, Homo sapiens
  misc_feature (1)..(188): CD79A Activating domain isoform 2
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 42 - protein, 21 aa, Homo sapiens
  misc_feature (1)..(21): CD79A Activating domain isoform 3
ENLYEGLNLDDCSMYEDISRG

SEQ ID NO: 43 - protein, 113 aa, Homo sapiens
  misc_feature (1)..(113): DAP12 Activating domain isoform 1
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 44 - protein, 107 aa, Homo sapiens
  misc_feature (1)..(107): DAP12 Activating domain isoform 2
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQ

SEQ ID NO: 45 - protein, 102 aa, Homo sapiens
  misc_feature (1)..(102): DAP12 Activating domain isoform 3
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 46 - protein, 101 aa, Homo sapiens
  misc_feature (1)..(101): DAP12 Activating domain isoform 4
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 47 - protein, 21 aa, Homo sapiens
  misc_feature (1)..(21): DAP12 Activating domain isoform 5
ESPYQELQGQRSDVYSDLNTQ

SEQ ID NO: 48 - protein, 86 aa, Homo sapiens
  misc_feature (1)..(86): FCERlG Activating domain isoform 1
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 49 - protein, 21 aa, Homo sapiens
  misc_feature (1)..(21): FCERlG Activating domain isoform 2
DGVYTGLSTRNQETYETLKHE

SEQ ID NO: 50 - protein, 20 aa, Homo sapiens
  misc_feature (1)..(20): DAP10 Activating domain
RPRRSPAQDGKVYINMPGRG

SEQ ID NO: 51 - protein, 68 aa, Homo sapiens
  misc_feature (1)..(68): CD28 Activating domain
FWVLVVVGGVLACYSLLVTVAFIIFWVRSKRSRLLHSDYMNMTPRRPGPTRKHYQPYAPPRDFAAYRS

SEQ ID NO: 52 - protein, 619 aa, Homo sapiens
  misc_feature (1)..(619): ZAP70 Activating domain
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 53 - protein, 42 aa, Homo sapiens
  misc_feature (1)..(42): CD137 Co-stimulatory domain
KRGRKKLLYIFKQPFMRPVQTTQEEDGCSCRFPEEEEGGCEL

SEQ ID NO: 54 - protein, 41 aa, Homo sapiens
  misc_feature (1)..(41): CD28 Co-stimulatory domain
RSKRSRLLHSDYMNMTPRRPGPTRKHYQPYAPPRDFAAYRS

SEQ ID NO: 55 - protein, 41 aa, Homo sapiens
  misc_feature (1)..(41): IC Co-stimulatory domain
RSKRSRLLHSDYMNMTPRRPGPTRKHYQAYAAARDFAAYRS

SEQ ID NO: 56 - protein, 35 aa, Homo sapiens
  misc_feature (1)..(35): ICOS Co-stimulatory domain
TKKKYSSSVHDPNGEYMFMRAVNTAKKSRLTDVTL

SEQ ID NO: 57 - protein, 37 aa, Homo sapiens
  misc_feature (1)..(37): OX40 Co-stimulatory domain
RRDQRLPPDAHKPPGGGSFRTPIQEEQADAHSTLAKI

SEQ ID NO: 58 - protein, 49 aa, Homo sapiens
  misc_feature (1)..(49): CD27 Co-stimulatory domain
HQRRKYRSNKGESPVEPAEPCRYSCPREEEGSTIPIQEDYRKPEPACSP

SEQ ID NO: 59 - protein, 114 aa, Homo sapiens
  misc_feature (1)..(114): BLTA Co-stimulatory domain
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 60 - protein, 187 aa, Homo sapiens
  misc_feature (1)..(187): CD30 Co-stimulatory domain
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 61 - protein, 54 aa, Homo sapiens
  misc_feature (1)..(54): GITR Co-stimulatory domain
HIWQLRSQCMWPRETQLLLEVPPSTEDARSCQFPEEERGERSAEEKGRLGDLWV

SEQ ID NO: 62 - protein, 60 aa, Homo sapiens
  misc_feature (1)..(60): HVEM Co-stimulatory domain
CVKRRKPRGDVVKVIVSVQRKRQEAEGEATVIEALQAPPDVTTVAVEETIPSFTGRSPNH

SEQ ID NO: 63 - protein, 15 aa, Artificial Sequence
  Synthetic Linker
GGGGSGGGGSGGGGS

SEQ ID NO: 64 - protein, 30 aa, Artificial Sequence
  Synthetic Linker
GGGGSGGGGSGGGGSGGGGSGGGGSGGGGS

SEQ ID NO: 65 - protein, 14 aa, Artificial Sequence
  Synthetic Linker
GGGGSGGGSGGGGS

SEQ ID NO: 66 - protein, 4 aa, Artificial Sequence
  Synthetic Linker
GGSG

SEQ ID NO: 67 - protein, 5 aa, Artificial Sequence
  Synthetic Linker
GGSGG

SEQ ID NO: 68 - protein, 5 aa, Artificial Sequence
  Synthetic Linker
GSGSG

SEQ ID NO: 69 - protein, 5 aa, Artificial Sequence
  Synthetic Linker
GSGGG

SEQ ID NO: 70 - protein, 5 aa, Artificial Sequence
  Synthetic Linker
GGGSG

SEQ ID NO: 71 - protein, 5 aa, Artificial Sequence
  Synthetic Linker
GSSSG

SEQ ID NO: 72 - protein, 21 aa, Homo sapiens
  misc_feature (1)..(21): CD8 Signal peptide
MALPVTALLLPLALLLHAARP

SEQ ID NO: 73 - protein, 9 aa, Artificial Sequence
  Synthetic HA Epitope
YPYDVPDYA

SEQ ID NO: 74 - protein, 8 aa, Artificial Sequence
  Synthetic FLAG epitope
DYKDDDDK

SEQ ID NO: 75 - protein, 10 aa, Artificial Sequence
  Synthetic c-myc Epitope
EQKLISEEDL

SEQ ID NO: 76 - protein, 5 aa, Artificial Sequence
  Synthetic His5 Affinity
HHHHH

SEQ ID NO: 77 - protein, 6 aa, Artificial Sequence
  Synthetic HisX6 Affinity
HHHHHH

SEQ ID NO: 78 - protein, 8 aa, Artificial Sequence
  Synthetic Strep Tag Affinity
WSHPQFEK

SEQ ID NO: 79 - protein, 5 aa, Artificial Sequence
  Synthetic Affinity tag
RYIRS

SEQ ID NO: 80 - protein, 4 aa, Artificial Sequence
  Synthetic Affinity tag
FHHT

SEQ ID NO: 81 - protein, 17 aa, Artificial Sequence
  Synthetic Affinity tag
WEAAAREACCRECCARA

SEQ ID NO: 82 - protein, 357 aa, Homo sapiens
  misc_feature (1)..(357): EGFR Truncation
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 83 - protein, 21 aa, Artificial Sequence
  Synthetic Cleavage signal
GSGEGRGSLLTCGDVEENPGP

SEQ ID NO: 84 - protein, 368 aa, Artificial Sequence
  Synthetic eTAG IL7RA Ins PPCL (interleukin 7 receptor)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPEINNSSGEMDPILLPPCLTISILSFFSVALLVILACVL

SEQ ID NO: 85 - protein, 232 aa, Artificial Sequence
  Synthetic eTAG IL7RA Ins PPCL (interleukin 7 receptor)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQPEINNSSGEMDPILLPPCLTISILSFFSVALLVILACVL

SEQ ID NO: 86 - protein, 194 aa, Artificial Sequence
  Synthetic Myc Tag LMP1 NC_007605_1
MEQKLISEEDLEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 87 - protein, 174 aa, Artificial Sequence
  Synthetic Myc LMP1 NC_007605_1
MEQKLISEEDLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 88 - protein, 184 aa, Artificial Sequence
  Synthetic LMP1 NC_007605_1
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 89 - protein, 162 aa, Artificial Sequence
  Synthetic LMP1 NC_007605_1
MSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 90 - protein, 363 aa, Artificial Sequence
  Synthetic eTAG CRLF2 transcript variant 1 NM_022148_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGAETPTPPKPKLSKCILISSLAILLMVSLLLLSLW

SEQ ID NO: 91 - protein, 227 aa, Artificial Sequence
  Synthetic eTAG CRLF2 transcript variant 1 NM_022148_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQAETPTPPKPKLSKCILISSLAILLMVSLLLLSLW

SEQ ID NO: 92 - protein, 354 aa, Artificial Sequence
  Synthetic eTAG CSF2RB NM_000395_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTESVLPMWVLALIEIFLTIAVLLAL

SEQ ID NO: 93 - protein, 218 aa, Artificial Sequence
  Synthetic eTAG CSF2RB NM_000395_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTESVLPMWVLALIEIFLTIAVLLAL

SEQ ID NO: 94 - protein, 360 aa, Artificial Sequence
  Synthetic eTAG CSF3R transcript variant 1 NM_000760_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTPEGSELHIILGLFGLLLLLNCLCGTAWLCC

SEQ ID NO: 95 - protein, 224 aa, Artificial Sequence
  Synthetic eTAG CSF3R transcript variant 1 NM_000760_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTPEGSELHIILGLFGLLLLLNCLCGTAWLCC

SEQ ID NO: 96 - protein, 359 aa, Artificial Sequence
  Synthetic eTAG EPOR transcript variant 1 NM_000121_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTPSDLDPCCLTLSLILVVILVLLTVLALLS

SEQ ID NO: 97 - protein, 223 aa, Artificial Sequence
  Synthetic eTAG EPOR transcript variant 1 NM_000121_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTPSDLDPCCLTLSLILVVILVLLTVLALLS

SEQ ID NO: 98 - protein, 368 aa, Artificial Sequence
  Synthetic eTAG GHR transcript variant 1 NM_000163_4
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTLPQMSQFTCCEDFYFPWLLCIIFGIFGLTVMLFVFLFS

SEQ ID NO: 99 - protein, 232 aa, Artificial Sequence
  Synthetic eTAG GHR transcript variant 1 NM_000163_4
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTLPQMSQFTCCEDFYFPWLLCIIFGIFGLTVMLFVFLFS

SEQ ID NO: 100 - protein, 360 aa, Artificial Sequence
  Synthetic eTAG truncated after Fn F523C IL27RA NM_004843_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGHLPDNTLRWKVLPGILCLWGLFLLGCGLSLA

SEQ ID NO: 101 - protein, 224 aa, Artificial Sequence
  Synthetic eTAG truncated after Fn F523C IL27RA NM_004843_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQHLPDNTLRWKVLPGILCLWGLFLLGCGLSLA

SEQ ID NO: 102 - protein, 359 aa, Artificial Sequence
  Synthetic eTAG truncated after Fn S505N MPL NM_005373_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGETATETAWISLVTALHLVLGLNAVLGLLLL

SEQ ID NO: 103 - protein, 223 aa, Artificial Sequence
  Synthetic eTAG truncated after Fn S505N MPL NM_005373_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQETATETAWISLVTALHLVLGLNAVLGLLLL

SEQ ID NO: 104 - protein, 368 aa, Artificial Sequence
  Synthetic eTag  0A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKV

SEQ ID NO: 105 - protein, 369 aa, Artificial Sequence
  Synthetic eTag  1A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVA

SEQ ID NO: 106 - protein, 369 aa, Artificial Sequence
  Synthetic eTag  2A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAA

SEQ ID NO: 107 - protein, 371 aa, Artificial Sequence
  Synthetic eTag  3A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAAA

SEQ ID NO: 108 - protein, 372 aa, Artificial Sequence
  Synthetic eTag  4A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAAAA

SEQ ID NO: 109 - protein, 69 aa, Artificial Sequence
  Synthetic Myc Tag 0A JUN NM_002228_3
MTILGTTFGMVFSLLQVVSGEQKLISEEDLLERIARLEEKVKTLKAQNSELASTANMLREQVAQLKQKV

SEQ ID NO: 110 - protein, 70 aa, Artificial Sequence
  Synthetic Myc Tag 1A JUN NM_002228_3
MTILGTTFGMVFSLLQVVSGEQKLISEEDLLERIARLEEKVKTLKAQNSELASTANMLREQVAQLKQKVA

SEQ ID NO: 111 - protein, 71 aa, Artificial Sequence
  Synthetic Myc Tag 2A JUN NM_002228_3
MTILGTTFGMVFSLLQVVSGEQKLISEEDLLERIARLEEKVKTLKAQNSELASTANMLREQVAQLKQKVAA

SEQ ID NO: 112 - protein, 72 aa, Artificial Sequence
  Synthetic Myc Tag 3A JUN NM_002228_3
MTILGTTFGMVFSLLQVVSGEQKLISEEDLLERIARLEEKVKTLKAQNSELASTANMLREQVAQLKQKVAAA

SEQ ID NO: 113 - protein, 73 aa, Artificial Sequence
  Synthetic Myc Tag 4A JUN NM_002228_3
MTILGTTFGMVFSLLQVVSGEQKLISEEDLLERIARLEEKVKTLKAQNSELASTANMLREQVAQLKQKVAAAA

SEQ ID NO: 114 - protein, 23 aa, Artificial Sequence
  Synthetic CD2 transcript variant 1 NM_001328609_1
LIIGICGGGSLLMVFVALLVFYI

SEQ ID NO: 115 - protein, 21 aa, Artificial Sequence
  Synthetic CD3D transcript variant 1 NM_000732_4
GIIVTDVIATLLLALGVFCFA

SEQ ID NO: 116 - protein, 26 aa, Artificial Sequence
  Synthetic CD3E NM_000733_3
VMSVATIVIVDICITGGLLLLVYYWS

SEQ ID NO: 117 - protein, 21 aa, Artificial Sequence
  Synthetic CD3G NM_000073_2
GFLFAEIVSIFVLAVGVYFIA

SEQ ID NO: 118 - protein, 21 aa, Artificial Sequence
  Synthetic CD3Z CD247 transcript variant 1 NM_198053_2
LCYLLDGILFIYGVILTALFL

SEQ ID NO: 119 - protein, 22 aa, Artificial Sequence
  Synthetic CD4 transcript variant 1 and 2 NM_000616_4
MALIVLGGVAGLLLFIGLGIFF

SEQ ID NO: 120 - protein, 21 aa, Artificial Sequence
  Synthetic CD8A transcript variant 1 NM_001768_6
IYIWAPLAGTCGVLLLSLVIT

SEQ ID NO: 121 - protein, 23 aa, Artificial Sequence
  Synthetic CD8B transcript variant 2 NM_172213_3
LGLLVAGVLVLLVSLGVAIHLCC

SEQ ID NO: 122 - protein, 21 aa, Artificial Sequence
  Synthetic CD27 NM_001242_4
ILVIFSGMFLVFTLAGALFLH

SEQ ID NO: 123 - protein, 27 aa, Artificial Sequence
  Synthetic CD28 transcript variant 1 NM_006139_3
FWVLVVVGGVLACYSLLVTVAFIIFWV

SEQ ID NO: 124 - protein, 22 aa, Artificial Sequence
  Synthetic CD40 transcript variant 1 and 6 NM_001250_5
ALVVIPIIFGILFAILLVLVFI

SEQ ID NO: 125 - protein, 22 aa, Artificial Sequence
  Synthetic CD79A transcript variant 1 NM_001783_3
IITAEGIILLFCAVVPGTLLLF

SEQ ID NO: 126 - protein, 22 aa, Artificial Sequence
  Synthetic CD79B transcript variant 3 NM_001039933_2
GIIMIQTLLIILFIIVPIFLLL

SEQ ID NO: 127 - protein, 21 aa, Artificial Sequence
  Synthetic CRLF2 transcript variant 1 NM_022148_3
FILISSLAILLMVSLLLLSLW

SEQ ID NO: 128 - protein, 21 aa, Artificial Sequence
  Synthetic CRLF2 transcript variant 1 NM_022148_3
CILISSLAILLMVSLLLLSLW

SEQ ID NO: 129 - protein, 26 aa, Artificial Sequence
  Synthetic CSF2RA transcript variant 7 and 8 NM_001161529_1
NLGSVYIYVLLIVGTLVCGIVLGFLF

SEQ ID NO: 130 - protein, 19 aa, Artificial Sequence
  Synthetic CSF2RB NM_000395_2
MWVLALIVIFLTIAVLLAL

SEQ ID NO: 131 - protein, 19 aa, Artificial Sequence
  Synthetic CSF2RB NM_000395_2
MWVLALIEIFLTIAVLLAL

SEQ ID NO: 132 - protein, 23 aa, Artificial Sequence
  Synthetic CSF3R transcript variant 1 NM_000760_3
IILGLFGLLLLLTCLCGTAWLCC

SEQ ID NO: 133 - protein, 23 aa, Artificial Sequence
  Synthetic CSF3R transcript variant 1 NM_000760_3
IILGLFGLLLLLNCLCGTAWLCC

SEQ ID NO: 134 - protein, 23 aa, Artificial Sequence
  Synthetic EPOR transcript variant 1 NM_000121_3
LILTLSLILVVILVLLTVLALLS

SEQ ID NO: 135 - protein, 23 aa, Artificial Sequence
  Synthetic EPOR transcript variant 1 NM_000121_3
CCLTLSLILVVILVLLTVLALLS

SEQ ID NO: 136 - protein, 21 aa, Artificial Sequence
  Synthetic FCER1G NM_004106_1
LCYILDAILFLYGIVLTLLYC

SEQ ID NO: 137 - protein, 23 aa, Artificial Sequence
  Synthetic FCGR2C NM_201563_5
IIVAVVTGIAVAAIVAAVVALIY

SEQ ID NO: 138 - protein, 23 aa, Artificial Sequence
  Synthetic FCGRA2 transcript variant 1 NM_001136219_1
IIVAVVIATAVAAIVAAVVALIY

SEQ ID NO: 139 - protein, 24 aa, Artificial Sequence
  Synthetic GHR transcript variant 1 NM_000163_4
FPWLLIIIFGIFGLTVMLFVFLFS

SEQ ID NO: 140 - protein, 24 aa, Artificial Sequence
  Synthetic GHR transcript variant 1 NM_000163_4
FPWLLCIIFGIFGLTVMLFVFLFS

SEQ ID NO: 141 - protein, 21 aa, Artificial Sequence
  Synthetic ICOS NM_012092.3
FWLPIGCAAFVVVCILGCILI

SEQ ID NO: 142 - protein, 21 aa, Artificial Sequence
  Synthetic IFNAR1 NM_000629_2
IWLIVGICIALFALPFVIYAA

SEQ ID NO: 143 - protein, 21 aa, Artificial Sequence
  Synthetic IFNAR2 transcript variant 1 NM_207585_2
IGGIITVFLIALVLTSTIVTL

SEQ ID NO: 144 - protein, 21 aa, Artificial Sequence
  Synthetic IFNGR1 NM_000416_2
SLWIPVVAALLLFLVLSLVFI

SEQ ID NO: 145 - protein, 21 aa, Artificial Sequence
  Synthetic IFNGR2 transcript variant 1 NM_001329128_1
VILISVGTFSLLSVLAGACFF

SEQ ID NO: 146 - protein, 21 aa, Artificial Sequence
  Synthetic IFNLR1 NM_170743_3
FLVLPSLLILLLVIAAGGVIW

SEQ ID NO: 147 - protein, 23 aa, Artificial Sequence
  Synthetic IL1R1 transcript variant 2 NM_001288706_1
HMIGICVTLTVIIVCSVFIYKIF

SEQ ID NO: 148 - protein, 21 aa, Artificial Sequence
  Synthetic IL1RAP transcript variant 1 NM_002182_3
VLLVVILIVVYHVYWLEMVLF

SEQ ID NO: 149 - protein, 21 aa, Artificial Sequence
  Synthetic IL1RL1 transcript variant 1 NM_016232.4
IYCIIAVCSVFLMLINVLVII

SEQ ID NO: 150 - protein, 21 aa, Artificial Sequence
  Synthetic IL1RL2 NM_003854.2
AYLIGGLIALVAVAVSVVYIY

SEQ ID NO: 151 - protein, 19 aa, Artificial Sequence
  Synthetic IL2RA transcript variant 1 NM_000417_2
VAVAGCVFLLISVLLLSGL

SEQ ID NO: 152 - protein, 25 aa, Artificial Sequence
  Synthetic IL2RB transcript variant 1 NM_000878_4
IPWLGHLLVGLSGAFGFIILVYLLI

SEQ ID NO: 153 - protein, 21 aa, Artificial Sequence
  Synthetic IL2RG NM_000206_2
VVISVGSMGLIISLLCVYFWL

SEQ ID NO: 154 - protein, 20 aa, Artificial Sequence
  Synthetic IL3RA transcript variant 1 and 2 NM_002183_3
TSLLIALGTLLALVCVFVIC

SEQ ID NO: 155 - protein, 24 aa, Artificial Sequence
  Synthetic IL4R transcript variant 1 NM_000418_3
LLLGVSVSCIVILAVCLLCYVSIT

SEQ ID NO: 156 - protein, 20 aa, Artificial Sequence
  Synthetic IL5RA transcript variant 1 NM_000564_4
FVIVIMATICFILLILSLIC

SEQ ID NO: 157 - protein, 21 aa, Artificial Sequence
  Synthetic IL6R transcript variant 1 NM_000565_3
TFLVAGGSLAFGTLLCIAIVL

SEQ ID NO: 158 - protein, 22 aa, Artificial Sequence
  Synthetic IL6ST transcript variant 1 and 3 NM_002184_3
AIVVPVCLAFLLTTLLGVLFCF

SEQ ID NO: 159 - protein, 23 aa, Artificial Sequence
  Synthetic IL7RA NM_002185_3
ILLTISILSFFSVALLVILACVL

SEQ ID NO: 160 - protein, 27 aa, Artificial Sequence
  Synthetic IL7RA Ins PPCL (interleukin 7 receptor)
ILLPPCLTISILSFFSVALLVILACVL

SEQ ID NO: 161 - protein, 21 aa, Artificial Sequence
  Synthetic IL9R transcript variant 1 NM_002186_2
GNTLVAVSIFLLLTGPTYLLF

SEQ ID NO: 162 - protein, 21 aa, Artificial Sequence
  Synthetic IL10RA transcript variant 1 NM_001558_3
VIIFFAFVLLLSGALAYCLAL

SEQ ID NO: 163 - protein, 22 aa, Artificial Sequence
  Synthetic IL10RB NM_000628_4
WMVAVILMASVFMVCLALLGCF

SEQ ID NO: 164 - protein, 21 aa, Artificial Sequence
  Synthetic IL11RA NM_001142784_2
SLGILSFLGLVAGALALGLWL

SEQ ID NO: 165 - protein, 25 aa, Artificial Sequence
  Synthetic IL12RB1 transcript variant 1 and 4 NM_005535_2
WLIFFASLGSFLSILLVGVLGYLGL

SEQ ID NO: 166 - protein, 21 aa, Artificial Sequence
  Synthetic IL12RB2 transcript variant 1 and 3 NM_001559_2
WMAFVAPSICIAIIMVGIFST

SEQ ID NO: 167 - protein, 24 aa, Artificial Sequence
  Synthetic IL13RA1 NM_001560_2
LYITMLLIVPVIVAGAIIVLLLYL

SEQ ID NO: 168 - protein, 20 aa, Artificial Sequence
  Synthetic IL13RA2 NM_000640_2
FWLPFGFILILVIFVTGLLL

SEQ ID NO: 169 - protein, 23 aa, Artificial Sequence
  Synthetic IL15RA transcript variant 4 NM_001256765_1
VAISTSTVLLCGLSAVSLLACYL

SEQ ID NO: 170 - protein, 21 aa, Artificial Sequence
  Synthetic IL17RA NM_014339_6
VYWFITGISILLVGSVILLIV

SEQ ID NO: 171 - protein, 21 aa, Artificial Sequence
  Synthetic IL17RB NM_018725_3
LLLLSLLVATWVLVAGIYLMW

SEQ ID NO: 172 - protein, 21 aa, Artificial Sequence
  Synthetic IL17RC transcript variant 1 NM_153460_3
WALVWLACLLFAAALSLILLL

SEQ ID NO: 173 - protein, 21 aa, Artificial Sequence
  Synthetic IL17RD transcript variant 2 NM_017563_4
AVAITVPLVVISAFATLFTVM

SEQ ID NO: 174 - protein, 21 aa, Artificial Sequence
  Synthetic IL17RE transcript variant 1 NM_153480_1
LGLLILALLALLTLLGVVLAL

SEQ ID NO: 175 - protein, 21 aa, Artificial Sequence
  Synthetic IL18R1 transcript variant 1 NM_003855_3
GMIIAVLILVAVVCLVTVCVI

SEQ ID NO: 176 - protein, 21 aa, Artificial Sequence
  Synthetic IL18RAP NM_003853_3
GVVLLYILLGTIGTLVAVLAA

SEQ ID NO: 177 - protein, 21 aa, Artificial Sequence
  Synthetic IL20RA transcript variant 1 NM_014432_3
IIFWYVLPISITVFLFSVMGY

SEQ ID NO: 178 - protein, 21 aa, Artificial Sequence
  Synthetic IL20RB NM_144717_3
VLALFAFVGFMLILVVVPLFV

SEQ ID NO: 179 - protein, 21 aa, Artificial Sequence
  Synthetic IL21R transcript variant 2 NM_181078_2
GWNPHLLLLLLLVIVFIPAFW

SEQ ID NO: 180 - protein, 21 aa, Artificial Sequence
  Synthetic IL22RA1 NM_021258_3
YSFSGAFLFSMGFLVAVLCYL

SEQ ID NO: 181 - protein, 21 aa, Artificial Sequence
  Synthetic IL23R NM_144701_2
LLLGMIVFAVMLSILSLIGIF

SEQ ID NO: 182 - protein, 21 aa, Artificial Sequence
  Synthetic IL27RA NM_004843_3
VLPGILFLWGLFLLGCGLSLA

SEQ ID NO: 183 - protein, 21 aa, Artificial Sequence
  Synthetic IL27RA NM_004843_3
VLPGILCLWGLFLLGCGLSLA

SEQ ID NO: 184 - protein, 24 aa, Artificial Sequence
  Synthetic IL31RA transcript variant 1 NM_139017_5
IILITSLIGGGLLILIILTVAYGL

SEQ ID NO: 185 - protein, 23 aa, Artificial Sequence
  Synthetic LEPR transcript variant 1 NM_002303_5
AGLYVIVPVIISSSILLLGTLLI

SEQ ID NO: 186 - protein, 25 aa, Artificial Sequence
  Synthetic LIFR NM_001127671_1
VGLIIAILIPVAVAVIVGVVTSILC

SEQ ID NO: 187 - protein, 22 aa, Artificial Sequence
  Synthetic MPL NM_005373_2
ISLVTALHLVLGLSAVLGLLLL

SEQ ID NO: 188 - protein, 22 aa, Artificial Sequence
  Synthetic MPL NM_005373_2
ISLVTALHLVLGLNAVLGLLLL

SEQ ID NO: 189 - protein, 21 aa, Artificial Sequence
  Synthetic OSMR transcript variant 4 NM_001323505_1
LIHILLPMVFCVLLIMVMCYL

SEQ ID NO: 190 - protein, 24 aa, Artificial Sequence
  Synthetic PRLR transcript variant 1 NM_000949_6
TTVWISVAVLSAVICLIIVWAVAL

SEQ ID NO: 191 - protein, 21 aa, Artificial Sequence
  Synthetic TNFRSF4 NM_003327_3
VAAILGLGLVLGLLGPLAILL

SEQ ID NO: 192 - protein, 28 aa, Artificial Sequence
  Synthetic TNFRSF8 transcript variant 1 NM_001243_4
PVLDAGPVLFWVILVLVVVVGSSAFLLC

SEQ ID NO: 193 - protein, 27 aa, Artificial Sequence
  Synthetic TNFRSF9 NM_001561_5
IISFFLALTSTALLFLLFFLTLRFSVV

SEQ ID NO: 194 - protein, 21 aa, Artificial Sequence
  Synthetic TNFRSF14 transcript variant 1 NM_003820_3
WWFLSGSLVIVIVCSTVGLII

SEQ ID NO: 195 - protein, 21 aa, Artificial Sequence
  Synthetic TNFRSF18 transcript variant 1 NM_004195_2
LGWLTVVLLAVAACVLLLTSA

SEQ ID NO: 196 - protein, 117 aa, Artificial Sequence
  Synthetic CD2 transcript variant 1 NM_001328609_1
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 197 - protein, 45 aa, Artificial Sequence
  Synthetic CD3D transcript variant 1 NM_000732_4
GHETGRLSGAADTQALLRNDQVYQPLRDRDDAQYSHLGGNWARNK

SEQ ID NO: 198 - protein, 55 aa, Artificial Sequence
  Synthetic CD3E NM_000733_3
KNRKAKAKPVTRGAGAGGRQRGQNKERPPPVPNPDYEPIRKGQRDLYSGLNQRRI

SEQ ID NO: 199 - protein, 45 aa, Artificial Sequence
  Synthetic CD3G NM_000073_2
GQDGVRQSRASDKQTLLPNDQLYQPLKDREDDQYSHLQGNQLRRN

SEQ ID NO: 200 - protein, 40 aa, Artificial Sequence
  Synthetic CD4 transcript variant 1 and 2 NM_000616_4
CVRCRHRRRQAERMSQIKRLLSEKKTCQCPHRFQKTCSPI

SEQ ID NO: 201 - protein, 32 aa, Artificial Sequence
  Synthetic CD8A transcript variant 1 NM_001768_6
LYCNHRNRRRVCKCPRPVVKSGDKPSLSARYV

SEQ ID NO: 202 - protein, 48 aa, Artificial Sequence
  Synthetic CD8B transcript variant 2 NM_172213_3
RRRRARLRFMKQPQGEGISGTFVPQCLHGYYSNTTTSQKLLNPWILKT

SEQ ID NO: 203 - protein, 26 aa, Artificial Sequence
  Synthetic CD8B transcript variant 3 NM_172101_3
RRRRARLRFMKQLRLHPLEKCSRMDY

SEQ ID NO: 204 - protein, 15 aa, Artificial Sequence
  Synthetic CD8B transcript variant 5 NM_004931_4
RRRRARLRFMKQFYK

SEQ ID NO: 205 - protein, 48 aa, Artificial Sequence
  Synthetic CD27 NM_001242_4
QRRKYRSNKGESPVEPAEPCRYSCPREEEGSTIPIQEDYRKPEPACSP

SEQ ID NO: 206 - protein, 41 aa, Artificial Sequence; identical to SEQ ID NO: 55
  Synthetic mutated Delta Lck CD28 transcript variant 1 NM_006139_3

SEQ ID NO: 207 - protein, 41 aa, Artificial Sequence; identical to SEQ ID NO: 54
  Synthetic CD28 transcript variant 1 NM_006139_3

SEQ ID NO: 208 - protein, 62 aa, Artificial Sequence
  Synthetic CD40 transcript variant 1 and 6 NM_001250_5
KKVAKKPTNKAPHPKQEPQEINFPDDLPGSNTAAPVQETLHGCQPVTQEDGKESRISVQERQ

SEQ ID NO: 209 - protein, 66 aa, Artificial Sequence
  Synthetic CD40 transcript variant 5 NM_001322421_1
SESSEKVAKKPTNKAPHPKQEPQEINFPDDLPGSNTAAPVQETLHGCQPVTQEDGKESRISVQERQ

SEQ ID NO: 210 - protein, 61 aa, Artificial Sequence
  Synthetic CD79A transcript variant 1 NM_001783_3
RKRWQNEKLGLDAGDEYEDENLYEGLNLDDCSMYEDISRGLQGTYQDVGSLNIGDVQLEKP

SEQ ID NO: 211 - protein, 49 aa, Artificial Sequence
  Synthetic CD79B transcript variant 3 NM_001039933_2
LDKDDSKAGMEEDHTYEGLDIDQTATYEDIVTLRTGEVKWSVGEHPGQE

SEQ ID NO: 212 - protein, 119 aa, Artificial Sequence
  Synthetic CRLF2 transcript variant 1 NM_022148_3
KLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 213 - protein, 437 aa, Artificial Sequence
  Synthetic CSF2RB NM_000395_2
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 214 - protein, 54 aa, Artificial Sequence
  Synthetic CSF2RA transcript variant 7 and 8 NM_001161529_1
KRFLRIQRLFPPVPQIKDKLNDNHEVEDEIIWEEFTPEEGKGYREEVLTVKEIT

SEQ ID NO: 215 - protein, 64 aa, Artificial Sequence
  Synthetic CSF2RA transcript variant 9 NM_001161531_1
KRFLRIQRLFPPVPQIKDKLNDNHEVEDEMGPQRHHRCGWNLYPTPGPSPGSGSSPRLGSESSL

SEQ ID NO: 216 - protein, 186 aa, Artificial Sequence
  Synthetic CSF3R transcript variant 1 NM_000760_3
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 217 - protein, 213 aa, Artificial Sequence
  Synthetic CSF3R transcript variant 3 NM_156039_3
SPNRKNPLWPSVPDPAHSSLGSWVPTIMEELPGPRQGQWLGQTSEMSRALTPHPCVQDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 218 - protein, 133 aa, Artificial Sequence
  Synthetic CSF3R transcript variant 4 NM_172313_2
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQAGPPRRSAYFKDQIMLHPAPPNGLLCLFPITSVL

SEQ ID NO: 219 - protein, 235 aa, Artificial Sequence
  Synthetic EPOR transcript variant 1 NM_000121_3
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 220 - protein, 235 aa, Artificial Sequence
  Synthetic EPOR transcript variant 1 NM_000121_3
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKFLFLVVSDSGISTDYSSGDSQGAQGGLSDGPYSNPYENSLIPAAEPLPPSYVACS

SEQ ID NO: 221 - protein, 42 aa, Artificial Sequence
  Synthetic FCER1G NM_004106_1
RLKIQVRKAAITSYEKSDGVYTGLSTRNQETYETLKHEKPPQ

SEQ ID NO: 222 - protein, 77 aa, Artificial Sequence
  Synthetic FCGR2C NM_201563_5
CRKKRISANSTDPVKAAQFEPPGRQMIAIRKRQPEETNNDYETADGGYMTLNPRAPTDDDKNIYLTLPPNDHVNSNN

SEQ ID NO: 223 - protein, 77 aa, Artificial Sequence
  Synthetic FCGRA2 transcript variant 1 NM_001136219_1
CRKKRISANSTDPVKAAQFEPPGRQMIAIRKRQLEETNNDYETADGGYMTLNPRAPTDDDKNIYLTLPPNDHVNSNN

SEQ ID NO: 224 - protein, 350 aa, Artificial Sequence
  Synthetic GHR transcript variant 1 NM_000163_4
KQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 225 - protein, 38 aa, Artificial Sequence
  Synthetic ICOS NM_012092.3
CWLTKKKYSSSVHDPNGEYMFMRAVNTAKKSRLTDVTL

SEQ ID NO: 226 - protein, 100 aa, Artificial Sequence
  Synthetic IFNAR1 NM_000629_2
KVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 227 - protein, 251 aa, Artificial Sequence
  Synthetic IFNAR2 transcript variant 1 NM_207585_2
KWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 228 - protein, 67 aa, Artificial Sequence
  Synthetic IFNAR2 transcript variant 2 NM_000874_4
KWIGYICLRNSLPKVLRQGLAKGWNAVAIHRCSHNALQSETPELKQSSCLSFPSSWDYKRASLCPSD

SEQ ID NO: 229 - protein, 223 aa, Artificial Sequence
  Synthetic IFNGR1 NM_000416_2
CFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 230 - protein, 69 aa, Artificial Sequence
  Synthetic IFNGR2 transcript variant 1 NM_001329128_1
LVLKYRGLIKYWFHTPPSIPLQIEEYLKDPTQPILEALDKDSSPKDDVWDSVSIISFPEKEQEDVLQTL

SEQ ID NO: 231 - protein, 271 aa, Artificial Sequence
  Synthetic IFNLR1 NM_170743_3
KTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 232 - protein, 242 aa, Artificial Sequence
  Synthetic IFNLR1 transcript variant 2 NM_173064_2
KTLMGNPWFQRAKMPRALELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 233 - protein, 179 aa, Artificial Sequence
  Synthetic IL1R1 transcript variant 2 NM_001288706_1
KIDIVLWYRDSCYDFLPIKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 234 - protein, 210 aa, Artificial Sequence
  Synthetic IL1R1 transcript variant 3 NM_001320978_1
KIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 235 - protein, 182 aa, Artificial Sequence
  Synthetic IL1RAP transcript variant 1 NM_002182_3
YRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 236 - protein, 299 aa, Artificial Sequence
  Synthetic IL1RAP transcript variant 6 NM_001167931_1
YRAHFGTDETILDGKEYDIYVSYARNAEEEEFVLLTLRGVLENEFGYKLCIFDRDSLPGGNTVEAVFDFIQRSRRMIVVLSPDYVTEKSISMLEFKLGVMCQNSIATKLIVVEYRPLEHPHPGILQLKESVSFVSWKGEKSKHSGSKFWKALRLALPLRSLSASSGWNESCSSQSDISLDHVQRRRSRLKEPPELQSSERAAGSPPAPGTMSKHRGKSSATCRCCVTYCEGENHLRNKSRAEIHNQPQWETHLCKPVPQESETQWIQNGTRLEPPAPQISALALHHFTDLSNNNDFYIL

SEQ ID NO: 237 - protein, 207 aa, Artificial Sequence
  Synthetic IL1RL1 transcript variant 1 NM_016232.4
LKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 238 - protein, 219 aa, Artificial Sequence
  Synthetic IL1RL2 NM_003854.2
NIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 239 - protein, 13 aa, Artificial Sequence
  Synthetic IL2RA transcript variant 1 NM_000417_2
TWQRRQRKSRRTI

SEQ ID NO: 240 - protein, 286 aa, Artificial Sequence
  Synthetic IL2RB transcript variant 1 NM_000878_4
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 241 - protein, 86 aa, Artificial Sequence
  Synthetic IL2RG NM_000206_2
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 242 - protein, 53 aa, Artificial Sequence
  Synthetic IL3RA transcript variant 1 and 2 NM_002183_3
RRYLVMQRLFPRIPHMKDPIGDSFQNDKLVVWEAGKAGLEECLVTEVQVVQKT

SEQ ID NO: 243 - protein, 569 aa, Artificial Sequence
  Synthetic IL4R transcript variant 1 NM_000418_3
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 244 - protein, 569 aa, Artificial Sequence; identical to SEQ ID NO: 243
  Synthetic IL4R transcript variant 1 NM_000418_3

SEQ ID NO: 245 - protein, 58 aa, Artificial Sequence
  Synthetic IL5RA transcript variant 1 NM_000564_4
KICHLWIKLFPPIPAPKSNIKDLFVTTNYEKAGSSETEIEVICYIEKPGVETLEDSVF

SEQ ID NO: 246 - protein, 82 aa, Artificial Sequence
  Synthetic IL6R transcript variant 1 NM_000565_3
RFKKTWKLRALKEGKTSMHPPYSLGQLVPERPRPTPVLVPLISPPVSPSSLGSDNTSSHNRPDARDPRSPYDISNTDYFFPR

SEQ ID NO: 247 - protein, 277 aa, Artificial Sequence
  Synthetic IL6ST transcript variant 1 and 3 NM_002184_3
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 248 - protein, 196 aa, Artificial Sequence
  Synthetic IL7RA Isoform 1 NM_002185.4
WKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 249 - protein, 35 aa, Artificial Sequence
  Synthetic IL7RA Isoform 3 (C-term deletion) (interleukin 7 receptor)
WKKRIKPIVWPSLPDHKKTLEHLCKKPRKVSVFGA

SEQ ID NO: 250 - protein, 230 aa, Artificial Sequence
  Synthetic IL9R transcript variant 1 NM_002186_2
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 251 - protein, 322 aa, Artificial Sequence
  Synthetic IL10RA transcript variant 1 NM_001558_3
QLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 252 - protein, 83 aa, Artificial Sequence
  Synthetic IL10RB NM_000628_4
ALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 253 - protein, 31 aa, Artificial Sequence
  Synthetic IL11RA NM_001142784_2
RLRRGGKDGSPKPGFLASVIPVDRRPGAPNL

SEQ ID NO: 254 - protein, 92 aa, Artificial Sequence
  Synthetic IL12RB1 transcript variant 1 and 4 NM_005535_2
NRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 255 - protein, 90 aa, Artificial Sequence
  Synthetic IL12RB1 transcript variant 3 NM_001290023_1
NRAARHLCPPLPTPCASSAIEFPGGKETWQWINPVDFQEEASLQEALVVEMSWDKGERTEPLEKTELPEGAPELALDTELSLEDGDRCDR

SEQ ID NO: 256 - protein, 219 aa, Artificial Sequence
  Synthetic IL12RB2 transcript variant 1 and 3 NM_001559_2
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 257 - protein, 60 aa, Artificial Sequence
  Synthetic IL13RA1 NM_001560_2
KRLKIIIFPPIPDPGKIFKEMFGDQNDDTLHWKKYDIYEKQTKEETDSVVLIENLKKASQ

SEQ ID NO: 258 - protein, 17 aa, Artificial Sequence
  Synthetic IL13RA2 NM_000640_2
RKPNTYPKMIPEFFCDT

SEQ ID NO: 259 - protein, 39 aa, Artificial Sequence
  Synthetic IL15RA transcript variant 4 NM_001256765_1
KSRQTPPLASVEMEAMEALPVTWGTSSRDEDLENCSHHL

SEQ ID NO: 260 - protein, 525 aa, Artificial Sequence
  Synthetic IL17RA NM_014339_6
CMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 261 - protein, 189 aa, Artificial Sequence
  Synthetic IL17RB NM_018725_3
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 262 - protein, 232 aa, Artificial Sequence
  Synthetic IL17RC transcript variant 1 NM_153460_3
KKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 263 - protein, 219 aa, Artificial Sequence
  Synthetic IL17RC transcript variant 4 NM_001203263_1
KKDHAKAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 264 - protein, 419 aa, Artificial Sequence
  Synthetic IL17RD transcript variant 2 NM_017563_4
CRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 265 - protein, 192 aa, Artificial Sequence
  Synthetic IL17RE transcript variant 1 NM_153480_1
TCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 266 - protein, 191 aa, Artificial Sequence
  Synthetic IL18R1 transcript variant 1 NM_003855_3
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 267 - protein, 222 aa, Artificial Sequence
  Synthetic IL18RAP NM_003853_3
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 268 - protein, 282 aa, Artificial Sequence
  Synthetic IL20RA transcript variant 1 NM_014432_3
SIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSLTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 269 - protein, 57 aa, Artificial Sequence
  Synthetic IL20RB NM_144717_3
WKMGRLLQYSCCPVVVLPDTLKITNSPQKLISCRREEVDACATAVMSPEELLRAWIS

SEQ ID NO: 270 - protein, 285 aa, Artificial Sequence
  Synthetic IL21R transcript variant 2 NM_181078_2
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 271 - protein, 325 aa, Artificial Sequence
  Synthetic IL22RA1 NM_021258_3
SYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 272 - protein, 253 aa, Artificial Sequence
  Synthetic IL23R NM_144701_2
NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 273 - protein, 99 aa, Artificial Sequence
  Synthetic IL27RA NM_004843_3
TSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 274 - protein, 86 aa, Artificial Sequence
  Synthetic IL27RA NM_004843_3
TSWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 275 - protein, 189 aa, Artificial Sequence
  Synthetic IL31RA transcript variant 1 NM_139017_5
KKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV

SEQ ID NO: 276 - protein, 106 aa, Artificial Sequence
  Synthetic IL31RA transcript variant 4 NM_001242638_1
KKPNKLTHLCWPTVPNPAESSIATWHGDDFKDKLNLKESDDSVNTEDRILKPCSTPSDKLVIDKLVVNFGNVLQEIFTDEARTGQENNLGGEKNGTRILSSCPTSI

SEQ ID NO: 277 - protein, 303 aa, Artificial Sequence
  Synthetic LEPR transcript variant 1 NM_002303_5
SHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 278 - protein, 96 aa, Artificial Sequence
  Synthetic LEPR transcript variant 2 NM_001003680_3
SHQRMKKLFWEDVPNPKNCSWAQGLNFQKMLEGSMFVKSHHHSLISSTQGHKHCGRPQGPLHRKTRDLCSLVYLLTLPPLLSYDPAKSPSVRNTQE

SEQ ID NO: 279 - protein, 34 aa, Artificial Sequence
  Synthetic LEPR transcript variant 3 NM_001003679_3
SHQRMKKLFWEDVPNPKNCSWAQGLNFQKRTDIL

SEQ ID NO: 280 - protein, 44 aa, Artificial Sequence
  Synthetic LEPR transcript variant 5 NM_001198688_1
SHQRMKKLFWEDVPNPKNCSWAQGLNFQKKMPGTKELLGGGWLT

SEQ ID NO: 281 - protein, 239 aa, Artificial Sequence
  Synthetic LIFR NM_001127671_1
YRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 282 - protein, 202 aa, Artificial Sequence
  Synthetic LMP1 NC_007605_1
YYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 283 - protein, 122 aa, Artificial Sequence
  Synthetic MPL NM_005373_2
RWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 284 - protein, 304 aa, Artificial Sequence
  Synthetic MYD88 transcript variant 1 NM_001172567_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRLARRPRGGCRRMVVVVSDDYLQSKECDFQTKFALSLSPGAHQKRLIPIKYKAMKKEFPSILRFITVCDYTNPCTKSWFWTRLAKALSLP

SEQ ID NO: 285 - protein, 296 aa, Artificial Sequence
  Synthetic MYD88 transcript variant 2 NM_002468_4
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 286 - protein, 251 aa, Artificial Sequence
  Synthetic MYD88 transcript variant 3 NM_001172568_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 287 - protein, 191 aa, Artificial Sequence
  Synthetic MYD88 transcript variant 4 NM_001172569_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGAAGWWWLSLMITCRARNVTSRPNLHSASLQVPIRSD

SEQ ID NO: 288 - protein, 146 aa, Artificial Sequence
  Synthetic MYD88 transcript variant 5 NM_001172566_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGAAGWWWLSLMITCRARNVTSRPNLHSASLQVPIRSD

SEQ ID NO: 289 - protein, 172 aa, Artificial Sequence
  Synthetic MYD88 transcript variant 1 NM_001172567_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 290 - protein, 127 aa, Artificial Sequence
  Synthetic MYD88 transcript variant 3 NM_001172568_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDI

SEQ ID NO: 291 - protein, 304 aa, Artificial Sequence
  Synthetic MYD88 transcript variant 1 NM_001172567_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRLARRPRGGCRRMVVVVSDDYLQSKECDFQTKFALSLSPGAHQKRPIPIKYKAMKKEFPSILRFITVCDYTNPCTKSWFWTRLAKALSLP

SEQ ID NO: 292 - protein, 296 aa, Artificial Sequence
  Synthetic MYD88 transcript variant 2 NM_002468_4
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRPIPIKYKAMKKEFPSILRFITVCDYTNPCTKSWFWTRLAKALSLP

SEQ ID NO: 293 - protein, 251 aa, Artificial Sequence
  Synthetic MYD88 transcript variant 3 NM_001172568_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRPIPIKYKAMKKEFPSILRFITVCDYTNPCTKSWFWTRLAKALSLP

SEQ ID NO: 294 - protein, 218 aa, Artificial Sequence
  Synthetic OSMR transcript variant 4 NM_001323505_1
KSQWIKETCYPDIPDPYKSSILSLIKFKENPHLIIMNVSDCIPDAIEVVSKPEGTKIQFLGTRKSLTETELTKPNYLYLLPTEKNHSGPGPCICFENLTYNQAASDSGSCGHVPVSPKAPSMLGLMTSPENVLKALEKNYMNSLGEIPAGETSLNYVSQLASPMFGDKDSLPTNPVEAPHCSEYKMQMAVSLRLALPPPTENSSLSSITLLDPGEHYC

SEQ ID NO: 295 - protein, 364 aa, Artificial Sequence
  Synthetic PRLR transcript variant 1 NM_000949_6
KGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 296 - protein, 42 aa, Artificial Sequence
  Synthetic TNFRSF4 NM_003327_3
ALYLLRRDQRLPPDAHKPPGGGSFRTPIQEEQADAHSTLAKI

SEQ ID NO: 297 - protein, 188 aa, Artificial Sequence
  Synthetic TNFRSF8 transcript variant 1 NM_001243_4
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 298 - protein, 42 aa, Artificial Sequence; identical to SEQ ID NO: 53
  Synthetic TNFRSF9 NM_001561_5

SEQ ID NO: 299 - protein, 60 aa, Artificial Sequence; identical to SEQ ID NO: 62
  Synthetic TNFRSF14 transcript variant 1 NM_003820_3

SEQ ID NO: 300 - protein, 58 aa, Artificial Sequence
  Synthetic TNFRSF18 transcript variant 1 NM_004195_2
QLGLHIWQLRSQCMWPRETQLLLEVPPSTEDARSCQFPEEERGERSAEEKGRLGDLWV

SEQ ID NO: 301 - protein, 51 aa, Artificial Sequence
  Synthetic TNFRSF18 transcript variant 3_NM_148902_1
QLGLHIWQLRKTQLLLEVPPSTEDARSCQFPEEERGERSAEEKGRLGDLWV

SEQ ID NO: 302 - protein, 23 aa, Artificial Sequence
  Synthetic Linker
GSGGSEGGGSEGGAATAGSGSGS

SEQ ID NO: 303 - protein, 5 aa, Artificial Sequence
  Synthetic TRAF1, TRAF2, and TRAF3 consensus binding sequence
  misc_feature (2)..(2): Xaa can be any naturally occurring amino acid
  misc_feature (4)..(4): Xaa can be any naturally occurring amino acid
PXQXT

SEQ ID NO: 304 - protein, 4 aa, Artificial Sequence
  Synthetic TRAF2 consensus binding sequence
  misc_feature (2)..(3): Xaa can be any naturally occurring amino acid
SXXE

SEQ ID NO: 305 - protein, 6 aa, Artificial Sequence
  Synthetic TRAF6 consensus binding sequence
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
QXPXEX

SEQ ID NO: 306 - protein, 4 aa, Artificial Sequence
  Synthetic Box1 motif
  misc_feature (2)..(3): Xaa can be any naturally occurring amino acid
PXXP

SEQ ID NO: 307 - protein, 4 aa, Artificial Sequence
  Synthetic Shc phosphotyrosine-binding binding motif
  misc_feature (2)..(3): Xaa can be any naturally occurring amino acid
NXXY

SEQ ID NO: 308 - protein, 4 aa, Artificial Sequence
  Synthetic STAT3 consensus binding sequence
  misc_feature (2)..(3): Xaa can be any naturally occurring amino acid
YXXQ

SEQ ID NO: 309 - protein, 4 aa, Artificial Sequence
  Synthetic STAT5 recruitment sequence
YLPL

SEQ ID NO: 310 - protein, 4 aa, Artificial Sequence
  Synthetic STAT5 consensus recruitment sequence
  misc_feature (1)..(1): Xaa is phosphorylated tyrosine
  misc_feature (3)..(3): Xaa can be any naturally occurring amino acid
XLXL

SEQ ID NO: 311 - protein, 570 aa, Influenza virus
  misc_feature (1)..(570): Influenze A HA from H1N1
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRRRKKRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 312 - protein, 470 aa, Influenza virus
  misc_feature (1)..(470): Influenze A NA from H10N7
MNPNQKLFALSGVAIALSILNLLIGISNVGLNVSLHLKGSSDQDKNWTCTSVTQNNTTLIENTYVNNTTVINKGTGTTKQNYLMLNKSLCKVEGWVVVAKDNAIRFGESEQIIVTREPYVSCDPLGCKMYALHQGTTIRNKHSNGTIHDRTAFRGLISTPLGSPPVVSNSDFLCVGWSSTSCHDGIGRMTICVQGNNNNATATVYYDRRLTTTIKTWAGNILRTQESECVCHNGTCVVIMTDGSASSQAHTKVLYFHKGLVIKEEALKGSARHIEECSCYGHNSKVTCVCRDNWQGANRPVIEIDMNAMEHTSQYLCTGVLTDTSRPSDKSMGDCNNPITGSPGAPGVKGFGFLDSDNTWLGRTISPRSRSGFEMLKIPNAGTDPNSRITERQEIVDNNNWSGYSGSFIDYWDESSVCYNPCFYVELIRGRPEEAKYVWWTSNSLVALCGSPISVGSGSFPDGAQIQYFS

SEQ ID NO: 313 - protein, 523 aa, Artificial Sequence
  Synthetic MV(ed)-F-delta-30
MSIMGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 314 - protein, 599 aa, Artificial Sequence
  Synthetic MV(ed)-H-delta-18
MGSRIVINREHLMIDRPYVLLAVLFVMSLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPNLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 315 - protein, 593 aa, Artificial Sequence
  Synthetic MV(ed)-H-delta-24
MNREHLMIDRPYVLLAVLFVMSLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPNLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 316 - DNA, 477 bp, Artificial Sequence
  Synthetic hGH polyA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctgttgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 317 - DNA, 49 bp, Artificial Sequence
  Synthetic SPA1
aataaaagatctttattttcattagatctgtgtgttggttttttgtgtg

SEQ ID NO: 318 - DNA, 120 bp, Artificial Sequence
  Synthetic SPA2
aataaaatatctcagagctctagacatctgtgtgttggttttttgtgtgtagtaatgaggatctggagatattgaagtatcttccggacgactaacagctgtcattggcggatcttaata

SEQ ID NO: 319 - DNA, 295 bp, Artificial Sequence
  Synthetic b-globin polyA spacer B
atctcaagagtggcagcggtcttgagtggcagcggcggtatacggcagcggcatgtaactagctcctcagtggcagcgatgaggaggcaataaaggaaattgattttcattgcaatagtgtgttggaattttttgtgtctctcaaggttctgttaagtaactgaacccaatgtcgttagtgacgcttagctcttaagaggtcactgacctaacaatctcaagagtggcagcggtcttgagtggcagcggcggtatacggcagcgctatctaagtagtaacaagtagcgtggggca

SEQ ID NO: 320 - DNA, 512 bp, Artificial Sequence
  Synthetic b-globin polyA spacer A
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttaataaaggaaattgattttcattgcaatagtgtgttggaattttttgtgtctctcacacgtagtgggccatcgccctgatagacggtttttcgccctttgacgttggagtccacgttcttcgatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttagaattt

SEQ ID NO: 321 - DNA, 243 bp, Artificial Sequence
  Synthetic 250 cHS4 insulator v1
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacgtggggggatacggggaaaagctt

SEQ ID NO: 322 - DNA, 243 bp, Artificial Sequence
  Synthetic 250 cHS4 insulator v2
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcgtgcagacacgtggggggatacggggaaaagctt

SEQ ID NO: 323 - DNA, 650 bp, Artificial Sequence
  Synthetic 650 cHS4 insulator
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctctgcatgtttgatggtgtatggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccagcgtgcccactggccatgtccctcagtgccacatccccacagttcttcatcacctccagggacggtgacccccccacctccgtgggcagctgtgccactgcagcaccgctctttggagaagataaatcttgctaaatccagcccgaccctcccctggcacaacataaggccattatctctcatccaactccaggacggagtcagtgagaatatt

SEQ ID NO: 324 - DNA, 420 bp, Artificial Sequence
  Synthetic 400 cHS4 insulator
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccac

SEQ ID NO: 325 - DNA, 949 bp, Artificial Sequence
  Synthetic 650 cHS4 insulator and b-globin polyA spacer B
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctctgcatgtttgatggtgtatggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccagcgtgcccactggccatgtccctcagtgccacatccccacagttcttcatcacctccagggacggtgacccccccacctccgtgggcagctgtgccactgcagcaccgctctttggagaagataaatcttgctaaatccagcccgaccctcccctggcacaacataaggccattatctctcatccaactccaggacggagtcagtgagaatattgcgatgccccacgctacttgttactacttagatagcgctgccgtataccgccgctgccactcaagaccgctgccactcttgagattgttaggtcagtgacctcttaagagctaagcgtcactaacgacattgggttcagttacttaacagaaccttgagagacacaaaaaattccaacacactattgcaatgaaaatcaatttcctttattgcctcctcatcgctgccactgaggagctagttacatgccgctgccgtataccgccgctgccactcaagaccgctgccactcttgagat

SEQ ID NO: 326 - DNA, 949 bp, Artificial Sequence
  Synthetic b-globin polyA spacer B and 650 cHS4 insulator
atctcaagagtggcagcggtcttgagtggcagcggcggtatacggcagcggcatgtaactagctcctcagtggcagcgatgaggaggcaataaaggaaattgattttcattgcaatagtgtgttggaattttttgtgtctctcaaggttctgttaagtaactgaacccaatgtcgttagtgacgcttagctcttaagaggtcactgacctaacaatctcaagagtggcagcggtcttgagtggcagcggcggtatacggcagcgctatctaagtagtaacaagtagcgtggggcatcgcgagctcacggggacagcccccccccaaagcccccagggatggtcgtacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctctgcatgtttgatggtgtatggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccagcgtgcccactggccatgtccctcagtgccacatccccacagttcttcatcacctccagggacggtgacccccccacctccgtgggcagctgtgccactgcagcaccgctctttggagaagataaatcttgctaaatccagcccgaccctcccctggcacaacataaggccattatctctcatccaactccaggacggagtcagtgagaatatt

SEQ ID NO: 327 - DNA, 15 bp, Artificial Sequence
  Synthetic Kozak sequence
  misc_feature (1)..(3): nnn, if present, is GCC
  misc_feature (10)..(10): n is A or G
nnngccgccnccatg

SEQ ID NO: 328 - DNA, 9 bp, Artificial Sequence
  Synthetic Kozak sequence
  misc_feature (7)..(7): n is T or U
  misc_feature (9)..(9): n, if present, is G
ccaccangn

SEQ ID NO: 329 - DNA, 9 bp, Artificial Sequence
  Synthetic Kozak-type sequence 2
  misc_feature (7)..(7): n is T or U
  misc_feature (9)..(9): n, if present, is G
ccgccangn

SEQ ID NO: 330 - DNA, 13 bp, Artificial Sequence
  Synthetic Kozak-type sequence 3
  misc_feature (11)..(11): n is T or U
  misc_feature (13)..(13): n, if present, is G
gccgccgccangn

SEQ ID NO: 331 - DNA, 13 bp, Artificial Sequence
  Synthetic Kozak sequence
  misc_feature (11)..(11): n is T or U
  misc_feature (13)..(13): n, if present, is G
gccgccaccangn

SEQ ID NO: 332 - RNA, 12 nt, Artificial Sequence
  Synthetic Kozak sequence
gccgccaccaug

SEQ ID NO: 333 - DNA, 28 bp, Mus musculus
  misc_feature (1)..(28): SIBR (synthetic inhibitory BIC-derived RNA)
ctggaggcttgctgaaggctgtatgctg

SEQ ID NO: 334 - DNA, 45 bp, Mus musculus
  misc_feature (1)..(45): 3 microRNA flanking sequence of miR-155
caggacacaaggcctgttactagcactcacatggaacaaatggcc

SEQ ID NO: 335 - DNA, 19 bp, Artificial Sequence
  Synthetic synthetic DNA encoding stem
gttttggccactgactgac

SEQ ID NO: 336 - protein, 511 aa, Artificial Sequence
  Synthetic VSV-G envelope protein
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 337 - protein, 563 aa, Artificial Sequence
  Synthetic Baboon retroviral envelope glycoprotein
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 338 - protein, 654 aa, Artificial Sequence
  Synthetic MuLV envelope protein
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 339 - protein, 545 aa, Artificial Sequence
  Synthetic Baboon retroviral envelope glycoprotein -delta-R (HA)
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHA

SEQ ID NO: 340 - protein, 546 aa, Artificial Sequence
  Synthetic Baboon retroviral envelope glycoprotein -delta-R (HAM)
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 341 - protein, 905 aa, Artificial Sequence
  Synthetic Fusion of anti-CD3 scFV from UCHT1 to MuLV envelope protein
MARSTLSKPPQDKINPWKPLIVMGVLLGVGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSAAAIEGRMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 342 - DNA, 9 bp, Artificial Sequence
  Synthetic Kozak-type sequence
gccgccacc

SEQ ID NO: 343 - DNA, 9 bp, Artificial Sequence
  Synthetic triple stop sequence
taatagtga

SEQ ID NO: 344 - DNA, 191 bp, Artificial Sequence
  Synthetic WPRE
gtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 345 - protein, 654 aa, Artificial Sequence
  Synthetic MuLVSUx
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTIEGREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 346 - protein, 905 aa, Artificial Sequence
  Synthetic UMuLVSUx
MARSTLSKPPQDKINPWKPLIVMGVLLGVGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSAAAIEGRMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTIEGREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 347 - protein, 770 aa, Artificial Sequence
  Synthetic UCHT1-(G4S)3-VSVG
MKCLLYLAFLFIGVNCDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 348 - protein, 767 aa, Artificial Sequence
  Synthetic UCHT1-hinge-VSVG
MKCLLYLAFLFIGVNCDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSEPKSCDKTHTCPKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 349 - protein, 6 aa, Artificial Sequence
  Linker
GSTSGS

SEQ ID NO: 350 - DNA, 1179 bp, Artificial Sequence
  Synthetic EF1a
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 351 - DNA, 511 bp, Artificial Sequence
  Synthetic PGK
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 352 - DNA, 30 bp, Artificial Sequence
  Synthetic 1x NFAT
ggaggaaaaactgtttcatacagaaggcgt

SEQ ID NO: 353 - DNA, 372 bp, Artificial Sequence
  Synthetic 6X NFAT
ataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctcttaaatcactactcatagtaacctcaactcctg

SEQ ID NO: 354 - DNA, 114 bp, Artificial Sequence
  Synthetic minIL2P
cattttgacacccccataatatttttccagaattaacagtataaattgcatctcttgttcaagagttccctatcactctcttaaatcactactcatagtaacctcaactcctga

SEQ ID NO: 355 - DNA, 373 bp, Artificial Sequence
  Synthetic 6X NFAT-minIL2P
ataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctcttaaatcactactcatagtaacctcaactcctga

SEQ ID NO: 356 - DNA, 295 bp, Artificial Sequence; identical to SEQ ID NO: 319
  Synthetic b-globin polyA spacer B

SEQ ID NO: 357 - DNA, 512 bp, Artificial Sequence; identical to SEQ ID NO: 320
  Synthetic b-globin polyA spacer A

SEQ ID NO: 358 - DNA, 243 bp, Artificial Sequence; identical to SEQ ID NO: 321
  Synthetic 250 cHS4 insulator v1

SEQ ID NO: 359 - DNA, 243 bp, Artificial Sequence; identical to SEQ ID NO: 322
  Synthetic 250 cHS4 insulator v2

SEQ ID NO: 360 - DNA, 650 bp, Artificial Sequence; identical to SEQ ID NO: 323
  Synthetic 650 cHS4 insulator

SEQ ID NO: 361 - DNA, 420 bp, Artificial Sequence; identical to SEQ ID NO: 324
  Synthetic 400 cHS4 insulator

SEQ ID NO: 362 - DNA, 949 bp, Artificial Sequence; identical to SEQ ID NO: 325
  Synthetic 650 cHS4 insulator and b-globin polyA spacer B

SEQ ID NO: 363 - DNA, 949 bp, Artificial Sequence; identical to SEQ ID NO: 326
  Synthetic b-globin polyA spacer B and 650 cHS4 insulator

SEQ ID NO: 364 - DNA, 1761 bp, Artificial Sequence
  Synthetic E006-T016-S186-S050 eTag
atggctctgcctgtgacagctctgctgctgcctctggctctgcttctgcatgctgctagacctagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagttgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacatgtaccggcagaggccccgacaactgcattcagtgcgcccactacatcgacggccctcactgcgtgaaaacatgtcctgctggcgtgatgggagagaacaacaccctcgtgtggaagtatgccgacgccggacacgtgtgccacctgtgtcaccctaattgcacctatggctgtaccggccctggcctggaaggctgtccaacaaacggcctggaacggatcgcccggctggaagagaaagtgaaaacactgaaggcccagaacagcgagctggcctccacagccaacatgctgagagaacaggtggcccagctgaagcagaaagtcggcggctctaatctgggcagcgtgtacatctacgtgctgctgatcgtgggcacactcgtgtgcggaatcgtgctgggctttctgtttggcggcagcagatggcagttccccgctcactatcggagactgagacacgccctgtggccatctctgcccgatctgcaccgggtgctgggccagtatctgagagataccgccgctctgtctccacctaaggccaccgtgtccgatacatgcgaggaagtggaacccagcctgctggaaatcctgcccaagagcagcgagagaacccctctgcctctgtgttctagccaggctcagatggactaccgcagactgcagcctagctgcctgggaacaatgcccctgtctgtgtgtcctcccatggccgagagcggcagctgctgcacaacccacattgccaaccacagctacctgcctctgagctactggcagcaacctggcggatcaaagaaggtggccaagaagcccaccaacaaggcccctcatcctaagcaagagccccaagagatcaacttccccgacgatctgcccggcagcaatactgctgctcccgtgcaagaaaccctgcacggttgtcagcccgtgacacaagaggacggcaaagaaagccggatcagcgtccaagaacggcagtaa

SEQ ID NO: 365 - DNA, 239 bp, Artificial Sequence
  Synthetic No Insulator spacer C
atctcaagagtggcagcggtcttgagtggcagcggcggtatacggcagcggcatgtaactagctcctcagtggcagcgatgaggaggcaggttctgttaagtaactgaacccaatgtcgttagtgacgcttagctcttaagaggtcactgacctaacaatctcaagagtggcagcggtcttgagtggcagcggcggtatacggcagcgctatctaagtagtaacaagtagcgtggggca

SEQ ID NO: 366 - protein, 905 aa, Artificial Sequence; identical to SEQ ID NO: 346
  Synthetic UMuLVSUx

SEQ ID NO: 367 - protein, 770 aa, Artificial Sequence; identical to SEQ ID NO: 347
  Synthetic UCHT1-(G4S)3-VSVG

SEQ ID NO: 368 - protein, 376 aa, herpes simplex virus 7
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFVLDYDQSPAGCRDALLQLTSGMVQTHVTTPGSIPTICDLARTFAREMGEAN

SEQ ID NO: 369 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 370 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 371 - protein, 12 aa, Homo sapiens
GQNDTSQTSSPS

SEQ ID NO: 372 - protein, 654 aa, Artificial Sequence; identical to SEQ ID NO: 345
  Synthetic MuLVSUx

SEQ ID NO: 373 - protein, 20 aa, Artificial Sequence
  Synthetic
GGGGSGGGGSGGGGSGGGGS

SEQ ID NO: 374 - protein, 546 aa, Nipah henipavirus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 375 - protein, 602 aa, Nipah henipavirus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

(19) United States
(12) Patent Application Publication
(10) Pub. No.: US 2023/0111159 A1
(43) Pub. Date: Apr. 13, 2023

(54) METHODS AND COMPOSITIONS FOR THE DELIVERY OF MODIFIED LYMPHOCYTE AGGREGATES

(71) Applicant: Exuma Biotech Corp., West Palm Beach, FL (US)

(72) Inventors: Gregory Ian Frost, West Palm Beach, FL (US); Frederic Vigant, Lake Worth, FL (US); Anirban Kundu, George Town, Grand Cayman (KY); John R. Henkelman, III, West Palm Beach, FL (US); Sidharth Kerkar, Boynton Beach, FL (US); Gregory Schreiber, Palm Beach Gardens, FL (US)

(73) Assignee: Exuma Biotech Corp, West Palm Beach, FL (US)

(21) Appl. No.: 17/905,649

(22) PCT Filed: Mar. 4, 2021
(86) PCT No.: PCT/US2021/020922

§ 371 Date: Sep. 5, 2022

Related U.S. Application Data
(63) Continuation-in-part of application No. PCT/US2020/048843, filed on Aug. 31, 2020.
(60) Provisional application No. 62/985,741, filed on Mar. 5, 2020.
(60) Provisional application No. 63/136,177, filed on Jan. 11, 2021.
(60) Provisional application No. 63/200,329, filed on Mar. 1, 2021.

(51) Int. Cl. A61K 35/17; C12N 15/86; A61K 35/15; A61K 45/06; C07K 14/725; A61P 35/00

(52) U.S. Cl. CPC A61K 35/17; C12N 15/86; A61K 35/15; A61K 45/06; C07K 14/7051; A61P 35/00; C12N 2740/15043

(57) ABSTRACT

The present disclosure provides methods and compositions for genetically modifying lymphocytes, for example T cells and/or NK cells. In some embodiments, the methods include reaction mixtures, and resulting cell formulations, that are created using whole blood, or a component thereof that is not a PBMC, and additionally comprise T cells and recombinant retroviral particles having polynucleotides that encode a CAR. In some embodiments, modified lymphocytes are reintroduced into a subject subcutaneously. In some embodiments, polynucleotides that provide T cells the ability to regulate cell survival and proliferation in response to binding to a CAR, are provided.

### CROSS-REFERENCE TO RELATED APPLICATIONS

This application claims priority to U.S. Provisional Application No. 62/985,741, filed Mar. 5, 2020; International Application No. PCT/US2020/048843, filed Aug. 31, 2020; U.S. Provisional Application No. 63/136,177, filed Jan. 11, 2021; and U.S. Provisional Application No. 63/200,329, filed Mar. 1, 2021; International Application No. PCT/US2020/048843, filed Aug. 31, 2020 is a continuation-in-part of International Application No. PCT/US2019/049259, filed Sep. 2, 2019; and claims the benefit of U.S. Provisional Application No. 62/894,849, filed Sep. 1, 2019; U.S. Provisional Application No. 62/894,852, filed Sep. 1, 2019; U.S. Provisional Application No. 62/894,853, filed Sep. 1, 2019; U.S. Provisional Application No. 62/894,926, filed Sep. 2, 2019; U.S. Provisional Application No. 62/943,207, filed Dec. 3, 2019; U.S. Provisional Application No. 62/985,741, filed Mar. 5, 2020; International Application No. PCT/US2019/049259 is a continuation-in-part of International Application No. PCT/US2018/051392 filed Sep. 17, 2018; and claims the benefit of U.S. Provisional Application No. 62/726,293, filed Sep. 2, 2018; U.S. Provisional Application No. 62/726,294, filed Sep. 2, 2018; U.S. Provisional Application No. 62/728,056 filed Sep. 6, 2018; U.S. Provisional Application No. 62/732,528, filed Sep. 17, 2018; U.S. Provisional Application No. 62/821,434, filed Mar. 20, 2019; and U.S. Provisional Application No. 62/894,853, filed Sep. 1, 2019; and International Application No. PCT/US2018/051392 is a continuation-in-part of International Application No. PCT/US2018/020818, filed Mar. 3, 2018; and claims the benefit of U.S. Provisional Application No. 62/560,176, filed Sep. 18, 2017; U.S. Provisional Application No. 62/564,253, filed Sep. 27, 2017; U.S. Provisional Application No. 62/564,991, filed Sep. 28, 2017; and U.S. Provisional Application No. 62/728,056, filed Sep. 6, 2018; International Application No. PCT/US2018/020818 is a continuation-in-part of International Application No. PCT/US2017/023112 filed Mar. 19, 2017; a continuation-in-part of International Application No. PCT/US2017/041277 filed Jul. 8, 2017; a continuation-in-part of U.S. Application No. 15/462,855 filed Mar. 19, 2017; and a continuation-in-part of U.S. Application No. 15/644,778 filed Jul. 8, 2017; and claims the benefit of U.S. Provisional Application No. 62/467,039 filed Mar. 3, 2017; U.S. Provisional Application No. 62/560,176 filed Sep. 18, 2017; U.S. Provisional Application No. 62/564,253 filed Sep. 27, 2017; and U.S. Provisional Application No. 62/564,991 filed Sep. 28, 2017; International Application No. PCT/US2017/023112 claims the benefit of U.S. Provisional Application No. 62/390,093, filed Mar. 19, 2016; U.S. Provisional Application No. 62/360,041, filed Jul. 8, 2016; and U.S. Provisional Application No. 62/467,039, filed Mar. 3, 2017; International Application No. PCT/US2017/041277 claims the benefit of International Application No. PCT/US2017/023112, filed Mar. 19, 2017; U.S. Patent Application No. 15/462,855, filed Mar. 19, 2017; U.S. Provisional Application No. 62/360,041, filed Jul. 8, 2016; and U.S. Provisional Application No. 62/467,039, filed Mar. 3, 2017; U.S. application Ser. No. 15/462,855 claims the benefit of U.S. Provisional Application No. 62/390,093, filed Mar. 19, 2016; U.S. Provisional Application No. 62/360,041, filed Jul. 8, 2016; and U.S. Provisional Application No. 62/467,039, filed Mar. 3, 2017; and U.S. application Ser. No. 15/644,778 is a continuation-in-part of International Application No. PCT/US2017/023112, filed Mar. 19, 2017; and a continuation-in-part of U.S. Patent application Ser. No. 15/462,855, filed Mar. 19, 2017; and claims the benefit of U.S. Provisional Application No. 62/360,041, filed Jul. 8, 2016, and U.S. Provisional Application No. 62/467,039, filed Mar. 3, 2017. These applications are incorporated by reference herein in their entireties.

### SEQUENCE LISTING

This application hereby incorporates by reference the material of the electronic Sequencing Listing filed concurrently herewith. The materials in the electronic Sequence Listing is submitted as a text (.txt) file entitled “F1_003_WO_02_Sequence_Listing” created on Mar. 3, 2021, which has a file size of 454 KB, and is herein incorporated by reference in its entirety.

### FIELD OF INVENTION

This disclosure relates to the field of immunology, or more specifically, to the genetic modification of T lymphocytes or other immune cells, and methods of controlling proliferation of such cells.

### BACKGROUND OF THE DISLOSURE

Lymphocytes isolated from a subject (e.g., patient) can be activated in vitro and genetically modified to express synthetic proteins that enable redirected engagement with other cells and environments based upon the genetic programs incorporated. Examples of such synthetic proteins include engineered T cell receptors (TCRs) and chimeric antigen receptors (CARs). One CAR that is currently used is a fusion of an extracellular recognition domain (e.g., an antigen-binding domain), a transmembrane domain, and one or more intracellular signaling domains encoded by a replication incompetent recombinant retrovirus.

While recombinant retroviruses have shown efficacy in infecting non-dividing cells, resting CD4 and CD8 lymphocytes are refractory to genetic transduction by these vectors. To overcome this difficulty, these cells are typically activated in vitro using stimulation reagents before genetic modification with the CAR gene vector can occur. Following stimulation and transduction, the genetically modified cells are expanded in vitro and subsequently reintroduced into a lymphodepleted patient. Upon antigen engagement in vivo, the intracellular signaling portion of the CAR can initiate an activation-related response in an immune cell and release of cytolytic molecules to induce target cell death.

Such current methods require extensive manipulation and manufacturing of proliferating T cells outside the body prior to their reinfusion into the patient, as well as lymphodepleting chemotherapy to free cytokines and deplete competing receptors to facilitate T cell engraftment. Such CAR therapies further cannot be controlled for propagation rate in vivo once introduced into the body, nor safely directed towards targets that are also expressed outside the tumor. As a result, CAR therapies today are typically infused from cells expanded ex vivo from 12 to 28 days using doses from 1×10⁵ to 1×10⁸ cells/kg and are directed towards targets, for example tumor targets, for which off tumor on target toxicity is generally acceptable. These relatively long ex vivo expansion times create issues of cell viability and sterility, as well as sample identity in addition to challenges of scalability. Thus, there are significant needs for a safer, more effective scalable T cell or NK cell therapy. Further reduction in the complexity and time required for such methods would be highly desirable, especially if such methods allow a subject to have their blood collected, for example within an infusion center, and then reintroduced into the subject that same day. Furthermore, simpler and quicker methods alone or methods that require fewer specialized instruments, could democratize these cell therapy processes, which are currently performed regularly only at highly specialized medical centers.

Since our understanding of processes that drive transduction, proliferation and survival of lymphocytes is central to various potential commercial uses that involve immunological processes, there is a need for improved methods and compositions for studying lymphocytes. For example, it would be helpful to identify methods and compositions that can be used to better characterize and understand how lymphocytes can be genetically modified and the factors that influence their survival and proliferation. Furthermore, it would be helpful to identify compositions that drive lymphocyte proliferation and survival. Such compositions could be used to study the regulation of such processes. In addition to methods and compositions for studying lymphocytes, there is a need for improved viral packaging cell lines and methods of making and using the same. For example, such cell lines and methods would be useful in analyzing different components of recombinant viruses, such as recombinant retroviral particles, and for methods that use packaging cells lines for the production of recombinant retroviral particles.

Additionally, there remains a need for improved compositions and methods for inducing proliferation and/or survival of lymphocytes in the blood, organs, and tissue, and preferentially and specifically, in the tumor microenvironment. Previous methods have used cells with constitutively expressing CARs that, upon binding target antigen, induce expression of secreted cytokines under the control of a CAR-stimulated inducible promoter. These secreted cytokines bind to and stimulate T cells and NK cells nonspecifically, thus reducing the amount of cytokines available to stimulate the CAR T cells or NK cells. The cytokines also can diffuse away further reducing the cytokines available to stimulate the CAR T cells or NK cells. These prior methods usually required multiple transductions of transcriptional units on separate vectors, and required long blood cell-processing times, therefore requiring cancer patients to wait for days, weeks, and even months after their blood is collected, to receive their genetically engineered blood cells. Prior methods that have performed CAR-T cell transduction in one step that used a vector encoding more than one transcriptional unit, generated low viral titer and/or resulted in low expression of one or more of the transcriptional units, each of which are key impediments to commercialization as a general treatment method. Accordingly, there remains a need for more efficient methods to generate CAR-T cells that survive and proliferate in the blood, organs, and tissue, and preferentially and specifically, in the inhibitory tumor microenvironment.

### SUMMARY

Provided herein are methods, uses, compositions, and kits that simplify and speed up the process of genetically modifying lymphocytes, in illustrative embodiments T cells and/or NK cells. Some aspects and embodiments provided herein, are well-suited for point-of-care cell processing and do not require transport of cells to specialized processing facilities. Furthermore, methods, uses, compositions, and kits provided herein help overcome issues related to the effectiveness and safety of methods for transducing and/or modifying and in illustrative embodiments genetically modifying lymphocytes such as T cells and/or NK cells. Certain embodiments of such methods are useful for performing adoptive cell therapy with these cells. Accordingly, in some aspects, provided herein are methods, compositions, and kits for modifying lymphocytes, especially T cell and/or NK cells, and/or for regulating the activity of transduced, genetically modified, and/or modified T cells and/or NK cells. Such methods, compositions, and kits provide improved efficacy and safety over current technologies, especially with respect to T cells and/or NK cells that express engineered T cell receptors (TCRs), chimeric antigen receptors (CARs), and in illustrative embodiments microenvironment restricted biologic (“MRB”) CARs. Transduced and/or modified and in illustrative embodiments genetically modified T cells and/or NK cells that are produced by and/or used in methods provided herein, include functionality and combinations of functionality, in illustrative embodiments delivered from retroviral (e.g. lentiviral) genomes via retroviral (e.g. lentiviral) particles, that provide improved features for such cells and for methods that utilize such cells, such as research methods, commercial production methods, and adoptive cellular therapy. For example, such cells can be produced in less time ex vivo, and that have improved growth properties that can be better regulated. In illustrative embodiments, such methods, uses, compositions, and kits include, or are adapted for intramuscular or in further illustrative embodiments, subcutaneous delivery to a subject.

In some aspects, methods are provided for transducing and/or modifying and in illustrative embodiments genetically modifying lymphocytes such as T cells and/or NK cells, and in illustrative embodiments, ex vivo methods for transducing, genetically modifying, and/or modified resting T cells and/or NK cells. Some of these aspects can be performed much more quickly than previous methods, which can facilitate more efficient research, more effective commercial production, and improved methods of patient care. Methods, uses, compositions, and kits provided herein, can be used as research tools, in commercial production, and in adoptive cellular therapy with transduced and/or modified and in illustrative embodiments genetically modified T cells and/or NK cells expressing a TCR or a CAR.

With respect to methods, uses and compositions provided herein that relate to transduction of lymphocytes such as T cells and/or NK cells, methods, and associated uses and compositions, are provide herein that include transduction reactions of enriched PBMCs, TNCs, or transduction reactions without prior cellular enrichment, such as in whole blood that are simplified and quicker methods for performing ex-vivo cell processing, for example for CAR-T therapy. Such methods require less specialized instrumentation and training Furthermore, such methods reduce the risk of non-targeted cell transduction compared to in vivo transduction methods. Furthermore, provided herein are methods, uses, and compositions, including embodiments of the methods immediately above, that include certain target inhibitory RNAs, activation elements, polypeptide lymphoproliferative elements, pseudotyping elements, and artificial antigen presenting cells that can be optionally combined with any other aspects provided herein to provide powerful methods, uses, and compositions for driving expansion of lymphocytes, especially T cells and/or NK cells in vitro, ex vivo, and in vivo. In some embodiments, the modified lymphocytes are capable of engrafting in a lymphoreplete environment. In some embodiments, patients or subjects are not lymphodepleted prior to reinfusion with modified and/or genetically modified T cells and or NK cells

In some aspects and embodiments, provided herein are genetic constructs that are especially well-suited to provide genetically modified T cells and/or NK cells the ability to survive and proliferate in a more controllable manner In contrast to constitutive promoters operably linked to lymphoproliferative elements or inducible promoters operably linked to secreted cytokines, such aspects and embodiments provide inducible promoters operably linked to membrane-bound lymphoproliferative elements, that when induced by CAR-binding to its target, can induce proliferation of T cells and/or NK cells, such as, for example, those present in the tumor microenvironment.

Further details regarding aspects and embodiments of the present disclosure are provided throughout this patent application. Sections and section headers are for ease of reading and are not intended to limit combinations of disclosure, such as methods, compositions, and kits or functional elements therein across sections.

### BRIEF DESCRIPTION OF THE DRAWINGS

FIGS. 1A-1G are flowcharts of non-limiting exemplary cell processing workflows. FIG. 1A is a flow chart of a process that uses a system with PBMC isolation before the contacting of T cells and NK cells in the PBMCs with retroviral particles. An optional step to deplete unwanted cells can be initiated prior to PBMC isolation. FIG. 1B is a flow chart of a process that performs total nucleated cell (TNC) isolation before the contacting of T cells and NK cells in the total nucleated cells, with retroviral particles. An optional step to deplete unwanted cells can be initiated after the TNC isolation and prior to the optional PBMC isolation. FIG. 1C is a flow chart of a process in which no blood cell fractionation or enrichment is performed before T cells and NK cells in the whole blood are contacted with retroviral particles, and a PBMC isolation is performed after the contacting and optional incubation. An optional step to deplete unwanted cells can be initiated prior to PBMC isolation. FIG. 1D is a flow chart of a process in which no blood cell fractionation or enrichment is performed before T cells and NK cells in the whole blood are contacted with retroviral particles, and a TNC isolation/concentration is performed after the contacting and optional incubation, in illustrative embodiments using filtration, for example using a leukoreduction filter assembly. An optional step to deplete unwanted cells followed by a filtration process can be performed prior to the TNC isolation/concentration step. FIG. 1E is a flow chart of a process that performs TNC isolation before the “Cold Contacting” of T cells and NK cells in the total nucleated cells, with retroviral particles. An optional step to deplete unwanted cells can be initiated prior to TNC isolation. Another optional step is a secondary incubation which is optionally combined with a coarse filtration to capture lymphocyte aggregates and/or to remove unwanted cells. FIG. 1F is a flow chart of a process that performs TNC isolation before the “Cold Contacting” of T cells and NK cells in the total nucleated cells, with retroviral particles. An optional step to deplete unwanted cells can be initiated prior to TNC isolation. Another optional step is a secondary incubation. FIG. 1G is a flow chart of a process in which no blood cell fractionation or enrichment is performed before T cells and NK cells in the whole blood are contacted with retroviral particles, and a coarse filter is used to capture aggregates that will comprise T and/or NK cells. Any one or more of the wash steps are optional. Each of these cell processing workflows could be used for rPOC cell therapy.

FIG. 2 is a diagram of a non-limiting exemplary leukoreduction filter assembly (200) with associated blood processing bags, tubes, valves, and filter enclosure (210) comprising a leukoreduction filter set.

FIG. 3 is a diagram of a non-limiting exemplary transduction assembly (301) with associated tubing, syringes, and incubation bag (314).

FIG. 4 is a diagram of a non-limiting exemplary leukoreduction filter assembly (400) with associated blood processing bags, tubes, valves, and filter enclosure (410) comprising a leukoreduction filter set.

FIG. 5 shows a contour FACS plot of the expression of CD3 and eTag on the live lymphocyte population at Day 7 post-transduction of whole blood for 4 hours with F1-3-23GU followed by an isolation of total nucleated cells by TNC filtration using an illustrative leukoreduction filter assembly.

FIG. 6 shows the number of CD3+eTAG+CAR-T cells per 60 μl of peripheral blood in individual mice 7, 14, and 21 days post intravenous CAR-T dosing. Dosed cells were either untransduced or transduced with F1-3-247GU at the indicated MOI.

FIG. 7 shows the number of CD3+eTAG+CAR-T cells per 60 μl of peripheral blood in individual mice 8, 14, and 21 days post subcutaneous CAR-T dosing. Dosed cells were either untransduced or transduced with F1-3-247GU at the indicated MOI.

FIG. 8 shows a graph of the mean tumor volume of Raji tumors in B-NDG mice dosed intravenously on Day 0 with PBMCs that were not transduced (UNT) or that were transduced (TRNSD) by a 4 hour exposure to F1-3-247GU at the indicated MOI. Mice in each group were dosed with either 1 million or 5 million PBMCs as indicated.

FIG. 9 shows a graph of the mean tumor volume of Raji tumors in B-NDG mice dosed subcutaneously on Day 0 with PBMCs that were not transduced (UNT) or that were transduced (TRNSD) by a 4 hour exposure to F1-3-247GU at the indicated MOI. Mice in each group were dosed with either 1 million or 5 million PBMCs as indicated.

FIG. 10 shows a schematic of an illustrative bicistronic lentiviral genomic vector with divergent transcriptional units. A first transcriptional unit comprising an eTagged lymphoproliferative element (eTag:LE) followed by a polyadenylation sequence (PolyA) under the transcriptional control of an NFAT-responsive minimal IL-2 promoter (6× NFAT) is encoded in the reverse orientation. Optionally, an insulator element (Ins) separates the first and second transcriptional units. The second transcriptional unit encodes a CAR (CAR) under the transcriptional control of a constitutive promoter (Promoter) and is encoded in the forward orientation. Triangles shown in dashed lines represent 3 possible locations into any one or more of which, one or more miRNAs could optionally be inserted into the vector. The triangle shown in a dotted line represents 1 possible location in an exon within a promoter such as for EF1-a into which one or more miRNAs could optionally be inserted into the vector. “SA” and “SD” correspond to splice donor and splice acceptor sites.

FIG. 11 shows a graph of the percentage of CD3+CAR+ PMBCs expressing eTag. PBMCs were transduced with the indicated bicistronic lentiviral genomic construct and were fed with CD19-expressing Raji cells every other day beginning on day 7, or left unfed in the absence of exogenous cytokines. CD3+CAR+ cells were assayed by flow cytometry for the expression of eTag each day as indicated.

FIGS. 12A-D show graphs of the fold expansion of CD3+CAR+ PMBCs. PBMCs were transduced with the lentiviral genomic construct F1-3-635 (FIG. 12A), F1-3-637 (FIG. 12B), F1-3-23 (FIG. 12C), or F1-3-247 (FIG. 12D), and either fed with CD19-expressing Raji cells every other day beginning on day 7, or left unfed in the absence of exogenous cytokines. CD3+CAR+ cells were detected by flow cytometry.

FIG. 13 shows a graph of the fold expansion of CD3+CAR+ PMBCs. PBMCs were transduced with the lentiviral genomic constructs F1-3-635, F1-3-637, F1-3-23, or F1-3-635, and were left unfed and culture in the absence of cytokines after day 7.

FIG. 14 shows a graph of the percent viability of CD3+CAR+ PMBCs. PBMCs were transduced with the lentiviral genomic constructs F1-3-635, F1-3-637, F1-3-23, or F1-3-635, and were left unfed and culture in the absence of cytokines after day 7.

FIG. 15 shows a graph of the total flux [p/s] of Raji-luciferase disseminated tumor burden in NSG-(K^{b}D^{b})^{null} (IA)^{null} mice dosed subcutaneously on Day 0 with PBMCs that were not transduced (G1) or that were transduced by exposure of whole blood to F1-3-637GU (G2) or F1-4-713GU (G3) lentiviral particles for 4 hours followed by a PBMC enrichment procedure. Mice in G4 were treated with a half dose of PBMCs from G2 and G3. The genomic vectors of F1-3-637GU and F1-4-713GU encode self-driving CARs to CD19 and CD22, respectively.

FIG. 16 shows a graph of the probability of survival for 8 weeks of the mice in FIG. 23.

FIG. 17 shows total cell recoveries and cell surface marker expression of TNCs transduced with F1-3-637GU after 6 days of culture in CTS media supplemented with rhIL-2. The contacting step of the rPOC cell process was performed as shown in either FIG. 1D (Whole Blood) or FIG. 1B. (On Filter).

FIG. 18 shows a graph of IFN gamma production (pg/ml) by the cells from FIG. 25 as measured by ELISA, after the cells were left untreated (NA), or treated with CHO-S, Raji, or PMA+Ionomycin for 16 hours.

FIG. 19 shows a graph of the total flux [p/s] of Raji-luciferase disseminated tumor burden in NSG mice dosed subcutaneously on Day 0 with PBS (G1), TNCs (G2), PBMCs (G3), or cells that were transduced by exposure of whole blood to F1-3-637GU lentiviral particles for 4 hours followed by a TNC enrichment procedure as shown in FIG. 1D (G4) or a PBMC enrichment procedure as shown in FIG. 1C (G5).

FIG. 20 shows representative FACS contour plots showing CD3 dimmed cells following contacting with increasing concentrations of F1-3-247GU RIPs displaying a CD3 T cell activation element on their surface. Whole blood was contacted with virus for 4 hours before RBCs were lysed and cells were prepared for FACs. FIG. 20A shows FSC-H vs SSC-H, FIG. 20B shows CD3 vs CD4, FIG. 20C shows CD3 vs. CD8.

FIG. 21 is a table showing the percentages of cells with the surface phenotypes as shown from the experiment same experiment described for FIG. 20

FIG. 22 is a table showing the percentages of cells with the surface phenotypes as shown following contacting whole blood with F1-3-247GU RIPs displaying a CD3 T cell activation element on their surface followed by PBMC or TNC isolation.

FIG. 23 shows the biodistribution of TNCs after the cells were isolated from whole blood that had been contacted with F1-3-748GU for 4 hours, and injected into mice. FIG. 23A shows the biodistribution of these cells after they were injected subcutaneously. FIG. 23B shows the biodistribution of these cells after they were injected intravenously.

FIG. 24A shows a dot blot of human CD45 and murine CD45 expression in the blood of an NSG-MHC1/2-DKO mouse 27 days after it was reconstituted with human PBMCs intravenously.

FIG. 24B shows a graph of the number of CD4+CAR+ and CD8+CAR+ cells per ml of blood in mice 27 days after they were injected intravenously and/or subcutaneously with the test articles as indicated. The graph represents the averages from 5 mice in each group.

FIG. 25 shows a graph of the number of CD19+ target cells/ml of blood in mice 21 days after they were injected intravenously and/or subcutaneously with the test articles as indicated. The graph represents the averages from 5 mice in each group.

FIG. 26 shows photomicrographs of H&E-stained skin and subcutaneous tissue from a mice after subcutaneous injection with PBMCs modified by an rPOC cell processing method with F1-3-247GU and injected subcutaneously. Representative fields are shown for Day 1 (FIG. 26A), Day 7 (FIG. 26B), Day 14 (FIG. 26C) and Day 21 (FIG. 26D).

FIG. 27 shows a graph of the mean tumor volume of N87 tumors in B-NDG mice dosed subcutaneously on Day 0 with either 1 million or 5 million TNCs transduced by a 4 hour exposure to F1-6-744GU.

### DEFINITIONS

As used herein, the term “chimeric antigen receptor” or “CAR” or “CARs” refers to engineered receptors, which graft an antigen specificity onto cells, for example T cells, NK cells, macrophages, and stem cells. The CARs of the invention include at least one antigen-specific targeting region (ASTR), a transmembrane domain (TM), and an intracellular activating domain (IAD) and can include a stalk, and one or more co-stimulatory domains (CSDs). In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

As used herein, the term cell “aggregate” means a cluster of cells that adhere to each other.

As used herein, the term “constitutive T cell or NK cell promoter” refers to a promoter which, when operably linked with a polynucleotide that encodes or specifies a gene product, causes the gene product to be produced in a cell under most or all physiological conditions of the cell.

As used herein, the terms “inducible promoter” or “activatable promoter” refer to promoters that when operably linked with a polynucleotide that encodes or specifies a gene product, cause the gene product to be produced in a cell substantially only when a promoter-specific inducer is present in the cell. Inducible promoters have no, or a low level, of basal transcription activity but the transcription activity increases, sometimes greatly, in the presence of an inducing signal.

As used herein, the term “insulator” refers to a cis-regulatory element that mediates intra- and inter-chromosomal interactions and can block interactions between enhancers and promoters. Typically, insulators are between 200 and 2000 base pairs in length and contain clustered binding sites for sequence specific DNA-binding proteins.

As used herein, the term “microenvironment” means any portion or region of a tissue or body that has constant or temporal, physical, or chemical differences from other regions of the tissue or regions of the body. For example, a “tumor microenvironment” as used herein refers to the environment in which a tumor exists, which is the non-cellular area within the tumor and the area directly outside the tumorous tissue but does not pertain to the intracellular compartment of the cancer cell itself. The tumor microenvironment can refer to any and all conditions of the tumor milieu including conditions that create a structural and or functional environment for the malignant process to survive and/or expand and/or spread. For example, the tumor microenvironment can include alterations in conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions a skilled artisan will understand.

As used interchangeably herein, the terms “polynucleotide” and “nucleic acid” refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “antibody” includes polyclonal and monoclonal antibodies, including intact antibodies and fragments of antibodies which retain specific binding to antigen. The antibody fragments can be, but are not limited to, fragment antigen binding (Fab) fragments, Fab′ fragments, F(ab′)₂ fragments, Fv fragments, Fab′-SH fragments, (Fab′)₂ Fv fragments, Fd fragments, recombinant IgG (rIgG) fragments, single-chain antibody fragments, including single-chain variable fragments (scFv), divalent scFv's, trivalent scFv's, and single domain antibody fragments (e.g., sdAb, sdFv, nanobody). The term includes genetically engineered and/or otherwise modified forms of immunoglobulins, such as intrabodies, peptibodies, chimeric antibodies, single-chain antibodies, fully human antibodies, humanized antibodies, fusion proteins including an antigen-specific targeting region of an antibody and a non-antibody protein, heteroconjugate antibodies, multispecific, e.g., bispecific, antibodies, diabodies, triabodies, and tetrabodies, tandem di-scFv's, and tandem tri-scFv's. Unless otherwise stated, the term “antibody” should be understood to include functional antibody fragments thereof. The term also includes intact or full-length antibodies, including antibodies of any class or sub-class, including IgG and sub-classes thereof, IgM, IgE, IgA, and IgD.

As used herein, the term “antibody fragment” includes a portion of an intact antibody, for example, the antigen binding or variable region of an intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily. Pepsin treatment yields an F(ab′)₂ fragment that has two antigen combining sites and is still capable of cross-linking antigen.

As used interchangeably herein, the terms “single-chain Fv,” “scFv,” or “sFv” antibody fragments include the V_{H} and V_{L} domains of antibody, wherein these domains are present in a single polypeptide chain. In some embodiments, the Fv polypeptide further includes a polypeptide linker or spacer between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, “naturally occurring” VH and VL domains refer to VH and VL domains that have been isolated from a host without further molecular evolution to change their affinities when generated in an scFv format under specific conditions such as those disclosed in U.S. Pat. No. 8,709,755 B2 and application WO/2016/033331A1.

As used herein, “antibody mimetic” refers to an organic compound that specifically binds a target sequence and has a structure distinct from a naturally-occurring antibody. Antibody mimetics may comprise a protein, a nucleic acid, or a small molecule, and a skilled artisan can understand when each type is relevant. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g., resistance to enzymatic degradation), and lower production costs. Antibody mimetics include, but are not limited to, an affibody, an afflilin, an affimer, an affitin, an alphabody, an alphamab, an anticalin, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

As used herein, the term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

As used herein, reference to a “cell surface expression system” or “cell surface display system” refers to the display or expression of a protein or portion thereof on the surface of a cell. Typically, a cell is generated that expresses proteins of interest fused to a cell-surface protein. For example, a protein is expressed as a fusion protein with a transmembrane domain

As used herein, the term “element” includes polypeptides, including fusions of polypeptides, regions of polypeptides, and functional mutants or fragments thereof and polynucleotides, including microRNAs and shRNAs, and functional mutants or fragments thereof.

As used herein, the term “region” is any segment of a polypeptide or polynucleotide.

As used herein, a “domain” is a region of a polypeptide or polynucleotide with a functional and/or structural property.

As used herein, the terms “stalk” or “stalk domain” refer to a flexible polypeptide connector region providing structural flexibility and spacing to flanking polypeptide regions and can consist of natural or synthetic polypeptides. A stalk can be derived from a hinge or hinge region of an immunoglobulin (e.g., IgG1) that is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S-S) bonds in the same positions. The stalk may be of natural occurrence or non-natural occurrence, including but not limited to an altered hinge region, as disclosed in U.S. Pat. No. 5,677,425. The stalk can include a complete hinge region derived from an antibody of any class or subclass. The stalk can also include regions derived from CD8, CD28, or other receptors that provide a similar function in providing flexibility and spacing to flanking regions.

As used herein, the term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

As used herein, a “polypeptide” is a single chain of amino acid residues linked by peptide bonds. A polypeptide does not fold into a fixed structure nor does it have any posttranslational modification. A “protein” is a polypeptide that folds into a fixed structure. “Polypeptides” and “proteins” are used interchangeably herein.

As used herein, a polypeptide may be “purified” to remove contaminant components of a polypeptide's natural environment, e.g., materials that would interfere with diagnostic or therapeutic uses for the polypeptide such as, for example, enzymes, hormones, and other proteinaceous or nonproteinaceous solutes. A polypeptide can be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

As used herein, “T cell” includes all types of immune cells expressing CD3 including T-helper cells (CD4⁺ cells), cytotoxic T cells (CD8⁺ cells), T-regulatory cells (Treg) and gamma-delta T cells. NKT cells, which are CD3+, CD56+, and either CD4+ or CD8+, are considered a type of T cells herein. Surface expression of CD3 can be transiently decreased or eliminated in T cells, as has been observed with some of the methods for modifying T cells disclosed herein. Such modified CD4+ or CD8+ lymphocytes that have transiently decreased/absent CD3 surface expression, are still considered T cells in this disclosure. Reference to a “CD” or cluster of differentiation marker, such as CD3+, CD4+, CD8+, CD56+ herein, relates to surface expression of such polypeptide. It will be understood that surface expression is a continuum between positive and negative, and can be assessed by FACS analysis, where cells are determined to be positive or negative based on user cutoffs known in the art. A low and intermediate expression of a surface marker determined by FACS analysis, such as CD3lo or CD3int, are considered surface marker negative (e.g. CD3−) herein.

As used herein, “NK cell” includes lymphocytes that express CD56 on their surface (CD56+ lymphocytes). NKT cells, which are CD3+, CD56+, and either CD4+ or CD8+, are considered a type of NK cells herein.

As used herein, a “cytotoxic cell” includes CD8⁺ T cells, natural-killer (NK) cells, NK-T cells, γδ T cells, a subpopulation of CD4⁺ cells, and neutrophils, which are cells capable of mediating cytotoxicity responses.

As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, etc.).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e g , in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used interchangeably herein, the terms “individual”, “subject”, “host”, and “patient” refer to a mammal, including, but not limited to, humans, murines (e.g., rats, mice), lagomorphs (e.g., rabbits), non-human primates, humans, canines, felines, ungulates (e.g., equines, bovines, ovines, porcines, caprines), etc.

As used herein, the terms “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

As used herein, the term “evolution” or “evolving” refers to using one or more methods of mutagenesis to generate a different polynucleotide encoding a different polypeptide, which is itself an improved biological molecule and/or contributes to the generation of another improved biological molecule. “Physiological” or “normal” or “normal physiological” conditions are conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions, that would be considered within a normal range at the site of administration, or at the tissue or organ at the site of action, to a subject.

As used herein, a “transduced cell” or a “stably transfected cell” is a cell that contains an exogenous nucleic acid(s) that is integrated into the genome of the cell. As used herein, a “genetically modified cell” is a cell that contains an exogenous nucleic acid(s) regardless of whether the exogenous nucleic acid(s) is integrated into the genome of the cell, and regardless of the method used to introduce the exogenous nucleic acid(s) into the cell. Exogenous nucleic acid(s) inside a cell that are not integrated into the genome of the cell can be referred to as “extrachromosomal” herein. As used herein, a “modified cell” is a cell that is associated with a recombinant nucleic acid vector (also called a “gene vector” herein), which in illustrative embodiments is a replication incompetent recombinant retroviral particle (also called a “RIR retroviral particle” or a “RIP” herein), that contains an exogenous nucleic acid, or a cell that has been genetically modified by an exogenous nucleic acid. Typically, in compositions and methods that include a replication incompetent recombinant retroviral particle, a modified cell associates with a replication incompetent recombinant retroviral particle through interactions between proteins on the surface of the cell and proteins on the surface of the replication incompetent recombinant retroviral particle, including pseudotyping elements and/or T cell activation elements. In compositions and methods that include transfection of nucleic acid inside a lipid-based reagent, such as a liposomal reagent, the lipid-based reagent containing nucleic acid, which is a type of recombinant nucleic acid vector, associates with the lipid bilayer of the modified cell before fusing or being internalized by the modified cell. Similarly, in compositions and methods that include chemical-based transfection of nucleic acid, such as polyethylenimine (PEI) or calcium phosphate-based transfection, the nucleic acid is typically associated with a positively charged transfection reagent to form the recombinant nucleic acid vector that associates with the negatively charged membrane of the modified cell before the complex is internalized by the modified cell. Other means or methods of stably transfecting or genetically modifying cells include electroporation, ballistic delivery, and microinjection. A “polypeptide” as used herein can include part of or an entire protein molecule as well as any posttranslational or other modifications.

A pseudotyping element as used herein can include a “binding polypeptide” that includes one or more polypeptides, typically glycoproteins, that identify and bind the target host cell, and one or more “fusogenic polypeptides” that mediate fusion of the retroviral and target host cell membranes, thereby allowing a retroviral genome to enter the target host cell. The “binding polypeptide” as used herein, can also be referred to as a “T cell and/or NK cell binding polypeptide” or a “target engagement element,” and the “fusogenic polypeptide” can also be referred to as a “fusogenic element”.

A “resting” lymphocyte, such as for example, a resting T cell, is a lymphocyte in the G0 stage of the cell cycle that does not express activation markers such as Ki-67. Resting lymphocytes can include naïve T cells that have never encountered specific antigen and memory T cells that have been altered by a previous encounter with an antigen. A “resting” lymphocyte can also be referred to as a “quiescent” lymphocyte.

As used herein, “lymphodepletion” involves methods that reduce the number of lymphocytes in a subject, for example by administration of a lymphodepletion agent. Lymphodepletion can also be attained by partial body or whole body fractioned radiation therapy. A lymphodepletion agent can be a chemical compound or composition capable of decreasing the number of functional lymphocytes in a mammal when administered to the mammal One example of such an agent is one or more chemotherapeutic agents. Such agents and dosages are known, and can be selected by a treating physician depending on the subject to be treated. Examples of lymphodepletion agents include, but are not limited to, fludarabine, cyclophosphamide, cladribine, denileukin diftitox, alemtuzumab or combinations thereof.

RNA interference (RNAi) is a biological process in which RNA molecules inhibit gene expression or translation by neutralizing targeted RNA molecules. The RNA target may be mRNA, or it may be any other RNA susceptible to functional inhibition by RNAi. As used herein, an “inhibitory RNA molecule” refers to an RNA molecule whose presence within a cell results in RNAi and leads to reduced expression of a transcript to which the inhibitory RNA molecule is targeted. An inhibitory RNA molecule as used herein has a 5′ stem and a 3′ stem that is capable of forming an RNA duplex. The inhibitory RNA molecule can be, for example, a miRNA (either endogenous or artificial) or a shRNA, a precursor of a miRNA (i.e., a Pri-miRNA or Pre-miRNA) or shRNA, or a dsRNA that is either transcribed or introduced directly as an isolated nucleic acid, to a cell or subject.

As used herein, “double stranded RNA” or “dsRNA” or “RNA duplex” refers to RNA molecules that are comprised of two strands. Double-stranded molecules include those comprised of two RNA strands that hybridize to form the duplex RNA structure or a single RNA strand that doubles back on itself to form a duplex structure. Most, but not necessarily all of the bases in the duplex regions are base-paired. The duplex region comprises a sequence complementary to a target RNA. The sequence complementary to a target RNA is an antisense sequence, and is frequently from 18 to 29, from 19 to 29, from 19 to 21, or from 25 to 28 nucleotides long, or in some embodiments between 18, 19, 20, 21, 22, 23, 24, 25 on the low end and 21, 22, 23, 24, 25, 26, 27, 28 29, or 30 on the high end, where a given range always has a low end lower than a high end. Such structures typically include a 5′ stem, a loop, and a 3′ stem connected by a loop which is contiguous with each stem and which is not part of the duplex. The loop comprises, in certain embodiments, at least 3, 4, 5, 6, 7, 8, 9, or 10 nucleotides. In other embodiments the loop comprises from 2 to 40, from 3 to 40, from 3 to 21, or from 19 to 21 nucleotides, or in some embodiments between 2, 3, 4, 5, 6, 7, 8, 9, 10, 11, 12, 13, 14, 15, 16, 17, 18, 19, or 20 on the low end and 10, 11, 12, 13, 14, 15, 16, 17, 18, 19, 20, 25, 30, 35, or 40 on the high end, where a given range always has a low end lower than a high end.

The term “microRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40 nucleotide sequence that flanks a microRNA stem-loop structure. In some instances, the microRNA processing elements are found within a stretch of nucleotide sequences of between 5 and 4,000 nucleotides in length that flank a microRNA stem-loop structure.

The term “linker” when used in reference to a multiplex inhibitory RNA molecule refers to a connecting means that joins two inhibitory RNA molecules.

As used herein, a “recombinant retrovirus” refers to a non-replicable, or “replication incompetent”, retrovirus unless it is explicitly noted as a replicable retrovirus. The terms “recombinant retrovirus” and “recombinant retroviral particle” are used interchangeably herein. Such retrovirus/retroviral particle can be any type of retroviral particle including, for example, gamma retrovirus, and in illustrative embodiments, lentivirus. As is known, such retroviral particles, for example lentiviral particles, typically are formed in packaging cells by transfecting the packing cells with plasmids that include packaging components such as Gag, Pol and Rev, an envelope or pseudotyping plasmid that encodes a pseudotyping element, and a transfer, genomic, or retroviral (e.g., lentiviral) expression vector, which is typically a plasmid on which a gene(s) or other coding sequence of interest is encoded. Accordingly, a retroviral (e.g., lentiviral) expression vector includes sequences (e.g., a 5′ LTR and a 3′ LTR flanking e.g., a psi packaging element and a target heterologous coding sequence) that promote expression and packaging after transfection into a cell. The terms “lentivirus” and “lentiviral particle” are used interchangeably herein.

A “framework” of a miRNA consists of “5′ microRNA flanking sequence” and/or “3′ microRNA flanking sequence” surrounding a miRNA and, in some cases, a loop sequence that separates the stems of a stem-loop structure in a miRNA. In some examples, the “framework” is derived from naturally occurring miRNAs, such as, for example, miR-155. The terms “5′ microRNA flanking sequence” and “5′ arm” are used interchangeably herein. The terms “3′ microRNA flanking sequence” and “3′ arm” are used interchangeably herein.

As used herein, the term “miRNA precursor” refers to an RNA molecule of any length which can be enzymatically processed into an miRNA, such as a primary RNA transcript, a pri-miRNA, or a pre-miRNA.

As used herein, the term “construct” refers to an isolated polypeptide or an isolated polynucleotide encoding a polypeptide. A polynucleotide construct can encode a polypeptide, for example, a lymphoproliferative element. A skilled artisan will understand whether a construct refers to an isolated polynucleotide or an isolated polypeptide depending on the context.

As used herein, “MOI”, refers to Multiplicity of Infection ratio where the MOI is equal to the ratio of the number of virus particles used for infection per number of cells. Functional titering of the number of virus particles can be performed using FACS and reporter expression, as non-limiting examples.

“Peripheral blood mononuclear cells” (PBMCs) include peripheral blood cells having a round nucleus and include lymphocytes (e.g., T cells, NK cells, and B cells) and monocytes. Some blood cell types that are not PBMCs include red blood cells, platelets and granulocytes (i.e., neutrophils, eosinophils, and basophils).

It is to be understood that the present disclosure and the aspects and embodiments provided herein, are not limited to particular examples disclosed, as such may, of course, vary. It is also to be understood that the terminology used herein is for the purpose of disclosing particular examples and embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. When multiple low and multiple high values for ranges are given that overlap, a skilled artisan will recognize that a selected range will include a low value that is less than the high value. All headings in this specification are for the convenience of the reader and are not limiting.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a chimeric antigen receptor” includes a plurality of such chimeric antigen receptors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### DETAILED DESCRIPTION

The present disclosure overcomes prior art challenges by providing improved methods and compositions for modifying and in illustrative embodiments genetically modifying lymphocytes, for example NK cells and in illustrative embodiments, T cells. Some of the methods and compositions herein, provide simplified and more rapid processes for transducing or transfecting lymphocytes that avoid some steps that require specialized devices. Thus, the methods provide an important step toward democratization of cell therapy methods. Illustrative methods and compositions for modifying lymphocytes, for example NK cells and in illustrative embodiments, T cells, are performed in less time than prior methods, and in fact in some embodiments, provide rapid point of care methods. Furthermore, compositions that have many uses, including their use in these improved methods, are provided, including cell formulation compositions that are adapted for subcutaneous administration. Some of these compositions include modified and in illustrative embodiments genetically modified lymphocytes that have improved proliferative and survival qualities, including in in vitro culturing, for example in the absence of growth factors. Such modified and in illustrative embodiments genetically modified lymphocytes will have utility for example, as research tools to better understand factors that influence T cell proliferation and survival, and for commercial production, for example for the production of certain factors, such as growth factors and immunomodulatory agents, that can be harvested and tested or used in commercial products. And such modified and genetically modified lymphocytes having utility in the treatment of cancer.

Illustrative methods and compositions for immune cell therapy herein, include, are compatible with, are effective for, and/or are even adapted for subcutaneous or intramuscular delivery and subcutaneous or intramuscular cell formulations. Some of these delivery methods and cell formulations (i.e., delivery compositions) promote cell aggregation. Such cell aggregation promotes cell proliferation and survival that in some embodiments is further enhanced by the addition of antigen, growth factors and immunomodulatory agents to the cell formulation or to the site of administration of the cell formulation.

Also provided herein are methods and compositions that overcome the challenges associated with the resistance of CAR therapy by CAR-cancer cells such as loss of target antigen availability (e.g., epitope or antigen masking) through genetic modification of malignant cells.

### Illustrative Cell Processing Methods for Genetically Modifying T Cells and/or NK Cells in the Presence of Blood, or a Component Thereof

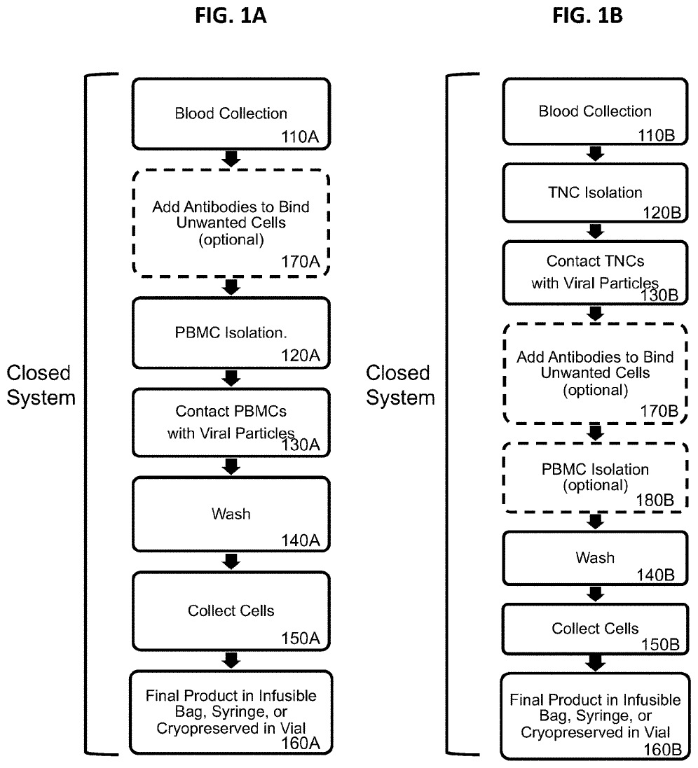

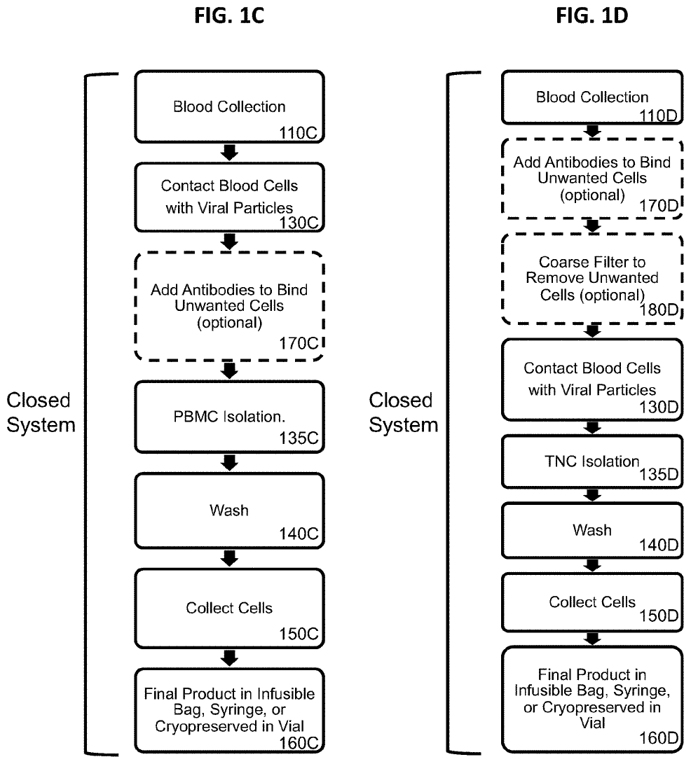

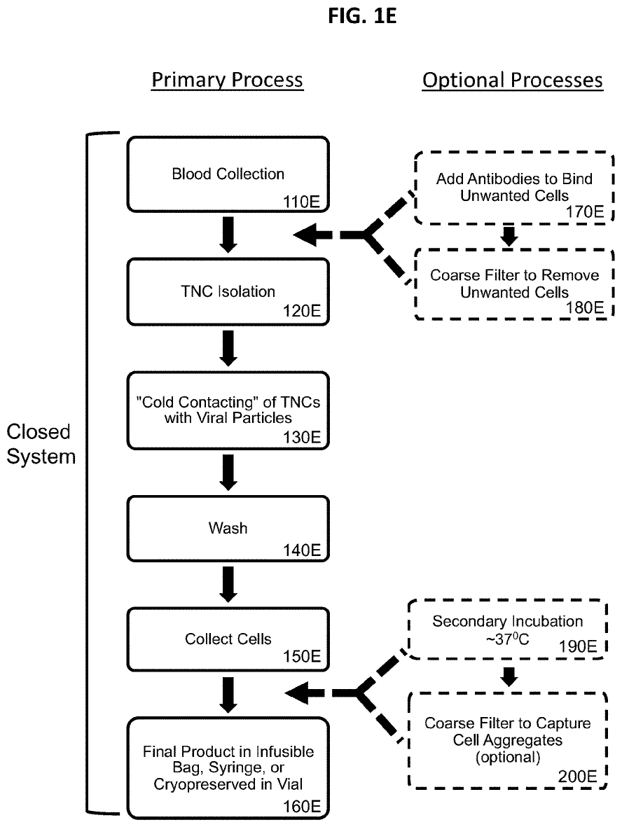

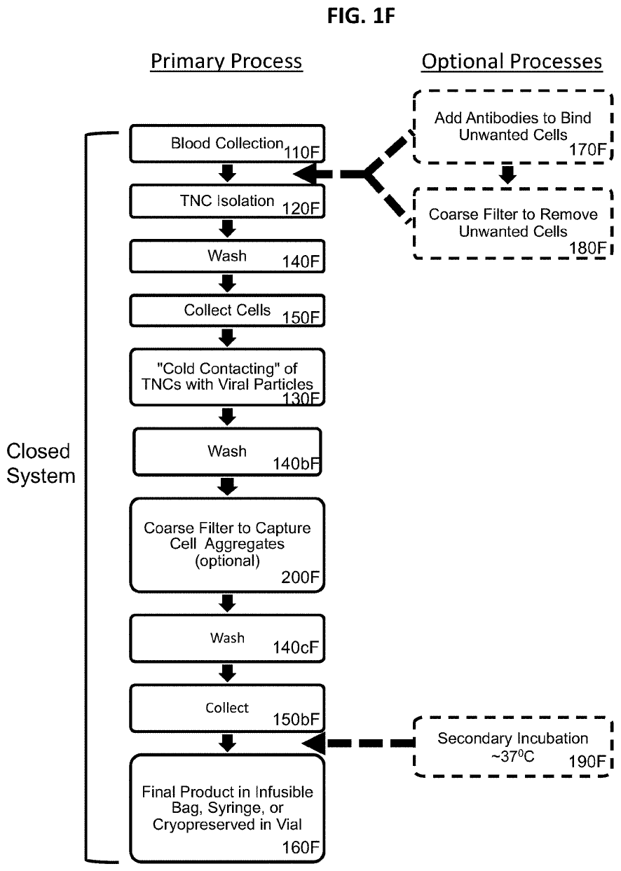

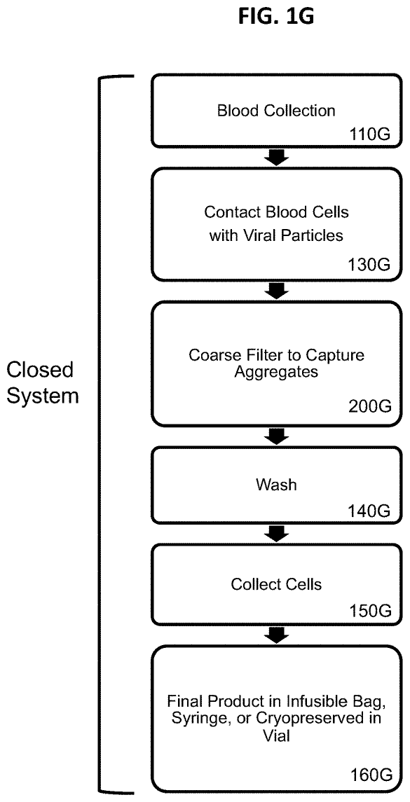

Methods provided herein in illustrative aspects include methods for modifying T cells and/or NK cells, or related methods of making cell formulations, that include contacting blood cells comprising lymphocytes (e.g., NK cells and/or T cells) ex vivo in a reaction mixture, with recombinant vectors such as replication incompetent recombinant retroviral particles, that are or include polynucleotides that encode a CAR. In illustrative embodiments, the reaction mixture includes a T cell activation element, either in solution or on the surface of the recombinant retroviral particles, to facilitate genetic modification of T cells in the reaction mixture. It was demonstrated in the Examples herein that such reaction mixture can include unfractionated whole blood or can include all or many cell types found in whole blood, including total nucleated cells (TNCs), and that in illustrative embodiments, modified T cells are delivered subcutaneously. FIG. 1 provides a number of non-limiting exemplary work flows of such methods.

As shown in FIG. 1, some of the methods provided herein include an optional step where blood is collected (110) from a subject. Blood can be collected or obtained from a subject by any suitable method known in the art as discussed in more detail herein. For example, the blood can be collected by venipuncture, apheresis or any other blood collection method by which a sample of blood is collected. In some embodiments, the volume of blood collected is between 1 and 120 ml. In illustrative embodiments, especially those in which a subject from whom blood is taken has normal levels of NK cells and in illustrative embodiments, T cells, the volume of blood collected is between 1 ml and 25 ml.

It is noteworthy that some embodiments of methods for modifying and in illustrative embodiments genetically modifying provided herein do not include a step of collecting blood from a subject. Regardless of whether blood is collected from a subject, in illustrative method aspects provided herein for modifying lymphocytes (e.g., T cells and/or NK cells), the lymphocytes are contacted with encapsulated nucleic acid vectors (e.g. replication incompetent retroviral particles) in a reaction mixture. In illustrative embodiments, this contacting, and the reaction mixture in which the contacting occurs, takes place within a closed cell processing system, as discussed in more detail herein. Such a closed processing system and method used in some aspects and embodiments of systems and methods provided herein can be any system and method known in the art. As non-limiting examples, the system or method can be a traditional closed cell processing system and method, or a system or method referred to herein as a “more recent” method or system (See e.g., WO2018/136566 and WO2019/055946). In traditional closed cell processing methods that involve genetic modification and/or transductions of lymphocytes ex vivo, especially in methods for autologous cell therapy, many steps occur over days, such as PBMC enrichment(s), washing(s), cell activation, transduction, expansion, collection, and optionally reintroduction. In more recent methods, some of the steps and time involved in this ex vivo cell processing have been reduced (see, e.g., WO2018/136566). In other more recent methods (See FIG. 1A), some of the steps and time involved in this ex vivo cell processing have been further reduced or as with, for example, the ex vivo expansion step, eliminated (see, e.g., WO2019/055946). These more recent methods (as well as the further improved cell processing methods provided herein), furthermore use a rapid ex vivo transduction process, for example that includes no or minimal preactivation (e.g., less than 30, 15, 10, or 5 minutes of contacting lymphocytes such as T cells and/or NK cells with an activation agent before they are contacted with retroviral particles). In certain embodiments of such methods, a T cell and/or NK cell activation element is present in the reaction mixture in which the contacting step occurs. In illustrative embodiments, the T cell and/or NK cell activation element is associated with surfaces of retroviral particles present in the reaction mixture. In illustrative embodiments, such a method that uses rapid ex vivo gene modification without an ex vivo expansion step is used in a rapid point-of-care (rPOC) autologous cell therapy method. However, such more recent methods still involve a PBMC enrichment step/procedure (120A), which typically takes at least around 1 hour within the closed system, followed by cell counting, transfer and media addition, which takes at least around 45 additional minutes before lymphocytes are contacted with retroviral particles to form a transduction reaction mixture (130A). Following the “viral transduction” step, which typically is a contacting step with incubating as discussed in detail herein, lymphocytes are typically washed away from retroviral particles that remain in suspension (140A), for example using a Sepax, and collected by resuspending the PBMCs in a delivery solution (150A) to form a cell formulation typically in an infusion bag for reinfusion, a syringe for injection, or cryopreservation vial for storage (160A). As discussed in further detail herein, traditional PBMC enrichment procedures typically involve ficoll density gradients and centrifugal (e.g., centrifugation) or centripetal (e.g., Sepax) forces or use leukophoresis to enrich PBMCs.

In certain subembodiments, antibodies directed to antigens on the surface of unwanted cells are added to the blood (170A or 170C) or to TNCs (170B) before PBMC isolation, and incubated for an effective period of time to bind to the unwanted cells, as discussed in more detail herein. In certain subembodiments, antibodies directed to antigens on the surface of unwanted cells are added to the blood (170D, 170E, or 170F) before TNC isolation, and incubated for an effective period of time to bind to the unwanted cells, as discussed in more detail herein. The antibodies may be coupled to beads or additional antibodies can be included in the incubation to rosette the unwanted cells to erythrocytes as described in more detail herein. The unwanted cells are then depleted in the PBMC isolation step in which the unwanted cells pellet with the erythrocytes.

As demonstrated in the Examples provided herein, it was surprisingly found that lymphocytes (e.g., T cells and/or NK cells) can be contacted with replication incompetent retroviral particles in a reaction mixture of unfractionated whole blood that optionally contains an anticoagulant, and a significant percentage of the lymphocytes can be modified, genetically modified, and/or transduced. Thus, it was discovered that effective genetic modification of lymphocytes by recombinant retroviral particles can be carried out in the presence of blood components and blood cells in addition to PBMCs and TNCs.

Accordingly, in some embodiments, modification of T cells or NK cells, which is or leads to genetic modification of T cells and/or NK cells, is carried out in a reaction mixture comprising blood components and blood cells in addition to PBMCs, where such genetic modification occurs by contacting T cells and NK cells in the reaction mixture with a recombinant nucleic acid vector, which in illustrative embodiments is a recombinant retroviral particle. In certain illustrative embodiments provided herein (See FIG. 1B, FIG. 1D, FIG. 1E, and FIG. 1F), in place of a PBMC enrichment procedure (e.g., using a density gradient), a cell processing filter or set of filters that enriches lymphocytes over at least one or some other blood cell types (e.g. leukoreduction filter assembly configurable for reverse perfusion with a filter set from which leukocytes can be removed by reverse perfusion), is used (120B, 135D, 120E, and 120F) that also enriches a cell type that is not a PBMC. This step enriches and concentrates lymphocytes, in certain embodiments, before they are contacted with recombinant retroviral particles to form a transduction reaction mixture (130B, 130E and 130F) or in certain embodiments, after they are contacted with recombinant retroviral particles (135D). In certain embodiments, the filter enriches blood cells in addition to PBMCs, for example the filter can enrich TNCs. As shown in FIG. 1B, for example, following the “viral transduction” step, which typically is a contacting step with optional incubating as discussed in detail herein, lymphocytes are typically washed away from retroviral particles that remain in suspension, for example using a Sepax or by passing wash buffer over cells on the leukoreduction filter, and collected by resuspending the PBMCs or TNCs in a delivery solution (150B) to form a cell formulation, with the final cell formulation product typically in an infusion bag for reinfusion, a syringe for injection, or cryopreservation vial for storage (160B).

In illustrative embodiments of methods provided herein, the contacting step with optional incubating of the “viral transduction” step, is performed at temperatures between 32° C. and 42° C., such as at 37° C. In other illustrative embodiments, the contacting step with optional incubating of the “viral transduction” step, is performed at temperatures lower than 37° C., such as between 4° C. and room temperature (referred to herein as the “cold contacting” step) (see FIG. 1E and FIG. 1F). The optional incubating associated with the cold contacting step can be performed for any length of time discussed herein. In illustrative embodiments, the optional incubating associated with the cold contacting step is performed for 1 hour or less. Following the cold contacting and optional incubation step, in some embodiments, the lymphocytes are washed away from retroviral particles that remain on the filter, by passing wash buffer over cells on the leukoreduction filter (140E, 140cF), and collected by resuspending the TNCs in a delivery solution (150E, 150bF) to form a cell formulation, with the final product typically in an infusion bag for reinfusion, a syringe for injection, or cryopreservation vial for storage (160E, 160F). Not to be limited by theory, it is believed that cold contacting TNCs for a period of time, for example 12, 10 8, 6, 4, or 2 hours or less, or in some embodiments, 1 hour or less, with viral particle expressing an activation element on its surface, will lead to binding of the viral particle to T and/or NK cells, but little internalization of the virus. This will also lead to T and/or NK cell aggregates that are cross-linked by viral particles. Furthermore, for embodiments of 4 hours, 2 hours, or 1 hour or less (e.g. 15 minutes to 4 hours, 2 hours, or 1 hour) of contacting, because of the lower temperatures and shorter incubation times, there will be less activation of the cells as compared to cells incubated for longer periods of time and/or temperatures closer to 37° C. It is believed that activation of T and/or NK cells results in their expression of adhesion molecules and binding to the leukoreduction filter, hindering the ability to recover these cells by reverse perfusion of the filter.

In certain embodiments that comprise a cold contacting step, the “viral transduction” step also comprises a secondary incubation (190E, 190F) after the cells have been removed from the leukoreduction filter. In some embodiments the secondary incubation is performed by suspending cells in culture medium such as Complete OpTmizerTM CTS™ T-Cell Expansion Media. In some embodiments, the secondary incubation is performed in the delivery solution. In illustrative embodiments, the secondary incubation is performed in the delivery solution, but lacking any cryopreservation agent. In illustrative embodiments, the secondary incubation is performed at temperatures between 32° C. and 42° C., such as at 37° C. The optional secondary incubation can be performed for any length of time discussed herein. In illustrative embodiments, the optional secondary incubation is performed for less than 4 hours. Not to be limited by theory, it is believed that the secondary incubation of TNCs with viral particles expressing an activation element on its surface will lead to activation of the cells. Activation of T and/or NK cells will cause the cells to aggregate.

Thus, there are at least two mechanisms in the workflows described in FIG. 1 by which T and/or NK cells can form aggregates. (1) surface-bound viral particles cross-link cells, which activity is enhanced at temperatures between 4° C. and room temperature, and (2) activation of T and/or NK cells leads to their aggregation, which is enhanced at temperatures between 32° C. and 42° C. Such aggregates formed by either mechanism under different conditions can be captured by a coarse filter while other debris, singlet cells including lymphocytes, monocytes, and granulocytes, which are approximately 14 μm, and cell aggregates smaller than the pore size of the coarse filter used, pass through into the waste. In some embodiments, a transduction reaction that includes an incubation at temperatures near 37° C., is passed through a coarse filter to capture aggregated T and/or NK cells (200E, 200G). In some embodiments, a transduction reaction that is at or near temperatures between 4° C. and room temperature, is passed through a coarse filter to capture aggregated T and/or NK cells (200F, 200G). Cells on the coarse filter are collected in a delivery solution to form a cell formulation typically in an infusion bag for reinfusion, a syringe for injection, or cryopreservation vial for storage (160F, 160G). In illustrative embodiments wherein a coarse filter is used to collect T and/or NK cell aggregates, the cellular composition of the delivery solution is greater than 40%, 50%, 60%, 70%, 80%, 90% or 95% T cells.

In certain embodiments of reaction mixtures, uses, modified and in illustrative embodiments genetically modified T cell or NK cells, or methods for modifying and/or genetically modifying T cells and/or NK cells, provided herein, a blood sample and thus lymphocytes to be modified, genetically modified, and/or transduced, are not subjected to a PBMC enrichment procedure, before being contacted by recombinant retroviral particles. In some such embodiments, the blood sample, for example an anticoagulated whole blood sample, is applied to a filter, such as a leukoreduction filter assembly, also known as a leukodepletion filter assembly, to obtain total nucleated cells (TNCs) before such TNCs, which comprise lymphocytes from the blood sample, are contacted by recombinant vectors such as recombinant retroviral particles. The leukoreduction filter assembly can include any filter known in the art, for example, filters that collect total nucleated cells (TNCs). In some embodiments, the filter can include a membrane containing polyurethane, cellulose acetate, polyester, combed cotton, PTFE, or GHP. In some embodiments, the leukoreduction filter assembly can include, for example, a HemaTrate™ filter, an Acrodisc™ filter, a Haemonetics® Neo1 filter, a Terumo Imuflex® filter, or any of the leukoreduction filters available from Pall, for example the Leukotrap™ filters, or from Haemonetics®. In some embodiments, the leukoreduction filter is a third or fourth generation or more advanced leukoreduction filter, and can be a depth filter or a screen-type leukoreduction filter (Sharma et al. Asian J Transfus Sci. 2010 January; 4(1): 3-8).

In some embodiments, the volume of blood sample applied to a leukoreduction filter is 2 to 12 ml, 10 to 30 ml, 20 to 50 ml, or 40 to 120 ml (for non-limiting example using a Hematrate filter; Cook Regentec) or 2 to 12 ml (for non-limiting example using an Acrodisc; Pall, AP-4952). In some embodiments, the pore diameter of the filter in a leukoreduction filter assembly is less than 10, 7.5, 5, 4, or 3 μm or from 0.5 to 4 μm. In some embodiments, the leukoreduction filter assembly can collect and/or retain at least 75%, 80%, 90% or 95% of the white blood cells in the blood sample and at least 75%, 80%, 85%, or 90% of the non-leukocyte cells pass through the filter and are not collected. In some embodiments, the leukoreduction filter has an effective filtration area of between 2 cm² and 5 cm² or between 3 cm² and 5 cm². In some embodiments, the coarse filter can be physically attached to the leukoreduction filter assembly. The coarse filter typically has a larger pore diameter larger than the filter in the leukoreduction filter assembly. In some embodiments, the pore diameter of the coarse filter is at least 15 μm and in illustrative embodiments is between 15 and 60 μm. In some embodiments, the coarse filter can be used without using the leukoreduction filter assembly before the contacting step. In addition to being used before the contacting step of methods for modifying and/or genetically modifying T cell and/or NK cells, the coarse filter can be used after the contacting step. In some embodiments, the coarse filter can be used to capture T and/or NK cell aggregates. Such aggregates form when the cells are activated and/or when they are cross-linked by viral particles. In some embodiments, the coarse filter is used to remove singlet blood cells, including neutrophils, which typically pass through the filter. In some embodiments, the coarse filter can be used after the secondary incubation as shown in FIG. 1E. In such embodiments, the filtered cells can be collected and introduced or reintroduced into a subject. As discussed elsewhere herein, it is believed that modified and/or genetically modified cells that are part of an aggregate are advantageously more effective in vivo, especially with subcutaneous administration.

Furthermore, based on the surprising finding discussed above regarding effective genetic modification of T cells and optionally NK cells by retroviral particles even when contacting is performed in unfractionated whole blood (also referred to herein as “whole blood”), provided herein in an illustrative embodiment, is a further simplified method in which lymphocytes are modified, genetically modified, and/or transduced by adding replication incompetent retroviral particles directly to whole blood to form a reaction mixture (130C), and cells in the whole blood are contacted by the replication incompetent retroviral particles for contacting times with optional incubations provided herein. Such a further simplified method in this illustrative embodiment, thus includes no lymphocyte enrichment steps before lymphocytes in whole blood, typically containing an anticoagulant, are contacted with retroviral particles. This further simplified method, like other cell processing methods herein, is typically carried out within a closed cell processing system and can include no or minimal preactivation before lymphocytes are contacted with retroviral particles. In these further simplified methods lymphocytes in whole blood can be contacted with retroviral particles directly in a blood bag. After the contacting step (130C) in such methods, lymphocytes that were contacted with retroviral particles, can be washed and concentrated using a PBMC enrichment procedure (135C). Thus, in such embodiments, no PBMC enrichment procedure and no lymphocyte-enriching filtration is performed before cells in whole blood, and typically comprising an anticoagulant, are contacted with recombinant retroviral particles. However, in the embodiment of FIG. 1C, such a PBMC enrichment method is performed (135C) for example using a Sepax with a ficoll gradient, after the contacting with optional incubation (130C) is carried out. Following the PBMC enrichment, lymphocytes optionally can be washed further away from any retroviral particles that remain unassociated with cells (140C), for example using a Sepax, and collected by resuspending the PBMCs in a delivery solution (150C) to form a cell formulation, with the final product typically in an infusion bag for reinfusion, a syringe for injection, or cryopreservation vial for storage (160C).

In a further illustrative embodiment (FIG. 1D) where a blood sample is not subjected to a PBMC enrichment procedure before recombinant retroviral particles are added to the blood to contact lymphocytes such as T cells and/or NK cells, a PBMC enrichment procedure is not used in any step of the process, even after a contacting step (i.e., step where lymphocytes such as T cell and/or NK cells are contacted by recombinant retroviral particles within the reaction mixture and optionally incubated for any of the contacting and incubating times provided herein). This further simplified method, like other cell processing methods herein, is typically carried out within a closed cell processing system and can include no or minimal preactivation before lymphocytes are contacted with retroviral particles to form a transduction reaction mixture (130D), thus providing a powerful point of care method in some subembodiments. In examples of such further illustrative embodiments, one or more leukoreduction cell processing filtrations (135D), for example using a HemaTrate filter or an Acrodisc filter, can be performed, after the contacting step that includes an optional incubation (130D). Following the leukocyte enriching filtration using the leukoreduction filter, lymphocytes can be optionally washed further away from any retroviral particles that remain (140D), for example by passing PBS with 2% HSA through the filter, and collected (150D), for example using reperfusion with a delivery solution to elute and resuspend TNCs to collect lymphocytes retained on the leukoreduction filter in a cell formulation, with the final product typically a syringe for injection or in an infusion bag for delivery to a subject or a cryopreservation vial for storage (160D).

In a further simplified embodiment (FIG. 1G), the blood sample is not subjected to either a PBMC or TNC enrichment procedure in any step of the process. In this method, lymphocytes in blood are contacted with retroviral particles to form a transduction reaction mixture (130G) and optionally incubated at any temperature between about 4° C. and 42° C. for any of the contacting and incubating times provided herein. The reaction mixture is then passed over a coarse filter to capture aggregated lymphocytes such as T and/or NK cells. Following an optional wash, the cells are collected (150G) for example using reperfusion with a delivery solution to elute and resuspend the cell aggregates, with the final product typically a syringe for injection or in an infusion bag for delivery to a subject or a cryopreservation vial for storage (160G).

As indicated above, the method embodiment workflows shown in FIG. 1, provide modified T cells and/or NK cells suspended in a cell formulation. In methods where PBMCs or lymphocytes are filtered and/or especially where modification, genetic modification, and/or transduction is performed on top of a filter, a delivery solution as provided herein, can be used to elute, resuspend, and collect cells from the filter to form a cell formulation having volumes suitable for administration to a subject, especially subcutaneously or intramuscularly, as provided herein. Such delivery solution can also be used for an optional wash as mentioned above, before the cells are resuspended, eluted and/or otherwise collected for administration. Finally, additional optional steps can be performed in any of the method workflow embodiments of FIG. 1, for example the removal of unwanted cell types (e.g., any cell type other than T cells and/or NK cells), such as B cells and/or cancer cells by negative selection within the closed system as disclosed in more detail herein. EA-rosetting can be performed using antibodies for example, anti-CD19, to complex B cells to erythrocytes (170A, 170B, or 170C) which will pellet away from PBMCs in the density-gradient PBMC isolation step as described in more detail herein. Beads coated with antibodies, for example, to CD19 can be used similarly to complex B cells to beads (170A, 170B, or 170C) which will pellet away from PBMCs in the density-gradient PBMC isolation step. Alternatively, a filtration step can be used. Such a filtration step can be used to remove the cells complexed to beads (180D) or to capture aggregated lymphocytes such as T and/or NK cells that are activated and/or crosslinked by recombinant retroviral particles described herein. In some embodiments, additional wash steps may be performed. In some embodiments, any one or more of the wash steps shown in FIG. 1 or described for a cell process workflow, may be omitted.

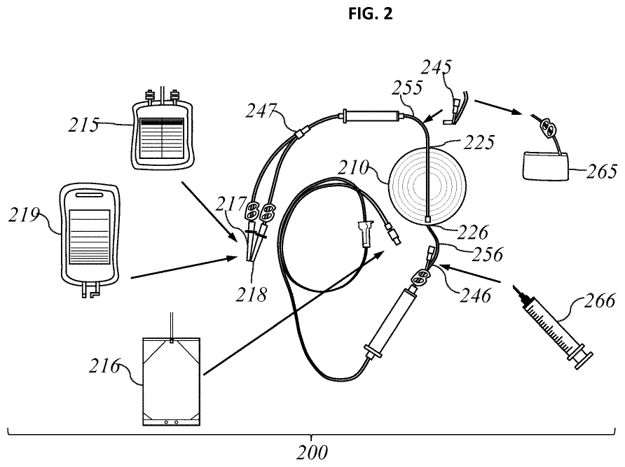

Since a cell filtration process using a leukoreduction filtration assembly like that of FIG. 2 is more rapid than a PBMC enrichment procedure, especially a traditional PBMC enrichment procedure, including density gradient centrifugation (Ficoll-Paque), any of the embodiments of FIG. 1D-G provide an even more rapid method to obtain an enriched preparation of modified, genetically modified, and/or transduced lymphocytes from whole blood, because a time-consuming PBMC enrichment procedure is not performed in any step of such a method, before or after transduction. In illustrative embodiments, the method is performed in a closed cell processing system, thus providing a powerful method for very rapid, relatively simple lymphocyte processing, for example as a point of care CAR-T method that overcomes many of the complications and excessive time limitations of current methods.

As provided in Examples herein, subcutaneous administration has shown surprising results, with increased engraftment of modified and/or genetically modified lymphocytes relative to modified and/or genetically modified lymphocytes introduced through intravenous infusion. This has led to more effective CAR-dependent tumor reduction and elimination in animals. In illustrative embodiments, modified lymphocytes (e.g., T cells and/or NK cells) in a solution are introduced, and in illustrative embodiments reintroduced into a subject by subcutaneous administration, delivery, or injection. In some examples of these embodiments that involve contacting lymphocytes in reaction mixtures with retroviral particles such as those exemplified in FIG. 1, including illustrative embodiments that include at least some other blood components not typically present after the lymphocytes are isolated in a PBMC enrichment procedure, resulting cell formulations, which are separate aspects provided herein, are optionally administered (e.g., readministered) into a subject. In illustrative embodiments, (FIG. 1D) where a PBMC enrichment procedure is not used after lymphocytes are contacted with retroviral particles, cell formulations produced there can be reintroduced back into a subject using subcutaneous or intramuscular administration. Thus, as discussed in more detail herein, some aspects provided herein are cell formulations, as well as delivery solutions (i.e., excipients) for making such cell formulations, that are compatible with, in illustrative embodiments effective for, and in further illustrative embodiments adapted for subcutaneous delivery. Not to be limited by theory, it is believed that the presence of additional blood cells, especially neutrophils, in a process that only uses cell processing filters to concentrate and/or wash lymphocytes, such as HemaTrate filters, renders the cell preparations more amenable to subcutaneous delivery to avoid some additional risks present if these other blood cell types, especially neutrophils or aggregated T cells, are infused directly back into the blood of a patient. For example, a subcutaneous formulation of retrovirus reconstituted with total nucleated cells on a lymphoreduction filter may contain, in addition to lymphocytes, neutrophils (or more generally granulocytes). In illustrative embodiments, the cell formulation comprises neutrophils, B cells, monocytes, red blood cells, basophils, eosinophils, and/or macrophages together with modified T cells (CAR-T cells) and/or NK cells (CAR-NK cells). A subcutaneous or intramuscular formulation and administration are advantageous over intravenous formulation and administration because a formulation (suspension) of retrovirus reconstituted with lymphocytes may further comprise cellular aggregates and express adhesion receptors that may introduce pulmonary congestion with intravenous delivery.

Methods for subcutaneous administration are well known in the art and typically involve administration into the fat layer under the skin. It should be noted that it is contemplated that any embodiment herein that involves subcutaneous delivery, can instead be intramuscular delivery, which is delivery into the muscle, intradermal, or intratumoral delivery. In some embodiments, subcutaneous administrations can be performed in the upper thigh, upper arm, abdomen, or upper buttocks of a subject. Subcutaneous administration is distinguishable from intraperitoneal administration, which penetrates through the fatty layer used in subcutaneous administration and delivers a formulation or drug into the peritoneum of the subject.

In such embodiments, where cells are introduced or reintroduced (also referred to herein as delivered) into a subject by subcutaneous administration in larger volumes of excipient (also referred to herein as subcutaneous injection or delivery), to facilitate such subcutaneous administration, hyaluronidase may be added to the isolated modified, genetically modified, and/or transduced lymphocyte preparation that contains the lymphocytes that have been contacted with a recombinant retrovirus, or injected subcutaneously at or near the same location of sequential delivery of the isolated modified, genetically modified, and/or transduced lymphocyte preparation. In illustrative embodiments, an effective amount of hyaluronidase is used, particularly in embodiments where more than 1 or 2 ml (e.g., 2-1,000 ml, 2-500 ml, 2-100 ml, 2-50 ml, 2-10 ml, 2-5 ml, 5-1,000 ml, 5-500 ml, 5-100 ml, 5-50 ml, or 5-10 ml) of a cell formulation of lymphocytes that have been contacted with retroviral particles, e.g., of a cell formulation comprising modified NK cells, and in illustrative embodiments T cells, are to be reintroduced subcutaneously into a subject. Not to be limited by theory, hyaluronidase, for example recombinant human hyaluronidase, facilitates the dispersion and absorption of other injected therapeutics by enabling large volume subcutaneous delivery, especially beyond the typically administered 2 ml or less volume, and potentially enhances pharmacokinetic profiles of a co-injected therapeutic (See e.g., Bookbinder LH, et al. “A recombinant human enzyme for enhanced interstitial transport of therapeutics.” J. Control Release (2006) Aug. 28; 114(2): 230-41. Epub 2006 Jun. 7, incorporated by reference herein, in its entirety; and Frost, GI, et al. “Recombinant human hyaluronidase (rHuPH20): an enabling platform for subcutaneous drug and fluid administration.” Expert Opinion Drug Delivery (2007) July; 4(4); 427-440, incorporated by reference herein, in its entirety. Dispersion of fluid in the cell mixture may be facilitated with larger volumes while minimizing vascular compression at the injection site. Hyaluronidase (e.g., recombination human hyaluronidase PH20 enzyme (rHuPH20), or Hylenex® 150 USP Units), is available from Halozyme Therapeutics, Inc. (San Diego, CA). In some embodiments, between 50 and 5000; or between 1,000 and 3,000 units/ml of rHuPH20 can be delivered together with the modified, genetically modified, and/or transduced lymphocytes in 1 to 50 ml, 2 to 25 ml, 2 to 20 ml, 2 to 10 ml, 2 to 5 ml, 2 to 4 ml, 2.5 to 25 ml, 2.5 to 20 ml, 2.5 to 10 ml, 2.5 to 5 ml, 5 to 20 ml, or 5 to 10 ml for example, or such delivery of hyaluronidase and lymphocytes can be sequential. Additional hyaluronidase enzymes for example, can be found in U.S. Pat. No. 7,767,429, incorporated by reference herein, in its entirety.

FIG. 2 provides a non-limiting illustrative example of a cell processing leukoreduction filtration assembly (200) that enriches nucleated cells that can be used as the leukoreduction filter in the methods of FIG. 1. The illustrative leukoreduction filtration assembly (200), which in illustrative embodiments is a single-use filtration assembly, comprises a leukocyte depletion media (e.g. filter set) within a filter enclosure (210), that has an inlet (225), and an outlet (226), and a configuration of bags, valves and/or channels/tubes that provide the ability to concentrate, enrich, wash and collect retained white blood cells or nucleated blood cells using perfusion and reverse perfusion (see e.g. EP2602315A1, incorporated by reference herein, in its entirety). In an illustrative embodiment, the leukoreduction filtration assembly (200) is a commercially available HemaTrate filter (Cook Regenetec, Indianapolis, IN). Leukoreduction filtration assemblies can be used, to concentrate total nucleated cells (TNC) including granulocytes, which are removed in PBMC enrichment procedures in a closed cell processing system. Since a filter assembly comprising leukocyte depletion media of EP2602315A1 such as a HemaTrate filter and the illustrative leukoreduction filter assembly of FIG. 2 do not remove granulocytes, they are not considered PBMC enrichment assemblies or filters herein, and methods that incorporate them are not considered PBMC enrichment procedures or steps herein.

The leukoreduction filter assembly (200) of FIG. 2 is a single-use sterile assembly that includes various tubes and valves, typically needle-free valves, that allow isolation of white blood cells from whole blood and blood cell preparations that include leukocytes, as well as rapid washing and concentrating of white blood cells. In this illustrative assembly, a reaction mixture collection container (215), for example a 500 ml PVC bag containing about 120 ml of a transduction/contacting reaction mixture comprising whole blood, an anticoagulant, and retroviral particles is connected to the assembly (200) at a first assembly opening (217) of an inlet tubing (255), after the reaction mixture is subjected to a contacting step with optional incubation, as disclosed in detail herein. Lymphocytes, including some modified T cells and/or NK cells with associated retroviral particles, and some that could be genetically modified at this point, as well as other blood cells and components in the whole blood reaction mixture as well as the anticoagulant enter the inlet tubing (255) through the first assembly opening (217) by gravitational force when a clamp on the first inlet tubing (255) is released. The modified and/or genetically modified T cells and/or NK cells pass through an inlet valve (247) and a collection valve (245), to enter a filter enclosure (210) through a filter enclosure inlet (225) to contact a leukoreduction IV filter set (e.g., SKU J1472A Jorgensen Labs) within the filter enclosure (210). Nucleated blood cells including leukocytes are retained by the filter, but other blood components pass through the filter and out the filter enclosure outlet (226) into the outlet tubing (256), then through an outlet valve (246) and are collected in a waste collection bag (216), which for example can be a 2L PVC waste collection bag.

An optional buffer wash step can be performed by switching inlet valve (247) to a wash position. In this optional wash step, a buffer container (219), for example a 500 ml saline wash bag, is connected to a second assembly opening (218) of inlet tubing (255). The buffer moves into the inlet tubing (255) through the second assembly opening (218) by gravitational force when a clamp on the inlet tubing (255) is released. The buffer passes through inlet valve (247) and collection valve (245), to enter filter enclosure (210) through the filter enclosure inlet (225) and passes through the leukoreduction filter set within the filter enclosure (210) to rinse the cells retained on the filter. The buffer moves out the filter enclosure outlet (226) into the outlet tubing (256), then through an outlet valve (246) and is collected in a waste collection bag (216), which can be the same waste collection bag as used to collect reaction mixture components that passed through the filter in the previous step, or a new waste collection bag swapped in place of the first waste collection bag before the buffer was allowed to enter the second assembly opening (218). The optional wash step can be optionally performed multiple times by repeating the above process with additional buffer. Furthermore, in some embodiments the optional wash step is performed at least in part, using the elution/delivery solution.

Once the entire or substantially the entire volume of the reaction mixture in the reaction mixture collection container (215) passes over the filter (210), and the optional washing step(s) is optionally performed, a reverse perfusion process is initiated to move fluid in an opposite direction in the assembly (200) to collect lymphocytes retained on the filter set within the filter enclosure (210). Illustrative embodiments of leukoreduction filter assemblies herein are adaptable for reperfusion. Before initiating the reverse perfusion process in the illustrative assembly (200), the outlet valve (246) is switched to a reperfusion position and the collection valve (245) is switched to a collection position. To initiate reperfusion, a delivery solution, which in some embodiments can be a buffer (e.g., PBS) that can have additional components as provided herein, and can be an elution solution, in syringe (266), which for example can be a 25 ml syringe, is passed into outlet tubing (256) by injection using syringe (266). The delivery solution then enters the filter enclosure (210) through the filter enclosure outlet (226) and suspends lymphocytes retained on the filter set into a cell formulation and moves the cell formulation out of the filter enclosure (210) through the filter enclosure inlet (225) and into the inlet tubing (255). Then the cell formulation that contains modified lymphocytes, including some T cells and/or NK cells with associated retroviral particles, some of which could be genetically modified and/or transduced at this point, are collected in a cell sample collection bag (265), which for example can be a 25 ml cryopreservation bag, after the pass through the collection valve (245). The collected cell formulation optionally can then be administered to a subject, such as through subcutaneous administration.

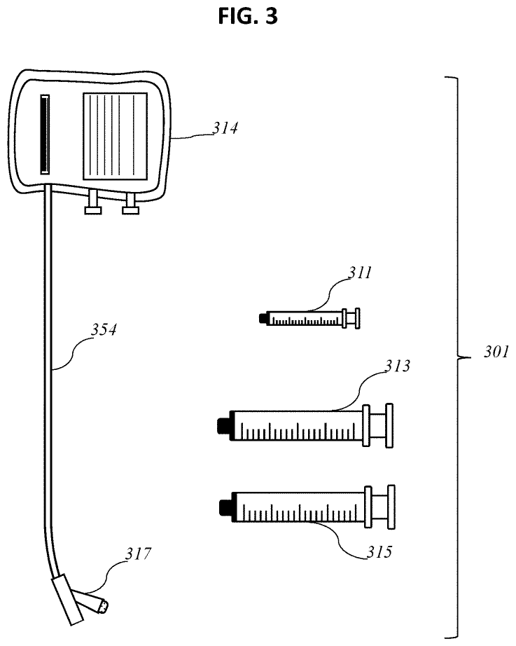

The transduction assembly (301) of FIG. 3 is a single-use sterile assembly that includes tubes and valves, typically needle-free valves, that allow modification and transduction of cells in whole blood comprising lymphocytes, and typically also comprising an anticoagulant such as heparin, for example at 50 U/ml. In certain embodiments, the whole blood comprising lymphocytes does not comprise red blood cells, which can be removed by known methods after blood collection. In illustrative embodiments, the whole blood comprising lymphocytes does comprise red blood cells. In illustrative embodiments, the whole blood comprising lymphocytes does comprise red blood cells. Transduction assemblies, such as the one in FIG. 3, themselves, optionally containing any of the reaction mixtures disclosed herein, form aspects and embodiments of the current disclosure and can also be used in any of the aspects and methods provided herein that include a contacting step. In the illustrative assembly of FIG. 3, a vector container (311) containing between 0.5 ml and 20 ml vector, between 0.5 and 2.5 ml, and in illustrative embodiments between 2 ml and 5 ml vector in a solution, for example PBS containing 4% lactose, is attached to the first assembly opening (317) of tubing (354). In illustrative embodiments, the first assembly opening (317) is a sterile needle-free valve connector. Optionally, the volume of vector is chosen based on the titer of vector to be delivered, for example, in the case of replication incompetent retroviral particles (“RIPs”), using any of the methods provided herein for determining titer, including, for example, by determination of dimming units. A force, such as a positive pressure, is then applied to transfer the contents of the vector container (311) through the first assembly opening (317) into the tubing (354) and then into the incubation bag (314). Optionally, the first assembly opening (317) is located directly on the incubation bag (314) and there is no tubing (354). The incubation bag (314) can have a capacity of 10 ml to 200 ml, for example 15 ml to 100 ml, 15 ml to 50 ml, or 15 ml to 30 ml, and can optionally be a bag that allows gas exchange such as a blood bag. The vector container (311) optionally also contains a volume of air, for example, a volume of air sufficient to help push the contents of the vector container (311) through the tubing (354) and into the incubation bag (314). In any of the steps herein, the containers whose contents are transferring can be positioned such that air bubbles in the containers are at the top of the container during the transferring. For any step herein wherein a force is applied, the force can be a positive pressure or a negative pressure that is generated using any method known in the art, for example, gravity feed, manual force, such as by depressing or pulling the plunger of a syringe, peristaltic pumps, and/or syringe pumps. In some embodiments, the contents are transferred using methods other than a gravity feed.

After transferring the contents of the vector container (311) into the first assembly opening (317), the vector container (311) is detached from the first assembly opening (317). A whole blood container (313) containing 5-100 ml, 5-50 ml, 5-25 ml, and in illustrative embodiments 5-20 ml or 5-15 ml whole blood, and optionally containing an additional volume of air, for example, a volume of air sufficient to help push the contents of the whole blood container through the tubing (354) and into the incubation bag (314), is attached to the first assembly opening (317). A positive force is applied to transfer the whole blood through the first assembly opening (317) through the tubing (354) and into the incubation bag (314) to form a reaction mixture that includes the whole blood and the vector. Optionally, the whole blood in the whole blood container (313) is collected from a subject into the whole blood container (313), and optionally subjected to a red blood cell depletion procedure, before the whole blood container (313) is connected to the first assembly opening (317).

After the reaction mixture is formed, the incubation bag (314) is then incubated for 15 minutes to 12 hours, which in illustrative embodiments can be 2 to 8 hours, or any of the times provided herein for contacting and optional incubating of lymphocytes with a vector. The incubation is typically carried out at 37° C. and 5% CO₂, with one or more optional mixing steps at any time or throughout the incubation. The optional mixing steps can be performed for example at the beginning and can include massaging the incubation bag (314) manually or agitating the incubation bag (314) through rocking or rotating. After its contents are transferred into the transduction assembly (301), the whole blood container (313) is detached from the first assembly opening (317) and the reaction mixture collection container (315) is attached to the first assembly opening (317). A force is then applied to transfer the reaction mixture from the incubation bag (314) through the tubing (354) and first assembly opening (317) and into the reaction mixture collection container (315), for example using negative pressure from the reaction mixture collection container (315).

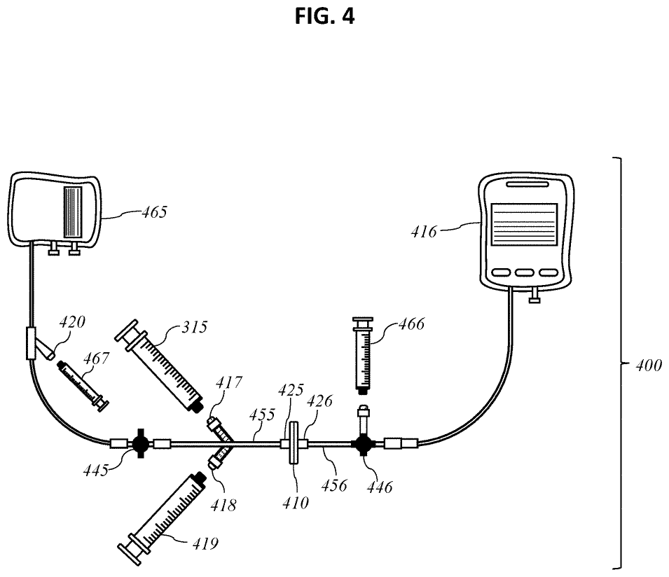

The leukoreduction filter assembly (400) of FIG. 4 is a single-use sterile assembly that includes various tubes and valves, typically needle-free valves, that allow isolation of total nucleated cells from whole blood and blood cell preparations that include leukocytes, as well as rapid washing and concentrating of total nucleated cells. Leukoreduction filter assemblies, such as the one in FIG. 4, may themselves form aspects and embodiments of the current disclosure, optionally including any of the solutions (e.g., reaction mixtures) comprising modified lymphocytes provided herein, and can also be used in any of the aspects and methods provided herein that include a reaction mixture. In this illustrative assembly, a reaction mixture collection container (315), for example a 30 ml syringe containing about 5-25, 5-20, 7.5-20, or 10-15 ml of a transduction/contacting reaction mixture comprising whole blood and vector, and in illustrative embodiments an anticoagulant, is connected to the assembly (400) at a first assembly opening (417) of an inlet tubing (455), after the reaction mixture is subjected to a contacting step with optional incubation, as disclosed in detail above for FIG. 3 and elsewhere herein. In illustrative embodiments, the first assembly opening (417) is a sterile needle-free valve connector. The reaction mixture collection container (315) optionally contains a volume of air, for example, a volume of air that is sufficient to help assure complete movement of the contents of the reaction mixture collection container (315) through the inlet tubing (455) through the filter enclosure inlet and onto the filter in the filter enclosure (410). In some embodiments, the reaction mixture collection container (315) and the inlet tubing (455) at the time of transfer is between 80° and 90°. In illustrative embodiments, the angle between the reaction mixture collection container (315) and the inlet tubing (455) at the time of transfer is less than or about 80°, 75°, 70°, 65°, 60°, 55°, 50°, or 45°, which in illustrative embodiments can be about 45°. In illustrative embodiments, the cells are not moved across any junction with a greater than 70°, 75°, or 80° angle in the leukoreduction filter assembly (400).

Lymphocytes, including, for example, modified T cells and/or NK cells with associated vector, and/or genetically modified T cells and/or NK cells, as well as other blood cells (e.g., neutrophils) and components in the reaction mixture such as anticoagulant are transferred through the first assembly opening (417) and into the inlet tubing (455) by application of a force to the contents of the reaction mixture collection container (315). In some embodiments of any of the methods used to transfer the contents, including, for example, to transfer the modified cells, such as modified T cells and/or NK cells from the reaction mixture collection container (315) onto the filter in the filter enclosure (410), the flow rate is less than or about 5 ml/min, 4 ml/min, 3 ml/min, 2.5 ml/min, 2 ml/min, 1 ml/min, 0.75 ml/min, 0.5 ml/min, or 0.25 ml/min, which in illustrative embodiments can be between 0.25 ml/min and 5 ml/min, 0.5 ml/min and 2.5 ml/min, or 0.75 ml/min and 1.5 ml/min. Additionally, the containers whose contents are being transferred can be positioned such that air bubbles in the containers are at the top of the container during the transferring. The modified and/or genetically modified cells pass through the first assembly opening (417) to enter the inlet tubing (455) and then pass through a filter enclosure inlet (425) into a filter enclosure (410) to contact a leukoreduction IV filter (e.g., Acrodisc WBC 25 mm PSF (Product ID: AP-4952)) within the filter enclosure (410). The leukoreduction IV filter has an effective filtration area of 1 to 10 cm², and in illustrative embodiments 3 to 5 cm². Nucleated blood cells, including leukocytes, for example the modified T cells and/or NK cells, are retained by the filter in the filter enclosure (410), while other blood components and components in the reaction mixture pass through the filter and out the filter enclosure outlet (426) into the outlet tubing (456), then through an outlet valve (446) and are collected in a waste collection bag (416), which for example can be a 2 L PVC waste collection bag.

An optional buffer wash step can be performed by attaching a buffer container (419), for example a syringe containing a volume 0.25-fold to 2-fold the volume of the reaction mixture, which in illustrative embodiments can be 5 ml to 30 ml, 5 to 25 ml, 5 to 20 ml, or 5 to 15 buffer, for example, about 10 ml buffer, to a second assembly opening (418) of the inlet tubing (455). In illustrative embodiments, the second assembly opening (418) is a sterile needle-free valve connector. A force can be applied to transfer the buffer through the second assembly opening (418) into and through the inlet tubing (455) to enter the filter enclosure (410) through the filter enclosure inlet (425) and through the leukoreduction filter within the filter enclosure (410) to rinse the cells retained on the filter. In some embodiments, the flow rate of buffer over the filter enclosure (410) is less than or about 5 ml/min, 4 ml/min, 3 ml/min, 2.5 ml/min, 2 ml/min, 1 ml/min, 0.75 ml/min, 0.5 ml/min, or 0.25 ml/min, which in illustrative embodiments can be between 0.25 ml/min and 5 ml/min, 0.5 ml/min and 2.5 ml/min, or 0.75 ml/min and 1.5 ml/min. The buffer and remaining blood components and components in the reaction mixture not retained on the filter pass through the filter and out the filter enclosure outlet (426) into the outlet tubing (456) then through an outlet valve (446) and are collected in a waste collection bag (416), which can be the same waste collection bag as used to collect reaction mixture components that passed through the filter in the previous step, or a new waste collection bag swapped in place of the first waste collection bag before the buffer was allowed to enter the second assembly opening (418). The optional wash step can be optionally performed multiple times by repeating the above process with additional buffer, using the same or different buffer containers in multiple washes. Furthermore, in some embodiments an optional wash step is performed at least in part, using the elution/delivery solution. In some embodiments, when different buffer containers are used, the same or different buffers can be used in different washes. In illustrative embodiments, the optional wash step is performed once. In some embodiments, with or without the optional wash step, by using filtration, in illustrative embodiments a leukoreduction filter, at least at least 75%, 80%, 85%, 86%, 87%, 88%, 89%, 90%, 91%, 92%, 93%, 94%, 95%, 96%, 97%, 98%, 99%, 99.5%, or 99.9% of the unbound gene vector (e.g. gene vector particles), and in illustrative embodiments RIPs not associated with the population of modified lymphocytes, are removed.

Once the entire or substantially the entire volume of the reaction mixture in the reaction mixture collection container (315) passes over the filter (410), and the optional washing step(s) is optionally performed, a reverse perfusion process is initiated by applying a force to move fluid in an opposite direction in the leukoreduction filter assembly (400) to collect lymphocytes retained on the filter set within the filter enclosure (410), with an optional step of positioning the leukoreduction filter assembly (400) such that the filter enclosure inlet (425) is pointing down and gravity facilitates the elution. Illustrative embodiments of leukoreduction filter assemblies herein are adaptable for reverse perfusion (reperfusion). Before initiating the reverse perfusion process in the illustrative leukoreduction filter assembly (400), the outlet valve (446) is switched to a reperfusion position and the collection valve (445) is switched to a collection position. To initiate reperfusion, a volume of elution solution, for example a delivery solution, as disclosed herein, which in some embodiments can be, for example, human serum albumin in saline or Plasmalyte, that can have additional components as provided herein, in a syringe (466) is passed into outlet tubing (456) by injection through the outlet valve (446), for example by depressing a plunger of the syringe (466). In some embodiments, the volume of delivery solution or elution solution can be for example, between 0.5 ml and 20 ml, 1 ml and 10 ml or 2 ml and 7 ml. The delivery solution is typically transferred into the outlet tubing (456) quickly to aid in elution, for example, at a flow rate of at least or about 5 ml/min, 10 ml/min, 20 ml/min, or 60 ml/min or by immediately plunging the plunger of the syringe (466). The delivery solution then enters the filter enclosure (410) through the filter enclosure outlet (426) and suspends lymphocytes retained on the filter set into a cell formulation and moves the cell formulation out of the filter enclosure (410) through the filter enclosure inlet (425) and into the inlet tubing (455). Then the cell formulation that contains modified lymphocytes, including some T cells and/or NK cells with associated vector, some of which could be genetically modified and/or transduced at this point, are collected in a cell sample collection bag (465), which for example can have a maximum volume, capacity, or volume capacity of a 5 to 50 ml, 10 to 40 ml, 15 to 35 ml or about 25 ml and can be a cryopreservation bag, after passing through the collection valve (445). The collected cell formulation optionally can then be administered to a subject, such as through subcutaneous administration or combined or supplemented with other components disclosed herein. The collected cell formulation is typically transferred to a syringe before administration. For example, a cell sample collection syringe (467) can be attached to a third assembly opening (420). In illustrative embodiments, the third assembly opening (418) is a sterile needle-free valve connector. A force is then applied to transfer the collected cell formulation from the cell sample collection bag (465) through the third assembly opening (420) and into the cell sample collection syringe (467), for example using negative pressure from the cell sample collection syringe (467).

### Self-Driving Car Methods and Compositions

Provided herein in certain aspects are polynucleotides referred to herein as “self-driving CARs” that encode a membrane-bound lymphoproliferative element whose expression in a T cell or NK cell is under the control of an inducible promoter that is induced by the binding of an antigen to an extracellular binding pair member polypeptide that is expressed by the T cell or NK cell and is functionally linked to a intracellular activating domain, for example a CD3 zeta intracellular activating domain or any of the intracellular activating domains disclosed elsewhere herein. In illustrative embodiments, such a binding pair member polypeptide is a CAR. In other embodiments, such a binding pair member polypeptide is a TCR. Thus, in certain embodiments, provided herein are polynucleotides that include an inducible promoter operably linked to a nucleic acid encoding a membrane-bound lymphoproliferative element, that is induced by CAR-binding to its target. Expression of the lymphoproliferative element can induce proliferation of the T cell or NK cell. Provided herein in certain aspects are genetically modified or transduced T cells referred to herein as “self-driving CAR-T cells” that include a self-driving CAR. Any of the embodiments that include a self-driving CAR-T cell could include a “self-driving CAR NK cell,” which is a genetically modified or transduced NK cell that includes a self-driving CAR. In some embodiments, the self-driving CAR NK cell is present in addition to the self-driving CAR-T cell. In other embodiments, the self-driving CAR NK cell is present instead of the self-driving CAR-T cell. Various embodiments that include a self-driving CAR are disclosed in the Exemplary Embodiments section herein and can be combined with any of the embodiments or details of this section.

Accordingly, provided herein in certain embodiments, is an isolated polynucleotide that includes a first sequence comprising one or more first transcriptional units operably linked to an inducible promoter inducible in at least one of a T cell or an NK cell, wherein at least one of the one or more first transcriptional units comprises a first polynucleotide sequence encoding a first polypeptide comprising a lymphoproliferative element and in illustrative embodiments, a second transcriptional unit encoding a chimeric antigen receptor (CAR), wherein the CAR comprises an antigen-specific targeting region (ASTR), a transmembrane domain, and an intracellular activating domain In certain illustrative embodiments, the lymphoproliferative element is constitutively active in at least one of a T cell or an NK cell, and the lymphoproliferative element comprises a transmembrane domain. In illustrative embodiments, the one or more first transcriptional units of a self-driving CAR does not encode a polypeptide that comprises a signal peptide sequence comprising a signal peptidase cleavage site, or other sequence that would result in the encoded polypeptide, once expressed, being secreted or otherwise released from the T or NK cell.

Provided herein in another self-driving CAR embodiment, is an isolated polynucleotide that includes a first sequence in a reverse orientation comprising one or more first transcriptional units operably linked to an inducible promoter inducible in at least one of a T cell or an NK cell, and further includes a second sequence in a forward orientation comprising one or more second transcriptional units operably linked to a constitutive T cell or NK cell promoter, wherein the number of nucleotides between the 5′ end of the one or more first transcriptional units and the 5′ end of the one or more second transcriptional units is less than the number of nucleotides between the 3′ end of the one or more first transcriptional units and the 3′ end of the one or more second transcriptional units, wherein at least one of the one or more first transcriptional units encodes a lymphoproliferative element, and wherein at least one of the one or more second transcriptional units encodes a chimeric antigen receptor (CAR), wherein the CAR comprises an antigen-specific targeting region (ASTR), a transmembrane domain, and an intracellular activating domain. The distances between the 5′ end of the one or more first transcriptional units and the 5′ or 3′ end of the one or more second transcriptional units can be measured, for example, as the number of nucleotides between the 5′ nucleotide of the one or more first transcriptional units and the 5′ or 3′ nucleotide of the one or more second transcriptional units. In some embodiments, the one or more first transcriptional units and the one or more second transcriptional units are transcribed divergently, and such transcriptional units are said to be arrange divergently, i.e., in opposite directions, wherein the 3′ ends of the one or more first and one or more second transcriptional units are farther away from each other than the 5′ ends of the one or more first and one or more second transcriptional units. The polynucleotides or vectors containing two transcriptional units, i.e., a first and second one or more transcriptional units, can be referred to herein as bicistronic polynucleotides or vectors. A divergent bicistronic polynucleotide may encode 2, 3, 4 or more polypeptides and/or inhibitory RNAs.

In another embodiment, provided herein are genetically modified lymphocyte(s), in illustrative embodiments genetically modified T cell(s) and/or NK cell(s), that have been transduced and/or genetically modified with a polynucleotide disclosed above. In yet another embodiment provided herein, is a use of a replication incompetent recombinant retroviral particle(s) in the manufacture of a kit for genetically modifying and/or transducing a lymphocyte, in illustrative embodiments a T cell and/or NK cell of a subject, wherein the use of the kit comprises transducing and/or genetically modifying the T cell or NK cell with a polynucleotide disclosed above, in vivo or in vitro. In another embodiments, provided herein are methods for administering a genetically modified lymphocyte to a subject, wherein the genetically modified lymphocyte is produced by transducing and/or genetically modifying lymphocytes with a polynucleotide disclosed in this Self-Driving CAR section. In some embodiments of any of the aspects herein, the administration of the genetically modified lymphocytes or the replication incompetent retroviral particles, can be performed by intravenous injection, intraperitoneal administration, subcutaneous administration, or intramuscular administration. In some embodiments, the modified lymphocytes introduced into the subject can be allogeneic lymphocytes. In such embodiments, the lymphocytes are from a different person, and the lymphocytes from the subject are not modified. In some embodiments, no blood is collected from the subject to harvest lymphocytes.

In illustrative embodiments of any of the composition and method embodiments for transducing lymphocytes with a self-driving CAR, the polynucleotide can include a constitutive T cell or NK cell promoter. Constitutive T cell or NK cell promoters that constitutively express a polynucleotide in a T cell or NK cell are known in the art and disclosed elsewhere herein. In some embodiments, a transcriptional unit is a constitutive expression unit or construct, which in illustrative embodiments of self-driving CAR embodiments, encodes a CAR. In some embodiments, a constitutive expression construct is or is part of a recombinant expression vector described herein.

In some embodiments, a transcriptional unit is an inducible expression unit or construct, which in illustrative embodiments of self-driving CAR embodiments, can encode a lymphoproliferative element. An inducible expression construct can comprise regulatory sequences, such as transcription and translation initiation and termination codons. In some embodiments, such regulatory sequences are specific to the type of cell into which the inducible promoter is to be introduced, i.e., a T cell and/or an NK cell. An inducible expression construct can comprise a native or non-native promoter operably linked to a nucleotide sequence of interest. In some embodiments, the inducible or activatable promoter can be an NFAT-responsive, ATF2-responsive, AP-1 responsive, or NF-κB-responsive promoter. Other promoters that are induced upon T cell activation and can be used as inducible promoters in embodiments herein, especially embodiments for self-driving CARs, include an IL-2, IFNg, CD25, CD40L, CD69, CD107a, TNF, VLA1, or LFA1 promoter, or a functional and inducible fragment of any of these promoters. As discussed herein, such inducibility can result from the presence of one or more NFAT-binding elements.

In illustrative embodiments of any of the composition and method embodiments for transducing lymphocytes with a self-driving CAR, the first sequence can be in the reverse orientation and the second sequence can be in the forward orientation. The orientations of the first and second sequences are relative to the 5′ to 3′ orientation established by the 5′ LTR and the 3′ LTR of the polynucleotide when present in a recombinant retroviral particle capable of genetically modifying a T cell or NK cell. Thus, a sequence, for example a transcriptional unit, a promoter, a coding sequence, a miRNA, whose 5′ end is closer to the 5′ LTR than its 3′ end is to the 5′ LTR, is in forward orientation and a sequence whose 3′ end is closer to the 5′ LTR than its 5′ end is to the 5′ LTR, is in reverse orientation. The distance between either end of a sequence and the 5′ LTR is typically measured, for example, as the number of nucleotides between the 5′ or 3′ nucleotide of the sequence and the 3′ nucleotide of the 5′ LTR. In some embodiments, the polynucleotide can further include a riboswitch in reverse orientation as disclosed elsewhere herein. In some embodiments, the number of nucleotides between the 5′ end of the one or more first transcriptional units and the 5′ end of the one or more second transcriptional units is less than the number of nucleotides between the 3′ end of the one or more first transcriptional units and the 3′ end of the one or more second transcriptional units.

The expression of non-secreted and constitutively active lymphoproliferative elements only by CAR-T cells with active CAR signaling, as in self-driving CARs, can limit the expansion of CAR-T cells in the absence of antigen-binding. Furthermore, after the successful treatment of a tumor, self-driving CAR-T cells proliferate less in the absence of the antigen.

In illustrative embodiments of self-driving CAR embodiments, the inducible promoter is an NFAT-responsive promoter. In some embodiments, the inducible or activatable promoter can be an NFAT-responsive promoter and include one or more NFAT-binding sites. In some embodiments, the one or more NFAT-binding sites can be derived from promoters known in the art to be NFAT-responsive promoters. In some embodiments, the one or more NFAT-binding sites can be derived from an IL-2, IL-4, and/or IL-8 promoters. In illustrative embodiments, the one or more NFAT-binding sites can be derived from an IL-2 promoter. In some embodiments, the NFAT-responsive promoter can include 2, 3, 4, 5, 6, 7, 8, 9, 10, 11, or 12 NFAT-binding sites. In illustrative embodiments, the NFAT-responsive promoter can include 4, 6, or 9 NFAT-binding sites. In some embodiments, the NFAT-binding sites of an NFAT-responsive promoter can include functional sequence variants which retain the ability to bind NFAT, to avoid exact repeats. In some embodiments, the NFAT-responsive promoter is responsive to NFATc1, NFATc2, NFATc3, NFATc4, and/or NFATc5. In some embodiments, the NFAT-responsive promoter includes one or more NFAT-binding sites of SEQ ID NO:352. In some embodiments, the spacing between copies of the NFAT-binding sites can be between 3 and 60 nucleotides or between 6 and 20 nucleotides. In illustrative embodiments the NFAT-responsive promoter comprises 6 NFAT-binding sites and the nucleotide sequence comprises or consists of SEQ ID NO:353 or a functional portion or functional variant thereof.

In some embodiments, a transcriptional unit encoding a lymphoproliferative element includes a minimal constitutive promoter with upstream NFAT-binding sites to generate an inducible or activatable promoter with a low level of transcription even in the absence of an inducing signal. In some embodiments, in the absence of an inducing signal the low level of transcription of a lymphoproliferative element from such an inducible promoter can be less than ½, ¼, ⅕ 1/10, 1/25, 1/50, 1/100, 1/200, 2/250, 1/500, or 1/1,000 the level of transcription of a CAR from the constitutive promoter. In some embodiments, the minimal constitutive promoter can include the minimal IL-2, the minimal CMV, or minimal MHC promoters. In illustrative embodiments, the minimal promoter can be the minimal IL-2 promoter (SEQ ID NO:354) or a functional portion or functional variant thereof. In illustrative embodiments, the NFAT-responsive promoter includes six NFAT-binding sites upstream of the minimal IL-2 promoter and the nucleotide sequence includes or consists of SEQ ID NO:355, or a functional portion or functional variant thereof.

The inducible and constitutive promoters in the polynucleotides disclosed above with a first sequence in reverse orientation and a second sequence in forward orientation, can interfere with each other in unpredictable ways, especially in the presence of a strong constitutive promoter such as the EF1-a, CMV, and CAG promoters. Promoter interference can result in an increase or decrease in transcription from one or both promoters. Promoter interference can also result in a decrease in the dynamic range of an inducible promoter. In some embodiments, an insulator is located between the divergent transcriptional units. In some embodiments, an insulator is located between the inducible and constitutive promoters. In some embodiments, the insulator can be chicken HS4 insulator, Kaiso insulator, SAR/MAR elements, chimeric chicken insulator-SAR elements, CTCF insulator, the gypsy insulator, or the β-globin insulator or fragments thereof known in the art. In some embodiments, the insulator can be b-globin polyA spacer B (SEQ ID NO:356), b-globin polyA spacer A (SEQ ID NO:357), 250 cHS4 insulator v1 (SEQ ID NO:358), 250 cHS4 insulator v2 (SEQ ID NO:359), 650 cHS4 insulator (SEQ ID NO:360), 400 cHS4 insulator (SEQ ID NO:361), 650 cHS4 insulator and b-globin polyA spacer B (SEQ ID NO:362), or b-globin polyA spacer B and 650 cHS4 insulator (SEQ ID NO:3). In some embodiments, the insulator can be in the forward orientation. In other embodiments, the insulator can be in the reverse orientation. A skilled artisan will understand how to incorporate an insulator between promoters to prevent or reduce promoter interference.

In some embodiments, the polynucleotide can include a number of adenosine nucleotides, known as a polyadenylation sequence, following the 3′ end of the sequence encoding a lymphoproliferative element in the reverse orientation. In some embodiments, the polyadenylation sequence can be used with an insulator. In other embodiments, the polyadenylation sequence can be used in the absence of an insulator. In some embodiments, the polyadenylation sequence can be derived from the β-globin polyadenylation sequence or the hGH polyadenylation sequence. In some embodiments, the polyadenylation sequence can be synthetic. In some embodiments, the polyadenylation sequence can include one or more of the sequences selected from hGH polyA (SEQ ID NO:316), SPA1 (SEQ ID NO:317), or SPA2 (SEQ ID NO:318). In some embodiments, the polynucleotide does not include exogenous splice sites. In illustrative embodiments, the polynucleotide does not include exogenous splice sites in the forward or reverse orientation.

In any of the composition and method embodiments for transducing lymphocytes with a self-driving CAR, the polynucleotide can include one or more inhibitory RNA molecules, such as for example, a miRNA or shRNA, as disclosed elsewhere herein. In some embodiments, the inhibitory RNA molecules can be encoded within introns, including for example, an EF1-a intron. In illustrative embodiments, the inhibitory RNA molecules can target any of the targets identified herein, including, but not limited to the Inhibitory RNA Molecules section herein.

In any of the composition and method embodiments for transducing lymphocytes with a self-driving CAR, the inducible promoter can drive expression of a lymphoproliferative element, as disclosed elsewhere herein. In illustrative embodiments, the lymphoproliferative element is a non-secreted and constitutively active lymphoproliferative element.

### Cell Formulations and Methods of Administration

In some embodiments such as those embodiments in which the samples do not undergo a PBMC isolation or granulocyte depletion procedure, at least 5%, at least 10%, at least 20%, at least 25%, at least 30%, at least 40%, at least 50%, or at least 75% of the neutrophils, basophils, and/or eosinophils present in a blood sample that is subjected to a method for modifying herein, are present in the cell formulation, including at the time of the optional delivery (i.e., administering) step. In some embodiments such as those embodiments in which the samples do not undergo a B cell depletion procedure, at least 5%, at least 10%, at least 20%, at least 25%, at least 30%, at least 40%, at least 50%, or at least 75% of the B cells present in a blood sample that is subjected to a method for modifying herein, are present in the cell formulation, including at the time of the optional delivery step. In some embodiments such as those embodiments in which the samples do not undergo a monocyte depletion procedure, at least 5%, at least 10%, at least 20%, at least 25%, at least 30%, at least 40%, at least 50%, or at least 75% of the monocytes present in a blood sample that is subjected to a method for modifying herein, are present in the cell formulation, including at the time of the optional delivery step.

In some embodiments, and in illustrative embodiments in which the cell formulation is administered subcutaneously or intramuscularly, the volume of the cell formulation including the modified lymphocytes is less than traditional CAR-T methods, which typically are infusion-delivery methods, and can be less than, or less than about 1 ml, about 2 ml, about 3 ml, about 4 ml, about 5 ml, about 10 ml, about 15 ml, about 20 ml, or about 25 ml.

The advantageously short time between drawing (collecting) blood and reintroducing the modified lymphocytes into the subject means that in some embodiments, some lymphocytes are associated with the recombinant nucleic acid vectors, in illustrative embodiments the replication incompetent recombinant retroviral particles, and not yet genetically modified. In some embodiments, at least 5% of the modified lymphocytes are not genetically modified. In some embodiments, the modified lymphocytes are genetically modified and contain the polynucleotide, either extrachromosomal or integrated into the genome. In some embodiments, the polynucleotide can be extrachromosomal in at least 5% of the modified lymphocytes. In some embodiments, at least 5% of the modified lymphocytes are not transduced.

The short contacting time in certain embodiments also results in many of the modified lymphocytes in cell formulations herein, having on their surfaces, binding polypeptides, fusogenic polypeptides, and in some embodiments T cell activation elements that originated on the surface of retroviral particles, either through association with the recombinant retroviral particles or by fusion of the retroviral envelopes with the plasma membranes, including at the time of the optional delivery step. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the cell formulation include a pseudotyping element and/or a T cell activation element, e.g., a T cell activating antibody. In some embodiments, the pseudotyping element and/or T cell activation element can be bound to the surface of the modified lymphocytes through, for example, a T cell receptor, CD28, OX40, 4-1BB, ICOS, CD9, CD53, CD63, CD81, CD82, and/or the pseudotyping element and/or T cell activation element can be present in the plasma membrane of the modified lymphocytes.

Cell formulations are provided herein, that included for example T cells and/or NK cells. Such formulations, in illustrative embodiments are provided by methods provided herein. Any of the cell formulations provided herein can include self-driving CAR-T cells. In one aspect, provided herein is a cell formulation comprising a population of self-driving CAR-T cells, such as modified, genetically modified, transcribed, transfected, and/or stably integrated self-driving CAR-T cells in a delivery solution.

Due to the advantageously short time lymphocytes are contacted with recombinant nucleic acid vectors and modified lymphocytes are ex vivo after such contacting in some illustrative embodiments provided herein, in these embodiments some or all of the T and NK cells do not yet express the recombinant nucleic acid or have not yet integrated the recombinant nucleic acid into the genome of the cell, and some of the retroviral particles in embodiments including these, may be associated with, but may have not fused with the target cell membrane, before being used or included in any of the methods or compositions provided herein, including, but not limited to, being introduced or reintroduced back into a subject, or before being used to prepare a cell formulation. Thus, various cell formulation aspects and embodiments are provided herein that can be produced, for example, from these illustrative methods provided herein, such as for example, rapid point of care methods that in illustrative embodiments involve subcutaneous administration. Such cell formulations, including but not limited to those set out immediately below and in the Exemplary Embodiments section herein, can exist at the time of collection of cells after they are contacted with a recombinant retroviral vector and optionally rinsed, and can exist up to and including at the time of administration to a subject, in illustrative embodiments subcutaneously.

In some embodiments, provided herein are cell formulations comprising T cells and/or NK cells, wherein less than 90%, 80%, 75%, 70%, 60%, 50%, 40%, 30%, 25%, 20%, 10%, or 5% of the cells in the cell formulation are T cells and/or NK cells. In some embodiments, cell formulations comprising lymphocytes, NK cells, and/or T cells, are provided wherein at least 4%, 5%, 10%, 15%, 20%, 25%, 30%, 35%, 40%, 45%, 50%, 55%, 60%, 65%, 70%, 75%, 80%, 85%, 90%, or 95% of the lymphocytes, NK cells, and/or in illustrative embodiments T cells in the cell formulation are modified cells. In some embodiments, between 4%, 5%, 10%, 15%, 20%, 25%, 30%, 35%, 40%, 45%, 50%, 55%, 60%, 65%, and 70% of the lymphocytes are modified on the low end of the range and 10%, 15%, 20%, 25%, 30%, 35%, 40%, 45%, 50%, 55%, 60%, 65%, 70%, 75%, 80%, 85%, 90%, and 95% of the lymphocytes are modified cells on the high end of the range, for example between 5% and 95%, 10% and 90%, 25% and 75%, and 25% and 95%. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified lymphocytes within the cell formulation are not genetically modified, transduced, or stably transfected. In some embodiments, between 5%, 10%, 15%, 20%, 25%, 30%, 35%, 40%, 45%, 50%, 55%, 60%, 65%, and 70% of the modified lymphocytes are not genetically modified, transduced, or stably transfected on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified lymphocytes are not genetically modified, transduced, or stably transfected on the high end of the range, for example between 5% and 95%, 10% and 90%, 25% and 75%, and 25% and 95%. In some embodiments, the polynucleotide of genetically modified lymphocytes can be either extrachromosomal or integrated into the genome in these cell formulations that are formed after contacting and incubation, and at the time of optional administration. In some embodiments of these cell formulations, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the genetically modified lymphocytes have an extrachromosomal polynucleotide. In some embodiments, between 5%, 10%, 15%, 20%, 25%, 30%, 35%, 40%, 45%, 50%, 55%, 60%, 65%, and 70% of the modified or genetically modified lymphocytes have an extrachromosomal polynucleotide on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes have an extrachromosomal polynucleotide on the high end of the range, for example between 5% and 95%, 10% and 90%, 25% and 75%, and 25% and 95%. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified or genetically modified lymphocytes are not transduced or stably transfected in these cell formulations, for example as a result of methods for genetically modifying T cells and/or NK cells provided herein. In some embodiments, between 5%, 10%, 15%, 20%, 25%, 30%, 35%, 40%, 45%, 50%, 55%, 60%, 65%, and 70% of the modified or genetically modified lymphocytes are not transduced on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes are not transduced or stably transfected on the high end of the range, for example between 5% and 95%, 10% and 90%, 25% and 75%, and 25% and 95%.

In certain embodiments disclosed herein including subcutaneous delivery of a solution, and cell formulations that are adapted for subcutaneous delivery, fewer of the modified or genetically modified lymphocytes can engraft if delivered intravenously compared to when delivered subcutaneously. In some embodiments, at least 5%, 10%, 15%, 20%, 25%, 30%, 35%, 40%, 45%, 50%, 55%, 60%, 65%, 70%, 75%, 80%, 85%, 90%, or 95% fewer lymphocytes engraft when delivered intravenously compared to when delivered subcutaneously.

In some embodiments, cell formulations, including such formulations in existence at the time of collection of cells after they are contacted with a recombinant retroviral vector and optionally rinsed, and existing up to and including the time of administration to a subject, comprise at least two of unmodified lymphocytes, modified lymphocytes, and genetically modified lymphocytes. In some embodiments, such cell formulations comprise more unmodified lymphocytes than modified lymphocytes. In some embodiments of such cell formulations that are produced by methods provided herein, the percent of T cells and NK cells that are modified, genetically modified, transduced, and/or stably transfected is at least 5%, at least 10%, at least 15%, or at least 20%. As illustrated in the Examples herein, in exemplary methods provided herein for transducing lymphocytes in whole blood, between 1% and 20%, or between 5% and 20%, or between 1% and 15%, or between 5% and 15%, or between 7% and 12% or about 10% of lymphocytes, and in some embodiments of T cells and/or NK cells in the whole blood that is added to a reaction mixture or that is used to create a reaction mixture, are genetically modified and/or transduced and present in resultant cell formulations. In some embodiments, the lymphocytes are not contacted with a recombinant nucleic acid vector, such as a replication incompetent recombinant retroviral particle, and are not modified. In certain illustrative embodiments, the lymphocytes are tumor infiltrating lymphocytes. In some embodiments, the lymphocytes are tumor infiltrating lymphocytes before or after the tumor infiltrating lymphocytes are in contact a recombinant nucleic acid vector. In some embodiments, the lymphocytes comprise both tumor infiltrating lymphocytes and T cells and/or NK cells before or after the T cells and/or NK cells contact a recombinant nucleic acid vector.

In some embodiments, provided herein are cell formulations wherein at least 25%, 30%, 40%, 45%, 50%, 55%, 60%, 65%, 70%, 75%, 80%, 85%, 90%, 95%, 96%, 97%, 98%, 99%, or all of the modified T and/or NK cells in the cell formulation do not express a CAR, or a transposase in certain embodiments, and/or do not have a CAR associated with their cell membrane. In other embodiments, provided herein are cell formulations wherein at least 25%, 30%, 40%, 45%, 50%, 55%, 60%, 65%, 70%, 75%, 80%, 85%, 90%, 95%, 96%, 97%, 98%, 99%, or all of the modified T and/or NK cells in a cell formulation contain recombinant viral reverse transcriptase or integrase. Not to be limited by theory, unlike traditional CAR-T cell processing methods where cells are cultured ex-vivo for days or weeks and many cell divisions, in illustrative methods provided herein, where T cells and/or NK cells are contacted with retroviral particles within hours of delivery, some or most of the reverse transcriptase and integrase present within the retroviral particles that moves into a T cell and/or NK cell after it fuses with a retroviral particle, would still be present in the modified T cells and/or NK cells at the time of delivery. In some embodiments, provided herein are cell formulations wherein at least 25%, 30%, 35%, 40%, 45%, 50%, 55%, 60%, 65%, 70%, 75%, 80%, 85%, 90%, 95%, 96%, 97%, 98%, 99%, or all of the modified T and NK cells in a cell formulation do not express the recombinant mRNA (e.g., encoding a CAR and/or a recombinant transposase). In some embodiments, provided herein are cell formulations wherein at least 20%, 25%, 30%, 35%, 40%, 45%, 50%, 55%, 60%, 65%, 70%, 75%, 80%, 85%, 90%, 95%, 96%, 97%, 98%, 99%, or all of the modified T and NK cells in such cell formulation do not have the recombinant nucleic acid stably integrated into their genomes. In some embodiments, greater than 50%, 60%, 70%, 75%, 80% or 90% of the cells, NK cells, and/or T cells in a cell formulation are viable.

In further embodiments, cell formulations comprising modified lymphocytes that can be introduced or reintroduced in methods herein, include monocytes and/or B cells. In some embodiments, some of the B cells are modified during a contacting step when they are contacted by recombinant nucleic acid vectors, for example, naked DNA vectors, or in illustrative embodiments replication incompetent recombinant retroviral particles. In some embodiments, at least some but not more than 5%, 10%, 15%, 20%, 25%, 30%, 35%, 40%, 45%, 50%, 55%, 60%, 65%, 70%, 75%, 80%, 85%, 90%, or 95% of the B cells are modified in cell formulations, which can optionally be administered or readministered. In illustrative embodiments, some of the B cells are not modified in such formulations and methods. In further illustrative embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the B cells are not modified in such formulations and methods. Thus, in some embodiments, modified lymphocytes are present in cell formulations along with unmodified lymphocytes, which optionally are delivered to a subject intramuscularly or subcutaneously. In some embodiments, the modified lymphocytes in the cell formulations and optionally introduced into the subject can be allogeneic lymphocytes. In such embodiments, the lymphocytes are from a different person, and the lymphocytes from the subject are not modified. In some embodiments, no blood is collected from the subject to harvest lymphocytes.

Neutrophils, in illustrative embodiments, are present in the cell formulation, as a nonlimiting example a cell formulation for delivering modified T cells and/or NK cells subcutaneously, at a concentration too high for intravenous delivery when considering the safety of a subject into which the cell formulation is administered. Not to be limited by theory, and as discussed herein elsewhere, the injection or delivery of neutrophils intravenously can lead to pulmonary compromise, for example, as a result of transfusion-related acute lung injury (TRALI) and/or acute respiratory distress syndrome (ARDS). For example, this situation can arise when the method for producing the modified lymphocytes does not involve a PBMC enrichment step before the cell formulation comprising the modified lymphocytes is prepared, and before the solution is optionally delivered subcutaneously to a subject. Thus, in some embodiments, neutrophils are present in the cell formulation, for example at the time of the optional delivery step. More specifically, in some embodiments, at least 5%, at least 10%, at least 20%, at least 25%, at least 30%, at least 40%, at least 50%, or at least 75% of the neutrophils present in a blood sample that is subjected to a method for modifying herein, are present in the cell formulation, including at the time of the optional delivery step. In some embodiments, at least 5%, at least 10%, at least 20%, at least 25%, at least 30%, at least 40%, at least 50%, at least 60%, at least 70%, or at least 75% of the cells present in the cell formulation are neutrophils, including at the time of the optional delivery step. In some embodiments, between 5%, 10%, 15%, 20%, 25%, 30%, or 40% of the cells present in the cell formulation are neutrophils at the low end of the range and 30%, 40%, 50%, 60%, 70%, or 75% of the cells present in the cell formulation are neutrophils at the high end of the range, including at the time of the optional delivery step, for example between 5% and 50%, 20% and 50%, 30% and 75%, or 50% and 75% of the cells present in the cell formulation are neutrophils, including at the time of the optional delivery step.

In some embodiments, at least 5%, at least 10%, at least 20%, at least 25%, at least 30%, at least 40%, at least 50%, or at least 75% of the monocytes present in a blood sample that is subjected to a method for modifying herein, are present in a cell formulation, including at the time of the optional delivery step. In some embodiments, at least 5%, at least 10%, at least 20%, at least 25%, at least 30%, at least 40%, at least 50%, or at least 75% of the B cells present in a blood sample that is subjected to a method for modifying herein, are present in the resulting cell formulation, including at the time of the optional delivery step. In some embodiments, the cell formulation can include a PBMC fraction, which includes the modified T and NK cells. In some embodiments, at least 1%, 2%, 3%, 4%, 5%, 10%, 15%, 20%, 25%, 50%, 75%, 80%, 85%, 90%, or 95%, or between 1% and 95%, 5% and 95%, 5% and 50%, or 10% and 50% of the modified T and NK cells in a cell formulation are genetically modified.

The volume of cell formulation or other solution administered varies depending on the route of administration, as provided elsewhere herein. Cell formulations injected subcutaneously or intramuscularly typically have smaller volumes than those delivered via infusion. In some embodiments, the volume of the cell formulation or other solution including a suspension of the modified, and in illustrative embodiments genetically modified lymphocytes is not more than 1 ml, 2 ml, 3 ml, 4 ml, 5 ml, 10 ml, 15 ml, 20 ml, 25 ml, 30 ml, 35 ml, 40 ml, 45 ml, or 50 ml. In some embodiments, the volume of the cell formulation or other solution including a suspension of the modified lymphocytes can be between 0.20 ml, 0.25 ml, 0.5 ml, 1 ml, 2 ml, 3 ml, 4 ml, 5 ml, 10 ml, 15 ml, 20 ml, or 25 ml on the low end of the range and 0.5 ml, 1 ml, 2 ml, 3 ml, 4 ml, 5 ml, 10 ml, 15 ml, 20 ml, 25 ml, 30 ml, 35 ml, 40 ml, 45 ml, or 50 ml., 30 ml, 35 ml, 40 ml, 45 ml, 50 ml, 75 ml, 100 ml, 125 ml, 250 ml, 500 ml, or 1000 ml on the high end of the range. Thus, as non-limiting examples, the volume can be between 0.2 ml and 10 ml, 0.5 ml and 10 ml, 0.5 and 2 ml, 1 ml and 250 ml, 1 ml and 100 ml, 10 ml and 100 ml, or 1 ml and 10 ml. In certain illustrative embodiments, less than 10 ml, between 1 ml and 25 ml, and in illustrative embodiments between 1 ml and 3 ml, between 1 ml and 5 ml, or between 1 ml and 10 ml of a cell formulation that includes modified lymphocytes in delivery solution are administered subcutaneously or intramuscularly. In illustrative embodiments, the volume of the solution including the modified lymphocytes can be between 0.20 ml, 0.25 ml, 0.5 ml, 1 ml, 2 ml, 3 ml, 4 ml, and 5 ml on the low end of the range and 0.5 ml, 1 ml, 2 ml, 3 ml, 4 ml, 5 ml, 10 ml, 15 ml, 20 ml, 25 ml, 30 ml, 35 ml, 40 ml, 45 ml, and 50 ml on the high end of the range. In an exemplary embodiment, a 70 kg subject is dosed at 1.0×10⁶ T cells/kg by administering 1 ml of a delivery formulation of T cells at 7.0×10⁷ cells/ml subcutaneously. In some embodiments, the solution can include hyaluronidase when the volume of the solution is at least 2 ml, 3 ml, 4 ml, 5 ml, 10 ml, 15 ml, 20 ml, or 25 ml. In embodiments herein wherein lymphocytes are filtered especially after they are modified, and/or especially where transduction is performed on top of a filter, the delivery solution can be used to resuspend and/or elute cells from the filter in volumes that can be those provided above. As such, in some embodiments, a delivery solution provided herein is an elution solution.

In some embodiments, modified and in illustrative embodiments genetically modified lymphocytes are introduced or reintroduced into the subject by intradermal, intratumoral or intramuscular administration and in illustrative embodiments, subcutaneous administration using a cell formulation present in a subcutaneous delivery device, such as a sterile syringe that is adapted to deliver a solution subcutaneously. In some embodiments, a subcutaneous delivery device is used that holds a solution (e.g., a cell formulation herein) and has an open or openable end, which in illustrative embodiments is the open end of a needle, for administrating the solution (e.g., cell formulation) subcutaneously from the liquid holding portion of the device. Such subcutaneous delivery device is effective for, and in illustrative embodiments adapted for subcutaneous delivery, or effective to inject subcutaneously or adapted to inject subcutaneously. Non-limiting examples of subcutaneous delivery devices that are adapted to deliver a solution subcutaneously include subcutaneous catheters, such as indwelling subcutaneous catheters, such as for example, the Insuflon® (Becton Dickinson) and needless closed indwelling subcutaneous catheter systems, for example with wings, such as for example, the Saf-T-Intima® (Becton Dickinson). In some embodiments, the delivery device can include a pump, for example an infusion pump or a peristaltic pump. In some embodiments, the cell formulation is fluidly connected to any of the needles disclosed herein, for example a needle compatible with, effective for, adapted for, or adapted to deliver subcutaneously or effective to deliver subcutaneously. In illustrative embodiments, the needle can have a gauge between 26 and 30. In some embodiments, the subcutaneous delivery device is a subcutaneous delivery pen. Such pen can include a syringe effective to deliver subcutaneously or adapted to deliver subcutaneously enclosed within a housing and can include a needle guard. Examples of such pens include pens used to deliver sumatriptan. In some embodiments, said cell formulation is present in a subcutaneous delivery device, for example a syringe, with a needle that has penetrated the skin of a subject whose modified T cells and/or NK cells are the modified cells present in the syringe (i.e., the subject receiving the subcutaneous injection is the source of the autologous cells being injected), and in some embodiments is located with its open end in the subcutaneous tissue of the subject. In illustrative embodiments, the subcutaneous delivery device (e.g., syringe) can include a needle that is suitable for subcutaneous administration. Subcutaneous administration typically uses needles with smaller diameters than used with intravenous catheters for blood infusion, which for example can employ a 16 gauge needle. A delivery device such as a syringe that is compatible with intramuscular and, in illustrative embodiments, subcutaneous delivery, is any delivery device (e.g. syringe) that can be successfully used for intramuscular or subcutaneous delivery, and includes those delivery devices (e.g. syringes) that are effective for and adapted for intramuscular or subcutaneous delivery, plus general purpose syringes and syringes that are specifically designed for other purposes that can be successfully employed for intramuscular or subcutaneous delivery in at least some embodiments. As is known, for subcutaneous injection, in illustrative embodiments using a syringe, a needle is inserted through the skin at a 45° to 90° angle. Thus, some embodiments include injecting a cell formulation subcutaneously at an angle of 45° to 90° with respect to the skin, as well as a cell formulation contained within a syringe or other subcutaneous delivery device, having a needle at a 45° to 90° angle to the skin of a subject. A syringe that is effective for intramuscular and, in illustrative embodiments, subcutaneous delivery, or effective to inject intramuscularly or subcutaneously, is a syringe with parameters that are typically effective for intramuscular or subcutaneous delivery, for example, a needle with a gauge between 20 and 22 and a length between 1 inch and 1.5 inches is typically effective for intramuscular delivery and a needle with a gauge between 26 and 30 and a length between 0.5 inches and 0.625 inches is typically effective for subcutaneous delivery. A syringe that is adapted for subcutaneous delivery, or adapted to inject subcutaneously, is any syringe that is specifically made for subcutaneous delivery. One such syringe adapted for subcutaneous delivery uses a core annular flow that allows subcutaneous delivery of highly concentrated biological drug formulations not normally deliverable subcutaneously (Jayaprakash V et al. Adv Healthc Mater. 2020 Aug. 24; e2001022). Another syringe adapted for subcutaneous delivery uses a shorter needle than generally used (Pager A, Expert Opin Drug Deliv. 2020 Aug. 9;1-14). Another syringe adapted for subcutaneous delivery uses a 29G/5-bevel needle with a Thermo Plastic Elastomer (TPE) needle shield (Jaber A et al. BMC Neurol. 2008 Oct. 10; 8:38). In illustrative embodiments, the outer diameter of the needle is less than 0.026″. In some embodiments, the outer diameter of the needle is at most 0.01625″, 0.01865″, 0.01825″, 0.02025″, 0.02255″, or 0.02525″. In some embodiments, the needle is a 17, 18, 19, 20, 21, 22, 23, 24, 25, 26, 26s, 27, 28, 29, or 30 gauge needle. In some embodiments, the length of the needle is not more than 1 inch or 0.5 inches. In illustrative embodiments, the needle is 26, 26s, 27, 28, 29, or 30 gauge needle and the length of the needle is between 0.5 inches and 0/625 inches. In some embodiments, the needle can be a winged infusion set, also known as a butterfly or scalp vein needle. In some embodiments, the introduction or reintroduction can be performed using a subcutaneous catheter.

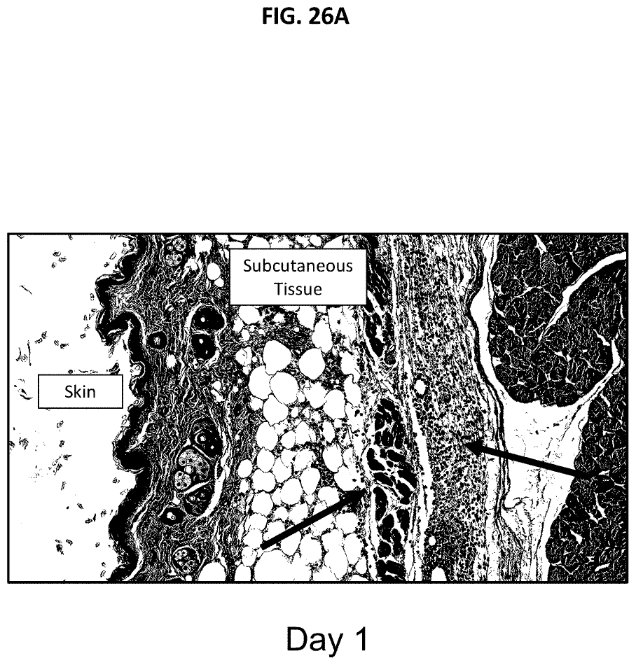

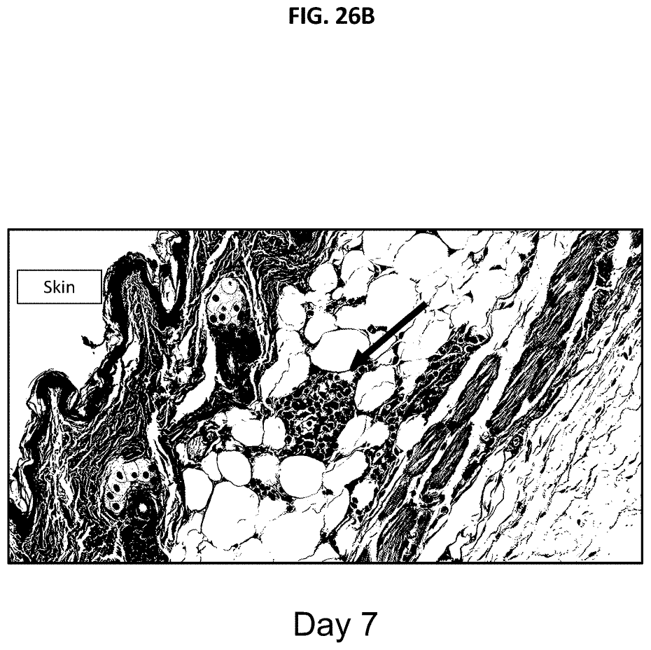

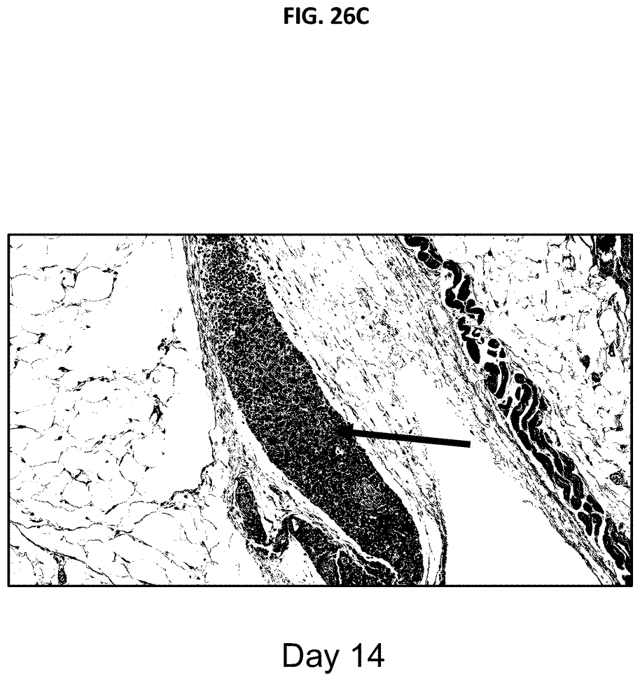

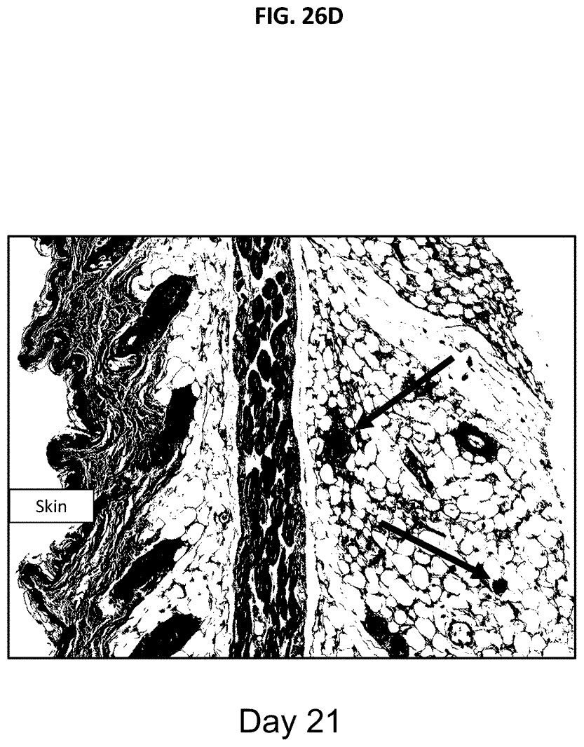

Not to be limited by theory, in contrast to intravenous delivery in which the cells and other components of the cell formulation rapidly disperse, subcutaneous and intramuscular delivery methods provided herein permit the cells and components of the cell formulation to remain in close proximity within a subject, for example in illustrative embodiments for up to several days, several weeks, or even several months as a controlled release while creating a local environment for T cell and/or NK cell activation and expansion maintaining properties similar to what T and NK cells encounter in the lymphoid organs such as the spleen or lymph node. While the absorption of large protein molecules over 20 kDa such as antibodies from subcutaneous sites are absorbed into the blood through the lymphatics over 24 to 72 hours, controlled release of T or NK cells from a local injection site using subcutaneous and intramuscular methods provided herein, were found to involve an initial expansion phase at the site of injection before at least some and typically most of the modified cells migrate through blood vessels and lymphatics to the site of target expression, such as a tumor, and then be detectable through the body. In some embodiments the local injection controlled release will result in genetically modified cells expanding at the site of subcutaneous administration for days (e.g., for up to 5, 7, 14, 17, 21, or 28 days) or months (e.g., for up to 1, 2, 3, 6, 12, or 24 months) with genetically modified CAR-T cells or CAR-NK cells migrating away from the site of subcutaneous administration to other sites of the body, for example to tumors (See e.g., FIG. 26). Thus, genetically modified CAR-T cells can appear in lymphatics or circulation migrating away from a subcutaneous administration site after days (e.g., 1, 2, 3, 4, 5, 6, or 7 days), weeks (e.g., 1, 2, 4, or 4 weeks), and even months (e.g., 1, 2, 3, 6, 12, or 24 months) after modified T cells and/or NK cells are injected subcutaneously into a subject.

This persistence of genetically modified T cells and/or NK cells, such as CAR-T cells, subcutaneously provides an advantageous local environment where other components native or non-native to the subject, such as molecules (ions), macromolecules (e.g., DNA, RNA, peptides, and polypeptides) and/or other cells that can affect the modified CAR-T cells, can be recruited or delivered subcutaneously at or near the site of delivery of the modified CAR-T cells. In fact, tertiary lymphoid structures comprising lymphatic vasculature have been observed after delivery of modified T cells and/or NK cells. Not to be limited by theory, it is believed that such lymphatic vasculature provides a venue for modified T cells and/or NK cells administered subcutaneously to access the local lymphatic circulation, after which they can gain access to the systemic circulation and, for example, access the blood. Such tertiary lymphoid structures have been observed to comprise activated lymphoid cells. Accordingly, provided herein are lymphoid structures comprising aggregates of actively dividing genetically modified T cells and/or NK cells and lymphatic vasculature in proximity to such aggregates. In some embodiments, tertiary lymphoid structures and/or the genetically modified CAR-T cells can persist near a site of subcutaneous administration for at least 1, 2, 3, 4, 5, 6, or 7 days, 1, 2, 3, 4, 5, 6, 7, or 8 weeks, or 1, 2, 3, 4, 5, 6, 7, 8, 9, 10, 11, 12, or 24 months. In illustrative embodiments, tertiary lymphoid structures and/or the genetically modified CAR-T cells persist near the site of subcutaneous administration for at least 1, 2, 3, 4, 5, 6, or 7 days, 1, 2, 3, or 4 weeks, or 1, 2, or 3 months. In some embodiments, tertiary lymphoid structures and/or the genetically modified CAR-T cells can persist near a site of subcutaneous administration for between 1 day and 24 months, 7 days and 12 months, 2 weeks and 6 months, 3 weeks and 8 weeks, or 4 weeks and 6 weeks. In illustrative embodiments, in some embodiments, tertiary lymphoid structures and/or the genetically modified CAR-T cells can persist near a site of subcutaneous administration for between 1 week and 3, 4, 5, 6, 7, 8, 9, or 10 weeks, for example between 1 week and 8 weeks, 1 week and 7 weeks, or 1 week and 6 weeks. In some embodiments, at least 10%, 20%, 30%, 40%, 50%, 60%, 70%, 80%, 90%, or 95% of the cells in tertiary lymphoid structures and/or of the genetically modified cells can remain localized within 1, 2, 3, 4, or 5 cm of site of administration.

Other components that can be delivered along with modified lymphocytes are disclosed in more detail herein, and can be delivered either in the same formulation or in different formulation(s) than the modified T cells and/or NK cells. Furthermore, these other components can be delivered along with the modified T cells and/or NK cells, or can be delivered days (e.g., 1, 2, 3, 4, 5, 6, or 7 days), weeks (e.g., 1, 2, 4, or 4 weeks), or even months (e.g., 1, 2, 3, 6, 12, or 24 months) before or after the modified T cells and/or NK cells. Furthermore, the persistence of genetically modified CAR-T cells near the site of subcutaneous administration further demonstrates an advantage of certain embodiments provided herein wherein the subcutaneous administration is performed near (e.g., within 1, 1, 2, 3, 4, 5, 10, 20, or 30 cm) a site of neoplastic (e.g., cancerous) cells, such as a tumor, or an organ comprising a tumor, including for example, the spleen or lymph nodes in the case of blood cancers.

In some embodiments, the cell formulation is compatible with or even adapted for subcutaneous or intramuscular delivery to keep the cells aggregated locally to enable a controlled release of cells into circulation. The concentration of cells in a cell formulation for subcutaneous or intramuscular delivery in some embodiments is higher than that typically delivered intravenously. In some embodiments the concentration of white blood cells in the cell formulation for subcutaneous or intramuscular delivery is greater than, or greater than about 1.5×10⁸ cells/ml, about 5×10⁸ cells/ml, about 1×10⁹ cells/ml to 1.2×10⁹ cells/ml.

In illustrative embodiments, cells, for example mixtures of modified and unmodified lymphocytes discussed herein, are formulated in a delivery solution such that they are capable of, effective for, and adapted for subcutaneous or intramuscular administration. In fact, certain embodiments of commercial container and kit aspects provided herein, are or include a container of sterile subcutaneous and/or intramuscular delivery solution, which in some embodiments is stored refrigerated. Such delivery solutions are capable of, and in illustrative embodiments effective for, and in further illustrative embodiments adapted for, subcutaneous or intramuscular administration, and in illustrative embodiments subcutaneous administration. To accomplish this, such delivery solutions and resulting cell formulations typically have a pH and ionic composition that provides an environment in which cells to be administered can survive until they are administered, for example for at least 1 hour, and typically can survive for at least 4 hours. Such pH is typically between pH 6.5 to 8.0 or 7.0 and 8.0 or 7.2 to 7.6 and can be maintained by a buffer such as a phosphate buffer or bicarbonate present at a concentration effective for maintaining pH in a target range. In some embodiments, for example, when the replication incompetent recombinant retroviral particle has a polynucleotide that encodes an MRB-CAR, the pH can be between pH 6.0 to pH 7.0, for example, pH 6.2 to pH 7.0, or pH 6.4 to pH 7.0, or pH 6.4 to pH 6.8. The ionic composition of such formulations can for example, include a saline composition with salts, for example 0.8 to 1.0 or about 0.9 or 0.9 percent salts such as sodium chloride. In some embodiments, the delivery solution is or includes PBS/In some embodiments of a delivery solution and resulting cell formulation herein, the concentration of Na⁺ is between 110 mM and 204 mM, the concentration of Cl⁻ is between 98 mM and 122 mM, and/or the concentration of K⁺ is between 3 mM and 6 mM. In illustrative embodiments, a delivery solution and cell formulation comprising the same, contains calcium and/or magnesium. The concentration of calcium can, for example, be between 0.5 mM and 2 mM. The concentration of magnesium can, for example, be between 0.5 mM and 2 mM. In some embodiments, the delivery solution is calcium and magnesium free. In some embodiments, the delivery solution can be Ringer's lactate solution, also known as sodium lactate solution and Hartmann's solution. In illustrative embodiments, Ringer's lactate solution can contain about 130-131 mM sodium, 109-111 mM chloride, 28-29 mM lactate, 4-5 mM potassium, and 1-1.5 mM calcium, and is typically made by mixing sodium chloride (NaCl), sodium lactate (CH₃CH(OH)CO₂Na), calcium chloride (CaCl₂), and potassium chloride (KCl). In some embodiments, the delivery solution can be Plasma-Lyte. In illustrative embodiments, Plasma-Lyte can contain about 150 mM sodium, 5 mM potassium, 1.5 mM magnesium, 98 mM chloride, 27 mM acetate, and 23 mM gluconate. In some embodiments, the delivery solution can include dextrose. In some embodiments, the concentration of dextrose can be at least or about 0.5%, 1%, 2%, 3%, 4%, 5%, 6%, 7%, 8%, 9%, or 10%. In illustrative embodiments, the delivery solution be 5% dextrose in 0.9% NaCl. In some embodiments, the delivery solution is 5% dextrose in 0.9% NaCl (D₅NS)

In some embodiments, the delivery solution and cell formulations contain human serum albumin and/or heparin. In some embodiments the delivery solution and cell formulation contains up to 5% HSA. In some embodiments, the delivery solution contains at least or about 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml. In some embodiments, the delivery solution contains between 10 mg/ml and 30 mg/ml, 10 mg/ml and 100 mg/ml, 20 and 80 mg/ml, or 40 mg/ml and 60 mg/ml. In some embodiments the delivery solution is PBS comprising 2% HSA. In some embodiments, the delivery solution is saline comprising HSA at a concentration of 10, 20, 30, 40, 50, 60, 70, or 80 ml/ml. In some embodiments, the delivery solution is DPBS comprising 2% HSA (W/V, i.e., 2 g per 100 ml). In some embodiments, the delivery solution comprises 30-100 U/ml, 40-100 U/ml, 30-60 U/ml, or 60-80 U/ml heparin. In some embodiments, the delivery solution is a saline solution comprising 30-100 U/ml, 40-100 U/ml, 30-60 U/ml, or 60-80 U/ml heparin, with or without 0.5-5%, 1-5%, or 1-2.5% HSA. Discussion herein regarding concentrations of heparin in reaction mixture aspects, apply equally to delivery solution and cellular formulation aspects. In some embodiments, the delivery solution comprises a saline solution at about pH 7.4 further comprising HSA and sodium bicarbonate.

In some embodiments, the delivery solution is or includes a multiple electrolyte solution suitable for injection into a subject. For example, a delivery solution can be or include a sterile, nonpyrogenic isotonic solution in a container, such as a single dose container. Such solution in certain embodiments is suitable or adapted for intravenous administration or intraperitoneal administration as well as subcutaneous and/or intramuscular administration. In some embodiments, a delivery solution can include a multiple analyte solution for injection into a subject where ach 100 mL contains 526 mg of Sodium Chloride, USP (NaCl); 502 mg of Sodium Gluconate (C₆H₁₁NaO₇); 368 mg of Sodium Acetate Trihydrate, USP (C₂H₃NaO₂.3H₂O); 37 mg of Potassium Chloride, USP (KCl); and 30 mg of Magnesium Chloride, USP (MgCl₂6H₂O) with a pH adjusted to 7.4 (6.5 to 8.0). In illustrative embodiments, the delivery solution contains no antimicrobial agents. The pH is adjusted with sodium hydroxide. As one example, the multiple electrolyte injection solution can be PLASMA-LYTE A Injection pH 7.4 available from various commercial suppliers.

In illustrative embodiments, the cell formulation is never frozen. In illustrative embodiments, the cell formulation contains less than, or less than about 7%, about 6%, about 5%, about 4%, about 3%, about 2%, or about 1% DMSO (v/v). In further illustrative embodiments, the cell formulation contains no DMSO.

A uniform single cell suspension is ideal for intravenous delivery but is not required for subcutaneous or intramuscular administration. In some embodiments, the cell formulation for subcutaneous or intramuscular delivery is a depot formulation or emulsion of cells that promotes cell aggregation, and a delivery solution herein used to prepare such a depot cell formulation, includes the accessory components that provide depot properties. In some embodiments, the cells may be aggregated in the formulation, for example before it is administered to a subject, or for example within 1 hour, 45 minutes, 30 minutes, 15 minutes, 10 minutes, 5 minutes, or 1 minute of cells, for example modified lymphocytes as provided herein, being formulated in a delivery solution, for example comprising an aggregating agent to produce the formulation. In some embodiments, at least 10%, 20%, 25%, 50%, 75%, 90%, 95%, or 99% of the cells in a cell formulation provided herein are aggregated. Such aggregation can be determined, for example, using microscopic counting of individual cells versus cells that are associated with at least one other cell, or by counting the number of cells on average, a cell within a formulation is associated with. In some embodiments the cell formulation is designed for controlled or delayed release with tissue expansion to accommodate cell expansion.

In some embodiments, a delivery solution provided herein, for subcutaneous or intramuscular delivery is a depot formulation. A depot (i.e., sustained release) formulation is typically an aqueous or oleaginous suspension or solution.

Accordingly, in some embodiments, the delivery solution or cell formulation includes components that form an artificial extracellular matrix such as a hydrogel. In some embodiments, a depot delivery solution comprises an effective amount of alginate, collagen, and/or dextran to form a depot formulation. One class of polymers that can be used to make gel-forming biomaterials, and can be included in delivery solutions and cell formulations provided herein, is composed of poly(ethylene glycol) (PEG) and its copolymers with aliphatic polyesters, such as poly(lactic acid) (PLA), poly(D,L-lactic-co-glycolic acid) (PLGA), poly(ε-caprolactone) (PCL) and polyphosphazenes. Other polymers that can be used include thermosensitive triblock copolymers based on poly(N-(2-hydroxypropyl methacrylamide lactate) and poly(ethylenglycol) (p(HPMAm-lac)-PEG), capable of spontaneous self-assembling in physiological environments (Vermonden et. al 2006, Langmuir 22: 10180-10184).

In some embodiments, the hydrogel used in a delivery solution or cell formulation herein, contains hyaluronic acid (HA). Such HA can have carboxylic acid groups that can be modified with 1-ethyl-3-(3-dimethyl aminopropyl)-1-carbodiimide hydrochloride to react with amine groups on proteins, peptides, polymers, and linkers, such as those found on modified lymphocytes provided herein, preferentially in the presence of N-hydroxysuccinimide Antibodies, cytokines and peptides can be chemically conjugated to HA using such methods to produce a hydrogel for co-injection as a cell emulsion in some cell formulation embodiments provided herein. Additionally, in some embodiments, HA in delivery solutions and cell formulations is a polymer (e.g., Healon) and/or are crosslinked (e.g., restylane (Abbive/Allergan)), for example lightly crosslinked, through its —OH groups with agents such as glutaraldehyde to reduce the local catabolism of the material following subcutaneous injection. The HA used in delivery solutions and cell formulations herein, can be of variable length and viscosity. The HA used in delivery solutions and cell formulations herein, can further be crosslinked with other glycosaminoglycans such as chondroitin sulfate (e.g., Viscoat) or polymers or surfactants. A skilled artisan will recognize that the porosity of the matrix and degree of crosslinking can be regulated to ensure cells, such as modified lymphocytes herein, are capable of migration through the hydrogels. Accordingly, a matrix, such as a hydrogel matrix, when used in a cell formulation herein, can be configured for, or adapted to permit migration of cells through the matrix. The degree of substitution of the hydrogel and concentration at the time of crosslinking will influence porosity swelling ratio and Youngs Modulus (or stiffness). Initial 1% substitution of HA with tyramine for example at 1 mg/ml when subsequently crosslinked in the presence of peroxide will result in a hydrogel with higher porosity and lower stiffness than 3% substitution and 5 mg/ml solution. Reducing the shear modulus is desirable in some circumstances to reduce shear force during injection and ensure adequate porosity and half life for cells to expand into the matrix subcutaneously over one to two weeks. In some embodiments, the shear modulus is or is about 2.5 kPa, about 3 kPa, about 3.5 kPa, or about 4 kPa.

In some embodiments the delivery solution, a composition in the kit, or the cell formulation includes one or more cytokines such as IL-2, IL-7, IL-15, or IL-21 and/or cytokine receptor agonists, such as an IL-15 agonist. In some embodiments the cytokine does not bind to a cytokine receptor included in the delivery solution, kit, or cell formulation; and/or does not bind to a cytokine receptor that is encoded by a polynucleotide in the delivery solution, cell formulation, or kit. In some embodiments, the cytokines can be modified cytokines that, not to be limited by theory, selectively activate complexes that drive proliferation. In illustrative embodiments, the modified cytokine is a modified IL-2, for example, a fusion protein with a circularly-permuted IL-2 with the extracellular domain of IL-2Rα (see, e.g., Lopes et al, J Immunother Cancer 2020 April; 8(1): e000673). In some embodiments, the cytokines, modified cytokines, or cytokine receptor agonists can also be administered in one or administrations separate from the cell formulation, before, contemporaneous to, or after the administration including the delivery solution or cell formulation. In some embodiments, two or more separate administrations can be in escalating doses. In some embodiments, two or more administrations can be at the same dose. In some embodiments, two or more administrations can include the same or different cytokines, modified cytokines, and or cytokine receptor agonists. In some embodiments, the separate administrations can be a series of 2, 3, 4, 5, 6, 7, 8, 9, 10, 11, 12, 13, 14, 15, 16, 17, 18, 19, 20, or 21 administrations. In some embodiments, the separate administrations occur on consecutive days.

In some embodiments the cell formulation includes antibodies or polypeptides that are capable of binding CD2, CD3, CD28, OX40, 4-1BB, ICOS, CD9, CD53, CD63, CD81, and/or CD82. The EDC-NHS reaction may be used for linking such proteins to HA or through other intermediates described above. In some embodiments these cytokines, antibodies, or polypeptides are crosslinked to components of a hydrogel. The hydrogel may be mixed with the cell suspension using a syringe connector and two syringes prior to injection. In other embodiments, these cytokines, antibodies, or polypeptides are in solution. In some embodiments, the delivery solution or the cell formulation includes RNA that encodes for these cytokines, antibodies, or polypeptides.

The proliferation and survival of genetically modified T cells and/or NK cells expressing a CAR are promoted by signaling through the CAR when it binds its cognate antigen in the proper context. In some embodiments, the antigen can be added to or co-administered with modified and/or genetically modified T cells and/or NK cells. In some embodiments the antigen is a protein, a glycoprotein, a carbohydrate or fragment thereof such as a peptide, glycopeptide, or functional group. In some embodiments, the antigen can be soluble. In some embodiments, the antigen is from a non-human source. In some embodiments, the antigen can be immobilized on a surface of the artificial matrix, such as a hydrogel. In some embodiments the antigen is a nucleic acid such as DNA or RNA. In some embodiments, the nucleic acid encodes a protein or peptide antigen that is an antigen recognized by the CAR. In illustrative embodiments, the antigen can be expressed on the surface of a cell comprising the nucleic acid encoding the protein or peptide antigen, such that the cell is a target cell, referred to as feeder cells herein. In some embodiments, such target cells are present in large numbers in whole blood and are naturally present in the cell formulation without having to be added. For example, B cells are present in whole blood, isolated TNCs, and isolated PBMCs and would naturally be present in the cell formulation and could serve as target cells for T cells and/or NK cells expressing a CAR directed to CD19 or CD22, as non-limiting examples which are both expressed on B cells. In other embodiments, such target cells are not present in whole blood or are not present in large numbers in whole blood and thus are added exogenously, for example, feeder cells. In some embodiments, target cells can be isolated or enriched from the subject, such as from a tumor sample, using methods known in the art. In other embodiments, cells from the subject or from a source other than the subject, including cell lines, are modified to express the appropriate antigen. In some embodiments, the targets cells are treated to reduce their proliferative capacity by for example, radiation or chemotherapeutic agents before they are administered to a subject. In illustrative embodiments, the antigen expressed on the target cell can include all or a portion of the protein that contains the antigen. In further illustrative embodiments, the antigen expressed on the target cell can include all or a portion of the extracellular domain of the protein that includes the antigen. In some embodiments, the antigen is an antibody that recognizes the ASTR of the CAR, such as an anti-idiotype antibody directed to the scFv domain of the CAR. In some embodiments, the antigen expressed on the target cell can be a fusion with a transmembrane domain that anchors it to the cell surface. Any of the transmembrane domains disclosed elsewhere herein can be used. In some embodiments, the antigen expressed on the target cell can be a fusion with a stalk domain Any of the stalk domains disclosed elsewhere herein can be used. In illustrative embodiments, the antigen can be a fusion with a CD8 stalk and transmembrane domain (SEQ ID NO:24).

In illustrative embodiments, cells in a first cell mixture, for example cells obtained from a subject, are modified with a recombinant nucleic acid vector encoding a target antigen, which can be referred to herein as “artificial antigen presenting cells” or “aAPCs”, and cells in a separate second cell mixture from the same subject are modified to express the CAR that binds the antigen. In some embodiments, where the modified cell that was modified with a vector encoding a target antigen is a T cell, the cell can be called a “T-APC” herein. Such modified T-APCs can include, as non-limiting examples, B cells, dendritic cells, and macrophages, and in illustrative embodiments dendritic cells and macrophages such as where a corresponding CAR-T target is a B cell cancer target, and can be generated using methods provided herein where reaction mixtures for modification (e.g., transduction) include a T cell binding polypeptide, such as a polypeptide directed to CD3. In further illustrative embodiments, the cell mixture is whole blood, isolated TNCs, isolated PBMCs. For example, the first cell mixture can be modified with a recombinant nucleic acid vector encoding a fusion protein of the extracellular domain of Her2 and the transmembrane domain of PDGF and the second cell mixture can be modified with a recombinant nucleic acid vector encoding a CAR directed to HER2. The cells can then be formulated into the delivery solution or otherwise administered to the subject at varying CAR effector cell-to-target-cell ratios. In some embodiments, the effector-to-target ratio at the time of formulation or administration is, or is about 10:1, about 9:1, about 8:1, about 7:1, about 6:1, about 5:1, about 4:1, about 3:1, about 2;1, about 1:1, about 1:2, about 1:3, about 1:5, about 1:6, about 1:7, about 1:8, about 1:9, or about 1:10. In illustrative embodiments, target cells are co-administered with the modified T and/or NK cells subcutaneously or intramuscularly.

The proliferation and survival of genetically modified T cells and/or NK cells expressing a CAR can also be promoted by CAR signaling initiated by cross-linking the CARs by interactions other than through the CAR's ASTRs binding to their cognate antigens. In some embodiments, a small molecule or protein can cross-link and activate CARs on the surface of a cell. In illustrative embodiments, an antibody can cross-link and activate CARs on the surface of a cell. In further illustrative embodiments, the antibody recognizes an epitope in the extracellular domain of the CAR, such as in the stalk or spacer domain In some embodiments, the epitope can be an epitope tag such as His5 (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), HA Tag (YPYDVPDYA; SEQ ID NO:73), RYIRS (SEQ ID NO:79), Phe-His-His-Thr (SEQ ID NO:80), or WEAAAREACCRECCARA (SEQ ID NO:81). In illustrative embodiments the epitope is common to an intracellular antigen that is not reactive to an extracellular receptor. In some embodiments, the epitope tag is the HisX6 tag (SEQ ID NO:77). In some embodiments, the CARs can be cross-linked and activated by adding soluble antibodies that bind the epitope tag. In illustrative embodiments, the CARs can be cross-linked and activated by adding cells, also referred to herein as universal feeder cells, expressing antibodies, or antibody mimetics, that bind the epitope tag. In some embodiments, the antibody or antibody mimetic associates with the cell membrane through a GPI anchor. In illustrative embodiments the antibody or antibody mimetic associates with the cell membrane through a transmembrane domain. In further illustrative embodiments, a stalk or spacer separates the antibody or antibody mimetic, from the transmembrane domain In some embodiments, the same universal feeder cells, for example, universal feeder cells expressing an anti-HisX6 scFv attached to a CD8a stalk and transmembrane domain, can be used with cells that express CARs with ASTRs that bind to different antigens but that include the HisX6 epitope tag in their stalk. These universal feeder cells can be used with cells expressing different CARs containing a common epitope tag. With universal feeder cells, provided the CARs contain the epitope tag, there is no need to generate different feeder cells that express the cognate antigen for CARs containing different ASTRs. The epitope tag on the cells expressing a CAR will be crosslinked by the universal feeder cells to engage clustering and proliferation of the CAR. For example, the anti-HisX6 universal feeder cells can be used with cells expressing a CAR that binds to Her2 and includes the HisX6 epitope tag and could also be used with cells expressing a CAR that binds to Axl and includes the HisX6 epitope tag. The combination of the universal feeder cell and the CAR can enable CAR-T propagation before the cells engage their cognate antigen. Additionally, if the ASTR of the CAR is microenvironment restricted, the use of the universal feeder cell binding to antigen may enable expansion outside that restrictive environment.

Provided herein in another aspect, the delivery solution or the cell formulation includes synthetic RNA. In some embodiments the synthetic RNA includes inhibitory RNAs such as siRNAs directed to one or more targets. The targets for these inhibitory RNAs can be any of the targets for siRNAs or miRNAs disclosed elsewhere herein. In some embodiments, the synthetic RNA includes mRNA encoding for one or more proteins or peptides. In some embodiments, the mRNA encodes for one or more CARs. The CARs may be any CAR composition disclosed herein. In some embodiments, the mRNA encodes for one or more cytokines. In some embodiments, mRNA encodes for IL-2 or a functional variant thereof. In some embodiments, the mRNA encodes for IL-7 or a functional variant thereof. In some embodiments, the mRNA encodes for IL-15 or a functional variant thereof. In some embodiments, the mRNA encodes for IL-21 or a functional variant thereof. In some embodiments, the mRNA encodes one or more proteins or polypeptides that bind and activate a CAR. In some embodiments, the mRNA encodes for an antigen recognized by the ASTR of the CAR. In some embodiments, the mRNA encodes for HER2 or an extracellular domain of HER2. In some embodiments, the mRNA encodes for EGFR or an extracellular domain of EGFR. In some embodiments, the mRNA encodes for Axl or an extracellular domain of Axl. In some embodiments, the mRNA encodes for CD19 or an extracellular domain of CD19. In some embodiments, the mRNA encodes for CD22 or an extracellular domain of CD22. In some embodiments, the mRNA encodes for an antibody recognized by the ASTR of the CAR. In some embodiments, the MRNA encoding for an antibody recognized by the ASTR of the CAR is an anti-ideotype antibody directed to the antibody or scFv of the ASTR. In some embodiments, the mRNA encodes for an antibody that binds an epitope tag of the CAR and can cross-link two CARs as described elsewhere herein. In some embodiments, the mRNA encodes for one or more T and/or NK cell co-stimulatory proteins. Such co-stimulatory proteins may comprise one or more ligands or antibodies to a co-stimulatory receptor on T and/or NK cells. In some embodiments the co-stimulatory receptor is CD28. In some embodiments the co-stimulatory receptor is 4-1BB. In some embodiments, the mRNA encodes a protein or polypeptide that is soluble. In some embodiments, the mRNA encodes a protein or polypeptide that is membrane-bound. In some embodiments, the membrane-bound protein or polypeptide is operatively linked to a transmembrane domain In some embodiments, the synthetic RNA includes both inhibitory RNAs such as siRNAs directed to one or more targets and mRNA encoding for one or more proteins or peptides.

A method for generating mRNA for use in the delivery solution or cell formulation may involve in vitro transcription of a template with specially designed primers, followed by PolyA addition, to produce a construct containing 3′ and 5′ untranslated sequence, a 5′ cap and/or IRES, the nucleic acid to be expressed, and a polyA tail, typically 50-200 bases in length. In some embodiments, the synthetic RNA is a naturally occurring, endogenous RNA for the nucleic acid of interest. In some embodiments, the RNA is not the naturally occurring, endogenous RNA for the nucleic acid of interest. In some embodiments, the RNA is modified to change the stability and/or translation efficiency of the RNA. In some embodiments, the 5′ UTR, 3′UTR, Kozak sequence, polyA tail is modified. In some embodiments, the RNA includes a 5′ cap. In some embodiments, the RNA is encapsulated in lipid-based carrier vehicles. One approach for assembling lipid nanocarriers includes directly mixing of a solution of lipids in ethanol with an aqueous solution of the nucleic acid to obtain lipid nanoparticles (LNPs). In some embodiments, the LNPs comprise PEG-conjugated lipid. PEG conjugated lipids prevent the aggregation during particle formation and allow the controlled manufacturing of particles with defined diameters in the range between approximately 50 nm and 150 nm. PEGylation of nanoparticles can have substantial disadvantages concerning safety and activity. The drawbacks associated with the use of PEGylated nanoparticles has stimulated the development of PEG alternatives. In some embodiments the LNPs do not comprise PEG. In some embodiments, the LNPs comprise poly(glycerol) (PGs), poly(oxazolines), sugar-based systems, and poly(peptides). In some embodiments, the polypeptides include polysarcosine (pSAR). In some embodiments, the LNPs comprise a dendritic cell targeting moiety. In some embodiments, the dendritic cell targeting moiety comprises mannose.

In some embodiments, the RNA can be added to a cell formulation comprising, or co-administered with, modified and/or genetically modified T cells and/or NK cells in cell formulations and methods provided herein. In some embodiments, the RNA is added to the isolated blood of a subject and processed in parallel with the T cells and/or NK cells. In some embodiments, the RNA can be formulated separately from the modified and/or genetically modified T cells and/or NK cells. The synthetic RNA may be delivered by any means known in the art for therapeutic delivery of RNA. In some embodiments, the RNA is delivered intravenously. In some embodiments, the RNA is delivered intraperitoneally. In some embodiments, the RNA is delivered intramuscularly. In some embodiments, the RNA is delivered intratumorally. In some embodiments, the RNA is delivered intradermally In illustrative embodiments, the RNA is delivered subcutaneously. In some embodiments, the RNA is delivered at the same site as the site of administration of the modified and/or genetically modified T cells and/or NK cells. In some embodiments, the RNA is delivered at a site adjacent to the site of administration of the modified and/or genetically modified T cells and/or NK cells. In some embodiments, the RNA is administered once. In some embodiments, the RNA is administered, 2, 3, 4, 5, 6 or more times.

Provided herein in another aspect, is a cell formulation comprising an aggregate(s) of T cells and/or NK cells, wherein the T cells and/or NK cells in illustrative embodiments are modified with a polynucleotide comprising one or more transcriptional units, wherein each of the transcriptional units is operatively linked to a promoter active in T cells and/or NK cells, and wherein the one or more transcriptional units encode a first polypeptide comprising a chimeric antigen receptor (CAR) in a solution, in illustrative embodiments a delivery solution; and further wherein the aggregate comprises at least 4, 5, 6, or 8 T cells and/or NK cells, wherein the cell aggregate is at least 15 μM in its smallest dimension, and/or wherein the cell aggregate is retained, or capable of being retained, by a coarse filter having a diameter of at least 15 μm, or a coarse filter having a diameter of between 15 μm and 60 μm. Large cell aggregates, approximately greater than >40 μm, are dangerous to inject intravenously as they may, for example, lead to microvascular obstruction (Truter et al. 1981. Intensive Care Med 7, 115-119). In some embodiments, cell aggregates have a diameter less than 40 μm. In some embodiments, at least 70%, 80%, 90%, 95%, 96%, 97%, 98%, or 99% of the cell aggregates in a cell formulation have a diameter less than 40 μm. In illustrative embodiments, at least 70%, 80%, 90%, 95%, 96%, 97%, 98%, or 99% of the cell aggregates in a cell formulation have a diameter less than 40 μm, and the cell formulation is administered intravenously.

### Recombinant Retroviral Particles

Recombinant retroviral particles are disclosed in methods and compositions provided herein, for example, to modify T cells and/or NK cells to make genetically modified and/or transduced T cells and/or NK cells. The recombinant retroviral particles are themselves aspects of the present invention. Typically, the recombinant retroviral particles included in aspects provided herein, are replication incompetent, meaning that a recombinant retroviral particle cannot replicate once it leaves the packaging cell. In fact, unless indicated otherwise herein, retroviral particles are replication incompetent, and if such retroviral particles include nucleic acids in their genome that are not native to the retrovirus, they are “recombinant retroviral particles.” In illustrative embodiments, the recombinant retroviral particles are lentiviral particles.

Provided herein in some aspects are replication incompetent recombinant retroviral particles for use in transducing cells, typically lymphocytes and illustrative embodiments T cells and/or NK cells. The replication incompetent recombinant retroviral particles can include an envelope protein. In some embodiments, the envelope protein can be a pseudotyping element. In some embodiments, the envelope protein can be an activation element. In some embodiments, the replication incompetent recombinant retroviral particles include both a pseudotyping element and an activation element. The replication incompetent recombinant retroviral particles can include any of the pseudotyping elements discussed elsewhere herein. In some embodiments, the replication incompetent recombinant retroviral particles can include any of the activation elements discussed elsewhere herein. In one aspect, provided herein is a replication incompetent recombinant retroviral particle including a polynucleotide including: A. one or more transcriptional units operatively linked to a promoter active in T cells and/or NK cells, wherein the one or more transcriptional units encode an engineered T cell receptor or a chimeric antigen receptor (CAR); and B. a pseudotyping element and a T cell activation element on its surface, wherein the T cell activation element is not encoded by a polynucleotide in the replication incompetent recombinant retroviral particle. In some embodiments, the T cell activation element can be any of the activation elements discussed elsewhere herein. In illustrative embodiments, the T cell activation element can be anti-CD3 scFvFc. In another aspect, provided herein is a replication incompetent recombinant retroviral particle, including a polynucleotide including one or more transcriptional units operatively linked to a promoter active in T cells and/or NK cells, wherein the one or more transcriptional units encode a first polypeptide including an engineered T cell receptor or a chimeric antigen receptor (CAR) and a second polypeptide including a lymphoproliferative element. In some embodiments, the lymphoproliferative element can be a chimeric lymphoproliferative element. In illustrative embodiments, the lymphoproliferative element does not comprise IL-7 tethered to the IL-7 receptor alpha chain or a fragment thereof. In some embodiments the lymphoproliferative element does not comprise IL-15 tethered to the IL-2/IL-15 receptor beta chain. In some embodiments of any of the retroviral particle aspects or embodiments provided herein, or any other aspect that includes a retroviral particle, the engineered T cell receptor, CAR, or other transgene is expressed, displayed, and/or otherwise incorporated in the surface of the replication incompetent retroviral particle at a reduced level that is less than 70%, 60%, 50%, 40%, 30%, 20%, 10%, or 5% of the surface expression compared to when the transgene is expressed from an EF1-a or PGK promoter, and in illustrative embodiments, when the transgene is expressed from an EF1-a or PGK promoter in the absence of additional elements (such as degrons or inhibitory RNAs) to reduce such surface expression. In illustrative embodiments of any of the gene vector (e.g. RIP) aspects provided herein, or any other aspect that includes a gene vector, the gene vector is substantially free of the protein transcript encoded by nucleic acid of the gene vector, and/or the RIPs do not express or comprise a detectable amount of the engineered T cell receptor or CAR on their surface, or express or comprise a reduced amount of the engineered T cell receptor or CAR on their surface.

In some aspects, provided herein is a replication incompetent recombinant retroviral particle, comprising a polynucleotide comprising one or more transcriptional units operatively linked to a promoter active in T cells and/or NK cells, wherein the one or more transcriptional units encode a first polypeptide comprising a chimeric antigen receptor (CAR) and a second polypeptide comprising a chimeric lymphoproliferative element, for example a constitutively active chimeric lymphoproliferative element. In illustrative embodiments, the chimeric lymphoproliferative element does not comprise a cytokine tethered to its cognate receptor or tethered to a fragment of its cognate receptor.

Provided herein in some aspects, is a recombinant retroviral particle that includes (i) a pseudotyping element capable of binding to a T cell and/or NK cell and facilitating membrane fusion of the recombinant retroviral particle thereto; (ii) a polynucleotide having one or more transcriptional units operatively linked to a promoter active in T cells and/or NK cells, wherein the one or more transcriptional units encode a first engineered signaling polypeptide having a chimeric antigen receptor that includes an antigen-specific targeting region, a transmembrane domain, and an intracellular activating domain, and a second engineered signaling polypeptide that includes at least one lymphoproliferative element; wherein expression of the first engineered signaling polypeptide and/or the second engineered signaling polypeptide are regulated by an in vivo control element; and (iii) an activation element on its surface, wherein the activation element is capable of binding to a T cell and/or NK cell and is not encoded by a polynucleotide in the recombinant retroviral particle. In some embodiments, the promoter active in T cells and/or NK cells is not active in the packaging cell line or is only active in the packaging cell line in an inducible manner In any of the embodiments disclosed herein, either of the first and second engineered signaling polypeptides can have a chimeric antigen receptor and the other engineered signaling polypeptide can have at least one lymphoproliferative element.

In some aspects, provided herein are replication incompetent recombinant retroviral particles that include a polynucleotide encoding a self-driving CAR. Details regarding such replication incompetent recombinant retroviral particles, and composition and method aspects including a self-driving CAR, are disclosed in more detail herein, for example in the Self-Driving CAR Methods and Compositions section and in the Exemplary Embodiments section.

Various elements and combinations of elements that are included in replication incompetent, recombinant retroviral particles are provided throughout this disclosure, such as, for example, pseudotyping elements, activation elements, and membrane bound cytokines, as well as nucleic acid sequences that are included in a genome of a replication incompetent, recombinant retroviral particle such as, but not limited to, a nucleic acid encoding a CAR; a nucleic acid encoding a lymphoproliferative element; a nucleic acid encoding a control element, such as a riboswitch; a promoter, especially a promoter that is constitutively active or inducible in a T cell; and a nucleic acid encoding an inhibitory RNA molecule. Furthermore, various aspects provided herein, such as methods of making recombinant retroviral particles, methods for performing adoptive cell therapy, and methods for transducing T cells, produce and/or include replication incompetent, recombinant retroviral particles. Replication incompetent recombinant retroviruses that are produced and/or included in such methods themselves form separate aspects of the present invention as replication incompetent, recombinant retroviral particle compositions, which can be in an isolated form. Such compositions can be in dried down (e.g., lyophilized) form or can be in a suitable solution or medium known in the art for storage and use of retroviral particles.

Accordingly, as a non-limiting example, provided herein in another aspect, is a replication incompetent recombinant retroviral particle having in its genome a polynucleotide having one or more nucleic acid sequences operatively linked to a promoter active in T cells and/or NK cells that in some instances, includes a first nucleic acid sequence that encodes one or more (e.g. two or more) inhibitory RNA molecules directed against one or more RNA targets and a second nucleic acid sequence that encodes a chimeric antigen receptor, or CAR, as described herein. In other embodiments, a third nucleic acid sequence is present that encodes at least one lymphoproliferative element described previously herein that is not an inhibitory RNA molecule. In certain embodiments, the polynucleotide incudes one or more riboswitches as presented herein, operably linked to the first nucleic acid sequence, the second nucleic acid sequence, and/or the third nucleic acid sequence, if present. In such a construct, expression of one or more inhibitory RNAs, the CAR, and/or one or more lymphoproliferative elements that are not inhibitory RNAs is controlled by the riboswitch. In some embodiments, two to 10 inhibitory RNA molecules are encoded by the first nucleic acid sequence. In further embodiments, two to six inhibitory RNA molecules are encoded by the first nucleic acid sequence. In illustrative embodiments, 4 inhibitory RNA molecules are encoded by the first nucleic acid sequence. In some embodiments, the first nucleic acid sequence encodes one or more inhibitory RNA molecules and is located within an intron. In certain embodiments, the intron includes all or a portion of a promoter. The promoter can be a Pol I, Pol II, or Pol III promoter. In some illustrative embodiments, the promoter is a Pol II promoter. In some embodiments, the intron is adjacent to and downstream of the promoter active in a T cell and/or NK cell. In some embodiments, the intron is EF1-α intron A.

Recombinant retroviral particle embodiments herein include those wherein the retroviral particle comprises a genome that includes one or more nucleic acids encoding one or more inhibitory RNA molecules. Various alternative embodiments of such nucleic acids that encode inhibitory RNA molecules that can be included in a genome of a retroviral particle, including combinations of such nucleic acids with other nucleic acids that encode a CAR or a lymphoproliferative element other than an inhibitory RNA molecule, are included for example, in the inhibitory RNA section provided herein, as well as in various other paragraphs that combine these embodiments. Furthermore, various alternatives of such replication incompetent recombinant retroviruses can be identified by exemplary nucleic acids that are disclosed within packaging cell line aspects disclosed herein. A skilled artisan will recognize that disclosure in this section of a recombinant retroviral particle that includes a genome that encodes one or more (e.g., two or more) inhibitory RNA molecules, can be combined with various alternatives for such nucleic acids encoding inhibitory RNA molecules provided in other sections herein. Furthermore, a skilled artisan will recognize that such nucleic acids encoding one or more inhibitory RNA molecules can be combined with various other functional nucleic acid elements provided herein, as for example, disclosed in the section herein that focuses on inhibitory RNA molecules and nucleic acid encoding these molecules. In addition, the various embodiments of specific inhibitory RNA molecules provided herein in other sections can be used in recombinant retroviral particle aspects of the present disclosure.

Necessary elements of recombinant retroviral vectors, such as lentiviral vectors, are known in the art. These elements are included in the packaging cell line section and in details for making replication incompetent, recombinant retroviral particles provided in the Examples section and as illustrated in WO2019/055946. For example, lentiviral particles typically include packaging elements REV, GAG and POL, which can be delivered to packaging cell lines via one or more packaging plasmids, a pseudotyping element, various examples which are provided herein, which can be delivered to a packaging cell line via a pseudotyping plasmid, and a genome, which is produced by a polynucleotide that is delivered to a host cell via a transfer plasmid. This polynucleotide typically includes the viral LTRs and a psi packaging signal. The 5′ LTR can be a chimeric 5′ LTR fused to a heterologous promoter, which includes 5′ LTRs that are not dependent on Tat transactivation. The transfer plasmid can be self-inactivating, for example, by removing a U3 region of the 3′ LTR. In some non-limiting embodiments, Vpu, such as a polypeptide comprising Vpu (sometimes called a “Vpu polypeptide” herein) including but not limited to, Src-FLAG-Vpu, is packaged within the retroviral particle for any composition or method aspect and embodiment provided herein that includes a retroviral particle. In some non-limiting embodiments, Vpx, such as Src-FLAG-Vpx, is packaged within the retroviral particle. Not to be limited by theory, upon transduction of a T cells, Vpx enters the cytosol of the cells and promotes the degradation of SAMHD1, resulting in an increased pool of cytoplasmic dNTPs available for reverse transcription. In some non-limiting embodiments, Vpu and Vpx is packaged within the retroviral particle for any composition or method aspect and embodiment that includes a retroviral particle provided herein.

Retroviral particles (e.g., lentiviral particles) included in various aspects of the present invention are in illustrative embodiments, replication incompetent, especially for safety reasons for embodiments that include introducing cells transduced with such retroviral particles into a subject. When replication incompetent retroviral particles are used to transduce a cell, retroviral particles are not produced from the transduced cell. Modifications to the retroviral genome are known in the art to assure that retroviral particles that include the genome are replication incompetent. However, it will be understood that in some embodiments for any of the aspects provided herein, replication competent recombinant retroviral particles can be used.

A skilled artisan will recognize that the functional elements discussed herein can be delivered to packaging cells and/or to T cells using different types of vectors, such as expression vectors. Illustrative aspects of the invention utilize retroviral vectors, and in some particularly illustrative embodiments lentiviral vectors. Other suitable expression vectors can be used to achieve certain embodiments herein. Such expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90: 10613-10617); SV40; herpes simplex virus; or a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus), for example a gamma retrovirus; or human immunodeficiency virus (see, e.g., Miyoshi et al., PNAS 94:10319 23, 1997; Takahashi et al., J Virol 73:7812 7816, 1999); and the like.

As disclosed herein, replication incompetent recombinant retroviral particles are a common tool for gene delivery (Miller, Nature (1992) 357:455-460). The ability of replication incompetent recombinant retroviral particles to deliver an unrearranged nucleic acid sequence into a broad range of rodent, primate and human somatic cells makes replication incompetent recombinant retroviral particles well suited for transferring genes to a cell. In some embodiments, the replication incompetent recombinant retroviral particles can be derived from the Alpharetrovirus genus, the Betaretrovirus genus, the Gammaretrovirus genus, the Deltaretrovirus genus, the Epsilonretrovirus genus, the Lentivirus genus, or the Spumavirus genus. There are many retroviruses suitable for use in the methods disclosed herein. For example, murine leukemia virus (MLV), human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) can be used. A detailed list of retroviruses may be found in Coffin et al (“Retroviruses” 1997 Cold Spring Harbor Laboratory Press Eds: J M Coffin, S M Hughes, H E Varmus pp 758-763). Details on the genomic structure of some retroviruses may be found in the art. By way of example, details on HIV may be found from the NCBI Genbank (i.e., Genome Accession No. AF033819).

In illustrative embodiments, the replication incompetent recombinant retroviral particles can be derived from the Lentivirus genus. In some embodiments, the replication incompetent recombinant retroviral particles can be derived from HIV, SIV, or FIV. In further illustrative embodiments, the replication incompetent recombinant retroviral particles can be derived from the human immunodeficiency virus (HIV) in the Lentivirus genus. Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. in vivo, HIV can infect terminally differentiated cells that rarely divide, such as lymphocytes and macrophages.

In illustrative embodiments, replication incompetent recombinant retroviral particles provided herein contain Vpx polypeptide.

In some embodiments, replication incompetent recombinant retroviral particles provided herein comprise and/or contain Vpu polypeptide.

In illustrative embodiments, a retroviral particle is a lentiviral particle. Such retroviral particle typically includes a retroviral genome within a capsid which is located within a viral envelope.

In some embodiments, DNA-containing viral particles are utilized instead of recombinant retroviral particles. Such viral particles can be adenoviruses, adeno-associated viruses, herpesviruses, cytomegaloviruses, poxviruses, avipox viruses, influenza viruses, vesicular stomatitis virus (VSV), or Sindbis virus. A skilled artisan will appreciate how to modify the methods disclosed herein for use with different viruses and retroviruses, or retroviral particles. Where viral particles are used that include a DNA genome, a skilled artisan will appreciate that functional units can be included in such genomes to induce integration of all or a portion of the DNA genome of the viral particle into the genome of a T cell transduced with such virus.

In some embodiments, the HIV RREs and the polynucleotide region encoding HIV Rev can be replaced with N-terminal RGG box RNA binding motifs and a polynucleotide region encoding ICP27. In some embodiments, the polynucleotide region encoding HIV Rev can be replaced with one or more polynucleotide regions encoding adenovirus E1B 55-kDa and E4 Orf6.

In certain aspects, replication incompetent recombinant retroviral particles can include nucleic acids encoding a self-driving CAR, as disclosed elsewhere herein. As a non-limiting example, such embodiments are retroviral particles whose genome comprises one or more first transcriptional units operably linked to an inducible promoter inducible in at least one of a T cell or an NK cell, and one or more second transcriptional units operably linked to a constitutive T cell or NK cell promoter, wherein the number of nucleotides between the 5′ end of the one or more first transcriptional units and the 5′ end of the one or more second transcriptional units is less than the number of nucleotides between the 3′ end of the one or more first transcriptional units and the 3′ end of the one or more second transcriptional units,
a. wherein at least one of the one or more first transcriptional units encodes a lymphoproliferative element,
b. and wherein at least one of the one or more second transcriptional units encodes a first chimeric antigen receptor (CAR), wherein the CAR comprises an antigen-specific targeting region (ASTR), a transmembrane domain, and an intracellular activating domain

In some embodiments, the replication incompetent recombinant retroviral particles can further display a T cell activation element.

Not to be limited by theory, T cells contacted and transduced with these replication incompetent recombinant retroviral particles that include nucleic acids encoding a self-driving CAR, can receive an initial boost of transcription from the CAR-stimulated inducible promoters as the T cell activation element can stimulate the inducing signal of the CAR-stimulated inducible promoters. The binding of the T cell activation element can induce the calcium ion influx that results in dephosphorylation of NFAT and its subsequent nuclear translocation and binding to NFAT-responsive promoters. The lymphoproliferative elements transcribed and translated from these CAR-stimulated inducible promoters can give an initial increase in proliferation to these cells. In illustrative embodiments, the T cell activation element can be a membrane-bound anti-CD3 antibody, and can be GPI-linked or otherwise displayed on virus. In some embodiments, the membrane-bound anti-CD3 antibody can be fused to a viral envelope protein, such as MuLV, VSV-G, a Henipavirus-G such as NiV-G, or variants and fragments thereof.

In some embodiments, the isolated replication incompetent retroviral particles are a large-scale batch contained in a large-scale container. Such large-scale batch can have titers, for example of 10⁶-10⁸ TU/ml and a total batch size of between 1×10¹⁰ TU and 1×10¹³ TU, 1×10¹¹ TU and 1×10¹³ TU, 1×10¹² TU and 1×10¹³ TU, 1×10¹⁰ TU and 5×10¹² TU, or 1×10¹¹ TU and 5×10¹² TU. In illustrative embodiments, retroviral particles for any aspect or embodiment provided herein are substantially pure, as discussed in more detail herein.

### Retroviral Genome Size

In the methods and compositions provided herein, the recombinant retroviral genomes, in non-limiting illustrative examples, lentiviral genomes, have a limitation to the number of polynucleotides that can be packaged into the viral particle. In some embodiments provided herein, the polypeptides encoded by the polynucleotide encoding region can be truncations or other deletions that retain a functional activity such that the polynucleotide encoding region is encoded by less nucleotides than the polynucleotide encoding region for the wild-type polypeptide. In some embodiments, the polypeptides encoded by the polynucleotide encoding region can be fusion polypeptides that can be expressed from one promoter. In some embodiments, the fusion polypeptide can have a cleavage signal to generate two or more functional polypeptides from one fusion polypeptide and one promoter. Furthermore, some functions that are not required after initial ex vivo transduction are not included in the retroviral genome, but rather are present on the surface of the replication incompetent recombinant retroviral particles via the packaging cell membrane. These various strategies are used herein to maximize the functional elements that are packaged within the replication incompetent recombinant retroviral particles. In some embodiments, the recombinant retroviral genome to be packaged can be between 1,000, 2,000, 3,000, 4,000, 5,000, 6,000, 7,000, and 8,000 nucleotides on the low end of the range and 2,000, 3,000, 4,000, 5,000, 6,000, 7,000, 8,000, 9,000, 10,000, and 11,000 nucleotides on the high end of the range. The retroviral genome to be packaged includes one or more polynucleotide regions encoding a first and second engineering signaling polypeptide as disclosed in detail herein. In some embodiments, the recombinant retroviral genome to be packaged can be less than 5,000, 6,000, 7,000, 8,000, 9,000, 10,000, or 11,000 nucleotides. Functions discussed elsewhere herein that can be packaged include required retroviral sequences for retroviral assembly and packaging, such as a retroviral rev, gag, and pol coding regions, as well as a 5′ LTR and a 3′ LTR, or an active truncated fragment thereof, a nucleic acid sequence encoding a retroviral cis-acting RNA packaging element, and a cPPT/CTS element. Furthermore, in illustrative embodiments a replication incompetent recombinant retroviral particle herein can include any one or more or all of the following, in some embodiments in reverse orientation with respect to a 5′ to 3′ orientation established by the retroviral 5′ LTR and 3′ LTR (as illustrated in WO2019/055946 as a non-limiting example): one or more polynucleotide regions encoding a first and second engineering signaling polypeptide, at least one of which includes at least one lymphoproliferative element; a second engineered signaling polypeptide that can include a chimeric antigen receptor; an miRNA, a control element, such as a riboswitch, which typically regulates expression of the first and/or the second engineering signaling polypeptide; a safety switch polypeptide, an intron, a promoter that is active in a target cell, such as a T cell, a 2A cleavage signal and/or an IRES.

### Kits and Commercial Products

Provided herein in one aspect is a container, such as a commercial container or package, or a kit comprising the same, comprising retroviral particles according to any of the replication incompetent recombinant retroviral particle aspects and embodiments provided herein. As a non-limiting example, the retroviral particles can comprise in their genome a polynucleotide comprising one or more nucleic acid sequences operatively linked to a promoter active in T cells and/or NK cells. In some embodiments, a nucleic acid sequence of the one or more nucleic acid sequences can encode a lymphoproliferative element and/or a chimeric antigen receptor (CAR) comprising an antigen-specific targeting region (ASTR), a transmembrane domain, and an intracellular activating domain In some embodiments, a nucleic acid sequence of the one or more nucleic acid sequences can encode one, two or more inhibitory RNA molecules directed against one or more RNA targets.

The container that contains the recombinant retroviral particles in any aspect or embodiment, including commercial container as well as kits, can be a tube, vial, well of a plate, or other vessel for storage of retroviral particles. In fact, some aspects provided herein, comprise a container comprising retroviral particles, wherein such retroviral particles include any nucleic acid(s) or other component(s) disclosed herein. Such container in illustrative embodiments includes substantially pure replication incompetent recombinant retroviral particles, sometimes referred to herein for shorthand, as substantially pure retroviral particles. Typically, a preparation and/or container of substantially pure retroviral particles is sterile, and negative for mycoplasma, replication competent retroviruses of the same type, and adventitious viruses according to standard protocols (see e.g., “Viral Vector Characterization: A Look at Analytical Tools”; Oct. 10, 2018 (available at https://cellculturedish.com/viral-vector-characterization-analytical-tools/)). Exemplary methods for generating substantially pure retroviral particles are provided in the Examples herein. For such methods, viral supernatants were purified by a combination of depth filtration, TFF, benzonase treatment, diafiltration, and formulation. In certain illustrative embodiments, substantially pure retroviral particles meet all of the following characteristics based on quality control testing results:
a. negative for mycoplasma;
b. endotoxin at less than 25 EU/ml, and in certain further illustrative embodiments, less than 10 EU/ml;
c. absence of replication competent retroviruses detected of the same type as purposefully in the container (e.g., lentiviruses) detected;
d. absence of adventitious viruses detected;
e. less than 1 pg host cell DNA/viral TU, and in certain further illustrative embodiments, less than 0.3 pg/TU;
f. less than 100 residual plasmid copies/viral TU, and in certain further illustrative embodiments, less than 10 copies/viral TU of any plasmid used to make the recombinant retroviral particles.
g. less than 1 ng HEK protein/TU, and in certain further illustrative embodiments, less than 50 pg HEK protein/TU.
h. Greater than 100 TU/ng P24 protein, and in certain further illustrative embodiments, greater than 10,000 TU/ng P24 protein.

Retroviral particles are typically tested against release specifications that include some or all of those provided above, before they are released to a customer. Potency of each particle may be defined on the basis of p24 viral capsid protein by ELISA, viral RNA genome copies by q-RT PCR, measurement of reverse transcriptase activity by qPCR-based product-enhanced RT (PERT) assay but can all be converted to infectious titer by measuring functional gene transfer Transducing Units (TUs) in a bioassay.

Determination of infectious titer of purified bulk retrovirus material and finished product by bioassay and qPCR is an exemplary analytical test method for the determination of infectious titer of retroviruses. An indicator cell bank (such as F1XT) may be grown for example in serum free media, seeded at 150,000 cells per well, followed by exposure to serial dilutions of the retrovirus product. Dilutions of purified retrovirus particles are made on indicator cells, for example from 1:200 to 1:1,600. A reference standard virus may be added for system suitability. Following 4 days of incubation with retrovirus, the cells are harvested, DNA extracted and purified. A standard curve, for example from 100-10,000,000 copies/well, of human genome and unique retroviral genome sequence plasmid pDNA amplicons are used followed by addition of genomic DNA of the cell samples exposed to retrovirus particles. For each PCR reaction, the Cq values of both the retrovirus amplicon and the endogenous control such as humanRNAseP are extrapolated back to copies per reaction. From these values the integrated genome copy number is calculated. In some cases, indicator cells such as 293T have been characterized as being triploid, hence 3 copies of a single copy gene per cell should be utilized in the calculation. Using the initial viable cell count per well, the volume of retrovirus added to the cells and the genome copy number ratio a Transducing Unit (TU) per ml retrovirus particles may be determined.

Potency testing can include potency testing against release specifications with purity and specific activity. For example, potency release testing of final product can include measurement of the number of Transducing Units (TU) can be compared to viral particle quantity (e.g., by performing an ELISA against a viral protein, for example for lentivirus by performing a p24 capsid protein ELISA with a cutoff of at least 100, 1,000, 2,000 or 2,500 TU/ng p24), and CAR functionality, for example by measuring interferon gamma release by a reporter cell line exposed to gene modified cells.

In any of the kit or isolated replication incompetent recombinant retroviral particle aspects herein, that include a container of such retroviral particles, sufficient recombinant retroviral particles are present in the container to achieve an MOI (the number of Transducing Units, or TUs applied per cell) in a reaction mixture made using the retroviral particles, of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15, or to achieve an MOI of at least 0.1, 0.5, 1, 2, 2.5, 3, 5, 10 or 15. The Transducing Units of virus particles provided in the kit should enable the use an MOI that prevents producing too many integrants in an individual cell, on average less than 3 lentigenome copies per cellular genome and more preferably 1 copy per cell. For kit and isolated retroviral particle embodiments, such MOI can be based on 1, 2.5, 5, 10, 20, 25, 50, 100, 250, 500, or 1,000 ml of reaction mixture assuming 1×10⁶ target cells/ml, for example in the case of whole blood, assuming 1×10⁶ PBMCs/ml of blood. Accordingly, a container of retroviral particles can include between 1×10⁵ and 1×10⁹, 1×10⁵ and 1×10⁸, 1×10⁵ and 5×10⁷, 1×10⁵ and 1×10⁷, 1×10⁵ and 1×10⁶; 5×10⁵ and 1×10⁹; 5×10⁵ and 1×10⁸, 5×10⁵ and 5×10⁷, 5×10⁵ and 1×10⁷, 5×10⁵ and 1×10⁶, or 1×10⁷ and 1×10⁹, 1×10⁷ and 5×10⁷, 1×10⁶ and 1×10⁷, and 1×10⁶ and 5×10⁶ TUs. In certain illustrative embodiments, the container can contain between 1×10⁷ and 1×10⁹, 5×10⁶ and 1×10⁸, 1×10⁶ and 5×10⁷, 1×10⁶ and 5×10⁶ or between 5×10⁷ and 1×10⁸ retroviral Transducing Units. Not to be limited by theory, such numbers of particles would support between 1 and 100 ml of blood at an MOI of between 1 and 10. In some illustrative embodiments, as indicate herein, as little as 10 ml, 5 ml, 3 ml, or even 2.5 ml of blood can be processed for T cell and/or NK cell modification and optionally subcutaneous and/or intramuscular administration methods provided herein. Thus, an advantage of the present methods is that in some illustrative embodiments, they require far fewer retroviral particle Transducing Units than prior methods that involve nucleic acids encoding a CAR, such as CAR-T methods.

Each container that contains retroviral particles, can have, for example, a volume of between 0.05 ml and 5 ml, 0.05 ml and 1 ml, 0.05 ml and 0.5 ml, 0.1 ml and 5 ml, 0.1 ml and 1 ml, 0.1 ml and 0.5 ml, 0.1 and 10 ml, 0.5 and 10 ml, 0.5 ml and 5 ml, 0.5 ml and 1 ml, 1.0 ml and 10.0 ml, 1.0 ml and 5.0 ml, 10 ml and 100 ml, 1 ml and 20 ml, 1 ml and 10 ml, 1 ml and 5 ml, 1 ml and 2 ml, 2 ml and 20 ml, 2 ml and 10 ml, 2 ml and 5 ml 0.25 ml to 10 ml, 0.25 to 5 ml, or 0.25 to 2 ml.

In certain embodiments, retroviral particles in the container are GMP-grade, or cGMP-grade retroviral particles (i.e., produced under GMP or current GMP requirements according to a regulatory agency), or the product of a retroviral manufacturing process performed using GMP systems. Such retroviral particles are typically made using a USA FDA (i.e., U.S. GMP or U.S. cGMP), EMA (i.e., EMA GMP or EMA cGMP), or National Medical Products Administration (NMPA) of China (i.e., Chinese FDA) (i.e., NMPA GMP or NMPA cGMP) good manufacturing practice (GMP), for example using GMP quality systems and GMP procedural controls. These products are typically produced in facilities that meet GMP or cGMP requirements. Such products are typically manufactured under a strict quality management system based on GMP or cGMP regulations. GMP-grade retroviral particles are typically sterile. This can be accomplished for example, by filtering retroviral particles, for example substantially pure retroviral particles, with a 0.45 μm or a 0.22 μm filter. GMP-grade retroviral particles are typically substantially pure, and prepared with control manufacturing test specifications for potency, quality and safety.

In some embodiments, the solution comprising retroviral particles in the container is free of detectable bovine proteins, which can be referred to as “bovine-free”. For example, such solution of retroviral particles can be bovine free because bovine proteins, such as bovine serum proteins, are not used in culturing the packaging cells during retrovirus production. In some embodiment, the solution of retroviral particles is GMP-grade and bovine-free. Substantially pure nucleic acid solutions are typically bovine-free and manufactured in bovine-free broth.

In some aspects, provided herein is a kit for modifying NK cells and/or in illustrative embodiments, T cells. Such a kit in certain embodiments, includes one or a plurality of containers containing polynucleotides, typically substantially pure polynucleotides comprising one or more first transcriptional units operatively linked to a promoter active in T cells and/or NK cells, wherein the one or more first transcriptional units encode a first polypeptide comprising a first chimeric antigen receptor (CAR), sometimes referred to as a first CAR, and one or more containers of accessory component(s), also called accessory kit components herein. The polynucleotides (e.g., retroviral particles) can be stored frozen, for example at −70° C. or lower (e.g., −80° C.).

In illustrative embodiments, the polynucleotides encoding the CAR are located in the genome of retroviral particles, typically substantially pure retroviral particles, according to any of the replication incompetent recombinant retroviral particle aspects and embodiments provided herein. In illustrative embodiments, the replication incompetent recombinant retroviral particles in the kit comprise a polynucleotide comprising one or more transcriptional units operatively linked to a promoter active in T cells and/or NK cells, wherein the one or more first transcriptional units encode a first polypeptide comprising a first chimeric antigen receptor (CAR) and optionally encode a second polypeptide comprising a lymphoproliferative element, according to any of the embodiments provided herein.

The accessory kit components can include one or more of the following:
a. one or more containers containing a delivery solution compatible with, in illustrative embodiments effective for, and in further illustrative embodiments adapted for subcutaneous and/or intramuscular administration as provided herein;
b. one or more containers of hyaluronidase as provided herein;
c. one or more blood bags such as a blood collection bag, in illustrative embodiments comprising an anticoagulant in the bag, or in a separate container, a blood processing buffer bag, a blood processing waste collection bag, and a blood processing cell sample collection bag;
d. one or more sterile syringes compatible with, in illustrative embodiments effective for, and in further illustrative embodiments adapted for, subcutaneous or intramuscular delivery of T cells and/or NK cells;
e. a T cell activation element as disclosed in detail herein, for example anti-CD3 provided in solution in the container containing the retroviral particle, or in a separate container, or in illustrative embodiments, is associated with a surface of the replication incompetent retroviral particle;
f. one or a plurality of leukoreduction filtration assemblies;
g. one or more containers containing a solution or media compatible with, in illustrative embodiments effective for, and in further illustrative embodiments adapted for transduction of T cells and/or NK cells;
h. one or more containers containing a solution or media compatible with, in illustrative embodiments effective for, and/or in further illustrative embodiments adapted for rinsing T cells and/or NK cells;
i. one or more containers containing a pH-modulating pharmacologic agent;
j. one or more containers containing polynucleotides, typically substantially pure polynucleotides (e.g. found within recombinant retroviral particles according to any embodiment herein), comprising one or more second transcriptional units operatively linked to a promoter active in T cells and/or NK cells, wherein the one or more second transcriptional units encode a polypeptide comprising a second CAR directed against a different target epitope, and in certain embodiments a different antigen, in illustrative embodiments found on a same target cancer cell (e.g. B cell);
k. one or more containers containing a cognate antigen for the first CAR and/or the second CAR encoded by the nucleic acids (e.g., retroviral particles); and
l. Instructions, either physically or digitally associated with other kit components, for the use thereof, for example for modifying T cells and/or NK cells, for delivering modified T cells and/or NK cells to a subject subcutaneously or intramuscularly, and/or for treating tumor growth or cancer in a subject.

In some embodiments, the blood bags can hold 5, 10, 15, 20, 25, 50, 75, 100, 150, 200, 250, 300, 400, or 500 ml or less of blood. In some embodiments, the blood bags can hold at least 5, 10, 15, 20, 25, 50, 75, 100, 150, 200, 250, 300, 400, or 500 ml of blood. In some embodiments, the blood bags can hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. In some embodiments, the blood bag can hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. For example, the blood bag can hold between 1 and 10 ml, 5 and 25 ml, 10 and 50 ml, 25 and 100 ml, 50 and 200 ml, or 100 and 500 ml of blood. In some embodiments, the blood bags can include heparin. In other embodiments, the blood bags do not include heparin.

In any of the kit aspects and embodiments herein that include an antigen or a cognate antigen, less than 50%, 40%, 30%, 20%, 10%, 5%, or 1% of the polypeptides in the kit are non-human, i.e., produced from non-human sources.

In some embodiments, the kit may be a single-pack/use kit, but in other embodiments the kit is a multi-pack or multi-use kit for the processing of more than one blood sample from contacting with nucleic acids encoding a CAR optionally thru subcutaneous administration. Typically, a container of nucleic acids encoding a CAR (and optionally a paired container of nucleic acids encoding a second CAR in certain embodiments) in the kit is used for one performance of a method for modifying T cells and/or NK cells and optionally subcutaneous administration. The container(s) containing nucleic acids encoding a CAR and optionally a second CAR is typically stored and shipped frozen. Thus, a kit can include sufficient containers (e.g., vials) of nucleic acids encoding a CAR (and optionally paired containers encoding a second CAR in certain embodiments) for 1, 2, 3, 4, 5, 6, 10, 12, 20, 24, 50 and 100 performances of a method for modifying a T cell and/or NK cell provided herein, and thus can include 1, 2, 3, 4, 5, 6, 10, 12, 20, 24, 50 and 100 containers (e.g., vials) of nucleic acids encoding the CAR (e.g., retroviral particles), and similarly is considered a 1, 2, 3, 4, 5, 6, 10, 12, 20, 24, 50 and 100 pack, performance, administration or X kit, respectively. Similarly, accessory components in the kit would be provided for similar numbers of performances of a method for modifying T cells and/or NK cells and optionally subcutaneous administration, using the kit.

The one or more leukoreduction filtration assemblies, if present in such a kit, typically include(s) one or a plurality of leukoreduction filters or leukoreduction filter sets, each typically within a filter enclosure, as exemplified by the illustrative assembly of FIG. 2, as well as a plurality of connected sterile tubes connected or adapted to be connected thereto, and a plurality of valves connected or adapted to be connected thereto, that are adapted for use in a single-use closed blood processing system. Typically, there is one leukoreduction filtration assembly for each container of nucleic acid encoding a CAR in a kit. Thus a 20-pack kit in illustrative embodiments, includes 20 vials of nucleic acids encoding a CAR and 20 leukoreduction filtration assemblies. In some embodiments, a kit herein comprises one or a plurality of containers containing nucleic acids and one or more leukoreduction filtration assemblies. Such a kit can optionally be intended to be used for administration to a subject via any route including for example, infusion or in illustrative embodiments intramuscular and/or in further illustrative embodiments, subcutaneous delivery. Thus, such a kit optionally includes other accessory components that are intended to be used with such route of administration. The one or more containers of subcutaneous or intramuscular delivery solution is discussed in more detail herein, is typically sterile and can include a total combined volume, or individually per container, of 100 ml to 5 L, 1 ml to 1 L, 1 ml to 500 ml, 1 ml to 250 ml, 1 ml to 200 ml, 1 ml to 100 ml, 1 ml to 10 ml, or 1 ml to 5 ml; 5 ml to 1 L, 5 ml to 500 ml, 5 ml to 250 ml, 5 ml to 100 ml, 5 ml to 10 ml, or approximately 5 ml. In some illustrative embodiments, the kit comprises a plurality of containers of subcutaneous delivery solution, with each container having a volume of between 10 ml and 200 ml, 10 ml and 100 ml, 1 ml and 20 ml, 1 ml and 10 ml, 1 ml and 5 ml, 1 ml and 2 ml, 2 ml and 20 ml, 2 ml and 10 ml, 2 ml and 5 ml, 0.25 ml to 10 ml, 0.25 to 5 ml, or 0.25 to 2 ml. In illustrative embodiments, there is one container of delivery solution for each container of nucleic acid encoding a CAR in a kit. Thus, a 20-pack kit in illustrative embodiments, includes 20 vials of nucleic acids encoding a CAR and 20 containers of sterile delivery solution.

In certain kit aspects, provided herein are embodiments in which either or both the container(s) containing nucleic acids encoding a first CAR and optionally nucleic acids encoding a second CAR, are nucleic acids according to any of the self-driving CAR embodiments provided herein. In such embodiments, accessory components of the kit can further include one or more of the following:
a. one or more containers containing a delivery solution adapted for, compatible with, and/or effective for, intravenous or intraperitoneal administration as provided herein; and
b. Instructions, either physically or digitally associated with other kit components, for the use thereof, for example for delivering modified T cells and/or NK cells to a subject intravenously or intraperitoneally.

In certain aspects, provided herein are the use of a replication incompetent recombinant retroviral particle in the manufacture of a kit for modifying a T cell or NK cell, wherein the use of the kit includes: contacting the T cell or NK cell ex vivo with the replication incompetent recombinant retroviral particle, wherein the replication incompetent recombinant retroviral particle includes a pseudotyping element on a surface and a T cell activation element on the surface, wherein said contacting facilitates transduction of the T cell or NK cell by the replication incompetent recombinant retroviral particle, thereby producing a modified and in illustrative embodiments genetically modified T cell or NK cell.

In some aspects, provided herein are aspects that include the use of a replication incompetent recombinant retroviral particle in the manufacture of a kit for modifying a T cell or NK cell. Details regarding polynucleotides, and replication incompetent recombinant retroviral particles that contain such polynucleotides are disclosed in more detail herein, and in the Exemplary Embodiments section. In some embodiments, the T cell or NK cell can be from a subject. In some embodiments, the T cell activation element can be membrane-bound. In some embodiments, the contacting can be performed for between 1, 2, 3, 4, 5, 6, 7, or 8 hours on the low end of the range and 4, 5, 6, 7, 8, 10, 12, 15, 18, 21, and 24 hours on the high end of the range, for example, between 1 and 12 hours. The replication incompetent recombinant retroviral particle for use in the manufacture of a kit can include any of the aspects, embodiments, or subembodiments discussed elsewhere herein.

Furthermore, provided herein in another aspect is a container, such as a commercial container or package, or a kit comprising the same, comprising isolated packaging cells, in illustrative embodiments isolated packaging cells from a packaging cell line, according to any of the packaging cell and/or packaging cell line aspects provided herein. In some embodiments, the kit includes additional containers that include additional reagents such as buffers or reagents used in methods provided herein. Furthermore, provided herein in certain aspects are use of any replication incompetent recombinant retroviral particle provided herein in any aspect, in the manufacture of a kit for modifying and in illustrative embodiments genetically modifying a T cell or NK cell according to any aspect provided herein. Furthermore, provided herein in certain aspects are use of any packaging cell or packaging cell line provided herein in any aspect, in the manufacture of a kit for producing the replication incompetent recombinant retroviral particles according to any aspect provided herein.

In another aspect, provided herein is a pharmaceutical composition for treating or preventing cancer or tumor growth comprising a replication incompetent recombinant retroviral particle as an active ingredient. In another aspect, provided herein is an infusion composition or other cell formulation for treating or preventing cancer or tumor growth comprising a replication incompetent recombinant retroviral particle. The replication incompetent recombinant retroviral particle of the pharmaceutical composition or infusion composition can include any of the aspects, embodiments, or subembodiments discussed above or elsewhere herein.

### Compositions and Methods for Transducing Lymphocytes in Additional Blood Components

Provided herein in certain aspects, is a method of transducing, genetically modifying, and/or modifying peripheral blood mononuclear cells (PBMCs), or lymphocytes, typically T cells and/or NK cells, and in certain illustrative embodiments resting T cells and/or resting NK cells, in a reaction mixture comprising blood, or a component thereof, and/or an anticoagulant, that includes contacting the lymphocytes with replication incompetent recombinant retroviral particles in the reaction mixture. Such reaction mixture itself represents a separate aspect provided herein. The reaction mixture in illustrative embodiments comprises the lymphocytes and the replication incompetent recombinant retroviral particles, a T cell activation element and one or more additional blood components set out below that in illustrative embodiments are present because the reaction mixture comprises at least 10% whole blood, wherein the replication incompetent recombinant retroviral particles typically comprises a binding polypeptide and a fusogenic polypeptide, and in illustrative embodiments a pseudotyping element on its surface. In such methods, the contacting (and incubation under contacting conditions) facilitates association of the lymphocytes with the replication incompetent recombinant retroviral particles, wherein the recombinant retroviral particles genetically modify and/or transduce the lymphocytes. The reaction mixture of these method or reaction mixture aspects comprises at least 10% unfractionated whole blood (e.g. at least 10%, 20%, 25%, 50%, 60%, 70%, 80%, 90%, 95%, or 99% whole blood) and optionally an effective amount of an anticoagulant; or the reaction mixture further comprises at least one additional blood or blood preparation component that is not a PBMC, for example the reaction mixture comprises an effective amount of an anticoagulant and one or more blood preparation component that is not a PBMC. A percentage of whole blood is the percent by volume of a reaction mixture that was made using unfractionated whole blood. For example, where a reaction mixture is formed by adding replication incompetent recombinant retroviral particles to whole blood, and in illustrative embodiments unfractionated whole blood, the percentage of whole blood in the reaction mixture is the volume of whole blood by the total volume of the reaction mixture times 100. In illustrative embodiments such blood or blood preparation component that is not a PBMC is one or more (e.g., at least one, two, three, four, or five) or all of the following additional components:
a) erythrocytes, wherein the erythrocytes comprise between 1 and 60% of the volume of the reaction mixture;
b) neutrophils, wherein the neutrophils comprise at least 10% of the white blood cells in the reaction mixture, or wherein the reaction mixture comprises at least 10% as many neutrophils as T cells;
c) basophils, wherein the basophils comprise at least 0.05% of the white blood cells in the reaction mixture;
d) eosinophils, wherein the reaction mixture comprises at least 0.1% of the white blood cells in the reaction mixture;
e) plasma, wherein the plasma comprises at least 1% of the volume of the reaction mixture; and
f) an anticoagulant,
(such blood or blood preparation components a-f above referred to herein as (“Noteworthy Non-PBMC Blood or Blood Preparation Components”)).

In any of the aspects disclosed herein that include a percentage of whole blood, the percentage is based on volume. For example, in certain embodiments at least 25% of the volume of a reaction mixture can be whole blood. Thus, in such embodiments at least 25 ml of 100 ml of such reaction mixture, would be whole blood.

The one or more additional blood components that is not a PBMC that is found in certain embodiments herein, are present in certain illustrative embodiments of the reaction mixture (including related use, cell formulation, modified and in illustrative embodiments genetically modified T cell or NK cell, or method for modifying T cells and/or NK cells aspects provided herein) because in these illustrative embodiments the reaction mixture comprises at least 10% whole blood, and in certain illustrative embodiments, at least 25%, 50%, 75%, 90%, or 95% whole blood, or for example between 25% and 95% whole blood. In these illustrative embodiments, such reaction mixtures are formed by combining whole blood with an anticoagulant (for example by collecting whole blood into a blood collection tube comprising an anticoagulant), and adding a solution of recombinant retroviruses to the blood with anticoagulant. Thus, in illustrative embodiments, the reaction mixture comprises an anticoagulant as set out in more detail herein, for example in the Exemplary Embodiments section. In some embodiments, the whole blood is not, or does not comprise, cord blood.

The reaction mixture in illustrative embodiments of these aspects, is formed by some volume of whole blood added directly to other reaction mixture components to form the reaction mixture. Thus, the reaction mixture in such embodiments is formed by a method that typically does not include a PBMC enrichment procedure. Thus, typically such reaction mixtures include additional components listed in a)-f) above, which are not PBMCs. Furthermore, in illustrative embodiments, the reaction mixture comprises all of the additional components listed in a) to e) above, because the reaction mixture comprises substantially whole blood, or whole blood. “Substantially whole blood” is blood that was isolated from an individual(s), has not been subjected to a PBMC enrichment procedure, and is diluted by less than 50% with other solutions. For example, this dilution can be from addition of an anticoagulant as well as addition of a volume of fluid comprising retroviral particles. Further reaction mixture embodiments for methods and compositions that relate to transducing lymphocytes in whole blood, are provided herein.

In yet another aspect provided herein, is use of replication incompetent recombinant retroviral particles in the manufacture of a kit for modifying lymphocytes, in illustrative embodiments T cells and/or NK cells of a subject, wherein the use of the kit comprises the above method of transducing, genetically modifying, and/or modifying lymphocytes in whole blood. In another aspect, provided herein are methods for administering modified lymphocytes to a subject, wherein the modified lymphocytes are produced by the above method of transducing, genetically modifying, and/or modifying lymphocytes in whole blood. Aspects provided herein that include such methods of transducing, genetically modifying, and/or modifying lymphocytes in whole blood, uses of such a method in the manufacture of a kit, reaction mixtures formed in such a method, cell formulations made by such methods, modified lymphocytes made by such a method, and methods for administering a modified and in illustrative embodiments genetically modified lymphocyte made by such a method, are referred to herein as “composition and method aspects for transducing lymphocytes in whole blood.” It should be noted that although illustrative embodiments for such aspects involve contacting T cells and/or NK cells with retroviral particles in whole blood, such aspects also include other embodiments, where one or more of additional components a-f above, are present in transduction reaction mixtures at higher concentrations than is typical after a PBMC enrichment procedure. For example, such aspects arise when blood is fractionated using a filter that separates blood into components that include T cells and/or NK cells and additional blood components that are not present in PBMC preparations, for example the use of leukoreduction filters and the resulting presence of neutrophils in the cell-fraction that includes T cells and NK cells that is retained by the filter.

Various elements or steps of such method aspects for transducing lymphocytes in whole blood and reaction mixtures that include whole blood or one or more components thereof, are provided herein, for example in this section and the Exemplary Embodiments section, and such methods include embodiments that are provided throughout this specification, as further discussed herein. A skilled artisan will recognize that many embodiments provided herein anywhere in this specification can be applied to any of the aspects of the composition and method aspects for transducing lymphocytes in whole blood. For example, embodiments of any of the composition and method aspects for transducing lymphocytes in whole blood provided for example in this section and/or in the Exemplary Embodiments section, can include any of the embodiments of replication incompetent recombinant retroviral particles provided herein, including those that include one or more polypeptide lymphoproliferative element, inhibitory RNA, CAR, pseudotyping element, riboswitch, activation element, membrane-bound cytokine, miRNA, Kozak-type sequence, WPRE element, triple stop codon, and/or other element disclosed herein, and can be combined with methods herein for producing retroviral particles using a packaging cell. Furthermore, any aspect and embodiment of the composition (e.g., reaction mixture) and method aspects for transducing lymphocytes in whole blood, can be combined with any composition and method aspect including a self-driving CAR provided herein. Details regarding any composition and method aspects including a self-driving CAR are disclosed in more detail herein, for example in the Self-Driving CAR Methods and Compositions section and in the Exemplary Embodiments section.

In certain illustrative embodiments, the retroviral particle is a lentiviral particle. Such a method for modifying and in illustrative embodiments genetically modifying a lymphocyte, such as a T cell and/or NK cell in whole blood, can be performed in vitro or ex vivo.

Anticoagulants are included in reaction mixtures for certain embodiments of the composition (e.g., reaction mixtures) and method aspects for transducing lymphocytes in whole blood provided herein. In some illustrative embodiments, blood is collected with the anti-coagulant present in the collection vessel (e.g., tube or bag), for example using standard blood collection protocols known in the art. Anticoagulants that can be used in composition and method aspects for transducing lymphocytes in whole blood provided herein include compounds or biologics that block or limit the thrombin blood clotting cascade. The anticoagulants include: metal chelating agents, preferably calcium ion chelating agents, such as citrate (e.g., containing free citrate ion), including solutions of citrate that contain one or more components such as citric acid, sodium citrate, phosphate, adenine and mono or polysaccharides, for example dextrose, oxalate, and EDTA; heparin and heparin analogues, such as unfractionated heparin, low molecular weight heparins, and other synthetic saccharides; and vitamin K antagonists such as coumarins. Exemplary citrate compositions include: acid citrate dextrose (ACD) (also called anticoagulant citrate dextrose solution A and solution B (United States Pharmacopeia 26, 2002, pp 158)); and a citrate phosphate dextrose (CPD) solution, which can also be prepared as CPD-A1 as is known in the art. Accordingly, the anticoagulant composition may also include phosphate ions or monobasic phosphate ion, adenine, and mono or polysaccharides.

Such anticoagulants can be present in a reaction mixture at concentrations that are effective for preventing coagulation of blood (i.e., effective amounts) as known in the art, or at a concentration that is, for example, 2 times, 1.5 times, 1.25 times, 1.2 times, 1.1 times, or 9/10, ⅘, 7/10, ⅗, ½, ⅖, 3/10, ⅕, or 1/10 the effective concentration. The effective concentrations of many different anticoagulants are known and can be readily determined empirically by analyzing different concentrations for their ability to prevent blood coagulation, which can be physically observed. Numerous coagulometers are available commercially that measure coagulation, and various sensor technologies can be used, for example QCM sensors (See e.g., Yao et al., “Blood Coagulation Testing Smartphone Platform Using Quartz Crystal Microbalance Dissipation Method,” Sensors (Basel). 2018 Sep.; 18(9): 3073). The effective concentration includes the concentration of any commercially available anticoagulant in a commercially available tube or bag after the anticoagulant is diluted in the volume of blood intended for the tube or bag. For example, the concentration of acid citrate dextrose (ACD) in a reaction mixture in certain embodiments of the composition and method aspects for transducing lymphocytes in whole blood provided herein, can be between 0.1 and 5×, or between 0.25 and 2.5×, between 0.5 and 2×, between 0.75 and 1.5×, between 0.8 and 1.2×, between 0.9 and 1.1×, about 1×, or 1× the concentration of ACD in a commercially available ACD blood collection tube or bag. For example, in a standard process, blood can be collected into tubes or bags containing 3.2% (109 mM) sodium citrate (109 mM) at a ratio of 9 parts blood and 1 part anticoagulant. Thus, in certain illustrative embodiments with a reaction mixture made by adding 1-2 parts of a retroviral particle solution to this mixture of 1 part anticoagulant to 9 parts blood, the citrate concentration can be between for example, 25% to 0.4%, or 0.30% to 0.35%. In an illustrative standard blood collection embodiment, 15 ml of ACD Solution A are present in a blood bag for collecting 100 mL of blood. The ACD before addition of blood contains Citric acid (anhydrous) 7.3 g/L (0.73%), Sodium citrate (dihydrate) 22.0 g/L (2.2%), and Dextrose (monohydrate) 24.5 g/L [USP] (2.4%). After addition of 100 ml of blood to the bag that contains ACD, a volume of for example, between 5 and 20 ml of the retroviral particles is added. Thus, in some embodiments, the concentration of ACD components in a reaction mixture can be between 0.05 and 0.1%, or 0.06 and 0.08% Citric acid (anhydrous), 0.17 and 0.27, or 0.20 and 0.24 Sodium citrate (dihydrate), 0.2 and 0.3, or 0.20 and 0.28, or 0.22 and 0.26% Dextrose (monohydrate). In certain embodiments, sodium citrate is used at a concentration of between 0.001 and 0.02 M in the reaction mixture.

In some embodiments, heparin is present in the reaction mixtures, for example at a concentration between 0.1 and 5×, or between 0.25 and 2.5×, between 0.5 and 2×, between 0.75 and 1.5×, between 0.8 and 1.2×, between 0.9 and 1.1×, about 1×, or 1× the concentration of heparin in a commercially available heparin blood collection tube. Heparin is a glycosaminoglycan anticoagulant with a molecular weight ranging from 5,000-30,000 daltons. In some embodiments, heparin is used at a concentration of about 1.5 to 45, 5 to 30, 10 to 20, or 15 USP units/ml of reaction mixture. In some embodiments, the effective concentration for EDTA, for example as K₂EDTA, in the reaction mixtures herein can be between 0.15 and 5 mg/ml, between 1 and 3 mg/ml between 1.5-2.2 mg/ml of blood, or between 1 and 2 mg/ml, or about 1.5 mg/ml. The reaction mixtures in composition and method aspects for transducing lymphocytes in whole blood provided herein, can include two or more anticoagulants whose combined effective dose prevents coagulation of the blood prior to formation of the reaction mixture and/or of the reaction mixture itself.

In some embodiments, the anticoagulant can be administered to a subject before blood is collected from the subject for ex vivo transduction, such that coagulation of the blood when it is collected in inhibited, at least partially and at least through a contacting step and optional incubation period thereafter. In such embodiments, for example acid citrate dextrose can be administered to the subject at between 80 mg/kg/day and 5 mg/kg/day (mg refer to the mg of citric acid and kg applies to the mammal to be treated). Heparin, can be delivered for example, at a dose of between 5 units/kg/hr to 30 units/kg/hr.

Reaction mixtures in certain illustrative embodiments herein can include blood or blood preparation component that is not a PBMC, as provided herein. Non-limiting exemplary concentrations of such components are provided in the following paragraphs. It will be understood that resulting cell formulations from methods using these reaction mixtures, in illustrative embodiments will include these additional components, and in some embodiments at the same ratios or percentages relative to other cells, provided below for reaction mixtures.

With respect to erythrocytes, in some embodiments, erythrocytes are present in reaction mixtures and cell formulations herein, in some embodiments at a relative amount to T cells that is greater than after a typical PBMC isolation, and in some embodiments, erythrocytes can comprise between 0.1, 0.5, 1, 5, 10, 25, 35 or 40% of the volume of the reaction mixture on the low end of the range, and between 25, 50, 60, or 75% of the volume of the reaction mixture on the high end of the range. In illustrative embodiments, erythrocytes comprise between 1 and 60%, between 10 and 60%, between 20 and 60%, between 30 and 60%, between 40 and 60%, between 40 and 50%, between 42 and 48%, between 44 and 46%, about 45% or 45% of the reaction mixture. In some embodiments, more erythrocytes are present than T cells in a reaction mixture or cell formulation.

With respect to neutrophils, in some embodiments neutrophils are present in reaction mixtures and cell formulations provided herein, in some embodiments at a relative amount to T cells that is greater than after a typical PBMC isolation, and in some embodiments, neutrophils can comprise between 0.1, 0.5, 1, 5, 10, 20, 25, 35 or 40% of the white blood cells of the reaction mixture or cell formulation on the low end of the range, and between 25, 50, 60, 70, 75 and 80% of the white blood cells of the reaction mixture or cell formulation on the high end of the range, for example between 25% and 70%, or between 30% and 60%, or between 40% and 60% of the white blood cells of the reaction mixture or cell formulation. In some embodiments, more neutrophils are present than T cells and/or NK cells, in reaction mixtures and cell formulations herein.

With respect to eosinophils, eosinophils are present in a reaction mixture or a cell formulation, in some embodiments at a relative amount to T cells that is greater than after a typical PBMC isolation, and in some embodiments eosinophils can comprise between 0.05, 0.1, 0.2, 0.4, 0.6, 0.8, 1.0, 1.2, 1.4, 1.6, and 1.8% of the white blood cells of the reaction mixture or cell formulation on the low end of the range, and between 2.0, 2.2, 2.4, 2.6, 2.8, 3.0, 3.5, 4, 5, 6, 8 and 10% of the white blood cells of the reaction mixture or cell formulation on the high end of the range. In illustrative embodiments, eosinophils comprise between 0.05 and 10.0%, between 0.1 and 9%, between 0.2 and 8%, between 0.2 and 6%, between 0.5 and 4%, between 0.8 and 4%, or between 1 and 4% of the white blood cells of the reaction mixture or cell formulation.

With respect to basophils, in some embodiments basophils are present in a reaction mixture or cell formulation, in some embodiments at a relative amount to T cells that is greater than after a typical PBMC isolation, and in some embodiments basophils can comprise between 0.05, 0.1, 0.2, 0.4, 0.45, and 0.5% of the white blood cells of the reaction mixture on the low end of the range, and between 0.8, 0.9, 1.0, 1.1, 1.2, 1.5, and 2.0% of the white blood cells of the reaction mixture on the high end of the range. In illustrative embodiments, basophils comprise between 0.05 and 1.4%, between 0.1 and 1.4%, between 0.2 and 1.4%, between 0.3 and 1.4%, between 0.4 and 1.4%, between 0.5 and 1.4%, between 0.5 and 1.2%, between 0.5 and 1.1%, or between 0.5 and 1.0% of the white blood cells of the reaction mixture.

With respect to plasma, in some embodiments, plasma components are present in a reaction mixture or cell formulation, and in some embodiments, plasma can comprise between 0.1, 0.5, 1, 5, 10, 25, 35 or 45% of the volume of the reaction mixture on the low end of the range, and between 25, 50, 60, 70 and 80% of the volume of the reaction mixture on the high end of the range. In illustrative embodiments, plasma comprise between 0.1 and 80%, between 1 and 80%, between 5 and 80%, between 10 and 80%, between 30 and 80%, between 40 and 80%, between 45 and 70%, between 50 and 60%, between 52 and 58%, between 54 and 56%, about 55% or 55% of the reaction mixture. With respect to platelets, in some embodiments, platelets are present in a reaction mixture or cell formulation, in some embodiments at a relative amount to T cells that is greater than after a typical PBMC isolation, and in some embodiments they can comprise between 1×10⁵, 1×10⁶, 1×10⁷, or 1×10⁸ platelets/mL of the reaction mixture on the low end of the range, and between 1×10⁹, 1×10¹⁰, 1×10¹¹, 1×10¹², 2×10¹³, or 2×10¹⁴ platelets/ml of the reaction mixture on the high end of the range. In illustrative embodiments, platelets comprise between 1×10⁵ and 1×10¹² platelets, between 1×10⁶ and 1×10¹¹ platelets, between 1×10⁷ and 1×10¹⁰ platelets, between 1×10⁸, and 1×10⁹ platelets/ml, or between 1×10⁸ and 5×10⁸ platelets/ml of the reaction mixture., in some embodiments at a relative amount to T cells that is greater than after a typical PBMC isolation, and in some embodiments at between 0.1% and 9%, 0.1% and 1%, or between 1% and 9% of white blood cells in the reaction mixture or cell formulation.

### Steps and Reaction Mixtures for Methods for Modifying and/or Genetically Modifying Lymphocytes

Provided herein in certain aspects, is a method of transducing, transfecting, genetically modifying, and/or modifying a lymphocyte, such as a (typically a population of) peripheral blood mononuclear cell (PBMC), typically a T cell and/or an NK cell, and in certain illustrative embodiments a resting T cell and/or resting NK cell, that includes contacting the lymphocyte with a (typically a population of) recombinant nucleic acid vector, which in illustrative embodiments is a replication incompetent recombinant retroviral particle, wherein said contacting (and incubation under contacting conditions) facilitates membrane association, membrane fusion or endocytosis, and optionally transduction or transfection of the resting T cell and/or NK cell by the recombinant nucleic acid vector, thereby producing the modified and in illustrative embodiments genetically modified T cell and/or NK cell. It is noteworthy that although many of the aspects and embodiments provided herein are discussed in terms of a recombinant retroviral particle, it is intended, and a skilled artisan will recognize, that many different recombinant nucleic acid vectors, including but not limited to those provided herein, can be used and/or included in such methods and compositions. In illustrative embodiments wherein the recombinant nucleic acid vector is a replication incompetent recombinant retroviral particle, the replication incompetent recombinant retroviral particle typically comprises a fusogenic element and a binding element, which can be part of a pseudotyping element, on its surface. In illustrative embodiments, pre-activation of the T cell and/or NK cell is not required, and an activation element, which can be any activation element provided herein, is present in a reaction mixture in which the contacting takes place. In further illustrative embodiments, the activation element is present on a surface of the replication incompetent recombinant retroviral particle. In illustrative embodiments, the activation element is anti-CD3, such as anti-CD3 scFv, or anti-CD3 scFvFc.

Many of the method aspects provided herein, include the following steps: 1) an optional step of collecting blood from a subject; 2) a step of contacting cells, such as NK cells and/or in illustrative embodiments T cells, which can be from the collected blood, with a recombinant vector (typically many copies thereof), in illustrative embodiments a replication incompetent recombinant retroviral particle, encoding a CAR and/or a lymphoproliferative element, in a reaction mixture, where the contacting can include an optional incubation; 3) typically a step of washing unbound recombinant vector away from the cells in the reaction mixture; 4) typically a step of collecting modified cells, such as modified NK cells and/or in illustrative embodiments modified T cells in a solution, which in illustrative embodiments can be a delivery solution, to form a cell suspension, that in illustrative embodiments is a cell formulation; and 5) an optional step of delivering the cell formulation to a subject, in illustrative embodiments the subject from which blood was collected, for example through infusion, or in certain illustrative embodiments intradermally, intramuscularly or intratumorally, or in further illustrative embodiments, subcutaneously. It is noteworthy that in certain illustrative embodiments, the reaction mixture includes unfractionated whole blood or includes one or more cell type that is not a PBMC, and can include all or many cell types found in whole blood, including total nucleated cells (TNCs). It is noteworthy that in certain embodiments, the recombinant vector comprises a self-driving CAR, which encodes both a CAR and a lymphoproliferative element.

As a non-limiting example, in some embodiments, between 10 and 120 ml of blood is collected (or leukocytes are isolated in 10 to 120 ml by performing leukapheresis on 0.5 to 2.0 total blood volumes); the collected, unfractionated blood/isolated cells are passed through a leukoreduction filter to isolate TNCs on top of the filter; replication incompetent recombinant retroviral particles are added to the TNCs on top of the leukoreduction filter to a total reaction mixture volume of 500 μl to 10 ml to form a reaction mixture and initiate contacting; the reaction mixture is optionally incubated for any of the contacting times provided herein, as a non-limiting example, for 1-4 hours; the non-associated replication incompetent recombinant retroviral particles are washed away from cells in the reaction mixture by filtering the reaction mixture with 10 to 120 ml of wash solution; and the cells, including modified T cells and NK cells, which are retained on the TNC filter, are eluted from the filter with 2 ml to 10 ml of delivery solution, thereby forming a cell formulation suitable for introduction or reintroduction into a subject.

Some embodiments of any methods used in any aspects provided herein, which are typically methods for modifying and in illustrative embodiments genetically modifying lymphocytes, PBMCs, and in illustrative embodiments NK cells and/or in further illustrative embodiments, T cells, can include a step of collecting blood from a subject. The blood includes blood components including blood cells such as lymphocytes (e.g., T cells and NK cells) that can be used in methods and compositions provided herein. In certain illustrative embodiments, the subject is a human subject afflicted with cancer (i.e., a human cancer subject). It is noteworthy that certain embodiments do not include such a step. However, in embodiments that include collecting blood from a subject, blood can be collected or obtained from a subject by any suitable method known in the art as discussed in more detail herein, and as such the collected blood or blood-derived component can be referred to as a “blood-derived product” and typically is a “peripheral blood-derived product,” since typically it is isolated from peripheral blood. For example, the blood-derived product can be collected by venipuncture or any other blood collection method known in the art, by which a sample of unfractionated whole blood is collected in a vessel, for example a blood bag, or by which leukocytes and lymphocytes are isolated from blood, such as by apheresis (e.g., leukapheresis or lymphoplasmapheresis). In some embodiments, the volume of blood (e.g., unfractionated whole blood) collected is between 1 and 5 ml, 5 and 10 ml, 10 and 15 ml, 15 and 20 ml, 20 and 25 ml, 5 and 25 ml, 25 ml and 250 ml, 25 ml and 125 ml, 50 ml and 100 ml, or 50 ml and 250 ml, 75 ml and 125 ml, 90 ml and 120 ml, or between 95 and 110 ml. In some embodiments, the volume of blood collected can be between 1, 5, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml on the low end of the range and 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml or 1 L on the high end of the range. In some embodiments, the volume of blood collected is less than 250 ml, 100 ml, 75 ml, 20 ml, 15 ml, 10 ml, or 5 ml. In some embodiments, lymphocytes (e.g., T cells and/or NK cells) can be obtained by apheresis. In some embodiments, the volume of blood taken and processed during apheresis (e.g., leukapheresis or lymphoplasmapheresis) is between 0.5, 0.6, 0.7, 0.75, 0.8, 0.9, 1, 1.25, or 1.5 total blood volumes of a subject on the low end of the range and 0.6, 0.7, 0.75, 0.8, 0.9, 1, 1.25, 1.5 1.75, 2, 2.25, or 2.5 total blood volumes of a subject on the high end of the range, for example between 0.5 and 2.5, 0.5 and 2, 0.5 and 1.5, or between 1 and 2 total blood volumes. The total blood volume of a human typically ranges from 4.5 to 6 L and thus much more blood is typically taken and processed during apheresis than if unfractionated whole blood is collected. Whether target blood cells (e.g., T cells) are obtained by apheresis or unfractionated whole blood is collected for example into a blood bag, it is contemplated that target blood cells (e.g., T cells) therein would be processed according to a method provided herein, which in certain illustrative embodiments results in the target blood cells becoming modified, genetically modified, and/or transduced. When apheresis (e.g., leukapheresis or lymphoplasmapheresis) is used to collect a cell fraction comprising T cells and/or NK cells (e.g., to provide a leukopak or a lymphoplasmapak), such cells are resuspended in solution directly or after one or more washes, to which a recombinant vector encoding a CAR is added to form a reaction mixture provided herein. Such reaction mixture can be used in any method herein. In some illustrative methods where a subject or a blood sample therefrom has a low CD3+ blood cell count, apheresis (e.g., leukapheresis or lymphoplasmapheresis) is used to collect blood cells (e.g., White blood cells or lymphocytes) for inclusion in a method provided herein.

Regardless of whether blood is collected from a subject or blood cells are obtained by apheresis, in any of the method aspects provided herein for modifying lymphocytes (e.g. T cells and/or NK cells), a population of lymphocytes (e.g. T cells and/or NK cells) are typically contacted with many copies of a recombinant vector, which in some embodiments are copies of a non-viral vector, and in illustrative embodiments are identical replication incompetent recombinant retroviral particles, in a reaction mixture. The contacting in any embodiment provided herein, can be performed for example in a chamber of a closed system adapted for processing of blood cells, for example within a blood bag, as discussed in more detail herein. In some embodiments, the blood bag can have 5, 10, 15, 20, 25, 50, 75, 100, 150, 200, 250, 300, 400, or 500 ml or less of blood during the contacting. In some embodiments, the blood bag can have at least 5, 10, 15, 20, 25, 50, 75, 100, 150, 200, 250, 300, 400, or 500 ml of blood during the contacting. In some embodiments, the blood bag can have between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range during the contacting. For example, the blood bag can have between 1 and 10 ml, 5 and 25 ml, 10 and 50 ml, 25 and 100 ml, 50 and 200 ml, or 100 and 500 ml of blood during the contacting. In some embodiments, the mixture inside the blood bag can include an anti-coagulant such as heparin. In other embodiments, the mixture inside the blood bag does not include an ant-coagulant, or does not include heparin. The transduction reaction mixture can include one or more buffers, ions, and a culture media.

With respect to retroviral particles, and in illustrative embodiments, lentiviral particles, in certain exemplary reaction mixtures provided herein, between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15, multiplicity of infection (MOI); or at least 1 and less than 6, 11, or 51 MOI; or in some embodiments, between 5 and 10 MOI units of replication incompetent recombinant retroviral particles are present. In some embodiments, the MOI can be at least 0.1, 0.5, 1, 2, 2.5, 3, 5, 10 or 15. With respect to composition and method for transducing lymphocytes in blood, in certain embodiments higher MOI can be used than in methods wherein PBMCs are isolated and used in the reaction mixtures. For example, illustrative embodiments of compositions and methods for transducing lymphocytes in whole blood, assuming 1×10⁶ PBMCs/ml of blood, can use retroviral particles with an MOI of between 1 and 50, 2 and 25, 2.5 and 20, 2.5 and 10, 4 and 6, or about 5, and in some embodiments between 5 and 20, 5 and 15, 10 and 20, or 10 and 15.

As noted in Example 812 herein, surface expression of the TCR complex, including TCRα, TCRβ, and CD3, on CD4 positive (CD4+) cells and CD8 positive (CD8+) cells is reduced or “dimmed” when such cells are contacted with gene vectors (e.g., replication incompetent recombinant (RIR) retroviral particles) displaying a binding polypeptide that binds the TCR complex, e.g., a T cell activation element. This dimming is largely the result of internalization of the TCR complex upon activation. Furthermore, the extent of this dimming increases as the concentration of a given gene vector is increased in the reaction mixture and correlates with the ability of the gene vector to activate and enter cells Similarly, internalization of other surface polypeptides after binding to polypeptides on the surface of a gene vector results in dimming of the surface polypeptide on the cell being contacted with the gene vector and may be common during transduction using other binding polypeptides. Thus, in some embodiments, a percent reduction in surface polypeptide expression on cells contacted with a gene vector comprising a binding polypeptide compared to surface polypeptide expression on cells not contacted with the gene vector comprising a binding polypeptide is used to quantitate the potency of a gene vector and determine the appropriate dose of gene vector used to modify a population of cells. In illustrative embodiments, a percent reduction in surface TCR complex expression on cells contacted with a gene vector compared to surface TCR complex expression on cells not contacted with the gene vector is used to quantitate the potency of a gene vector and determine the appropriate dose of gene vector used to modify a population of cells. As used herein, a “Dimming Unit” (DU) is the amount of gene vector (e.g. RIR retroviral particles) that reduces the surface expression of a surface polypeptide in 1 ml of a cell mixture after contacting with the gene vector for 4 hours at 37° C. and 5% CO₂ by 50% compared to the surface expression of the surface polypeptide in the cell mixture under similar conditions but not contacted with the gene vector. The surface polypeptide is typically a binding partner of a binding polypeptide present on the surface of the gene vector. In some embodiments, the surface polypeptide is a TCR complex polypeptide. In some embodiments, the TCR complex polypeptide is CD3D, CD3E, CD3G, CD3Z, TCRα, or TCRβ. In illustrative embodiments, the binding partner is CD3 and the binding polypeptide is anti-CD3.

Because the level of expression of a binding polypeptide on the surface of the gene vector will vary between different binding polypeptides and between gene vector preparations, the ability of a gene vector to reduce surface expression of a surface polypeptide should be determined for each preparation of a gene vector. In some embodiments, the ability of a gene vector to reduce surface expression of a surface polypeptide is determined based on target cell number. In some embodiments, the ability of a gene vector to reduce surface expression of a surface polypeptide is based on the volume the cells. In any of the aspects and embodiments herein, the reduction of surface expression of a surface polypeptide can be referred to as dimming the surface polypeptide. For example, if the surface expression of CD3 on a cell is reduced, then CD3 is dimmed on that cell and the cell can be called CD3−, even though the cell may still contain CD3 not expressed on its surface. Not to be limited by theory, T cells that temporarily internalize and dim CD3 are T cells and will eventually re-express CD3 on their cell surfaces such that they are again CD3+.

Accordingly, provided herein in one aspect, is a method for determining an amount of a gene vector preparation to dim surface expression of a surface polypeptide by a dimming percentage on cells in a dimming volume, comprising:

a) forming a plurality of reaction mixtures comprising a plurality of volumes of the gene vector preparation and a plurality of volumes of a cell mixture, wherein at least two of the reaction mixtures in the plurality of reaction mixtures comprise different volumes of the gene vector preparation and/or the cell mixture, wherein the cell mixture comprises a plurality of cells comprising the surface polypeptide on their surfaces, and wherein the gene vector preparation comprises a plurality of gene vectors comprising a binding polypeptide on their surfaces capable of binding the surface polypeptide;

b) incubating the reaction mixtures;

c) measuring the surface expression of the surface polypeptide in the reaction mixtures and in an uncontacted volume of the cell mixture, wherein the uncontacted volume of the cell mixture is not contacted with the gene vector preparation; and

d) determining the amount of the gene vector preparation to dim the dimming percentage of cells in the dimming volume the measured surface expression of the surface polypeptide in the reaction mixtures, the measured surface expression of the surface polypeptide in the uncontacted volume of the cell mixture, and the amounts of the gene vector preparation and the cell mixture in the reaction mixtures.

In some embodiments, the amounts of the cell mixture in the reaction mixtures is based on volume. In some embodiments, amounts of the cell mixture in the reaction mixtures is based on numbers of target cells. In some embodiments, the gene vector preparation is a viral preparation. In illustrative embodiments, the viral preparation is a replication incompetent recombinant retroviral particle preparation. In some embodiments, the dimming percentage (percentage of cells dimmed) is 50%, 60%, 70%, 75%, 80%, 85%, 90%, 95%, 96%, or 97%. In illustrative embodiments, the dimming percentage is at least or about 80%, 85%, 90%, or 95%. In some embodiments, the dimming volume is 0.25 ml, 0.5 ml, 0.75 ml, 1 ml, 2 ml, 3 ml, 4 ml, 5 ml, 10 ml, 15 ml, 20 ml, or 25 ml. In some embodiments, the surface polypeptide can be CD3D, CD3E, CD3G, CD3Z, TCRα, TCRβ, CD16A, NKp46, 2B4, CD2, DNAM, or NKG2C, NKG2D, NKG2E, NKG2F, and/or NKG2H. In some embodiments, the surface polypeptide is a TCR complex polypeptide. In some embodiments, the TCR complex polypeptide is CD3D, CD3E, CD3G, CD3Z, TCRα, or TCRβ. In illustrative embodiments, the surface polypeptide is CD3E. In some embodiments, the binding polypeptide can be any of the activation elements disclosed in the Activation Elements section herein. In such embodiments, the surface polypeptide can be the binding partner of the activation element.

In illustrative embodiments, the cell mixture is whole blood. In further illustrative embodiments, the cell mixture has been subjected to a red blood cell depletion procedure. In some embodiments, the whole blood is collected from a healthy subject, e.g., a subject that does not have or is not known or suspected to have a disease, disorder, or condition associated with associated with an elevated expression of an antigen. In some embodiments, the whole blood is collected from a subject with a disease, disorder, or condition associated with associated with an elevated expression of an antigen, wherein the gene vector will be administered to the subject or other subjects with the disease disorder, or condition. In some embodiments, the whole blood is collected from each subject and the Dimming Units are calculated for each subject individually.

In some embodiments, the reaction mixtures can be incubated for less than or about 24, 12, 10, 8, 6, 4, or 2 hours or 60, 45, 30, 15, 10, or 5 minutes, or for just an initial contacting. In some embodiments, the reaction mixtures can be incubated for between 10 minutes and 24 hours, or between 10 minutes and 8 hours, or for between 1 hour and 8 hours, or for between 1 hour and 6 hours, or in illustrative embodiments, for between 3.5 and 4.5 hours or for 4 hours. In some embodiments, the reaction mixtures can be incubated at about 10° C., 15° C., 20° C., 25° C., 30° C., 37° C., or 42° C. In some embodiments, the reaction mixtures are incubated without CO₂. In illustrative embodiments, the reaction mixtures are incubated with 5% CO₂.

In some embodiments, the surface expression of the surface polypeptide is measured by fluorescence-activated cell sorting (FACS) method. In some embodiments, the antibody used in a FACS method is GMP. In some embodiments, a CD3 antibody is used to determine surface expression of the surface polypeptide. In some embodiments, the CD3 antibody is UCHT1, OKT-3, HIT3A, TRX4, X35-3, VIT3, BMA030 (BW264/56), CLB-T3/3, CRIS7, YTH12.5, F111409, CLB-T3.4.2, TR-66, TR66.opt, HuM291, WT31, WT32, SPv-T3b, 11D8, XIII-141, XII146, XIII-87, 12F6, T3/RW2-8C8, T3/RW24B6, OKT3D, M-T301, SMC2, F101.01, and SK7. In illustrative embodiments, the CD3 antibody is PerCP Mouse Anti-Human CD3-Clone SK7 (BD, 347344). In some embodiments, before measuring the surface expression of the surface polypeptide, cells present in the cell mixture are separated from unbound gene vector in the incubated reaction mixture.

In an illustrative embodiment of the above method, the gene vector preparation is a replication incompetent recombinant retroviral particle preparation, the dimming percentage is 50%, the dimming volume is 1 ml, the surface polypeptide is CD3, the cell mixture is whole blood collected from a healthy subject, and the reaction mixture is incubated for 4 hours at 37° C. and 5% CO₂ and the method is used to calculate Dimming Units.

Such methods can be used to determine the amount of retroviral particles in a gene vector preparation that reduces surface polypeptide expression on cells by a specific percentage. This amount can then be used to determine an amount of the preparation of retroviral particles to use for subsequent transductions of whole blood, isolated PBMCs, or isolated TNCs. In any of the aspects and embodiments provided herein that include genetically modifying and/or transducing lymphocytes, the amount of a preparation of gene vector, for example replication incompetent recombinant retroviral particles, to add to the lymphocytes can be determined using the method above.

Dimming Units (DUs) can be used in any of the aspects or embodiments herein that include a contacting step to determine the amount of gene vector to add. As 1 DU of gene vector reduces the surface expression of the surface polypeptide by 50% in a 1 ml volume of cells, 10 DUs of gene vector reduces the surface expression of the surface polypeptide by 50% in 10 ml of a cell mixture. In some embodiments, sufficient DUs are added to a volume of cells to reduce surface expression of the surface polypeptide, for example CD3, by greater than 50%, 60%, 70%, 75%, 80%, 85%, 90%, 95%, 96%, or 97% after contacting with the gene vector compared to the surface expression of the surface polypeptide in the cell mixture under similar conditions but not contacted with the gene vector. In illustrative embodiments, sufficient DUs are added to a volume of cells to reduce surface expression of the surface polypeptide by greater than 80%, 85%, 90%, or 95% after contacting with the gene vector compared to the surface expression of the surface polypeptide in the cell mixture under similar conditions but not contacted with the gene vector. In some embodiments, at least 1, 2, 3, 4, 5, 6, 7, 8, 9, 10, 11,12, 13, 14, 15, 16, 17, 18, 19 or 20 DU are added per ml of cell mixture. In illustrative embodiments, between 5 and 20 DU, 5 and 15 DU, 10 and 20 DU, or 13 and 18 DU are added per ml of cell mixture. In some embodiments, at least 1, 2, 3, 4, 5, 6, 7, 8, 9, 10, 11,12, 13, 14, 15, 16, 17, 18, 19 or 20 DU are added per 1,000,000 target cells. In some embodiments, the target cells are lymphocytes, for example T cells or NK cells. In illustrative embodiments, the cells are in whole blood, isolated PBMCs, or isolated TNCs. In further illustrative embodiments, the cells are the remaining fraction of whole blood after lysing red blood cells. In some embodiments, sufficient DUs are added to dim a population of cells a specific percentage, for example, to dim CD3 on a population of T cells by greater than 50%, 60%, 70%, 75%, 80%, 85%, 90%, 95%, 96%, or 97%. In some embodiments, sufficient dimming units of a gene vector, and in illustrative embodiments RIP are present to increase the percentage of surface dimmed surface polypeptide, and in illustrative embodiments dimmed surface CD3−, in a population of cells, and in illustrative embodiments T cells, to at least 50%, 60%, 70%, 75%, 80%, 85%, 90%, 95%, 96%, or 97%. In any of the aspects and embodiments herein that include cells contacted with a gene vector, the composition including cells can include at least 1, 2, 3, 4, 5, 6, 7, 8, 9, 10, 11,12, 13, 14, 15, 16, 17, 18, 19 or 20 DU per ml of the cells, for example at least 1, 2, 3, 4, 5, 6, 7, 8, 9, 10, 11,12, 13, 14, 15, 16, 17, 18, 19 or 20 DU per ml of blood, cell formulation, population if cells.

In illustrative embodiments, this contacting, and the reaction mixture in which the contacting occurs, takes place within a closed cell processing system, as discussed in more detail herein. A packaging cell, and in illustrative embodiments a packaging cell line, and in particularly illustrative embodiments a packaging cell provided in certain aspects herein, can be used to produce the replication incompetent recombinant retroviral particles. The cells in the reaction mixture can be PBMCs or TNCs, and/or in reaction mixture aspects herein that provide compositions and methods for transducing lymphocytes in whole blood, an anticoagulant and/or an additional blood component, including additional types of blood cells that are not PBMCs, can be present as discussed herein. In fact, in illustrative embodiments of these composition and method aspects for transducing lymphocytes in whole blood, the reaction mixture can essentially be whole blood, and typically an anticoagulant, retroviral particles, and a relatively small amount of the solution in which the retroviral particles were delivered to the whole blood.

In reaction mixtures that relate to composition and method aspects for modifying lymphocytes in whole blood provided herein, lymphocytes, including NK cells and T cells, can be present at a lower percent of blood cells, and at a lower percentage of white blood cells, in the reaction mixture than methods that involve a PBMC enrichment procedure before forming the reaction mixture. For example, in some embodiments of these aspects, more granulocytes or neutrophils are present in the reaction mixture than NK cells or even T cells. Details regarding compositions of anticoagulants and one or more additional blood components present in the reaction mixtures of aspects for modifying lymphocytes in whole blood, are provided in detail in other sections herein. In some reaction mixture provided herein, T cells can be for example, between 10, 20, 30, or 40% of the lymphocytes of the reaction mixture on the low end of the range, and between 40, 50, 60, 70, 80, or 90% of the lymphocytes of the reaction mixture on the high end of the range. In illustrative embodiments, T cells comprise between 10 and 90%, between 20 and 90%, between 30 and 90%, between 40 and 90%, between 40 and 80%, or between 45% to 75% of the lymphocytes. In such embodiments, for example, NK cells can be present at between 1, 2, 3, 4, or 5% of the lymphocytes of the reaction mixture on the low end of the range, and between 5, 6, 7, 8, 9, 10, 11, 12, 13, or 14% of the lymphocytes of the reaction mixture on the high end of the range. In illustrative embodiments, T cells comprise between 1 and 14%, between 2 and 14%, between 3 and 14%, between 4 and 14%, between 5 and 14%, between 5 to 13%, between 5 to 12%, between 5 to 11% or between 5 to 10% of the lymphocytes of the reaction mixture. In some embodiments, T cells can be at least 1%, 2%, 3%, 4%, 5%, 6%, 7%, 8%, 9%, 10%, 15%, 20%, 30%, 40%, 50%, 60%, 70%, 80%, 90%, 95%, 96%, 97%, 98%, or 99% of the reaction mixture. As disclosed herein, composition and method aspects for transducing lymphocytes in whole blood typically do not involve any blood fractionation such as a PBMC enrichment step of a blood sample, before lymphocytes from the blood sample are contacted with recombinant nucleic acid vectors, for example retroviral particles, in the reaction mixtures disclosed herein for those aspects. Thus, in some embodiments, lymphocytes in unfractionated whole blood, are contacted with recombinant retroviral particles. However, in some embodiments, especially for some aspects in the Self-Driving CAR Methods and Compositions section herein, neutrophils/granulocytes are separated away from other blood cells before the cells are contacted with replication incompetent recombinant retroviral particles. In some embodiments, peripheral blood mononuclear cells (PBMCs) including peripheral blood lymphocytes (PBLs) such as T cell and/or NK cells, are isolated away from other components of a blood sample using for example, a PBMC enrichment procedure, before they are combined into a reaction mixture with retroviral particles. A skilled artisan will understand various methods known in the art can be used to enrich different blood fractions containing T cells and/or NK cells.

A PBMC enrichment procedure is a procedure in which PBMCs are enriched at least 25-fold, and typically at least 50-fold from other blood cell types. For example, it is believed that PBMCs make up less than 1% of blood cells in whole blood. After a PBMC enrichment procedure, at least 30%, and in some examples as many as 70% of cells isolated in the PBMC fraction are PBMCs. It is possible that even higher enrichment of PBMCs is achieved using some PBMC enrichment procedures. Various different PBMC enrichment procedures are known in the art. For example, a PBMC enrichment procedure is a ficoll density gradient centrifugation process that separates the main cell populations, such as lymphocytes, monocytes, granulocytes, and red blood cells, throughout a density gradient medium. In such a method the aqueous medium includes ficoll, a hydrophilic polysaccharide that forms the high density solution. Layering of whole blood over or under a density medium without mixing of the two layers followed by centrifugation will disperse the cells according to their densities with the PBMC fraction forming a thin white layer at the interface between the plasma and the density gradient medium (see e.g., Panda and Ravindran (2013) Isolation of Human PBMCs. BioProtoc. Vol. 3(3)). Furthermore, centripetal forces can be used to separate PBMCs from other blood components, in ficoll using the spinning force of a Sepax cell processing system.

In some embodiments, apheresis, for example leukapheresis, can be used to isolate cells, such as PBMCs. For example, AMICUS RBCX (Fresenius-Kabi) and Trima Accel (Terumo BCT) apheresis devices and kits can be used. Cells isolated by apheresis typically contain T cells, B cells, NK cells, monocytes, granulocytes, other nucleated white blood cells, red blood cells, and/or platelets. The cells collected by apheresis can be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. In some embodiments, the cells collected by apheresis can be genetically modified by any of the methods provided herein. In some embodiments, the cells collected by apheresis can be used to prepare any of the cell formulations provided herein. In some embodiments, the cells collected by apheresis can be resuspended in a variety of biocompatible buffers, such as, for example, Ca-free, Mg-free PBS. Alternatively, the undesirable components of the sample containing the cells collected by apheresis can be removed and the cells resuspended in culture media. In some embodiments, leukopheresis can be used to isolate cells, such as lymphocytes. In any of the embodiments provided herein that includes PBMCs, a leukopak can be used. In any embodiment that includes TNCs, a buffy coat can be used. In another PBMC enrichment method, an automated leukapheresis collection system (such as SPECTRA OPTIA® APHERESIS SYSTEM from Terumo BCT, Inc. Lakewood, Colo. 80215, USA) is used to separate the inflow of whole blood from the target PBMC fraction using high-speed centrifugation while typically returning the outflow material, such as plasma, red blood cells, and granulocytes, back to the donor, although this returning would be optional in methods provided herein. Further processing may be necessary to remove residual red blood cells and granulocytes. Both methods include a time intensive purification of the PBMCs, and the leukapheresis method requires the presence and participation of the patient during the PBMC enrichment step.

As further non-limiting examples of PBMC enrichment procedures, in some embodiments for methods of transducing, genetically modifying, and/or modifying herein, PBMCs are isolated using a Sepax or Sepax 2 cell processing system (BioSafe). In some embodiments, the PBMCs are isolated using a CliniMACS Prodigy cell processor (Miltenyi Biotec). In some embodiments, an automated apheresis separator is used which takes blood from the subject, passes the blood through an apparatus that sorts out a particular cell type (such as, for example, PBMCs), and returns the remainder back into the subject. Density gradient centrifugation can be performed after apheresis. In some embodiments, the PBMCs are isolated using a leukoreduction filter assembly. In some embodiments, magnetic bead activated cell sorting is then used for purifying a specific cell population from PBMCs, such as, for example, PBLs or a subset thereof, according to a cellular phenotype (i.e., positive selection), before they are used in a reaction mixture herein.

Other methods for purification can also be used, such as, for example, substrate adhesion, which utilizes a substrate that mimics the environment that a T cell encounters during recruitment, to purify T cells before adding them to a reaction mixture, or negative selection can be used, in which unwanted cells are targeted for removal with antibody complexes that target the unwanted cells for removal before a reaction mixture for a contacting step is formed. In some embodiments, red blood cell rosetting can be used to remove red blood cells before forming a reaction mixture. In other embodiments, hematopoietic stem cells can be removed before a contacting step, and thus in these embodiments, are not present during the contacting step. In some embodiments herein, especially for compositions and methods for transducing lymphocytes in whole blood, an ABC transporter inhibitor and/or substrate is not present before, during, or both before and during the contacting (i.e., not present in the reaction mixture in which contacting takes place) with or without optional incubating, or any step of the method.

In certain illustrative embodiments for any aspects provided herein, lymphocytes are modified and in illustrative embodiments genetically modified and/or transduced without prior activation or stimulation, and/or without requiring prior activation or stimulation, whether in vivo, in vitro, or ex-vivo; and/or furthermore, in some embodiments, without ex vivo or in vitro activation or stimulation after an initial contacting with or without an optional incubation, or without requiring ex vivo or in vitro activation or stimulation after an initial contacting with or without an optional incubation. In certain illustrative embodiments, the cell is activated during the contacting and is not activated at all or not activated for more than 15 minutes, 30 minutes, 1, 2, 4, or 8 hours before the contacting. In certain illustrative embodiments, activation by elements that are not present on the retroviral particle surface is not required for modifying, genetically modifying, and/or transducing the cell. Accordingly, such activation or stimulation elements are not required other than on the retroviral particle, before, during, or after the contacting. Thus, as discussed in more detail herein, these illustrative embodiments that do not require pre-activation or stimulation provide the ability to rapidly perform in vitro experiments aimed at better understanding T cells and the biologicals mechanisms, therein. Furthermore, such methods provide for much more efficient commercial production of biological products produced using PBMCs, lymphocytes, T cells, or NK cells, and development of such commercial production methods. Finally, such methods provide for more rapid ex vivo processing of lymphocytes (e.g., NK cells and especially T cells) for adoptive cell therapy, fundamentally simplifying the delivery of such therapies, for example by providing rapid point-of-care (rPOC) methods. In illustrative embodiments, some, most, at least 25%, 50%, 60%, 70%, 75%, 80%, 90%, 95%, or 99%, or all of the lymphocytes are resting when they are combined with retroviral particles to form a reaction mixture, and typically are resting when they are contacted with retroviral viral particles in a reaction mixture. In methods for modifying lymphocytes such as T cells and/or NK cells in blood or a component thereof, lymphocytes can be contacted in the typically resting state they were in when present in the collected blood in vivo immediately before collection. In some embodiments, the T cells and/or NK cells consist of between 95 and 100% resting cells (Ki-67⁻). In some embodiments, the T cell and/or NK cells that are contacted by replication incompetent recombinant retroviral particles include between 90, 91, 92, 93, 94, and 95% resting cells on the low end of the range and 96, 97, 98, 99, or 100% resting cells on the high end of the range. In some embodiments, the T cells and/or NK cells include naïve cells. In some illustrative embodiments, the subembodiments in this paragraph are included in composition and method aspects for transducing lymphocytes in whole blood.

In illustrative embodiments of aspects herein that include replication incompetent recombinant retroviral particles, contact between the T cells and/or NK cells and the replication incompetent recombinant retroviral particles can facilitate transduction of the T cells and/or NK cells by the replication incompetent recombinant retroviral particles. Not to be limited by theory, during the period of contact, the replication incompetent recombinant retroviral particles identify and bind to T cells and/or NK cells and the T cells and NK cells are “modified” as the term is used herein. At this point the retroviral and host cell membranes start to fuse, and any retroviral pseudotyping elements and/or T cell activation elements, including anti-CD3 antibodies, become integrated into the surface of the modified T cells and/or NK cells. Then, as a next step in the process of transduction, genetic material from the replication incompetent recombinant retroviral particles enters the T cells and/or NK cells at which time the T cells and/or NK cells are “genetically modified” as the phrase is used herein. It is noteworthy that such process might occur hours or even days after the contacting is initiated, and even after non-associated retroviral particles are rinsed away. Then the genetic material is typically integrated into the genomic DNA of the T cells and/or NK cells, at which time the T cells and/or NK cells are now “transduced” as the term is used herein. Similarly, cells can be modified, genetically modified, and/or transduced by recombinant vectors other than replication incompetent recombinant retroviral particles. Cells may also internalize and integrate genetic material into the genomic DNA of the T cells and/or NK cells after transfection, at which time the T cells and/or NK cells are now “stably transfected” as the term is used herein. Accordingly, in illustrative embodiments, any method for modifying and/or genetically modifying lymphocytes (e.g., T cells and/or NK cells) herein, is a method for transducing lymphocytes (e.g., T cells and/or NK cells). It is believed that by day 6 in vivo or ex vivo, after contacting is initiated, the vast majority of modified and genetically modified cells have been transduced. Methods of lentiviral transduction are known. Exemplary methods are described in, e.g., Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505. Throughout this disclosure, a transduced, or in some embodiments a stably transfected, T cell and/or NK cell includes progeny of ex vivo transduced cells that retain at least some of the nucleic acids or polynucleotides that are incorporated into the genome of a cell during the ex vivo transduction. In methods herein that recite “reintroducing” a transduced cell, it will be understood that such cell is typically not in a transduced state when it is collected from the blood of a subject.

Although in illustrative embodiments, T cells and/or NK cells are not activated prior to being contacted with a recombinant retrovirus in methods herein, a T cell activation element in illustrative embodiments is present in the reaction mixture where initial contacting of a recombinant retrovirus and lymphocytes occurs. For example, such T cell activation element can be in solution in the reaction mixture. For example, soluble anti-CD3 antibodies can be present in the reaction mixture during the contacting and optional incubation thereafter, at 25-200, 50-150, 75-125, or 100 ng/ml. In illustrative embodiments, the soluble anti-CD3 antibodies are multivalent such as bivalent, tetravalent, or a higher order valency. In illustrative embodiments, the T cell activation element is associated with the retroviral surface. The T cell activation element can be any T cell activation element provided herein. In illustrative embodiments, the T cell activation element can be anti-CD3, such as anti-CD3 scFv, or anti-CD3 scFvFc. Accordingly, in some embodiments, the replication incompetent recombinant retroviral particle can further include a T cell activation element, which in further illustrative examples is associated with the external side of the surface of the retrovirus.

The contacting step of a method for transducing and/or a method for modifying or genetically modifying lymphocytes in whole blood, provided herein, typically includes an initial step in which the retroviral particle, typically a population of retroviral particles, are brought into contact with blood cells, typically a population of blood cells that includes an anticoagulant and/or additional blood components other than PBMCs, that are not present after a PBMC enrichment procedure, while in suspension in a liquid buffer and/or media to form a transduction reaction mixture. This contacting, as in other aspects provided herein, can be followed by an optional incubating period in this reaction mixture that includes the retroviral particles and the blood cells comprising lymphocytes (e.g., T cells and/or NK cells) in suspension. In methods for modifying T cells and/or NK cells in blood or a component thereof, the reaction mixture can include at least one, two, three, four, five, or all additional blood components as disclosed herein, and in illustrative embodiments includes one or more anticoagulants.

The transduction reaction mixture in any of the aspects provided herein can be incubated at between 23 and 39° C., and in some illustrative embodiments at 37° C., in an optional incubation step after the initial contacting of retroviral particles and lymphocytes. In certain embodiments, the transduction reaction can be carried out at 37-39° C. for faster fusion/transduction. In some embodiments, the contacting step is a cold contacting step as discussed elsewhere herein, with an optional incubating step. In some embodiments, the cold contacting step is performed at temperatures less than 37° C., such as between 1° C. and 25° C. or 2° C. and 6° C. The optional incubating associated with the contacting step at these temperatures can be performed for any length of time discussed herein, for example in the Exemplary Embodiments section. In illustrative embodiments, the optional incubating associated with these temperatures is performed for 8 hours, 6 hours, 4 hours, 2 hours, and in illustrative embodiments 1 hour or less.

In some embodiments, including illustrative embodiments where contacting is performed on a filter, the contacting is carried out at a lower temperature, for example between 2° C. and 25° C., referred to herein as cold contacting, and then retroviral particles that remain unassociated in suspension are removed from the reaction mixture, for example by washing the reaction mixture over a filter, such as a leukoreduction filter, that retains leukocytes including T cells and NK cells, but not free, unassociated viral particles. The cells and retroviral particles when brought into contact in the transduction reaction mixture can be immediately processed to remove the retroviral particles that remain free in suspension and not associated with cells, from the cells. Optionally, the cells in suspension and retroviral particles whether free in suspension or associated with the cells in suspension, can be incubated for various lengths of time, as provided herein for a contacting step in a method provided herein. Before further steps, a wash can be performed, regardless of whether such cells will be studied in vitro, ex vivo or introduced into a subject. Such suspension can include allowing cells and retroviral particles to settle or causing such settling through application of a force, such as a centrifugal force, to the bottom of a vessel or chamber, as discussed in further detail herein. In illustrative embodiments, such g force is lower than the g forces used successfully in spinoculation procedures. Further contacting times and discussions regarding contacting and the optional incubation, are discussed further herein, for example in the Exemplary Embodiments section.

Current methods require extensive periods of ex vivo expansion of genetically modified lymphocytes before formulation and reintroduction into a subject. There is a long-felt need for effective point-of-care adoptive cellular therapy that would allow a subject to have blood drawn (collected), lymphocytes modified and reintroduced in a single visit. The methods provided herein allow for rapid ex vivo processing of lymphocytes, and in certain illustrative embodiments, PBMCs, and in other illustrative embodiments, total nucleated cells (TNCs), without an ex vivo expansion step, fundamentally simplifying the delivery of adoptive cell therapies, for example by providing such point-of-care methods, and in some illustrative embodiments, in shorter periods of time (rapid point-of-care (rPOC)). Illustrative methods are disclosed herein for modifying lymphocytes, especially NK cells and in illustrative embodiments, T cells, that are much shorter and simpler than prior methods. Accordingly, in some embodiments, the contacting step in any method provided herein of transducing, genetically modifying, and/or modifying a PBMC or a lymphocyte, typically a T cell and/or an NK cell, can be performed (or can occur) for any of the time periods provided in this specification, included, but not limited to those provided in the Exemplary Embodiments section. For example, said contacting can be for less than 24 hours, for example, less than 12 hours, less than 8 hours, less than 4 hours, less than 2 hours, less than 1 hour, less than 30 minutes or less than 15 minutes, but in each case there is at least an initial contacting step in which retroviral particles and cells come into contact in suspension in a transduction reaction mixture before retroviral particles that remain in suspension not associated with a cell, are separated from cells and typically discarded, as discussed in further detail herein. It should be noted, but not intending to be limited by theory, that it is believed that contacting begins at the time that retroviral particles and lymphocytes are combined together, typically by adding a solution containing the retroviral particles into a solution containing lymphocytes (e.g., T cells and/or NK cells).

After initial contacting, including initial cold contacting, in some embodiments there is an incubating of the reaction mixture containing cells and recombinant nucleic acid vectors, which in illustrative embodiments are retroviral particles, in suspension for a specified time period without removing recombinant nucleic acid vectors (e.g., retroviral particles) that remain free in solution and not associated with cells. This incubating is sometimes referred to herein as an optional incubation. Thus, in illustrative embodiments, the contacting (including initial contacting and optional incubation) can be performed (or can occur) for between 15 minutes and 12 hours, between 15 minutes and 10 hours, or between 15 minutes and 8 hours, or any of the times included in the Exemplary Embodiments section. In certain embodiments that comprise a cold contacting step, a secondary incubation is performed by suspending cells after an optional wash step such that recombinant nucleic acid vectors, and in illustrative embodiments retroviral particles, that are not associated with a cell are washed away. In illustrative embodiments, the secondary incubation is performed at temperatures between 32° C. and 42° C., such as at 37° C. The optional secondary incubation can be performed for any length of time discussed herein. In illustrative embodiments, the optional secondary incubation is performed for 6 hours or less. Thus, in illustrative embodiments, the contacting (including initial contacting and optional incubation) can be performed (or can occur) (where as indicated in general herein the low end of a selected range is less than the high end of the selected range) for between 30 seconds or 1, 2, 5, 10, 15, 30, or 45 minutes, or 1, 2, 3, 4, 5, 6, 7, or 8 hours on the low end of the range, and between 10 minutes, 15 minutes, 30 minutes, or 1, 2, 4, 6, 8, 10, 12, 18, 24, 36, 48, and 72 hours on the high end of the range. Thus, in some embodiments, after the time when a reaction mixture is formed by adding retroviral particles to lymphocytes, the reaction mixture can be incubated for between 5 minutes on the low end of the range and 10, 15, or 30 minutes or 1, 2, 3, 4, 5, 6, 8, 10 or 12 hours on the high end of the range. In other embodiments, the reaction mixture can be incubated for between 15 minutes and 12 hours, 15 minutes and 10 hours, 15 minutes and 8 hours, 15 minutes and 6 hours, 15 minutes and 4 hours, 15 minutes and 2 hours, 15 minutes and 1 hour, 15 minutes and 45 minutes, or 15 minutes and 30 minutes. In other embodiments, the reaction mixture can be incubated for between 30 minutes and 12 hours, 30 minutes and 10 hours, 30 minutes and 8 hours, 30 minutes and 6 hours, 30 minutes and 4 hours, 30 minutes and 2 hours, 30 minutes and 1 hour, or 30 minutes and 45 minutes. In other embodiments, the reaction mixture can be incubated for between 1 hour and 12 hours, 1 hour and 8 hours, 1 hour and 4 hours, or 1 hour and 2 hours. In another illustrative embodiment, the contacting is performed for between an initial contacting step only (without any further incubating in the reaction mixture including the retroviral particles free in suspension and cells in suspension) without any further incubation in the reaction mixture, or a 5 minute, 10 minute, 15 minute, 30 minute, or 1 hour incubation in the reaction mixture.

After the indicated time period for the initial contacting and optional incubation that can be part of the contacting step, blood cells or a T cell and/or NK cell-containing fraction thereof in the reaction mixture, are separated from retroviral particles that are not associated with such cells. For example, this can be performed using a PBMC enrichment procedure (e.g., a Ficoll gradient in a Sepax unit), or in certain illustrative embodiments provided herein, by filtering the reaction mixture over a leukocyte depletion filter set assembly, and then collecting the leukocytes, which include T cells and NK cells. In another embodiment, this can be performed by centrifugation of the reaction mixture at a relative centrifugal force less than 500 g, for example 400 g, or between 300 and 490 g, or 350 and 450 g. Such centrifugation to separate retroviral particles from cells can be performed for example, for between 5 minutes and 15 minutes, or between 5 minutes and 10 minutes. In illustrative embodiments where centrifugal force is used to separate cells from retroviral particles that are not associated with cells, such g force is typically lower than the g forces used successfully in spinoculation procedures.

In some illustrative embodiments, a method provided herein in any aspect, does not involve performing a spinoculation. In such embodiments, the cell or cells are not subjected to a spinoculation of at least 400 g, 500 g, 600 g, 700 g, or 800 g for at least 15 minutes. In some embodiments, the cell or cells are not subjected to a spinoculation of at least 800 g for at least 10, 15, 20, 25, 30, 35, 40, or 45 minutes. In some embodiments, spinoculation is included as part of a contacting step. In illustrative embodiments, when spinoculation is performed there is no additional incubating as part of the contacting, as the time of the spinoculation provides the incubation time of the optional incubation discussed above. In other embodiments, there is an additional incubation after the spinoculating of between 15 minutes and 4 hours, 15 minutes and 2 hours, or 15 minutes and 1 hour. The spinoculation can be performed for example, for 30 minutes to 120 minutes, typically for at least 60 minutes, for example for 60 minutes to 180 minutes, or 60 minutes to 90 minutes. The spinoculation is typically performed in a centrifuge with a relative centrifugal force of at least 800 g, and more typically at least 1200 g, for example between 800 g and 2400 g, 800 g and 1800 g, 1200 g and 2400 g, or 1200 g and 1800 g. After the spinoculation, such methods typically involve an additional step of resuspending the pelleted cells and retroviral particles, and then removing retroviral particles that are not associated with cells according to steps discussed above when spinoculation is not performed.

The contacting step including the optional incubation therein, and the spinoculation, in embodiments that include spinoculation, can be performed at between 4° C. and 42° C. or 20° C. and 37° C. In certain illustrative embodiments, spinoculation is not performed and the contacting and associated optional incubation are carried out at 20-25° C. for 4 hours or less, 2 hours or less, 1 hour or less, 30 minutes or less, 15 minutes or less, or 15 minutes to 2 hours, 15 minutes to 1 hour, or 15 minutes to 30 minutes.

Methods of genetically modifying lymphocytes provided according to any method herein, typically include insertion into the cell of a polynucleotide comprising one or more transcriptional units encoding any transgene, for example a CAR or a lymphoproliferative element, or in illustrative embodiments encoding both a CAR and a lymphoproliferative element according to any of the CAR and lymphoproliferative element embodiments provided herein. Such CAR and lymphoproliferative elements can be provided to support the shorter and more simplified methods provided herein, which can support expansion of modified, genetically modified, and/or transduced T cells and/or NK cells after the contacting and optional incubation. Accordingly, in exemplary embodiments of any methods provided herein, lymphoproliferative elements can be delivered from the genome of the retroviral particles inside genetically modified, and/or transduced T cells and/or NK cells, such that those cells have the characteristics of increased proliferation and/or survival disclosed in the Lymphoproliferative Elements section herein. In exemplary embodiments of any methods provided herein, the genetically modified T cell or NK cell is capable of engraftment in vivo in mice and/or enrichment in vivo in mice for at least 7, 14, or 28 days. A skilled artisan will recognize that such mice may be treated or otherwise genetically modified so that any immunological differences between the genetically modified T cell and/or NK cell do not result in an immune response being elicited in the mice against any component of the lymphocyte transduced by the replication incompetent recombinant retroviral particle.

Media that can be included in a contacting step, for example when the cells and retroviral particles are initially brought into contact, or in any aspects provided herein, during optional incubation periods with the reaction mixture thereafter that include retroviral particles and cells in suspension in the media, or media that can be used during cell culturing and/or during various wash steps in any aspects provided herein, can include base media such as commercially available media for ex vivo T cell and/or NK cell culture. Non-limiting examples of such media include, X-VIVO™ 15 Chemically Defined, Serum-free Hematopoietic Cell Medium (Lonza) (2018 catalog numbers BE02-060F, BE02-00Q, BE-02-061Q, 04-744Q, or 04-418Q), ImmunoCult™-XF T Cell Expansion Medium (STEMCELL Technologies) (2018 catalog number 10981), PRIME-XV® T Cell Expansion XSFM (Irvine Scientific) (2018 catalog number 91141), AIM V® Medium CTS™ (Therapeutic Grade) (Thermo Fisher Scientific (Referred to herein as “Thermo Fisher”), or CTS™ Optimizer™ media (Thermo Fisher) (2018 catalog numbers A10221-01 (basal media (bottle)), and A10484-02 (supplement), A10221-03 (basal media (bag)), A1048501 (basal media and supplement kit (bottle)) and, A1048503 (basal media and supplement kit (bag)). Such media can be a chemically defined, serum-free formulation manufactured in compliance with cGMP, as discussed herein for kit components. The media can be xeno-free and complete. In some embodiments, the base media has been cleared by regulatory agencies for use in ex vivo cell processing, such as an FDA 510(k) cleared device. In some embodiments, the media is the basal media with or without the supplied T cell expansion supplement of 2018 catalog number A1048501 (CTS™ OpTmizer™ T Cell Expansion SFM, bottle format) or A1048503 (CTS™ OpTmizer™ T Cell Expansion SFM, bag format) both available from Thermo Fisher (Waltham, Mass.). Additives such as human serum albumin, human AB+ serum, and/or serum derived from the subject can be added to the transduction reaction mixture. Supportive cytokines can be added to the transduction reaction mixture, such as IL2, IL7, or IL15, or those found in human sera. dGTP can be added to the transduction reaction in certain embodiments.

In some embodiments of any method herein that includes a step of modifying lymphocytes (e.g., T cells and/or NK cells), the cells can be contacted with a retroviral particle without prior activation. In some embodiments of any method herein that includes a step of genetically modifying T cells and/or NK cells, the T cells and/or NK cells have not been incubated on a substrate that adheres to monocytes for more than 4 hours in one embodiment, or for more than 6 hours in another embodiment, or for more than 8 hours in another embodiment before the transduction. In one illustrative embodiment, the T cells and/or NK cells have been incubated overnight on an adherent substrate to remove monocytes before the transduction. In another embodiment, the method can include incubating the T cells and/or NK cells on an adherent substrate that binds monocytes for no more than 30 minutes, 1 hour, or 2 hours before the transduction. In another embodiment, the T cells and/or NK cells are exposed to no step of removing monocytes by an incubation on an adherent substrate before said transduction step. In another embodiment, the T cells and/or NK cells are not incubated with or exposed to a bovine serum, such as a cell culturing bovine serum, for example fetal bovine serum before or during a contacting step and/or a modifying and/or a genetically modifying and/or transduction step.

Some or all of the steps of the methods for modifying provided herein, or uses of such methods, are performed in a closed system. Thus, reaction mixtures formed in such methods, and modified, genetically modified, and/or transduced lymphocytes (e.g., T cells and/or NK cells) made by such methods, can be contained within such a closed system. A closed system is a cell processing system that is generally closed or fully closed to an environment, such as an environment within a room or even the environment within a hood, outside of the conduits such as tubes, and chambers, of the system in which cells are processed and/or transported. One of the greatest risks to safety and regulatory control in the cell processing procedure is the risk of contamination through frequent exposure to the environment as is found in traditional open cell culture systems. To mitigate this risk, particularly in the absence of antibiotics, some commercial processes have been developed that focus on the use of disposable (single-use) equipment. However, even with their use under aseptic conditions, there is always a risk of contamination from the opening of flasks to sample or add additional growth media. To overcome this problem, methods provided herein, which are typically ex vivo methods, are typically performed within a closed-system. Such a process is designed and can be operated such that the product is not exposed to the outside environment. Material transfer occurs via sterile connections, such as sterile tubing and sterile welded connections. Air for gas exchange can occur via a gas permeable membrane, via 0.2 μm filter to prevent environmental exposure. In some illustrative embodiments, the methods are performed on T cells, for example to provide modified and in illustrative embodiments genetically modified T cells.

Such closed system methods can be performed with commercially available devices. Different closed system devices can be used at different steps within a method and the cells can be transferred between these devices using tubing and connections such as welded, luer, spike, or clave ports to prevent exposure of the cells or media to the environment. For example, blood can be collected into an IV bag or syringe, optionally including an anticoagulant, and in some aspects, transferred to a Sepax 2 device (Biosafe) for PBMC enrichment and isolation. In other embodiments, whole blood can be filtered to collect leukocytes using a leukoreduction filter assembly. The isolated PBMCs or isolated leukocytes can be transferred to a chamber of a G-Rex device for an optional activation, a transduction and optional expansion. Alternatively, collected blood can be transduced in a blood bag, for example, the bag in which it was collected. Finally, the cells can be harvested and collected into another bag using a Sepax 2 device. The methods can be carried out in any device or combination of devices adapted for closed system T cell and/or NK cell production. Non-limiting examples of such devices include G-Rex devices (Wilson Wolf), GatheRex (Wilson Wolf), Sepax 2 (Biosafe), WAVE Bioreactors (General Electric), a CultiLife Cell Culture bag (Takara), a PermaLife bag (OriGen), CliniMACS Prodigy (Miltenyi Biotec), and VueLife bags (Saint-Gobain). In illustrative embodiments, the optional activating, the transducing and optional expanding can be performed in the same chamber or vessel in the closed system. For example, in illustrative embodiments, the chamber can be a chamber of a G-Rex device and PBMCs or leukocytes can be transferred to the chamber of the G-Rex device after they are enriched and isolated, and can remain in the same chamber of the G-Rex device until harvesting.

Methods provided herein can include transferring blood and cells therein and/or fractions thereof, as well as lymphocytes before or after they are contacted with retroviral particles, between vessels within a closed system, which thus is without environmental exposure. Vessels used in the closed system, for example, can be a tube, bag, syringe, or other container. In some embodiments, the vessel is a vessel that is used in a research facility. In some embodiments, the vessel is a vessel used in commercial production. In other embodiments, the vessel can be a collection vessel used in a blood collection process. Methods for modifying herein, typically involve a contacting step wherein lymphocytes are contacted with a replication incompetent recombinant retroviral particle. The contacting in some embodiments, can be performed in the vessel, for example, within a blood bag. Blood and various lymphocyte-containing fractions thereof, can be transferred from the vessel to another vessel (for example from a first vessel to a second vessel) within the closed system for the contacting. The second vessel can be a cell processing compartment of a closed device, such as a G-Rex device. In some embodiments, after the contacting the modified and in illustrative embodiments genetically modified (e.g., transduced) cells can be transferred to a different vessel within the closed system (i.e., without exposure to the environment). Either before or after this transfer the cells are typically washed within the closed system to remove substantially all or all of the retroviral particles. In some embodiments, a process disclosed herein, from collection of blood, to contacting (e.g., transduction), optional incubating, and post-incubation isolation and optional washing, is performed for between 15 minutes, 30 minutes, or 1, 2, 3, or 4 hours on the low end of the range, and 4, 8, 10, or 12 hours on the high end of the range.

Various embodiments of this method, as well as other aspects, such as use of NK cells and T cells made by such a method, are disclosed in detail herein. Furthermore, various elements or steps of such method aspects for transducing, genetically modifying, and/or modifying a PBMC, lymphocyte, T cell and/or NK cell, are provided herein, for example in this section and the Exemplary Embodiments section, and such methods include embodiments that are provided throughout this specification, as further discussed herein, For example, embodiments of any of the aspects for transducing, genetically modifying, and/or modifying a PBMC or a lymphocyte, for example an NK cell or in illustrative embodiments, a T cell, provided for example in this section and in the Exemplary Embodiments section, can include any of the embodiments of replication incompetent recombinant retroviral particles provided herein, including those that include one or more lymphoproliferative element, CAR, pseudotyping element, control element, activation element, membrane-bound cytokine, miRNA, Kozak-type sequence, WPRE element, triple stop codon, and/or other element disclosed herein, and can be combined with methods herein for producing retroviral particles using a packaging cell. In certain illustrative embodiments, the retroviral particle is a lentiviral particle. Such a method for modifying, genetically modifying, and/or transducing a PBMC or a lymphocyte, such as a T cell and/or NK cell can be performed in vitro or ex vivo. A skilled artisan will recognize that details provided herein for transducing, genetically modifying, and/or modifying PBMCs or lymphocytes, such as T cell(s) and/or NK cell(s) can apply to any aspect that includes such step(s).

Introduction or reintroduction, also referred to herein as administration and readministration, of modified and in illustrative embodiments genetically modified lymphocytes, or in some embodiments, replication incompetent retroviral particles (“RIPs”), into a subject in methods provided herein can be via any route known in the art. Such introduction or reintroduction of genetically modified lymphocytes typically involves suspending i) modified and/or ii) genetically modified and/or iiia) transduced or iiib) transfected cells, in a delivery solution to form a cell formulation that can be introduced or reintroduced into a subject as discussed in further detail herein. Such introduction of RIPs for example, can involve suspension of the RIPs in a delivery solution to form a transducing formulation that can be introduced into a subject. For example, introduction or RIPS, lymphocytes or modified lymphocytes, or reintroduction for lymphocytes or modified lymphocytes, can be delivery via infusion into a blood vessel of the subject. In some embodiments, RIPS or modified lymphocytes (e.g., T cells and/or NK cells) are administered or otherwise introduced, or reintroduced back, for lymphocytes or modified lymphocytes, into a subject by intraperitoneal administration, intratumoral administration, intramuscular administration, or in illustrative embodiments by subcutaneous administration.

Some administered cells are modified with a nucleic acid encoding a lymphoproliferative element. Not to be limited by theory, in non-limiting illustrative methods, the delivery of a polynucleotide encoding a lymphoproliferative element, to a resting T cell and/or NK cell ex vivo, which can integrate into the genome of the T cell or NK cell, provides that cell with a driver for in vivo expansion without the need for lymphodepleting the host. Thus, in illustrative embodiments, the subject is not exposed to a lymphodepleting agent within 1, 2, 3, 4, 5, 6, 7, 10, 14, 21, or 28 days, or within 1 month, 2 months, 3 months or 6 months of performing the contacting, during the contacting, and/or within 1, 2, 3, 4, 5, 6, 7, 10, 14, 21, or 28 days, or within 1 month, 2 months, 3 months or 6 months after the modified T cells and/or NK cells are reintroduced back into the subject. Furthermore, in non-limiting illustrative embodiments, methods provided herein can be performed without exposing the subject to a lymphodepleting agent during a step wherein a replication incompetent recombinant retroviral particle is in contact with resting T cells and/or resting NK cells of the subject and/or during the entire ex vivo method. Hence, methods of expanding modified and in illustrative embodiments genetically modified T cells and/or NK cells in a subject in vivo is a feature of some embodiments of the present disclosure. In illustrative embodiments, such methods are ex vivo propagation-free or substantially propagation-free.

This entire method/process from blood draw from a subject to reintroduction of modified and in illustrative embodiments genetically modified lymphocytes into the subject after ex vivo transduction of T cells and/or NK cells, in non-limiting illustrative embodiments of any aspects provided herein, can occur over a time period less than 48 hours, less than 36 hours, less than 24 hours, less than 12 hours, less than 11 hours, less than 10 hours, less than 9 hours, less than 8 hours, less than 7 hours, less than 6 hours, less than 5 hours, less than 4 hours, less than 3 hours, 2 hours, or less than 2 hours. In any of the embodiments disclosed herein, introduction or reintroduction of the modified lymphocytes can be performed by intravenous injection, intraperitoneal administration, subcutaneous administration, intratumoral, or intramuscular administration. In other embodiments, the entire method/process from blood draw/collection from a subject to reintroduction of modified lymphocytes into the subject after ex vivo transduction of T cells and/or NK cells, in non-limiting illustrative embodiments herein, occurs over a time period between 1 hour and 12 hours, 2 hours and 8 hours, 1 hour and 3 hours, 2 hours and 4 hours, 2 hours and 6 hours, 4 hours and 12 hours, 4 hours and 24 hours, 8 hours and 24 hours, 8 hours and 36 hours, 8 hours and 48 hours, 12 hours and 24 hours, 12 hours and 36 hours, or 12 hours and 48 hours, or over a time period between 15, 30, 60, 90, 120, 180, and 240 minutes on the low end of the range, and 120, 180, and 240, 300, 360, 420, and 480 minutes on the high end of the range. In other embodiments, the entire method/process from blood draw/collection from a subject to reintroduction of modified and in illustrative embodiments genetically modified lymphocytes into the subject after ex vivo transduction of T cells and/or NK cells, occurs over a time period between 1, 2, 3, 4, 6, 8, 10, and 12 hours on the low end of the range, and 8, 9, 10, 11, 12, 14, 18, 24, 36, or 48 hours on the high end of the range. In some embodiments, the modified and genetically modified T cells and/or NK cells are separated from the non-associated replication incompetent recombinant retroviral particles after the time period in which contact occurs.

Because methods provided herein for modifying lymphocytes, and associated methods for performing adoptive cell therapy can be performed in significantly less time than prior methods, fundamental improvements in patient care and safety as well as product manufacturability are made possible. Therefore, such processes are expected to be favorable in the view of regulatory agencies responsible for approving such processes when carried out in vivo for therapeutic purposes. For example, the subject in non-limiting examples of any aspects provided herein that include a subject, can remain in the same building (e.g., infusion clinic) or room as the instrument processing their blood or sample for the entire time that the sample is being processed before modified T cells and/or NK cells are reintroduced into the patient. In non-limiting illustrative embodiments, a subject remains within line of site and/or within 100, 50, 25, or 12 feet or arm's distance of their blood or cells that are being processed, for the entire method/process from blood draw/collection from the subject to reintroduction of blood to the subject after ex vivo transduction of T cells and/or NK cells. In other non-limiting illustrative embodiments, a subject remains awake and/or at least one person can continue to monitor the blood or cells of the subject that are being processed, throughout and/or continuously for the entire method/process from blood draw/collection from the subject to reintroduction of blood to the subject after ex vivo transduction of T cells and/or NK cells. Because of improvements provided herein, the entire method/process for adoptive cell therapy and/or for transducing resting T cells and/or NK cells from blood draw/collection from the subject to reintroduction of blood to the subject after ex vivo transduction of T cells and/or NK cells can be performed with continuous monitoring by a human In other non-limiting illustrative embodiments, at no point the entire method/process from blood draw/collection from the subject to reintroduction of blood to the subject after ex vivo transduction of T cells and/or NK cells, are blood cells incubated in a room that does not have a person present. In other non-limiting illustrative embodiments, the entire method/process from blood draw/collection from the subject to reintroduction of blood to the subject after ex vivo transduction of T cells and/or NK cells, is performed next to the subject and/or in the same room as the subject and/or next to the bed or chair of the subject. Thus, sample identity mix-ups can be avoided, as well as long and expensive incubations over periods of days or weeks. This is further provided by the fact that methods provided herein are readily adaptable to closed and automated blood processing systems, where a blood sample and its components that will be reintroduced into the subject, only make contact with disposable, single-use components.

Methods for modifying, genetically modifying, and/or transducing lymphocytes such as T cells and/or NK cells provided herein, can be part of a method for performing adoptive cell therapy. Typically, methods for performing adoptive cell therapy include steps of collecting blood from a subject, and returning modified, genetically modified, and/or transduced lymphocytes (e.g., T cells and/or NK cells) to the subject. The present disclosure provides various treatment methods using a CAR. A CAR of the present disclosure, when present in a T lymphocyte or an NK cell, can mediate cytotoxicity toward a target cell. A CAR of the present disclosure binds to an antigen present on a target cell, thereby mediating killing of a target cell by a T lymphocyte or an NK cell genetically modified to produce the CAR. The ASTR of the CAR binds to an antigen present on the surface of a target cell. The present disclosure provides methods of killing, or inhibiting the growth of, a target cell, the method involving contacting a cytotoxic immune effector cell (e.g., a cytotoxic T cell, or an NK cell) that is genetically modified to produce a subject CAR, such that the T lymphocyte or NK cell recognizes an antigen present on the surface of a target cell, and mediates killing of the target cell. The target cell can be a cancer cell, for example, and autologous cell therapy methods herein, can be methods for treating cancer, in some illustrative embodiments. In these embodiments, the subject can be a an animal or human suspected of having cancer, or more typically, a subject that is known to have cancer. In some embodiments for treating a PDL-1 positive cancer, and in illustrative embodiments a PDL-1 positive diffuse large B cell lymphoma (DLBCL), genetically modified cells can be administered in combination with an anti-PDL-1 antibody or antibody mimetic.

In some illustrative embodiments, cells are introduced or reintroduced into the subject by infusion into a vein or artery, especially when neutrophils are not present in a preparation of lymphocytes that have been contacted with retroviral particles and are ready to be reintroduced, or by subcutaneous, intratumoral, or intramuscular administration, for embodiments where at least 1%, 2%, 3%, 4%, 5%, 7.5%, 10%, 15%, 20% or 25% of the cells, or between 1% and 90%, 1% and 75%, 1% and 50%, 1% and 25%, 1% and 20%, 1% and 10%, 5% and 90%, 5% and 75%, 5% and 50%, 5% and 25%, 5% and 20%, 5% and 10%, 10% and 90%, 10% and 75%, 10% and 50%, 10% and 25%, or 10% and 20%, of the cells in a cell formulation to be administered are neutrophils. Such embodiments, can include coadministration or sequential administration with hyaluronidase, as discussed in further detail herein. In any of the embodiments disclosed herein, the number of lymphocytes, and in illustrative embodiments modified T cells and/or NK cells, present in cell formulations provided herein and optionally reinfused or in illustrative embodiments, subcutaneously delivered into a subject can be between 1×10³, 2.5×10³, 5×10³, 1×10⁴, 2.5×10⁴, 5×10⁴, 1×10⁵, 2.5×10⁵, 5×10⁵, 1×10⁶, 2.5×10⁶, 5×10⁶, and 1×10⁷ cells/kg on the low end of the range and 5×10⁴, 1×10⁵, 2.5×10⁵, 5×10⁵, 1×10⁶, 2.5×10⁶, 5×10⁶, 1×10⁷, 2.5×10⁷, 5×10⁷, 1×10⁸, 1×10⁹, and 1×10¹⁰ cells/kg on the high end of the range. In certain embodiments, the number of lymphocytes, and in illustrative embodiments modified T cells and/or NK cells, present in cell formulations herein and optionally reinfused or otherwise delivered into a subject can be between 1×10⁴, 2.5×10⁴, 5×10⁴, and 1×10⁵ cells/kg on the low end of the range and 2.5×10⁴, 5×10⁴, 1×10⁵, 2.5×10⁵, 5×10⁵, 1×10⁶, 1×10⁷, 2.5×10⁷, 5×10⁷, and 1×10⁸ cells/kg on the high end of the range, or between 1×10⁴ cells/kg on the low end of the range and 2.5×10⁴, 5×10⁴, 1×10⁵, 2.5×10⁵, 5×10⁵, 1×10⁶, 1×10⁷, 2.5×10⁷, 5×10⁷, and 1×10⁸ cells/kg on the high end of the range. In some embodiments, the number of lymphocytes, and in illustrative embodiments T cells and/or NK cells present in cell formulations herein and optionally reinfused, or delivered intratumorally, intramuscularly, subcutaneously, or otherwise delivered into a subject can be between 5×10⁵, 1×10⁶, 2.5×10⁶, 5×10⁶, 1×10⁷, 2.5×10⁷, 5×10⁷, and 1×10⁸ cells on the low end of the range and 2.5×10⁶, 5×10⁶, 1×10⁷, 2.5×10⁷, 5×10⁷, 1×10⁸, 2.5×10⁸, 5×10⁸, and 1×10⁹ cells on the high end of the range. In some embodiments, the number of lymphocytes, and in illustrative embodiments T cells and/or NK cells, present in cell formulations herein and available for infusion, reinfusion, or other delivery means (e.g., subcutaneous delivery) into a 70 kg subject or patient is between 7×10⁵ and 2.5×10⁸ cells. In other embodiments, the number of lymphocytes, and in illustrative embodiments T cells and/or NK cells present in cell formulations herein and available for transduction is approximately 7×10⁶ plus or minus 10%.

In any of the embodiments and aspects provided herein that include a T cell, NK cell, B cell, or stem cell, the cell can be an autologous cell or an allogeneic cell. In some embodiments, the allogeneic cell can be a genetically engineered allogeneic cell. Allogeneic cells, such as allogeneic T cells, and methods for genetically engineering allogeneic cells, are known in the art. In some embodiments where the allogeneic cell is a T cell, the T cell has been genetically engineered such that at least one component of the TCR complex is functionally impaired and/or is at least partially deleted. In some embodiments, the T cell has been genetically engineered such that the expression of at least one component of the TCR complex has been reduced or eliminated. In some embodiments, the allogeneic cell can be modified such that it is missing all or part of the B2 microglobulin gene. In some embodiment, allogeneic cells can include any of the lymphoproliferative elements and/or CLEs disclosed herein. The use of lymphoproliferative elements and CLEs can reduce the required number of cells and can facilitate cell manufacturing of T cells, NK cells, B cells, or stem cells. In some embodiments, the allogeneic cell can be an immortalized cell. In any of the aspects or embodiments herein that include an allogeneic cell, steps that include collecting blood or contacting a cell with a replication incompetent recombinant retroviral particle can be eliminated. For example, for treating a subject with an allogeneic CAR-T cell, a T cell may have been previously genetically modified, and the genetically modified allogeneic CAR-T cell is administered to the subject without collecting blood from the subject. In some embodiments, the allogeneic cell is administered subcutaneously. In some embodiments, the allogeneic cell is administered intravenously. In some embodiments, the allogeneic cell is administered intraperitoneally.

In some embodiments of any of the methods provided herein for modifying lymphocytes (e.g., T cells and/or NK cells), and aspects directed to use of replication incompetent recombinant retroviral particles (RIPs) in the manufacture of a kit for modifying T cells and/or NK cells of a subject, the modified, genetically modified, and/or transduced lymphocyte (e.g., T cell and/or NK cell) or population thereof, or the RIPs in compositions provided herein without cells, such as GMP RIP compositions, are introduced or reintroduced into the subject. Introduction or reintroduction of the modified and in illustrative embodiments genetically modified lymphocytes into a subject can be via any route known in the art. For example, introduction or reintroduction can be delivery via infusion into a blood vessel of the subject. Intratumor, intraperitoneal, intramuscular, and in certain illustrative embodiments, subcutaneous. In some embodiments, the modified, genetically modified, and/or transduced lymphocyte (e.g., T cell and/or NK cell) or population thereof, undergo 4 or fewer cell divisions ex vivo prior to being introduced or reintroduced into the subject. In some embodiments, the lymphocyte(s) used in such a method are resting T cells and/or resting NK cells that are in contact with the replication incompetent recombinant retroviral particles for between 1 hour and 12 hours. In some embodiments, no more than 12 hours, 10 hours, 8 hours, 6 hours, 4 hours, 2 hours, or 1 hour pass(es) between the time blood is collected from the subject and the time the modified and/or genetically modified T cells and/or NK cells are formulated for delivery and/or are reintroduced into the subject. In some embodiments, all steps after the blood are collected and before the blood is reintroduced, are performed in a closed system in which a person monitors the closed system throughout the processing.

In some embodiments of the methods and compositions disclosed herein, the modified and in illustrative embodiments genetically modified T cells and/or NK cells are introduced back, reintroduced, reinfused or otherwise delivered into the subject without additional ex vivo manipulation, such as stimulation and/or activation of T cells and/or NKs. In the prior art methods, ex vivo manipulation is used for stimulation/activation of T cells and/or NK cells and for expansion of genetically modified T cells and/or NK cells prior to introducing the genetically modified T cells and/or NK cells into the subject. In prior art methods, this generally takes days or weeks and requires a subject to return to a clinic for a blood infusion days or weeks after an initial blood draw. In some embodiments of the methods and compositions disclosed herein, T cells and/or NK cells are not stimulated ex vivo by exposure to anti-CD3 alone or anti-CD3 in combination with co-stimulation by, for example, anti-CD28, either in solution or attached to a solid support such as, for example, beads coated with anti-CD3/anti-CD28, prior to contacting the T cells and/or NK cells with the replication incompetent recombinant retroviral particles. As such provided herein is an ex vivo propagation-free method. In other embodiments, modified and in illustrative embodiments genetically modified T cells and/or NK cells are not expanded ex vivo, or only expanded for a small number of cell divisions (e.g., 1, 2, 3, 4, or 5 rounds of cell division), but are rather expanded, or predominantly expanded, in vivo, i.e., within the subject. In some embodiments, no additional media is added to allow for further expansion of the cells. In some embodiments, no cell manufacturing of the primary blood lymphocytes (PBLs) occurs while the PBLs are contacted with the replication incompetent recombinant retroviral particles. In illustrative embodiments, no cell manufacturing of the PBLs occurs while the PBLs are ex vivo. In traditional methods of adoptive cell therapy, subjects are lymphodepleted prior to reinfusion with genetically modified T cells and or NK cells. In some embodiments, patients or subjects are not lymphodepleted prior to infusion or reinfusion with modified and/or genetically modified T cells and or NK cells. However, embodiments of the methods and compositions disclosed herein can be used on pre-activated or pre-stimulated T cells and/or NK cells as well. In some embodiments, T cells and/or NK cells can be stimulated ex vivo by exposure to anti-CD3 with or without anti-CD28 solid supports prior to contacting the T cells and/or NK cells with the replication incompetent recombinant retroviral particles. In some embodiments, the T cells and/or NK cells can be exposed to anti-CD3/anti-CD28 solid supports for less than 1, 2, 3, 4, 6, 8, 10, 12, 14, 16, 18, or 24 hours, including no exposure, before the T cells and/or NK cells are contacted the replication incompetent recombinant retroviral particles. In illustrative embodiments, the T cells and/or NK cells can be exposed to anti-CD3/anti-CD28 solid supports for less than 1, 2, 3, 4, 6, or 8 hours before the T cells and/or NK cells are contacted the replication incompetent recombinant retroviral particles.

Enrichment of T and/or NK cells by Positive Selection

In some embodiments, any cell in a cell mixture, cell formulation, or reaction mixture that is useful in adoptive cell therapy, referred to herein as desired cells, such as one or more cell populations of T and/or NK cells, can be enriched prior to formulation for delivery. In some embodiments, the desired cells can be enriched by positive selection prior to being contacted with a recombinant nucleic acid vector, such as a replication incompetent retroviral particle. In other embodiments, the desired cells can be enriched by positive selection after the cell mixture, cell formulation, or reaction mixture is contacted with a recombinant nucleic acid vector, such as a replication incompetent retroviral particle. In some embodiments, enriching the one or more cell populations can be performed at the same time as any of the methods of genetic modification disclosed herein, and in illustrative embodiments genetic modification with a replication incompetent retroviral particle.

Mononuclear cells (such as PBMCs) or TNCs can be isolated from a more complex cell mixture such as whole blood by density-gradient centrifugation or reverse perfusion of a leukoreduction filter assembly, respectively, as described in more detail herein. In some embodiments, the desired cells can have specific cell lineages, such as NK cells, T cells, and/or T cell subsets including naïve, effector, memory, suppressor T-cells, and/or regulatory T cells and can be enriched through the selection of cells expressing one or more surface molecules. In illustrative embodiments, the one or more surface molecules can include CD4, CD8, CD16, CD25, CD27, CD28, CD44, CD45RA, CD45RO, CD56, CD62L, CCR7, KIRs, FoxP3, and/or TCR components such as CD3. Methods using beads conjugated to antibodies directed to one or more surface molecules can be used to enrich for the desired cells using magnetic, density, and size-based separation.

In the process of such antibody-based positive selection methods, binding of the one or more cell surface molecules can lead to signal transduction and alteration of the biology of the bound cell. For example, selection of T cells using beads with attached antibodies to CD3 may lead to CD3 signal transduction and T cell activation. In other examples, binding and signal transduction may lead to further cell differentiation of cells such as naïve or memory T cells. In some embodiments, positive selection is not used to enrich for desired cells such as when it is preferred that the desired cells are not contacted but rather are left untouched.

In some embodiments, the desired cells can be enriched such that the desired cells comprise at least 1%, 2%, 3%, 4%, 5%, 6%, 7%, 8%, 9%, 10%, 15%, 20%, 30%, 40%, 50%, 60%, 70%, 80%, 90%, 95%, 96%, 97%, 98%, or 99% of the cells in a cell mixture, cell formulation, or reaction mixture. In some embodiments, the desired cells can be enriched such that the desired cells comprise between 1%, 2%, 3%, 4%, 5%, 6%, 7%, 8%, 9%, 10%, 20%, 30%, or 40% of the cells in a cell mixture, cell formulation, or reaction mixture on the low end of the range and 10%, 20%, 30%, 40%, 50%, 60%, 70%, 80%, 90%, 95%, 96%, 97%, 98%, or 99% of the cells in a cell mixture, cell formulation, or reaction mixture on the high end of the range. In some embodiments, the desired cells can be enriched such that the desired cells comprise between 10% and 90%, 20% and 90%, 30% and 90%, 40% and 90%, 40% and 80%, 45% and 75%, 1% and 14%, 2% and 14%, 3% and 14%, 4% and 14%, 5% and 14%, 5 and 13%, 5% and 12%, 5% and 11%, or 5% and 10% of the cells in a cell mixture, cell formulation, or reaction mixture.

### Enrichment of Desired Cells by Depletion of Unwanted Cells

In some embodiments, any cell in a cell mixture, cell formulation, or reaction mixture from whole blood, isolated TNCs, or isolated PBMCs can contain one or more unwanted cell populations, referred to herein as unwanted cells, that can be depleted, such that the desired cells in the cell mixture, cell formulation, or reaction mixture are enriched. In some embodiments, the unwanted cells can be depleted by negative selection prior to being contacted with a recombinant nucleic acid vector, such as a replication incompetent retroviral particle, for example as provided in methods for genetically modifying a T cell or NK cell provided herein. In other embodiments, the unwanted cells can be depleted by negative selection after the cell mixture is contacted with a recombinant nucleic acid vector, such as a replication incompetent retroviral particle, for example as provided in methods for genetically modifying a T cell or NK cell provided herein. In some embodiments, depleting the unwanted cells can be performed at the same time as any of the methods of genetic modification disclosed herein, and in illustrative embodiments genetic modification with a replication incompetent retroviral particle.

In some embodiments, the unwanted cells can include any non-T or non-NK cell. In some embodiments, the unwanted cells can include T or NK cell subsets, such as regulatory T cells or suppressor T cells. In some embodiments, the unwanted cells can include B cells. In some embodiments, the unwanted cells include monocytes. In some embodiments, the unwanted cells include granulocytes. In illustrative embodiments, the unwanted cells include cells that express the cognate antigen to a CAR that is or will be expressed on a population of the cells that will be formulated for delivery.

In further illustrative embodiments, the unwanted cells include cancer cells. Cancer cells from many types of cancer can enter the blood and could be unintentionally genetically modified at a low frequency along with the lymphocytes using the methods provided herein. In some embodiments, the cancer cell can be derived from any cancer, including, but not limited to: renal cell carcinoma, gastric cancer, sarcoma, breast cancer, lymphoma, B cell lymphoma, diffuse large B cell lymphoma (DLBCL), Hodgkin's lymphoma, non-Hodgkin's B-cell lymphoma (B-NHL), neuroblastoma, glioma, glioblastoma, medulloblastoma, colorectal cancer, ovarian cancer, prostate cancer, mesothelioma, lung cancer (e.g., small cell lung cancer), melanoma, leukemia, chronic lymphocytic leukemia (CLL), acute lymphocytic leukemia (ALL), acute myelogenous leukemia (AML), or chronic myelogenous leukemia (CML). In illustrative embodiments, the CAR-cancer cell can be derived from a B-cell lymphoma. Not to be limited by theory, a cancer cell that expresses a CAR with an ASTR that binds to an antigen expressed on its own cell surface, i.e., the CAR-expressing cancer cell is itself a target cell (CAR-cancer cell), can block CAR-T cells from binding to the antigen, also known as epitope masking, and thereby prevent the killing of the CAR-cancer cell. The CAR-cancer cell can result in recurrence of the cancer, with immunity to CAR-T, even after initial successful treatment with CAR-T (see, e.g., Ruella et al. Nat Med. 2018 October; 24(10):1499-1503). Methods and compositions provided herein for depleting unwanted cancer cells, overcome this risk posed by genetically modifying cells, such as blood cells or PBMCs, isolated from a cancer patient.

Monocytes can be depleted by incubation of the cell mixture with an immobilized monocyte-binding substrate such as a standard plastic tissue culture plate, nylon or glass wool, or sephadex resin. Not to be limited by theory, monocytes adhere preferentially to the immobilized monocyte-binding substrate versus other cells in the cell mixture, which adhere at a lower frequency or strength or do not adhere at all. In some embodiments, the incubations can be performed at 37° C. for at least 1 hour or by passing the cell mixture through a resin After the incubation, the desired non-adherent cells in suspension are collected for further processing. In illustrative embodiments of rapid ex vivo processing of lymphocytes provided herein, the whole blood, TNCs, or PBMCs are not incubated for at least 8, 7, 6, 5, 4, 3, 2, or 1 hours with an immobilized monocyte-binding substrate and the monocytes are not depleted by such an incubation.

In illustrative embodiments, methods herein include depleting unwanted cells by negative selection of cells expressing one or more surface molecules using methods known in the art for removing such cells. In illustrative embodiments, the surface molecule is a tumor-associated antigen, a tumor-specific antigen, or is otherwise expressed on cancer cells, for example, circulating tumor cells. In some embodiments, the surface molecules can include Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD19, CD20, CD22, CD23, CD24, CD27, CD30, CD33, CD34, CD37, CD38, CD40, CD44, CD44v6, CD44v7/8, CD45, CD70, CD99, CD117, CD123, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein, New York esophageal squamous cell carcinoma antigen (NYESO1), or PDL-1. In further illustrative embodiments, the surface molecule is a blood cancer antigen such as CD19, CD20, CD22, CD25, CD32, CD34, CD38, CD123, BCMA, TACI, or TIM3.

In some embodiments, unwanted cells can be depleted from a cell mixture such as whole blood, PBMCs, or TNCs, by bead or column-based separation. In these embodiments, ligand or antibody to a cell surface molecule is attached to the beads or column. In some embodiments, the antibodies attached to the beads can bind the same antigen as a CAR that is used, for example expressed by T cells and/or NK cells, in a method in which the unwanted cells are removed. In some embodiments, the antibodies attached to the beads can bind a different epitope of the same antigen as the CAR that will be expressed later in the patient. In illustrative embodiments, the antibodies attached to the beads can bind the same epitope of the same antigen as the CAR. In some embodiments, the beads can have more than one attached antibody that binds to antigens on the surface of the unwanted cells. In some embodiments, beads with different antibodies attached to them can be used in combination. In some embodiments, the beads can be magnetic beads. In some embodiments, the unwanted cells can be depleted by magnetic separation after incubation of the cell mixture with the magnetic beads with attached antibodies. In some embodiments, the beads are not magnetic.

In some embodiments, unwanted cells expressing one or more surface molecules are depleted from a cell mixture such as whole blood, PBMCs, or TNCs, by antibody coated beads and separated by size. In some embodiments the beads are polystyrene. In illustrative embodiments the beads are at least about 30 μm, about 35 μm, about 40 μm, about 50 μm, about 60 μm, about 70 μm, or about 80 μm in diameter. In some embodiments the antibody coated beads are added to the cell mixture during the time that the recombinant nucleic acid vectors, which in illustrative embodiments are replication incompetent recombinant retroviral particles, are incubated with the cell mixture. In these embodiments, a reaction mixture is formed that includes: (A) a cell mixture, such as from whole blood, enriched TNCs, or enriched PBMCs; (B) recombinant nucleic acid vectors, such as replication incompetent recombinant retroviral particles, encoding a transgene of interest, such as a CAR; and (C) antibody coated beads that bind to one or more surface molecules, or antigens, expressed on the surfaces of the unwanted cells. In some embodiments, the reaction mixture can be incubated for less than 1, 2, 3, 4, 5, 6, 7, 8, 9, 10, 15, 20, 25, 30, or 45 minutes or less than 1, 2, 3, 4, 5, 6, 7, or 8 hours. In some embodiments, after the incubation, a density-gradient centrifugation-based cell enrichment procedure can be performed to enrich total mononuclear cells depleted of the unwanted cells complexed to the antibody coated beads which will pellet. In other embodiments, the reaction mixture can be passed through a pre-filter of larger diameter mesh to deplete the unwanted cells complexed to the antibody coated beads. In some embodiments, the filter can have a pore diameter that is or is about 5 μm, 10 μm, or 15 μm smaller than the diameter of the beads. In other embodiments the beads may be magnetic beads and the pre-filter can be a magnet. Such filters can capture the unwanted cells bound to the beads and allow the desired cells to flow through downstream to the leukoreduction filter assembly which has a smaller pore diameter.

In some embodiments, unwanted cells are depleted or removed from a cell mixture that contains lymphocytes and erythrocytes, such as whole blood, by erythrocyte antibody rosetting (EA-rosetting). In EA-rosetting, antibodies that bind to antigens on the cell surfaces of unwanted cells are incubated with the cell mixture to crosslink the unwanted cells to red blood cells, which are then separated from the desired cells by density gradient centrifugation, such as provided for in RosetteSep™ kits (Stemcell Technologies). In some embodiments the antibodies that mediate EA-rosetting are added to the cell mixture during the time that the recombinant nucleic acid vectors, which in illustrative embodiments are replication incompetent recombinant retroviral particles, are incubated with the cell mixture. In illustrative embodiments, a reaction mixture is formed that includes: (A) a cell mixture of lymphocytes and erythrocytes, such as from whole blood; (B) replication incompetent recombinant retroviral particles encoding a transgene of interest, and in further illustrative embodiments a CAR; (C) a first antibody to an antigen on the surface of the unwanted cells, for example a tumor antigen such as the blood cancer antigens CD19, CD20, CD22, CD25, CD32, CD34, CD38, CD123, BCMA, TACI, or TIM3; (D) a second antibody to an antigen on the surface of an erythrocyte, such as glycophorin A; and (E) a third antibody that cross links the first and second antibodies. In further illustrative embodiments, the reaction mixture can include antibodies to more than one antigen on the surface of unwanted cells. In some embodiments, the antibodies can bind to the same antigen as does the CAR. In some embodiments, this reaction mixture is incubated for less than 1, 2, 3, 4, 5, 6, 7, 8, 9, 10, 15, 20, 25, 30, or 45 minutes or less than 1, 2, 3, 4, 5, 6, 7, or 8 hours. In illustrative embodiments, after the incubation, a density-gradient centrifugation-based PBMC enrichment procedure is performed to isolate total PBMCs minus the population depleted or removed by EA-rosetting which will pellet with the erythrocytes.

As discussed above, genetic modification of cancer cells with a recombinant nucleic acid vector encoding an engineered T cell receptor or a CAR can be minimized during cell processing by the enrichment of T and/or NK cells by including a step of positive selection or depletion of the cancer cells by negative selection from the cell mixture in methods provided herein, prior to formulation and/or delivery to a subject. Several additional methods to reduce the potential effects of cancer cells genetically modified with an engineered T cell receptor construct or a CAR construct are disclosed herein. For example, T cell-specific promoters (disclosed elsewhere herein) can be used to express the CAR and can help prevent non-T cells that contain an exogenous nucleic acid(s) encoding a CAR from actually expressing the CAR. Thus, the antigen will not be masked by a CAR expressed in cis, and CAR-T cells can bind to and kill the target cell containing an exogenous nucleic acid(s) encoding the CAR. Furthermore, using a T cell-specific promoter for expressing an engineered T cell receptor or a CAR, helps to reduce, minimize, or in illustrative embodiments substantially eliminate, or even eliminate expression of the engineered T cell receptor or CAR in a encapsulated nucleic acid vector such as a RIR retroviral particle or a virus-like particle because of reduced, low, negligible, substantially no, or no expression of the engineered T cell receptor or CAR in a cell line used to make the encapsulated nucleic acid vector. In illustrative embodiments, such expression is reduced, substantially eliminated, or eliminated on the surface of the encapsulated nucleic acid vector (e.g. RIR particle or virus-like particle).

Another method to reduce the potential effects of CAR-cancer cells is to use two or more separate CARs, and in illustrative embodiments, two CARs expressed in two populations of cells, to kill target cells that could mask one of the epitopes. A population of cells, such as blood cells or PBMCs, are genetically modified separately so each population expresses either a first CAR or a second CAR. In illustrative embodiments, a target cell expressing the first or second CAR does not mask the epitope that the second and first CAR, respectively, bind to. Therefore, a target cell expressing the first or second CAR can be killed by an effector T or NK cell expressing the second or first CAR, respectively. In some embodiments, the first and second CARs can bind to different epitopes of the same antigen expressed on a target cell. In other embodiments, the first and second CARs can bind to different antigens expressed on the same target cell, including any of the antigens disclosed elsewhere herein. In some embodiments, the first and second CARs can bind to different epitopes of, or different antigens selected from CD19, CD20, CD22, CD25, CD32, CD34, CD38, CD123, BCMA, TACI or TIM3. In further illustrative embodiments, the first CAR can bind to CD19 and the second CAR can bind to CD22, both of which are expressed on B cells. In other embodiments, the CAR can be an extracellular ligand of a cancer antigen. In illustrative embodiments, the modified cell populations are formulated separately. In some embodiments, the separate cell formulations are introduced or reintroduced back into the subject at different sites in the body. In some embodiments, separate cell formulations are separately introduced or reintroduced back into the subject at the same site. In other embodiments, the modified cell populations are combined into one formulation that is optionally introduced or reintroduced back into the subject together at the same site. In illustrative embodiments wherein the cell populations are combined, the cell populations are not combined until after a washing step in which the cells are washed away from the recombinant nucleic acid vectors. By this method of using two or more distinct CARs, a CAR-cancer cell expressing a first or second CAR that binds and masks its cognate epitope in cis, will be killed by a CAR-T cell expressing the second or first CAR, respectively.

In some embodiments, the unwanted cells can be depleted such that the unwanted cells comprise at most 1%, 2%, 3%, 4%, 5%, 6%, 7%, 8%, 9%, 10%, 15%, 20%, 30%, 40%, 50%, 60%, 70%, 80%, 90%, 95%, 96%, 97%, 98%, or 99% of the cells in a cell mixture, cell formulation, or reaction mixture. In some embodiments, the unwanted cells can be depleted such that the unwanted cells comprise between 1%, 2%, 3%, 4%, 5%, 6%, 7%, 8%, 9%, 10%, 20%, 30%, or 40% of the cells in a cell mixture, cell formulation, or reaction mixture on the low end of the range and 10%, 20%, 30%, 40%, 50%, 60%, 70%, 80%, 90%,95%, 96%, 97%, 98%, or 99% of the cells in a cell mixture, cell formulation, or reaction mixture on the high end of the range. In some embodiments, the unwanted cells can be depleted such that the unwanted cells comprise between 10% and 90%, 20% and 90%, 30% and 90%, 40% and 90%, 40% and 80%, 45% and 75%, 1% and 14%, 2% and 14%, 3% and 14%, 4% and 14%, 5% and 14%, 5 and 13%, 5% and 12%, 5% and 11%, or 5% and 10% of the cells in a cell mixture, cell formulation, or reaction mixture.

### Engineered Signaling Polypeptide(s)

In some embodiments, the replication incompetent recombinant retroviral particles used to contact T cells and/or NK cells have a polynucleotide or nucleic acid having one or more transcriptional units that encode one or more engineered signaling polypeptides. In some embodiments, an engineered signaling polypeptide includes any combination of an extracellular domain (e.g., an antigen-specific targeting region or ASTR), a stalk and a transmembrane domain, combined with one or more intracellular activating domains, optionally one or more modulatory domains (such as a co-stimulatory domain), and optionally one or more T cell survival motifs. In illustrative embodiments, at least one, two, or all of the engineered signaling polypeptides is a chimeric antigen receptor (CAR) or a lymphoproliferative element (LE) such as a chimeric lymphoproliferative element (CLE). In some embodiments, at least one, two, or all of the engineered signaling polypeptides is an engineered T cell receptor (TCR). In some embodiments, when two signaling polypeptides are utilized, one encodes a lymphoproliferative element and the other encodes a chimeric antigen receptor (CAR) that includes an antigen-specific targeting region (ASTR), a transmembrane domain, and an intracellular activating domain. For any domain of an engineered signaling polypeptide disclosed herein, exemplary sequences can be found in WO2019/055946, incorporated herein in its entirety by reference. A skilled artisan will recognize that such engineered polypeptides can also be referred to as recombinant polypeptides. The engineered signaling polypeptides, such as CARs, engineered TCRs, LEs, and CLEs provided herein, are typically transgenes with respect to lymphocytes, especially T cells and NK cells, and most especially T cells and/or NK cells that are engineered using methods and compositions provided herein, to express such signaling polypeptides.

### Extracellular Domain

In some embodiments, an engineered signaling polypeptide includes an extracellular domain that is a member of a specific binding pair. For example, in some embodiments, the extracellular domain can be the extracellular domain of a cytokine receptor, or a mutant thereof, or a hormone receptor, or a mutant thereof. Such mutant extracellular domains in some embodiments have been reported to be constitutively active when expressed at least in some cell types. In illustrative embodiments, such extracellular and transmembrane domains do not include a ligand binding region. It is believed that such domains do not bind a ligand when present in an engineered signaling polypeptide and expressed in B cells, T cells, and/or NK cells. Mutations in such receptor mutants can occur in the extracellular juxtamembrane region. Not to be limited by theory, a mutation in at least some extracellular domains (and some extracellular-transmembrane domains) of engineered signaling polypeptides provided herein, are responsible for signaling of the engineered signaling polypeptide in the absence of ligand, by bringing activating chains together that are not normally together. Further embodiments regarding extracellular domains that comprise mutations in extracellular domains can be found, for example, in the Lymphoproliferative Element section herein.

In certain illustrative embodiments, the extracellular domain comprises a dimerizing motif. In an illustrative embodiment the dimerizing motif comprises a leucine zipper. In some embodiments, the leucine zipper is from a jun polypeptide, for example c-jun. Further embodiments regarding extracellular domains that comprise a dimerizing motif can be found, for example, in the Lymphoproliferative Element section herein.

In certain embodiments, the extracellular domain is an antigen-specific targeting region (ASTR), sometimes called an antigen binding domain herein. Specific binding pairs include, but are not limited to, antigen-antibody binding pairs; ligand-receptor binding pairs; and the like. Thus, a member of a specific binding pair suitable for use in an engineered signaling polypeptide of the present disclosure includes an ASTR that is an antibody, an antigen, a ligand, a receptor binding domain of a ligand, a receptor, a ligand binding domain of a receptor, and an alternative non-antibody scaffold, also referred to herein as an antibody mimetic. In any of the aspects or embodiments provided herein that include an ASTR, the ASTR can be a suitable antibody mimetic. In some embodiments, the antibody mimetic can be an affibody, an afflilin, an affimer, an affitin, an alphabody, an alphamab, an anticalin, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain In any of the aspects or embodiments provided herein that include an ASTR that is an antibody, for example, an scFv, a suitable antibody mimetic can be used instead of the antibody.

An ASTR suitable for use in an engineered signaling polypeptide of the present disclosure can be any antigen-binding polypeptide. In certain embodiments, the ASTR is an antibody such as a full-length antibody, a single-chain antibody, a Fab fragment, a Fab′ fragment, a (Fab′)2 fragment, a Fv fragment, and a divalent single-chain antibody or a diabody.

In some embodiments, the ASTR is a single chain Fv (scFv). In some embodiments, the heavy chain is positioned N-terminal of the light chain in the engineered signaling polypeptide. In other embodiments, the light chain is positioned N-terminal of the heavy chain in the engineered signaling polypeptide. In any of the disclosed embodiments, the heavy and light chains can be separated by a linker as discussed in more detail herein. In any of the disclosed embodiments, the heavy or light chain can be at the N-terminus of the engineered signaling polypeptide and is typically C-terminal of another domain, such as a signal sequence or peptide.

Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use with the engineered signaling polypeptides and methods using the engineered signaling polypeptides of the present disclosure. In some instances, T cell receptor (TCR) based recognition domains.

Naturally-occurring T cell receptors include an α-subunit and a β-subunit, separately produced by unique recombination events in a T cell's genome. Libraries of TCRs may be screened for their selectivity to a target antigen, for example, any of the antigens disclosed herein. Screens of natural and/or engineered TCRs can identify TCRs with high avidities and/or reactivities towards a target antigen. Such TCRs can be selected, cloned, and a polynucleotide encoding such a TCR can be included in a replication incompetent recombinant retroviral particle to genetically modify a lymphocyte, or in illustrative embodiments, T cell or NK cell, such that the lymphocyte expresses the engineered TCR. In some embodiments, the TCR can be a single chain TCR (scTv, single chain two-domain TCR containing VαVβ).

Certain embodiments for any aspect or embodiment herein that includes a CAR, include CARs having extracellular domains engineered to co-opt the endogenous TCR signaling complex and CD3Z signaling pathway. In one embodiment, a chimeric antigen receptor ASTR is fused to one of the endogenous TCR complex chains (e.g., TCR alpha, CD3E etc.) to promote incorporation into the TCR complex and signaling through the endogenous CD3Z chains. In other embodiments, a CAR contains a first scFv or protein that binds to the TCR complex and a second scFv or protein that binds to the target antigen (e.g., tumor antigen). In another embodiment, the TCR can be a single chain TCR (scTv, single chain two-domain TCR containing VαVβ). Finally, scFv's may also be generated to recognize the specific MHC/peptide complex, thereby acting as a surrogate TCR. Such peptide/MHC scFv-binders may be used in many similar configurations as CARs.

In some embodiments, the ASTR can be multispecific, e.g., bispecific antibodies. Multispecific antibodies have binding specificities for at least two different sites. In certain embodiments, one of the binding specificities is for one target antigen and the other is for another target antigen. In certain embodiments, bispecific antibodies may bind to two different epitopes of a target antigen. Bispecific antibodies may also be used to localize cytotoxic agents to cells which express a target antigen. Bispecific antibodies can be prepared as full-length antibodies or antibody fragments.

An ASTR suitable for use in an engineered signaling polypeptide of the present disclosure, or an engineered TCR, can have a variety of antigen-binding specificities. In some cases, the antigen-binding domain is specific for an epitope present in an antigen that is expressed by (synthesized by) a target cell. In one example, the target cell is a cancer cell associated antigen. The cancer cell associated antigen can be an antigen associated with, e.g., a breast cancer cell, a B cell lymphoma cell, as a diffuse large B cell lymphoma (DLBCL) cell, a Hodgkin lymphoma cell, an ovarian cancer cell, a prostate cancer cell, a mesothelioma, a lung cancer cell (e.g., a small cell lung cancer cell), a lymphoma cell, a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a leukemia cell, a chronic myelogenous leukemia (CML) cell, a chronic lymphocytic leukemia (CLL) cell, an acute myelogenous leukemia (AML) cell, an acute lymphocytic leukemia (ALL) cell, a neuroblastoma cell, a glioma, a glioblastoma, a medulloblastoma, a colorectal cancer cell, etc. A cancer cell associated antigen may also be expressed by a non-cancerous cell. In some embodiments, the cancer cell is a PDL-1 positive cancer cell. In illustrative embodiments, the cancer cell is a PDL-1 positive DLBCL cell. In some embodiments, the cancer cell is a PDL-1 negative cell. In illustrative embodiments, the cancer cell is a PDL-1 negative DLBCL cell.

Non-limiting examples of antigens to which an ASTR of an engineered signaling polypeptide can bind, or an engineered T cell receptor can bind, include, e.g., In any of the aspects or embodiments herein that include an ASTR, the antigen can be a tumor-associated antigen or a tumor-specific antigen. In some embodiments, the tumor-associated antigen or tumor-specific antigen is Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD19, CD20, CD22, CD23, CD24, CD27, CD30, CD33, CD34, CD37, CD38, CD40, CD44, CD44v6, CD44v7/8, CD45, CD70, CD99, CD117, CD123, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), PDL-1 and the like.

In some embodiments, a member of a specific binding pair suitable for use in an engineered signaling polypeptide is an ASTR that is a ligand for a receptor. Ligands include, but are not limited to, hormones (e.g., erythropoietin, growth hormone, leptin, etc.); cytokines (e.g., interferons, interleukins, certain hormones, etc.); growth factors (e.g., heregulin; vascular endothelial growth factor (VEGF); and the like); an integrin-binding peptide (e.g., a peptide comprising the sequence Arg-Gly-Asp (SEQ ID NO:1)); and the like.

Where the member of a specific binding pair in an engineered signaling polypeptide is a ligand, the engineered signaling polypeptide can be activated in the presence of a second member of the specific binding pair, where the second member of the specific binding pair is a receptor for the ligand. For example, where the ligand is VEGF, the second member of the specific binding pair can be a VEGF receptor, including a soluble VEGF receptor.

As noted above, in some cases, the member of a specific binding pair that is included in an engineered signaling polypeptide is an ASTR that is a receptor, e.g., a receptor for a ligand, a co-receptor, etc. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); CD27; a natural cytotoxicity receptor (NCR) (e.g., NKP30 (NCR3/CD337) polypeptide (receptor for HLA-B-associated transcript 3 (BAT3) and B7-H6); etc.); etc.

In certain embodiments of any of the aspects provided herein that include an ASTR, the ASTR can be directed to an intermediate protein that links the ASTR with a target molecule expressed on a target cell. The intermediate protein may be endogenously expressed or introduced exogenously and may be natural, engineered, or chemically modified. In certain embodiments the ASTR can be an anti-tag ASTR such that at least one tagged intermediate, typically an antibody-tag conjugate, is included between a tag recognized by the ASTR and a target molecule, typically a protein target, expressed on a target cell. Accordingly, in such embodiments, the ASTR binds a tag and the tag is conjugated to an antibody directed against an antigen on a target cell, such as a cancer cell. Non-limiting examples of tags include fluorescein isothiocyanate (FITC), streptavidin, biotin, histidine, dinitrophenol, peridinin chlorophyll protein complex, green fluorescent protein, phycoerythrin (PE), horse radish peroxidase, palmitoylation, nitrosylation, alkaline phosphatase, glucose oxidase, and maltose binding protein. As such, the ASTR comprises a molecule that binds the tag.

### Stalk

In some embodiments, the engineered signaling polypeptide includes a stalk which is located in the portion of the engineered signaling polypeptide lying outside the cell and interposed between the ASTR and the transmembrane domain In some embodiments, the stalk has at least 85, 90, 95, 96, 97, 98, 99, or 100% identity to a wild-type CD8 stalk region (TTTPAPRPPTPAPTIASQPLSLRPEACRPAAGG AVHTRGLDFA (SEQ ID NO:2), has at least 85, 90, 95, 96, 97, 98, 99, or 100% identity to a wild-type CD28 stalk region (FCKIEVMYPPPYLDNEKSNGTIIHVKGKHLCPSPLFPGPSKP (SEQ ID NO:3)), or has at least 85, 90, 95, 96, 97, 98, 99, or 100% identity to a wild-type immunoglobulin heavy chain stalk region. In an engineered signaling polypeptide, the stalk employed allows the antigen-specific targeting region, and typically the entire engineered signaling polypeptide, to retain increased binding to a target antigen.

The stalk region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

In some embodiments, the stalk of an engineered signaling polypeptide includes at least one cysteine. For example, In some embodiments, the stalk can include the sequence Cys-Pro-Pro-Cys (SEQ ID NO:4). If present, a cysteine in the stalk of a first engineered signaling polypeptide can be available to form a disulfide bond with a stalk in a second engineered signaling polypeptide.

Stalks can include immunoglobulin hinge region amino acid sequences that are known in the art; see, e.g., Tan et al. (1990) Proc. Natl. Acad. Sci. USA 87:162; and Huck et al. (1986) Nucl. Acids Res. 14:1779. As non-limiting examples, an immunoglobulin hinge region can include a domain with at least 50, 60, 70, 75, 80, 85, 90, 95, 96, 97, 98, 99 or 100% sequence identity to a stretch of at least 10, 15, 20, or all of the amino acids of any of the following amino acid sequences: DKTHT (SEQ ID NO:5); CPPC (SEQ ID NO:4); CPEPKSCDTPPPCPR (SEQ ID NO:6) (see, e.g., Glaser et al. (2005) J. Biol. Chem. 280:41494); ELKTPLGDTTHT (SEQ ID NO:7); KSCDKTHTCP (SEQ ID NO:8); KCCVDCP (SEQ ID NO:9); KYGPPCP (SEQ ID NO:10); EPKSCDKTHTCPPCP (SEQ ID NO:11) (human IgG1 hinge); ERKCCVECPPCP (SEQ ID NO:12) (human IgG2 hinge); ELKTPLGDTTHTCPRCP (SEQ ID NO:13) (human IgG3 hinge); SPNMVPHAHHAQ (SEQ ID NO:14) (human IgG4 hinge); and the like. The stalk can include a hinge region with an amino acid sequence of a human IgG1, IgG2, IgG3, or IgG4, hinge region. The stalk can include one or more amino acid substitutions and/or insertions and/or deletions compared to a wild-type (naturally-occurring) hinge region. For example, His229 of human IgG 1 hinge can be substituted with Tyr, so that the stalk includes the sequence EPKSCDKTYTCPPCP (SEQ ID NO:15), (see, e.g., Yan et al. (2012) J. Biol. Chem. 287:5891). The stalk can include an amino acid sequence derived from human CD8; e.g., the stalk can include the amino acid sequence: TTTPAPRPPTPAPTIASQPLSLRPEACRPAAGGAVHTRGLDFACD (SEQ ID NO:16), or a variant thereof.

### Transmembrane Domain

An engineered signaling polypeptide of the present disclosure can include transmembrane domains for insertion into a eukaryotic cell membrane. The transmembrane domain can be interposed between the ASTR and the co-stimulatory domain The transmembrane domain can be interposed between the stalk and the co-stimulatory domain, such that the chimeric antigen receptor includes, in order from the amino terminus (N-terminus) to the carboxyl terminus (C-terminus): an ASTR; a stalk; a transmembrane domain; and an activating domain

Any transmembrane (TM) domain that provides for insertion of a polypeptide into the cell membrane of a eukaryotic (e g , mammalian) cell is suitable for use in aspects and embodiments disclosed herein. In some embodiments, the TM domain for any aspect provided herein that includes a CAR can include a transmembrane domain from BAFFR, C3Z, CEACAM1, CD2, CD3A, CD3B, CD3D, CD3E, CD3G, CD3Z, CD4, CDS, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof.

Non-limiting examples of TM domains suitable for any of the aspects or embodiments provided herein, include a domain with at least 50, 60, 70, 75, 80, 85, 90, 95, 96, 97, 98, 99 or 100% sequence identity to a stretch of at least 10, 15, 20, or all of the amino acids of any of the following TM domains or combined stalk and TM domains: a) CD8 alpha TM (SEQ ID NO:17); b) CD8 beta TM (SEQ ID NO:18); c) CD4 stalk (SEQ ID NO:19); d) CD3Z TM (SEQ ID NO:20); e) CD28 TM (SEQ ID NO:21); f) CD134 (OX40) TM: (SEQ ID NO:22); g) CD7 TM (SEQ ID NO:23); h) CD8 stalk and TM (SEQ ID NO:24); and i) CD28 stalk and TM (SEQ ID NO:25).

As non-limiting examples, a transmembrane domain of an aspect of the invention can have at least 80%, 90%, or 95% or can have 100% sequence identity to the SEQ ID NO:17 transmembrane domain, or can have 100% sequence identity to any of the transmembrane domains from the following genes respectively: the CD8 beta transmembrane domain, the CD4 transmembrane domain, the CD3 zeta transmembrane domain, the CD28 transmembrane domain, the CD134 transmembrane domain, or the CD7 transmembrane domain

### Intracellular Activating Domain

Intracellular activating domains suitable for use in an engineered signaling polypeptide of the present disclosure when activated, typically induce the production of one or more cytokines; increase cell death; and/or increase proliferation of CD8⁺ T cells, CD4⁺ T cells, NKT cells, γδ T cells, and/or neutrophils. Activating domains can also be referred to as activation domains herein. Activating domains can be used in CARs or in lymphoproliferative elements provided herein.

In some embodiments, the intracellular activating domain includes at least one (e.g., one, two, three, four, five, six, etc.) ITAM motifs as described below. In some embodiments, an intracellular activating domain of an aspect of the invention can have at least 80%, 90%, or 95% or can have 100% sequence identity to the CD3Z, CD3D, CD3E, CD3G, CD79A, CD79B, DAP12, FCER1G, FCGR2A, FCGR2C, DAP10/CD28, ZAP70, NKp30 (B7-H6), NKG2D, NKp44, NKp46, FcR gamma (FCER1G), FcR beta (FCER1B), FcgammaRI, FcgammaRIIA, FcgammaRIIC, FcgammaRIIIA, and FcRL5 domains as described below.

Intracellular activating domains suitable for use in an engineered signaling polypeptide of the present disclosure include immunoreceptor tyrosine-based activation motif (ITAM)-containing intracellular signaling polypeptides. An ITAM motif is YX₁X₂L/I, where X₁ and X₂ are independently any amino acid. In some embodiments, the intracellular activating domain of an engineered signaling polypeptide includes 1, 2, 3, 4, or 5 ITAM motifs. In some embodiments, an ITAM motif is repeated twice in an intracellular activating domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid. In some embodiments, the intracellular activating domain of an engineered signaling polypeptide includes 3 ITAM motifs.

A suitable intracellular activating domain can be an ITAM motif-containing portion that is derived from a polypeptide that contains an ITAM motif. For example, a suitable intracellular activating domain can be an ITAM motif-containing domain from any ITAM motif-containing protein. Thus, a suitable intracellular activating domain need not contain the entire sequence of the entire protein from which it is derived. Examples of suitable ITAM motif-containing polypeptides include, but are not limited to: CD3Z (CD3 zeta); CD3D (CD3 delta); CD3E (CD3 epsilon); CD3G (CD3 gamma); CD79A (antigen receptor complex-associated protein alpha chain); CD79B (antigen receptor complex-associated protein beta chain) DAP12; and FCER1G (Fc epsilon receptor I gamma chain).

In some embodiments, an intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all the amino acids in the following ITAM motif-containing polypeptides or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of any of the following ITAM motif-containing polypeptides: CD3 zeta chain (also known as CD3Z, T cell receptor T3 zeta chain, CD247, CD3-ZETA, CD3H, CD3Q, T3Z, TCRZ, etc.) with exemplary sequences MKWKALFTAAILQAQLPITEAQSFGLLDPKLCYLLDGILFIYGVILTALFLRVKFSRSADAPAYQQ GQNQL[YNELNLGRREEYDVL]DKRRGRDPEMGGKPRRKNPQEGL[YNELQKDKMAEAYSEI]G MKGERRRGKGHDGL[YQGLSTATKDTYDAL]HMQALPPR (SEQ ID NO:26), MKWKALFTAAILQAQLPITEAQSFGLLDPKLCYLLDGILFIYGVILTALFLRVKFSRSADAPAYQQ GQNQL[YNELNLGRREEYDVL]DKRRGRDPEMGGKPQRRKNPQEGL[YNELQKDKMAEAYSEI] GMKGERRRGKGHDGL[YQGLSTATKDTYDAL]HMQALPPR (SEQ ID NO:27), RVKFSRSADAPAYQQGQNQL[YNELNLGRREEYDVL]DKRRGRDPEMGGKPRRKNPQEGL[YNE LQKDKMAEAYSEI]GMKGERRRGKGHDGL[YQGLSTATKDTYDAL]HMQALPPR (SEQ ID NO:28), RVKFSRSADAPAYQQGQNQL[YNELNLGRREEYDVL]DKRRGRDPEMGGKPQRRKNPQEGL[YN ELQKDKMAEAYSEI]GMKGERRRGKGHDGL[YQGLSTATKDTYDAL]HMQALPPR (SEQ ID NO:29), NQL[YNELNLGRREEYDVL]DKR SEQ ID NO:30); EGL[YNELQKDKMAEAYSEI]GMK (SEQ ID NO:31), and DGL[YQGLSTATKDTYDAL]HMQ (SEQ ID NO:32); T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.) with exemplary sequences: MEHSTFLSGLVLATLLSQVSPFKIPIEELEDRVFVNCNTSITWVEGTVGTLLSDITRLDLGKRILDP RGIYRCNGTDIYKDKESTVQVHYRMCQSCVELDPATVAGIIVTDVIATLLLALGVFCFAGHETGR LSGAADTQALLRNDQV[YQPLRDRDDAQYSHL]GGNWARNK (SEQ ID NO:33), MEHSTFLSGLVLATLLSQVSPFKIPIEELEDRVFVNCNTSITWVEGTVGTLLSDITRLDLGKRILDP RGIYRCNGTDIYKDKESTVQVHYRTADTQALLRNDQV[YQPLRDRDDAQYSHL]GGNWARNK (SEQ ID NO:34), and DQV[YQPLRDRDDAQYSHL]GGN (SEQ ID NO:35); T cell surface glycoprotein CD3 epsilon chain (also known as CD3e, T cell surface antigen T3/Leu-4 epsilon chain, T cell surface glycoprotein CD3 epsilon chain, AI504783, CD3, CD3epsilon, T3e, etc.) with exemplary sequences: MQSGTHWRVLGLCLLSVGVWGQDGNEEMGGITQTPYKVSISGTTVILTCPQYPGSEILWQHNDK NIGGDEDDKNIGSDEDHLSLKEFSELEQSGYYVCYPRGSKPEDANFYLYLRARVCENCMEMDMS VATIVIVDICITGGLLLLVYYWSKNRKAKAKPVTRGAGAGGRQRGQNKERPPPVPNPD[YEPIRKGQRDLYSGL]NQRRI (SEQ ID NO:36) and NPD[YEPIRKGQRDLYSGL]NQR (SEQ ID NO:37); T cell surface glycoprotein CD3 gamma chain (also known as CD3G, T cell receptor T3 gamma chain, CD3-GAMMA, T3G, gamma polypeptide (TiT3 complex), etc.) with exemplary sequences: MEQGKGLAVLILAIILLQGTLAQSIKGNHLVKVYDYQEDGSVLLTCDAEAKNITWFKDGKMIGF LTEDKKKWNLGSNAKDPRGMYQCKGSQNKSKPLQVYYRMCQNCIELNAATISGFLFAEIVSIFV LAVGVYFIAGQDGVRQSRASDKQTLLPNDQL[YQPLKDREDDQYSHL]QGNQLRRN (SEQ ID NO:38) and DQL[YQPLKDREDDQYSHL]QGN (SEQ ID NO:39); CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.) with exemplary sequences: MPGGPGVLQALPATIFLLFLLSAVYLGPGCQALWMHKVPASLMVSLGEDAHFQCPHNSSNNAN VTWWRVLHGNYTWPPEFLGPGEDPNGTLIIQNVNKSHGGIYVCRVQEGNESYQQSCGTYLRVR QPPPRPFLDMGEGTKNRIITAEGIILLFCAVVPGTLLLFRKRWQNEKLGLDAGDEYEDENL[YEGL NLDDCSMYEDI]SRGLQGTYQDVGSLNIGDVQLEKP (SEQ ID NO:40), MPGGPGVLQALPATIFLLFLLSAVYLGPGCQALWMHKVPASLMVSLGEDAHFQCPHNSSNNAN VTWWRVLHGNYTWPPEFLGPGEDPNEPPPRPFLDMGEGTKNRIITAEGIILLFCAVVPGTLLLFRK RWQNEKLGLDAGDEYEDENL[YEGLNLDDCSMYEDI]SRGLQGTYQDVGSLNIGDVQLEKP (SEQ ID NO:41), and ENL[YEGLNLDDCSMYEDI]SRG (SEQ ID NO:42); CD79B with exemplary sequence: LDKDDSKAGMEEDHT[YEGLDIDQTATYEDI]VTLRTGEVKWSVGEHPGQE (SEQ ID NO:211); DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.) with exemplary sequences: MGGLEPCSRLLLLPLLLAVSGLRPVQAQAQSDCSCSTVSPGVLAGIVMGDLVLTVLIALAVYFLG RLVPRGRGAAEAATRKQRITETESP[YQELQGQRSDVYSDL]NTQRPYYK (SEQ ID NO:43), MGGLEPCSRLLLLPLLLAVSGLRPVQAQAQSDCSCSTVSPGVLAGIVMGDLVLTVLIALAVYFLG RLVPRGRGAAEATRKQRITETESP[YQELQGQRSDVYSDL]NTQ (SEQ ID NO:44), MGGLEPCSRLLLLPLLLAVSDCSCSTVSPGVLAGIVMGDLVLTVLIALAVYFLGRLVPRGRGAAE AATRKQRITETESP[YQELQGQRSDVYSDL]NTQRPYYK (SEQ ID NO:45), MGGLEPCSRLLLLPLLLAVSDCSCSTVSPGVLAGIVMGDLVLTVLIALAVYFLGRLVPRGRGAAE ATRKQRITETESP[YQELQGQRSDVYSDL]NTQRPYYK (SEQ ID NO:46), and ESP[YQELQGQRSDVYSDL]NTQ (SEQ ID NO:47); and FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.) with exemplary sequences: MIPAVVLLLLLLVEQAAALGEPQLCYILDAILFLYGIVLTLLYCRLKIQVRKAAITSYEKSDGV[YT GLSTRNQETYETL]KHEKPPQ (SEQ ID NO:48) and DGV[YTGLSTRNQETYETL]KHE (SEQ ID NO:49), where the ITAM motifs are set out in brackets.

Intracellular activating domains suitable for use in an engineered signaling polypeptide of the present disclosure include a DAP10/CD28 type signaling chain. An example of a DAP10 signaling chain is the amino acid SEQ ID NO:50. In some embodiments, a suitable intracellular activating domain includes a domain with at least 50%, 60%, 70%, 75%, 80%, 85%, 90%, 95%, 96%, 97%, 98%, 99% or 100% sequence identity to a stretch of at least 10, 15, 20, or all amino acids in SEQ ID NO:50.

An example of a CD28 signaling chain is the amino acid sequence is SEQ ID NO:51. In some embodiments, a suitable intracellular domain includes a domain with at least 50%, 60%, 70%, 75%, 80%, 85%, 90%, 95%, 96%, 97%, 98%, 99% or 100% sequence identity to a stretch of at least 10, 15, 20, or all amino acids of SEQ ID NO:51.

Intracellular activating domains suitable for use in an engineered signaling polypeptide of the present disclosure include a ZAP70 polypeptide, For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 300 amino acids to about 400 amino acids, from about 400 amino acids to about 500 amino acids, or from about 500 amino acids to 619 amino acids, of SEQ ID NO:52.

### Modulatory Domains

Modulatory domains can change the effect of the intracellular activating domain in the engineered signaling polypeptide, including enhancing or dampening the downstream effects of the activating domain or changing the nature of the response. Modulatory domains suitable for use in an engineered signaling polypeptide of the present disclosure include co-stimulatory domains. A modulatory domain suitable for inclusion in the engineered signaling polypeptide can have a length of from about 30 amino acids to about 70 amino acids (aa), e.g., a modulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, modulatory domain can have a length of from about 70 aa to about 100 aa, from about 100 aa to about 200 aa, or greater than 200 aa.

Co-stimulatory domains typically enhance and/or change the nature of the response to an activation domain Co-stimulatory domains suitable for use in an engineered signaling polypeptide of the present disclosure are generally polypeptides derived from receptors. In some embodiments, co-stimulatory domains homodimerize. A subject co-stimulatory domain can be an intracellular portion of a transmembrane protein (i.e., the co-stimulatory domain can be derived from a transmembrane protein). In some embodiments, any of the CAR provided herein can include a costimulatory domain In some embodiments, the co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids or an intracellular domain of 4-1BB (CD137), B7-H3, B7-HCDR3, BAFFR, BTLA, C100 (SEMA4D), CD2, CD4, CD7, CD8A, CD8B, CD11A, CD11B, CD11C, CD11D, CD18, CD19, CD27, CD28, CD28 deleted for Lck binding (ICΔ), CD29, CD30, CD40, CD49A, CD49D, CD49F, CD69, CD84, CD96 (Tactile), CD103, CD160 (BY55), CD162 (SELPLG), CD226 (DNAM1), CD229 (Ly9), a ligand that specifically binds with CD83, CDS, CEACAM1, CRLF2, CRTAM, CSF2RA, CSF2RB, CSF3R, EPOR, Fc receptor gamma chain, Fc receptor ε chain, FCGRA2, GADS, GHR, GITR, HVEM, IA4, ICAM-1, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA4, ITGA6, ITGAD, ITGAE, ITGAL, ITGAM, ITGAX, ITGB1, ITGB2, ITGB7, LAT, LEPR, LFA-1 (CD11a/CD18), LIGHT, LIFR, LMP1, LTBR, MPL, MYD88, NKG2C, NKP80 (KLRF1), OSMR, OX40, PD-1, PRLR, PSGL1, PAG/Cbp, SLAM (SLAMF1, CD150, IPO-3), SLAMF4 (C244, 2B4), SLAMF6 (NTB-A, Ly108), SLAMF7, SLAMF8 (BLAME), SLP-76, TILR2, TILR4, TILR7, TILR9, TNFR2, TNFRSF4, TNFRSF8, TNFRSF9, TNFRSF14, TNFRSF18, TRANCE/RANKL, VLA1, or VLA-6,or functional mutants and/or fragments thereof.

A co-stimulatory domain suitable for inclusion in an engineered signaling polypeptide can have a length of from about 30 amino acids to about 70 amino acids (aa), e.g., a co-stimulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, the co-stimulatory domain can have a length of from about 70 aa to about 100 aa, from about 100 aa to about 200 aa, or greater than 200 aa.

In some embodiments, a co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all the amino acids or from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, or from about 160 aa to about 185 aa (depending on how long the intracellular portion of the protein is) of an intracellular portion of: CD137 (also known as TNFRSF9; CD137; 4-1BB; CDw137; ILA; etc.) for example SEQ ID NO:53, CD28 (also known as Tp44) for example SEQ ID NO:54, CD28 deleted for Lck binding (ICΔ) for example SEQ ID NO:55, ICOS (also known as AILIM, CD278, and CVID1) for example SEQ ID NO:56, OX40 (also known as TNFRSF4, RP5-902P8.3, ACT35, CD134, OX-40, TXGP1L) for example SEQ ID NO:57, CD27 (also known as S 152, T 14, TNFRSF7, and Tp55) for example SEQ ID NO:58, BTLA (also known as BTLA1 and CD272) for example SEQ ID NO:59, CD30 (also known as TNFRSF8, D1S166E, and Ki-1), for example SEQ ID NO:60, GITR (also known as TNFRSF18, RP5-902P8.2, AITR, CD357, and GITR-D), for example SEQ ID NO:61, or HVEM (also known as TNFRSF14, RP3-395M20.6, ATAR, CD270, HVEA, HVEM, LIGHTR, and TR2), for example SEQ ID NO:62. OX40 contains a p85 PI3K binding motif at residues 34-57 and a TRAF binding motif at residues 76-102, each of SEQ ID NO: 296 (of Table 1). In some embodiments, the costimulatory domain can include the p85 PI3K binding motif of OX40. In some embodiments, the costimulatory domain can include the TRAF binding motif of OX40. Lysines corresponding to amino acids 17 and 41 of SEQ ID NO: 296 are potentially negative regulatory sites that function as parts of ubiquitin targeting motifs. In some embodiments, one or both of these Lysines in the costimulatory domain of OX40 are mutated Arginines or another amino acid.

### Linker

In some embodiments, the engineered signaling polypeptide includes a linker between any two adjacent domains. For example, a linker can be between the transmembrane domain and the first co-stimulatory domain As another example, the ASTR can be an antibody and a linker can be between the heavy chain and the light chain. As another example, a linker can be between the ASTR and the transmembrane domain and a co-stimulatory domain As another example, a linker can be between the co-stimulatory domain and the intracellular activating domain of the second polypeptide. As another example, the linker can be between the ASTR and the intracellular signaling domain.

The linker peptide may have any of a variety of amino acid sequences. Proteins can be joined by a spacer peptide, generally of a flexible nature, although other chemical linkages are not excluded. A linker can be a peptide of between about 1 and about 100 amino acids in length, or between about 1 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, (GSGGS)ₙ, (GGS)ₙ, (GGGS)ₙ, and (GGGGS)ₙ where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGGGSGGGGSGGGGS (SEQ ID NO:63), GGGGSGGGGSGGGGSGGGGSGGGGSGGGGS (SEQ ID NO:64), GGGGSGGGSGGGGS (SEQ ID NO:65), GGSG (SEQ ID NO:66), GGSGG (SEQ ID NO:67), GSGSG (SEQ ID NO:68), GSGGG (SEQ ID NO:69), GGGSG (SEQ ID NO:70), GSSSG (SEQ ID NO:71), GGGGSGGGGSGGGGSGGGGS (SEQ ID NO:372), and the like. The ordinarily skilled artisan will recognize that design of a peptide conjugated to any elements described above can include linkers that are all or partially flexible, such that the linker can include a flexible linker as well as one or more portions that confer less flexible structure.

### Combinations

In some embodiments, a polynucleotide provided by the replication incompetent recombinant retroviral particles has one or more transcriptional units that encode certain combinations of the one or more engineered signaling polypeptides. In some methods and compositions provided herein, modified and in illustrative embodiments genetically modified T cells include the combinations of the one or more engineered signaling polypeptides after transduction of T cells by the replication incompetent recombinant retroviral particles. It will be understood that the reference of a first polypeptide, a second polypeptide, a third polypeptide, etc. is for convenience and elements on a “first polypeptide” and those on a “second polypeptide” means that the elements are on different polypeptides that are referenced as first or second for reference and convention only, typically in further elements or steps to that specific polypeptide.

In some embodiments, the first engineered signaling polypeptide includes an extracellular antigen binding domain, which is capable of binding an antigen, and an intracellular signaling domain. In other embodiments, the first engineered signaling polypeptide also includes a T cell survival motif and/or a transmembrane domain In some embodiments, the first engineered signaling polypeptide does not include a co-stimulatory domain, while in other embodiments, the first engineered signaling polypeptide does include a co-stimulatory domain

In some embodiments, a second engineered signaling polypeptide includes a lymphoproliferative gene product and optionally an extracellular antigen binding domain In some embodiments, the second engineered signaling polypeptide also includes one or more of the following: a T cell survival motif, an intracellular signaling domain, and one or more co-stimulatory domains. In other embodiments, when two engineered signaling polypeptides are used, at least one is a CAR.

In one embodiment, the one or more engineered signaling polypeptides are expressed under a T cell specific promoter or a general promoter under the same transcript wherein in the transcript, nucleic acids encoding the engineered signaling polypeptides are separated by nucleic acids that encode one or more internal ribosomal entry sites (IREs) or one or more protease cleavage peptides.

In certain embodiments, the polynucleotide encodes two engineered signaling polypeptides wherein the first engineered signaling polypeptide includes a first extracellular antigen binding domain, which is capable of binding to a first antigen, and a first intracellular signaling domain but not a co-stimulatory domain, and the second polypeptide includes a second extracellular antigen binding domain, which is capable of binding VEGF, and a second intracellular signaling domain, such as for example, the signaling domain of a co-stimulatory molecule. In a certain embodiment, the first antigen is PSCA, PSMA, or BCMA. In a certain embodiment, the first extracellular antigen binding domain comprises an antibody or fragment thereof (e.g., scFv), e.g., an antibody or fragment thereof specific to PSCA, PSMA, or BCMA. In a certain embodiment, the second extracellular antigen binding domain that binds VEGF is a receptor for VEGF, i.e., VEGFR. In certain embodiments, the VEGFR is VEGFR1, VEGFR2, or VEGFR3. In a certain embodiment, the VEGFR is VEGFR2.

In certain embodiments, the polynucleotide encodes two engineered signaling polypeptides wherein the first engineered signaling polypeptide includes an extracellular tumor antigen binding domain and a CD3ζ signaling domain, and the second engineered signaling polypeptide includes an antigen-binding domain, wherein the antigen is an angiogenic or vasculogenic factor, and one or more co-stimulatory molecule signaling domains. The angiogenic factor can be, e.g., VEGF. The one or more co-stimulatory molecule signaling motifs can comprise, e.g., co-stimulatory signaling domains from each of CD27, CD28, OX40, ICOS, and 4-1BB.

In certain embodiments, the polynucleotide encodes two engineered signaling polypeptides wherein the first engineered signaling polypeptide includes an extracellular tumor antigen-binding domain and a CD3ζ signaling domain, the second polypeptide comprises an antigen-binding domain, which is capable of binding to VEGF, and co-stimulatory signaling domains from each of CD27, CD28, OX40, ICOS, and 4-1BB. In a further embodiment, the first signaling polypeptide or second signaling polypeptide also has a T cell survival motif. In some embodiments, the T cell survival motif is, or is derived from, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor or the TGFβ decoy receptor (TGF-β-dominant-negative receptor II (DNRII)).

In certain embodiments, the polynucleotide encodes two engineered signaling polypeptides wherein the first engineered signaling polypeptide includes an extracellular tumor antigen-binding domain and a CD3ζ signaling domain, and the second engineered signaling polypeptide includes an antigen-binding domain, which is capable of binding to VEGF, an IL-7 receptor intracellular T cell survival motif, and co-stimulatory signaling domains from each of CD27, CD28, OX40, ICOS, and 4-1BB.

In some embodiments, more than two signaling polypeptides are encoded by the polynucleotide. In certain embodiments, only one of the engineered signaling polypeptides includes an antigen binding domain that binds to a tumor-associated antigen or a tumor-specific antigen; each of the remainder of the engineered signaling polypeptides comprises an antigen binding domain that binds to an antigen that is not a tumor-associated antigen or a tumor-specific antigen. In other embodiments, two or more of the engineered signaling polypeptides include antigen binding domains that bind to one or more tumor-associated antigens or tumor-specific antigens, wherein at least one of the engineered signaling polypeptides comprises an antigen binding domain that does not bind to a tumor-associated antigen or a tumor-specific antigen.

In any of the aspects or embodiments herein that include an ASTR, the antigen can be a tumor-associated antigen or a tumor-specific antigen. In some embodiments, the tumor-associated antigen or tumor-specific antigen is Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD19, CD20, CD22, CD23, CD24, CD27, CD30, CD33, CD34, CD37, CD38, CD40, CD44, CD44v6, CD44v7/8, CD45, CD70, CD99, CD117, CD123, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), or PDL-1.

In some embodiments, the first engineered signaling polypeptide includes a first extracellular antigen binding domain that binds a first antigen, and a first intracellular signaling domain; and a second engineered signaling polypeptide includes a second extracellular antigen binding domain that binds a second antigen, or a receptor that binds the second antigen; and a second intracellular signaling domain, wherein the second engineered signaling polypeptide does not comprise a co-stimulatory domain In a certain embodiment, the first antigen-binding domain and the second antigen-binding domain are independently an antigen-binding portion of a receptor or an antigen-binding portion of an antibody. In a certain embodiment, either or both of the first antigen binding domain or the second antigen binding domain are scFv antibody fragments. In certain embodiments, the first engineered signaling polypeptide and/or the second engineered signaling polypeptide additionally comprises a transmembrane domain In a certain embodiment, the first engineered signaling polypeptide or the second engineered signaling polypeptide comprises a T cell survival motif, e.g., any of the T cell survival motifs described herein.

In another embodiment, the first engineered signaling polypeptide includes a first extracellular antigen binding domain that binds HER2 and the second engineered signaling polypeptide includes a second extracellular antigen binding domain that binds MUC-1.

In another embodiment, the second extracellular antigen binding domain of the second engineered signaling polypeptide binds an interleukin.

In another embodiment, the second extracellular antigen binding domain of the second engineered signaling polypeptide binds a damage associated molecular pattern molecule (DAMP; also known as an alarmin). In other embodiments, a DAMP is a heat shock protein, chromatin-associated protein high mobility group Box1 (HMGB1), S100A8 (also known as MRP8, or calgranulin A), S100A9 (also known as MRP14, or calgranulin B), serum amyloid A (SAA), deoxyribonucleic acid, adenosine triphosphate, uric acid, or heparin sulfate.

In certain embodiments, said second antigen is an antigen on an antibody that binds to an antigen presented by a tumor cell.

In some embodiments, signal transduction activation through the second engineered signaling polypeptide is non-antigenic, but is associated with hypoxia. In certain embodiments, hypoxia is induced by activation of hypoxia-inducible factor-1α (HIF-1α), HIF-β, HIF-2α, HIF-2β, HIF-3α, or HIF-3β.

In some embodiments, for example for modifying, genetically modifying, and/or transducing lymphocytes to be introduced or reintroduced by subcutaneous injection, expression of the one or more engineered signaling polypeptides is regulated by a control element, which is disclosed in more detail herein.

### Additional Sequences

The engineered signaling polypeptides, such as CARs, can further include one or more additional polypeptide domains, where such domains include, but are not limited to, a signal sequence; an epitope tag; an affinity domain; and a polypeptide whose presence or activity can be detected (detectable marker), for example by an antibody assay or because it is a polypeptide that produces a detectable signal. Non-limiting examples of additional domains for any of the aspects or embodiments provided herein, include a domain with at least 50%, 60%, 70%, 75%, 80%, 85%, 90%, 95%, 96%, 97%, 98%, 99% or 100% sequence identity to any of the following sequences as described below: a signal sequence, an epitope tag, an affinity domain, or a polypeptide that produces a detectable signal.

Signal sequences that are suitable for use in a subject CAR, e.g., in the first polypeptide of a subject CAR, include any eukaryotic signal sequence, including a naturally-occurring signal sequence, a synthetic (e g , man-made) signal sequence, etc. In some embodiments, for example, the signal sequence can be the CD8 signal sequence MALPVTALLLPLALLLHAARP (SEQ ID NO:72).

Suitable epitope tags include, but are not limited to, hemagglutinin (HA; e.g., YPYDVPDYA; SEQ ID NO:73); FLAG (e.g., DYKDDDDK; SEQ ID NO:74); c-myc (e.g., EQKLISEEDL; SEQ ID NO:75), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include HisS (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), hemagglutinin, e.g., HA Tag (YPYDVPDYA; SEQ ID NO:73), GST, thioredoxin, cellulose binding domain, RYIRS (SEQ ID NO:79), Phe-His-His-Thr (SEQ ID NO:80), chitin binding domain, S-peptide, T7 peptide, SH2 domain, C-end RNA tag, WEAAAREACCRECCARA (SEQ ID NO:81), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100proteins, parvalbumin, calbindin D9K, calbindin D28K, and calretinin, inteins, biotin, streptavidin, MyoD, Id, leucine zipper sequences, and maltose binding protein.

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

Safety Switch (Recognition and/or Elimination Domain)

Safety switches have been developed for use with cellular therapies to effect the reduction or elimination of infused cells in the case of adverse events. Any of the replication incompetent recombinant retroviral particles provided herein can include nucleic acids that encode a safety switch as part of, or separate from, nucleic acids encoding any of the engineered signaling polypeptides provided herein. Thus, any of the engineered signaling polypeptides provided herein, for example engineered signaling polypeptides in modified, genetically modified, and/or transduced lymphocytes to be introduced or reintroduced by subcutaneous injection, can include a safety switch. For example, any of the engineered T cells disclosed herein can include a safety switch.

Safety switch technologies can be broadly categorized into three groups based on their mechanism of action; metabolic (gene-directed enzyme prodrug therapy, GDEPT), dimerization induced apoptotic signals, and antibody mediated cytotoxicity.

In one aspect, the safety switch is a GDEPT. In some embodiments, the GDEPT can be a polynucleotide that encodes a viral thymidine kinase, such as that derived from the herpes simplex virus (HSV-TK). HSV-TK is a 376 amino acid protein with the sequence SEQ ID NO:368. In some embodiments, the GDEPT is a fragment of HSK-TV capable of converting the non-toxic drug ganciclovir (GCV) into GCV-triphosphate and leading to cell death by halting DNA replication. In other embodiments, the GDEPT can be a polynucleotide that encodes cytosine deaminase. Cytosine deaminase converts 5-fluorocytosine (5-FC) into the cytotoxic 5-fluorouracil (5-FU).

In one aspect the safety switch is based on dimerization induced apoptotic signals. In some embodiments, the safety switch is a chimeric protein comprised of an inducible dimerization domain linked in frame with components of an apoptotic pathway, such that conditional dimerization mediated by the binding of a cell-permeable chemical inducer of dimerization (CID) results in apoptosis of the cell. In some embodiments, the safety switch is inducible FAS (iFAS) comprised of one or more inducible dimerization domains fused to the cytoplasmic tail of the Fas receptor and localized to the membrane by a myristoyl group. In some embodiments, the safety switch is an inducible Caspase comprised of one or more inducible dimerization domains fused to a caspase, such as caspase-1 or caspase-9. In some embodiments the inducible dimerization domain is a cyclophilin and the CID is cyclosporin or a cyclosporin derivative. In some embodiments the inducible dimerization domain is a FKBP and the CID is an FK-506 dimer or derivative thereof, such as AP1903.

In one aspect the safety switch is based on antibody mediated cytotoxicity upon antibody binding to a recombinant polypeptide expressed on the cell surface (referred to herein as a cell tag). In some embodiments, the antibody binds to the cell tag and induces complement-dependent cytotoxicity (CDC) and/or antibody-dependent cell-mediated cytotoxicity (ADCC). In some embodiments, the cell tag is a myc or FLAG tag. In preferred embodiments, the cell tag polypeptide is non-immunogenic.

In some embodiments, the cell tag comprises an endogenous cell-surface molecule or a modified endogenous cell-surface molecule. The endogenous cell-surface molecule can be any cell-surface receptor, ligand, glycoprotein, cell adhesion molecule, antigen, integrin, or cluster of differentiation. Modifications to endogenous cell-surface molecules include modifications to the extracellular domain that reduce the ability of the cell-surface molecule to bind its cognate ligand or receptor, and/or modifications to the intracellular domain that reduce the natural signaling activity of the endogenous cell-surface molecule. Modifications to endogenous cell-surface molecules also include the removal of certain domains and/or the inclusion of domains from heterologous proteins or synthetic domains.

In some embodiments, the modified endogenous cell-surface molecule is a truncated tyrosine kinase receptor. In one aspect, the truncated tyrosine kinase receptor is a member of the epidermal growth factor receptor (EGFR) family (e.g., ErbB1 (HER1), ErbB2, ErbB3, and ErbB4), for example as disclosed in U.S. Pat. No. 8,802,374 or WO2018226897. In some embodiments, the cell tag can be a polypeptide that is recognized by an antibody that recognizes the extracellular domain of an EGFR member. In some embodiments, the cell tag can be at least 20 contiguous amino acids of an EGFR family member, or for example, between 20 and 50 contiguous amino acids of an EGFR family member. In some embodiments, a gene encoding an EGFR polypeptide including human epidermal growth factor receptor (EGFR) is constructed by removal of nucleic acid sequences that encode polypeptides including the membrane distal EGF-binding domain and the cytoplasmic signaling tail, but retaining the extracellular membrane proximal epitope recognized by an anti-EGFR antibody. For example, SEQ ID NO:82, is an exemplary polypeptide that is recognized by, and under the appropriate conditions bound by an antibody that recognizes the extracellular domain of an EGFR member. Such truncated EGFR polypeptides are sometimes referred to herein as eTags. In illustrative embodiments, eTags are recognized by monoclonal antibodies that are commercially available such as matuzumab, necitumumab panitumumab, and in illustrative embodiments, cetuximab. For example, eTAG was demonstrated to have suicide gene potential through Erbitux® mediated antibody dependent cellular cytotoxicity (ADCC) pathways. The inventors of the present disclosure have successfully expressed eTag in PBMCs using lentiviral vectors, and have found that expression of eTag in vitro by PBMCs exposed to Cetuximab, provided an effective elimination mechanism for PBMCs.

In some embodiments, the modified endogenous cell-surface molecule is a truncated version of a member of the TNF receptor superfamily. For example, a truncated version of the low affinity nerve growth factor receptor (LNGFR or TNFRSF16). Human LNGFR is a single pass type I transmembrane glycoprotein with the amino acid sequence of (SEQ ID NO:369) that comprises a 28 aa residue signal peptide, a 222 aa extracellular domain comprising 4 cysteine rich domains, a 22 aa transmembrane domain and a 155aa intracellular domain In some embodiments the cell-surface molecule comprises an epitope has at least 70%, 75%, 80%, 85%, 90%, 95%, 99%, or 100% identify to the amino acid sequence of the entire extracellular domain of LNGFR or to a truncated fragment of the extracellular domain such as residues 29-250, 65-250, or 108-250 of SEQ ID NO:369.

In some embodiments, the modified endogenous cell-surface molecule is a version of CD20. The human CD20 polypeptide is a multi-pass transmembrane protein encoded by a membrane-spanning 4-domains subfamily A member (MS4A1) gene with the amino acid sequence of SEQ ID NO:370. In some embodiments, CD20 comprises 4 transmembrane domain passes encompassing amino acids 57-78, 85-105, 121-141, and 189-209. In some embodiments, CD20 comprises 2 extracellular domains encompassing amino acids 79-84 and 142-188. In some embodiments, CD20 comprises 3 cytoplasmic domains encompassing amino acids 1-56, 106-120 and 210-297. In some embodiments, a CD20 polypeptide can be missing multiple domains or multiple portions of a domain relative to the wildtype polypeptide. In an embodiment, a CD20 polypeptide comprises M1-E263, M117-N214, M1-N214, V82-N214, or V82-I186 of endogenous CD20. In an embodiment, a CD20 polypeptide has at least 70%, 75%, 80%, 85%, 90%, 95%, 99%, or 100% identity to an amino acid sequence selected from K142-S185, P160-S185, or C167-C183 of SEQ ID NO:370. In illustrative embodiments, the truncated CD20 version comprises at least one copy of an epitope recognized by a monoclonal antibody such as ocrelizumab, obinutuzumab, ofatumumab, ibritumomab tiuxetan, tositumomab, ublituximab, and in further illustrative embodiments rituximab.

In some embodiments, the modified endogenous cell-surface molecule is a version of CD52. CD52 occurs endogenously in humans as a peptide of 12 amino acids linked at its C-terminus to a GPI anchor. In some embodiments, GPI can be used to anchor the polypeptide to the cell surface. In other embodiments, CD52 can be attached to the cell surface using a heterologous transmembrane domain In some embodiments, the truncated CD52 polypeptide can incorporate one or more epitopes recognized by an antibody such as HI186 (BioRad), YTH34.5 (BioRad), YTH66.9 (BioRad), or in illustrative embodiments, alemtuzumab. In some embodiments, the CD52 epitope has at least 70%, 75%, 80%, 85%, 90%, 95%, 99%, or 100% identify to the amino acid sequence of SEQ ID NO:371.

In some embodiments, the cell tag is itself an antibody that binds a predetermined binding partner antibody. In illustrative embodiments, the cell tag antibody is an anti-idiotypic antibody. In some embodiments, the anti-idiotypic antibody (Ab2) recognizes an epitope on the predetermined binding partner antibody (Ab1) that is distinct from the antigen binding site on Ab1. In illustrative embodiments, Ab2 binds the variable region of Ab1. In other illustrative embodiments, Ab2 binds the antigen-binding site of Ab 1. In certain embodiments, Ab2 may be from any animal including human and murine, or humanized or chimeric antibody or antibody derivative including antibody fragments (Fab, Fab′, F(ab′)2, scFv, diabodies, bispecific antibodies, and antibody fusion proteins. In certain embodiments, Ab2 is associated with the cell surface via its endogenous transmembrane domain In other embodiments, Ab2 is associated with the cell surface via a heterologous transmembrane domain or membrane attachment sequence such as GPI. In some embodiments, Ab1 is a commercially available monoclonal antibody. In illustrative embodiments, Ab1 is a commercially available monoclonal antibody therapeutic. In further illustrative embodiments, Ab1 is capable of mediating ADCC and/or CDC as described below. An example of a binding pair comprising an anti-idiotypic antibody (and methods of making the same) displayed on a cell line and cognate monoclonal Ab2 antibodies that mediate ADCC and CDC, is provided in WO2013188864.

In some embodiments, safety switches also function as flags that label or mark polynucleotides, polypeptides, or cells as being engineered. Such safety switches can be detected using standard laboratory techniques including PCR, Southern Blots, RT-PCR, Northern Blots, Western Blots, histology, and flow cytometry. For example, detection of eTAG by flow cytometry was used herein as an in vivo tracking marker for T cell engraftment in mice. In other embodiments, cell tags are used to enrich for engineered cells using antibodies or ligands optionally bound to a solid substrate such as a column or beads. For example, others have shown that application of biotinylated-cetuximab to immunomagnetic selection in combination with anti-biotin microbeads successfully enriches T cells that have been lentivirally transduced with eTAG containing constructs from as low as 2% of the population to greater than 90% purity without observable toxicity to the cell preparation.

In some embodiments, the safety switch is expressed as part of a single polynucleotide that also includes the CAR, or as part of a single polynucleotide that includes the lymphoproliferative element, or as a single polynucleotide that encodes both the CAR and the lymphoproliferative element. In some embodiments the polynucleotide encoding the safety switch is separated from the polynucleotide encoding the CAR and/or the polynucleotide encoding the lymphoproliferative element, by an internal ribosome entry site (IRES) or a ribosomal skip sequence and/or cleavage signal. The ribosomal skip and/or cleavage signal can be any ribosomal skip sequence and/or cleavage signal known in the art. The ribosomal skip sequence can be, for example, T2A with amino acid sequence GSGEGRGSLLTCGDVEENPGP (SEQ ID NO:83). Other examples of cleavage signals and ribosomal skip sequences include FMDV 2A (F2A); equine rhinitis A virus 2A (abbreviated as E2A); porcine teschovirus-1 2A (P2A); and Thoseaasigna virus 2A (T2A).

In some embodiments a safety switch, and in illustrative embodiments, a cell tag, is expressed as part of a fusion polypeptide, fused to a CAR. In other embodiments, a safety switch, and as exemplified empirically herein, a cell tag, is expressed fused to a lymphoproliferative element. Such constructs provide the advantage, especially in combination with other “space saving” elements provided herein, of taking up less genomic space on an RNA genome compared to separate polypeptides. In one illustrative embodiment, an eTag is expressed as a fusion polypeptide, fused the 5′ terminus of the c-Jun domain (SEQ ID NO:104), a transmembrane domain from CSF2RA (SEQ ID NO:129), a first intracellular domain from MPL (SEQ ID NO:283), and a second intracellular domain from CD40 (SEQ ID NO:208). When expressed as a polypeptide not fused to a CAR or lymphoproliferative element, the cell tag may be associated with the cell membrane via its natural membrane attachment sequence or via a heterologous membrane attachment sequence such as a GPI-anchor or transmembrane sequence. In illustrative embodiments cell tags are expressed on the T cell and/or NK cell but are not expressed on the replication incompetent recombinant retroviral particles. In some embodiments, polynucleotides, polypeptides, and cells comprise 2 or more safety switches.

### Chimeric Antigen Receptor

In some aspects of the present invention, an engineered signaling polypeptide is a chimeric antigen receptor (CAR) or a polynucleotide encoding a CAR, which, for simplicity, is referred to herein as “CAR.” A CAR of the present disclosure includes: a) at least one antigen-specific targeting region (ASTR); b) a transmembrane domain; and c) an intracellular activating domain In illustrative embodiments, the antigen-specific targeting region of the CAR is an scFv portion of an antibody to the target antigen. In illustrative embodiments, the intracellular activating domain is from CD3Z, CD3D, CD3E, CD3G, CD79A, CD79B, DAP12, FCER1G, FCGR2A, FCGR2C, DAP10/CD28, or ZAP70, and some further illustrative embodiments, from CD3z. In illustrative embodiments, the CAR further comprises a co-stimulatory domain, for example any of the co-stimulatory domains provided above in the Modulatory Domains section, and in further illustrative embodiments the co-stimulatory domain is the intracellular co-stimulatory domain of 4-1BB (CD137), CD28, ICOS, OX-40, BTLA, CD27, CD30, GITR, and HVEM. In some embodiments, the CAR includes any of the transmembrane domains listed in the Transmembrane Domain section above.

A CAR of the present disclosure can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell, an NK-T cell, and a macrophage. When present in the plasma membrane of a eukaryotic cell, a CAR of the present disclosure is active in the presence of one or more target antigens that, in certain conditions, binds the ASTR. The target antigen is the second member of the specific binding pair. The target antigen of the specific binding pair can be a soluble (e.g., not bound to a cell) factor; a factor present on the surface of a cell such as a target cell; a factor presented on a solid surface; a factor present in a lipid bilayer; and the like. Where the ASTR is an antibody, and the second member of the specific binding pair is an antigen, the antigen can be a soluble (e.g., not bound to a cell) antigen; an antigen present on the surface of a cell such as a target cell; an antigen presented on a solid surface; an antigen present in a lipid bilayer; and the like.

In some embodiments, the ASTR of a CAR is expressed as a separate polypeptide from the intracellular signaling domain In such embodiments, one or both of the polypeptides can include any of the transmembrane domains disclosed herein. In some embodiments, one or both of the polypeptides can include a heterologous signal sequence and/or a heterologous membrane attachment sequence. In some embodiments, the heterologous membrane attachment sequence is a GPI anchor attachment sequence.

In some instances, a CAR of the present disclosure, when present in the plasma membrane of a eukaryotic cell, and when activated by one or more target antigens, increases expression of at least one nucleic acid in the cell. For example, in some cases, a CAR of the present disclosure, when present in the plasma membrane of a eukaryotic cell, and when activated by the one or more target antigens, increases expression of at least one nucleic acid in the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the level of transcription of the nucleic acid in the absence of the one or more target antigens.

As an example, the CAR of the present disclosure can include an immunoreceptor tyrosine-based activation motif (ITAM)-containing intracellular signaling polypeptide.

A CAR of the present disclosure, when present in the plasma membrane of a eukaryotic cell, and when activated by one or more target antigens, can, in some instances, result in increased production of one or more cytokines by the cell. For example, a CAR of the present disclosure, when present in the plasma membrane of a eukaryotic cell, and when activated by the one or more target antigens, can increase production of a cytokine by the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine produced by the cell in the absence of the one or more target antigens. Cytokines whose production can be increased include, but are not limited to interferon gamma (IFN-γ), tumor necrosis factor-alpha (TNF-a), IL-2, IL-15, IL-12, IL-4, IL-5, IL-10; a chemokine; a growth factor; and the like.

In some embodiments, a CAR of the present disclosure, when present in the plasma membrane of a eukaryotic cell, and when activated by one or more target antigens, can result in both an increase in transcription of a nucleic acid in the cell and an increase in production of a cytokine by the cell.

In some instances, a CAR of the present disclosure, when present in the plasma membrane of a eukaryotic cell, and when activated by one or more target antigens, results in cytotoxic activity by the cell toward a target cell that expresses on its cell surface an antigen to which the antigen-binding domain of the first polypeptide of the CAR binds. For example, where the eukaryotic cell is a cytotoxic cell (e.g., an NK cell or a cytotoxic T lymphocyte), a CAR of the present disclosure, when present in the plasma membrane of the cell, and when activated by the one or more target antigens, increases cytotoxic activity of the cell toward a target cell that expresses on its cell surface the one or more target antigens. For example, where the eukaryotic cell is an NK cell or a T lymphocyte, a CAR of the present disclosure, when present in the plasma membrane of the cell, and when activated by the one or more target antigens, increases cytotoxic activity of the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cell in the absence of the one or more target antigens.

In some embodiments, a CAR of the present disclosure, when present in the plasma membrane of a eukaryotic cell, and when activated by one or more target antigens, can result in other CAR activation related events such as proliferation and expansion (either due to increased cellular division or anti-apoptotic responses).

In some embodiments, a CAR of the present disclosure, when present in the plasma membrane of a eukaryotic cell, and when activated by one or more target antigens, can result in other CAR activation related events such as intracellular signaling modulation, cellular differentiation, or cell death.

In some embodiments, CARs of the present disclosure are microenvironment restricted. This property is typically the result of the microenvironment restricted nature of the ASTR domain of the CAR. Thus, CARs of the present disclosure can have a lower binding affinity or, in illustrative embodiments, can have a higher binding affinity to one or more target antigens under a condition(s) in a microenvironment than under a condition in a normal physiological environment.

In certain illustrative embodiments, CARs provided herein comprise a co-stimulatory domain in addition to an intracellular activating domain, wherein the co-stimulatory domain is any of the intracellular signaling domains provided herein for lymphoproliferative elements (LEs), such as, for example, intracellular domains of CLEs. In certain illustrative embodiments, the co-stimulatory domains of CARs herein are first intracellular domains (P3 domains) identified herein for CLEs or P4 domains that are shown as effective intracellular signaling domains of CLEs herein in the absence of a P3 domain Furthermore, in certain illustrative embodiments, co-stimulatory domains of CARs can comprise both a P3 and a P4 intracellular signaling domain identified herein for CLEs. Certain illustrative subembodiments include especially effective P3 and P4 partner intracellular signaling domains as identified herein for CLEs. In illustrative embodiments, the co-stimulatory domain is other than an ITAM-containing intracellular domain of a CAR either as part of the co-stimulatory domain, or in further illustrative embodiments as the only co-stimulatory domain

In these embodiments that include a CAR with a co-stimulatory domain identified herein as an effective intracellular domain of an LE, the co-stimulatory domain of a CAR can be any intracellular signaling domain in Table 1 provided herein. Active fragments of any of the intracellular domains in Table 1 can be a co-stimulatory domain of a CAR. In illustrative embodiments, the ASTR of the CAR comprises an scFV. In illustrative embodiments, in addition to the c-stimulatory intracellular domain of a CLE, these CARs comprise an intracellular activating domain that in illustrative embodiments is a CD3Z, CD3D, CD3E, CD3G, CD79A, CD79B, DAP12, FCER1G, FCGR2A, FCGR2C. DAP10/CD28, or ZAP70 intracellular activating domain, or in further illustrative embodiments is a CD3z intracellular activating domain

In these illustrative embodiments, the co-stimulatory domain of a CAR can comprise an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL7RA, IL9R, IL10RA, IL10RB, IL11RA, IL12RB1, IL12RB2, IL13RA1, IL13RA2, IL15RA, IL17RB, IL17RC, IL17RD, IL18R1, IL18RAP, IL20RA, IL20RB, IL21R, IL22RA1, IL23R, IL27RA, IL31RA, LEPR, LIFR, LMP1, MPL, MyD88, OSMR, or PRLR. In some embodiments, the co-stimulatory domain of a CAR can include an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL9R, IL10RA, IL10RB, IL11RA, IL13RA1, IL13RA2, IL17RB, IL17RC, IL17RD, IL18R1, IL18RAP, IL20RA, IL20RB, IL22RA1, IL31RA, LEPR, LIFR, LMP1, MPL, MyD88, OSMR, or PRLR. In some embodiments, the co-stimulatory domain of a CAR can include an intracellular domain or a functional fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL12RB1, IL12RB2, IL13RA2, IL15RA, IL17RD, IL21R, IL23R, IL27RA, IL31RA, LEPR, MPL, MyD88, or OSMR. In some embodiments, the co-stimulatory domain of a CAR can include an intracellular domain or a fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL13RA2, IL17RD, IL31RA, LEPR, MPL, MyD88, or OSMR. In some embodiments, the co-stimulatory domain of a CAR can include an intracellular domain or a functional signaling fragment thereof that includes a signaling domain from CSF2RB, CSF3R, IFNAR1, IFNGR1, IL2RB, IL2RG, IL6ST, IL10RA, IL12RB2, IL17RC, IL17RE, IL18R1, IL27RA, IL31RA, MPL, MyD88, OSMR, or PRLR. In some embodiments, the co-stimulatory domain of a CAR can include an intracellular domain or a functional signaling fragment thereof that includes a signaling domain from CSF2RB, CSF3R, IFNGR1, IL2RB, IL2RG, IL6ST, IL10RA, IL17RE, IL31RA, MPL, or MyD88.

In some embodiments, the co-stimulatory domain of a CAR can include an intracellular domain or a fragment thereof that includes a signaling domain from CSF3R, IL6ST, IL27RA, MPL, and MyD88. In certain illustrative subembodiments, the intracellular activating domain of the CAR is derived from CD3z.

### Recombinant T Cell Receptors (TCRs)

T Cell Receptors (TCRs) recognize specific protein fragments derived from intracellular as well as extracellular proteins. When proteins are broken into peptide fragments, they are presented on the cell surface with another protein called major histocompatibility complex, or MHC, which is called the HLA (human leukocyte antigen) complex in humans. Three different T cell antigen receptors combinations in vertebrates are αβTCR, γδTCR and pre-TCR. Such combinations are formed by dimerization between members of dimerizing subtypes, such as an α TCR subunit and a β TCR subunit, a γ TCR subunit and a δ TCR subunit, and for pre-TCRs, a pTα subunit and a β TCR subunit. A set of TCR subunits dimerize and recognize a target peptide fragment presented in the context of an MHC. The pre-TCR is expressed only on the surface of immature αβT cells while the αβ TCR is expressed on the surface of mature αβT cells and NK T cells, and γδTCR is expressed on the surface of γδT cells. αβTCRs on the surface of a T cell recognize the peptide presented by MHCI or MHCII and the αβ TCR on the surface of NK T cells recognize lipid antigens presented by CD1. γδTCRs can recognize MHC and MHC-like molecules, and can also recognize non-MHC molecules such as viral glycoproteins. Upon ligand recognition, αβTCRs and γδTCRs transmit activation signals through the CD3zeta chain that stimulate T cell proliferation and cytokine secretion.

TCR molecules belong to the immunoglobulin superfamily with its antigen-specific presence in the V region, where CDR3 has more variability than CDR1 and CDR2, directly determining the antigen binding specificity of the TCR. When the MHC-antigen peptide complex is recognized by a TCR, the CDR1 and CDR2 recognize and bind the sidewall of the MHC molecule antigen binding channel, and the CDR3 binds directly to the antigenic peptide. Recombinant TCRs may thus be engineered that recognize a tumor-specific protein fragment presented on MHC.

Recombinant TCR's such as those derived from human TCRα and TCRβ pairs that recognize specific peptides with common HLAs can thus be generated with specificity to a tumor specific protein (Schmitt, TM et al., 2009). The target of recombinant TCRs may be peptides derived from any of the antigen targets for CAR ASTRs provided herein, but are more commonly derived from intracellular tumor specific proteins such as oncofetal antigens, or mutated variants of normal intracellular proteins or other cancer specific neoepitopes. Libraries of TCR subunits may be screened for their selectivity to a target antigen. Screens of natural and/or recombinant TCR subunits can identify sets of TCR subunits with high avidities and/or reactivities towards a target antigen. Members of such sets of TCR subunits can be selected and cloned to produce one or more polynucleotide encoding the TCR subunit.

Polynucleotides encoding such a set of TCR subunits can be included in a replication incompetent recombinant retroviral particle to genetically modify a lymphocyte, or in illustrative embodiments, a T cell or an NK cell, such that the lymphocyte expresses the recombinant TCR. Accordingly, in any aspect or embodiment provided herein that includes a polynucleotide encoding a CAR or an engineered signaling polypeptide that is a CAR, the CAR can be replaced by a set of γδTCR chains, or in illustrative embodiments αβTCR chains. TCR chains that form a set may be co-expressed using a number of different techniques to co-express the two TCR chains as is disclosed herein for expressing two or more other engineered signaling polypeptides such as CARs and lymphoproliferative elements. For example, protease cleavage epitopes such as 2A protease, internal ribosomal entry sites (IRES), and separate promoters may be used.

Several strategies have been employed to reduce the likelihood of mixed TCR dimer formation. In general, this involves modification of the constant (C) domains of the TCRα and TCRβ chains to promote the preferential pairing of the introduced TCR chains with each other, while rendering them less likely to successfully pair with endogenous TCR chains. One approach that has shown some promise in vitro involves replacement of the C domain of human TCRα and TCRβ chains with their mouse counterparts. Another approach involves mutation of the human TCRα common domain and TCRβ chain common regions to promote self-pairing, or the expression of an endogenous TCR alpha and TCR beta miRNA within the viral gene construct. Accordingly, in some embodiments provided herein that include one or more sets of TCR chains as engineered signaling polypeptides, each member of the set of TCR chains, in illustrative embodiments αβTCR chains, comprises a modified constant domain that promotes preferential pairing with each other. In some subembodiments, each member of a set of TCR chains, in illustrative embodiments αβTCR chains, comprises a mouse constant domain from the same TCR chain type, or a constant domain from the same TCR chain subtype with enough sequences derived from a mouse constant domain from the same TCR chain subtype, such that dimerization of the set of TCR chains to each other is preferred over, or occurs to the exclusion of, dimerization with human TCR chains. In other subembodiments, each member of a set of TCR chains, in illustrative embodiments αβTCR chains, comprises corresponding mutations in its constant domain, such that dimerization of the set of TCR chains to each other is preferred over, or occurs to the exclusion of, dimerization with TCR chains that have human constant domains. Such preferred or exclusive dimerization in illustrative embodiments, is under physiological conditions.

In some embodiments provided herein that include one or more sets of TCR chains as engineered signaling polypeptides, the constant regions of the members of each of the one or more sets of TCR chains are swapped. Thus, the α TCR subunit of the set has a β TCR constant region, and the β TCR subunit of the set has a α TCR constant region. Not to be limited by theory, it is believed that such swapping may prevent mispairing with endogenous counterparts.

### Lymphoproliferative Elements

Many of the embodiments provided herein include a lymphoproliferative element, or a nucleic acid encoding the same, typically as part of an engineered signaling polypeptide. Accordingly, in some aspects of the present invention, for example for modified and/or genetically modified lymphocytes to be introduced or reintroduced by subcutaneous injection, an engineered signaling polypeptide is a lymphoproliferative element (LE) such as a chimeric lymphoproliferative element (CLE). Typically, the LE comprises an extracellular domain, a transmembrane domain, and at least one intracellular signaling domain that drives proliferation, and in illustrative embodiments a second intracellular signaling domain.

The extracellular domains, transmembrane domains, and intracellular domains of LEs can vary in their respective amino acid lengths. For example, for embodiments that include a replication incompetent retroviral particle, there are limits to the length of a polynucleotide that can be packaged into a retroviral particle so LEs with shorter amino acid sequences can be advantageous in certain illustrative embodiments. In some embodiments, the overall length of the LE can be between 3 and 4000 amino acids, for example between 10 and 3000, 10 and 2000, 50 and 2000, 250 and 2000 amino acids, and, in illustrative embodiments between 50 and 1000, 100 and 1000 or 250 and 1000 amino acids. The extracellular domain, when present to form an extracellular and transmembrane domain, can be between 1 and 1000 amino acids, and is typically between 4 and 400, between 4 and 200, between 4 and 100, between 4 and 50, between 4 and 25, or between 4 and 20 amino acids. In one embodiment, the extracellular region is GGGS for an extracellular and transmembrane domain of this aspect of the invention. The transmembrane domains, or transmembrane regions of extracellular and transmembrane domains, can be between 10 and 250 amino acids, and are more typically at least 15 amino acids in length, and can be, for example, between 15 and 100, 15 and 75, 15 and 50, 15 and 40, or 15 and 30 amino acids in length. The intracellular signaling domains can be, for example, between 10 and 1000, 10 and 750, 10 and 500, 10 and 250, or 10 and 100 amino acids. In illustrative embodiments, the intracellular signaling domain can be at least 30, or between 30 and 500, 30 and 250, 30 and 150, 30 and 100, 50 and 500, 50 and 250, 50 and 150, or 50 and 100 amino acids. In some embodiments, an intracellular signaling domain for a particular gene is at least 50%, 60%, 70%, 75%, 80%, 85%, 90%, 95%, 98%, 99% or 100% identical to at least 10, 25, 30, 40, 50, or all the amino acids from a sequence of that intracellular signaling domain, such as a sequence provided herein for that intracellular domain, up to the size of the entire intracellular domain sequence, and can include for example, up to an additional 1, 2, 3, 4, 5, 10, 20, or 25 amino acids, provided that such sequence still is capable of providing any of the properties of LEs disclosed herein.

In some embodiments, the lymphoproliferative element can include a first and/or second intracellular signaling domain In some embodiments, the first and/or second intracellular signaling domain can include CD2, CD3D, CD3E, CD3G, CD4, CD8A, CD8B, CD27, mutated Delta Lck CD28, CD28, CD40, CD79A, CD79B, CRLF2, CSF2RB, CSF2RA, CSF3R, EPOR, FCER1G, FCGR2C, FCGRA2, GHR, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, LEPR, LIFR, LMP1, MPL, MYD88, OSMR, PRLR, TNFRSF4, TNFRSF8, TNFRSF9, TNFRSF14, or TNFRSF18, or functional mutants and/or fragments thereof. In illustrative embodiments, the first intracellular signaling domain can include MyD88, or a functional mutant and/or fragment thereof. In further illustrative embodiments, the first intracellular signaling domain can include MyD88, or a functional mutant and/or fragment thereof, and the second intracellular signaling domain can include ICOS, TNFRSF4, or TNSFR18, or functional mutants and/or fragments thereof. In some embodiments, the first intracellular domain is MyD88 and the second intracellular domain is an ITAM-containing intracellular domain, for example, an intracellular domain from CD3Z, CD3D, CD3E, CD3G, CD79A, CD79B, DAP12, FCER1G, FCGR2A, FCGR2C, DAP10/CD28, or ZAP70. In some embodiments, the second intracellular signaling domain can include TNFRSF18, or a functional mutant and/or fragment thereof.

In some embodiments, the lymphoproliferative element can include a fusion of an extracellular domain and a transmembrane domain In some embodiments, the fusion of an extracellular domain and a transmembrane domain can include eTAG IL7RA Ins PPCL (interleukin 7 receptor), Myc LMP1, LMP1, eTAG CRLF2, eTAG CSF2RB, eTAG CSF3R, eTAG EPOR, eTAG GHR, eTAG truncated after Fn F523C IL27RA, or eTAG truncated after Fn S505N MPL, or functional mutants and/or fragments thereof. In some embodiments, the lymphoproliferative element can include an extracellular domain. In some embodiments, the extracellular domain can include cell tag with 0, 1, 2, 3, or 4 additional alanines at the carboxy terminus. In some embodiments, the extracellular domain can include Myc or an eTAG with 0, 1, 2, 3, or 4 additional alanines at the carboxy terminus, or functional mutants and/or fragments thereof. For any embodiment of a lymphoproliferative element disclosed herein that includes a cell tag, there is a corresponding embodiment that is identical but lacks the cell tag and optionally lacks any linker sequence that connected the cell tag to the lymphoproliferative element.

In some embodiments, the lymphoproliferative element can include a transmembrane domain In some embodiments, the transmembrane domain can include a transmembrane domain from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof.

CLEs for use in any aspect or embodiment herein can include any CLE disclosed in WO2019/055946 (incorporated by reference herein, in its entirety), the vast majority of which were designed to be and are believed to be constitutively active typically because they constitutively activate a signaling pathway. In some embodiments, the constitutively active signaling pathways include activation of Jak/Stat pathways including Jak1, Jak2, Jak3, and Tyk2 and STATs such as STAT1, STAT2, STAT3, STAT4, STAT5, STAT6, and in illustrative embodiments, STAT3 and/or STAT5. In some embodiments, a CLE includes one or more STAT-activation domains. In some embodiments, a CLE includes two or more, three or more, four or more, five or more, or six or more STAT-activation domains. In some embodiments, at least one of the one or more STAT-activation domains is, or is derived from BLNK, IL2RG, EGFR, EpoR, GHR, IFNAR1, IFNAR2, IFNAR1/2, IFNLR1, IL10R1, IL12Rb1, IL12Rb2, IL21R, IL2Rb, IL2small, IL7R, IL7Ra, IL9R, IL15R, and IL21R, as are known in the art. In some embodiments, two or more STAT-activation domains are, or are derived from two or more different receptors. In some embodiments, the constitutively active signaling pathways include activation of a TRAF pathway through activation of TNF receptor associated factors such as TRAF3, TRAF4, TRAF7, and in illustrative embodiments TRAF1, TRAF2, TRAF5, and/or TRAF6. Thus, in certain embodiments, lymphoproliferative elements for use in any of the kits, methods, uses, or compositions herein, are constitutively active and comprise an intracellular signaling domain that activates a Jak/Stat pathway and/or a TRAF pathway. In some embodiments, the constitutively active signaling pathways include activation of PI3K pathways. In some embodiments, the constitutively active signaling pathways include activation of PLC pathways. Thus, in certain embodiments, lymphoproliferative elements for use in any of the kits, methods, uses, or compositions herein, are constitutively active and comprise an intracellular signaling domain that activates a Jak/Stat pathway a TRAF pathway, a PI3K pathway, and/or a PLC pathway. As illustrated therein, where there is a first and a second intracellular signaling domain of a CLE, the first intracellular signaling domain is positioned between the membrane associating motif, for example, a transmembrane domain, and the second intracellular domain

In some embodiments, the lymphoproliferative elements provided herein include one or more, or all of the binding domains, including those disclosed herein, responsible for signaling found in the corresponding lymphoproliferative element in nature. In some embodiments, the lymphoproliferative elements provided herein include one or more JAK binding domains. In some embodiments, the JAK-binding domain is, or is derived from, EPOR, GP130, PRLR, GHR, GCSFR, or TPOR/MPLR. JAK-binding domains from these proteins are known in the art and a skilled artisan will understand how to use them. For example, residues 273-338 of EpoR and residues 478-582 of TpoR are known to be JAK-binding domains. Conserved motifs that are found in intracellular domains of cytokine receptors that are responsible for this signaling are known and are present in certain illustrative lymphoproliferative elements provided herein (see e.g., Morris et al., “The molecular details of cytokine signaling via the JAK/STAT pathway,” Protein Science (2018) 27:1984-2009). The Box1 and Box2 motifs are involved in binding to JAKs and signal transduction, although the Box2 motif presence is not always required for a proliferative signal (Murakami et al. Proc Natl Acad Sci USA. 1991 Dec. 15; 88(24):11349-53; Fukunaga et al. EMBO J. 1991 October; 10(10):2855-65; and O'Neal and Lee. Lymphokine Cytokine Res. 1993 October; 12(5):309-12). Accordingly, in some embodiments a lymphoproliferative element herein is a transgenic Box1-containing cytokine receptor that includes an intracellular domain of a cytokine receptor comprising a Box1 Janus kinase (JAK)-binding motif, optionally a Box2 JAK-binding motif, and a Signal Transducer and Activator of Transcription (STAT) binding motif comprising a tyrosine residue. In some embodiments, a lymphoproliferative element includes two or more JAK-binding motifs, for example three or more or four or more JAK-binding motifs, which in illustrative are the binding motifs found in natural versions of the corresponding lymphoproliferative element.

Intracellular domains from IFNAR1, IFNGR1, IFNLR1, IL2RB, IL4R, IL5RB, IL6R, IL6ST, IL7RA, IL9R, IL10RA, IL21R, IL27R, IL31RA, LIFR, and OSMR are known in the art to activate JAK1 signaling and thus comprise a JAK1 binding motif. Intracellular domains from CRLF2, CSF2RA, CSF2RB, CSF3R, EPOR, GHR, IFNGR2, IL3RA, IL5RA, IL6ST, IL20RA, IL20RB, IL23R, IL27R, LEPR, MPL, and PRLR are known in the art to activate JAK2 and thus comprise a JAK2 binding motif. Intracellular domains from IL2RG are known in the art to activate JAK3 and thus comprise a JAK3 binding motif. Intracellular domains from GHR, IFNAR1, IFNAR2, IFNGR1, IFNGR2, IL2RB, IL2RG, IL4R, IL5RA, IL5RB, IL7RA, IL9R, IL21R, IL22RA1, IL31RA, LIFR, MPL, and OSMR are known in the art to activate STAT1. Intracellular domains from IFNAR1 and IFNAR2 are known in the art to activate STAT2. Intracellular domains from GHR, IL2RB, IL2RG, IL6R, IL7RA, IL9R, IL10RA, IL10RB, IL21R, IL22RA1, IL23R, IL27R, IL31RA, LEPR, LIFR, MPL, and OSMR are known in the art to activate STAT3. Intracellular domains from IL12RB1 are known in the art to activate STAT4. Intracellular domains from CSF2RA, CSF2RB, CSF3R, EPOR, GHR, IL2RB, IL2RG, IL3RA, IL4R, IL5RA, IL5RB, IL7RA, IL9R, IL15RA, IL20RA, IL20RB, IL21R, IL22RA1, IL31RA, LIFR, MPL, OSMR, and PRLR are known in the art to activate STAT5. Intracellular domains from IL4R and OSMR are known in the art to activate STAT6. The genes and intracellular domains thereof that are found in a first intracellular domain are the same as the optional second intracellular domain, except that if the first and second intracellular domain are identical, then at least one, and typically both the transmembrane domain and the extracellular domain are not from the same gene.

In some embodiments, a lymphoproliferative element herein can include one or more intracellular signaling domains that include one or more Box1 motifs. In some embodiments, the one or more intracellular signaling domains that include one or more Box1 motifs can be IL7RA (Box1 motif at residues 9-17 of SEQ ID NOs:248 and 249), IL12RB ((Box1 motifs at residues 10-12 of SEQ ID NOs:254 and 255; and residues 107-110 and 139-142 of SEQ ID NO:256), IL31RA (Box1 motifs at residues 12-15 of SEQ ID NOs:275 and 276), CSF2RB (Box1 motif at residues 14-22 of SEQ ID NO:213), IL2RB (Box1 motif at residues 13-21 of SEQ ID NO:240), IL6ST (Box1 motif at residues 10-18 of SEQ ID NO:247), IL2RG (Box1 motif at residues 3-11 of SEQ ID NO:241), IL27RA (Box1 motif at residues 17-25 of SEQ ID NO:273), MPL (Box1 motif at residues 17-20 of SEQ ID NO:283), OSMR (Box1 motif at residues 16-30 of SEQ ID NO:294), IFNAR2 (Box1 motif at residues 23-31 of SEQ ID NO:227), CSF3R, or EPOR (Box1 motif at residues 257-264 of full-length EPOR).

In some embodiments, a lymphoproliferative element herein can include one or more intracellular signaling domains that include one or more Box2 motifs. In some embodiments, the one or more intracellular signaling domains that include one or more Box2 motifs can be MPL (Box2 motif at residues 46-64 in SEQ ID NO:283), IFNAR2 (Box1 motif at residues 37-46 of SEQ ID NO:227), CSF3R, or EPOR (Box2 motif at residues 303-313 of full-length EPOR). EPOR also contains an extended Box2 motif (residues 329-372 of full-length EPOR) important for binding tyrosine kinase receptor KIT, which, in some embodiments, a lymphoproliferative element can include. CSF3R also contains a Box3 motif, which, in some embodiments, a lymphoproliferative element can include.

Some intracellular signaling domains have hydrophobic residues at positions −1, −2, and −6 relative to the Box1 motif, that form a “switch motif,” which is required for cytokine-induced JAK2 activation but not for JAK2 binding (Constantinescu et al. Mol Cell. 2001 February; 7(2):377-85; and Huang et al. Mol Cell. 2001 December; 8(6):1327-38). Accordingly, in certain embodiments, the Box1 motif-containing lymphoproliferative element has a switch motif, which in illustrative embodiments has one or more, and preferably all hydrophobic residues at positions -1, -2, and -6 relative to the Box1 motif. In certain embodiments, the Box1 motif an ICD of a lymphoproliferative element is located proximal to the transmembrane (TM) domain (for example between 5 and 15 or about 10 residues downstream from the TM domain) relative to the Box2 motif, which is located proximal to the transmembrane domain (for example between 10 and 50 residues downstream from the TM domain) relative to the STAT binding motif. The STAT binding motif typically comprising a tyrosine residue, the phosphorylation of which affects binding of a STAT to the STAT binding motif of the lymphoproliferative element. In some embodiments, the ICDs comprising multiple STAT binding motifs where multiple STAT binding motifs are present in a native ICD (e.g., EPO receptor and IL-6 receptor signaling chain (gp130). In some embodiments, the switch motif containing intracellular signaling domain can be MPL (switch motif at residues 11, 15, and 16 of SEQ ID NO:283).

In some embodiments, a lymphoproliferative element herein can include one or more intracellular signaling domains that include one or more phosphorylatable residues, for example, a phosphorylatable serine, threonine, or tyrosine. In some embodiments, the one or more intracellular signaling domains that include one or more phosphorylatable residues can be IL31RA (phosphorylatable tyrosines at residues Y96, Y237, and Y165 of SEQ ID NO:275; not present in SEQ ID NO:276), CD27 (phosphorylatable serine at residue S6 of SEQ ID NO:205), CSF2RB (phosphorylatable tyrosine at residue Y306 of SEQ ID NO:213), IL6ST (phosphorylatable serines at residues S20, S26, S141, S148, S188, and S198 of SEQ ID NO:247), MPL (phosphorylatable tyrosines at residues Y8, Y29, Y78, Y113, and Y118 of SEQ ID NO: 283), CD79B (phosphorylatable tyrosines at residues Y16 and Y27 of SEQ ID NO: 211), OSMR (phosphorylatable serines at residues S65 and S128 of SEQ ID NO:294), or CD3G (phosphorylatable serines at residues S123 and S126 of full-length CD3G). In some embodiments, a lymphoproliferative element that includes a CSF3R intracellular domain can include one, two, three, or all of the tyrosine residues corresponding to Y704, Y729, Y744, and Y764 of full-length CSF3R, various combinations of which have been shown to be important for binding Stat3, SOCS3, Grb2, and p21Ras. In some embodiments, a lymphoproliferative element herein can include one or more intracellular signaling domains that has one or more of its phosphorylatable residues mutated to a phosphomimetic residue, for example, aspartic acid or glutamic acid. In some embodiments, a lymphoproliferative element herein can include one or more intracellular signaling domains that has one or more of its phosphorylatable tyrosines mutated to a non-phosphorylatable residue, for example, alanine, valine, or phenylalanine. In some embodiments, a lymphoproliferative element that includes a CSF3R intracellular domain can include one or more mutations corresponding to T615A and T618I of full-length CSF3R, which have been shown to increase receptor dimerization and activity.

In some embodiments, a lymphoproliferative element herein can include one or more intracellular signaling domains that include one or more ubiquitination targeting motif residues. In some embodiments, the one or more intracellular signaling domains that include one or more ubiquitination targeting motif residues can be MPL (residues at K40 and K60 of SEQ ID NO:283) or OX40 (residues at K17 and K41 of SEQ ID NO:296). In some embodiments herein, an intracellular domain including ubiquitination targeting motif residues can have one or more of the lysines mutated to arginine or another amino acid.

In some embodiments, a lymphoproliferative element herein can include one or more intracellular signaling domains that include one or more TRAF binding sites. Not to be limited by theory, TRAF1, TRAF2, and TRAF3 binding sites include the amino acid sequence PXQXT (SEQ ID NO:303), where each X can be any amino acid, a distinct TRAF2 binding site includes the consensus sequence SXXE (SEQ ID NO:304) where each X can be any amino acid, and a TRAF6 binding site includes the consensus sequence QXPXEX (SEQ ID NO:305). In some embodiments, the one or more intracellular signaling domains that include one or more TRAF binding sites can be CD40 (binding sites for TRAF1, TRAF2, and TRAF3 at residues 35-39 of SEQ ID NO:208; TRAF2 binding site at residues 57-60 of SEQ ID NO:208; TRAF6 binding site at residues 16-21 of SEQ ID NO:208), or OX40 (TRAF1, TRAF2, TRAF3, and TRAF5 binding motif at residues 20-27 of SEQ ID NO:296).

In some embodiments, a lymphoproliferative element herein can include one or more intracellular signaling domains that include a TIR domain. In some embodiments, the one or more intracellular signaling domains that include a TIR domains can be IL17RE (TIR domain at residues 13-136 of SEQ ID NO:265), IL18R1 (TIR domain at residues 28-170 of SEQ ID NO:266), or MyD88 (TIR domain at residues 160-304 of SEQ ID NO:284).

In some embodiments, a lymphoproliferative element herein can include one or more intracellular signaling domains that include a PI3K binding motif domain In some embodiments, the one or more intracellular signaling domains that include a PI3K binding motif can be CD28 (PI3K binding motifs at residues 12-15 of SEQ ID NOs:206 and 207, which also binds Grb2), ICOS (PI3K binding motif at residues 19-22 of SEQ ID NO:225, which can be mutated F21Q to increase IL-2 production and/or to bind Grb2), OX40 (p85 PI3K binding motif at residues 34-57 of full-length OX40)

In some embodiments, a lymphoproliferative element herein can include one or more intracellular signaling domains that include a dileucine motif. In some embodiments, the one or more intracellular signaling domains that include a dileucine motif can be IFNGR2 (dileucine motif at residues 8-9 of SEQ ID NO:230) or CD3G (dileucine motif at residues 131-132 of full-length CD3G). In some embodiments, one or both of the residues in the dileucine motif can be mutated.

In some embodiments, a lymphoproliferative element herein can include one or more intracellular signaling domains that include one or more N-terminal death domains. In some embodiments, the one or more intracellular signaling domains that include one or more N-terminal death domains can be MyD88 (N-terminal death domain at residues 29-106 of SEQ ID NO:284) or a TNFR. The cytoplasmic domains of TNF receptors (TNFRs), which in illustrative embodiments can be TNFRSF4, TNFRSF8, TNFRSF9, TNFRSF14, or TNFRSF18, can recruit signaling molecules, including TRAFs (TNF receptor-associated factors) and/or “death domain” (DD) molecules. The domains, motifs, and point mutations of TNFRs that induce proliferation and/or survival of T cells and/or NK cells are known in the art and a skilled artisan can identify corresponding domains, motifs, and point mutations in TNFR polypeptides. A skilled artisan will be able to identify the TRAF- and/or DD-binding motif in the different TNFR families using, for example, sequence alignments to known binding motifs. In some embodiments, a lymphoproliferative element that includes a TNFR intracellular domain can include one or more TRAF-binding motifs. In some embodiments, a lymphoproliferative element that includes a TNFR intracellular domain does not include a DD-binding motif, or has one or more DD-binding motifs deleted or mutated within the intracellular domain. In some embodiments, a lymphoproliferative element that includes a TNFR intracellular domain can recruit TRADD and/or TRAF2. TNFRs also include cysteine-rich domains (CRDs) that are important for ligand binding (Locksley RM et al. Cell. 2001 Feb. 23;104(4):487-501). In some embodiments, a lymphoproliferative element that includes a TNFR intracellular domain does not include a TNFR CRD.

In some embodiments, a lymphoproliferative element herein can include one or more intracellular signaling domains that include one or more intermediate domains that interact with IL-1R associated kinase. In some embodiments, the one or more intracellular signaling domains that include one or more intermediate domains can be MyD88 (intermediate domain at residues 107-156 of SEQ ID NO:284),

In some embodiments, a lymphoproliferative element that includes an intracellular domain from IL7RA can include one or more of the S region or T region (S region at residues 359-394 and T region at residues Y401, Y449, and Y456 of full-length IL7RA). In illustrative embodiments of lymphoproliferative elements that include a first intracellular domain derived from IL7RA, the second intracellular domain can be derived from TNFRSF8.

In illustrative embodiments of lymphoproliferative elements that include a first intracellular domain derived from CD40, the second intracellular domain can be other than an intracellular domain derived from MyD88, a CD28 family member (e.g., CD28, ICOS), Pattern Recognition Receptor, a C-reactive protein receptor (i.e., Nodi, Nod2, PtX3-R), a TNF receptor, CD40, RANK/TRANCE-R, OX40, 4-1BB), an HSP receptor (Lox-1 and CD91), or CD28. Pattern Recognition Receptors include, but are not limited to endocytic pattern-recognition receptors (i.e., mannose receptors, scavenger receptors (i.e., Mac-1, LRP, peptidoglycan, teichoic acids, toxins, CD1 1 c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), and RIG1.

In some embodiments, a lymphoproliferative element that includes an intracellular domain from MyD88 can include one or more of the mutations L93P, R193C, and L265P in full-length MyD88 (mutations L93P, R196C, and L260P of SEQ ID NO:284). In illustrative embodiments of lymphoproliferative elements that include a first intracellular domain derived from MyD88, the second intracellular domain can be derived from TNFRSF4 or TNFRSF8. In other illustrative embodiments of lymphoproliferative elements that include a first intracellular domain derived from MyD88, the second intracellular domain can be other than an intracellular domain derived from a CD28 family member (e.g., CD28, ICOS), Pattern Recognition Receptor, a C-reactive protein receptor, a TNF receptor, or an HSP receptor.

In some embodiments, a cell expressing the lymphoproliferative element comprising an intracellular and transmembrane domain of MPL can be contacted with or exposed to eltrombopag, or a patient or subject to which such a cell has been infused can be treated with eltrombopag. Not to be limited by theory, eltrombopag binds to the transmembrane domain of MPL and induces the activation of the intracellular domain of MPL.

The domains, motifs, and point mutations of MPL that induce proliferation and/or survival of T cells and/or NK cells are known in the art and a skilled artisan can identify corresponding domains, motifs, and point mutations in MPL polypeptides, some of which are discussed in this paragraph. Deletion of the region encompassing amino acids 70-95 in SEQ ID NO:283 was shown to support viral transformation in the context of v-mpl (Benit et al. J Virol. 1994 August; 68(8):5270-4), thus indicating that this region is not necessary for the function of mpl in this context. Morello et al. Blood 1995 July; 86(8):557-71 used the same deletion to show that this region was not required for stimulating transcription for a hematopoietin receptor-responsive CAT reporter gene construct and furthermore saw that this deletion resulted in slightly enhanced transcription expected for removal of a nonessential and negative element in this region as suggested by Drachman and Kaushansky. Thus, in some embodiments, a MPL intracellular signaling domain does not comprise the region comprising amino acids 70-95 in SEQ ID NO:283. Using computer simulations, Lee et al. found clinically relevant mutations in the transmembrane domain of MPL should activate MPL with the following order of activating effects: W515K (corresponding to the amino acid substitution W2K of SEQ ID NO: 283)>S505A (corresponding to the amino acid substitution S14A of SEQ ID NO:187)>W5151 (corresponding to the amino acid substitution W21 of SEQ ID NO: 283)>S505N (corresponding to the amino acid substitution S14N of SEQ ID NO:187, which was tested as part T075 (SEQ ID NO:188)) (Lee et. a. PLoS One. 2011; 6(8): e23396). The simulations predicted these mutations could cause constitutive activation of JAK2, the kinase partner of MPL. In some embodiments, the intracellular portion of MPL can include one or more, or all the domains and motifs described herein that are present in SEQ ID NO: 283. In some embodiments, a transmembrane portion of MPL can include one or more, or all the domains and motifs described herein that are present in SEQ ID NO:187. In illustrative embodiments of lymphoproliferative elements that include a first intracellular domain derived from MPL, the second intracellular domain can be derived from CD79B.

In illustrative embodiments of lymphoproliferative elements that include a second intracellular domain derived from CD79B, the first intracellular domain can be derived from CSF3R.

In some embodiments, a lymphoproliferative element that includes an PRLR intracellular domain can include the growth hormone receptor binding domain of PRLR and any known mutations (growth hormone receptor binding domain at residues 28-104 of SEQ ID NO:295).

In some embodiments, a lymphoproliferative element that includes an ICOS intracellular domain can include a calcium-signaling motif (calcium-signaling motif at residues 5-8 of SEQ ID NO:225). In some embodiments, a lymphoproliferative element that includes an ICOS intracellular domain can include at least one of a first and second conserved motif (first and second conserved motifs at residues 9-18 and 24-30, respectively, of SEQ ID NO:225). In some embodiments, a lymphoproliferative element that includes an ICOS intracellular domain does not include at least one of the first and second conserved motif.

EPOR also contains a short segment important for EPOR internalization (residues 267-276 of full-length EPOR). In some embodiments, a lymphoproliferative element that includes an EPOR intracellular domain does not include the internalization segment.

The domains, motifs, and point mutations of intracellular signaling domains that induce proliferation and/or survival of T cells and/or NK cells are known in the art and a skilled artisan can identify corresponding domains, motifs, and point mutations in polypeptides, some of which are above, and a skilled artisan can identify corresponding domains, motifs, and point mutations in other polypeptides. A skilled artisan will be able to identify these domains, motifs, and point mutations in similar polypeptides using, for example, sequence alignments to known binding motifs. In some embodiments, a lymphoproliferative element herein can include any, for example, one or more up to all of the domains, motifs, and mutations of a intracellular signaling domain disclosed herein or otherwise known to induce proliferation and/or survival of T cells and/or NK cells.

In another embodiment, the LE provides, is capable of providing and/or possesses the property of (or a cell modified, genetically modified, and/or transduced with the LE is capable of providing, is adapted for, possesses the property of, and/or is modified for) driving T cell expansion in vivo.

In some embodiments, the lymphoproliferative element can include any of the sequences listed in Table 1 (SEQ ID NOs: 84-302). Table 1 shows the parts, names (including gene names), and amino acid sequences for domains that were tested in CLEs. CLEs can include in certain illustrative embodiments, an extracellular domain (denoted P1), a transmembrane domain (denoted P2), a first intracellular domain (denoted P3), and a second intracellular domain (denoted P4). Typically, the lymphoproliferative element includes a first intracellular domain In illustrative embodiments, the first intracellular domain can include any of the parts listed as S036 to S0216 or in Table 1, or functional mutants and/or fragments thereof. In some embodiments, the lymphoproliferative element can include a second intracellular domain In illustrative embodiments, the second intracellular domain can include any of the parts listed as S036 to S0216 or in Table 1, or functional mutants and/or fragments thereof. In some embodiments, the lymphoproliferative element can include an extracellular domain In illustrative embodiments, the extracellular domain can include any of the sequences of parts listed as M001 to M049 or E006 to E015 in Table 1, or functional mutants and/or fragments thereof. In some embodiments, the lymphoproliferative element can include a transmembrane domain. In illustrative embodiments, the transmembrane domain can include any of the parts listed as M001 to M049 or T001 to T082 in Table 1, or functional mutants and/or fragments thereof. In some embodiments, the lymphoproliferative element can be of fusion of an extracellular/transmembrane domain (M001 to M049 in Table 1), a first intracellular domain (S036 to S0216 in Table 1), and a second intracellular domain (S036 to S216 in Table 1). In some embodiments, the lymphoproliferative element can be a fusion of an extracellular domain (E006 to E016 in Table 1), a transmembrane domain (T001 to T082 in Table 1), a first intracellular domain (S036 to S0216 in Table 1), and a second intracellular domain (S036 to S0216 in Table 1). For example, the lymphoproliferative element can be a fusion of E006, T001, S036, and S216, also written as E006-T001-S036-S216). In illustrative embodiments, the lymphoproliferative element can be the fusion E010-T072-S192-S212, E007-T054-S197-S212, E006-T006-S194-S211, E009-T073-S062-S053, E008-T001-S121-S212, E006-T044-S186-S053, or E006-T016-S186-S050.

In illustrative embodiments, the intracellular domain of an LE, or the first intracellular domain in an LE that has two or more intracellular domains, is other than a functional intracellular activating domain from an ITAM-containing intracellular domain, for example, an intracellular domain from CD3Z, CD3D, CD3E, CD3G, CD79A, CD79B, DAP12, FCER1G, FCGR2A, FCGR2C, DAP10/CD28, or ZAP70, and in a further illustrative subembodiment, CD3z. In illustrative embodiments, the extracellular domain of an LE does not comprise a single-chain variable fragment (scFv). In further illustrative embodiments, the extracellular domain of an LE that upon binding to a binding partner activates an LE, does not comprise a single-chain variable fragment (scFv). A CLE does not comprise both an ASTR and an activation domain from CD3Z, CD3D, CD3E, CD3G, CD79A, CD79B, DAP12, FCER1G, FCGR2A, FCGR2C, DAP10/CD28, or ZAP70. If an LE does include an ASTR (and not an activation domain in the previous list), the ASTR of an LE in illustrative embodiments does not include an scFv. In some embodiments, a lymphoproliferative element does not include an extracellular domain

In some embodiments, the lymphoproliferative element, and in illustrative embodiments CLE, is not covalently attached to a cytokine. In some aspects, a lymphoproliferative element, and in illustrative embodiments CLE, comprises a cytokine polypeptide covalently linked to its cognate receptor. In either of these embodiments, the CLE can be constitutively active and typically constitutively activates the same Jak/STAT and/or TRAF pathways as the corresponding activated wild-type cytokine receptor. In some embodiments, the chimeric cytokine receptor is an interleukin. In some embodiments, the CLE is IL-7 covalently linked to IL7RA or IL-15 covalently linked to IL15RA. In other embodiments, the CLE is other than IL-15 covalently linked to IL15RA. In other aspects, the CLE comprises a cytokine polypeptide covalently linked to only a portion of its cognate receptor that includes a functional portion of the extracellular domain capable of binding the cytokine polypeptide, the transmembrane domain and/or intracellular domain are from heterologous polypeptides, and the CLE is constitutively active. In one embodiment, the CLE is IL-7 covalently linked to the extracellular and transmembrane domains of IL7RA, and the intracellular domain from IL2RB. In another embodiment, the CLE is a cytokine polypeptide covalently linked to a portion of its cognate receptor that includes a functional portion of the extracellular domain capable of binding the cytokine polypeptide, a heterologous transmembrane domain, and lymphoproliferative element intracellular domain provided herein. In some embodiments, the lymphoproliferative element is a cytokine receptor that is not tethered to a cytokine.

In some aspects, the lymphoproliferative element is capable of binding to soluble cytokines or growth factors and such binding is required for activity. In certain illustrative embodiments, the lymphoproliferative element is constitutively active, and thus does not require binding to a soluble growth factor or cytokine for activity. Typically, constitutively active lymphoproliferative elements do not bind soluble cytokines or growth factors. In some embodiments, the lymphoproliferative element is a chimera comprising an extracellular binding domain from one receptor and the intracellular signaling domain from a different receptor. In some embodiments the CLE is an inverted receptor that is activated upon binding of a ligand that would inhibit proliferation and/or survival when bound to its natural receptor, but instead leads to proliferation and/or survival upon activating the CLE. In some embodiments, inverted receptors include chimeras that comprise an extracellular ligand binding domain from IL4Ra and an intracellular domain from IL7Ra or IL21. Other embodiments of inverted cytokine receptors include chimeras that comprise an extracellular ligand binding domain from a receptor that would inhibit proliferation and/or survival when bound to its natural ligand, such as receptors for IL-4, IL-10, IL-13, or TGFb, and any lymphoproliferative element intracellular domain disclosed herein. In illustrative aspects, the lymphoproliferative element does not bind a cytokine. In further illustrative aspects, the lymphoproliferative element does not bind any ligand. In illustrative embodiments, the lymphoproliferative elements that do not bind any ligand are constitutively dimerized or otherwise multimerized, and are constitutively active. In illustrative embodiments of any of the methods and compositions provided herein that include a lymphoproliferative element, the intracellular domain can be derived from an intracellular portion of the transmembrane protein of the TNF receptor family, CD40. The domains, motifs, and point mutations of CD40 that induce proliferation and/or survival of T cells and/or NK cells are known in the art and a skilled artisan can identify corresponding domains, motifs, and point mutations in CD40 polypeptides, some of which are discussed in this paragraph. The CD40 protein contains several binding sites for TRAF proteins. Not to be limited by theory, binding sites for TRAF1, TRAF2, and TRAF3 are located at the membrane distal domain of the intracellular portion of CD40 and include the amino acid sequence PXQXT (SEQ ID NO:303) where each X can be any amino acid, (corresponding to amino acids 35-39 of SEQ ID NO:208) (Elgueta et al. Immunol Rev. 2009 May; 229(1):152-72). TRAF2 has also been shown to bind to the consensus sequence SXXE (SEQ ID NO:304) where each X can be any amino acid, (corresponding to amino acids 57-60 of SEQ ID NO:208) (Elgueta et al. Immunol Rev. 2009 May; 229(1):152-72). A distinct binding site for TRAF6 is situated at the membrane proximal domain of intracellular portion of CD40 and includes the consensus sequence QXPXEX (SEQ ID NO:305) where each X can be any amino acid (corresponding to amino acids 16-21 of SEQ ID NO:208) (Lu et al. J Biol Chem. 2003 Nov. 14; 278(46):45414-8). In illustrative embodiments, the intracellular portion of the transmembrane protein CD40 can include all the binding sites for the TRAF proteins. The TRAF binding sites are known in the art and a skilled artisan will be able to identify corresponding TRAF binding sites in similar CD40 polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:208 or SEQ ID NO:209. In some embodiments, the intracellular domain derived from CD40 has a length of from about 30 amino acids (aa) to about 35 aa, from about 35 aa to about 40 aa, from about 40 aa to about 45 aa, from about 45 aa to about 50 aa, from about 50 aa to about 55 aa, from about 55 aa to about 60 aa, or from about 60 aa to about 65 aa. In illustrative embodiments, the intracellular domain derived from CD40 has a length of from about 30 aa to about 66 aa, for example, 30 aa to 65 aa, or 50 aa to 66 aa. In illustrative embodiments of lymphoproliferative elements that include a first intracellular domain derived from CD40, the second intracellular domain can be other than an intracellular domain derived from MyD88, a CD28 family member (e.g., CD28, ICOS), Pattern Recognition Receptor, a C-reactive protein receptor (i.e., Nodi, Nod2, PtX3-R), a TNF receptor, CD40, RANK/TRANCE-R, OX40, 4-1BB), an HSP receptor (Lox-1 and CD91), or CD28. Pattern Recognition Receptors include, but are not limited to endocytic pattern-recognition receptors (i.e., mannose receptors, scavenger receptors (i.e., Mac-1, LRP, peptidoglycan, teichoic acids, toxins, CD1 1 c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), and RIG1.

In illustrative embodiments of any of the methods and compositions provided herein that include a lymphoproliferative element, the intracellular domain can be derived from a portion of the transmembrane protein MPL. Accordingly, in some embodiments, the lymphoproliferative element comprises MPL, or is MPL, or a variant and/or fragment thereof, including a variant and/or fragment that includes at least 75, 80, 85, 90, 95, 96, 97, 98, 99, or 100% of the intracellular domain of MPL, with or without a transmembrane and/or extracellular domain of MPL, wherein the variant and/or fragment retains the ability to promote cell proliferation of PBMCs, and in some embodiments T cells. In some embodiments, a cell expressing the lymphoproliferative element comprising an intracellular and transmembrane domain of MPL can be contacted with, exposed to, or treated with eltrombopag. Not to be limited by theory, eltrombopag binds to the transmembrane domain of MPL and induces the activation of the intracellular domain of MPL. The domains, motifs, and point mutations of MPL that induce proliferation and/or survival of T cells and/or NK cells are known in the art and a skilled artisan can identify corresponding domains, motifs, and point mutations in MPL polypeptides, some of which are discussed in this paragraph. The transmembrane MPL protein contains the Box1 motif PXXP (SEQ ID NO:306) where each X can be any amino acid (corresponding to amino acids 17-20 in SEQ ID NO:283) and the Box2 motif, a region with increased serine and glutamic acid content (corresponding to amino acids 46-64 in SEQ ID NO:283) (Drachman and Kaushansky. Proc Natl Acad Sci USA. 1997 Mar. 18; 94(6):2350-5). The Box1 and Box2 motifs are involved in binding to JAKs and signal transduction, although the Box2 motif presence is not always required for a proliferative signal (Murakami et al. Proc Natl Acad Sci USA. 1991 Dec. 15; 88(24):11349-53; Fukunaga et al. EMBO J. 1991 October; 10(10):2855-65; and O′Neal and Lee. Lymphokine Cytokine Res. 1993 October; 12(5):309-12). Many cytokine receptors have hydrophobic residues at positions −1, −2, and −6 relative to the Box1 motif (corresponding to amino acids 16, 15, and 11, respectively, of SEQ ID NO:283), that form a “switch motif,” which is required for cytokine-induced JAK2 activation but not for JAK2 binding (Constantinescu et al. Mol Cell. 2001 February; 7(2):377-85; and Huang et al. Mol Cell. 2001 December; 8(6):1327-38). Deletion of the region encompassing amino acids 70-95 in SEQ ID NO:283 was shown to support viral transformation in the context of v-mpl (Benit et al. J Virol. 1994 August; 68(8):5270-4), thus indicating that this region is not necessary for the function of mpl in this context. Morello et al. Blood 1995 July; 86(8):557-71 used the same deletion to show that this region was not required for stimulating transcription for a hematopoietin receptor-responsive CAT reporter gene construct and furthermore saw that this deletion resulted in slightly enhanced transcription expected for removal of a nonessential and negative element in this region as suggested by Drachman and Kaushansky. Thus, in some embodiments, a MPL intracellular signaling domain does not comprise the region comprising amino acids 70-95 in SEQ ID NO:283. In full-length MPL, the lysines K553 (corresponding to K40 of SEQ ID NO: 283) and K573 (corresponding to K60 of SEQ ID NO: 283) have been shown to be negative regulatory sites that function as part of a ubiquitination targeting motif (Saur et al. Blood 2010 Feb. 11;115(6):1254-63). Thus, in some embodiments herein, a MPL intracellular signaling domain does not comprise these ubiquitination targeting motif residues. In full-length MPL, the tyrosines Y521 (corresponding to Y8 of SEQ ID NO: 283), Y542 (corresponding to Y29 of SEQ ID NO:283), Y591 (corresponding to Y78 of SEQ ID NO: 283), Y626 (corresponding to Y113 of SEQ ID NO: 283), and Y631 (corresponding to Y118 of SEQ ID NO: 283) have been shown to be phosphorylated (Varghese et al. Front Endocrinol (Lausanne). 2017 Mar. 31; 8:59). Y521 and Y591 of full-length MPL are negative regulatory sites that function either as part of a lysosomal targeting motif (Y521) or via an interaction with adaptor protein AP2 (Y591) (Drachman and Kaushansky. Proc Natl Acad Sci USA. 1997 Mar. 18; 94(6):2350-5; and Hitchcock et al. Blood. 2008 Sep. 15; 112(6):2222-31). Y626 and Y631 of full-length MPL are positive regulatory sites (Drachman and Kaushansky. Proc Natl Acad Sci USA. 1997 Mar. 18; 94(6):2350-5) and the murine homolog of Y626 is required for cellular differentiation and the phosphorylation of Shc (Alexander et al. EMBO J. 1996 Dec. 2; 15(23):6531-40) and Y626 is also required for constitutive signaling in MPL with the W515A mutation described below (Pecquet et al. Blood. 2010 Feb. 4; 115(5):1037-48). MPL contains the Shc phosphotyrosine-binding binding motif NXXY (SEQ ID NO:307) where each X can be any amino acid (corresponding to amino acids 110-113 of SEQ ID NO: 283), and this tyrosine is phosphorylated and important for the TPO-dependent phosphorylation of Shc, SHIP, and STAT3 (Laminet et al. J Biol Chem. 1996 Jan. 5; 271(1):264-9; and van der Geer et al. Proc Natl Acad Sci USA. 1996 Feb. 6; 93(3):963-8). MPL also contains the STAT3 consensus binding sequence YXXQ (SEQ ID NO:308) where each X can be any amino acid (corresponding to amino acids 118-121 of SEQ ID NO: 283) (Stahl et al. Science. 1995 Mar. 3; 267(5202):1349-53). The tyrosine of this sequence can be phosphorylated and MPL is capable of partial STAT3 recruitment (Drachman and Kaushansky. Proc Natl Acad Sci USA. 1997 Mar. 18; 94(6):2350-5). MPL also contains the sequence YLPL (SEQ ID NO: 309) (corresponding to amino acid 113-116 of SEQ ID NO: 283), which is similar to the consensus binding site for STATS recruitment pYLXL (SEQ ID NO:310) where pY is phosphotyrosine and X can be any amino acid (May et al. FEBS Lett. 1996 Sep. 30; 394(2):221-6). Using computer simulations, Lee et al. found clinically relevant mutations in the transmembrane domain of MPL should activate MPL with the following order of activating effects: W515K (corresponding to the amino acid substitution W2K of SEQ ID NO: 283)>S505A (corresponding to the amino acid substitution S14A of SEQ ID NO:187)>W515I (corresponding to the amino acid substitution W2I of SEQ ID NO: 283)>S505N (corresponding to the amino acid substitution S14N of SEQ ID NO:187, which was tested as part T075 (SEQ ID NO:188)) (Lee et. a. PLoS One. 2011; 6(8): e23396). The simulations predicted these mutations could cause constitutive activation of JAK2, the kinase partner of MPL. In some embodiments, the intracellular portion of MPL can include one or more, or all the domains and motifs described herein that are present in SEQ ID NO 283. In some embodiments, a transmembrane portion of MPL can include one or more, or all the domains and motifs described herein that are present in SEQ ID NO:187. The domains, motifs, and point mutations of MPL provided herein are known in the art and a skilled artisan would recognize that MPL intracellular signaling domains herein in illustrative embodiments would include one or more corresponding domains, motifs, and point mutations in that have been shown to promote proliferative activity and would not include that that have been shown to inhibit MPLs proliferative activity. Any or all of these domains, motifs, and point mutations of MPL can be present in an intracellular signaling domain can be included in any of the aspects and embodiments disclosed herein. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:283. In some embodiments, the intracellular domain derived from MPL has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, from about 250 aa to 300 aa, from about 300 aa to 350 aa, from about 350 aa to about 400 aa, from about 400 aa to about 450 aa, from about 450 aa to about 500 aa, from about 500 aa to about 550 aa, from about 550 aa to about 600 aa, or from about 600 aa to about 635 aa. In illustrative embodiments, the intracellular domain derived from MPL has a length of from about 30 aa to about 200 aa, for example, 30 aa to 150 aa, 30 aa to 119 aa, 30 aa to 121 aa, 30 aa to 122 aa, or 50 aa to 125 aa. In illustrative embodiments of lymphoproliferative elements that include a first intracellular domain derived from MPL, the second intracellular domain can be derived from CD79B.

Lymphoproliferative elements and CLEs that can be included in any of the aspects disclosed herein, can be any of the LEs or CLEs disclosed in WO2019/055946. CLEs were disclosed therein that promoted proliferation in cell culture of PBMCs that were transduced with lentiviral particles encoding the CLEs between day 7 and day 21, 28, 35 and/or 42 after transduction. Furthermore, CLEs were identified therein, that promoted proliferation in vivo in mice in the presence or absence of an antigen recognized by a CAR, wherein T cells expressing one of the CLEs and the CAR were introduced into the mice. As exemplified therein, tests and/or criteria can be used to identify whether any test polypeptide, including LEs, or test domains of an LE, such as a first intracellular domain, or a second intracellular domain, or both a first and second intracellular domain, are indeed LEs or effective intracellular domains of LEs, or especially effective LEs or intracellular domains of LEs. Thus, in certain embodiments, any aspect or other embodiment provided herein that includes an LE or a polynucleotide or nucleic acid encoding an LE can recite that the LE meets, or provides the property of, or is capable of providing and/or possesses the property of, any one or more of the identified tests or criteria for identifying an LE provided herein, or that a cell genetically modified, transduced, and/or stably transfected with a recombinant nucleic acid vector, such as a cell that is transduced with a lentiviral particle encoding the LE, is capable of providing, is adapted for, possesses the property of, and/or is modified for achieving the results of one or more of the recited tests. In one embodiment, the LE provides, is capable of providing and/or possesses the property of, (or a cell genetically modified and/or transduced with a retroviral particle encoding the LE is capable of providing, is adapted for, possesses the property of, and/or is modified for) improved expansion to pre-activated PBMCs transduced with a lentivirus comprising a nucleic acid encoding the LE and an anti-CD19 CAR comprising a CD3 zeta intracellular activating domain but no co-stimulatory domain, between day 7 and day 21, 28, 35, and/or 42 of in vitro culturing post-transduction in the absence of exogenously added cytokines, compared to a control retroviral particle, e.g. lentiviral particle under identical conditions. In some embodiments, a lymphoproliferative element test for improved or enhanced survival, expansion, and/or proliferation of cells transduced with a retroviral particle (e.g. lentiviral particle) having a genome encoding a test construct encoding a putative LE (test cells) can be performed based on a comparison to control cells, which can be, for example, either untransduced cells or cells transduced with a control retroviral (e.g. lentiviral) particle identical to the lentiviral particle comprising the nucleic acid encoding the lymphoproliferative element, but lacking the lymphoproliferative element, or lacking the intracellular domain or domains of the test polypeptide construct but comprising the same extracellular domain, if present, and the same transmembrane region or membrane targeting region of the respective test polypeptide construct. In some embodiments control cells are transduced with a retroviral particle (e.g., lentiviral particle) having a genome encoding a lymphoproliferative element or intracellular domain(s) thereof, identified herein as exemplifying a lymphoproliferative element. In such an embodiment, the test criteria can include that there is at least as much enrichment, survival and/or expansion, or no statistical difference of enrichment, survival, and/or expansion when the test is performed using a retroviral particle (e.g., lentiviral particle) having a genome encoding a test construct versus encoding the control lymphoproliferative element, typically by analyzing cells transcribed therewith. Exemplary or illustrative embodiments of lymphoproliferative elements herein, in some embodiments, are illustrative embodiments of control lymphoproliferative elements for such a test.

In some embodiments, this test for an improved property of a putative or test lymphoproliferative element is performed by performing replicates and/or performing a statistical test. A skilled artisan will recognize that many statistical tests can be used for such a lymphoproliferative element test. Contemplated for such a test in these embodiments would be any such test known in the art. In some embodiments, the statistical test can be a T-test or a Mann-Whitney-Wilcoxon test. In some embodiments, the normalized enrichment level of a test construct is significant at a p-value of less than 0.1, or less than 0.05, or less than 0.01.

In another embodiment, the LE provides, is capable of providing and/or possesses the property of (or a cell genetically modified and/or transduced with the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold expansion, or between 1.5 fold and 25-fold expansion, or between 2-fold and 20-fold expansion, or between 2-fold and 15-fold expansion, or between 5-fold and 25-fold expansion, or between 5-fold and 20-fold expansion, or between 5-fold and 15-fold expansion, of pre-activated PBMCs transduced with a nucleic acid encoding the LE when transduced along with an anti-CD19 CAR comprising a CD3 zeta intracellular activating domain but no co-stimulatory domain, between day 7 and day 21, 28, 35, and/or 42 of in vitro culturing in the absence of exogenously added cytokines. In some embodiments, the test is performed in the presence of PBMCs, for example at a 1:1 ratio of transduced cells to PBMCs, which can be for example, from a matched donor, and in some embodiments, the test is performed in the absence of PBMCs. In some embodiments, the analysis of expansion for any of these tests is performed as illustrated in WO2019/055946. In some embodiments, the test can include a further statistical test and a cut-off such as a P value below 0.1, 0.05, or 0.01, wherein a test polypeptide or nucleic acid encoding the same, needs to meet one or both thresholds (i.e., fold expansion and statistical cutoff).

For any of the lymphoproliferative element tests provided herein, the number of test cells and the number of control cells can be compared between day 7 and day 14, 21, 28, 35, 42 or 60 post-transduction. In some embodiments, the numbers of test and control cells can be determined by sequencing DNA and counting the occurrences of unique identifiers present in each construct. In some embodiments, the numbers of test and control cells can be counted directly, for example with a hemocytometer or a cell counter. In some embodiments, all the test cells and control cells can be grown within the same vessel, well or flask. In some embodiments, the test cells can be seeded in one or more wells, flasks or vessels, and the control cells can be seeded in one or more flasks or vessels. In some embodiments, test and control cells can be seeded individually into wells or flasks, e.g., one cell per well. In some embodiments, the numbers of test cells and control cells can be compared using enrichment levels. In some embodiments, the enrichment level for a test or control construct can be calculated by dividing the number of cells at a later time point (day 14, 21, 28, 35, or day 45) by the number of cells at day 7 for each construct. In some embodiments, the enrichment level for a test or control construct can be calculated by dividing the number of cells at a time point (day 14, 21, 28, 35, or day 45) by the number of cells at that time point for untransduced cells. In some embodiments, the enrichment level of each test construct can be normalized to the enrichment level of the respective control construct to generate a normalized enrichment level. In some embodiments, a LE encoded in the test construct provides (or a cell genetically modified and/or transduced with a retroviral particle (e.g. lentiviral particle) having a genome encoding the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold normalized enrichment level, or between 1.5 fold and 25-fold normalized enrichment level, or between 3-fold and 20-fold normalized enrichment level, or between 5-fold and 25-fold normalized enrichment level, or between 5-fold and 20-fold normalized enrichment level, or between 5-fold and 15-fold normalized enrichment level. Enrichment can be measured, for example, by direct cell counting. Cutoff values can be based on a single test, or two, three, four, or five repeats, or based on many repeats. The cutoff can be met when a lymphoproliferative element meets one or more repeat tests, or meets or exceeds a cutoff for all repeats. In some embodiments, the enrichment is measured as log₂((normalized count data on the test day+1)/(normalized count data on day 7+1)).

Additional details regarding the tests performed to identify the LEs are illustrated in WO2019/055946, including experimental conditions.

As illustrated in WO2019/055946, test constructs were identified as CLEs because the CLEs induced proliferation/expansion in these fed or unfed cultures without added cytokines such as IL-2 between days 7 and day 21, 28, 35, and/or 42. For example, as illustrated in WO2019/055946, effective CLEs were identified by identifying test CLEs that provided increased expansion of these in vitro cultures, whether fed or unfed with untransduced PBMCs, between day 7 and day 21, 28, 35, and/or 42 post-transduction, compared to control constructs that did not include any intracellular domains. WO2019/055946 discloses that at least one and typically more than one test CLE that included an intracellular domain from a test gene provided more expansion than every control construct that was present at day 7 post-transduction, that did not include an intracellular domain WO2019/055946 further provides a statistical method that was used to identify exceptionally effective genes with respect to a first intracellular domain, and one or more exemplary intracellular domain(s) from these genes. The method used a Mann-Whitney-Wilcoxon test and a false discovery cutoff rate of less than 0.1 or less than 0.05. WO2019/055946 identified especially effective genes for the first intracellular domain or the second intracellular domain, for example, by analyzing scores for genes calculated as combined score for all constructs with that gene. Such analysis can use a cutoff of greater than 1, or greater than negative control constructs without any intracellular domains, or greater than 2, as shown for some of the tests disclosed in WO2019/055946.

In another embodiment, the LE provides, is capable of providing and/or possesses the property of (or a cell genetically modified and/or transduced with the LE is capable of providing, is adapted for, possesses the property of, and/or is modified for) driving T cell expansion in vivo. For example, the in vivo test can utilize a mouse model and measure T cell expansion at 15 to 25 days in vivo, or at 19 to 21 days in vivo, or at approximately 21 days in vivo, after T cells are contacted with lentiviral vectors encoding the LEs, are introduced into the mice, as disclosed in WO2019/055946,

In exemplary aspects and embodiments that include a LE, which typically include a CAR, such as methods provided herein for modifying, genetically modifying and/or transducing cells, and uses thereof, the genetically modified cell is modified so as to possess new properties not previously possessed by the cell before genetic modification and/or transduction. Such a property can be provided by genetic modification with a nucleic acid encoding a CAR or a LE, and in illustrative embodiments both a CAR and a LE. For example, in certain embodiments, the genetically modified and/or transduced cell is capable of, is adapted for, possesses the property of, and/or is modified for survival and/or proliferation in ex vivo culture for at least 7, 14, 21, 28, 35, 42, or 60 days or from between day 7 and day 14, 21, 28, 35, 42 or 60 post-transduction, in the absence of added IL-2 or in the absence of added cytokines such as IL-2, IL-15, or IL-7, and in certain illustrative embodiments, in the presence of the antigen recognized by the CAR where the method comprises modifying using a retroviral particle having a pseudotyping element and optionally a separate or fused activation domain on its surface and typically does not require pre-activation.

By capable of enhanced survival and/or proliferation in certain embodiments, it is meant that the genetically modified and/or transduced cell exhibits, is capable of, is adapted for, possesses the property of, and/or is modified for improved survival or expansion in ex vivo or in vitro culture in culture media in the absence of one or more added cytokines such as IL-2, IL-15, or IL-7, or added lymphocyte mitogenic agent, compared to a control cell(s) identical to the genetically modified and/or transduced cell(s) before it was genetically modified and/or transduced or to a control cell that was transduced with a retroviral particle identical to an on-test retroviral particle that comprises an LE or a putative LE, but without the LE or the intracellular domains of the LE, wherein said survival or proliferation of said control cell(s) is promoted by adding said one or more cytokines, such as IL-2, IL-15, or IL-7, or said lymphocyte mitogenic agent to the culture media. By added cytokine or lymphocyte mitogenic agent, it is meant that cytokine or lymphocyte mitogenic agent is added from an exogenous source to a culture media such that the concentration of said cytokine or lymphocyte mitogenic agent is increased in the culture media during culturing of the cell(s) compared to the initial culture media, and in some embodiments can be absent from the initial culture media before said adding. By “added” or “exogenously added”, it is meant that such cytokine or lymphocyte mitogenic agent is added to a lymphocyte media used to culture the modified, genetically modified, and/or transduced cell after the modifying, where the culture media may or may not already possess the cytokine or lymphocyte mitogenic agent. All or a portion of the media that includes a mixture of multiple media components is typically stored and in illustrative embodiments has been shipped to a site where the culturing takes place, without the exogenously added cytokine(s) or lymphocyte mitogenic agent(s). The lymphocyte media in some embodiments is purchased from a supplier, and a user such as a technician not employed by the supplier and not located within a supplier facility, adds the exogenously added cytokine or lymphocyte mitogenic agent to the lymphocyte media and then the genetically modified and/or transduced cells are cultured in the presence or absence of such exogenously added cytokine or lymphocyte mitogenic agent.

In some embodiments, improved or enhanced survival, expansion, and/or proliferation can be shown as an increase in the number of cells determined by sequencing DNA from cells transduced with retroviral particle (e.g. lentiviral particle) having a genome encoding CLEs and counting the occurrences of sequences present in unique identifiers from each CLE. In some embodiments, improved survival and/or improved expansion can be determined by counting the cells directly, for example with a hemocytometer or a cell counter, at each time point. In some embodiments, improved survival and/or improved expansion and/or enrichment can be calculated by dividing the number of cells at the later time point (day 21, 28, 35, and/or day 45) by the number of cells at day 7 for each construct. In some embodiments, the cells can be counted by hemocytometer or cell counters. In some embodiments, the enrichment level determined using the nucleic acid counts or the cell counts of each specific test construct can be normalized to the enrichment level of the respective control construct, i.e., the construct with the same extracellular domain and transmembrane domain but lacking the intracellular domains present in the test construct. In these embodiments, the LE encoded in the construct provides (or a cell genetically modified and/or transduced with a retroviral particle (e.g. lentiviral particle) having a genome encoding the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold normalized enrichment level, or between 1.5 fold and 25-fold normalized enrichment level, or between 3-fold and 20-fold normalized enrichment level, or between 5-fold and 25-fold normalized enrichment level, or between 5-fold and 20-fold normalized enrichment level, or between 5-fold and 15-fold normalized enrichment level.

In some embodiments, the lymphoproliferative element can include a cytokine receptor or a fragment that includes a signaling domain thereof. In some embodiments, the cytokine receptor can be CD27, CD40, CRLF2, CSF2RA, CSF2RB, CSF3R, EPOR, GHR, IFNAR1, IFNAR2, IFNGR1, IFNGR2, IFNLR1, IL1R1, IL1RAP, IL1RL1, IL1RL2, IL2R, IL2RA, IL2RB, IL2RG, IL3RA, IL4R, IL5RA, IL6R, IL6ST, IL7R, IL7RA, IL9R, IL10RA, IL10RB, IL11RA, IL12RB1, IL13R, IL13RA1, IL13RA2, IL15R, IL15RA, IL17RA, IL17RB, IL17RC, IL17RE, IL18R1, IL18RAP, IL20RA, IL20RB, IL21R, IL22RA1, IL23R, IL27R, IL27RA, IL31RA, LEPR, LIFR, MPL, OSMR, PRLR, TGFβR, TGFβ decoy receptor, TNFRSF4, TNFRSF8, TNFRSF9, TNFRSF14, or TNFRSF18.

In some embodiments, a lymphoproliferative element, including a CLE, comprises an intracellular activating domain as disclosed hereinabove. In some illustrative embodiments a lymphoproliferative element is a CLE comprising an intracellular activating domain comprising an ITAM-containing domain, as such, the CLE can comprise an intracellular activating domain having at least 80%, 90%, 95%, 98%, or 100% sequence identity to the CD3Z, CD3D, CD3E, CD3G, CD79A, CD79B, DAP12, FCER1G, FCGR2A, FCGR2C, DAP10/CD28, or ZAP70 domains provided herein wherein the CLE does not comprise an ASTR.

In some embodiments, one or more domains of a lymphoproliferative element is fused to a modulatory domain, such as a co-stimulatory domain, and/or an intracellular activating domain of a CAR. In some embodiments of the composition and method aspects for transducing lymphocytes in whole blood, one or more intracellular domains of a lymphoproliferative element can be part of the same polypeptide as a CAR or can be fused and optionally functionally connected to some components of CARs. In still other embodiments, an engineered signaling polypeptide can include an ASTR, an intracellular activation domain (such as a CD3 zeta signaling domain), a co-stimulatory domain, and a lymphoproliferative domain Further details regarding co-stimulatory domains, intracellular activating domains, ASTRs and other CAR domains, are disclosed elsewhere herein.

Lymphoproliferative elements provided herein typically include a transmembrane domain For example, the transmembrane domain can have 80%, 85%, 90%, 95%, 97%, 98%, 99% or 100% sequence identity to any one of the transmembrane domains from the following genes and representative sequences disclosed in WO2019/055946: CD8 beta, CD4, CD3 zeta, CD28, CD134, CD7, CD2, CD3D, CD3E, CD3G, CD3Z, CD4, CD8A CD8B, CD27, CD28, CD40, CD79A, CD79B, CRLF2, CRLF2, CSF2RA, CSF2RB, CSF2RB, CSF3R, EPOR, FCER1G, FCGR2C, FCGRA2, GHR, ICOS, IFNAR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL27RA, IL31RA, LEPR, LIFR, MPL, OSMR, PRLR, TNFRSF4, TNFRSF8, TNFRSF9, TNFRSF14, and TNFRSF18 or mutants thereof that are known to promote signaling activity in certain cell types if such mutants. Transmembrane (TM) domains suitable for use in any engineered signaling polypeptide include, but are not limited to, constitutively active cytokine receptors, the TM domain from LMP1, and TM domains from type 1 TM proteins comprising a dimerizing motif, as discussed in more detail herein. In any of the aspects disclosed herein containing the transmembrane domain from a type I transmembrane protein, the transmembrane domain can be a Type I growth factor receptor, a hormone receptor, a T cell receptor, or a TNF-family receptor.

In some embodiments, CLEs include both an extracellular portion and a transmembrane portion that is from the same protein, in illustrative embodiments the same receptor, either of which in illustrative embodiments is a mutant, thus forming an extracellular and transmembrane domain These domains can be from a cytokine receptor, or a mutant thereof, or a hormone receptor, or a mutant thereof in some embodiments that have been reported to be constitutively active when expressed at least in some cell types. In illustrative embodiments, such extracellular and transmembrane domains do not include a ligand binding region. It is believed that such domains do not bind a ligand when present in CLEs and expressed in B cells, T cells, and/or NK cells. Mutations in such receptor mutants can occur in the transmembrane region or in the extracellular juxtamembrane region. Not to be limited by theory, a mutation in at least some extracellular-transmembrane domains of CLEs provided herein, are responsible for signaling of the CLE in the absence of ligand, by bringing activating chains together that are not normally together, or by changing the confirmation of a linked transmembrane and/or intracellular domain.

Exemplary extracellular and transmembrane domains for CLEs of embodiments that include such domains, in illustrative embodiments, are extracellular regions, typically less than 30 amino acids of the membrane-proximal extracellular domains along with transmembrane domains from mutant receptors that have been reported to be constitutive, that is not require ligand binding for activation of an associated intracellular domain. In illustrative embodiments, such extracellular and transmembrane domains include IL7RA Ins PPCL, CRLF2 F232C, CSF2RB V449E, CSF3R T640N, EPOR L251C I252C, GHR E260C 1270C, IL27RA F523C, and MPL S505N. In some embodiments, the extracellular and transmembrane domain does not comprise more than 10, 20, 25 30 or 50 consecutive amino acids that are identical in sequence to a portion of the extracellular and/or transmembrane domain of IL7RA, or a mutant thereof. In some embodiments, the extracellular and transmembrane domain is other than IL7RA Ins PPCL. In some embodiments, the extracellular and transmembrane does not comprise more than 10, 20, 25, 30, or 50 consecutive amino acids that are identical in sequence to a portion of the extracellular and/or transmembrane domain of IL15R.

In embodiments of any of these aspects and embodiments wherein the transmembrane domain is a type I transmembrane protein, the transmembrane domain can be a Type I growth factor receptor, a hormone receptor, a T cell receptor, or a TNF-family receptor. In an embodiment of any of the aspects and embodiments wherein the chimeric polypeptide comprises an extracellular domain and wherein the extracellular domain comprises a dimerizing motif, the transmembrane domain can be a Type I cytokine receptor, a hormone receptor, a T cell receptor, or a TNF-family receptor.

In some embodiments, the extracellular and transmembrane domain is the viral protein LMP1, or a mutant and/or fragment thereof. LMP1 is a multispan transmembrane protein that is known to activate cell signaling independent of ligand when targeted to lipid rafts or when fused to CD40 (Kaykas et al. EMBO J. 20: 2641 (2001)). A fragment of LMP1 is typically long enough to span a plasma membrane and to activate a linked intracellular domain(s). For example, the LMP1 can be between 15 and 386, 15 and 200, 15 and 150, 15 and 100, 18 and 50, 18 and 30, 20 and 200, 20 and 150, 20 and 50, 20 and 30, 20 and 100, 20 and 40, or 20 and 25 amino acids. A mutant and/or fragment of LMP1 when included in a CLE provided herein, retains its ability to activate an intracellular domain Furthermore, if present, the extracellular domain includes at least 1, but typically at least 4 amino acids and is typically linked to another functional polypeptide, such as a clearance domain, for example, an eTag. In some embodiments, the lymphoproliferative element comprises an LMP1 transmembrane domain. In illustrative embodiments, the lymphoproliferative element comprises an LMP1 transmembrane domain and the one or more intracellular domains do not comprise an intracellular domain from TNFRSF proteins (i.e. CD40, 4-IBB, RANK, TACI, OX40, CD27, GITR, LTR, and BAFFR), TLR1 to TLR13, integrins, FcyRIII, Dectin1, Dectin2, NOD1, NOD2, CD16, IL-2R, Type I II interferon receptor, chemokine receptors such as CCR5 and CCR7, G-protein coupled receptors, TREM1, CD79A, CD79B, Ig-alpha, IPS-1, MyD88, RIG-1, MDA5, CD3Z, MyD88ΔTIR, TRIF, TRAM, TIRAP, MAL, BTK, RTK, RAC1, SYK, NALP3 (NLRP3), NALP3ΔLRR, NALP1, CARD9, DAI, IPAG, STING, Zap70, or LAT.

In other embodiments of CLEs provided herein, the extracellular domain includes a dimerizing moiety. Many different dimerizing moieties disclosed herein can be used for these embodiments. In illustrative embodiments, the dimerizing moieties are capable of homodimerizing. Not to be limited by theory, dimerizing moieties can provide an activating function on intracellular domains connected thereto via transmembrane domains. In some embodiments, a lymphoproliferative element provided herein comprises an extracellular domain, and in illustrative embodiments, the extracellular domain comprises a dimerizing motif. In illustrative embodiments of this aspect, the extracellular domain comprises a leucine zipper. In some embodiments, the leucine zipper is from a jun polypeptide, for example c-jun. In certain embodiments the c-jun polypeptide is the c-jun polypeptide region of ECD-11.

An extracellular domain with a dimerizing moiety can also serve a function of connecting a cell tag polypeptide to a cell expressing a CLE. In some embodiments, the dimerizing agent can be located intracellularly rather than extracellularly. In some embodiments, more than one or multiples of dimerizing domains can be used. In any aspects or embodiments wherein the extracellular domain of a CLE comprises a dimerizing motif, the dimerizing motif can be selected from the group consisting of: a leucine zipper motif-containing polypeptide, CD69, CD71, CD72, CD96, Cd105, Cd161, Cd162, Cd249, CD271, and Cd324, as well as mutants and/or active fragments thereof that retain the ability to dimerize. In any of the aspects and embodiments herein wherein the extracellular domain of a CLE comprises a dimerizing motif, the dimerizing motif can require a dimerizing agent, and the dimerizing motif and associated dimerizing agent can be selected from the group consisting of: FKBP and rapamycin or analogs thereof, GyrB and coumermycin or analogs thereof, DHFR and methotrexate or analogs thereof, or DmrB and AP20187 or analogs thereof, as well as mutants and/or active fragments of the recited dimerizing proteins that retain the ability to dimerize. In some aspects and illustrative embodiments, a lymphoproliferative element is constitutively active, and is other than a lymphoproliferative element that requires a dimerizing agent for activation.

Internally dimerizing and/or multimerizing lymphoproliferative elements in one embodiment are an integral part of a system that uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs and a myristoylation sequence to the cytoplasmic signaling domain of a target receptor, one can stimulate signaling in a dimerizer drug-dependent, but ligand and ectodomain-independent manner This provides the system with temporal control, reversibility using monomeric drug analogs, and enhanced specificity. The high affinity of third-generation AP20187/AP1903 dimerizer drugs for their binding domain, FKBP12 permits specific activation of the recombinant receptor in vivo without the induction of non-specific side effects through endogenous FKBP12. FKBP12 variants having amino acid substitutions and deletions, such as FKBP12V36, that bind to a dimerizer drug, may also be used. In addition, the synthetic ligands are resistant to protease degradation, making them more efficient at activating receptors in vivo than most delivered protein agents.

Extracellular domains for embodiments where extracellular domains have a dimerizing motif, are long enough to form dimers, such as leucine zipper dimers. As such, extracellular domains that include a dimerizing moiety can be from 15 to 100, 20 to 50, 30 to 45, or 35 to 40 amino acids, of in illustrative embodiments is a c-Jun portion of a c-Jun extracellular domain Extracellular domains of polypeptides that include a dimerizing moiety, may not retain other functionalities. For example, for leucine zippers embodiments, such leucine zippers are capable of forming dimers because they retain a motif of leucines spaced 7 residues apart along an alpha helix. However, leucine zipper moieties of certain embodiments of CLEs provided herein, may or may not retain their DNA binding function.

A spacer of between 1 and 4 alanine residues can be included in CLEs between the extracellular domain that has a dimerizing moiety, and the transmembrane domain. Not to be limited by theory, it is believed that the alanine spacer affects signaling of intracellular domains connected to the leucine zipper extracellular region via the transmembrane domain, by changing the orientation of the intracellular domains.

In illustrative embodiments, CLEs include a cell tag domain Details regarding cell tags are provided in other sections herein. Any of the cell tags provided herein can be part of a CLE. Typically, the cell tag is linked to the N terminus of the extracellular domain Not to be limited by theory, in some embodiments, the extracellular domain includes the function of providing a linker, in illustrative embodiments a flexible linker, linking a cell tag domain to a cell that expresses the CLE.

Furthermore, polynucleotides that include a nucleic acid sequence encoding a CLE provided herein, also typically comprise a signal sequence to direct expression to the plasma membrane. Exemplary signal sequences are provided herein in other sections. Elements can be provided on the transcript such that both a CAR and CLE are expressed from the same transcript in certain embodiments.

### Binding and Fusogenic Elements

Many of the methods, compositions, and kits provided herein include retroviral particles with envelope proteins on their surface, for example, multiple copies of a T cell and/or NK cell binding polypeptide and multiple copies of a fusogenic polypeptide, also called a fusogen. A “binding polypeptide” includes one or more polypeptides, typically glycoproteins, that identify and bind the target host cell. A “fusogenic polypeptide” mediates fusion of the retroviral and target host cell membranes, thereby allowing a retroviral genome to enter the target host cell. In certain embodiments, the binding polypeptide(s) and the fusogenic polypeptide(s) are on the same envelope protein, e.g., a heterologous glycoprotein. In other embodiments, the binding polypeptide(s) and the fusogenic polypeptide(s) are on two or more different heterologous glycoproteins.

One or both of these binding and fusogenic polypeptide functions can be provided by a pseudotyping element. In some embodiments, the binding polypeptide function can be performed by an activation element, as disclosed elsewhere herein. The pseudotyping of replication incompetent recombinant retroviral particles with heterologous envelope glycoproteins typically alters the tropism of a virus and facilitates the transduction of host cells. In some embodiments provided herein, pseudotyping elements are provided as polypeptide(s)/protein(s), or as nucleic acid sequences encoding the polypeptide(s)/protein(s).

In some embodiments, the pseudotyping element comprises the envelope protein from a different virus. In some embodiments, the pseudotyping element is the feline endogenous virus (RD114) envelope protein, an oncoretroviral amphotropic envelope protein, an oncoretroviral ecotropic envelope protein, the vesicular stomatitis virus envelope protein (VSV-G) (SEQ ID NO: 336), the baboon retroviral envelope glycoprotein (BaEV) (SEQ ID NO: 337), the murine leukemia envelope protein (MuLV) (SEQ ID NO: 338), the influenza glycoprotein HA surface glycoprotein (HA), the influenza glycoprotein neurominidase (NA), the paramyxovirus Measles envelope protein H, the paramyxovirus Measles envelope protein F, the Tupaia paramyxovirus (TPMV) envelope protein H, the TPMV envelope protein F, glycoproteins G and F from the Henipavirus genus, the Nipah virus (NiV) envelope protein F, the NiV envelope protein G, the Sindbis virus (SINV) protein E1, the SINV protein E2, and/or functional variants or fragments of any of these envelope proteins (see, e.g. Frank and Bucholz Mol Ther Methods Clin Dev. 2018 Oct. 17;12:19-31).

In some embodiments, the pseudotyping element can be wild-type BaEV. Not to be limited by theory, BaEV contains an R peptide that has been shown to inhibit transduction. In some embodiments, the BaEV can contain a deletion of the R peptide. In some embodiments, the BaEV can contain a deletion of the inhibitory R peptide after the nucleotides encoding the amino acid sequence HA, referred to herein as BaEVΔR (HA) (SEQ ID NO: 339). In some embodiments, the BaEV can contain a deletion of the inhibitory R peptide after the nucleotides encoding the amino acid sequence HAM, referred to herein as BaEVΔR (HAM) (SEQ ID NO: 340).

In some embodiments, the pseudotyping element can be wild-type MuLV. In some embodiments, the MuLV can contain one or more mutations to remove the furin-mediated cleavage site located between the transmembrane (TM) and surface (SU) subunits of the envelope glycoprotein. In some embodiments the MuLV contains the SUx mutation (MuLVSUx) (SEQ ID NO:372) which inhibits furin-mediated cleavage of MuLV envelope protein in packaging cells. In certain embodiments the C-terminus of the cytoplasmic tail of the MuLV or MuLVSUx protein is truncated by 4 to 31 amino acids. In certain embodiments the C-terminus of the cytoplasmic tail of the MuLV or MuLVSUx protein is truncated by 4, 8, 12, 16, 20, 24, 28, or 31 amino acids.

In some embodiments, the pseudotyping elements include a binding polypeptide and a fusogenic polypeptide derived from different proteins. In one aspect, the pseudotyping element can comprise an influenza protein hemagglutinin HA and/or a neuraminidase (NA). In certain embodiments the HA is from influenza A virus subtype H1N1. In illustrative embodiments the HA is from H1N1 PR8 1934 in which the monobasic trypsin-dependent cleavage site has been mutated to a more promiscuous multibasic sequence (SEQ ID NO:311). In certain embodiments the NA is from influenza A virus subtype H10N7. In illustrative embodiments the NA is from H10N7-HKWF446C-07 (SEQ ID NO:312). In some embodiments, the binding polypeptide can be a functional variant or fragment of VSV-G, BaEV, BaEVΔR (HA), BaEVΔR (HAM), MuLV, MuLVSUx, influenza HA, influenza NA, or Measles envelope protein H that retains the ability to bind to a target cell, and the fusogenic polypeptide can be a functional variant or fragment of VSV-G, BaEV, BaEVΔR (HA), BaEVΔR (HAM), MuLV, MuLVSUx, influenza HA, influenza NA, or Measles envelope protein F that retains the ability to mediate fusion of the retroviral and target host cell membranes.

In another aspect, the replication incompetent recombinant retroviral particles of the methods and compositions disclosed herein can be pseudotyped with the fusion (F) and/or hemagglutinin (H) polypeptides of the measles virus (MV), as non-limiting examples, clinical wildtype strains of MV, and vaccine strains including the Edmonston strain (MV-Edm) (GenBank; AF266288.2) or fragments thereof. Not to be limited by theory, both hemagglutinin (H) and fusion (F) polypeptides are believed to play a role in entry into host cells wherein the H protein binds MV to receptors CD46, SLAM, and Nectin-4 on target cells and F mediates fusion of the retroviral and host cell membranes. In an illustrative embodiment, especially where the target cell is a T cell and/or NK cell, the binding polypeptide is a Measles Virus H polypeptide and the fusogenic polypeptide is a Measles Virus F polypeptide.

In some studies, lentiviral particles pseudotyped with truncated F and H polypeptides had a significant increase in titers and transduction efficiency (Funke et al. 2008. Molecular Therapy. 16(8):1427-1436), (Frecha et al. 2008. Blood. 112(13):4843-4852). The highest titers were obtained when the F cytoplasmic tail was truncated by 30 residues (referred to as MV(Ed)-FΔ30 (SEQ ID NO:313)). For the H variants, optimal truncation occurred when 18 or 19 residues were deleted (MV(Ed)-HΔ18 (SEQ ID NO:314) or MV(Ed)-HΔ19), although variants with a truncation of 24 residues with and without replacement of deleted residues with alanine (MV(Ed)-HΔ24 (SEQ ID NO:315) and MV(Ed)-HΔ24+A) also resulted in optimal titers. Accordingly, in some embodiments, including those directed to transducing T cells and/or NK cells, the replication incompetent recombinant retroviral particles of the methods and compositions disclosed herein are pseudotyped with mutated or variant versions of the measles virus fusion (F) and hemagglutinin (H) polypeptides, in illustrative examples, cytoplasmic domain deletion variants of measles virus F and H polypeptides. In some embodiments, the mutated F and H polypeptides are “truncated H” or “truncated F” polypeptides, whose cytoplasmic portion has been truncated, i e amino acid residues (or coding nucleic acids of the corresponding nucleic acid molecule encoding the protein) have been deleted. “HΔY” and “FΔX” designate such truncated H and F polypeptide, respectively, wherein “Y” refers to 1-34 residues that have been deleted from the amino termini and “X” refers to 1-35 residues that have been deleted from the carboxy termini of the cytoplasmic domains. In a further embodiment, the “truncated F polypeptide” is FΔ24 or FΔ30 and/or the “truncated H protein” is selected from the group consisting of HΔ14, HΔ15, HΔ16, HΔ17, HΔ18, HΔ19, HΔ20, HΔ21+A, HΔ24 and HΔ24+4A, more preferably HΔ18 or HΔ24. In an illustrative embodiment, the truncated F polypeptide is MV(Ed)-FΔ30 and the truncated H polypeptide is MV(Ed)-HΔ18.

In some embodiments, the pseudotyping elements can be the envelope proteins from the Henipavirus genus (e.g. Nipah, Hendra, Cedar, Mojiang or Kumasi virus) and include envelope glycoprotein G (Henipavirus-G protein) and their fusion partner envelope glycoprotein F (Henipavirus-F protein). In some embodiments, the Henipavirus-F protein comprises the sequence of SEQ ID NO:374 and the Henipavirus-G protein comprises the sequence of SEQ ID NO:375. In some embodiments, the Henipavirus-F protein comprises a sequence that has at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:374. In some embodiments, the Henipavirus-G protein comprises a sequence that has at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:375.

In some embodiments, the Henipavirus-G protein can contain one or more mutations to modify (e.g., truncate) the cytoplasmic tail and thus improve pseudotyping and particle incorporation efficiency (Palomares et al. 2013. J Virol. 87(8):4794-4794; Witting et al. 2013. Gene Ther. 20(10):997-1005; Bender et al. 2016. Plos Pathog. 12(6): e1005641). In certain embodiments, the N-terminus of the cytoplasmic tail of any of the Henipavirus-G proteins can be truncated by 1 to all of its amino acids. In some embodiments, the residues of the Henipavirus-G protein involved in receptor binding are mutated to alter, and in illustrative embodiments remove, their native interactions with their natural receptors. In certain embodiments, the Henipavirus-G protein is mutated for example, but not limited to, at one or more of Y389, E501, W504, E505, V507, Q530, E533, or I588 of SEQ ID NO:375 (amino acids are given for Nipah-G, also referred to as NiV-G, and a skilled artisan will be able to identify the corresponding glutamines of other Henipavirus-G proteins) (Guillaume et al. 2006. J Virol. 80(15):7546-7554; Negrete et al. 2007. J Virol. 81(19):10804-10814; Xu et al. 2008. P Natl Acad Sci USA. 105(29):9953-9958; Xu et al. 2012. Plos One. 7(11): e48742; Bender et al. 2016. Plos Pathog. 12(6): e1005641). In some embodiments, Henipavirus-G protein is SEQ ID NO:375 with mutations E533A and/or Q530A. In some embodiments, one or more N- or O-glycosylation sites are mutated to improve pseudotyping and fusion (Biering et al. 2012. J Virol. 86(22):11991-12002; Stone et al. 2016. Plos Pathog. 12(2): e1005445). In some embodiments, one or more N-glycosylation sites are mutated for example, but not limited to, at one or more of N72, N159, N306, N378, N417, N481, or N529 of SEQ ID NO:375, or the corresponding glutamines of other Henipavirus-G proteins, to another amino acid such as glutamine. In some embodiments, one or more O-glycosylation sites are mutated from serine or threonine to another amino acid such as alanine. In some embodiments, one or more of the serine or threonine residues in the heavily O-glycosylated stalk domain from amino acids 103 to 137 of SEQ ID NO:375, is mutated to, for example, alanine. In other embodiments, the C-terminus of the Henipavirus-G protein can be modified and fused to a binding polypeptide and in illustrative embodiments, an activation element, such as an antibody or antibody mimetic, which in illustrative embodiments can be an anti-CD3 antibody or antibody mimetic (Bender et al. 2016. Plos Pathog. 12(6): e1005641; Jamali et al. 2019. Mol Ther-Meth Clin D. 13:371-379; Frank et al. 2020. Blood Adv. 4(22):5702-5715) .

In some embodiments, the F proteins can contain one or more mutations to modify (e.g., truncate) the cytoplasmic tail and thus improve pseudotyping, particle incorporation efficiency, and/or cleavage of the F protein from the inactive F0 to the cleaved active F1 form (Khetawat et al. 2010. Virol J. 7:312; Palomares et al. 2013. J Virol. 87(8):4794-4794; Witting et al. 2013. Gene Ther. 20(10):997-1005; Bender et al. 2016. Plos Pathog. 12(6): e1005641; Johnston et al. 2017. J Virol. 91(10): e02150-16). In some embodiments, one or more N-glycosylation sites are mutated for example, but not limited to, at one or more of N64, N67, N99, N414, or N 464 to another amino acid such as glutamine. In certain embodiments, the C-terminus of the cytoplasmic tail of the envelope glycoprotein F from the Henipavirus genus (Henipavirus-F protein) is truncated by 1 to all of its amino acids. In some embodiments, the F protein can contain one or more mutations to make it more fusogenic (Aguilar et al. 2007. J Virol. 81(9):4520-4532; Weis et al. 2015. Eur J Cell Biol. 94(7-9):316-322).

In some embodiments, the pseudotyping element can include a Henipavirus-F protein and a Henipavirus-G protein from the same virus of the Henipavirus genus (i.e., homologous proteins). In some embodiments, the pseudotyping element can include a Henipavirus-F protein and a Henipavirus-G protein from different viruses of the Henipavirus genus (i.e., heterologous proteins). In some embodiments, the pseudotyping element can include a Henipavirus-F protein and a Henipavirus-G protein can be chimeras composed of domains of heterologous proteins (Bradel-Tretheway et al. 2019. J Virol. 93(13): e00577-19).

In some embodiments, any of the pseudotyping elements can contain one or more mutations to modify (e.g., truncate) the cytoplasmic tail and thus improve pseudotyping, and particle incorporation efficiency. In certain embodiments, the N-terminus of the cytoplasmic tail is truncated by 1 to all of its amino acids. In some embodiments, the residues involved in receptor binding are mutated to alter, and in illustrative embodiments remove, their native interactions with their natural receptors. Similar to the mutations for Nipah-G protein, in some embodiments, the VSV-G protein is mutated for example, but not limited to, in the residues K47 or R354, for example K47A or K47Q and/or R354A or R354Q. In some embodiments, these pseudotyping elements are fused to heterologous binding polypeptides that function to direct or redirect the pseudotyping element to a new target protein on the same or different cell target.

In some embodiments, the separate binding and/or fusogenic polypeptides comprise one or more non virally-derived proteins. In some embodiments the binding polypeptide comprises an antibody, a ligand, or a receptor that binds a polypeptide on the target cell. In some embodiments, the binding polypeptide comprises an alternative non-antibody scaffold, also referred to herein as an antibody mimetic. In any of the aspects or embodiments provided herein that include a binding polypeptide, the binding polypeptide can be an antibody mimetic. In any of the aspects or embodiments provided herein that include a binding polypeptide that is an antibody, a suitable antibody mimetic can be used instead of the antibody. In some embodiments, the antibody mimetic can be an affibody, an afflilin, an affimer, an affitin, an alphabody, an alphamab, an anticalin, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain In some embodiments the binding polypeptide recognizes a protein on the surface of NK cells such as CD16, CD56, and CD57. In some embodiments the binding polypeptide recognizes a protein on the surface of T cells such as CD3, CD4, CD8, CD25, CD28, CD62L, CCR7, TCRa, and TCRb. In some embodiments, the binding polypeptide is also the activation element. In some embodiments, the binding polypeptide is a membrane polypeptide that binds CD3. In some embodiments, the fusogen is derived from the Sindbis virus glycoprotein that is modified to remove its binding activity, SV1, and the binding polypeptide is a membrane-bound anti-CD3 antibody (Yang et al. 2009. Pharm Res 26(6):1432-1445).

In some embodiments, the viral particles are copseudotyped with envelope glycoproteins from 2 or more heterologous viruses. In some embodiments, the viral particles are copseudotyped with VSV-G, or a functional variant or fragment thereof, and an envelope protein from RD114, BaEV, MuLV, influenza virus, measles virus, and/or a functional variant or fragment thereof. In some embodiments, the viral particles are copseudotyped with VSV-G and the MV(Ed)-H glycoprotein or the MV(Ed)-H glycoprotein with a truncated cytoplasmic domain In illustrative embodiments, the viral particles are copseudotyped with VSV-G and MV(Ed)-HΔ24. In certain embodiments, VSV-G is copseudotyped with MuLV or MuLV with a truncated cytoplasmic domain In other embodiments, VSV-G is copseudotyped with MuLVSUx or MuLVSUx with a truncated cytoplasmic domain In further illustrative embodiments, VSV-G is copseudotyped with a fusion of an antiCD3scFv to MuLV.

In some embodiments, the fusogenic polypeptide is derived from a class I fusogen. In some embodiments, the fusogenic polypeptide is derived from a class II fusogen. In some embodiments, both the binding polypeptide and the separate fusogenic polypeptide are virally-derived. In some embodiments, the fusogenic polypeptide includes multiple elements expressed as one polypeptide. In some embodiments, the binding polypeptide and fusogenic polypeptide are translated from the same transcript but from separate ribosome binding sites; in other embodiments, the binding polypeptide and fusogenic polypeptide are separated by a cleavage peptide site, which not to be bound by theory, is cleaved after translation, as is common in the literature, or a ribosomal skip sequence. In some embodiments, the translation of the binding polypeptide and fusogenic polypeptide from separate ribosome binding sites results in a higher amount of the fusogenic polypeptide as compared to the binding polypeptide. In some embodiments, the ratio of the fusogenic polypeptide to the binding polypeptide is at least 2:1, at least 3:1, at least 4:1, at least 5:1, at least 6:1, at least 7:1, or at least 8:1. In some embodiments, the ratio of the fusogenic polypeptide to the binding polypeptide is between 1.5:1, 2:1, or 3:1, on the low end of the range, and 3:1, 4:1, 5:1, 6:1, 7:1, 8:1. 9:1 or 10:1 on the high end of the range.

In embodiments disclosed herein including short contacting times, many of the modified lymphocytes in a cell formulation have pseudotyping elements on their surfaces during reintroduction of the modified lymphocytes into the subject, either through association with the replication incompetent recombinant retroviral particle or by fusion of the retroviral envelopes with the plasma membranes of the modified lymphocytes. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the cell formulation can include a pseudotyping element on their surfaces. In some embodiments, the pseudotyping element can be bound to the surface of the modified lymphocytes and/or the pseudotyping element can be present in the plasma membrane of the modified lymphocytes.

### Activation Elements

Many of the methods and composition aspects of the present disclosure that include a replication incompetent recombinant retroviral particle further include an activation element, also referred to herein as a T cell activation element, or a nucleic acid encoding an activation element. Activation elements are envelope proteins of the replication incompetent recombinant retroviral particles. Cells of the immune system such as T lymphocytes recognize and interact with specific antigens through receptors or receptor complexes which, upon recognition or an interaction with such antigens, cause activation of the cell and expansion in the body. An example of such a receptor is the antigen-specific T lymphocyte receptor complex (TCR/CD3) expressed on the surface of T lymphocytes. The TCR recognizes antigenic peptides that are presented to it by the proteins of the major histocompatibility complex (MHC) on the surface of antigen presenting cells and other T lymphocyte targets. Stimulation of the TCR/CD3 complex results in activation of the T lymphocyte and a consequent antigen-specific immune response. Thus, activation elements provided herein, activate T cells by binding to one or more components of the T cell receptor associated complex, for example by binding to CD3. In some embodiments, the activation element can activate alone. In other cases, the activation requires activation through the TCR receptor complex in order to further activate cells. T lymphocytes also require a second, co-stimulatory signal to become fully active in vivo. Without such a signal, T lymphocytes are either non-responsive to antigen binding to the TCR, or become anergic. However, the second, co-stimulatory signal is not required for the transduction and expansion of T cells and can be provided, for example, by a later co-stimulatory signal from a CAR or LE after transduction, as provided elsewhere herein. In some embodiments, the co-stimulatory signal can be provided during transduction by, for example, CD28, a T lymphocyte protein, which interacts with CD80 and CD86 on antigen-producing cells.

Activation of the T cell receptor (TCR) CD3 complex and co-stimulation with CD28 can occur by ex vivo exposure to solid surfaces (e.g., beads) coated with anti-CD3 and anti-CD28. In some embodiments of the methods and compositions disclosed herein, resting T cells are activated by exposure to solid surfaces coated with anti-CD3 and anti-CD28 ex vivo. In other embodiments, resting T cells or NK cells, and in illustrative embodiments resting T cells, are activated by exposure to soluble anti-CD3 antibodies (e.g., at 50-150, or 75-125, or 100 ng/ml). In such embodiments, which can be part of methods for modifying, genetically modifying or transducing, in illustrative embodiments without prior activation, such activation and/or contacting can be carried out by including anti-CD3 in a transduction reaction mixture and contacting with optional incubating for any of the times provided herein. Furthermore, such activation with soluble anti-CD3 can occur by incubating lymphocytes, such as PBMCs, and in illustrative embodiments NK cells and in more illustrative embodiments, T cells, after they are contacted with retroviral particles in a media containing an anti-CD3. Such incubation can be for example, for between 5, 10, 15, 30, 45, 60, or 120 minutes on the low end of the range, and 15, 30, 45, 60, 120, 180, or 240 minutes on the high end of the range, for example, between 15 and 1 hours or 2 hours.

In certain illustrative embodiments of the methods, kits, and compositions provided herein, for example for modifying, genetically modifying, and/or transducing lymphocytes, especially T cells and/or NK cells, polypeptides that are capable of binding to an activating T cell surface protein are presented as “activation elements” on the surface of replication incompetent recombinant retroviral particles. Thus, in some embodiments, an activation element can perform the binding polypeptide function. In some embodiments, the activation element is an envelope protein. Such T cell and/or NK cell activation elements on the surface of a retroviral particle are present in embodiments herein for modifying, genetically modifying, and/or transducing lymphocytes, for example wherein the retroviral particle has a genome that encodes a CAR, self-driving CAR, or LE. In some embodiments, such retroviral particles whose surface has an activation element are used in methods and uses that include administration by subcutaneous administration, and in kit components for subcutaneous administration. The activation element function discussed herein this section, as well as the binding polypeptide and fusogenic polypeptide disclosed elsewhere herein, in certain illustrative embodiments are all found associated with the surface of a retroviral particle, as part of one, two, or three proteins, in illustrative embodiments glycoproteins, and in further illustrative embodiments, heterologous glycoproteins. For example, some activation element polypeptides, such as those that are capable of binding to CD3, can also provide a T cell binding polypeptide function.

In some embodiments, the activation element is a polypeptide capable of binding to a polypeptide on the surface of a lymphocyte, and in illustrative embodiments, a T cell and/or an NK cell. In illustrative embodiments, the activation element is capable of binding to a TCR complex polypeptide. In some embodiments, a TCR complex polypeptide is CD3D, CD3E, CD3G, CD3Z, TCRα, or TCRβ. In some embodiments, the activation element capable of binding to the TCR complex polypeptide is a polypeptide capable of binding to one or more of CD3D, CD3E, CD3G, CD3Z, TCRα, or TCRβ. In illustrative embodiments, the activation element activates ZAP-70. In some embodiments, the activation element includes a polypeptide capable of binding to CD16A, NKG2C, NKG2D, NKG2E, NKG2F, or NKG2H. In some embodiments, the polypeptide capable of binding to NKG2D is MIC-A, MIC-B, or a ULBP, for example ULBP1 or ULBP2. In further embodiments, the polypeptide capable of binding to CD16A includes capable of binding to one or more of NKp46, 2B4, CD2, DNAM, NKG2C, NKG2D, NKG2E, NKG2F, or NKG2H. In some embodiments, the activation element is a polypeptide capable of binding to one or more of the following combinations: NKp46 and 2B4, NKp46 and CD2, NKp46 and DNAM, NKp46 and NKG2D, 2B4 and DNAM, or 2B4 and NKG2D. In some embodiments, the activation element can be two or more polypeptides capable of binding to polypeptides on the surface of a lymphocyte. In some embodiments, the activation element can be one or more polypeptides capable of binding to at least one of the following combinations: NKp46 and 2B4, NKp46 and CD2, NKp46 and DNAM, NKp46 and NKG2D, 2B4 and DNAM, or 2B4 and NKG2D. In illustrative embodiments the activation element is a polypeptide capable of binding to CD3E. In some embodiments, the polypeptide capable of binding to CD3 is an anti-CD3 antibody, or a fragment thereof that retains the ability to bind to CD3. In illustrative embodiments, the anti-CD3 antibody or fragment thereof is a single chain anti-CD3 antibody, such as but not limited to, an anti-CD3 scFv. In another illustrative embodiment, the polypeptide capable of binding to CD3 is anti-CD3scFvFc. In some embodiments, the activation element is an antibody. In some embodiments, the activation element comprises an alternative non-antibody scaffold, also referred to herein as an antibody mimetic. In any of the aspects or embodiments provided herein that include an activation element capable of binding to a polypeptide on the surface of a lymphocyte, and in illustrative embodiments, a T cell, the binding polypeptide can be an antibody mimetic. In some embodiments, the antibody mimetic can be an affibody, an afflilin, an affimer, an affitin, an alphabody, an alphamab, an anticalin, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain In any of the aspects or embodiments provided herein that include an activation element that is an antibody, a suitable antibody mimetic can be used instead of the antibody. In some embodiments, the activation element capable of binding a polypeptide on the surface of a lymphocyte (e.g. TCRβ is a superantigen polypeptide.

A number of anti-human CD3 monoclonal antibodies and antibody fragments thereof are available, and can be used in the present invention, including but not limited to UCHT1, OKT-3, HIT3A, TRX4, X35-3, VIT3, BMA030 (BW264/56), CLB-T3/3, CRIS7, YTH12.5, F111409, CLB-T3.4.2, TR-66, TR66.opt, HuM291, WT31, WT32, SPv-T3b, 11D8, XIII-141, XIII46, XIII-87, 12F6, T3/RW2-8C8, T3/RW24B6, OKT3D, M-T301, SMC2 and F101.01.

In other embodiments, the activation element on the surfaces of the replication incompetent recombinant retroviral particles can include one or more polypeptides capable of binding CD2, CD28, OX40, 4-1BB, ICOS, CD9, CD53, CD63, CD81, and/or CD82 and optionally one or more polypeptides capable of binding CD3. In illustrative embodiments, the activation element is a polypeptide capable of binding a mitogenic tetraspanin, for example, a polypeptide capable of binding to CD81, CD9, CD53, CD63, or CD82. In some embodiments, the activation element is a tetraspanin. Tetraspanins are known in the art. In some embodiments, the tetraspanin can be TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231/TALLA-1/A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1a, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), or TSPAN33. In some embodiments, the tetraspanin can be TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231/TALLA-1/A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1a, UPK1A), TSPAN22 (RDS, PRPH2), TSPAN23 (ROM1), TSPAN24 (CD151), TSPAN26 (CD37), TSPAN31 (SAS), TSPAN32 (TSSC6), or TSPAN33. In illustrative embodiments, the tetraspanin is TSPAN7 (CD231/TALLA-1/A15), TSPAN9 (NET-5), TSPAN24 (CD151), TSPAN27 (CD82), TSPAN28 (CD81), TSPAN29 (CD9), or TSPAN30 (CD63). In some embodiments, the activation element is a tetraspanin, and the tetraspanin is TSPAN25 (CD53), TSPAN27 (CD82), TSPAN28 (CD81), TSPAN29 (CD9), or TSPAN30 (CD63). In some embodiments, a tetraspanin is the only envelope protein. In some embodiments, a tetraspanin is a pseudotyping element comprising the binding polypeptide and the fusogenic element. In some embodiments, a tetraspanin is the activation element and the pseudotyping element. In illustrative embodiments, the tetraspanin that is the activation element and the pseudotyping element is TSPAN29 (CD9).

In some embodiments, one or typically more copies of one or more of these activation elements can be expressed on the surfaces of the replication incompetent recombinant retroviral particles as polypeptides separate and distinct from the pseudotyping elements. In some embodiments, the activation elements can be expressed on the surfaces of the replication incompetent recombinant retroviral particles as fusion polypeptides. In illustrative embodiments, the fusion polypeptides include one or more activation elements and one or more pseudotyping elements or one or more binding and/or fusogenic elements. In further illustrative embodiments, the fusion polypeptide includes anti-CD3, for example an anti-CD3scFv, or an anti-CD3scFvFc, and a viral envelope protein. In one example the fusion polypeptide is the OKT-3scFv fused to the amino terminal end of a viral envelope protein such as the MuLV envelope protein, as shown in Maurice et al. (2002). In some embodiments, the fusion polypeptide is UCHT1scFv fused to a viral envelope protein, for example the MuLV envelop protein (SEQ ID NO:341), the MuLVSUx envelope protein (SEQ ID NO:366), VSV-G (SEQ ID NO:367), or functional variants or fragments thereof, including any of the membrane protein truncations provided herein. In illustrative embodiments, especially for compositions and methods herein for transducing lymphocytes in whole blood, the fusion polypeptide does not include any blood protein (e.g., blood Factor (e.g., Factor X)) cleavage sites in the portion of the fusion protein that resides outside the retroviral particle. In some embodiments, the fusion constructs do not include any furin cleavage sites. Furin is a membrane bound protease expressed in all mammalian cells examined, some of which is secreted and active in blood plasma (See e.g., C. Fernandez et al. J. Internal. Medicine (2018) 284; 377-387). Mutations can be made to fusion constructs using known methods to remove such protease cleavage sites.

Polypeptides that bind CD3, CD28, OX40, 4-1BB, or ICOS are referred to as activation elements because of their ability to activate resting T cells. In certain embodiments, nucleic acids encoding such an activation element are found in the genome of a replication incompetent recombinant retroviral particle that contains the activating element on its surface. In illustrative embodiments, nucleic acids encoding an activation element are not found in the replication incompetent recombinant retroviral particle genome. In some embodiments, the nucleic acids encoding an activation element are found in the genome of a virus packaging cell.

In some embodiments, the activation element is a polypeptide capable of binding to CD28, for example, an anti-CD28 antibody or an anti-CD28 scFv antibody, or a fragment thereof that retains the ability to bind to CD28. In other embodiments, the polypeptide capable of binding to CD28 is CD80, CD86, or a functional fragment thereof that is capable of binding CD28 and inducing CD28-mediated activation of Akt, such as an external fragment of CD80. In some aspects herein, an external fragment of CD80 means a fragment that is typically present on the outside of a cell in the normal cellular location of CD80, that retains the ability to bind to CD28.

Anti-CD28 antibodies are known in the art and can include, as non-limiting examples, the monoclonal antibodies 9.3 (an IgG2a antibody), KOLT-2 (an IgG1 antibody), 15E8 (an IgG1 antibody), 248.23.2 (an IgM antibody), and EX5.3D10 (an IgG2a antibody).

In an illustrative embodiment, an activation element includes two polypeptides, a polypeptide capable of binding to CD3 and a polypeptide capable of binding to CD28.

In certain embodiments, the polypeptide capable of binding to CD3 or CD28 is an antibody, a single chain monoclonal antibody or an antibody fragment, for example a single chain antibody fragment. Accordingly, the antibody fragment can be, for example, a single chain fragment variable region (scFv), an antibody binding (Fab) fragment of an antibody, a single chain antigen-binding fragment (scFab), a single chain antigen-binding fragment without cysteines (scFabΔC), a fragment variable region (Fv), a construct specific to adjacent epitopes of an antigen (CRAb), or a single domain antibody (VH or VL).

In some embodiments, at least 10%, 15%, 20%, 25%, 30%, 35%, 40%, 45%, 50%, 55%, 60%, 65%, 70%, 75%, 80%, 85%, or 90% of the modified lymphocytes in a cell formulation can include a T cell activation element on their surfaces. In some embodiments, the T cell activation element can be bound to the surface of the modified lymphocytes through, for example, a T cell receptor, and/or the pseudotyping element can be present in the plasma membrane of the modified lymphocytes.

In any of the embodiments disclosed herein, an activation element, or a nucleic acid encoding the same, can include a dimerizing or higher order multimerizing motif. Dimerizing and multimerizing motifs are well-known in the art and a skilled artisan will understand how to incorporate them into the polypeptides for effective dimerization or multimerization. In illustrative embodiments, the polypeptide capable of binding to CD3 is anti-CD3scFvFc, which in some embodiments is considered an anti-CD3 with a dimerizing motif without any additional dimerizing motif, since anti-CD3scFvFc constructs are known to be capable of dimerizing without the need for a separate dimerizing motif.

In some embodiments, when present on the surface of replication incompetent recombinant retroviral particles, an activation element including a dimerizing motif can be active in the absence of a dimerizing agent. In some embodiments, the dimerizing or multimerizing motif, or a nucleic acid sequence encoding the same, can be an amino acid sequence from transmembrane polypeptides that naturally exist as homodimers or multimers. In some embodiments, the dimerizing or multimerizing motif, or a nucleic acid sequence encoding the same, can be an amino acid sequence from a fragment of a natural protein or an engineered protein. In one embodiment, the homodimeric polypeptide is a leucine zipper motif-containing polypeptide (leucine zipper polypeptide). For example, a leucine zipper polypeptide derived from c-JUN, non-limiting examples of which are disclosed related to chimeric lymphoproliferative elements (CLEs) herein. In some embodiments, these transmembrane homodimeric polypeptides can include CD69, CD71, CD72, CD96, Cd105, Cd161, Cd162, Cd249, CD271, Cd324, or active fragments thereof.

In some embodiments, when present on the surface of replication incompetent recombinant retroviral particles, an activation element including a dimerizing motif can be active in the presence of a dimerizing agent. In some embodiments, the dimerizing motif, and nucleic acid encoding the same, can include an amino acid sequence from transmembrane proteins that dimerize upon ligand (also referred to herein as a dimerizer or dimerizing agent) binding. In some embodiments, the dimerizing motif and dimerizer can include (where the dimerizer is in parentheses following the dimerizer-binding pair): FKBP and FKBP (rapamycin or its analog); GyrB and GyrB (coumermycin or its analog); DHFR and DHFR (methotrexate); or DmrB and DmrB (AP20187). As noted above, rapamycin can serve as a dimerizer. Alternatively, a rapamycin derivative or analog can be used (see, e.g., WO96/41865; WO 99/36553; WO 01/14387; and Ye et al (1999) Science 283:88-91). A coumermycin analog can be used (see, e.g., Farrar et al. (1996) Nature 383:178-181; and U.S. Pat. No. 6,916,846). Although some embodiments of lymphoproliferative elements include a dimerizing agent, in some aspects and illustrative embodiments, a lymphoproliferative element is constitutively active, and is other than a lymphoproliferative element that requires a dimerizing agent for activation.

In some embodiments, an activation element is fused to a heterologous signal sequence and/or a heterologous membrane attachment sequence or a membrane bound protein, all of which help direct the activation element to the membrane. In some embodiments, posttranslational lipid modification can occur via myristoylation, palmitoylation, or GPI anchorage. In some embodiments, the heterologous membrane attachment sequence is a GPI anchor attachment sequence. The heterologous GPI anchor attachment sequence can be derived from any known GPI-anchored protein. In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In illustrative embodiments, the heterologous GPI anchor attachment sequence is derived from Fc receptor FcγRIIIb (CD16b) or decay accelerating factor (DAF), otherwise known as complement decay-accelerating factor or CD55.

In some embodiments, one or more of the activation elements include a heterologous signal sequence to help direct expression of the activation element to the cell membrane. Any signal sequence that is active in the packaging cell line can be used. In some embodiments, the signal sequence is a DAF signal sequence. In illustrative embodiments, an activation element is fused to a DAF signal sequence at its N terminus and a GPI anchor attachment sequence at its C terminus.

In an illustrative embodiment, the activation element includes anti-CD3 scFvFc fused to a GPI anchor attachment sequence derived from CD14 and CD80 fused to a GPI anchor attachment sequence derived from CD16b; and both are expressed on the surface of a replication incompetent recombinant retroviral particle provided herein. In some embodiments, the anti-CD3 scFvFc is fused to a DAF signal sequence at its N terminus and a GPI anchor attachment sequence derived from CD14 at its C terminus and the CD80 is fused to a DAF signal sequence at its N terminus and a GPI anchor attachment sequence derived from CD16b at its C terminus; and both are expressed on the surface of a replication incompetent recombinant retroviral particle provided herein. In some embodiments, the DAF signal sequence includes amino acid residues 1-30 of the DAF protein.

In some embodiments, an activation element can be separate from the replication incompetent recombinant retroviral particle. Thus, in some embodiments, the replication incompetent recombinant retroviral particle do not comprise an activation element on their surface.

In some embodiments, more than one activation element is used. In some embodiments, the activation element can be a superantigen, for example lipopolysaccharide, SEC3, and Staphylococcal enterotoxin B. In some embodiments, the activation element can be a cytokine. In some embodiments, the activation element can be phorbol myristate acetate (PMA), ionomycin, or phytohemagglutinin (PHA). In some embodiments, the concentration of PMA in a cell formulation or to be administered separately from the replication incompetent recombinant retroviral particles can be 10, 25, 50, 75, or 100 ng/ml or between 10 and 100 ng/ml or 25 and 75 ng/ml. In some embodiments, the concentration of ionomycin in a cell formulation or to be administered separately from the replication incompetent recombinant retroviral particles can be at least or about 100, 250, 500, or 750 ng/ml or 1, 2, 3, 4, or 5 μg/ml or between 100 ng/ml and 5 μg/ml or between 500 ng/ml and 2 μg/ml. In some embodiments, the concentration of PHA in a cell formulation or to be administered separately from the replication incompetent recombinant retroviral particles can be at least or about 0.1 μg/ml, 0.25 μg/ml, 0.5 μg/ml, 1 μg/ml, 2.5 μg/ml, 5 μg/ml, 7.5 μg/ml, or 10 μg/ml or between 0.1 and 10 μg/ml, 1 and 10 μg/ml, or 2.5 and 7.5 μg/ml. In some embodiments, the activation element is administered within 5, 10, 15, 20, 30, 45, or 60 minutes, or 1, 2, 3, 4, 5, 6, 7, 8, 10, 12, 18, or 24 hours or 1, 2, 3, 4, 5, 6, 7, 14, 21, or 28 days of administering a cell formulation. In some embodiments, the activation element or elements are administered multiple times, for example on different days following administration of the cell formulation.

### Membrane-Bound Cytokines

Some embodiments of the method and composition aspects provided herein, include a membrane-bound cytokine, or polynucleotides encoding a membrane-bound cytokine. Cytokines are typically, but not always, secreted proteins. Cytokines that are naturally secreted can be engineered as fusion proteins to be membrane-bound. Membrane-bound cytokine fusion polypeptides are included in methods and compositions disclosed herein, and are also an aspect of the invention. In some embodiments, replication incompetent recombinant retroviral particles have a membrane-bound cytokine fusion polypeptide on their surface that is capable of binding a T cell and/or NK cell and promoting proliferation and/or survival thereof. Typically, membrane-bound polypeptides are incorporated into the membranes of replication incompetent recombinant retroviral particles, and when a cell is transduced by the replication incompetent recombinant retroviral particles, the fusion of the retroviral and host cell membranes results in the polypeptide being bound to the membrane of the transduced cell.

In some embodiments, the cytokine fusion polypeptide includes IL-2, IL-7, IL-15, or an active fragment thereof. The membrane-bound cytokine fusion polypeptides are typically a cytokine fused to heterologous signal sequence and/or a heterologous membrane attachment sequence. In some embodiments, the heterologous membrane attachment sequence is a GPI anchor attachment sequence. The heterologous GPI anchor attachment sequence can be derived from any known GPI-anchored protein (reviewed in Ferguson M A J, Kinoshita T, Hart G W. Glycosylphosphatidylinositol Anchors. In: Varki A, Cummings R D, Esko J D, et al., editors. Essentials of Glycobiology. 2nd edition. Cold Spring Harbor (N.Y.): Cold Spring Harbor Laboratory Press; 2009. Chapter 11). In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In an illustrative embodiment, the heterologous GPI anchor attachment sequence is derived from Fc receptor FcγRIIIb (CD16b). In some embodiments, the GPI anchor is the GPI anchor of DAF.

In illustrative embodiments, the membrane-bound cytokine is a fusion polypeptide of a cytokine fused to DAF. DAF is known to accumulate in lipid rafts that are incorporated into the membranes of replication incompetent recombinant retroviral particles budding from packaging cells. Accordingly, not to be limited by theory, it is believed that DAF fusion proteins are preferentially targeted to portions of membranes of packaging cells that will become part of a recombinant retroviral membrane.

In non-limiting illustrative embodiments, the cytokine fusion polypeptide is an IL-7, or an active fragment thereof, fused to DAF. In a specific non-limiting illustrative embodiment, the fusion cytokine polypeptide includes in order: the DAF signal sequence (residues 1-31 of DAF), IL-7 without its signal sequence, and residues 36-525 of DAF.

### Packaging Cell Lines/Methods of Making Recombinant Retroviral Particles

The present disclosure provides mammalian packaging cells and packaging cell lines that produce replication incompetent recombinant retroviral particles. The cell lines that produce replication incompetent recombinant retroviral particles are also referred to herein as packaging cell lines. A non-limiting example of such method is illustrated in WO2019/055946. Further exemplary methods for making retroviral particles are provided herein, for example in the Examples section herein. Such methods include, for example, a 4 plasmid system or a 5 plasmid system when a nucleic acid encoding an additional membrane bound protein, such as a T cell activation element that is not a fusion with the viral envelope, such as a GPI-linked anti-CD3, is included (See WO2019/05546). In an illustrative embodiment, provided herein is a 4 plasmid system in which a T cell activation element, such as a GPI-linked anti-CD3, is encoded on one of the packaging plasmids such as the plasmid encoding the viral envelope or the plasmid encoding REV, and optionally a second viral membrane-associated transgene such as a membrane bound cytokine can be encoded on the other packaging plasmid. In each case the nucleic acid encoding the viral protein is separated from the transgene by an IRES or a ribosomal skip sequence such as P2A or T2A. Such 4 plasmid system and associated polynucleotides as stated in the Examples, provided increased titers as compared to a 5 vector system in transient transfections, and thus provide illustrative embodiments herein. The present disclosure provides packaging cells and mammalian cell lines that are packaging cell lines that produce replication incompetent recombinant retroviral particles that genetically modify target mammalian cells and the target mammalian cells themselves. In illustrative embodiments, the packaging cell comprises nucleic acid sequences encoding a packageable RNA genome of the replication incompetent retroviral particle, a REV protein, a gag polypeptide, a pol polypeptide, and a pseudotyping element.

The cells of the packaging cell line can be adherent or suspension cells. Exemplary cell types are provided hereinbelow. In illustrative embodiments, the packaging cell line can be a suspension cell line, i.e., a cell line that does not adhere to a surface during growth. The cells can be grown in a chemically-defined media and/or a serum-free media. In some embodiments, the packaging cell line can be a suspension cell line derived from an adherent cell line, for example, the HEK293 cell line can be grown in conditions to generate a suspension-adapted HEK293 cell line according to methods known in the art. The packaging cell line is typically grown in a chemically defined media. In some embodiments, the packaging cell line media can include serum. In some embodiments, the packaging cell line media can include a serum replacement, as known in the art. In illustrative embodiments, the packaging cell line media can be serum-free media. Such media can be a chemically defined, serum-free formulation manufactured in compliance with Current Good Manufacturing Practice (CGMP) regulations of the US Food and Drug Administration (FDA). The packaging cell line media can be xeno-free and complete. In some embodiments, the packaging cell line media has been cleared by regulatory agencies for use in ex vivo cell processing, such as an FDA 510(k) cleared device.

Accordingly, in one aspect, provided herein is a method of making a replication incompetent recombinant retroviral particle including: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; and B. harvesting the replication incompetent recombinant retroviral particle from the serum-free media. In another aspect, provided herein is a method of transducing a lymphocyte with a replication incompetent recombinant retroviral particle comprising: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; B. harvesting the replication incompetent recombinant retroviral particle from the serum-free media; and C. contacting the lymphocyte with the replication incompetent recombinant retroviral particle, wherein the contacting is performed for less than 24 hours, 20 hours, 18 hours, 12 hours, 8 hours, 4 hours, 2 hours, 1 hour, 30 minutes, or 15 minutes (or between contacting and no incubation, or 15 minutes, 30 minutes, 1, 2, 3, or 4 hours on the low end of the range and 1, 2, 3, 4, 6, 8, 12, 18, 20, or 24 hours on the high end of the range), thereby transducing the lymphocyte.

The packageable RNA genome, in certain illustrative embodiments, is designed to express one or more target polypeptides, including as a non-limiting example, any of the engineered signaling polypeptides disclosed herein and/or one or more (e.g., two or more) inhibitory RNA molecules in opposite orientation (e.g., encoding on the opposite strand and in the opposite orientation), from retroviral components such as gag and pol. For example, the packageable RNA genome can include from 5′ to 3′: a 5′ long terminal repeat, or active truncated fragment thereof; a nucleic acid sequence encoding a retroviral cis-acting RNA packaging element; a nucleic acid sequence encoding a first and optionally second target polypeptide, such as, but not limited to, an engineered signaling polypeptide(s) in opposite orientation, which can be driven off a promoter in this opposite orientation with respect to the 5′ long terminal repeat and the cis-acting RNA packaging element, which in some embodiments is called a “fourth” promoter for convenience only (and sometimes referred to herein as the promoter active in T cells and/or NK cells), which is active in a target cell such as a T cell and/or an NK cell but in illustrative examples is not active in the packaging cell or is only inducibly or minimally active in the packaging cell; and a 3′ long terminal repeat, or active truncated fragment thereof. In some embodiments, the packageable RNA genome can include a central polypurine tract (cPPT)/central termination sequence (CTS) element. In some embodiments, the retroviral cis-acting RNA packaging element can be HIV Psi. In some embodiments, the retroviral cis-acting RNA packaging element can be the Rev Response Element. The engineered signaling polypeptide driven by the promoter in the opposite orientation from the 5′ long terminal repeat, in illustrative embodiments, is one or more of the engineered signaling polypeptides disclosed herein and can optionally express one or more inhibitory RNA molecules as disclosed in more detail herein and in WO2017/165245A2, WO2018/009923A1, and WO2018/161064A1. In some aspects, provided herein is a packageable RNA genome designed to express a self-driving CAR. Details regarding such replication incompetent recombinant retroviral particles, and composition and method aspects including a self-driving CAR, are disclosed in more detail herein, for example in the Self-Driving CAR Methods and Compositions section and in the Exemplary Embodiments section. In illustrative embodiments, the first one or more transcriptional units encoding a lymphoproliferative element is encoded in the reverse orientation and the second one or more transcriptional units encoding a CAR is in the forward orientation.

It will be understood that promoter number, such as a first, second, third, fourth, etc. promoter is for convenience only. A promoter that is called a “fourth” promoter should not be taken to imply that there are any additional promoters, such as first, second or third promoters, unless such other promoters are explicitly recited. It should be noted that each of the promoters are capable of driving expression of a transcript in an appropriate cell type and such transcript forms a transcription unit.

In some embodiments, the engineered signaling polypeptide can include a first lymphoproliferative element. Suitable lymphoproliferative elements are disclosed in other sections herein. As a non-limiting example, the lymphoproliferative element can be expressed as a fusion with a cell tag, such as an eTag, as disclosed herein. In some embodiments, the packageable RNA genome can further include a nucleic acid sequence encoding a second engineered polypeptide including a chimeric antigen receptor, encoding any CAR embodiment provided herein. For example, the second engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments where the target cell is a T cell, the promoter that is active in a target cell is active in a T cell, as disclosed elsewhere herein.

In some embodiments, the engineered signaling polypeptide can include a CAR, and the nucleic acid sequence can encode any CAR embodiment provided herein. For example, the engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments, the packageable RNA genome can further include a nucleic acid sequence encoding a second engineered polypeptide. In some embodiments, the second engineered polypeptide can be a lymphoproliferative element. In some embodiments where the target cell is a T cell or NK cell, the promoter that is active in a target cell is active in a T cell or NK cell, as disclosed elsewhere herein.

In some embodiments, the packageable RNA genome included in any of the aspects provided herein, can further include a riboswitch, as discussed in WO2017/165245A2, WO2018/009923A1, and WO2018/161064A1. In some embodiments, the nucleic acid sequence encoding the engineered signaling polypeptide can be in a reverse orientation with respect to the 5′ to 3′ orientation established by the 5′ LTR and the 3′ LTR. In further embodiments, the packageable RNA genome can further include a riboswitch and, optionally, the riboswitch can be in reverse orientation. In any of the embodiments disclosed herein, a polynucleotide including any of the elements can include a primer binding site. In illustrative embodiments, insulators and/or polyadenylation sequences can be placed before, after, between, or near genes to prevent or reduce unregulated transcription. In some embodiments, the insulator can be chicken HS4 insulator, Kaiso insulator, SAR/MAR elements, chimeric chicken insulator-SAR elements, CTCF insulator, the gypsy insulator, or the β-globin insulator or fragments thereof known in the art. In some embodiments, the insulator and/or polyadenylation sequence can be hGH polyA (SEQ ID NO:316), SPA1 (SEQ ID NO:317), SPA2 (SEQ ID NO:318), b-globin polyA spacer B (SEQ ID NO:319), b-globin polyA spacer A (SEQ ID NO:320), 250 cHS4 insulator v1 (SEQ ID NO:321), 250 cHS4 insulator v2 (SEQ ID NO:322), 650 cHS4 insulator (SEQ ID NO:323), 400 cHS4 insulator (SEQ ID NO:324), 650 cHS4 insulator and b-globin polyA spacer B (SEQ ID NO:325), or b-globin polyA spacer B and 650 cHS4 insulator (SEQ ID NO:326).

In any of the embodiments disclosed herein, a nucleic acid sequence encoding Vpx can be on the second or an optional third transcriptional unit, or on an additional transcriptional unit that is operably linked to the first inducible promoter.

Some aspects of the present disclosure include or are cells, in illustrative examples, mammalian cells, that are used as packaging cells to make replication incompetent recombinant retroviral particles, such as lentiviruses, for transduction of T cells and/or NK cells. In some aspects, provided herein are packaging cells to make replication incompetent recombinant retroviral particles that include a polynucleotide encoding a self-driving CAR. Details regarding such replication incompetent recombinant retroviral particles, and composition and method aspects including a self-driving CAR, are disclosed in more detail herein, for example in the Self-Driving CAR Methods and Compositions section and in the Exemplary Embodiments section.

Any of a wide variety of cells can be selected for in vitro production of a virus or virus particle, such as a redirected recombinant retroviral particle, according to the invention. Eukaryotic cells are typically used, particularly mammalian cells including human, simian, canine, feline, equine and rodent cells. In illustrative examples, the cells are human cells. In further illustrative embodiments, the cells reproduce indefinitely, and are therefore immortal. Examples of cells that can be advantageously used in the present invention include NIH 3T3 cells, COS cells, Madin-Darby canine kidney cells, human embryonic 293T cells and any cells derived from such cells, such as gpnlslacZ φNX cells, which are derived from 293T cells. Highly transfectable cells, such as human embryonic kidney 293T cells, can be used. By “highly transfectable” it is meant that at least about 50%, more preferably at least about 70% and most preferably at least about 80% of the cells can express the genes of the introduced DNA.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL1O), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, Hut-78, Jurkat, HL-60, and the like.

### Genetically Modified T Cells and NK Cells

In embodiments of the methods and compositions herein, genetically modified lymphocytes are produced, which themselves are a separate aspect of the invention. Such genetically modified lymphocytes can be genetically modified and/or transduced lymphocytes. In one aspect, provided herein a genetically modified T cell or NK cell is made using a method according to any aspect for genetically modifying T cells and/or NK cells in blood or a component thereof, provided herein. For example, in some embodiments, the T cell or NK cell has been genetically modified to express a first engineered signaling polypeptide. In illustrative embodiments, the first engineered signaling polypeptide can be a lymphoproliferative element or a CAR that includes an antigen-specific targeting region (ASTR), a transmembrane domain, and an intracellular activating domain In some embodiments, the T cell or NK cell can further include a second engineered signaling polypeptide that can be a CAR or a lymphoproliferative element. In some embodiments, the lymphoproliferative element can be a chimeric lymphoproliferative element. In some embodiments, the T cell or NK cell can further include a pseudotyping element on a surface. In some embodiments, the T cell or NK cell can further include an activation element on a surface. The CAR, lymphoproliferative element, pseudotyping element, and activation element of the genetically modified T cell or NK cell can include any of the aspects, embodiments, or subembodiments disclosed herein. In illustrative embodiments, the activation element can be anti-CD3 antibody, such as an anti-CD3 scFvFc.

In some embodiments, genetically modified lymphocytes are lymphocytes such as T cells or NK cells that have been genetically modified to express a first engineered signaling polypeptide comprising at least one lymphoproliferative element and/or a second engineered signaling polypeptide comprising a chimeric antigen receptor, which includes an antigen-specific targeting region (ASTR), a transmembrane domain, and an intracellular activating domain. In some embodiments of any of the aspects herein, the NK cells are NKT cells. NKT cells are a subset of T cells that express CD3 and typically coexpress an αβ T-cell receptor, but also express a variety of molecular markers that are typically associated with NK cells (such as NK1.1 or CD56).

Genetically modified lymphocytes of the present disclosure possess a heterologous nucleic acid sequence that has been introduced into the lymphocyte by a recombinant DNA method. For example, the heterologous sequence in illustrative embodiments is inserted into the lymphocyte during a method for transducing the lymphocyte provided herein. The heterologous nucleic acid is found within the lymphocyte and in some embodiments is or is not integrated into the genome of the genetically modified lymphocyte.

In illustrative embodiments, the heterologous nucleic acid is integrated into the genome of the genetically modified lymphocyte. Such lymphocytes are produced, in illustrative embodiments, using a method for transducing lymphocytes provided herein, that utilizes a recombinant retroviral particle. Such recombinant retroviral particle can include a polynucleotide that encodes a chimeric antigen receptor that typically includes at least an antigen-specific targeting region (ASTR), a transmembrane domain, and an intracellular activating domain. Provided herein in other sections of this disclosure are various embodiments of replication incompetent recombinant retroviral particles and polynucleotides encoded in a genome of the replication incompetent retroviral particle, that can be used to produce genetically modified lymphocytes that themselves form another aspect of the present disclosure.

Genetically modified lymphocytes of the present disclosure can be isolated outside the body. For example, such lymphocytes can be found in media and other solutions that are used for ex vivo transduction as provided herein. The lymphocytes can be present in a genetically unmodified form in blood that is collected from a subject in methods provided herein, and then genetically modified during method of transduction. The genetically modified lymphocytes can be found inside a subject after they are introduced or reintroduced into the subject after they have been genetically modified. The genetically modified lymphocytes can be a resting T cell or a resting NK cell, or the genetically modified T cell or NK cell can be actively dividing, especially after it expresses some of the functional elements provided in nucleic acids that are inserted into the T cell or NK cell after transduction as disclosed herein.

Provided herein in one aspect is a transduced and/or genetically modified T cell or NK cell, comprising a recombinant polynucleotide comprising one or more transcriptional units operatively linked to a promoter active in T cells and/or NK cells, in its genome.

In some aspects, provided herein are aspects that include a genetically modified and/or transduced T cell or NK cell that include a polynucleotide encoding a self-driving CAR. Details regarding such genetically modified and/or transduced T cells or NK cells, and composition and method aspects including a self-driving CAR, that contain such polynucleotides are disclosed in more detail herein, for example in the Self-Driving CAR Methods and Compositions section and in the Exemplary Embodiments section.

In some embodiments, provided herein are genetically modified lymphocytes, in illustrative embodiments T cells and/or NK cells, or self-driving CAR aspects provided herein, that relate to either aspects for transduction of T cells and/or NK cells in blood or a component thereof, that include transcription units that encode one, two, or more (e.g., 1-10, 2-10, 4-10, 1-6, 2-6, 3-6, 4-6, 1-4, 2-4, 3-4) inhibitory RNA molecules. In some embodiments, such inhibitory RNA molecules are lymphoproliferative elements and therefore, can be included in any aspect or embodiment disclosed herein as the lymphoproliferative element as long as they induce proliferation of a T cell and/or an NK cell, or otherwise meet a test for a lymphoproliferative element provided herein. In some embodiments, inhibitory RNA molecules directed against any of the targets identified in the Inhibitory RNA Molecules section herein.

In some embodiments of the aspect immediately above where the T cell or NK cell comprises one or more (e.g., two or more) inhibitory RNA molecules and the CAR, or nucleic acids encoding the same, the ASTR of the CAR is an MRB ASTR and/or the ASTR of the CAR binds to a tumor associated antigen. Furthermore, in some embodiments of the above aspect, the first nucleic acid sequence is operably linked to a riboswitch, which for example is capable of binding a nucleoside analog, and in illustrative embodiments is an antiviral drug such as acyclovir.

In the methods and compositions disclosed herein, expression of engineered signaling polypeptides is regulated by a control element, and in some embodiments, the control element is a polynucleotide comprising a riboswitch. In certain embodiments, the riboswitch is capable of binding a nucleoside analog and when the nucleoside analog is present, one or both of the engineered signaling polypeptides are expressed.

### Nucleic Acids

The present disclosure provides nucleic acid encoding polypeptides of the present disclosure and nucleic acids are disclosed for use in various methods herein. A nucleic acid will in some embodiments be DNA, including, e.g., a recombinant expression construct, or as all or part of the genome of a T cell or an NK cell, for example. A nucleic acid will in some embodiments be RNA, such as a retroviral genome or an expressed transcript within a packaging cell line, a T cell or an NK, for example. A nucleic acid will in some embodiments be RNA, e.g., in vitro synthesized RNA. In some embodiments, the nucleic acid can be isolated. As used herein, the term “isolated” means that the material is removed from its original environment (e.g., the natural environment if it is naturally occurring). For example, a naturally-occurring polynucleotide, or in other embodiments a polypeptide, present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. For example, an isolated nucleic can be part of recombinant nucleic acid vector, such as an expression vector, which in illustrative embodiments can be a replication incompetent recombinant retroviral particle. In some embodiments, the nucleic acid is manufactured in compliance with cGMP, as discussed herein for kit components.

In some embodiments, a nucleic acid provides for production of a polypeptide of the present disclosure, e.g., in a mammalian cell. In other cases, a subject nucleic acid provides for amplification of the nucleic acid encoding a polypeptide of the present disclosure.

For packaging viral particles, promoters suitable for use may be constitutive or inducible. For expression of the viral particle RNA, an LTR promoter or hybrid LTR promoter may be used. For example, RSV/LTR, TRE/LTR or LTR alone may be used to transcribe the nucleic acid to be packaged. Examples of LTR's include, but are not limited to, MSCV, GALV, HIV-1, HIV-2 and MuLV. In packaging lines containing the large T antigen, for example, incorporation of the SV40 origin of replication may be included in one or more packaging vectors to amplify circular plasmid DNA during transcription and/or translation. The use of multiple promoters may be utilized to prevent transcription factor competition. For example CMV, SV40, RSV, HSVTK, TRE and other promoters may be utilized to express different components of the LV particles. In some instances, viral particle components may be expressed from integrated expression vectors. In other cases, one or more of the nucleic acids may be introduced via transient expression. In some embodiments the use of inducible promoters are used to minimize cellular toxicity before viral particle packaging.

For expression of a transgene (e.g. a CAR) in a genetically modified cell, such as a lymphocyte, a macrophage, or a dendritic cell, suitable promoters include any constitutive promoter known in the art. In some embodiments, the constitutive promoter can be an EF1-a promoter, PGK promoter, CMV promoter, MSCV-U3 promoter (see, e.g., Jones et al., Human Gene Therapy (2009) 20: 630-40), SV40hCD43 promoter, VAV promoter, TCRβ promoter, UBC promoter, cytomegalovirus immediate early promoter, herpes simplex virus thymidine kinase promoter, early and late SV40 promoters, promoter present in long terminal repeats from a retrovirus, mouse metallothionein-I promoter, and various art-known tissue specific promoters. In some embodiments, a constitutive promoter can include the EF1-a promoter nucleotide sequence (SEQ ID NO:350), the PGK promoter nucleotide sequence (SEQ ID NO:351), or a functional portion or variant thereof. In some embodiments, a constitutive promoter can include other than the EF1-a promoter. In some embodiments, the promoter include light and/or heavy chain immunoglobulin gene promoter and enhancer elements.

In some embodiments, the promoter is not active in the packaging line or is only minimally active in the packaging line. Such embodiments have the advantage with expression an engineered T cell receptor or a CAR, that they would reduce, minimize, or in illustrative embodiments substantially eliminate, or even eliminate expression of the engineered T cell receptor or CAR in a encapsulated nucleic acid vector such as a RIR retroviral particle or a virus-like particle because of reduced, low, negligible, substantially no, or no expression of the engineered T cell receptor or CAR in the packaging cell line used to make the encapsulated nucleic acid vector. In illustrative embodiments, such expression is reduced, substantially eliminated, or eliminated on the surface of the encapsulated nucleic acid vector (e.g. RIR particle or virus-like particle). In some embodiments, the promoter can be a T cell-specific promoter, a CD8 cell-specific promoter, a CD4 cell-specific promoter, a NKT cell specific promoter, or an NK cell-specific promoter. In some embodiments, the T cell-specific promoter can be the CD3 zeta promoter or the CD3 delta promoter (see, e.g., Ji et al., J Biol Chem. 2002 Dec. 6; 277(49):47898-906). In illustrative embodiments, the T cell-specific promoter can be the CD3 zeta promoter. In some embodiments, the T cell-specific promoter can be a CD8 gene promoter. In some embodiments, the T cell-specific promoter can be a CD4 gene promoter (see, e.g., Salmon et al. (1993) Proc. Natl. Acad. Sci. USA 90:7739; and Marodon et al. (2003) Blood 101:3416). In some embodiments, an NK cell-specific promoter can be a Neri (p46) promoter (see, e.g., Eckelhart et al. (2011) Blood 117:1565). In some embodiments, the specific proteins encoded by the recombinant gene vector are not expressed, displayed, and/or incorporated in the surface of the gene vector (e.g. RIP). In some embodiments, this is accomplished by means of a T cell specific promoter driving transgene expression in the gene vector. In some embodiments, that promoter is a promoter from the CD3 family. In other embodiments it is a hybrid CD3 promoter. In other embodiments, the packaging cell line encodes a repressor protein capable of substantially suppressing the expression of the lentiviral transgenes in the packaging cell line. In some embodiments that suppressor may be a TET repressor protein. In yet other embodiments, the transcription factor activate against the protein in the packaging line has been suppressed or inactivated. In some embodiments the inactivation may be achieved through DNA editing nucleases. In other embodiments the inactivation is achieved through shRNA or miRNA. In other embodiments, the suppression of the transcription factor is achieved through a dominant negative protein or degron to the transcription factor. In other embodiments, the viral nucleic acids are controlled via a ligand inducible or repressible promoter not activated in the packaging cell line.

In other embodiments, the promoter can be a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like. Further discussion of suitable promoters for use in various methods and as separate aspects, are provided herein.

In some embodiments, the promoter is inducible in a cell to be genetically modified (e.g. CAR-T cell). In some embodiments, an inducible promoter can include a T cell-specific response element or an NFAT response element. In other embodiments the promoter may be regulated by an environmental condition, such as hypoxia, temperature, glucose, pH or light. In other embodiments the promoter may be responsive to concentrations of extracellular molecules. In some instances, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28: e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, Mass.), the disclosures of which are incorporated herein by reference.

In some aspects, provided herein are polynucleotides that include a promoter that is particularly useful for a self-driving CAR. Details regarding such promoters, and composition and method aspects including a self-driving CAR that contain such promoters, are disclosed in more detail herein, for example in the Self-Driving CAR Methods and Compositions section and in the Exemplary Embodiments section. In some cases, the promoter is a CD8 cell-specific promoter, a CD4 cell-specific promoter, a macrophage-specific promoter, or an NK-specific promoter. For example, a CD4 gene promoter can be used.

In some embodiments, e.g., for expression in a yeast cell, a suitable promoter is a constitutive promoter such as an ADH1, PGK1, ENO, or PYK1 promoter and the like; or a regulatable promoter such as a GALI, GAL1O, ADH2, PH05, CUP1, GAL7, MET25, MET3, CYC1, HIS3, ADH1, PGK, GAPDH, ADC1, TRP1, URA3, LEU2, ENO, TP1, or AOX1 promoter (e.g., for use in Pichia). Selection of the appropriate vector and promoter is well within the level of ordinary skill in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacterial., 1991: 173(1): 86-93; Alpuche-Aranda et al., PNAS, 1992; 89(21): 10079-83), a nirB promoter (Harborne et al. (1992) Mal. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mal. Microbial. 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Laci repressor protein changes conformation when contacted with lactose, thereby preventing the Laci repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25).

An isolated nucleotide sequence encoding a polypeptide of the disclosure can be present in a eukaryotic expression vector and/or a cloning vector. Nucleotide sequences encoding two separate polypeptides can be cloned in the same or separate vectors. An expression vector can include a selectable marker, an origin of replication, and other features that provide for replication and/or maintenance of the vector and expression of a transgene. For example, an expression vector typically includes a promoter operably linked to a transgene. Suitable expression vectors are known in the art and include, for example, plasmids and viral vectors. In some embodiments, the expression vector is a recombinant retroviral particle, as disclosed in detail herein.

Large numbers of suitable vectors and promoters are known to those of skill in the art; many are commercially available for generating subject recombinant constructs. The following bacterial vectors are provided by way of example: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). The following eukaryotic vectors are provided by way of example: pWLneo, pSV2cat, pOG44, PXR1, pSG (Stratagene) pSVK3, pBPV, pMSG, and pSVL (Pharmacia).

Expression vectors generally have convenient restriction sites located near the promoter sequence to provide for the insertion of nucleic acid sequences encoding heterologous proteins. A selectable marker operative in the expression host may be present.

As noted above, in some embodiments, a nucleic acid encoding a polypeptide of the present disclosure will in some embodiments be RNA, e.g., in vitro synthesized RNA. Methods for in vitro synthesis of RNA are known in the art; any known method can be used to synthesize RNA including a nucleotide sequence encoding a polypeptide of the present disclosure. Methods for introducing RNA into a host cell are known in the art. See, e.g., Zhao et al. (2010) Cancer Res. 15:9053. Introducing RNA including a nucleotide sequence encoding a polypeptide of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

Various aspects and embodiments that include a polynucleotide, a nucleic acid sequence, and/or a transcriptional unit, and/or a vector including the same, further include one or more of a Kozak-type sequence (also called a Kozak-related sequence herein), a woodchuck hepatitis virus post-transcriptional regulatory element (WPRE), and a double stop codon or a triple stop codon, wherein one or more stop codons of the double stop codon or the triple stop codon define a termination of a reading from of at least one of the one or more transcriptional units. In certain embodiments, a polynucleotide a nucleic acid sequence, and/or a transcriptional unit, and/or a vector including the same, further includes a Kozak-type sequence having a 5′ nucleotide within 10 nucleotides upstream of a start codon of at least one of the one or more transcriptional units. Kozak determined the Kozak consensus sequence, (GCC)GCCRCCATG (SEQ ID NO:327), for 699 vertebrate mRNAs, where R is a purine (A or G) (Kozak. Nucleic Acids Res. 1987 Oct. 26; 15(20):8125-48). In one embodiment the Kozak-type sequence is or includes CCACCAT/UG(G) (SEQ ID NO:328), CCGCCAT/UG(G) (SEQ ID NO:329), GCCGCCGCCAT/UG(G) (SEQ ID NO:330), or GCCGCCACCAT/UG(G) (SEQ ID NO:331) (with nucleotides in parenthesis representing optional nucleotides and nucleotides separated by a slash indicated different possible nucleotides at that position, for example depending on whether the nucleic acid is DNA or RNA. In these embodiments that include the AU/TG start codon, the A can be considered position 0. In certain illustrative embodiments, the nucleotides at −3 and +4 are identical, for example the −3 and +4 nucleotides can be G. In another embodiment the Kozak-type sequence includes an A or G in the 3rd position upstream of ATG where ATG is the start codon. In another embodiment the Kozak-type sequence includes an A or G in the 3rd position upstream of AUG where AUG is the start codon. In an illustrative embodiment, the Kozak sequence is (GCC)GCCRCCATG (SEQ ID NO:327), where R is a purine (A or G). In an illustrative embodiment, the Kozak-type sequence is GCCGCCACCAUG (SEQ ID NO:332). In another embodiment, which can be combined with the preceding embodiment that includes a Kozak-type sequence and/or the following embodiment that includes triple stop codon, the polynucleotide includes a WPRE element. WPREs have been characterized in the art (See e.g., (Higashimoto et al., Gene Ther. 2007; 14: 1298)) and as illustrated in WO2019/055946. In some embodiments, the WPRE element is located 3′ of a stop codon of the one or more transcriptional units and 5′ to a 3′ LTR of the polynucleotide. In another embodiment, which can be combined with either or both of the preceding embodiments (i.e. an embodiment wherein the polynucleotide includes a Kozak-type sequence and/or an embodiment wherein the polynucleotide includes a WPRE), the one or more transcriptional units terminates with one or more stop codons of a double stop codon or a triple stop codon, wherein the double stop codon includes a first stop codon in a first reading frame and a second stop codon in a second reading frame, or a first stop codon in frame with a second stop codon, and wherein the triple stop codon includes a first stop codon in a first reading frame, a second stop codon in a second reading frame, and a third stop codon in a third reading frame, or a first stop codon in frame with a second stop codon and a third stop codon.

A triple stop codon herein includes three stop codons, one in each reading frame, within 10 nucleotides of each other, and preferably having overlapping sequence, or three stop codons in the same reading frame, preferably at consecutive codons. A double stop codon means two stop codons, each in a different reading frame, within 10 nucleotides of each other, and preferably having overlapping sequences, or two stop codons in the same reading frame, preferably at consecutive codons.

In some of the methods and compositions disclosed herein, the introduction of DNA into PBMCs, B cells, T cells and/or NK cells and optionally the incorporation of the DNA into the host cell genome, is performed using methods that use recombinant nucleic acid vectors other than replication incompetent recombinant retroviral particles. For example, other viral vectors can be utilized, such as those derived from adenovirus, adeno-associated virus, or herpes simplex virus-1, as non-limiting examples.

In some embodiments, methods provided herein, and associated uses, reaction mixtures, kits and cell formulations can include transfecting cells with polynucleotides that are not encoded in viral vectors. Such polynucleotides can be referred to as non-viral vectors. In any of the embodiments disclosed herein that utilize non-viral vectors to genetically modify or transfect cells, the non-viral vectors, including for example, plasmids or naked DNA, can be introduced into the cells, such as for example, PBMCs, B cells, T cells and/or NK cells using methods that include electroporation, nucleofection, liposomal formulations, lipids, dendrimers, cationic polymers such as poly(ethylenimine) (PEI) and poly(l-lysine) (PLL), nanoparticles, cell-penetrating peptides, microinjection, and/or non-integrating lentiviral vectors. In some embodiments, the liposomal formulations, lipids, dendrimers, PEI, PLL, nanoparticles, and cell-penetrating peptides can be modified to include lymphocyte-targeting ligands, for example, an anti-CD3 antibody. PEI coupled to anti-CD3 antibodies was shown to efficiently transfect PBMCs with an exogenous nucleic acid (O′Neill et al. Gene Ther. 2001 March; 8(5):362-8). Similarly, nanoparticles made from polyglutamic acid molecules coupled to anti-CD3e f(ab′)2 fragments transfected T lymphocytes (Smith et al. Nat Nanotechnol. 2017 August; 12(8): 813-820). In some embodiments, DNA can be introduced into cells, such as PBMCs, B cells, T cells and/or NK cells in a complex with liposomes and protamine. Other methods for transfecting T cells and/or NK cells ex vivo that can be used in embodiments of methods provided herein, are known in the art (see e.g., Morgan and Boyerinas, Biomedicines. 2016 Apr. 20; 4(2). pii: E9, incorporated by reference herein in its entirety).

In some embodiments of method provided herein, DNA can be integrated into the genome using transposon-based carrier systems by co-transfection, co-nucleofection or co-electroporation of target DNA as plasmid containing the transposon ITR fragments in 5′ and 3′ ends of the gene of interest and transposase carrier system as DNA or mRNA or protein or site specific serine recombinases such as phiC31 that integrates the gene of interest in pseudo attP sites in the human genome, in this instance the DNA vector contains a 34 to 40 bp attB site that is the recognition sequence for the recombinase enzyme (Bhaskar Thyagarajan et al. Site-Specific Genomic Integration in Mammalian Cells Mediated by Phage φC31 Integrase, Mol Cell Biol. 2001 June; 21(12): 3926-3934) and co transfected with the recombinase. For T cells and/or NK cells, transposon-based systems that can be used in certain methods provided herein utilize the Sleeping Beauty DNA carrier system (see e.g., U.S. Pat. No. 6,489,458 and U.S. patent application Ser. No. 15/434,595, incorporated by reference herein in their entireties), the PiggyBac DNA carrier system (see e.g., Manuri et al., Hum Gene Ther. 2010 April; 21(4):427-37, incorporated by reference herein in its entirety), or the ToLCDR2transposon system (see e.g., Tsukahara et al., Gene Ther. 2015 February; 22(2): 209-215, incorporated by reference herein in its entirety) in DNA, mRNA, or protein form. In some embodiments, the transposon and/or transposase of the transposon-based vector systems can be produced as a minicircle DNA vector before introduction into T cells and/or NK cells (see e.g., Hudecek et al., Recent Results Cancer Res. 2016; 209:37-50 and Monjezi et al., Leukemia. 2017 January; 31(1):186-194, incorporated by reference herein in their entireties). However, in some situations, the transposase-based carrier systems are not the preferred method of introducing an exogenous nucleic acid. Thus, in some embodiments, a polynucleotide of any of the aspects or embodiments disclosed herein does not include the transposon ITR fragments. In some embodiments, a modified, genetically modified, and/or transduced cell of any of the aspects or embodiments disclosed herein does not include the transposase carrier system as DNA or mRNA or protein.

The CAR or lymphoproliferative element can also be integrated into the defined and specific sites in the genome using CRISPR or TALEN mediated integration, by adding 50-1000 bp homology arms homologous to the integration 5′ and 3′ of the target site (Jae Seong Lee et al. Scientific Reports 5, Article number: 8572 (2015), Site-specific integration in CHO cells mediated by CRISPR/Cas9 and homology-directed DNA repair pathway). CRISPR or TALEN provide specificity and genomic-targeted cleavage and the construct will be integrated via homology-mediated end joining (Yao X et al. Cell Res. 2017 June; 27(6):801-814. doi: 10.1038/cr.2017.76. Epub 2017 May 19). The CRISPR or TALEN can be co-transfected with target plasmid as DNA, mRNA, or protein.

For any of the methods for modifying, genetically modifying, and/or transducing T and/or NK cells (e.g., in whole blood or in whole blood fractions such as TNCs or PBMCs), or uses that include such methods, or modified cells produced using such methods, and any other method or product-by-process provided herein, a skilled artisan will understand where an exogenous nucleic acid(s) could be introduced into the cells using methods that do not include a replication incompetent recombinant retroviral particle, for example using another type of recombinant vector (e.g. a plasmid associated with a lipid transfection agent).

### Inhibitory RNA Molecules

Embodiments of any of the aspects provided herein can include recombinant retroviral particles whose genomes are constructed to induce expression of one or more, and in illustrative embodiments two or more, inhibitory RNA molecules, such as for example, a miRNA or shRNA, after integration into a host cell, such as a lymphocyte (e.g., a T cell and/or an NK cell). Such inhibitory RNA molecules can be encoded within introns, including for example, an EF1-a intron. This takes advantage of the present teachings of methods to maximize the functional elements that can be included in a packageable retroviral genome to overcome shortcomings of prior teachings and maximize the effectiveness of such recombinant retroviral particles in adoptive T cell therapy.

In some embodiments, the inhibitory RNA molecule includes a 5′ strand and a 3′ strand (in some examples, sense strand and antisense strand) that are partially or fully complementary to one another such that the two strands are capable of forming a 18-25 nucleotide RNA duplex within a cellular environment. The 5′ strand can be 18, 19, 20, 21, 22, 23, 24, or 25 nucleotides in length, and the 3′ strand can be 18, 19, 20, 21, 22, 23, 24, or 25 nucleotides in length. The 5′ strand and the 3′ strand can be the same or different lengths, and the RNA duplex can include one or more mismatches. Alternatively, the RNA duplex has no mismatches. In some illustrative, a vector or genome herein, includes 2 or more, of the inhibitory RNA provided herein.

The inhibitory RNA molecules included in compositions and methods provided herein, in certain illustrative examples, do not exist and/or are not expressed naturally in T cells into whose genome they are inserted. In some embodiments, the inhibitory RNA molecule is a miRNA or an shRNA. In some embodiments, an inhibitory molecule of an embodiment of the present disclosure is a precursor of a miRNA, such as for example, a Pri-miRNA or a Pre-miRNA, or a precursor of an shRNA. In some embodiments, the miRNA or shRNA are artificially derived (i.e., artificial miRNAs or siRNAs). In other embodiments, the inhibitory RNA molecule is a dsRNA (either transcribed or artificially introduced) that is processed into an siRNA or the siRNA itself. In some embodiments, the miRNA or shRNA has a sequence that is not found in nature, or has at least one functional segment that is not found in nature, or has a combination of functional segments that are not found in nature.

In some embodiments, inhibitory RNA molecules are positioned in the first nucleic acid molecule in a series or multiplex arrangement such that multiple miRNA sequences are simultaneously expressed from a single polycistronic miRNA transcript. In some embodiments, the inhibitory RNA molecules can be adjoined to one another either directly or indirectly by non-functional linker sequence(s). The linker sequence in some embodiments, is between 5 and 120 nucleotides in length, and in some embodiments can be between 10 and 40 nucleotides in length, as non-limiting examples. In some embodiments, functional sequences can be expressed from the same transcript as the inhibitory RNA molecules, for example, any of the lymphoproliferative elements provided herein. In some embodiments, the inhibitory RNA molecule is a naturally occurring miRNA such as but not limited to miR-155, miR-30, miR-17-92, miR-122, and miR-21. In some embodiments, an inhibitory RNA molecule includes from 5′ to 3′ orientation: a 5′ microRNA flanking sequence, a 5′ stem, a loop, a 3′ stem that is partially or fully complementary to said 5′ stem, and a 3′ microRNA flanking sequence. In some embodiments, the 5′ stem (also called a 5′ arm herein) is 18, 19, 20, 21, 22, 23, 24 or 25 nucleotides in length. In some embodiments, the 3′ stem (also called a 3′ arm herein) is 18, 19, 20, 21, 22, 23, 24, or 25 nucleotides in length. In some embodiments, the loop is 3 to 40, 10 to 40, 20 to 40, or 20 to 30 nucleotides in length, and in illustrative embodiments the loop can be 18, 19, 20, 21, or 22 nucleotides in length. In some embodiments, one stem is two nucleotides longer than the other stem. The longer stem can be the 5′ or the 3′ stem. The inhibitory RNA molecule can be any of the inhibitory RNA molecules in the Inhibitory RNA Molecules section herein.

In some embodiments, the 5′ microRNA flanking sequence, 3′ microRNA flanking sequence, or both, are derived from a naturally occurring miRNA, such as but not limited to miR-155, miR-30, miR-17-92, miR-122, and miR-21. In certain embodiments, the 5′ microRNA flanking sequence, 3′ microRNA flanking sequence, or both, are derived from a miR-155, such as, e.g., the miR-155 from Mus musculus or Homo sapiens. Inserting a synthetic miRNA stem-loop into a miR-155 framework (i.e., the 5′ microRNA flanking sequence, the 3′ microRNA flanking sequence, and the loop between the miRNA 5′ and 3′ stems) is known to one of ordinary skill in the art (Chung, K. et al. 2006. Nucleic Acids Research. 34(7): e53; U.S. Pat. No. 7,387,896) for example the SIBR and eSIBR sequences. In some embodiments of the present disclosure, miRNAs can be placed in the SIBR or eSIBR miR-155 framework. In illustrative embodiments herein, miRNAs are placed in a miR-155 framework that includes the 5′ microRNA flanking sequence of miR-155 represented by SEQ ID NO:333 or a functional variant thereof, the 3′ microRNA flanking sequence represented by SEQ ID NO:334 (nucleotides 221-265 of the Mus musculus BIC noncoding mRNA) or a functional variant thereof; and a modified miR-155 loop (SEQ ID NO:335) or a functional variant thereof. However, any known microRNA framework that is functional to provide proper processing within a cell of miRNAs inserted therein to form mature miRNA capable of inhibiting expression of a target mRNA to which they bind, is contemplated within the present disclosure.

In some embodiments, where two or more inhibitory RNA molecules (in some examples, 1, 2, 3, 4, 5, 6, 7, 8, 9, or 10 inhibitory RNA molecules) are included, these inhibitory RNA molecules are directed against the same or different RNA targets (such as e.g., mRNAs transcribed from genes of interest).

In some embodiments, the one or more inhibitor RNA molecules are one or more lymphoproliferative elements, accordingly, in any aspect or embodiment provided herein that includes a lymphoproliferative element, unless incompatible therewith, or already stated therein. In illustrative embodiments, miRNAs inserted into the genome of T cells in methods provided herein, are directed at targets such that proliferation of the T cells is induced and/or enhanced and/or apoptosis is suppressed. In some embodiments, the RNA targets are mRNAs transcribed from genes that are miR-155 targets.

In some embodiments, the inhibitory RNA, for example miRNA, targets mRNA encoding ABCG1, Cbl Proto-Oncogene (RNF55) (also known as cCBL and RNF55) (HGNC: 1541, Entrez Gene: 867, OMIM: 165360), T-Cell Receptor T3 Zeta Chain (CD3z) (HGNC: 1677, Entrez Gene: 919, OMIM: 186780), T cell receptor alpha locus (TCRA) (also known as TCRα) (HGNC: 12027, Entrez Gene: 6955, OMIM: 186880), T cell receptor beta locus (TCRB) (also known as TCRβ) (HGNC: 12155, Entrez Gene: 6957, OMIM: 186930), PD1, CTLA4, IFN gamma, T Cell Immunoglobulin Mucin 3 (TIM3) (also known as Hepatitis A Virus Cellular Receptor 2) (HGNC: 18437 Entrez Gene: 84868, OMIM: 606652), Lymphocyte Activating 3 (LAG3) (HGNC: 6476, Entrez Gene: 3902, OMIM: 153337), SMAD2, TNF Receptor Superfamily Member 10b (TNFRSF10B) (HGNC: 11905, Entrez Gene: 8795, OMIM: 603612), Protein Phosphatase 2 Catalytic Subunit Alpha (PPP2CA) (HGNC: 9299, Entrez Gene: 5515, OMIM: 176915), Tumor Necrosis Factor Receptor Superfamily Member 6 (TNFRSF6) (also known as Fas Cell Surface Death Receptor (FAS)) (HGNC: 11920, Entrez Gene: 355, OMIM: 134637), B And T Lymphocyte Associated (BTLA) (HGNC: 21087, Entrez Gene: 151888, OMIM: 607925), T Cell Immunoreceptor With Ig And ITIM Domains (TIGIT) (HGNC: 26838, Entrez Gene: 201633, OMIM: 612859), Adenosine Ata Receptor (ADORA2A or A2AR) (HGNC: 263, Entrez Gene: 135, OMIM: 102776), Aryl Hydrocarbon Receptor (AHR) (HGNC: 348, Entrez Gene: 196, OMIM: 600253), Eomesodermin (EOMES) (HGNC: 3372, Entrez Gene: 8320, OMIM: 604615), SMAD Family Member 3 (SMAD3) (HGNC: 6769, Entrez Gene: 4088, OMIM: 603109), SMAD Family Member 4 (SMAD4) (GNC: 6770, Entrez Gene: 4089, OMIM: 600993), TGFBR2, TRAIL2, PP2A, Protein Phosphatase 2 Regulatory Subunit B delta (PPP2R2D) (HGNC: 23732, Entrez Gene: 55844, OMIM: 613992), Tumor Necrosis Factor Ligand Superfamily Member 6 (TNFSF6) (also known as FASL) (HGNC: 11936, Entrez Gene: 356, OMIM: 134638), Caspase 3 (CASP3) HGNC: 1504, Entrez Gene: 836, OMIM: 600636), SOCS1, Suppressor Of Cytokine Signaling 2 (SOCS2) (HGNC: 19382, Entrez Gene: 8835, OMIM: 605117), Kruppel Like Factor 10 (KLF10) (also known as TGFB-Inducible Early Growth Response Protein 1 (TIEG1)) (HGNC: 11810, Entrez Gene: 7071, OMIM: 601878), JunB Proto-Oncogene, AP-1 Transcription Factor Subunit (JunB) (HGNC: 6205, Entrez Gene: 3726, OMIM: 165161), Chromobox 1 (Cbx) (HGNC: 1551, Entrez Gene: 10951, OMIM: 604511), Cbx3, Tet Methylcytosine Dioxygenase 2 (Tet2) (HGNC: 25941, Entrez Gene: 54790, OMIM: 612839), Hexokinase 2 (HK2) (HGNC: 4923, Entrez Gene: 3099, OMIM: 601125), Src homology region 2 domain-containing phosphatase-1 (SHP1) (HGNC: 9658, Entrez Gene: 5777, OMIM: 176883), Src homology region 2 domain-containing phosphatase-2 (SHP2) (HGNC: 9644, Entrez Gene: 5781, OMIM: 176876), colony stimulating factor 2 (CSF2; GMCSF) (Entrez Gene: 1437). In some embodiments, the inhibitory RNA, for example miRNA, targets the antigen that the ASTR of the CAR binds to.

In some aspects, provided herein is a polynucleotide designed to express a self-driving CAR. Details regarding such replication incompetent recombinant retroviral particles, and composition and method aspects including a self-driving CAR, are disclosed in more detail herein, for example in the Self-Driving CAR Methods and Compositions section and in the Exemplary Embodiments section. In some embodiments, the polynucleotides designed to express a self-driving CAR can include any of the inhibitory RNA molecules disclosed herein. Such polynucleotides can also have inhibitory RNA molecules that target inhibitors of the NFAT pathway, with or without the other inhibitory RNA molecules disclosed herein. In some embodiments, the inhibitory RNA molecules can target CABIN, Homer2, AKAP5, LRRK2, and/or DSCR1/MCIP (knockdown of the RNA molecules encoding these proteins can reduce inhibition of calcineurin or calmodulin); and/or Dyrk1A, CK1, and/or GSK3 (knockdown of the RNA molecules encoding these proteins can prevent phosphorylation, and nuclear export, of NFAT). In some further illustrative embodiments, a vector or genome herein, includes 2 or more, 2-10, 2-8, 2-6, 3-5, 2, 3, 4, 5, 6, 7, or 8 of the inhibitory RNA (e.g., miRNA) identified herein, for example in the paragraph immediately above.

In some embodiments provided herein, the two or more inhibitory RNA molecules can be delivered in a single intron, such as but not limited to EF1-a intron A. Intron sequences that can be used to harbor miRNAs for the present disclosure include any intron that is processed within a T cell. Sequence requirements for introns are known in the art. In some embodiments, such intron processing is operably linked to a riboswitch, such as any riboswitch disclosed herein. Accordingly, in illustrative embodiments provided herein is a combination of an miRNA directed against an endogenous T cell receptor subunit, wherein the expression of the miRNA is regulated by a riboswitch, which can be any of the riboswitches discussed herein.

In some embodiments, inhibitory RNA molecules can be provided on multiple nucleic acid sequences that can be included on the same or a different transcriptional unit. For example, a first nucleic acid sequence can encode one or more inhibitory RNA molecules and be expressed from a first promoter and a second nucleic acid sequence can encode one or more inhibitory RNA molecules and be expressed from a second promoter. In illustrative embodiments, two or more inhibitory RNA molecules are located on a first nucleic acid sequence that is expressed from a single promoter. The promoter used to express such miRNAs, are typically promoters that are inactive in a packaging cell used to express a retroviral particle that will deliver the miRNAs in its genome to a target T cell, but such promoter is active, either constitutively or in an inducible manner, within a T cell. The promoter can be a Pol I, Pol II, or Pol III promoter. In some illustrative embodiments, the promoter is a Pol II promoter.

### TREATMENT METHODS

The present disclosure provides various treatment methods using an engineered T cell receptor or a CAR. An engineered T cell receptor or a CAR of the present disclosure, when present in a T lymphocyte or an NK cell, can mediate cytotoxicity toward a target cell. Such methods typically involve administration of modified lymphocytes, or substantially purified or purified RIR retroviral particles to a subject as provided herein. An engineered T cell receptor or CAR of the present disclosure binds to an antigen present on a target cell, thereby mediating killing of a target cell by a T lymphocyte or an NK cell genetically modified to produce the engineered T cell receptor or CAR. In some embodiments, an engineered T cell receptor or an ASTR of the CAR binds to an antigen present on the surface of a target cell.

The present disclosure provides methods of killing, or inhibiting the growth of, a target cell, the method involving contacting a cytotoxic immune effector cell (e.g., a cytotoxic T cell, or an NK cell) that is genetically modified to produce a subject engineered T cell receptor or CAR, such that the T lymphocyte or NK cell recognizes an antigen present on the surface of a target cell, and mediates killing of the target cell.

The present disclosure provides a method of treating a disease or disorder in an individual having the disease or disorder, the method including: a. introducing an expression vector including a polynucleotide sequence encoding a CAR into peripheral blood cells obtained from the subject to produce a genetically engineered cytotoxic cell; and b. administering the genetically engineered cytotoxic cell to the subject.

Methods provided herein, such as adoptive cell therapies, methods for producing persistent populations of cells, methods for delivering a formulation, etc. as non-limiting examples, are especially adopted for treating cancer. Such cancer can be any type of cancer. For example, such methods can be used for treating a subject who has, or a tumor associated with ovarian cancer, soft tissue sarcoma, peripheral T cell cancer, colorectal cancer, intrahepatic cholangiocarcinoma, glioblastoma, esophageal cancer, cutaneous T cell lymphoma, non-hodgkin lymphoma, urothelial cancer, basal cell carcinoma, epithelioid sarcoma, pancreatic cancer, non-small cell lung carcinoma, hodgkin lymphoma, renal cell carcinoma, mesothelioma, metastatic uveal melanoma, kidney cancer, blood cancer, HER2-expressing cancers, non-melanoma skin cancer, liposarcoma, hepatocellular carcinoma, small lymphocytic lymphoma, prostate cancer, breast cancer, anal cancer, marginal zone lymphoma, cutaneous squamous cell carcinoma, thyroid cancer, medullary thyroid cancer, triple-negative breast cancer, neuroendocrine prostate cancer, bladder cancer, paraganglioma, medulloblastoma, superficial basal cell carcinoma, head and neck squamous cell carcinoma, hematologic malignancies, melanoma, B-cell lymphoma, relapsed/refractory acute myeloid leukemia, angiosarcoma, bone sarcoma, refractory cervical cancer, cholangiocarcinoma, osteosarcoma, biliary tract cancer, castration-resistant prostate cancer, gastroesophageal adenocarcinomas, rhabdomyosarcoma, carcinoma, non-muscle invasive bladder cancer, uveal melanoma, small cell lung cancer, cervical cancer, primary open angle glaucoma, follicular lymphoma, synovial sarcoma, liver cancer, carcinosarcoma, leptomeningeal brain tumors, T-cell lymphoma, lymphoma, small cell lung cancer, mantle cell lymphoma, B-cell malignancies, endometrial cancer, myxoid/round cell liposarcoma, metastatic merkel cell carcinoma, neuroblastoma, chronic lymphocytic leukemia, tenosynovial giant cell tumors, sarcoma, acute myeloid leukemia, skin cancer, nasopharyngeal carcinoma, relapsed/refractory ewing sarcoma, bone cancer, glioma, salivary gland carcinoma, gastric cancer, benign tumor, low-grade serous ovarian cancer, metastatic breast cancer, multiple myeloma, diffuse large B cell lymphoma, relapsed/refractory lymphoma, metastatic colorectal cancer, advanced malignancies, acute lymphoblastic leukemia, mesothelin-expressing solid tumors.

In some embodiments, methods herein can be used to treat tumors that express any one or more of the tumor-associated antigens and/or tumor-specific antigens provided herein, and engineered T cell receptors and CARs can be designed to recognize such targets. As non-limiting examples, such tumor associated or tumor specific antigens include blood tumor antigens provided herein elsewhere in this specification, and in some non-limiting embodiments includes the following antigens, most or all of which are believed to be associated with solid tumors: AXL, CD44v6, CAIX, CEA, CD133, c-Met, EGFR, EGFRvIII, Epcam, EphA2, GD2, GPC3, GUCY2C, HER1, HER2, ICAM-1, IL13Rα2, IL11Rα, Kras, Kras G12D, L1CAM, MAGE, MET, Mesothelin, MUC1, MUC16 ecto, NKG2D, NY-ESO-1, PSCA, ROR-2, WT-1.

In some embodiments, any of the methods provided herein that involve an administering step, can be combined with administration of another cancer therapy, which in certain embodiments, can be a cancer vaccine, for example delivered subcutaneously. In other embodiments and optionally in further combination with cancer vaccine administration, such methods provided herein that include administering genetically modified T cells and/or NK cells into a subject, especially where the subject has, is afflicted with, or is suspected of having cancer, can further include delivering an effective dose of an immune checkpoint inhibitor to the subject. This checkpoint inhibitor delivery can occur before, after, or at the same time as administering the genetically modified T cells and/or NK cells Immune checkpoint inhibitors are known and various compounds are approved and in clinical development. Check point molecules, many of which are the target of checkpoint inhibitor compounds, include, but not limited to an anti-PD1 antibody.

In some embodiments, the administering is for treating cancer in the subject, and wherein a tumor in the subject regresses within 60 days, 45 days, 30 days, or 14 days after said administering. In some embodiments, the tumor is a blood cancer, for example DLBCL, that in illustrative examples expresses any of the blood cancer antigens provided herein. In other embodiments, the tumor is a solid tumor that expresses a solid tumor antigen, which in certain illustrative embodiments is a HER2 positive solid tumor, such as, but not limited to, breast cancer. In some embodiments, the administering is for treating cancer in the subject, and wherein the subject experiences stable disease, at least a partial response, or a complete response, in illustrative embodiments by RECIST1.1 criteria, within 90 days, 75 days, 60 days, 45 days, 30 days, or 14 days after said administering. In some embodiments, the tumor shrinks by at least 10%, 20%, 25%, 30%, 50% or more. In some embodiments, a partial response occurs when the sum of tumor lesions reduces by 30% or more and is confirmed at least 4 weeks after the prior scan without the appearance of new lesions and/or any pathological lymph nodes have a reduction in short axis to less than 10 mm In some embodiments, a complete response occurs when all target and non-target lesions disappear. In some embodiments, the administering is for treating cancer in the subject, and wherein the subject experiences at least a partial response or experiences a complete response within 60 days, 45 days, 30 days, or 14 days after said administering. In some embodiments, the subject is a human afflicted with cancer. In some embodiments, the cell formulation is administered 2, 3, 4, 5, 6, or more times, or in illustrative embodiments only once to the subject before stable disease, or in illustrative embodiments a partial response or a complete response is achieved. In some embodiments, a second formulation is administered to the subject at a second, third, fourth, etc. timepoint between 1 day and 1 month, 2 months, 3 months, 6 months, or 12 months after the administering a first cell formulation, wherein the second formulation can be identical to the first formulation, or can comprises any of the formulations provided herein.

In any of the aspects provided herein that include intramuscular, and in illustrative embodiments subcutaneous administration, of modified lymphocytes (e.g. modified T cells and/or NK cells), in certain embodiments, administration by any route provided herein, is performed on a mammalian subject that has been subjected to a lymphodepletion process, as are known in the art. However, in illustrative embodiments, the administration of the modified T cells and/or NK cells, or RER retroviral particles (RIPs), is performed in a method that does not require lymphodepletion of the subject for successful engraftment in the subject and/or for successful reduction of tumor volume in the subject, or that is performed on a mammalian (e.g. human) subject that has not been subjected to lymphodepletion in the prior days, weeks, or months or ever before such administration (e.g. subcutaneous administration). In certain embodiments, the administration is performed on a mammalian (e.g. human) subject that is not suffering from a low white blood cell count, lymphopenia or lymphocytopenia. In certain embodiments, the subcutaneous administration is performed on a subject having a lymphocyte count in the normal range (i.e., 1,000 and 4,800 lymphocytes in 1 microliter (μL) of blood). In certain embodiments, the subcutaneous administration is performed on a subject having between 1,000 and 5,000, over 300, over 500, over 1,000, over 1,500, or over 2,000 lymphocytes per μL of blood). In certain embodiments, the subcutaneous administration is performed on a mammalian (e.g., human) subject that is lymphoreplete.

### Characterization and Commercial Production Methods

The present disclosure provides various methods and compositions that can be used as research reagents in scientific experimentation and for commercial production. Such scientific experimentation can include methods for characterization of lymphocytes, such as NK cells and in illustrative embodiments, T cells using methods for modifying, for example genetically modifying and/or transducing lymphocytes provided herein. Such methods for example, can be used to study activation of lymphocytes and the detailed molecular mechanisms by which activation makes such cells transducible. Furthermore, provided herein are modified and in illustrative embodiments genetically modified lymphocytes that will have utility for example, as research tools to better understand factors that influence T cell proliferation and survival. Such modified lymphocytes, such as NK cells and in illustrative embodiments T cells, can furthermore be used for commercial production, for example for the production of certain factors, such as growth factors and immunomodulatory agents, that can be harvested and tested or used in the production of commercial products.

The scientific experiments and/or the characterization of lymphocytes can include any of the aspects, embodiments, or subembodiments provided herein useful for analyzing or comparing lymphocytes. In some embodiments, T cells and/or NK cells can be transduced with the replication incompetent recombinant retroviral particles provided herein that include polynucleotides. In some embodiments, transduction of the T cells and/or NK cells can include polynucleotides that include polynucleotides encoding polypeptides of the present disclosure, for example, CARs, lymphoproliferative elements, and/or activation elements. In some embodiments, the polynucleotides can include inhibitory RNA molecules as discussed elsewhere herein. In some embodiments, the lymphoproliferative elements can be chimeric lymphoproliferative elements.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein and all of the possible combinations of the disclosed aspects and embodiments are not listed in this section. Additional embodiments and aspects are provided in other sections herein. Furthermore, it will be understood that embodiments are provided that are specific embodiments for many aspects, as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are discussed more specifically in other sections of this detailed description. Thus, for example any of the embodiments provided herein can be used in any of the reaction mixture, cell formulation, kit, use, cell processing assembly, filter assembly, cell population, modified, genetically modified, and transduced T cell or NK cell, mixtures, cell mixtures, or method aspect provided herein, unless incompatible with, or otherwise stated. Many of the method aspects provided herein, include the following steps that are referred to herein as the “C/F steps”. Such steps include the following: a) contacting cells, such as blood cells comprising NK cells and/or in illustrative embodiments T cells, ex vivo in a reaction mixture comprising an activation element and with recombinant or nucleic acid vectors, in illustrative embodiments a replication incompetent recombinant retroviral particles (“RIPs”), wherein the RIPs comprise a polynucleotide encoding a first polypeptide comprising a transgene, and in illustrative embodiments an antigen, an engineered T cell receptor, or a chimeric antigen receptor (“CAR”) wherein the CAR comprises an antigen-specific targeting region (“ASTR”), a transmembrane domain, and an intracellular activating domain, and/or a lymphoproliferative element (“LE”), wherein said contacting facilitates association of the T cells and/or NK cells with the nucleic acid vectors, in illustrative embodiments the RIPs, and wherein the nucleic acid vectors, in illustrative embodiments the RIPs, modify the T cells and/or NK cells to form a population of modified T cells and/or NK cells; and b) forming a cell formulation by suspending the population of modified T cells and/or NK cells in a delivery solution. These steps can typically include optional incubations and/or a step of washing unbound nucleic acid vector away from the cells in the reaction mixture.

In some illustrative embodiments, referred to herein as the “C/F/A steps,” the following step is performed after the above contacting and forming steps: c) administering the cell formulation to a subject. In some illustrative embodiments, before the contacting step, a step referred to herein as a “collecting step” or a “drawing blood” step, blood comprising lymphocytes, for example T cells and NK cells, typically as well as other whole blood components, such as neutrophils and other component provided herein, is collected from a subject, such as a mammal, for example a domestic animal or in illustrative embodiments, a human, before the contacting step.

It is noteworthy that in certain illustrative embodiments, the reaction mixture includes unfractionated whole blood or includes all or many cell types found in whole blood, including total nucleated cells (TNCs). It is noteworthy that in certain embodiments, the recombinant vector comprises a self-driving CAR, which encodes both a CAR and a lymphoproliferative element. Provided later in this Exemplary Embodiments section are exemplary ranges and lists that can be used for any of the aspects provided immediately below or otherwise herein, unless incompatible with or otherwise indicated, as will be recognized by a skilled artisan.

In one aspect, provide herein is a method for administering modified T cells and/or NK cells to a subject, comprising the C/F/A steps. In another aspect, provided herein is a method for administering a cell formulation to a subject, comprising the C/F/A steps. In another embodiment, provided herein is a method of generating a persisting population of genetically modified cells in a subject, comprising the C/F/A steps, wherein the persisting population of genetically modified lymphocytes persists in the subject for at least 7, 14, 21, or 28 days or 1, 2, 3, 4, 5, 6, 7, 8, 9, 10, 11, or 12 months or 1, 2, 3, 4, or 5 years after administration. In another aspect, provided herein is a method for subcutaneous or intramuscular delivery of modified T cells and/or NK cells to a subject, comprising the C/F/A steps. In another aspect, provided herein is a method of performing cell therapy, comprising the C/F/A steps. In another aspect, provided herein is a method of expanding a population of genetically modified lymphocytes in a subject, comprising the C/F/A steps, wherein the administered modified lymphocytes produce a population of progeny genetically modified lymphocytes in the subject.

In another aspect, provided herein is a method of performing CAR-T therapy on a subject afflicted with cancer, comprising the C/F/A steps. In another aspect, provided herein is a method of treating a subject having a disease, disorder, or condition associated with an elevated expression of an antigen, in illustrative embodiments cancer, comprising the C/F/A steps. In another aspect, provided herein is a method of treating a subject with a cancer, the C/F/A steps. In another aspect, provided herein is a method of providing an anti-tumor immunity in a subject, comprising CFA, wherein the anti-tumor immunity response is an active or passive immune response to an antigen expressed by the tumor, comprising the C/F/A steps. In another aspect, provided herein is a method of stabilizing or reducing, tumor burden in a subject, comprising the C/F/A steps. In another aspect, provided herein is a method for providing an anti-tumor immunity in a subject, comprising the C/F/A steps, wherein the anti-tumor immunity response is an active or passive immune response to an antigen expressed by the tumor. In another aspect, provided herein is a method for stimulating a T cell-mediated immune response to a target cell population or tissue in a subject, comprising the C/F/A steps.

Provided herein in one aspect is a method for administering, injecting or delivering modified lymphocytes (e.g. NK cells and/or T cells) to a subject, comprising administering a cell formulation comprising the modified lymphocytes (e.g. T cells and/or NK cells) to the subject subcutaneously, wherein the modified T cells and/or NK cells are either or both, [i] genetically modified with a polynucleotide comprising one or more transcriptional units, wherein each of the one or more transcriptional units is operatively linked to a promoter active in T cells and/or NK cells, or [ii] associated with a RIP comprising the polynucleotide, wherein the one or more transcriptional units encode a first polypeptide comprising a CAR, and wherein at least one of neutrophils, B cells, monocytes, basophils, and eosinophils are administered subcutaneously in the cell formulation along with the modified T cells and/or NK cells.

Provided herein in one aspect is a method for delivering modified lymphocytes (e.g., T cells and/or NK cells) to a subject, or the cell formulation included in the method, comprising administering a cell formulation comprising the modified lymphocytes (e.g. T cells and/or NK cells) to the subject subcutaneously, wherein the modified lymphocytes (e.g. T cells and/or NK cells) are either or both, associated (modified) with a RIP comprising a polynucleotide comprising one or more transcriptional units operatively linked to a promoter active in T cells and/or NK cells, or genetically modified with the polynucleotide, wherein the one or more transcriptional units encode a first polypeptide comprising a CAR, and wherein
i) the polynucleotide is extrachromosomal in at least 10%, 25%, 50%, 75%, 80%, 90% or 95% of the modified lymphocytes;
ii) at least 25%, 50%, 75%, 80%, 90% or 95% of the modified T cells and/or NK cells in the cell formulation do not express one or more of the CAR or a transposase;
iii) at least 25% 50%, 75%, 80%, 90% or 95% of the modified T cells and/or NK cells in the cell formulation comprise a recombinant viral reverse transcriptase or a recombinant viral integrase;
iv) at least 25%, 50%, 75%, 80%, 90% or 95% of the modified T cells and/or NK cells in the cell formulation do not have the polynucleotide stably integrated into their genomes;
v) between 1% and 20%, or optionally between 5% and 15% of T cells and/or NK cells in the cell formulation are genetically modified;
vi) at least 25%, 50%, 75%, 80%, 90% or 95% of the modified T cells and/or modified NK cells in the cell formulation are viable; and/or
vii) at least 10%, 20%, 30%, 40%, 50% of the modified lymphocytes comprise a viral pseudotyping element and/or a T cell activating antibody on their surface.

As indicated, the formulations provided in the method immediately above, itself represents another aspect provided herein. Such formulation aspects can provide any of the cell aggregate embodiments provided herein, the Small Volume Elements provided herein, the Dimmed T Cell Characteristics and the Dimmed NK Cell Characteristics provided herein. In certain embodiments, the cells were ex vivo for less than 24 hours, 12, hours, 8 hours, 6 hours, 4 hours, 2 hours or 1 hour, prior to being added to form the formulation.

In some embodiments, the modified lymphocytes introduced into the subject by intradermal, intratumoral, intraperitoneal, subcutaneous or intramuscular administration, delivery, or injection can be allogeneic lymphocytes. In such embodiments, the lymphocytes are from a different person, and the lymphocytes from the subject are not modified. In some embodiments, no blood is collected from the subject to harvest lymphocytes.

In any of the aspects immediately above, the lymphocytes can be considered modified lymphocytes because either or both, they are associated with a recombinant nucleic acid vector, such as a RIP, comprising a polynucleotide comprising one or more transcriptional units, wherein each transcriptional unit is operatively linked to a promoter active in T cells and/or NK cells, or because they are genetically modified with the polynucleotide, including being transduced with the polynucleotide.

Provided herein in one aspect is a method for delivering, injecting, or administrating modified T cells and/or NK cells to a subject, comprising:
a) optionally collecting blood comprising lymphocytes from the subject;
b) contacting blood cells comprising the T cells and/or NK cells ex vivo in a reaction mixture comprising a T cell and/or NK cell activation element, with the RIPs, wherein the RIPs comprise
   i) a binding polypeptide and a fusogenic polypeptide on the surface of the retroviral particles, wherein the binding peptide is capable of binding to a T cell and/or NK cell, and wherein the fusogenic polypeptide is capable of mediating fusion of a retroviral particle membrane with a T cell and/or an NK cell membrane; and
   ii) a polynucleotide comprising one or more transcriptional units, wherein each of the one or more transcriptional units is operatively linked to a promoter active in T cells and/or NK cells, wherein the one or more transcriptional units encode a first polypeptide comprising a CAR,
   wherein said contacting facilitates association of the T cells and/or NK cells with the RIPs, and wherein the recombinant retroviral particles modify the T cells and/or NK cells; and;
c) administering a solution comprising the modified T cells and/or NK cells to the subject intramuscularly, or in illustrative embodiments subcutaneously, wherein
   i) the reaction mixture comprises at least 25% unfractionated whole blood by volume,
   ii) the reaction mixture comprises neutrophils,
   iii) the modified T cells and/or NK cells are administered subcutaneously in a cell formulation along with one or more of B cells, neutrophils, monocytes, basophils, and eosinophils, and/or
   iv) no more than 14 hours pass between the time blood is collected from the subject and the time the modified T cells and/or NK cells are administered (or readministered/reintroduced) into the subject. In illustrative embodiments, such recombinant nucleic acid vectors are replication incompetent retroviral particles comprising a pseudotyping element on their surface.

In some embodiments for any methods provided herein that include an administering step including, but not limited to, the method immediately above, the method further comprises after the modifying but before the administering, formulating the modified lymphocytes in a dilution solution to form a cell formulation comprising the modified lymphocytes, and wherein the solution administered to the subject is the cell formulation.

Provided herein in one aspect is a method for administering modified lymphocytes to a subject, comprising:
a) collecting blood comprising lymphocytes from the subject;
b) modifying the lymphocytes by contacting the lymphocytes ex vivo in a reaction mixture comprising the blood or a fraction thereof, with recombinant nucleic acid vectors, wherein said contacting is performed without any incubation, or by incubating the reaction mixture for between 1 minute and 12 hours, and wherein said contacting facilitates association of the lymphocytes with the recombinant nucleic acid vector, thereby modifying the lymphocytes; and
c) administering a solution comprising the modified lymphocytes to the subject subcutaneously or intramuscularly, wherein the modified lymphocytes are modified by either or both, being associated with recombinant nucleic acid vectors comprising a polynucleotide comprising one or more transcriptional units operatively linked to a promoter active in T cells and/or NK cells, or by being genetically modified with the polynucleotide, wherein the one or more transcriptional units encode a first polypeptide comprising a CAR. In illustrative embodiments, such recombinant nucleic acid vectors are replication incompetent retroviral particles comprising a fusogenic polypeptide, a binding polypeptide (e.g., pseudotyping element), and optionally an activating element on their surface

In another aspect, provided herein is a method of delivering modified T cells and/or NK cells to a subject, wherein the method comprises, delivering a cell formulation comprising the modified T cells and/or NK cells to the subject subcutaneously, wherein the modified T cells and/or NK cells are genetically modified with a polynucleotide comprising one or more transcriptional units, wherein each of the one or more transcriptional units is operatively linked to a promoter active in T cells and/or NK cells, and wherein the one or more transcriptional units encodes a first polypeptide comprising a CAR and a second polypeptide comprising an LE that comprises an intracellular signaling domain from a cytokine receptor.

Provided herein in one aspect is a cell formulation, and a use of recombinant nucleic acid vectors, in illustrative embodiments replication incompetent retroviral particles, to make, or in the manufacture of, a cell formulation, comprising modified lymphocytes (e.g. T cells and/or NK cells), and in illustrative embodiments tumor infiltrating lymphocytes or genetically modified lymphocytes, for administering the modified lymphocytes to a subject subcutaneously or intramuscularly, wherein the recombinant nucleic acid vectors comprise a polynucleotide comprising one or more transcriptional units, wherein each of the transcriptional units is operatively linked to a promoter active in T cells and/or NK cells, wherein the one or more transcriptional units encode a first polypeptide comprising a CAR, and wherein the cell formulation is effective for, adapted for, and/or capable of subcutaneous or intramuscular administration. The cell formulation can further comprise any of the cell formulation components provided herein.

Provided herein in one aspect is a cell formulation, comprising modified lymphocytes in a delivery solution, wherein the modified lymphocytes have one or more gene vectors, for example replication incompetent recombinant retroviral particles (RIPs) associated with their surfaces, and wherein the modified lymphocytes comprise T cells and/or NK cells, wherein the T cells comprise CD4+ cells and CD8+ cells, and wherein the NK cells comprise CD56+ cells,

wherein the gene vectors or replication incompetent recombinant retroviral particles comprise a polynucleotide encoding a transgene, in illustrative embodiments an antigen, an engineered T cell receptor, a chimeric antigen receptor (CAR),

wherein the gene vectors or replication incompetent recombinant retroviral particles comprise a polypeptide capable of binding to a surface polypeptide, in illustrative embodiments T cell receptor complex polypeptide, or in illustrative embodiments CD3, associated with the surface of the gene vectors or replication incompetent recombinant retroviral particles,

wherein at least 50% of the T cells and/or NK cells in the cell formulation are surface negative for the surface polypeptide or are surface negative for the T cell receptor complex polypeptide, or are surface CD3−, and

wherein at least 5% of the modified lymphocytes are in cell aggregates. In some embodiments, at least 10% of the CD4+ cells and/or CD8+ cells and/or CD56+ cells are in cell aggregates. In some embodiments, at least 50% of the CD4+ cells and/or CD8+ cells in the cell formulation are surface CD3−. In some embodiments, at least 90% of the CD4+ cells and/or CD8+ cells in the cell formulation are surface CD3−. In some embodiments, at the time of the forming and/or the administering between 50% and 99% of the CD4+ cells and/or CD8+ cells in the cell formulation are surface CD3−. In some embodiments, the cell aggregates comprise 5 to 500 modified lymphocytes. In some embodiments, the cell aggregates are capable of being retained by a course filter having a pore diameter of at least 40 μm. In some embodiments, the cell aggregates are greater than 40 μm in diameter. In some embodiments, the cell formulation comprises between 3×10⁴ and 3×10⁹ modified lymphocytes. In some embodiments, the cell formulation has a volume between 0.5 ml and 20 ml and is contained within a syringe. In some embodiments, the cell formulation has a volume between 1 ml and 10 ml and is contained within a syringe. In some embodiments, the cell formulation has a volume between 2 ml and 7 ml and is contained within a syringe. In some embodiments, at least 10% of the modified lymphocytes are in cell aggregates, and wherein the cell formulation is in a syringe and has a volume of between 2 ml and 7 ml. In some embodiments, the cell formulation further comprises neutrophils. In some embodiments, the cell formulation comprises all types of nucleated blood cells and optionally such cells are in a ratio present in blood. In some embodiments, wherein the formulation comprises all types of peripheral blood mononuclear cells and optionally such cells are in a ratio present in peripheral blood.

In another aspect, provided herein is a cell formulation, comprising modified cells, and in illustrative embodiments modified T cells and/or NK cells, in a delivery solution, wherein the modified cells have RIPs associated with their surfaces,

wherein the RIPs comprise a polynucleotide encoding a transgene, and in illustrative embodiments an antigen, an engineered T cell receptor, or a CAR,

wherein the RIPs comprise a polypeptide capable of binding to a TCR complex polypeptide, and in illustrative embodiments CD3, associated with the surface of the RIPs,

wherein at least some of the cells are dimmed as provided herein, for example have one or more characteristics from the Dimmed T Cell Characteristics and/or Dimmed NK Cell Characteristics; and wherein at least some of the cells are in cell aggregates as provided herein.

In some aspects, provided herein is a cell formulation, comprising modified cells, and in illustrative embodiments modified T cells and/or NK cells, in a delivery solution, wherein the modified cells have RIPs associated with their surfaces,

wherein the RIPs comprise a polynucleotide encoding a transgene and optionally encoding a lymphoproliferative element,

wherein the RIPs comprise a polypeptide capable of binding to a TCR complex polypeptide, and in illustrative embodiments CD3, associated with the surface of the RIPs, and

wherein:
i) at least some of the cells are dimmed as provided herein, for example have one or more characteristics from the Dimmed T Cell Characteristics and/or Dimmed NK Cell Characteristics;
ii) at least some of the cells are in cell aggregates as provided herein; and/or
iii) the volume of the cell formulation, or a volume of blood collected or a volume of the reaction mixture, is in the Small Volume Elements.

In another aspect, provided herein is a cell population, comprising subcutaneous T cells and/or NK cells, wherein at least 10%, 20%, 30%, 40%, 50%, 75%, of the T cells and/or NK cells are modified cells having RIPs associated with their surfaces,

wherein the RIPs comprise a polynucleotide encoding a transgene, and in illustrative embodiments an antigen, an engineered T cell receptor, or a chimeric antigen receptor (CAR),

wherein the RIPs comprise a polypeptide that binds a TCR complex polypeptide, and in illustrative embodiments CD3, associated with the surface of the retroviral particles, and

wherein at least some of the cells are dimmed as provided herein, for example have one or more characteristics from the Dimmed T Cell Characteristics and/or Dimmed NK Cell Characteristics.

In one aspect, provided herein is a population of genetically modified lymphocytes, comprising: at least 10, 100, 1×10³, 1×10⁴, 1×10⁵, 1×10⁶, 1×10⁷, 1×10⁸,1×10⁹, 1×10¹⁰, or 1×10¹¹ genetically modified lymphocytes expressing a transgene, in illustrative embodiments and antigen, an engineered T cell receptor, or a chimeric antigen receptor (CAR), wherein at least some of the genetically modified lymphocytes are localized subcutaneously in a subject, and wherein the genetically modified lymphocytes comprise T cells and/or NK cells. In some embodiments, the cell population further comprises other white blood cells that do not express the CAR. In some embodiments, the cell population comprises one or more aggregates of at least 10, 20, 30, 40, 50, 100, or 1,000 cells each.

In another aspect, provided herein is a subcutaneous lymphoid structure, which can be considered a tertiary lymphoid structure, that comprises at least some of the modified lymphocytes of a population of genetically modified lymphocytes, of the cell population aspect immediately above, or any cell population provided herein. In some embodiments, some of the genetically modified lymphocytes expressing the CAR are located in lymphatic vasculature. In some embodiments, the other white blood cells comprise B cells, macrophages, dendritic cells, T cells and/or NK cells. In some embodiments, some of the modified lymphocytes of the population are in lymphatic vasculature localized near, in certain embodiments within 25, 50, 75, 100, 125, 150, 200, 250, 500, or 1,000 μm from, the subcutaneous lymphoid structure. In some embodiments, the subcutaneous lymphoid structure or the population of genetically modified lymphocytes further comprises actively dividing lymphocytes that are native to the subject and do not express the CAR. In some embodiments, the genetically modified lymphocytes express a lymphoproliferative element. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node. some embodiments, the subcutaneous lymphoid structure is an artificial lymph node. In some embodiments, the population of genetically modified lymphocytes is in an artificial lymph node. In some embodiments, the subcutaneous lymphoid structure is an artificial lymph node. In some embodiments, the population of genetically modified lymphocytes is in an artificial lymph node.

In some embodiments, the subcutaneous cell populations and/or lymphoid structures herein are resolvable, transient, and/or dynamic structures of subcutaneous modified lymphocytes provided herein. Accordingly, such populations and structures in some embodiments, occur and/or increase in size and/or number of modified cells therein, at 1, 2, 4, 5, 7, or 14 days post subcutaneous-administration according to any of the methods herein, but then can decrease in size and/or number of modified cells therein in the subcutaneous region of the subject, at later time points, for example at 21 days, 28 days, or later time points. As such, provided herein are resolvable lymphoid structures of cell populations comprising any of the modified lymphocytes provided herein.

In some embodiments, the T cells comprises CD4+ and CD8+ cells, and wherein at least 50% of the genetically modified lymphocytes that are CD4+ and/or CD8+are CD3−;

In some embodiments of any of the population of genetically modified lymphocytes aspects or embodiments herein,
i) the population of genetically modified lymphocytes comprises a persisting population of genetically modified lymphocytes expressing the transgene, the engineered T cell receptor, or the chimeric antigen receptor (CAR), that persists in the subject for at least 7, 14, 21, or 28 days or 1, 2, 3, 4, 5, 6, 7, 8, 9, 10, 11, or 12 months or 1, 2, 3, 4, or 5 years after administration.
ii) the genetically modified lymphocytes produce a population of progeny lymphocytes, wherein the population of progeny lymphocytes comprises at least 1×10⁵, 1×10⁶, 1×10⁷, 1×10⁸, 1×10⁹, 1×10¹⁰, or 1×10¹¹, or between 1×10⁶ and 1×10¹⁰, or between 1×10⁸ and 1×10¹² cells;
iii) the population of genetically modified lymphocytes comprises at least 100 genetically modified lymphocytes localized subcutaneously and the subcutaneous region contains no artificial matrix components; and/or
iv) the at least 10 genetically modified lymphocytes of the population remain localized subcutaneously for at least 7, 14, 21, or 28 days, and in illustrative embodiments, the subcutaneous region contains no artificial matrix components.

In some embodiments, at least 1×10⁵, 1×10⁶, 1×10⁷, 1×10⁸, 1×10⁹, 1×10¹⁰, or 1×10¹¹, or between 1×10⁶ and 1×10¹⁰, or between 1×10⁸ and 1×10¹² cells of the genetically modified lymphocytes are located subcutaneously. In some embodiments, at least 1×10⁵, 1×10⁶, 1×10⁷, 1×10⁸, 1×10⁹, 1×10¹⁰, ort 1×10¹¹, or between 1×10⁶ and 1×10¹⁰, or between 1×10⁸ and 1×10¹² cells are not in the subcutaneous region, and in illustrative embodiments are circulating in the blood and/or at the site of a tumor in the subject.

In one aspect, provided herein is a subcutaneous lymphoid structure, comprising: cell aggregates, wherein the cell aggregates comprise:
a) at least 10, 100, 1×10³, 1×10⁴, 1×10⁵, 1×10⁶, 1×10⁷, 1×10⁸,1×10⁹, 1×10¹⁰, or 1×10¹¹ genetically modified lymphocytes expressing a transgene, in illustrative embodiments and antigen, an engineered T cell receptor, or a chimeric antigen receptor (CAR), wherein the cell aggregates are localized subcutaneously in a subject, and wherein the genetically modified lymphocytes comprise T cells and/or NK cells; and
b) other white blood cells that do not express the CAR, wherein at least 10% of the cells in the cell aggregates are other white blood cells.

In another aspect, provided herein is a method for preparing a cell formulation, comprising
a) contacting blood cells comprising T cells and/or NK cells ex vivo in a reaction mixture comprising a T cell and/or NK cell activation element and replication incompetent recombinant retroviral particles, wherein the replication incompetent recombinant retroviral particles (RIPs) comprise a polynucleotide encoding a first polypeptide comprising a transgene, and in illustrative embodiments an antigen, an engineered T cell receptor, or a chimeric antigen receptor (CAR), and optionally in illustrative embodiments a second polynucleotide encoding a LE;
wherein said contacting facilitates association of the T cells and/or NK cells with the RIPs, and wherein the RIPs modify the T cells and/or NK cells to form a population of modified T cells and/or NK cells; and
b) forming a cell formulation by suspending the population of modified T cells and/or NK cells in a delivery solution, wherein:
   i) at least some of the cells in the cell formulation are dimmed as provided herein, for example the cell formulation has one or more characteristics from the Dimmed T Cell Characteristics and/or Dimmed NK Cell Characteristics;
   ii) at least some of the cells in the cell formulation are in cell aggregates as provided herein;
   iii) the volume of the cell formulation, the volume of the reaction mixture, or a volume of blood collected comprising the blood cells is in the Small Volume Elements; and/or
   iv) upon administration to a subject, in illustrative embodiments, subcutaneous administration, the population of modified T cells and/or NK cells is capable of persisting within a subject for at least 1, 2, 3, 4, 5, 6, 7, 14, 17, 21, or 28 days or 1, 2, or 1, 2, 3, 4, 5, 6, 7, 8, 9, 10, 11, or 12 months or 1, 2, 3, 4, or 5 years after administration.

In some embodiments of any of the aspects provided herein, including but not limited to those provided hereinabove in this Exemplary Embodiments, at least some of the cells in a cell formulation or population are dimmed as provided herein, for example the cell formulation or population has one or more characteristics from the Dimmed T Cell Characteristics and/or Dimmed NK Cell Characteristics.

In some embodiments of any of the aspects provided herein, including but not limited to those provided hereinabove in this Exemplary Embodiments, cells or a population of cells can form or be capable of forming a persisting population of cells that persists or is capable of persisting within a subject for at least 1, 2, 3, 4, 5, 6, 7, 14, 17, 21, or 28 days or 1, 2, or 1, 2, 3, 4, 5, 6, 7, 8, 9, 10, 11, or 12 months or 1, 2, 3, 4, or 5 years after administration.

In some embodiments of any of the aspects provided herein, including but not limited to those provided hereinabove in this Exemplary Embodiments, at least some of the cells in a cell formulation or population are in cell aggregates as provided herein.

In some embodiments of any of the aspects provided herein, including but not limited to those provided hereinabove in this Exemplary Embodiments, a volume of the cell formulation, a volume of blood collected, or a volume of the reaction mixture is in the Small Volume Elements.

In another aspect, provided herein is a persisting population of cells, comprising modified T cells and/or NK cells, wherein the modified cells express
i) an engineered T cell receptor or a CAR, and
ii) a lymphoproliferative element, and
wherein the modified cells of the persisting population and/or parent cells thereof, persisted subcutaneously for at least 28 days in the mammal

In another aspect, provided herein is a persisting population of cells, comprising modified T cells, wherein the modified cells of the persisting population express an engineered T cell receptor or CAR, and a lymphoproliferative element. wherein the modified cells of the persisting population and/or parent cells thereof, are derived from parent cells capable of, or are adapted for forming a persistent subcutaneous cell population capable of persisting for 28 days in the mammal

In another aspect, provided herein is a persisting population of cells, comprising modified T cells, wherein the persisting population is a subcutaneous cell population and the modified cells of the cell population express
i) an engineered T cell receptor or a CAR, and
ii) a LE, wherein the cell population comprises at least 1×10⁶, 1×10⁷, 1×10⁸, or, 1×10⁹, or between at least 1×10⁵ and 1×10⁷, 1×10⁸ or 1×10⁹ modified cells.

In another aspect, provided herein is a subcutaneous cell population, comprising cell aggregates of genetically modified T cells and/or NK cells expressing an engineered T cell receptor or CAR, wherein the subcutaneous cell population is formed from cells administered at a site of administration, and wherein 10%, 20%, 30%, 40%, 50%, 60%, 70%, 80%, 90%, or 95% of the cells in the subcutaneous cell population remain localized within 1, 2, 3, 4, or 5 cm of the site of administration.

Provided herein in a set of related aspects is a method for administering a cell formulation to a subject, or a use of gene vectors or replication incompetent recombinant retroviral particles in the manufacture of a kit for administering a cell formulation to a subject, wherein the method, or the use of the kit comprises:
a) contacting blood cells comprising lymphocytes ex vivo in a reaction mixture comprising a T cell and/or NK cell activation element and one or more gene vectors or replication incompetent recombinant retroviral particles (RIPs), wherein the gene vectors or RIPs comprise a polynucleotide encoding a first polypeptide comprising a transgene, and in illustrative embodiments an antigen, an engineered T cell receptor, a chimeric antigen receptor (CAR),
wherein the lymphocytes comprise T cells and/or NK cells, wherein the T cells comprise CD4+ cells and CD8+ cells, and wherein the NK cells comprise CD56 lymphocytes, and
wherein said contacting facilitates association of the lymphocytes with the RIPs, and wherein the RIPs modify the T cells and/or NK cells to form a population of modified lymphocytes comprising modified T cells and/or NK cells;
b) forming a cell formulation by suspending the population of modified lymphocytes in a delivery solution; and
c) administering the cell formulation to a subject through subcutaneous, intramuscular, intraperitoneal, intratumoral, or intravenous administration, wherein at least 5% of the modified lymphocytes are in cell aggregates, wherein at the time of the forming and/or the administering at least 50% of the T cells, CD4+ cells and/or CD8+ cells and/or CD56+ cells in the cell formulation are surface negative for a surface polypeptide or are surface negative for a T cell receptor complex polypeptide, or surface CD3−. In some embodiments, at least 10% of the CD4+ cells and/or CD8+ cells and/or CD56+ cells are in cell aggregates. In some embodiments, at least 50% of the CD4+ cells and/or CD8+ cells in the cell formulation are surface CD3−. In some embodiments, at least 90% of the CD4+ cells and/or CD8+ cells in the cell formulation are surface CD3−. In some embodiments, at the time of the forming and/or the administering between 50% and 99% of the CD4+ cells and/or CD8+ cells in the cell formulation are surface CD3−. In some embodiments, the cell aggregates comprise 5 to 500 modified lymphocytes. In some embodiments, the cell aggregates are capable of being retained by a course filter having a pore diameter of at least 40 μm. In some embodiments, the cell aggregates are greater than 40 μm in diameter. In some embodiments, the cell formulation comprises between 3×10⁴ and 3×10⁹ modified lymphocytes. In some embodiments, the cell formulation has a volume between 0.5 ml and 20 ml and is contained within a syringe. In some embodiments, the cell formulation has a volume between 1 ml and 10 ml and is contained within a syringe. In some embodiments, the cell formulation has a volume between 2 ml and 7 ml and is contained within a syringe. In some embodiments, at least 10% of the modified lymphocytes are in cell aggregates, and wherein the cell formulation is in a syringe and has a volume of between 2 ml and 7 ml. In some embodiments, the cell formulation further comprises neutrophils. In some embodiments, the cell formulation comprises all types of nucleated blood cells and optionally such cells are in a ratio present in blood. In some embodiments, wherein the formulation comprises all types of peripheral blood mononuclear cells and optionally such cells are in a ratio present in peripheral blood. In some embodiments, the use further comprises collecting blood comprising the lymphocytes contacted in the reaction mixture from the subject before the contacting, and in some embodiments, between 5 ml and 50 ml or between 5 ml and 30 ml of blood is collected from the subject. In some embodiments, the reaction mixture has a volume of between 5 ml and 30 ml, or
c) administering the cell formulation to a subject through subcutaneous, intramuscular, intraperitoneal, intratumoral, or intravenous administration, wherein at least 5% of the modified lymphocytes are in cell aggregates, wherein at the time of the forming and/or the administering at least 50% of the CD4+ cells and/or CD8+ cells and/or CD56+ cells in the cell formulation are surface negative for a surface polypeptide or are surface negative for the T cell receptor complex polypeptide, or surface CD3−. In some embodiments, at least 10% of the CD4+ cells and/or CD8+ cells and/or CD56+ cells are in cell aggregates. In some embodiments, at least 50% of the CD4+ cells and/or CD8+ cells in the cell formulation are surface CD3−. In some embodiments, at least 90% of the CD4+ cells and/or CD8+ cells in the cell formulation are surface CD3−. In some embodiments, at the time of the forming and/or the administering between 50% and 99% of the CD4+ cells and/or CD8+ cells in the cell formulation are surface CD3−. In some embodiments, the cell aggregates comprise 5 to 500 modified lymphocytes. In some embodiments, the cell aggregates are capable of being retained by a course filter having a pore diameter of at least 40 μm. In some embodiments, the cell aggregates are greater than 40 μm in diameter. In some embodiments, the cell formulation comprises between 3×10⁴ and 3×10⁹ modified lymphocytes. In some embodiments, the cell formulation has a volume between 0.5 ml and 20 ml and is contained within a syringe. In some embodiments, the cell formulation has a volume between 1 ml and 10 ml and is contained within a syringe. In some embodiments, the cell formulation has a volume between 2 ml and 7 ml and is contained within a syringe. In some embodiments, at least 10% of the modified lymphocytes are in cell aggregates, and wherein the cell formulation is in a syringe and has a volume of between 2 ml and 7 ml. In some embodiments, the cell formulation further comprises neutrophils. In some embodiments, the cell formulation comprises all types of nucleated blood cells and optionally such cells are in a ratio present in blood. In some embodiments, wherein the formulation comprises all types of peripheral blood mononuclear cells and optionally such cells are in a ratio present in peripheral blood. In some embodiments, the use further comprises collecting blood comprising the lymphocytes contacted in the reaction mixture from the subject before the contacting, and in some embodiments, between 5 ml and 50 ml or between 5 ml and 30 ml of blood is collected from the subject, or
c) administering the cell formulation to a subject through subcutaneous, intramuscular, intraperitoneal, intratumoral, or intravenous administration,
wherein at the time of the forming and/or the administering at least 5% of the modified T cells are in cell aggregates, wherein at the time of the forming and/or the administering at least 50% of the modified CD4+ cells and/or CD8+ cells in the cell formulation are surface CD3−, wherein the modified T cells in the cell formulation are capable of producing a persisting population of genetically modified lymphocytes expressing the first polypeptide comprising the CAR, wherein the persisting population of genetically modified lymphocytes is capable of persisting in the subject for at least 7 days after administration, and/or wherein the cell formulation has a volume between 0.5 ml and 10 ml contained within a syringe. In some embodiments, at least 10% of the CD4+ cells and/or CD8+ cells and/or CD56+ cells are in cell aggregates. In some embodiments, at least 50% of the CD4+ cells and/or CD8+ cells in the cell formulation are surface CD3−. In some embodiments, at least 90% of the CD4+ cells and/or CD8+ cells in the cell formulation are surface CD3−. In some embodiments, at the time of the forming and/or the administering between 50% and 99% of the CD4+ cells and/or CD8+ cells in the cell formulation are surface CD3−. In some embodiments, the cell aggregates comprise 5 to 500 modified lymphocytes. In some embodiments, the cell aggregates are capable of being retained by a course filter having a pore diameter of at least 40 μm. In some embodiments, the cell aggregates are greater than 40 μm in diameter. In some embodiments, the cell formulation comprises between 3×10⁴ and 3×10⁹ modified lymphocytes. In some embodiments, the cell formulation has a volume between 0.5 ml and 20 ml and is contained within a syringe. In some embodiments, the cell formulation has a volume between 1 ml and 10 ml and is contained within a syringe. In some embodiments, the cell formulation has a volume between 2 ml and 7 ml and is contained within a syringe. In some embodiments, at least 10% of the modified lymphocytes are in cell aggregates, and wherein the cell formulation is in a syringe and has a volume of between 2 ml and 7 ml. In some embodiments, the cell formulation further comprises neutrophils. In some embodiments, the cell formulation comprises all types of nucleated blood cells and optionally such cells are in a ratio present in blood. In some embodiments, wherein the formulation comprises all types of peripheral blood mononuclear cells and optionally such cells are in a ratio present in peripheral blood. In some embodiments, the use further comprises collecting blood comprising the lymphocytes contacted in the reaction mixture from the subject before the contacting, and in some embodiments, between 5 ml and 50 ml or between 5 ml and 30 ml of blood is collected from the subject. In some embodiments, the reaction mixture has a volume of between 5 ml and 30 ml. In some embodiments, administered modified lymphocytes in the cell formulation produce a persisting population of genetically modified lymphocytes expressing the first polypeptide comprising the transgene, in illustrative embodiments the antigen, the engineered T cell receptor, or the CAR, wherein the persisting population of genetically modified lymphocytes persists or is capable of persisting in the subject for at least 7, 14, 21, or 28 days or 1, 2, 3, 4, 5, 6, 7, 8, 9, 10, 11, or 12 months or 1, 2, 3, 4, or 5 years after administration, and wherein the persisting population of genetically modified lymphocytes comprises genetically modified T cells and/or NK cells, and in illustrative embodiments wherein the persisting population of genetically modified lymphocytes persists in the subject for at least 28 days after administration, and wherein at least 50%, 60%, 70%, 80%, 90% or 95% of the genetically modified lymphocytes expressing the first polypeptide comprising the transgene, the antigen, the engineered T cell receptor, or the CAR are circulating in the blood. In some embodiments, administered modified lymphocytes in the cell formulation produce a population of progeny cells, wherein the population of progeny cells comprises at least 1×10⁶, at least 1×10⁹, or between 1×10⁶ and 1×10¹¹ modified T cells and/or NK cells. In some embodiments, at least 100 of the administered modified lymphocytes in the cell formulation or their progeny remain localized subcutaneously for at least 7, 14, 21, or 28 days.

Provided herein in another aspect, is a cell formulation comprising an aggregate(s) of T cells and/or NK cells, wherein the T cells and/or NK cells are modified with a polynucleotide comprising one or more transcriptional units, wherein each of the transcriptional units is operatively linked to a promoter active in T cells and/or NK cells, and wherein the one or more transcriptional units encode a first polypeptide comprising a CAR,
and further wherein the aggregate comprises at least 4, 5, 6, or 8 T cells and/or NK cells, wherein the cell aggregate is at least 15 μm in its smallest dimension, and/or wherein the cell aggregate is retained by a coarse filter having a pore diameter of at least 15 μm.

Provided herein in one aspect is a method for engrafting genetically modified lymphocytes in a subject, comprising
a) administering a solution comprising modified lymphocytes to the subject subcutaneously, wherein the modified lymphocytes are modified by either or both, being associated with a RIP comprising a polynucleotide comprising one or more transcriptional units, wherein each transcriptional unit is operatively linked to a promoter active in T cells and/or NK cells, or by being genetically modified with the polynucleotide, wherein the one or more transcriptional units encode a first polypeptide comprising a CAR and typically a second polypeptide comprising an LE; and
b) incubating the modified lymphocytes subcutaneously for at least 0.5, 1, 2, 3, 4, or 8 hours such that least some of the modified lymphocytes are genetically modified with the polynucleotide, or until at least 10%, 20%, 25%, 30%, 40% or 50% of the modified lymphocytes are genetically modified with the polynucleotide. In illustrative embodiments, the genetically modified T cells and/or NK cells are capable of survival in ex vivo culture for at least 7 days in the absence of a target for an antigen-specific targeting region of the CAR and in the absence of exogenous cytokines.

In any of the aspects provided herein that include intramuscular, and in illustrative embodiments subcutaneous administration, of modified lymphocytes (e.g. modified T cells and/or NK cells), in certain embodiments, the subcutaneous administration is performed on a mammalian subject in a method that does not require lymphodepletion of the subject for successful engraftment in the subject and/or for successful reduction of tumor volume in the subject, or that is performed on a mammalian (e.g. human) subject that has not been subjected to lymphodepletion in the prior 1, 2, 3, 4, 5, 6, or 7 days, or prior 1, 2, 3 or 4 weeks, or prior 1, 2, 3, 6, 9, 12, or 24 months or ever before such subcutaneous administration. In certain embodiments, the subcutaneous administration is performed on a mammalian (e.g. human) subject that is not suffering from a low white blood cell count, lymphopenia or lymphocytopenia. In certain embodiments, the subcutaneous administration is performed on a subject having a lymphocyte count in the normal range (i.e., 1,000 and 4,800 lymphocytes in 1 microliter (μL) of blood). In certain embodiments, the subcutaneous administration is performed on a subject having between 1,000 and 5,000, over 300, over 500, over 1,000, over 1,500, or over 2,000 lymphocytes per μL of blood). In certain embodiments, the subcutaneous administration is performed on a mammalian (e.g., human) subject that is lymphoreplete.

In any of the aspects provided herein that include intramuscular, and in illustrative embodiments subcutaneous administration of modified lymphocytes (e.g. modified T cells and/or NK cells), such method can in certain embodiments, include a step wherein the modified cells expand subcutaneously (e.g. expanding the modified cells subcutaneously), for example at or near (e.g. within 10, 5, 4, 3, 2, or 1 cm) a site of subcutaneous administration, for days (e.g., for up to 5, 7, 14, 17, 21, or 28 days) or months (e.g., for up to 1, 2, 3, 6, 12, or 24 months). In some embodiments of aspects herein that include intraperitoneal, intramuscular, and in illustrative embodiments subcutaneous administration of modified T cells and/or NK cells, or RIPs to modify T cells and/or NK cells in vivo, the modified T cells and/or NK cells (e.g., genetically modified T cells and/or NK cells) migrate away from the site of subcutaneous administration to other sites of the body, for example to tumors. Thus, in some embodiments modified and in illustrative embodiments, such methods can include a step wherein genetically modified T cells and/or NK cells appear in circulation migrating away from a subcutaneous administration site, days (e.g., 1, 2, 3, 4, 5, 6, or 7 days), weeks (e.g., 1, 2, 4, or 4 weeks), or months (e.g., 1, 2, 3, 6, 12, or 24 months) after the modified T cells are injected intraperitoneally, intramuscularly, or in illustrative embodiments subcutaneously into a subject. In certain embodiments, at these timepoints, such methods can include a step wherein an area, or in illustrative embodiments a concentration gradient of modified, and in illustrative embodiments genetically modified, T cells and/or NK cells forms emanating from the site of intramuscular, or in illustrative embodiments subcutaneous administration.

In any of the aspects provided herein that include intraperitoneal, intramuscular, and in illustrative embodiments subcutaneous administration of modified lymphocytes (e.g. modified T cells and/or NK cells), certain embodiments can include a step of delivery of another component(s) such as a molecule(s) (ion(s)), macromolecule(s) (e.g. DNA, RNA, peptides, and polypeptides) and/or other cell(s), such as other modified cell(s) (e.g. genetically modified cell(s)), that can affect the modified T cells and/or NK cells, subcutaneously at or near the site of delivery of the modified T cells and/or NK cells, in the same or a different formulation. In certain illustrative embodiments, the other component(s) include an antigen, a recombinant cell encoding a recombinant antigen, or an RNA encoding the antigen, or a cytokine that drives proliferation of T cells and/or NK cells. These other components, which are disclosed in more detail herein, can be delivered either in the same formulation or in different formulation(s) than the modified T cells and/or NK cells. Furthermore, these other components can be delivered along with the modified T cells and/or NK cells or can be delivered days (e.g., 1, 2, 3, 4, 5, 6, or 7 days), weeks (e.g., 1, 2, 4, or 4 weeks), or even months (e.g., 1, 2, 3, 6, 12, or 24 months) before or after the modified T cells and/or NK cells are delivered. In some embodiments, one or more of these other components is delivered at more than one time point, such as on the same day as, or simultaneously with the modified T cells and/or NK cells, and at one or more of the times recited hereinabove in this paragraph. Accordingly, in some embodiments a second formulation is administered to the subject at a second timepoint between 1 day and 1 month, 2 months, 3 months, 6 months, or 12 months after the administering the cell formulation. The other component that is adminsistered to the subject in addition to modified lymphocytes or substantially purified or purified RIPs, can include (e.g. i) a cytokine, for example IL-2, ii) an antibody or polypeptide that is capable of binding CD2, CD3, CD28, OX40, 4-1BB, ICOS, CD9, CD53, CD63, CD81, and/or CD82, and/or iii) a source of the cognate antigen recognized by the CAR). In certain embodiments subcutaneous administration of the modified T cells and/or NK cells is performed near (e.g., within 1, 1, 2, 3, 4, 5, 10, 20, or 30 cm) a site of neoplastic (e.g., cancerous) cells, such as a tumor, or an organ comprising a tumor, including for example, the spleen in the case of blood cancers, or where multiple administrations are performed of the same formulation, or of different formulations, they can be performed at or near the site of a prior administration or away from such site. In some embodiments, the cell formulation comprises a source of a cognate antigen for the CAR, wherein the source of the cognate antigen is the cognate antigen, an mRNA encoding the cognate antigen, or a cell expressing the cognate antigen. In some embodiments, the cell formulation comprises a cytokine and wherein the cytokine is IL-2, IL-7, IL-15, or IL-21 or a modified version of any of these cytokines that is capable of binding to and activating a native receptor for the cytokine. The cognate antigen for this and any embodiments herein, including in this Exemplary Embodiments section, can be any of the tumor associated or tumor specific antigens provided herein.

In certain aspects, provided herein is a population of genetically modified T cells and/or NK cells, wherein the populations is in a subcutaneous environment in a mammal, such as a human, wherein at least 50%, 75%, 90%, 95%, 96%, 97%, 98%, or 99% of the modified T cells and/or NK cells are genetically modified, and in illustrative embodiments comprise a polynucleotide encoding a CAR integrated into their genomic DNA. In some embodiments, such a population can further comprise a gradient of the modified T cells and/or NK cells emanating from a site of intramuscular, and in illustrative embodiments subcutaneous delivery, which in some embodiments is formed days (e.g., 1, 2, 3, 4, 5, 6, or 7 days), weeks (e.g., 1, 2, 4, or 4 weeks), or months (e.g., 1, 2, 3, 6, 12, or 24 months) after the modified T cells are injected intramuscularly, or in illustrative embodiments, subcutaneously into a subject. In some embodiments another component(s) such as a molecule(s) (ion(s)), macromolecule(s) (e.g., DNA, RNA, peptides, and polypeptides) and/or other cell(s), such as other modified (e.g., genetically modified) cell(s) that can affect the modified T cells and/or NK cells, as disclosed herein, is present in the subcutaneous environment. The subcutaneous environment can be, for example, 1, 2, 3, 4, 5, 6, 7, 8, 9, or 10 cm³ in volume. In some embodiments, such subcutaneous environment can comprise at least 1×10¹⁰, 1×10¹¹, 1×10¹², or 1×10¹³ genetically modified T cells and/or NK cells, or between 1×10⁹ and 1×10¹⁵ genetically modified T cells and/or NK cells, for example between 1×10¹⁰ and 1×10¹³, between 1×10¹⁰ and 1×10¹², or between 1×10¹¹ and 1×10¹³ genetically modified T cells and/or NK cells. In some embodiments, such subcutaneous environment can comprise between 1×10⁹ and 1×10¹³, for example between 1×10¹⁰ and 1×10¹³, between 1×10¹⁰ and 1×10¹², or between 1×10¹¹ and 1×10¹³ genetically modified T cells and/or modified NK cells wherein such cells comprise a polynucleotide encoding a CAR integrated into their genomic DNA. In a related aspect, provided is a mammalian subject, for example a human subject, wherein at least 50, 60, 70, 75, 80, 90, or 95% of the genetically modified T cells and/or NK cells in the subject that express a CAR, are subcutaneous, and in illustrative embodiments, are the population of genetically modified T cells and/or NK cells in a subcutaneous environment disclosed in this paragraph. In some embodiments, such mammalian subject is located outside a hospital. In some embodiments, such mammalian subject was not subjected to lymphodepletion in the prior 1, 2, 3, 4, 5, 6, or 7 days, or prior 1, 2, 3 or 4 weeks, or prior 1, 2, 3, 6, 9, 12, or 24 months or ever before.

Provided herein in one aspect is a cell formulation, comprising modified T cells and/or NK cells, wherein the modified T cells and/or NK cells are suspended in a delivery solution and are either or both,
i) genetically modified with a polynucleotide comprising one or more transcriptional units, wherein each of the one or more transcriptional units is operatively linked to a promoter active in T cells and/or NK cells, or
ii) associated with a RIP comprising the polynucleotide, wherein the one or more transcriptional units encode a first polypeptide comprising a CAR, and wherein the cell formulation in illustrative embodiments is contained within a syringe, and has a volume of between 0.5 ml and 20 ml, or 2 ml and 10 ml, or another subcutaneous or intramuscular cell formulation volume provided herein, and further comprises at least one of, for example two or more of, neutrophils, B cells, monocytes, basophils, and eosinophils. In illustrative embodiments, the cell formulation is compatible with, effective for, and/or adapted for intramuscular delivery and in further illustrative embodiments subcutaneous delivery.

In some embodiments of any of the aspects that are or include a cell formulations herein, and any reaction mixture embodiments, especially that that include a subcutaneous, intramuscular reaction, or intraperitoneal reaction mixture, the cell formulation or the reaction mixture further comprises i) a cytokine, ii) an antibody, antibody mimetic, or polypeptide that is capable of binding CD3, CD28, OX40, 4-1BB, ICOS, CD9, CD53, CD63, CD81, and/or CD82, and/or iii) a source of the cognate antigen recognized by the CAR.

In any of the cell mixture, cell formulation, or delivery solution aspects or embodiments provided herein, the cell mixture, cell formulation, or delivery solution can include one or more of:
a. the polynucleotide is extrachromosomal in at least 10%, 25%, 50%, 75%, 80%, 90% or 95% of the modified lymphocytes;
b. at least 25%, 50%, 75%, 80%, 90% or 95% of the modified T cells and/or NK cells in the cell formulation do not express one or more of the CAR or a transposase
c. at least 25% 50%, 75%, 80%, 90% or 95% of the modified T cells and/or NK cells in the cell formulation comprise a recombinant viral reverse transcriptase or a recombinant viral integrase;
d. at least 25%, 50%, 75%, 80%, 90% or 95% of the modified T cells and/or NK cells in the cell formulation do not have the polynucleotide stably integrated into their genomes;
e. between 1% and 20%, or optionally between 5% and 15% of T cells and/or NK cells in the cell formulation are genetically modified;
f. at least 25%, 50%, 75%, 80%, 90% or 95% of the modified T cells and/or modified NK cells in the cell formulation are viable; and/or
g. at least 10%, 20%, 30%, 40%, 50% of the modified lymphocytes comprise a viral pseudotyping element and/or a T cell activating antibody on their surface.

Provided herein in another aspect, is a method for preparing a cell formulation, comprising
a) optionally collecting blood comprising lymphocytes from the subject;
b) contacting blood cells comprising the T cells and/or NK cells ex vivo in a reaction mixture comprising a T cell and/or NK cell activation element, with the RIPs, wherein the RIPs comprise
   i) a binding polypeptide and a fusogenic polypeptide on the surface of the retroviral particles, wherein the binding peptide is capable of binding to a T cell and/or NK cell, and wherein the fusogenic polypeptide is capable of mediating fusion of a retroviral particle membrane with a T cell and/or an NK cell membrane; and
   ii) a polynucleotide comprising one or more transcriptional units, wherein each of the one or more transcriptional units is operatively linked to a promoter active in T cells and/or NK cells, wherein the one or more transcriptional units encode a first polypeptide comprising a CAR,
   wherein said contacting facilitates association of the T cells and/or NK cells with the RIPs, and wherein the recombinant retroviral particles modify the T cells and/or NK cells;
b) collecting the modified T cells and/or NK cells in a delivery solution to form a cell formulation comprising a suspension of the modified T cells and/or NK cells; and
c) transferring 0.5 ml and 20 ml, or 2 ml and 10 ml, or another subcutaneous or intramuscular cell formulation volume provided herein, of the cell formulation into a syringe.

Additional cell formulation aspects and embodiments are provided below and in the Detailed Description herein, outside this Exemplary Embodiments section. Various volumes of cell formulations are provided herein for any cell formulation aspect. In some embodiments, the cell formulation is 3 ml or greater in volume, for example 3 ml to 600 ml in volume, or between 50 ml and 500 ml, or between 100 ml and 500 ml. In some embodiments, the cell formulation comprises hyaluronidase. In some embodiments, the cell formulation is 1 ml to 10 ml, 1 ml to 5 ml, 1 ml to 3 ml or 10, 5, 4, 3, or 2 ml or less, or less than 3 ml, or any of the Small Volume Elements provided herein. In illustrative embodiments, the cell formulation does not comprise hyaluronidase. Other volumes and formulations are provided herein. In some embodiments for any of the cell formulation aspects herein, the cell formulation is contained within a syringe. In some embodiments, the cell formulation, for any cell formulation provided herein, is in an incubation bag or a blood processing bag. In illustrative embodiments, the syringe is made using Good Manufacturing Practice (GMP) and is GMP grade and quality.

In some embodiments of any of the cell formulation aspects provided herein, the cell formulation is localized subcutaneously or intramuscularly, or most of the cell formulation is localized subcutaneously or intramuscularly, in a subject. In some embodiments, the cell formulation further comprises a source of the antigen recognized by the CAR. In some embodiments, the modified lymphocytes are products of a method for modifying lymphocytes provided herein.

In any of the aspects herein, the reaction mixture can comprise at least 10%, 20%, 25%, 50%, 75%, 80%, 90%, 95%, or 99% unfractionated whole blood and optionally an effective amount of an anticoagulant, or the reaction mixture can further comprise at least one additional blood or blood preparation component that is not a PBMC, and in further illustrative embodiments such blood or blood preparation component is one or more of the Noteworthy Non-PBMC Blood or Blood Preparation Components provided herein.

In another aspect, provided herein is a reaction mixture, comprising RIPs, a T cell activation element, and blood cells, wherein the recombinant retroviral particles comprise a pseudotyping element on their surface, wherein the blood cells comprise T cells and/or NK cells, wherein the RIPs comprise a polynucleotide comprising one or more nucleic acid sequences, typically transcriptional units operatively linked to a promoter active in T cells and/or NK cells, wherein the one or more transcriptional units encode a first polypeptide comprising a CAR, a first polypeptide comprising an LE, and/or one or more inhibitory RNA molecules, and wherein the reaction mixture comprises at least 10%, 20%, 25%, 50%, 75%, 80%, 90%, 95%, or 99% unfractionated whole blood. The one or more inhibitory RNA molecule(s) can be directed against any target provided herein, including, but not limited to, in this Exemplary Embodiments section or in the Inhibitory RNA Molecules section herein.

In one aspect, provided herein is a reaction mixture, comprising RIPs, and blood cells, wherein the recombinant retroviral particles comprise a pseudotyping element on their surface, wherein the blood cells comprise T cells and/or NK cells, and wherein the reaction mixture comprises at least 10%, 20%, 25%, 50%, 60%, 70%, 75%, 80%, 90%, 95%, or 99% unfractionated whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein.

In another aspect, provided herein is a reaction mixture, comprising RIPs, a T cell activation element, and blood cells, wherein the recombinant retroviral particles comprise a pseudotyping element on their surface, wherein the blood cells comprise T cells and/or NK cells, wherein the RIPs comprise a polynucleotide comprising one or more nucleic acid sequences, typically transcriptional units operatively linked to a promoter active in T cells and/or NK cells, wherein the one or more transcriptional units encode a first polypeptide comprising a CAR, a first polypeptide comprising an LE, and/or one or more inhibitory RNA molecules, and wherein the reaction mixture comprises at least 10%, 20%, 25%, 50%, 75%, 80%, 90%, 95%, or 99% unfractionated whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein. The one or more inhibitory RNA molecule(s) can be directed against any target provided herein, including, but not limited to, in this Exemplary Embodiments section or in the Inhibitory RNA Molecules section herein.

In another aspect, provided herein is a method for modifying and in illustrative embodiments genetically modifying T cells and/or NK cells in blood or a component thereof, comprising contacting blood cells comprising the T cells and/or NK cells ex vivo, with RIPs in a reaction mixture, wherein the RIPs comprise a pseudotyping element on their surface, wherein said contacting facilitates association of the T cells and/or NK cells with the RIPs, wherein the recombinant retroviral particles genetically modify and/or transduce the T cells and/or NK cells, and wherein the reaction mixture comprises at least 10% 10%, 20%, 25%, 50%, 60%, 70%, 75%, 80%, 90%, 95%, or 99% unfractionated whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein

In another aspect, provided herein is use of RIPs in the manufacture of a kit for modifying and in illustrative embodiments genetically modifying T cells and/or NK cells of a subject, wherein the use of the kit comprises: contacting blood cells comprising the T cells and/or NKs cell ex vivo in a reaction mixture, with the RIPs, wherein the RIPs comprise a pseudotyping element on their surface, wherein said contacting facilitates association of the T cells or NK cells with the RIPs, wherein the recombinant retroviral particles genetically modify and/or transduce the T cells and/or NK cells, and wherein the blood cells comprise T cells, NK cells, and wherein the reaction mixture comprises at least 10%, 20%, 25%, 50%, 60%, 70%, 75%, 80%, 90%, 95%, or 99% unfractionated whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein.

The one or more Noteworthy Non-PBMC Blood or Blood Preparation Components are present in certain illustrative embodiments of any of the reaction mixture, use, modified and in illustrative embodiments genetically modified T cell or NK cell, or method for modifying T cells and/or NK cells provided herein, including but not limited to those provided in this Exemplary Embodiments section, because in these certain illustrative embodiments, the reaction mixture comprises at least 10% whole blood. In certain embodiments of any of the aspects herein that include a reaction mixture, the reaction mixture comprises between 10%, 15%, 20%, 25%, 30%, 40%, 50%, 60%, 70%, and 75% on the low end of the range, and 80%, 90%, 95%, 96%, 97%, 98%, 99%, 99.9%, or 99.99% on the high end of the range of whole blood, or at least 10%, 15%, 20%, 25%, 30%, 40%, 50%, 60%, 70%, 75%, 80%, 90%, 95%, 96%, 97%, 98%, 99%, 99.9%, or 99.99% unfractionated whole blood.

In another aspect herein, provided herein is a method comprising administering any of the replication incompetent recombinant retroviral particles (RIPs) provided herein, typically along with an activation element, in illustrative embodiments associated with a membrane of the RIP, directly to a subject, for example by intravenous, intramuscular, intratumor, intraperitoneal, and in illustrative embodiments subcutaneous administration. Such RIPs typically along with an activation element can be administered in a modifying composition. In such methods, any of the contacting steps provided herein can occur in vivo. Thus, in these embodiments, the contacting typically occurs in vivo, in some embodiments with unaltered, naturally occurring target cells (e.g. T cells and/or NK cells) present within the subject, for example that are recruited to the site of the administering. In such embodiments, RIPs can be formulated in any of the delivery solutions and any of the volumes provided herein to form the modifying composition. Such delivery can further include administering a cell suspension to the subject, wherein the cell suspension comprises T cells and/or NK cells, at or near the site on the subject of administering the RIPs, or at a different site.

Accordingly, in some embodiments, provided herein is a method, or in related aspects use of replication incompetent recombinant retroviral particles in the manufacture of a kit for modifying and/or genetically modifying T cells and/or NK cells subcutaneously in a subject, wherein the method or the use of the kit comprises:

administering to the subject, in illustrative embodiments subcutaneously, a modifying composition comprising replication incompetent recombinant retroviral particles (RIPs) and an activation element, wherein the RIPs comprise a polynucleotide encoding a first polypeptide comprising a transgene, and in illustrative embodiments an antigen, an engineered T cell receptor, or a chimeric antigen receptor (CAR),

wherein the modifying composition has a volume between 0.5 ml and 10 ml contained within a syringe, wherein said administering facilitates association of the T cells and/or NK cells with the RIPs, wherein the T cells and/or NK cells are present in the subcutaneous region of the subject, and wherein the RIPs modify the T cells and/or NK cells to form a population of modified T cells and/or NK cells the modifying composition.

In some embodiments, provided herein is a method, or in related aspects use of replication incompetent recombinant retroviral particles in the manufacture of a kit for modifying and/or genetically modifying T cells and/or NK cells subcutaneously in a subject, wherein the use further comprises administering a cell suspension to the subject subcutaneously, wherein the administering the cell suspension has a volume between 2 ml and 25 ml contained within a syringe, wherein the cell suspension comprises T cells and/or NK cells, wherein the RIPs in the modifying composition contact the T cells and/or NK cells, thereby modifying and/or genetically modifying the T cells and/or NK cells in the cell suspension. The cell suspension can comprise T cells and/or NK cells collected earlier from the subject or allogeneic T cells and/or NK cells.

In some embodiments, the modifying composition and the cell suspension are administered within 0.5, 1, 2, 3, 4, or 5 cm of each other on the surface of the skin of the subject. In some embodiments, the administering the cell suspension occurs simultaneously or within 1, 2, 3, 4, 5, 10, 15, 30, 45, or 60 minutes or 1, 2, 3, 4, 5, 6, 7, or 8 hours of the administering the modifying composition. In some embodiments, the cell suspension comprises whole blood collected from the subject. In some embodiments, the cell suspension comprises neutrophils from the subject. In some embodiments, the whole blood has been subjected to a PBMC and TNC enrichment procedure.

In some embodiments, the modifying composition and the cell suspension are contained within the same syringe. In some embodiments, the activation element is a T cell activation element. In some embodiments, the T cell activation element is a polypeptide capable of binding CD3 or any of the T cell activation elements provided herein. In some embodiments, the activation element is on the surface of the replication incompetent recombinant retroviral particles. In some embodiments, the population of modified T cells and/or NK cells comprise a persisting population of genetically modified T cells and/or NK cells, wherein the persisting population of genetically modified T cells and/or NK cells persists in the subject for at least 7, 14, 21, or 28 days or 1, 2, 3, 4, 5, 6, 7, 8, 9, 10, 11, or 12 months or 1, 2, 3, 4, or 5 years after the administering the modifying composition.

In some embodiments, at least 10% of the modified T cells and/or NK cells in the population of modified T cells and/or NK cells, which in illustrative embodiments for these aspects are formed in vivo, are in cell aggregates according to any of the cell aggregate embodiments provided herein. In some embodiments, at least 50% of the CD4+ and/or CD8+ cells in the population of modified T cells and/or NK cells are CD3− according to any of the Dimmed T Cell Characteristics or Dimmed NK Cell Characteristics provided herein.

In one aspect, provided herein is a method for determining an amount of a gene vector, such as a virus like particle or a viral particle, such as a replication incompetent viral particle preparation to dim surface expression of a surface polypeptide by a target dimming percentage on target cells in a dimming volume, comprising:
a) forming a plurality of reaction mixtures comprising a plurality of concentrations of the gene vector preparation and a fixed amount of a target cell suspension, wherein at least two of the reaction mixtures in the plurality of reaction mixtures comprise different concentrations of the gene vector preparation and/or the target cell suspension, wherein the target cell suspension comprises a plurality of cells comprising the surface polypeptide on their surfaces, and wherein the gene vector preparation comprises a plurality of gene vectors comprising a binding polypeptide on their surfaces capable of binding the surface polypeptide;
b) incubating the reaction mixtures for a target dimming time, wherein during the incubating the gene vectors contact the target cells; and
c) measuring the surface expression of the surface polypeptide in the reaction mixture and in a control sample that expresses the surface polypeptides but is not contacted with the gene vector preparation, and/or measuring the surface expression of the surface polypeptide and another surface polypeptide known to be expressed on all the target cells of the target cell suspension; and
d) determining the amount of the gene vector preparation to dim the target dimming percentage of cells in the dimming volume using the measured surface expression of the surface polypeptide in the reaction mixtures, the measured surface expression of the surface polypeptide in the control amount or the measured surface expression of the other surface polypeptide, and the amounts, such as volumes or dilutions, of the gene vector preparation and the target cell suspension in the reaction mixtures.

In some embodiments, the control sample is an amount, such as a volume and/or dilution, of the target cell suspension. In some embodiments, the control sample is a normal blood cell suspension, in some embodiments from a healthy donor.

In another aspect, provided herein is a method for determining the amount of a viral particle preparation, such as a replication incompetent viral particle, to add to a T cell suspension, comprising:

determining the dimming units of the viral particle preparation, wherein viral particles of the viral particle preparation express a binding polypeptide on their surface, and wherein the dimming units are the amount, such as the volume and/or dilution, of the viral particle preparation that reduces an expression of a target surface polypeptide recognized by the binding polypeptide, by a target percentage in a target amount, such as a volume or a dilution, of a control cell suspension, or an on-test cell suspension not contacted with the viral particle preparation, or as compared to another T cell surface marker, such as CD4 or CD8, on an on-test or sample T cell suspension, under contacting conditions, wherein the amount of the viral particle preparation to add is determined by the dimming units of the viral particle preparation and a target dimming percent of the surface polypeptide on the T cell suspension.

In some embodiments, the amount of the viral particle preparation to add is determined by the dimming units of the viral particle preparation, the target dimming percent of the surface polypeptide on the T cell suspension, and an approximate, estimated, calculated and/or empirically-determined concentration of the surface polypeptide in the T cell suspension. In some embodiments, the concentration of the surface polypeptide on the T cell suspension under the contacting conditions is empirically determined using a control cell suspension that expresses a determined or known amount of the surface polypeptide, and wherein the amount of the viral particle preparation to add is determined by the dimming units of the viral particle preparation, the concentration of the surface polypeptide in the T cell suspension, and a target dimming percent of the surface polypeptide on the T cell suspension.

In another aspect, provided herein is a method for determining the amount, binding capacity, or transduction capacity of a gene vector encapsulated in a membrane (e.g. gene vector particle preparation), such as a virus like particle or a viral particle, such as a replication incompetent viral particle preparation to add to a target cell suspension, such as a target blood cell suspension, for example a T cell suspension or an NK cell suspension, comprising:

determining the dimming units of the gene vector, virus like particle, or viral particle preparation under dimming conditions comprising a reaction mixture, wherein gene vectors or viral particles of the gene vector or viral particle preparation express a binding polypeptide on their surface, and wherein dimming units are the amount or volume of the gene vector, virus like particle, or viral particle preparation that reduces a target surface polypeptide by a target percentage in a target volume (e.g. 10, 5, 4, 3, 2, 1, 0.5, 0.1 ml) of a control cell suspension expressing the surface polypeptide, such as a normal blood cell suspension in illustrative embodiments, a heparinized peripheral blood preparation from a healthy donor or a same on-test blood preparation to which the gene vector will be contacted, or as compared to another surface marker in a target cell population, such as an on-test or sample cell suspension, that expresses the surface polypeptide, under contacting conditions, for example after a target contacting/incubating time, for example 12 hours, 10 hours, 8 hours, 6 hours, 4 hours, 2 hours, 1 hour, 30 minutes 15 minutes, 10 minutes 5 minutes, 1 minute contacting/incubating or just contacting and no incubating, at a target temperature, for example 20C, 22C, 25C, or 37C and percent CO2, for example, 4% 5%, or 6%, wherein the amount of the gene vector (e.g. viral particle) preparation to add is determined by the dimming units of the gene vector (viral particle) preparation and a target dimming percent of the surface polypeptide on the target cell (e.g. T cell) suspension.

In some embodiments, the amount of the gene vector (e.g. viral particle) preparation to add is determined by the dimming units of the gene vector (e.g. viral particle) preparation, the target dimming percent of the surface polypeptide on the target cell (e.g. T cell) suspension, and an approximate, estimated, calculated and/or empirically-determined concentration of the surface polypeptide on the T cell suspension.

In some embodiments, the concentration of the surface polypeptide on the gene vector (e.g. T cell) suspension under the contacting conditions is empirically determined using a control cell suspension that expresses a determined or known amount of the surface polypeptide, and wherein the amount of the gene vector (e.g. viral particle) preparation to add is determined by the dimming units of the viral particle preparation, the concentration of the surface polypeptide in the target cell (e.g. T cell) suspension, and a target dimming percent of the surface polypeptide on the target cell (e.g. T cell) suspension.

In some embodiments, the gene vector preparation is a replication incompetent recombinant retroviral particle preparation. In some embodiments, the target dimming percentage is 50%. In some embodiments, the dimming volume is 1 ml. In some embodiments, the surface polypeptide is CD3D, CD3E, CD3G, CD3Z, TCRα, TCRβ, CD16A, NKp46, 2B4, CD2, DNAM, or NKG2D. In some embodiments, the surface polypeptide is CD3D, CD3E, CD3G, TCRα, or TCRβ.

In some embodiments, the binding polypeptide is an activation element. In some embodiments, the activation element is an anti-CD3 antibody.

In some embodiments, the reaction mixtures are incubated for between 2 and 6 hours before measuring the surface expression of the surface polypeptide. In some embodiments, the reaction mixtures are incubated with at 37° C. and 5% CO2. In some embodiments, the measuring the surface expression of the surface polypeptides comprises using a fluorescence-activated cell sorting method. In some embodiments, the measuring the surface expression of the surface polypeptides comprises using a CD3 antibody, and in illustrative embodiments comprises using a CD4 and/or a CD8 antibody to measure the other surface polypeptide. In some embodiments, the CD3 antibody is Anti-Human CD3-Clone SK7, for example. anti-CD3-PerCP (SK7) (BD, 347344), and in illustrative embodiments the anti-CD8 antibody is SK1, for example anti-CD8-FITC (SK1) (BD, 347313).

Provided herein in another aspect is a method for modifying, genetically modifying, and/or transducing a lymphocyte (e.g. a T cell or an NK cell) or a population thereof, comprising contacting blood cells comprising the lymphocyte (e.g. the T cell or NK cell) or the population thereof, ex vivo with a RIP comprising in its genome a polynucleotide comprising one or more nucleic acid sequences operatively linked to a promoter active in lymphocytes (e.g. T cells and/or NK cells), wherein a first nucleic acid sequence of the one or more nucleic acid sequences encodes a CAR comprising an ASTR, a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates genetic modification and/or transduction of the lymphocyte (e.g. T cell or NK cell), or at least some of the lymphocytes (e.g. T cells and/or NK cells) by the RIP, thereby producing a modified, genetically modified, and/or transduced lymphocyte (e.g. T cell and/or NK cell). In such method, the contacting is typically performed in a reaction mixture, sometimes referred to herein as a transduction reaction mixture, comprising a population of lymphocytes (e.g., T cells and/or NK cells) and contacted with a population of RIPs. Various contacting times are provided herein, including, but not limited to, in this Exemplary Embodiments section, that can be used in this aspect to facilitate membrane association, and eventual membrane fusion of the lymphocytes (e.g., T cells and/or the NK cells) to the RIPs.

Provided herein in one aspect, is use of RIPs in the manufacture of a kit for modifying lymphocytes (e.g. T cells or NK cells) of a subject, wherein the use of the kit comprises: contacting blood cells comprising the lymphocytes (e.g. T cells and/or the NK cells) ex vivo in a reaction mixture, with the RIPs, wherein the RIPs comprise a pseudotyping element on their surface, wherein the RIPs comprise a polynucleotide comprising one or more nucleic acid sequences, typically transcriptional units operatively linked to a promoter active in lymphocytes (e.g. T cells and/or NK cells), wherein the one or more transcriptional units encode a first polypeptide comprising a CAR, a first polypeptide comprising an LE, or a first polypeptide comprising an LE and a second polypeptide comprising a CAR, thereby producing the modified and in illustrative embodiments genetically modified lymphocytes (e.g. modified T cells and/or modified NK cells). Various contacting times are provided herein, including, but not limited to, in this Exemplary Embodiments section, that can be used in this aspect to facilitate membrane association, and eventual membrane fusion of the lymphocytes (e.g., T cells and/or the NK cells) to the RIPs. In an illustrative embodiment, contacting is performed for less than 15 minutes.

Provided herein in another aspect is a RIP for use in a method for modifying a lymphocyte, for example a T cell and/or NK cell, wherein the method comprises contacting blood cells comprising the lymphocyte, for example T cell and/or NK cell, of a subject in a reaction mixture, ex vivo, with a RIP comprising in its genome a polynucleotide comprising one or more nucleic acid sequences operatively linked to a promoter active in T cells and/or NK cells, wherein a first nucleic acid sequence of the one or more nucleic acid sequences encodes a CAR comprising an ASTR, a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates transduction of at least some of the resting T cells and/or NK cells by the RIPs, thereby producing a modified and in illustrative embodiments genetically modified T cell and/or NK cell. Various contacting times are provided herein, including, but not limited to, in this Exemplary Embodiments section, that can be used in this aspect to facilitate membrane association, and eventual membrane fusion of the lymphocytes (e.g., T cells and/or the NK cells) to the RIPs. In an illustrative embodiment, contacting is performed for less than 15 minutes. In some embodiments the method can further include introducing the modified T cell and/or NK cell into a subject. In illustrative embodiments, the blood cells comprising the lymphocyte (e.g., the T cell and/or NK cell) are from the subject, and thus the introducing is a reintroducing. In this aspect, in some embodiments, a population of lymphocytes (e.g., T cells and/or NK cells) are contacted in the contacting step, modified, genetically modified, and/or transduced, and introduced into the subject in the introducing step.

Provided herein in another aspect is the use of a RIP in the manufacture of a kit for modifying a lymphocyte, for example a T cell and/or NK cell of a subject, wherein the use of the kit comprises contacting blood cells comprising the lymphocyte, for example the T cell and/or the NK cell of the subject ex vivo in a reaction mixture, with RIPs comprising in their genome a polynucleotide comprising one or more nucleic acid sequences operatively linked to a promoter active in T cells and/or NK cells, wherein a first nucleic acid sequence of the one or more nucleic acid sequences encodes a CAR comprising an ASTR, a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates genetic modification of at least some of the T cells and/or NK cells by the RIPs, thereby producing a modified and in illustrative embodiments genetically modified T cell and/or NK cell. As indicated herein, various contacting times are provided herein, that can be used in this aspect to facilitate membrane association, and eventual membrane fusion of the lymphocyte (e.g. T cell and/or the NK cell) to the RIPs. In an illustrative embodiment, contacting is performed for less than 15 minutes. In illustrative embodiments, the blood cells comprising the lymphocyte (e.g., the T cell and/or NK cell) are from the subject, and thus the introducing is a reintroducing. In this aspect, in some embodiments, a population of T cells and/or NK cells are contacted in the contacting step, modified, genetically modified, and/or transduced, and introduced into the subject in the introducing step.

Provided herein in another aspect is the use of RIPs in the manufacture of a medicament for modifying lymphocytes, for example T cells and/or NK cells of a subject, wherein the use of the medicament comprises:
a) contacting blood cells comprising the T cells and/or NK cells of the subject ex vivo in a reaction mixture, with the RIPs comprising in their genome a polynucleotide comprising one or more nucleic acid sequences operatively linked to a promoter active in T cells and/or NK cells, wherein a first nucleic acid sequence of the one or more nucleic acid sequences encodes a CAR comprising an ASTR, a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates genetic modification of at least some of the lymphocytes (for example, T cells and/or NK cells) by the RIPs, thereby producing modified and in illustrative embodiments genetically modified T cells and/or NK cells; and optionally
b) introducing the modified T cell and/or NK cell into the subject, thereby modifying the lymphocytes, for example T cells and/or NK cells of the subject.

In another aspect, provided herein is kit for modifying NK cells and/or T cells, comprising:
one or a plurality of first containers containing polynucleotides, typically substantially pure polynucleotides (e.g., found within recombinant retroviral particles according to any embodiment herein), comprising a first transcriptional unit operatively linked to a promoter active in T cells and/or NK cells, wherein the first transcriptional unit encodes a first polypeptide comprising a CAR; and one or more additional or accessory components selected from:
a) one or more containers containing a delivery solution compatible with, in illustrative embodiments effective for, and in further illustrative embodiments adapted for subcutaneous and/or intramuscular administration as provided herein;
b) one or more sterile syringes compatible with, in illustrative embodiments effective for, and in further illustrative embodiments adapted for, subcutaneous or intramuscular delivery of T cells and/or NK cells;
c) one or a plurality of leukoreduction filtration assemblies;
d) one or more containers of hyaluronidase as provided herein;
e) one or more blood bags such as a blood collection bag, in illustrative embodiments comprising an anticoagulant in the bag, or in a separate container, a blood processing buffer bag, a blood processing waste collection bag, and a blood processing cell sample collection bag;
f) a T cell activation element as disclosed in detail herein, for example anti-CD3 provided in solution in the container containing the retroviral particle, or in a separate container, or in illustrative embodiments, is associated with a surface of the replication incompetent retroviral particle;
g) one or more containers containing a solution or media compatible with, in illustrative embodiments effective for, and in further illustrative embodiments adapted for transduction of T cells and/or NK cells;
h) one or more containers containing a solution or media compatible with, in illustrative embodiments effective for, and/or in further illustrative embodiments adapted for rinsing T cells and/or NK cells;
i) one or more containers containing a pH-modulating pharmacologic agent;
j) one or more containers containing second polynucleotides, typically substantially pure polynucleotides (e.g. found within recombinant retroviral particles according to any embodiment herein), comprising a second transcriptional unit operatively linked to a promoter active in T cells and/or NK cells, wherein the second transcriptional unit encodes a second polypeptide comprising a second CAR directed against a different target epitope or in certain embodiments a different antigen, in illustrative embodiments found on a same target cancer cell (e.g. B cell);
k) one or more containers containing a cognate antigen for the first CAR and/or the second CAR encoded by the nucleic acids (e.g., retroviral particles); and
l) instructions, either physically or digitally associated with other kit components, for the use thereof, for example for modifying T cells and/or NK cells, for delivering modified T cells and/or NK cells to a subject subcutaneously or intramuscularly, and/or for treating tumor growth or cancer in a subject.

In one aspect, provided herein is a kit for modifying NK cells and/or T cells, comprising: one or a plurality of containers,
wherein at least one of the plurality of containers comprises either i) polynucleotides, and in illustrative embodiments replication incompetent recombinant retroviral particles (RIPs), each encoding a first polypeptide comprising an engineered T cell receptor or a chimeric antigen receptor (CAR), or ii) T cells or NK cells each capable of expressing the CAR,
wherein at least one of the plurality of containers contains one or more additional components selected from: a composition comprising i) a cytokine, ii) a source of the cognate antigen recognized by the CAR, and iii) a target cell depletion agent, and
wherein at least one of the plurality of containers contain a delivery solution adapted for subcutaneous administration, and/or wherein the kit further comprises one or more sterile syringes adapted for subcutaneous delivery of T cells and/or NK cells.

In some embodiments, the kit further comprises a leukoreduction filter assembly.

In another aspect, provided herein is a kit for modifying NK cells and/or T cells, comprising:
one or a plurality of containers,
wherein at least one of the plurality of containers comprises either i) polynucleotides, in illustrative embodiments replication incompetent recombinant retroviral particles (RIPs), each encoding a first polypeptide comprising an engineered T cell receptor or a chimeric antigen receptor (CAR), or ii) T cells or NK cells each capable of expressing the CAR; and
a leukoreduction filter assembly.

In some embodiments, at least one of the plurality of containers contain a delivery solution adapted for subcutaneous administration, and/or wherein the kit further comprises one or more sterile syringes adapted for subcutaneous delivery of T cells and/or NK cells. In some embodiments, at least one of the plurality of containers contains one or more additional components selected from: a composition comprising i) a cytokine, ii) a source of the cognate antigen recognized by the CAR, and iii) a target cell depletion agent.

In some embodiments, the kit further comprises the one or more sterile syringes adapted for subcutaneous delivery of T cells and/or NK cells, and wherein the leukoreduction filter assembly is adapted, configured, and/or effective, to process no more than 100, 50, or 25 ml of blood. In some embodiments, the additional component is the composition comprising the cytokine, and wherein the cytokine does not bind to a cytokine receptor included in the kit or a cytokine receptor that is encoded by a polynucleotide. In some embodiments, the cytokine is IL-2, IL-7, IL-15, or IL-21, or a modified version of any of these cytokines that is capable of binding to and activating a native receptor for the cytokine. In some embodiments, the additional component is the composition comprising the source of the cognate antigen. In some embodiments, the source is a nucleic acid encoding the cognate antigen. In some embodiments, the nucleic acid encoding the cognate antigen is an mRNA. In some embodiments, the source is soluble cognate antigen. In some embodiments, the additional component is the composition comprising the target cell depletion agent.

In another aspect, provided herein is a kit for modifying NK cells and/or T cells, comprising:
one or a plurality of containers,
wherein at least one of the plurality of containers contains i) polynucleotides, and in illustrative embodiments replication incompetent recombinant retroviral particles (RIPs) encoding a first polypeptide comprising an engineered T cell receptor or a chimeric antigen receptor (CAR), wherein the extracellular domain of the CAR includes an epitope tag, or ii) T cells or NK cells capable of expressing the first polypeptide, and
wherein at least one of the plurality of containers contains either a polynucleotide encoding a polypeptide capable of binding and in illustrative embodiments crosslinking the epitope tag or cells that express a polypeptide capable of binding to the epitope tag.

In some embodiments, at least one of the plurality of containers contain a delivery solution adapted for subcutaneous administration, and/or wherein the kit further comprises one or more sterile syringes adapted for subcutaneous delivery of T cells and/or NK cells. In some embodiments, the kit comprises the polynucleotides, wherein the polynucleotides are located within replication incompetent recombinant retroviral particles, and wherein the surface of the replication incompetent recombinant retroviral particles further comprises an activation element, wherein the activation element is capable of activating a T cell and/or an NK cell. In some embodiments, the kit comprises the T cells and/or the NK cells and wherein the T cells and/or the NK cells are allogeneic cells.

In some embodiments, at least one of the plurality of containers contain the delivery solution adapted for subcutaneous administration, and wherein the delivery solution adapted for subcutaneous administration comprises an artificial matrix. In some embodiments, the artificial matrix comprises hyaluronic acid and/or collagen. In some embodiments, at least one of the plurality of containers contains either i) second polynucleotides encoding a second polypeptide comprising a second chimeric antigen receptor (CAR), or ii) a second population of T cells or NK cells capable of expressing a second CAR. In some embodiments, the first CAR is capable of binding to CD19 and the second CAR is capable of binding to CD22. In some embodiments, the additional component comprises the target cell depletion agent, and wherein the target cell depletion agent is a B cell depletion agent, and in illustrative embodiments wherein the B cell depletion agent is not anti-CD19 CAR.

In another aspect, provided herein is a kit for modifying NK cells and/or T cells, comprising:
one or a plurality of containers,
wherein at least one of the plurality of containers contains polynucleotides comprising a first transcriptional unit operatively linked to a promoter active in T cells and/or NK cells, wherein the first transcriptional unit encodes a first polypeptide comprising a chimeric antigen receptor (CAR); and
wherein at least one of the plurality of containers contains one or more additional components selected from: a composition comprising i) a cytokine, and ii) a binding partner for an external epitope of the CAR, or a polynucleotide encoding the binding partner; and
wherein at least one of the plurality of containers contain a delivery solution adapted for subcutaneous administration, and/or wherein the kit further comprises one or more sterile syringes adapted for subcutaneous delivery of T cells and/or NK cells.

In some embodiments, the additional component is the composition comprising the binding partner for the external epitope of the CAR, or the polynucleotide encoding the binding partner. In some embodiments the composition further comprises a source of the cognate antigen recognized by the CAR. In some embodiments, the polynucleotides comprising the first transcriptional unit further encode an epitope and wherein the composition comprising the binding partner for the external epitope of the CAR comprises a source of a polypeptide capable of binding to the epitope.

In another aspect, provided herein is a kit for modifying NK cells and/or T cells, comprising: one or a plurality of containers,
wherein at least one of the plurality of containers contains polynucleotides comprising a first transcriptional unit operatively linked to a promoter active in T cells and/or NK cells, wherein the first transcriptional unit encodes a first polypeptide comprising a chimeric antigen receptor (CAR);
wherein at least one of the plurality of containers contains one or more additional components selected from: a composition comprising i) a cytokine and ii) a source of the cognate antigen recognized by the CAR, or iii) a binding partner for an external epitope of the CAR; and wherein at least one of the plurality of containers contain a delivery solution adapted for subcutaneous administration, and/or wherein the kit further comprises one or more sterile syringes adapted for subcutaneous delivery of T cells and/or NK cells.

In some embodiments, the polynucleotides encoding the CAR are located within replication incompetent recombinant retroviral particles. In some embodiments, the surface of the replication incompetent recombinant retroviral particles further comprises an activation element, wherein the activation element is capable of activating a T cell and/or an NK cell. In some embodiments, the additional component is the composition comprising the binding partner for the external epitope of the CAR. In some embodiments, the binding partner for the external epitope of the CAR comprises the source of the cognate antigen. In some embodiments, the composition the source of the cognate antigen is the cognate antigen, a nucleic acid encoding the cognate antigen, or a cell comprising a nucleic acid encoding the cognate antigen. In some embodiments, the composition comprising the source of the cognate antigen is the nucleic acid encoding the cognate antigen.

In some embodiments, the composition comprising the nucleic acid encoding the cognate antigen comprises an mRNA encoding the cognate antigen. In some embodiments, the composition comprising the source of the cognate antigen comprises soluble cognate antigen. In some embodiments, the polynucleotides comprising the first transcriptional unit further encode an epitope, and wherein the composition comprising the binding partner for the external epitope of the CAR comprises a source of a polypeptide capable of binding to the epitope. In some embodiments, the replication incompetent recombinant retroviral particles further comprise on their surface, a binding polypeptide and a fusogenic polypeptide, wherein the binding polypeptide is capable of binding to a T cell and/or NK cell, and wherein the fusogenic polypeptide is capable of mediating fusion of a retroviral particle membrane with a T cell and/or an NK cell membrane.

In some embodiments, the one or more containers containing the replication incompetent retroviral particles contain substantially-pure, GMP grade, replication incompetent retroviral particles. In some embodiments, each container containing the replication incompetent retroviral particles contains a volume of between 0.1 ml and 10 ml, or a volume of any of the Small Volume Elements herein, and between 1×10⁶ and 5×10⁹ retroviral particle Transducing Units or between 1 and 50 Units, or enough Dimming Units to dim at least 50%, 60%, 70%, 75%, 80%, 90%, 95%, or 99% of the target cells (e.g. T cells).

In some embodiments, the kit comprises one or more containers containing the delivery solution adapted for subcutaneous administration. In some embodiments, the kit comprises one or a plurality of leukoreduction filtration assemblies. In some embodiments, the kit comprises the one or more sterile syringes adapted for subcutaneous delivery of T cells and/or NK cells. In some embodiments, the kit comprises
a) one or more containers containing the delivery solution adapted for subcutaneous administration; and
b) the one or more sterile syringes adapted for subcutaneous delivery of T cells and/or NK cells.

In any of the assembly aspects, or methods or uses that include assemblies, part numbers are sometimes provided as referenced in the figures, as non-limiting examples only, as is the case throughout this Exemplary Embodiments section and this specification. Furthermore, any reference to tubing or a specific type of channel, are as non-limiting examples of a channel.

In one aspect, provided herein is a leukoreduction filtration assembly, comprising:
a) a reaction mixture collection container with a maximum volume of 100 ml, 75 ml, 60 ml, 50 ml, 40 ml, 30 ml, 20 ml, 15 ml, or 10 ml; and
b) a leukoreduction filter;
   In illustrative embodiments, the leukoreduction filter assembly further comprises:
c) a collection valve,
d) wherein an inlet channel (e.g. inlet tubing) connects a first assembly opening to a leukoreduction filter enclosure comprising the leukoreduction filter, wherein a first connection junction between the first assembly opening and the inlet tubing has an angle of between 5° and 80°, 70°, 60°, 50°, 45°, 40°, 30°, 25°, 20°, or 10°, or between 10° and 80°, 70°, 60°, 50°, 45°, 40°, 30°, 25°, 20°, or 15°, or between 15° and 80°, 70°, 60°, 50°, 45°, 40°, 30°, 25°, 20°, or between 20° and 80°, 70°, 60°, 50°, 45°, 40°, 30°, or 25° with respect to the inlet tubing, and the inlet tubing has no junctions that are greater than 80°, 75°, 70°, 65°, 60°, 55°, or 50°, and wherein the leukoreduction filter has an effective filtration area of between 2 cm2 and 5 cm2 or between 3 cm2 and 5 cm2.

In another aspect, provided herein is a method for genetically modifying mammalian nucleated blood cells, comprising:
a) transporting between 5 ml and 125 ml, 120 ml, 100 ml, 50 ml, 5 ml and 40 ml, 5 ml and 30 ml, 5 ml and 25 ml, or between 10 ml and 125 ml, 120 ml, 100 ml, 50 ml, 5 ml and 40 ml, 5 ml and 30 ml, 5 ml and 25 ml, of whole blood comprising whole blood cells into a transduction assembly comprising an incubation bag comprising nucleic acid vector copies to form a reaction mixture, wherein the incubation bag has a maximum volume capacity of 100 ml, 75 ml, 60 ml, 50 ml, 40 ml, 30, 20, or 10 ml, and wherein the incubation bag is connected to an inlet channel (e.g. inlet tubing) at a first assembly opening;
b) contacting the whole blood cells with the vector copies within the reaction mixture to produce modified whole blood cells;
c) transporting the modified whole blood cells through to channel to a reaction mixture collection container;
d) transporting the modified whole blood cells from the reaction mixture collection container to a leukoreduction filter of the leukoreduction filter assembly to filter the modified whole blood cells to produce an enriched fraction of modified nucleated blood cells, wherein the leukoreduction filter has effective filtration area of between 3 cm² and 5 cm²; and
e) collecting between the enriched fraction of modified blood cells in between 0.5 to 20 ml, 15 ml, 10 ml, 5 ml, or 2.5 ml, or between 1 ml to 20 ml, 15 ml, 10 ml, 5 ml, or 2.5 ml of a delivery solution to form a cell formulation comprising a suspension of the modified nucleated blood cells. In illustrative embodiments, at least 10% of the modified T cells and/or NK cells in the cell formulation are aggregated.

In some embodiments of any aspects herein, the collecting is performed by transporting the cell formulation into a syringe. In some embodiments of any aspects herein, the method further comprises administering the cell formulation to a subject subcutaneously at a first subcutaneous site. In some embodiments of any aspects herein, the whole blood is from a subject and the method further comprises collecting whole blood from the subject, which in illustrative embodiments can be collected into a whole blood container. In some embodiments of any aspects herein, the entire method from the collecting whole blood to the administering, not including a duration of the contacting, is completed in between 15 minutes and 1 hour, or between 15 minutes and 45 minutes. In some embodiments of any aspects herein, the entire method from the transporting the whole blood to the collecting the enriched fraction of modified blood cells is completed in between 15 minutes and 1 hour, or between 15 minutes and 45 minutes. In some embodiments of any aspects herein, the entire method from the collecting whole blood to the administering is completed in between 15 minutes and 12 hours, 15 minutes and 10 hours, 15 minutes and 8 hours, 15 minutes and 6 hours, or 15 minutes and 4 hours, or less than 12, 10, 8, 6, 4, 2, or 1 hour.

In some embodiments of any aspects herein, at least 10% of the modified T cells and/or NK cells in the formulation are aggregated at the time they are administered to the subject. In some embodiments of any aspects herein, the whole blood is transported through a collection valve of the transduction assembly through the tubing to the incubation bag before the contacting, wherein the collection valve has an angle of between 5° and 80°, 70°, 60°, 50°, 45°, 40°, 30°, 25°, 20°, or 10° with respect to the inlet tubing on the side of the leukoreduction filter enclosure. In some embodiments of any aspects herein, the collecting is performed by injecting between 0.5 and 20 ml, 0.5 and 10 ml, 1 and 10 ml, or 3 and 7 ml of the delivery solution in the opposite direction of the direction the modified whole blood cells are transported, from a delivery solution syringe connected to outlet tubing that is in fluid communication with the inlet tubing of the leukoreduction filter assembly at an attachment junction on the other side of the leukoreduction filter enclosure with respect to the first assembly opening, wherein the delivery solution syringe has a maximum volume of 25, 20, 25, 10, 7.5, 5, 4, 3, 2 or 1 ml.

In some embodiments of any aspects herein, the method further comprises after filtering the modified nucleated blood cells, washing the modified nucleated blood cells to produce the enriched fraction. In some embodiments of any aspects herein, the washing is performed with between 0.25× and 3×, or 0.75× and 2.5×, or 0.75 and 2× the volume of whole blood transported into the first blood bag. In some embodiments of any aspects herein, the washing is performed using a syringe.

In some embodiments of any aspects herein, the collecting is performed in a cell sample collection bag of the leukoreduction filter assembly connected to the inlet tubing on the same side of the leukoreduction filter enclosure as the first assembly opening. In some embodiments of any aspects herein, the reaction mixture collection container, the cell sample collection bag, and the first assembly opening are configured such that the reaction mixture collection container and the cell sample collection bag can be reversibly connected to the inlet tubing at the first assembly opening. In some embodiments of any aspects herein, the cell formulation comprises neutrophils. In some embodiments of any aspects herein, the leukoreduction filter assembly is inverted prior to collecting such that the enriched fraction of modified blood cells are collected by fluid moving down across the filter. In some embodiments of any aspects herein, the inlet tubing is a continuous tubing that does not comprise any junctions having an angle greater than 80°, 75°, 70°, 65°, 60°, 55°, or 50° in a flow path of the inlet tubing.

In some embodiments of any aspects herein, the leukoreduction filtration assembly further comprises:
i) a reaction mixture collection container with a maximum volume of 50 ml, 40 ml, 30 ml, 25 ml, 20 ml, 15 ml, or 10 ml, or any of the Small Volume Elements reaction mixture volumes provided herein;
ii) a wash buffer syringe with a maximum volume of 100 ml, 75 ml, 50 ml, 40 ml, 30 ml, 25 ml, 20 ml, 15 ml, or 10 ml; and
iii) a second assembly opening,
wherein the reaction mixture collection container and the first connection junction are configured such that the reaction mixture collection container is reversibly connected to the inlet tubing at the first connection junction, and
wherein the second assembly opening is connected to the inlet tubing at an angle of between 5° and 80°, 70°, 60°, 50°, 45°, 40°, 30°, 25°, 20°, or 10°, with respect to the inlet tubing.

In some embodiments of any aspects herein, the leukoreduction filtration assembly further comprises an outlet valve connected to outlet channel (e.g. tubing) that is in fluid communication with the inlet channel (e.g. tubing) at a point on the other side of the leukoreduction filter enclosure from the first assembly opening, wherein a delivery solution syringe that connects to the outlet valve has a maximum volume of 25, 20, 25, 10, 7.5, 5 or 2.5 ml. In some embodiments of any aspects herein, the incubation bag comprises a reaction mixture comprising mammalian nucleated blood cells and nucleic acid vector copies. In some embodiments of any aspects herein, the mammalian nucleated blood cells are T cells, NK cells, CD4+ lymphocytes, CD8+ lymphocytes, CD56+ lymphocytes, B cells, dendritic cells, macrophages, and neutrophils. In some embodiments of any aspects herein, the mammalian nucleated blood cells comprise T cells, NK cells, CD4+ lymphocytes, CD8+ lymphocytes, and/or CD56+ lymphocytes. In some embodiments of any aspects herein, the mammalian nucleated blood cells comprise dendritic cells or macrophages. In some embodiments of any aspects herein, the nucleic acid vector copies comprise a population of replication incompetent recombinant retroviral particles. In some embodiments of any aspects herein, the transgene encodes a polypeptide comprising a chimeric antigen receptor (CAR).

In some embodiments, any of the use or methods provided herein that include a reaction mixture, the use or method comprises or further comprises:
providing a transduction assembly (301) comprising a first assembly opening (317) in fluid communication with optional tubing (354) and an incubation bag (314), a vector container (311), a whole blood container (313), and a reaction mixture collection container (315),
wherein the reaction mixture is formed in the incubation bag (314) by transporting between 1 ml and 20 ml, 15 ml, 10 ml, 7.5 ml, 5 ml, 4 ml, or 3 ml, or between 2 ml and 20 ml, 15 ml, 10 ml, 7.5 ml, 5 ml, 4 ml, or 3 ml of the RIPs through the first assembly opening (317) and the tubing (354) and into the incubation bag (314), and by transporting between 5 ml and 50 ml, 40 ml, 30 ml, 25 ml, 20 ml, 15 ml, or 10 ml, or between 10 ml and 50 ml, 40 ml, 30 ml, 25 ml, 20 ml, or 15 ml, or between 15 ml and 50 ml, 40 ml, 30 ml, 25 ml, or 20 ml of the blood cells comprising lymphocytes are transported through the first assembly opening (317) and the tubing (354) and into the incubation bag (314),
wherein the modified lymphocytes are collected in the reaction mixture collection container (315) before forming the cell formulation by transporting the reaction mixture from the incubation bag (314) through the tubing (354) and first assembly opening (317) and into the reaction mixture collection container (315).

In some embodiments, the use, wherein the cell formulation is formed using a leukoreduction filter assembly (401) comprises the reaction mixture collection container (315) comprising the reaction mixture, a first assembly opening (417) in fluid communication with a collection valve (445), inlet channel (e.g. inlet tubing) (455), a filter enclosure inlet (425), a leukoreduction filter enclosure (410), a filter enclosure outlet (426), outlet channel (e.g. outlet tubing) (456), an outlet valve (446), and a waste collection bag (416), and a cell sample collection bag (465) for collecting the cell formulation with a maximum volume of 100 ml, 75 ml, 60 ml, 50 ml, 40 ml, 30 ml, 20 ml, 15 ml, 10 ml, or 5 ml, wherein the first assembly opening (417) attaches at an angle of between 5° and 80°, 70°, 60°, 50°, 45°, 40°, 30°, 25°, 20°, or 10°, or between 10° and 80°, 70°, 60°, 50°, 45°, 40°, 30°, 25°, 20°, or 15°, or between 15° and 80°, 70°, 60°, 50°, 45°, 40°, 30°, 25°, 20°, or between 20° and 80°, 70°, 60°, 50°, 45°, 40°, 30°, or 25° with respect to the inlet channel (e.g. inlet tubing) (455), and the inlet channel (e.g. inlet tubing) (455) has no junctions that are greater than 80°, 70°, 60°, or 50°, and wherein the leukoreduction filter has an effective filtration area of between 1 cm² and 10 cm², for example, between 2 cm² and 8 cm² or between 3 cm² and 5 cm².

In certain methods using the leukoreduction filter assembly, the use further comprises:
transporting the reaction mixture in the reaction mixture collection container (315) through the first assembly opening (417), inlet tubing (455), and filter enclosure inlet (425) and into the leukoreduction filter enclosure (410), wherein the components of the reaction mixture not retained on the leukoreduction filter pass through the filter enclosure outlet (426) then the outlet tubing (456) and the outlet valve (446) and into the waste collection bag (416);
optionally washing the reaction mixture retained on the leukoreduction filter (410) with a wash solution, wherein the wash solution passes through the filter enclosure outlet (426) and the outlet valve (446) and into the waste collection bag (416);
turning the outlet valve (446) and the collection valve (445) to collection positions;
delivering a volume of delivery solution through the outlet valve (446) into the outlet tubing (456) then through the filter enclosure outlet (426), the leukoreduction filter enclosure (410), the filter enclosure inlet (425), the inlet tubing (455), the collection valve (445), and into the cell sample collection bag (465), wherein the delivering the volume of delivery solution forms the cell formulation.

In some embodiments, the use, wherein the leukoreduction filter assembly (400) further comprises a third assembly opening (420), and the use further comprises collecting the cell formulation from the cell sample collection bag (465) into a cell sample collection syringe (467). In some embodiments, the cell formulation is administered subcutaneously. In some embodiments, the use further comprises collecting whole blood from the subject before transporting the whole blood to the blood bag.

Provided in the following paragraphs, are exemplary aspects and embodiments that can be used in or combined with any aspect or embodiment provided herein unless incompatible with or otherwise indicated, as will be recognized by a skilled artisan. In another aspect, provided herein is a modified, in illustrative embodiments genetically modified, and in further illustrative embodiments stably transfected or stably transcribed lymphocyte(s) (e.g., T cell(s) or NK cell(s)) made by modifying lymphocytes (e.g., T cells and/or NK cells) according to any method herein.

In another aspect, provided herein is use of a RIPs in a kit, or in the manufacture of a kit, for modifying T cells and/or NK cells of a subject, wherein the use of the kit comprises any of the methods for modifying T cells and/or NK cells provided herein. In another aspect, provided herein is use of a RIPs in a kit, or in the manufacture of a kit for delivering to a subject, administering to a subject, injecting into a subject, and/or engrafting in a subject, modified lymphocytes, wherein the use of the kit comprises any of the methods for delivering to a subject, administering to a subject, injecting into a subject, and/or engrafting in a subject, provided herein. In another aspect, provided herein is use of a RIPs in a kit, or in the manufacture of a kit for preparing a cell formulation, wherein the use of the kit comprises any of the methods for preparing a cell formulation comprising modifying T cells and/or NK cells provided herein. Provided herein in another aspect, are RIPs for use in subcutaneous delivery to a subject, wherein the use of the RIPs comprises any method provided herein, for subcutaneous delivery that comprises RIPs.

Provided in the following paragraphs, are exemplary embodiments, for example exemplary ranges and lists, that can be used for any of the aspects provided immediately above or otherwise herein, unless incompatible with or otherwise indicated, as will be recognized by a skilled artisan. Additional aspects and embodiments are provided in this specification outside this Exemplary Embodiments section.

In any of the aspects herein, the cell(s) or lymphocyte(s) is an NK cell(s) or in illustrative embodiments a T cell(s). It will be understood that in aspects that include collecting blood that such method can include collecting a blood-derived product or a peripheral blood-derived product, which can be a blood sample, such as an unfractionated blood sample, or can include blood cells (e.g., leukocytes or lymphocytes) collected by apheresis.

In any of the aspects herein that include a polynucleotide including one or more transcriptional units, the one or more transcriptional units can encode a polypeptide comprising an LE. In some embodiments, the lymphoproliferative element comprises an intracellular signaling domain from a cytokine receptor, which in illustrative embodiments activates a Janus kinase/Signal Transducer and Activator of Transcription (JAK/STAT) pathway and/or a tumor necrosis factor receptor (TNF-R)-associated factor (TRAF) pathway. In illustrative embodiments, the lymphoproliferative element is constitutively active typically because it constitutively activates one or more signaling pathways. In illustrative embodiments, the lymphoproliferative element comprises Box1 and optionally Box2 JAK-binding motifs, and a STAT-binding motif comprising a tyrosine residue. In some illustrative embodiments, the lymphoproliferative element does not comprise an extracellular ligand binding domain or a small molecule binding domain In some embodiments, the constitutively active signaling pathways include activation of PI3K pathways. In some embodiments, the constitutively active signaling pathways include activation of PLC pathways. Thus, in certain embodiments, lymphoproliferative elements for use in any of the kits, methods, uses, or compositions herein, are constitutively active and comprise an intracellular signaling domain that activates a Jak/Stat pathway a TRAF pathway, a PI3K pathway, and/or a PLC pathway. Any of the polypeptide lymphoproliferative elements disclosed herein, for example, but not limited to those disclosed in the “Lymphoproliferative elements” section herein, or functional mutants and/or fragments thereof, can be encoded. In some embodiments, the LE comprises a domain with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids or an intracellular domain from 4-1BB (CD137), B7-H3, B7-HCDR3, BAFFR, BTLA, C100 (SEMA4D), CD2, CD3D, CD3E, CD3G, CD4, CD7, CD8A, CD8B, CD11A, CD11B, CD11C, CD11D, CD18, CD19, CD27, CD28, CD28 deleted for Lck binding (ICΔ), CD29, CD30, CD40, CD49A, CD49D, CD49F, CD69, CD79A, CD79B, CD84, CD96 (Tactile), CD103, CD160 (BY55), CD162 (SELPLG), CD226 (DNAM1), CD229 (Ly9), a ligand that specifically binds with CD83, CDS, CEACAM1, CRLF2, CRTAM, CSF2RA, CSF2RB, CSF3R, EPOR, Fc receptor gamma chain, Fc receptor ε chain, FCER1G, FCGR2C, FCGRA2, GADS, GHR, GITR, HVEM, IA4, ICAM-1, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA4, ITGA6, ITGAD, ITGAE, ITGAL, ITGAM, ITGAX, ITGB1, ITGB2, ITGB7, LAT, LEPR, LFA-1 (CD11a/CD18), LIGHT, LIFR, LMP1, LTBR, MPL, MYD88, NKG2C, NKP80 (KLRF1), OSMR, OX40, PD-1, PRLR, PSGL1, PAG/Cbp, SLAM (SLAMF1, CD150, IPO-3), SLAMF4 (C244, 2B4), SLAMF6 (NTB-A, Ly108), SLAMF7, SLAMF8 (BLAME), SLP-76, TILR2, TILR4, TILR7, TILR9, TNFR2, TNFRSF4, TNFRSF8, TNFRSF9, TNFRSF14, TNFRSF18, TRANCE/RANKL, VLA1, or VLA-6,or functional mutants and/or fragments thereof, or functional mutants and/or fragments thereof. In any of the embodiments disclosed herein, the lymphoproliferative element can include an extracellular ligand binding domain or a small molecule binding domain In some embodiments, the lymphoproliferative element can include a transmembrane domain In some embodiments, the transmembrane domain can include a transmembrane domain from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof.

In some embodiments of any aspect herein that include RIPs, the RIPs can comprise a binding polypeptide and a fusogenic element. In some embodiments, one or more viral envelope proteins comprise the binding polypeptide and the fusogenic element. In some embodiments, a viral envelope protein is a mutated viral envelope protein wherein a binding polypeptide of the viral envelope protein has been mutated to reduce/eliminate binding to a target cell (e.g. a T cell), but wherein such binding is provided by another (e.g. a heterologous) binding polypeptide which in further illustrative embodiments is also an activation element as provided herein (e.g. a polypeptide that binds CD3). In some embodiments, the viral envelope protein comprises the feline endogenous virus (RD114) envelope protein, an oncoretroviral amphotropic envelope protein, an oncoretroviral ecotropic envelope protein, the vesicular stomatitis virus envelope protein (VSV-G), the baboon retroviral envelope glycoprotein (BaEV), the murine leukemia envelope protein (MuLV), the influenza glycoprotein HA surface glycoprotein (HA), the influenza glycoprotein neurominidase (NA), the paramyxovirus Measles envelope protein H, the paramyxovirus Measles envelope protein F, the Tupaia paramyxovirus (TPMV) envelope protein H, the TPMV envelope protein F, the Nipah virus (NiV) envelope protein F, the NiV envelope protein G, the Sindbis virus (SINV) protein E1, the SINV protein E2, and/or functional variants or fragments of any of these envelope proteins. In some embodiments, the viral envelope protein is the NiV envelope protein G, wherein the NiV envelope protein G comprises one or more mutations in residues Y389, E501, W504, E505, V507, Q530, E533, or 1588 of SEQ ID NO:375. In some embodiments, Henipavirus-G protein is SEQ ID NO:375 with mutations E533A and/or Q530A. In some embodiments, one or more N- or O-glycosylation sites are mutated to improve pseudotyping and fusion. In some embodiments, one or more N-glycosylation sites are mutated for example, but not limited to, at one or more of N72, N159, N306, N378, N417, N481, or N529 of SEQ ID NO:375, or the corresponding glutamines of other Henipavirus-G proteins, to another amino acid such as glutamine. In some embodiments, one or more O-glycosylation sites are mutated from serine or threonine to another amino acid such as alanine. In some embodiments, one or more of the serine or threonine residues in the heavily O-glycosylated stalk domain from amino acids 103 to 137 of SEQ ID NO:375, is mutated to, for example, alanine. In other embodiments, the C-terminus of the Henipavirus-G protein can be modified and fused to a binding polypeptide and in illustrative embodiments, an activation element, such as an antibody or antibody mimetic, which in illustrative embodiments can be an anti-CD3 antibody or antibody mimetic.

In any of the aspects and embodiments provided herein that include a RIP, the RIP comprises a pseudotyping element on its surface that is capable of binding to a T cell and/or NK cell and facilitating membrane fusion of the RIP thereto. In some embodiments, the pseudotyping element is a viral envelope protein. In some embodiments, the viral envelope protein is one or more of the feline endogenous virus (RD114) envelope protein, the oncoretroviral amphotropic envelope protein, the oncoretroviral ecotropic envelope protein, the vesicular stomatitis virus envelope protein (VSV-G), the baboon retroviral envelope glycoprotein (BaEV), the murine leukemia envelope protein (MuLV), and/or the paramyxovirus Measles envelope proteins H and F, the Tupaia paramyxovirus (TPMV) envelope protein H, the TPMV envelope protein F, the Nipah virus (NiV) envelope protein F, the NiV envelope protein G, the Sindbis virus (SINV) protein E1, the SINV protein E2, or a fragment of any thereof that retains the ability to bind to resting T cells and/or resting NK cells. In illustrative embodiments, the pseudotyping element is VSV-G. As discussed elsewhere herein, the pseudotyping element can include a fusion with a T cell activation element, which in illustrative embodiments, can be a fusion with any of the envelope protein pseudotyping elements, for example MuLV or VSV-G, with an anti-CD3 antibody. In further illustrative embodiments, the pseudotyping elements include both a VSV-G and a fusion of an antiCD3scFv to MuLV.

In any of the aspects herein that include a RIP, the RIP(s) can comprise an activation element on their surface. In some embodiments, the activation element on the surface is a membrane-bound T cell activation element. In some embodiments, the activation element is a polypeptide capable of binding to a polypeptide on the surface of a lymphocyte, and in illustrative embodiments, a T cell and/or an NK cell. In some subembodiments of these and embodiments of any of the aspects provided herein,

In some embodiments, the T cell activation element comprises one or more of an antibody or an antibody mimetic capable of binding CD28, CD3, TCR α/β, CD28, or a mitogenic tetraspanin, or wherein said T cell activation element is a mitogenic tetraspanin. In some embodiments, the T cell activation element comprises the antibody or the antibody mimetic capable of binding CD3, and wherein the T cell activation element is bound to the membrane of the RIPs. In some embodiments, the membrane-bound anti-CD3 antibody or anti-CD3 antibody mimetic is an anti-CD3 scFv, an anti-CD3 scFvFc, or an anti-CD3 DARPin. In some embodiments, the anti-CD3 antibody or anti-CD3 antibody mimetic is bound to the membrane by a GPI anchor, such as a heterologous GPI anchor attachment sequence, wherein the anti-CD3 antibody or anti-CD3 antibody mimetic is a recombinant fusion protein with a MuLV viral envelope protein, with or without a mutation at a furin cleavage site, or wherein the anti-CD3 antibody or anti-CD3 antibody mimetic is a recombinant fusion protein with a VSV viral envelope protein, or wherein the anti-CD3 antibody or anti-CD3 antibody mimetic is a recombinant fusion protein with a Henipavirus-G envelope protein, or wherein the anti-CD3 antibody is a recombinant fusion protein with a NiV viral envelope protein. In some embodiments, the polypeptide capable of binding CD28 is CD80, or an extra-cellular domain thereof, bound to a CD16B GPI anchor attachment sequence.

In illustrative embodiments, the activation element is a T cell activation element capable of binding to a TCR complex polypeptide. In some embodiments, a TCR complex polypeptide is CD3D, CD3E, CD3G, CD3Z, TCRα, or TCRβ. In some embodiments, the activation element capable of binding to the TCR complex polypeptide is a polypeptide capable of binding to one or more of CD3D, CD3E, CD3G, CD3Z, TCRα, or TCRβ. In illustrative embodiments, the activation element activates ZAP-70. In some embodiments, the activation element includes a polypeptide capable of binding to CD16A, NKG2C, NKG2E, NKG2F, or NKG2H. In further embodiments, the polypeptide capable of binding to CD16A includes capable of binding to one or more of NKp46, 2B4, CD2, DNAM, NKG2C, NKG2D, NKG2E, NKG2F, or NKG2H. In some embodiments, the activation element is a polypeptide capable of binding to one or more of the following combinations: NKp46 and 2B4, NKp46 and CD2, NKp46 and DNAM, NKp46 and NKG2D, 2B4 and DNAM, or 2B4 and NKG2D. In some embodiments, the activation element can be two or more polypeptides capable of binding to polypeptides on the surface of a lymphocyte. In some embodiments, the activation element can be two or more polypeptides capable of binding to at least one of the following combinations: NKp46 and 2B4, NKp46 and CD2, NKp46 and DNAM, NKp46 and NKG2D, 2B4 and DNAM, or 2B4 and NKG2D.

In some embodiments, provided herein as separate aspects, or as components of, or as produced by, or used in, methods, uses, and compositions provided herein, are modified cells, in illustrative embodiments T cells, that have a dimmed surface polypeptide, or a population of any of the preceding modified cells that have a dimmed surface polypeptide. Such modified CD4+ cells or CD8+ cells or a population thereof can be CD3 dimmed, and can have the following characteristics (referred to herein as “Dimmed T Cell Characteristics”), and in illustrative embodiments have the following characteristics at the time of forming and/or administering the cell formulation:
i) at least 9%, 10% 15%, 20%, 30%, 40%, 50%, 60%, 70%, 80%, 90%, 95%, or 99%, or between 10% and 50%, 60%, 70%, 80%, 90%, 95%, or 99%, or between 50% and 60%, 70%, 80%, 90%, 95%, or 99%, or between 9%, 10% 15%, 20%, 30%, 40%, 50%, 60%, 70%, 80%, 90%, or 95% on the low end and 99% on the high end of the CD4+ cells in the cell formulation are surface CD3−;
ii) at least 18%, 20%, 30%, 40%, 50%, 60%, 70%, 80%, 90%, 95%, or 99%, or between 20% and 50%, 60%, 70%, 80%, 90%, 95%, or 99%, or between 50% and 60%, 70%, 80%, 90%, 95%, or 99%, or between 18%, 20%, 30%, 40%, 50%, 60%, 70%, 80%, 90%, or 95% on the low end and 99% on the high end of the CD8+ cells in the cell formulation are surface CD3−;
iii) within the population of cells that are either CD4+ or CD8+ in the cell formulation, at least 10.5%, 15%, 20%, 30%, 40%, 50%, 60%, 70%, 80%, 90%, 95%, or 99%, or between 10% and 50%, 60%, 70%, 80%, 90%, 95%, or 99%, or between 25% and 50%, 60%, 70%, 80%, 90%, 95%, or 99%, or between 50% and 60%, 70%, 80%, 90%, 95%, or 99%, or between 10.5%, 15%, 20%, 30%, 40%, 50%, 60%, 70%, 80%, 90%, or 95% on the low end and 99% on the high end, are surface CD3−;
iv) at least 1.5%, 2%, 3%, 4%, 5%, 6%, 7%, 8%, 9%, 10%, or 11%, or between 1.5% and 2%, 3%, 4%, 5%, 6%, 7%, 8%, 9%, 10%, or 11%, or between 5% and 6%, 7%, 8%, 9%, 10%, or 11%; or between 1.5%, 2%, 3%, 4%, 5%, 6%, 7%, 8%, 9% or 10% and 11% of the cells in the cell formulation, excluding RBCs, are surface CD3-CD4+;
v) at least 0.65%, 0.75%, 1%, 1.5%, 2%, 2.5%, 3%, 3.5%, 4%, 4.5% or 5%; between 0.65% and 0.75%, 1%, 1.5%, 2%, 2.5%, 3%, 3.5%, 4%, 4.5% or 5%; between 1% and 1.5%, 2%, 2.5%, 3%, 3.5%, 4%, 4.5% or 5%; or between 0.65%, 0.75%, 1%, 1.5%, 2%, 2.5%, 3%, 3.5%, 4%, or 4.5% on the low end and 5% on the high end of the cells in the cell formulation, excluding RBCs, are surface CD3-CD8+;
vi) less than 13%, 12%, 11%, 10%, 9%, 8%, 7%, 6%, 5%, 4%, 3%, 2%, 1%, 0.5%, or 0.1% of the cells in the cell formulation, excluding RBCs, are surface CD3+ and either CD4+ or CD8+ (the “Percent Total CD3+ cells”);
vii) within the population of cells that are either CD4+ or CD8+ in the cell formulation, less than 89%, 80%, 75%, 70%, 60%, 50%, 40%, 30%, 25%, 20%, 15%, 10%, 5%, or 1%, or between 89% and 50%, 40%, 30%, 20%, 10%, 5%, or 1%, or between 75% and 50%, 40%, 30%, 20%, 10%, 5%, or 1%, or between 50% and 40%, 30%, 20%, 10%, 5%, or 1%, or between 1% and 5%, 10%, 20%, 30%, 40%, 50%, 60%, 70%, 75% or 89%, are surface CD3+;
viii) CD4+ and/or CD8+ cells in the cell formulation have lower surface expression of CD3 than the surface expression of CD3 on CD4+ and/or CD8+ cells in blood collected from a healthy subject or a population of healthy subjects, wherein the surface expression of CD3 in the cell formulation is lower by at least a 10%, 20%, 25%, 30%, 40%, 50%, 60%, 70%, 80%, 90%, 95% or 99%, or between 10% and 20%, 25%, 30%, 40%, 50%, 60%, 70%, 80%, 90%, 95% or 99%, or between 50% and 60%, 70%, 80%, 90%, 95% or 99%, or between 10%, 20%, 25%, 30%, 40%, 50%, 60%, 70%, 80%, 90% or 95% on the low end and 99% on the high end; and/or
ix) the Percent Total CD3+ cells is reduced by at least 19%, 20% 30%, 40%, 50%, 60%, 70%, 80%, 90%, or 95% after contacting gene vectors, and in illustrative embodiments RIPs, compared to the Percent Total CD3+ cells in the absence of contacting the gene vectors, and in illustrative embodiments, RIPs.

In some embodiments, provided herein as separate aspects, or as components of, or as produced by, or used in, methods, uses, and compositions provided herein, are modified cells, in illustrative embodiments modified NK cells, that have a dimmed surface polypeptide, or a population of modified cells that have a dimmed surface polypeptide. Such modified cells, for example modified NK cells, or a population thereof can have one or more of CD16A, NKp46, 2B4, CD2, DNAM, NKG2C, NKG2D, NKG2E, NKG2F, or NKG2H dimmed, and can have the following characteristics (referred to herein as “Dimmed NK Cell Characteristics”), and in illustrative embodiments have the following characteristics at the time of forming and/or administering a cell formulation:
i) at least 9%, 10% 15%, 20%, 30%, 40%, 50%, 60%, 70%, 80%, 90%, 95%, or 99%, or between 10% and 50%, 60%, 70%, 80%, 90%, 95%, or 99%, or between 50% and 60%, 70%, 80%, 90%, 95%, or 99%, or between 9%, 10% 15%, 20%, 30%, 40%, 50%, 60%, 70%, 80%, 90%, or 95% on the low end and 99% on the high end of the CD56+ cells in the cell formulation are surface CD16A−, NKp46−, 2B4−, CD2−, DNAM−, NKG2C−, NKG2D−, NKG2E−, NKG2F−, and/or NKG2H−;
ii) within the population of cells that are CD56+ in the cell formulation, at least 10.5%, 15%, 20%, 30%, 40%, 50%, 60%, 70%, 80%, 90%, 95%, or 99%, or between 10% and 50%, 60%, 70%, 80%, 90%, 95%, or 99%, or between 25% and 50%, 60%, 70%, 80%, 90%, 95%, or 99%, or between 50% and 60%, 70%, 80%, 90%, 95%, or 99%, or between 10.5%, 15%, 20%, 30%, 40%, 50%, 60%, 70%, 80%, 90%, or 95% on the low end and 99% on the high end, are surface CD16A−, NKp46−, 2B4−, CD2−, DNAM−, NKG2C−, NKG2D−, NKG2E−, NKG2F−, and/or NKG2H−;
iii) at least 1.5%, 2%, 3%, 4%, 5%, 6%, 7%, 8%, 9%, 10%, or 11%, or between 1.5% and 2%, 3%, 4%, 5%, 6%, 7%, 8%, 9%, 10%, or 11%, or between 5% and 6%, 7%, 8%, 9%, 10%, or 11%; or between 1.5%, 2%, 3%, 4%, 5%, 6%, 7%, 8%, 9% or 10% and 11% of the cells in the cell formulation, excluding RBCs, are CD56+ and surface CD16A−, NKp46−, 2B4−, CD2−, DNAM−, NKG2C−, NKG2D−, NKG2E−, NKG2F−, and/or NKG2H−;
iv) less than 13%, 12%, 11%, 10%, 9%, 8%, 7%, 6%, 5%, 4%, 3%, 2%, 1%, 0.5%, or 0.1% of the cells in the cell formulation, excluding RBCs, are surface CD16A+, NKp46+, 2B4+, CD2+, DNAM+, NKG2C+, NKG2D+, NKG2E+, NKG2F+, and/or NKG2H+and CD56+ (the “Percent Total CD16A, NKp46, 2B4, CD2, DNAM, NKG2C, NKG2D, NKG2E, NKG2F, and/or NKG2H cells”);
v) within the population of cells that are CD56+ in the cell formulation, less than 89%, 80%, 75%, 70%, 60%, 50%, 40%, 30%, 25%, 20%, 15%, 10%, 5%, or 1%, or between 89% and 50%, 40%, 30%, 20%, 10%, 5%, or 1%, or between 75% and 50%, 40%, 30%, 20%, 10%, 5%, or 1%, or between 50% and 40%, 30%, 20%, 10%, 5%, or 1%, or between 1% and 5%, 10%, 20%, 30%, 40%, 50%, 60%, 70%, 75% or 89%, are surface CD16A+, NKp46+, 2B4+, CD2+, DNAM+, NKG2C+, NKG2D+, NKG2E+, NKG2F+, and/or NKG2H+and CD56+;
vi) CD56+ cells in the cell formulation have lower surface expression of CD16A, NKp46, 2B4, CD2, DNAM, NKG2C, NKG2D, NKG2E, NKG2F, and/or NKG2H than the surface expression of CD16A, NKp46, 2B4, CD2, DNAM, NKG2C, NKG2D, NKG2E, NKG2F, and/or NKG2H, respectively, on CD56+ cells in blood collected from a healthy subject or a population of healthy subjects, wherein the surface expression of CD16A, NKp46, 2B4, CD2, DNAM, NKG2C, NKG2D, NKG2E, NKG2F, and/or NKG2H in the cell formulation is lower by at least a 10%, 20%, 25%, 30%, 40%, 50%, 60%, 70%, 80%, 90%, 95% or 99%, or between 10% and 20%, 25%, 30%, 40%, 50%, 60%, 70%, 80%, 90%, 95% or 99%, or between 50% and 60%, 70%, 80%, 90%, 95% or 99%, or between 10%, 20%, 25%, 30%, 40%, 50%, 60%, 70%, 80%, 90% or 95% on the low end and 99% on the high end; and/or
vii) the Percent Total CD16A, NKp46, 2B4, CD2, DNAM, NKG2C, NKG2D, NKG2E, NKG2F, and/or NKG2H cells is reduced by at least 19%, 20% 30%, 40%, 50%, 60%, 70%, 80%, 90%, or 95% after contacting gene vectors, and in illustrative embodiments RIPS, compared to the Percent Total CD16A, NKp46, 2B4, CD2, DNAM, NKG2C, NKG2D, NKG2E, NKG2F, and/or NKG2H cells in the absence of contacting the gene vectors, and in illustrative embodiments, RIPs.

In any of the aspects herein, in illustrative embodiments that include the Dimmed T Cell Characteristics or the Dimmed NK Cell Characteristics, the modified cells can be recently activated within the prior 7, 6, 5, 4, 3, 2, or 1 days.

In some embodiments of any of the cell formulation aspects or embodiments herein, or any method or use aspect that includes a cell formulation, or any of the population embodiments, some of the modified CD4+, modified CD8+, modified CD56+, modified T cells and/or modified NK cells therein, are in cell aggregates. In some embodiments, at least 1%, 2%, 3%, 4%, 5%, 7.5%, 10%, 15%, 20%, or 25%, or between 1% and 10% 15%, 20%, 25%, 50%, and 75% of the white blood cells, modified CD4+ cells, modified CD8+ cells, modified CD56+ cells, modified T cells and/or modified NK cells in the cell formulation are in cell aggregates. In some embodiments, the cell aggregates are greater than 15, 25, 30, 35, 40, 50, or 60 μm in diameter, or between 25 and 50, 60, 75, 100, 125, 150, 200 or 250 μm in diameter. In some embodiments, modified cells are in aggregates comprising at least 4, 5, 6, 7, 8, 9, 10, 20, 30, 40, 50, 100, 250, 500, 1,000, 2,500, 5,000, or 10,000 white blood cells or 5 to 500, 5 to 250, 5 to 100, 10 to 500, 10 to 250, or 10 to 100 white blood cells. Furthermore, in some embodiments, including sub-embodiments of the immediately preceding embodiment, at least 1%, 2%, 3%, 4%, 5%, 7.5%, 10%, 15%, 20%, or 25%, or between 1% and 10% 15%, 20%, 25%, 50%, and 75% of white blood cells, the modified T and/or NK cells in the cell formulation are in aggregates comprising, or comprising at least 4, 5, 6, 8, or 5 to 500, 5 to 250, 5 to 100, 10 to 500, 10 to 250, or 10 to 100 white blood cells, modified T cells and/or NK cells. Further, in some embodiments, the cell formulation comprises aggregates of modified T cells and/or NK cells, in some embodiments along with unmodified T cells and/or NK cells and/or other white blood cells, capable of being retained by a coarse filter having a pore diameter of at least 15, 20, 25, 30, 40, 50, or 60 μm. In certain illustrative embodiments, at least 5% of the white blood cells, T cells, NK cells, modified T cells and/or modified NK cells are in cell aggregates. In certain sub-embodiments, the cell aggregates are greater than 40 μm in diameter and/or are capable of being retained by a course filter having a pore diameter of at least 40 μm. In some sub-embodiments, the cell aggregates comprise 5 to 500 white blood cells or modified T cells.

In some embodiments of any of the aspects or embodiments herein that include administering cells, populations, or cell formulations to a subject, a persisting population of genetically modified cells, and in illustrative embodiments genetically modified T cells and/or NK cells, or a population of progeny cells or genetically modified progeny cells derived from the modified cells, persists in the subject for at least 1, 2, 3, 4, 5, 6, 7, 14, 17, 21, or 28 days or 1, 2, or 1, 2, 3, 4, 5, 6, 7, 8, 9, 10, 11, or 12 months or 1, 2, 3, 4, or 5 years after administration. In some embodiments, at least 50%, 60%, 70%, 80%, 90% or 95% of the genetically modified cells, and in illustrative embodiments CAR-T cells, express a first polypeptide comprising the transgene, and in illustrative embodiments express the engineered T cell receptor, or the CAR. In some embodiments, at least 50%, 60%, 70%, 80%, 90% or 95% of the genetically modified cells expressing the first polypeptide comprising the transgene are circulating in the blood and/or at the site of a tumor, for example a solid tumor and the remainder of the genetically modified cells are subcutaneous. In some embodiments, the persisting population is subcutaneous, is circulating in the blood, and/or is at the site of a tumor, for example a solid tumor. In some embodiments, the subcutaneous region contains no artificial matrix components.

In some embodiments, the persisting population is detectable by histology. In some embodiments, the persisting population persists subcutaneously for at least or up to 14, 21, 28, 50, 60, 90 days and is detectable by histology. In some embodiments, the persisting population is detectable by FACS, for example FACs for the CAR or a removal tag (e.g. eTag), for example as 2 genetically modified cells/μl blood, or by qPCR, for example qPCR or sequencing of the transgene or across a chimeric junction of a CAR, or for a non-human subject treated with human engineered cells, such as human CAR-T cells, human RNAse P (hRNAseP). In some embodiments, the persisting population is detectable in the blood.

In some embodiments of any of the aspects provided herein, including but not limited to the method and use aspects provided hereinabove in this Exemplary Embodiments section, the modified cells, for example modified T cells and/or NK cells, or a population thereof, have a surface polypeptide dimmed, which in illustrative embodiments can be a TCR complex polypeptide, and in illustrative sub-embodiments, CD3. Such dimmed cells, including populations thereof, in illustrative embodiments exhibit any of the Dimmed T Cell Characteristics and/or Dimmed NK Cell Characteristics provided herein.

In some embodiments of any of the aspects provided herein, including but not limited to the method and use aspects provided hereinabove in this Exemplary Embodiments section, some (e.g. at least 5%, 7.5%, or 10%) of the modified cells, for example modified T cells and/or NK cells, or a population thereof, or a population thereof, are in aggregates, as disclosed herein.

In some embodiments of any of the aspects provided herein, including but not limited to the method and use aspects provided hereinabove in this Exemplary Embodiments section the cells form a population, which can be a persistent population, as disclosed herein.

In any of the persisting population or population of progeny cells aspects or embodiments herein, or any aspect or embodiment herein that includes a persisting population or a population of progeny cells, the number of modified cells, such as modified T cells and/or NK cells, and in illustrative embodiments genetically modified T cells and/or NK cells, comprises at least 100, 1×10³, 1×10⁴, 1×10⁵, 1×10⁶, 1×10⁷, 1×10⁸, 1×10⁹, 1×10¹⁰, 1×10¹¹, or 1×10¹² cells or between 1×10³ and 1×10⁴, 1×10⁵, 1×10⁶, 1×10⁷, 1×10⁸, or 1×10⁹ cells. In some embodiments, the modified cells, and in illustrative embodiments modified T cells and/or NK cells present in the cell formulation administered to a subject multiply at least 5, 10, 15, 20, 25, 50, 75, 100, 250, 500, 750, 1,000, 2,500, 5,000, or 10,000 fold in the subject, for example to form a persisting population or a population of progeny cells.

In some embodiments, the persisting population or the population of progeny cells express an engineered T cell receptor or a CAR, and the persisting population or the population of progeny cells is detected indirectly by a durable clinical response. For example, such persistence can be detected by detecting stable disease, a partial response, or a complete response with a duration of response of at least 3, 6, 9, 12, 18, or 24 months after initial observation of a clinical response, which in some embodiments is stable disease for patients experiencing progressive disease before administration of the cells, and in illustrative embodiments administration of the engineered T cell or CAR-T therapy.

In any of the aspects provided herein that include a step of collecting blood, the volume of blood collected can be for example, between 5 ml and 600 ml. More volumes and ranges are provided elsewhere in this specification, and in some embodiments, include the Small Volume Elements provided herein. In some embodiments when collected blood is processed using a filter, in illustrative embodiments a leukoreduction filter, the volume of blood sample applied to a filter is 600, 500, 400, 300, 200, 150, 120, 100, 75, 50, 40, 30, 25, 20, 15, 10, or 5 ml or less. In illustrative embodiments, the volume of blood sample applied to a filter is 50, 25, 20, 15, 10, 9, 8, 7, 6, 5, 4, 3, 2, or 1 ml or less.

In some embodiments of any of the aspects provided herein, the cell formulation, which in illustrative embodiments can be in a syringe, has a volume of between 0.5 ml and 20 ml, 15 ml, 10 ml, 5 ml, or 1 ml; or between 1 ml and 20 ml, 15 ml, 10 ml, 5 ml, 4 ml, 3 ml, 2 ml or less; or between 2 ml and 20 ml, 15 ml, 10 ml, 7 ml or 5 ml; or between 5 ml and 20 ml, 15 ml or 10 ml, or between 3 ml and 12 ml, or less than 3 ml. In some embodiments of any of the aspects or embodiments provided herein, wherein blood is collected from a subject, the blood collected has a volume of between 2.5 ml and 75 ml, 60 ml, 50 ml, 40 ml, 30 ml, 25 ml, 20 ml, 15 ml, 10 ml, or 5 ml, or between 5 ml and 75 ml, 60 ml, 50 ml, 40 ml, 30 ml, 25 ml, 20 ml, 15 ml, or 10 ml, or between 10 ml and 75 ml, 60 ml, 50 ml, 50 ml, 40 ml, 30 ml, 25 ml, or 20 ml, or between 15 ml and 75 ml, 60 ml, 50 ml, 50 ml, 40 ml, 30 ml, 25 ml, or 20 ml, or between 20 ml and 75 ml, 60 ml, 50 ml, 40 ml, 30 ml, or 25 ml, or between 25 ml and 75 ml, 70 ml, 60 ml, 50 ml, 40 ml, and 30 ml, or between 5 ml, 10 ml, or 15 ml on the low end and 20 ml on the high end. In some embodiments when collected blood is processed using a filter, in illustrative embodiments a leukoreduction filter, the volume of a whole blood sample, or a fraction thereof applied to a filter can be between 2.5 ml and 75, 50, 40, 30, 25, 20, 15, or 10. In illustrative embodiments, the volume of a whole blood sample, or a fraction thereof applied to a filter is between 10 ml and 50, 25, 20, 15 ml. In some embodiments, the volume of a whole blood sample, or fraction thereof applied to a filter is 10, 9, 8, 7, 6, 5, 4, 3, 2, or 1 ml or less. In some embodiments of any of the aspects or embodiments provided herein, the volume of the reaction mixture is between 2.5 ml and 75 ml, 60 ml, 50 ml, 40 ml, 30 ml, 25 ml, 20 ml, 15 ml, 10 ml, or 5 ml, or between 5 ml and 75 ml, 70 ml, 60 ml, 50 ml, 40 ml, 30 ml, 25 ml, 20 ml or 10 ml, or between 10 ml and 75 ml, 70 ml, 60 ml, 50 ml, 40 ml, 30 ml, 25 ml, 20 ml, or 15 ml, or between 15 ml and 75 ml, 70 ml, 60 ml, 50 ml, 40 ml, 30 ml, 25 ml, or 20 ml, or between 20 ml and 75 ml, 70 ml, 60 ml, 50 ml, 40 ml, 30 ml, or 25 ml or between 25 ml and 75 ml, 70 ml, 60 ml, 50 ml, 40 ml, and 30 ml. The volumes for the cell formulation, the collected blood, and the reaction mixture in this paragraph are herein referred to as the “Small Volume Elements.” In illustrative subembodiments of embodiments that include the Small Volume Elements, the cell formulation is adapted for subcutaneous delivery, wherein the number of modified cells, such as modified T cells and/or NK cells in the modified cell formulation, is between 1.5×10⁴ and 1.5×10⁹, 1×10⁹, 1×10⁸, or 1×10⁷ or between 1×10⁵ and 1.5×10⁸, between 1 ×10⁵ and 1×10⁷, or between 1×10⁶ and 1×10⁸, or between 2×10⁶ and 1×10⁸, or in illustrative embodiments, between 3×10⁴ and 3×10⁷, between 1×10⁵ and 3×10⁷, or between 1×10⁶ and 3×10⁷ modified T cells, NK cells, CD4+ cells, CD8+ cells, and/or CD56+ cells.

In some embodiments, a contacting step is performed in a blood processing bag or other incubation bag, for example wherein whole blood, or a fraction thereof is added to an incubation bag comprising RIPs to form a reaction mixture or wherein the RIPs are added to the incubation bag comprising the whole blood to form the reaction mixture.

In illustrative embodiments of any of the encapsulated gene vectors (e.g. gene vector particles), and in illustrative embodiments retroviral particle, aspects provided herein, or any other aspect that includes a gene vector particle, the gene vector particle is substantially free of the protein transcript encoded by the nucleic acid of the gene vector particle, for example substantially free an engineered T cell receptor or CAR encoded by the nucleic acid of the gene vector gene vector particle.

In some embodiments, a sample such as a blood sample or a reaction mixture before, during, or after an incubation, is applied to a leukoreduction filter for example to remove RIPs not associated with lymphocytes. In some embodiments, at least 75%, 80%, 85%, 86%, 87%, 88%, 89%, 90%, 91%, 92%, 93%, 94%, 95%, 96%, 97%, 98%, 99%, 99.5%, or 99.9% of the RIPs not associated with the lymphocytes are removed from a reaction mixture. In some embodiments, a reaction mixture is filtered over the leukoreduction filter at a flow rate of between 0.25 and 1, 1.5, 2, 2.5, 3, 3.5, 4, 4.5, or 5 ml/min, or between 0.5 and 2 ml/min. In some embodiments, the reaction mixture is filtered over the leukoreduction filter using a syringe. In some embodiments, the syringe is at less than an 80°, 75°, 70°, 65°, 60°, 55°, 50°, or 45° angle with respect to a channel (e.g. tubing) that is in fluid communication with the leukoreduction filter, when the reaction mixture is filtered. In some embodiments, blood cells and modified lymphocytes are not moved across a junction with a greater than 70°, 75°, or 80° angle during the removing the RIPs. In some embodiments, the leukoreduction filter has an effective filtration area of between 3 cm² and 5 cm² and in these or other embodiments, the diameter of the pore in the filter is between 2 and 6 μm. In some embodiments, cells retained by the leukoreduction filter after a reaction mixture is filtered over the leukoreduction filter, are washed with a volume of wash buffer that is 0.25 to 3 times the volume of the reaction mixture. In some embodiments, the removing of the RIPs is performed within a filter assembly comprising a syringe, the leukoreduction filter in fluid communication with the syringe, and one or more bags in fluid communication with the leukoreduction filter. In some embodiments, the removing of the RIPs is performed within a filter assembly comprising a second syringe and a second bag, wherein the second bag is in fluid communication with the leukoreduction filter.

In any aspect provided herein that includes a polynucleotide(s), such as an isolated polynucleotide(s) encoding a CAR and/or an LE, such polynucleotides or isolated polynucleotides can be contained in one or more containers, and for example in 0.1 ml to 10 ml of a solution. Such polynucleotides can contain substantially-pure, GMP grade, recombinant vectors (e.g., replication incompetent retroviral particles). In some embodiments, such polynucleotides comprise recombinant naked DNA vectors. In illustrative embodiments, such polynucleotides are a container of replication incompetent retroviral particles having between 1×10⁶ and 5×10⁹, 1×10⁷ and 1×10⁹, 5×10⁶ and 1×10⁸, 1×10⁶ and 5×10⁷, 1×10⁶ and 5×10⁶ or between 5×10⁷ and 1×10⁸ retroviral Transducing Units (TUs) or TUs/ml, or at least 100, 1,000, 2,000 or 2,500 TUs/ng p24.

In some embodiments when a leukoreduction filter is used to fraction collected blood, the pore diameter of the filter is less than 10, 7.5, 5, 4, or 3 μm or from 0.5 to 4 μm, or from 2 μm to 6 μm. In some embodiments, the leukoreduction filter assembly can collect and/or retain at least 90%, 95%, 96%, 97%, 98%, 99%, 99.9% or 99.99% of the white blood cells in the blood sample. In illustrative embodiments, the leukoreduction filter assembly can collect at 99%, 99.9% or 99.99% of the white blood cells in the blood sample. In some embodiments, at least 75%, 80%, 85%, 90%, or 95%, or between 75% and 99.99%, 80% and 99.99%, 85% and 99.99%, 90% and 99.99%, or 95% and 99.99% of the non-leukocyte cells pass through the filter and are not collected.

In any of the aspects provided herein, the contacting step including with an optional incubation combined can be performed (or can occur) for 14, 12, or 10 hours or less, or in illustrative embodiments for 8, 6, 4, 3, 2, or 1 hour or less, or in certain further illustrative embodiments less than 8 hours, less than 6 hours, less than 4 hours, 2 hours, less than 1 hour, less than 30 minutes or less than 15 minutes, but in each case there is at least an initial contacting step in which retroviral particles and cells are brought into contact in suspension in a transduction reaction mixture. In other embodiments, the reaction mixture can be incubated for between 15 minutes and 12 hours, 15 minutes and 10 hours, 15 minutes and 8 hours, 15 minutes and 6 hours, 15 minutes and 4 hours, 15 minutes and 2 hours, 15 minutes and 1 hour, 15 minutes and 45 minutes, or 15 minutes and 30 minutes. In other embodiments, the reaction mixture can be incubated for between 30 minutes and 12 hours, 30 minutes and 10 hours, 30 minutes and 8 hours, 30 minutes and 6 hours, 30 minutes and 4 hours, 30 minutes and 2 hours, 30 minutes and 1 hour, or 30 minutes and 45 minutes. In other embodiments, the reaction mixture can be incubated for between 1 hour and 12 hours, 1 hour and 8 hours, 1 hour and 4 hours, or lhour and 2 hours. In another illustrative embodiment, the contacting is performed for between an initial contacting step only (without any further incubating in the reaction mixture including the retroviral particles free in suspension and cells in suspension) without any further incubation in the reaction mixture, or a 5 minute, 10 minute, 15 minute, 30 minute, or 1 hour incubation in the reaction mixture. In certain embodiments, the contacting can be performed (or can occur) for between 30 seconds or 1, 2, 5, 10, 15, 30 or 45 minutes, or 1, 2, 3, 4, 5, 6, 7, or 8 hours on the low end of the range, and between 10 minutes, 15 minutes, 30 minutes, or 1, 2, 4, 6, 8, 10, 12, 18, 24, 36, 48, and 72 hours on the high end of the range. In illustrative embodiments, the contacting can be performed (or can occur) for between a contacting only, 30 seconds or 1, 2, 5, 10, 15, 30 or 45 minutes, or 1 hour on the low end of the range, and between 2, 4, 6, and 8 hours on the high end of the range. In some embodiments, the RIPs can be immediately washed out after adding them to the cell(s) to be modified, genetically modified, and/or transduced such that the contacting time is carried out for the length of time it takes to wash out the RIPs. Accordingly, typically the contacting includes at least an in initial contacting step in which a retroviral particle(s) and a cell(s) are brought into contact in suspension in a transduction reaction mixture. Such methods can be performed without prior activation.

In illustrative embodiments of methods provided herein, the contacting step with optional incubating, is performed at temperatures between 32° C. and 42° C., such as at 37° C. as provided in more detail herein. In other illustrative embodiments, the contacting step with optional incubating, is performed at temperatures lower than 37° C., such as between 1° C. and 25° C., 2° C. and 20° C., 2° C. and 15° C., 2° C. and 6° C., or 3° C. and 6° C. The optional incubating associated with the contacting step at these temperatures can be performed for any length of time discussed herein. In illustrative embodiments, the optional incubating associated with these temperatures is performed for 1 hour or less, such as for 0 to 55 minutes (i.e., 55 minutes or less), 0 to 45 minutes (i.e., 45 minutes or less), 0 to 30 min (i.e., 30 minutes or less), 0 to 15 minutes (i.e., 15 minutes or less), 0 to 10 minutes (i.e., 10 minutes or less), 0 to 5 minutes (i.e., 5 minutes or less), 5 to 30 minutes, 5 to 15 minutes, or 10 to 30 minutes. In further illustrative embodiments, the cold contacting and incubating is performed at temperatures between 2° C. and 15° C. for between 0 to 55 minutes, 0 to 45 minutes, 0 to 30 min, 0 to 15 minutes, 0 to 10 minutes, 0 to 5 minutes, 5 to 15 minutes, or 10 to 30 minutes. In other further illustrative embodiments, the cold contacting and incubating is performed for 5 to 30 minutes at a temperature between 1° C. and 25° C., 2° C. and 20° C., 2° C. and 15° C., 2° C. and 6° C., or 3° C. and 6° C.

In certain embodiments that comprise a contacting step at the colder temperatures provided immediately above, a secondary incubation is typically performed by suspending cells after an optional wash step in a solution comprising recombinant vectors, in illustrative embodiments retroviral particles. In illustrative embodiments, the secondary incubation is performed at temperatures between 32° C. and 42° C., such as at 37° C. The optional secondary incubation can be performed for any length of time discussed herein. In illustrative embodiments, the optional secondary incubation is performed for 6 hours or less, such as for 1 to 6 hours, 1 to 5 hours, 1 to 4 hours, 1 to 3 hours, 1 to 2 hours, 2 to 4 hours, 30 minutes to 4 hours, 10 minutes to 4 hours, 5 minutes to 4 hours, 5 minutes to 1 hour, 1 minute to 5 minutes, or less than 5 minutes. Thus, in some illustrative embodiments, optionally the T cell and/or NK cell activation element is on the surface of the RIPs, the contacting is performed at between 2° C. and 15 ° C., and optionally between 2° C. and 6° C., for less than 1 hour, optionally after which the TNCs are incubated at between 32° C. and 42° C. for between 5 minutes and 8 hours, or in illustrative embodiments, between 5 minutes and 4 hours, and optionally after which the modified T cells and/or NK cells are collected on a filter to form the cell formulation

In some embodiments, no more than 16 hours, 14 hours, 12 hours, 8 hours, 4 hours, 2 hours, or 1 hour pass, or between 5, 10, 15, 30, 45, or 60 minutes on the low end of the range, and between 1.5, 2, 4, 6, 8, 10, 12, 14, and 16 hours on the high end of the range, for example between 5 minutes and 16 hours, 5 minutes and 12 hours, 5 minutes and 8 hours, 5 minutes and 6 hours, 5 minutes and 4 hours, 5 minutes and 3 hours, 5 minutes and 2 hours, or 5 minutes and 1 hour pass, between the time blood, TNCs, or PBMCs are contacted with recombinant nucleic acid vectors, which in illustrative embodiments are replication incompetent retroviral particles, and the time the modified cells are suspended and thus formulated in a delivery solution to form a cell formulation. In some embodiments, the time between when the cells are contacted with the replication incompetent retroviral particles and when the modified cells are formulated in a delivery solution can be between 1 and 16 hours, 1 and 14 hours, 1 and 12 hours, 1 and 8 hours, 1 and 6 hours, 1 and 4 hours, or 1 and 2 hours. In some embodiments, no more than 16 hours, 14 hours, 12 hours, 8 hours, 4 hours, 2 hours, or 1 hours pass between the time blood is collected from the subject and the time the modified lymphocytes are reintroduced into the subject. In some embodiments, the time between when the blood is collected from the subject and when the modified lymphocytes are reintroduced into the subject can be between 1 and 16 hours, 1 and 14 hours, 1 and 12 hours, 1 and 8 hours, 1 and 6 hours, 1 and 4 hours, or 1 and 2 hours.

In any of the aspects provided herein that include an administering step, in illustrative embodiments, administered cells are processed ex vivo, for example using any of the methods comprising contacting and formulating steps provided herein, for less than 24, 18, 12, 10, 8, 6, 4, 2, or 1 hour or 30 or 15 minutes, or for between 15 minutes and 24, 18, 12, 10, 8, 6, 4, 2, 1, or 0.5 hours, or for between 1 hour and 24, 18, 12, 10, 8, 6, 4, or 2 hours, before the administration. Thus, in certain embodiments such ex vivo times can be the time between collecting blood from a subject and intravenous, intramuscular, intratumor, intraperitoneal, and in illustrative embodiments subcutaneous administration of modified lymphocytes, in illustrative embodiments derived from lymphocytes from the subject, to the subject

In some embodiments of any relevant aspect herein, some or all of the T and NK cells do not yet express the recombinant nucleic acid or have not yet integrated the recombinant nucleic acid into the genome of the cell before being used or included in any of the methods or compositions provided herein, including, but not limited to, being introduced or reintroduced back into a subject, or before, or at the time of being used to prepare a cell formulation. In some embodiments, at least 25%, 30%, 40%, 45%, 50%, 55%, 60%, 65%, 70%, 75%, 80%, 85%, 90%, 95%, 96%, 97%, 98%, 99%, or all of the modified T and NK cells do not express a CAR or transposase, and/or do not have a CAR associated with their cell membrane, when the modified lymphocytes are introduced or reintroduced back into a subject, and in illustrative embodiments introduced or reintroduced back into a subject subcutaneously or intramuscularly, or when used to prepare a cell formulation. In other embodiments, provided herein are cell formulations wherein at least 25%, 30%, 40%, 45%, 50%, 55%, 60%, 65%, 70%, 75%, 80%, 85%, 90%, 95%, 96%, 97%, 98%, 99%, or all of the modified T and/or NK cells in a cell formulation contain recombinant viral reverse transcriptase and/or integrase. In illustrative embodiments, at least 25%, 50%, 60%, 70%, 75%, 80%, 85%, 90%, 95%, 96%, 97%, 98%, 99%, or all of the modified T and NK cells do not express a CAR, and/or do not have a CAR associated with their cell membrane when the modified lymphocytes are introduced or reintroduced back into a subject, and in illustrative embodiments introduced or reintroduced back into a subject subcutaneously or intramuscularly, or when used to prepare a cell formulation. In illustrative embodiments, at least 25%, 50%, 55%, 60%, 65%, 70%, 75%, 80%, 85%, 90%, 95%, 96%, 97%, 98%, 99%, or all of the modified T and NK cells do not express a recombinant mRNA (e.g., encoding a CAR) when the lymphocytes are introduced or reintroduced into a subject, and in illustrative embodiments introduced or reintroduced back into a subject subcutaneously or intramuscularly, or when used to prepare a cell formulation. In some embodiments, greater than 50%, 60%, 70%, 75%, 80% or 90% of the cells, NK cells, and/or T cells in a cell formulation are viable.

In some embodiments, at least 20%, 25%, 30%, 35%, 40%, 45%, 50%, 55%, 60%, 65%, 70%, 75%, 80%, 85%, 90%, 95%, 96%, 97%, 98%, 99%, or all of the modified T and NK cells do not have the recombinant nucleic acid stably integrated into their genomes when the lymphocytes are introduced or reintroduced into a subject, and in illustrative embodiments introduced or reintroduced back into a subject subcutaneously or intramuscularly, or when used to prepare a cell formulation. In illustrative embodiments, at least 25%, 30%, 40%, 45%, 50%, 55%, 60%, 65%, 70%, 75%, 80%, 85%, 90%, 95%, 96%, 97%, 98%, 99%, or all of the modified T and NK cells do not have the recombinant nucleic acid stably integrated into their genomes when the lymphocytes are introduced or reintroduced into a subject, and in illustrative embodiments introduced or reintroduced back into a subject subcutaneously or intramuscularly, or when used to prepare a cell formulation. In some embodiments of any of the aspects herein that include modified, genetically modified, transduced, and/or stably transfected lymphocytes, any percentage of the lymphocytes can be modified, genetically modified, transduced, and/or stably transfected when the lymphocytes are introduced or reintroduced back into a subject, and in illustrative embodiments introduced or reintroduced back into a subject subcutaneously or intramuscularly, or when a cell formulation is prepared. In some embodiments, at least 4%, 5%, 10%, 15%, 20%, 25%, 30%, 35%, 40%, 45%, 50%, 55%, 60%, 65%, 70%, 75%, 80%, 85%, 90%, or 95% of the lymphocytes are modified. In illustrative embodiments, between 4%, 5%, 10%, 15%, 20%, 25%, 30%, 35%, 40%, 45%, 50%, 55%, 60%, 65%, and 70% of the lymphocytes are modified on the low end of the range and 10%, 15%, 20%, 25%, 30%, 35%, 40%, 45%, 50%, 55%, 60%, 65%, 70%, 75%, 80%, 85%, 90%, and 95% of the lymphocytes are modified on the high end of the range. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified lymphocytes are not genetically modified, transduced, or stably transfected. In illustrative embodiments, at least 25%, 30%, 35%, 40%, 45%, 50%, 55%, 60%, 65%, 70%, 75%, 80%, 85%, 90%, 95%, 96%, 97%, 98%, 99%, or all of the modified lymphocytes are not genetically modified, transduced, or stably transfected. In some embodiments, between 5%, 10%, 15%, 20%, 25%, 30%, 35%, 40%, 45%, 50%, 55%, 60%, 65%, and 70% of the modified lymphocytes are not genetically modified, transduced, or stably transfected on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified lymphocytes are not genetically modified, transduced, or stably transfected on the high end of the range (e.g., between 10% and 95%). Genetically modified lymphocytes containing a recombinant nucleic acid can either have the recombinant nucleic acid extrachromosomal or integrated into the genome. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the genetically modified lymphocytes have an extrachromosomal recombinant nucleic acid. In illustrative embodiments, at least 25%, 30%, 35%, 40%, 45%, 50%, 55%, 60%, 65%, 70%, 75%, 80%, 85%, 90%, 95%, 96%, 97%, 98%, 99%, or all of the genetically modified lymphocytes have an extrachromosomal recombinant nucleic acid. In some embodiments, between 5%, 10%, 15%, 20%, 25%, 30%, 35%, 40%, 45%, 50%, 55%, 60%, 65%, and 70% of the modified or genetically modified lymphocytes have an extrachromosomal recombinant nucleic acid on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes have an extrachromosomal recombinant nucleic acid on the high end of the range (e.g. between 10% and 95%). In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified or genetically modified lymphocytes are not transduced or stably transfected. In illustrative embodiments, at least 25%, 30%, 35%, 40%, 45%, 50%, 55%, 60%, 65%, 70%, 75%, 80%, 85%, 90%, 95%, 96%, 97%, 98%, 99%, or all of the modified or genetically modified lymphocytes are not transduced or stably transfected. In some embodiments, between 5%, 10%, 15%, 20%, 25%, 30%, 35%, 40%, 45%, 50%, 55%, 60%, 65%, and 70% of the modified or genetically modified lymphocytes are transduced or stably transfected on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes are not transduced or stably transfected on the high end of the range.

In certain embodiments disclosed herein including subcutaneous or intramuscular delivery of a cell formulation, the cells are formulated in a manner that is compatible with, effective for, and/or adapted for subcutaneous or intramuscular delivery such that fewer of the modified or genetically modified lymphocytes can engraft if delivered intravenously compared to when delivered subcutaneously. In some embodiments, at least 5%, 10%, 15%, 20%, 25%, 30%, 35%, 40%, 45%, 50%, 55%, 60%, 65%, 70%, 75%, 80%, 85%, 90%, or 95% fewer lymphocytes engraft when delivered intravenously compared to when delivered subcutaneously or intramuscularly. In some embodiments, the solution comprises at least two of unmodified lymphocytes, modified lymphocytes, and genetically modified lymphocytes. In some embodiments, the solution comprises more unmodified lymphocytes than modified lymphocytes. In some embodiments, the percent of T cells and NK cells that are modified, genetically modified, transduced, and/or stably transfected is at least 5%, at least 10%, at least 15%, or at least 20%. As illustrated in the Examples herein, in exemplary methods provided herein for transducing lymphocytes in whole blood, between 1% and 20%, or between 1% and 15%, or between 5% and 15%, or between 7% and 12% or about 10% of lymphocytes are genetically modified and/or transduced. In some embodiments, the lymphocytes are not contacted with a recombinant nucleic acid vector, such as a RIP, and are not modified. In illustrative embodiments, the lymphocytes are tumor infiltrating lymphocytes.

In some embodiments of any aspect herein wherein a formulation is administered to a subject, a second formulation is administered to the subject at a second, third, fourth, etc. timepoint between 1 day and 1 month, 2 months, 3 months, 6 months, or 12 months after the administering a first cell formulation, wherein the second formulation can be identical to the first formulation, or can comprises any of the formulations provided herein. i) a cytokine, ii) an antibody, antibody mimetic, or polypeptide that is capable of binding CD3, CD28, OX40, 4-1BB, ICOS, CD9, CD53, CD63, CD81, and/or CD82, and/or iii) a source of the cognate antigen recognized by the CAR, and optionally wherein the cytokine is IL-2, IL-7, IL-15, or IL-21, or a modified version of any of these cytokines that is capable of binding to and activating a native receptor for the cytokine.

In some embodiments of any of the aspects herein that include a cell mixture or cell formulation, any cell in a cell mixture can be enriched. For example, a cell useful in adoptive cell therapy, such as one or more cell populations of T and/or NK cells, can be enriched prior to formulation for delivery. In some embodiments, the one or more cell populations can be enriched after the cell mixture is contacted with a recombinant nucleic acid vector, such as a replication incompetent retroviral particle. In some embodiments, enriching the one or more cell populations can be performed at the same time as any of the methods of genetic modification disclosed herein, and in illustrative embodiments genetic modification with a replication incompetent retroviral particle.

In some embodiments of any of the aspects herein that include mononuclear cells (such as PBMCs) or TNCs, the mononuclear cells or TNCs can be isolated from a more complex cell mixture such as whole blood by density-gradient centrifugation or reverse perfusion of a leukoreduction filter assembly, respectively. In some embodiments, specific cell lineages, such as NK cells, T cells, and/or T cell subsets including naïve, effector, memory, suppressor T-cells, and/or regulatory T cells can be enriched through the selection of cells expressing one or more surface molecules. In illustrative embodiments, the one or more surface molecules can include CD4, CD8, CD16, CD25, CD27, CD28, CD44, CD45RA, CD45RO, CD56, CD62L, CCR7, KIRs, FoxP3, and/or TCR components such as CD3. Methods using beads conjugated to antibodies directed to one or more surface molecules can be used to enrich for the desired cells using magnetic, density, and size-based separation. In the process of such antibody-based positive selection methods, binding of the one or more cell surface molecules can lead to signal transduction and alteration of the biology of the bound cell. For example, selection of T cells using beads with attached antibodies to CD3 may lead to CD3 signal transduction and T cell activation. In other examples, binding and signal transduction may lead to further cell differentiation of cells such as naïve or memory T cells. In some embodiments, positive selection is not used to enrich for desired cells such as when it is preferred that the desired cells are not contacted but rather are left untouched. Any of these methods for positive selection provided in the embodiments in this paragraph can be performed before, during, or after a contacting step.

In some embodiments of any of the aspects herein that include a cell mixture or cell formulation, one or more unwanted cell populations can be depleted, such that the desired cells in the cell mixture or cell formulation are enriched. In some embodiments, the one or more cell populations can be depleted by negative selection prior to being contacted with a recombinant nucleic acid vector, such as a replication incompetent retroviral particle. In some embodiments, the one or more cell populations can be depleted by negative selection after the cell mixture is contacted with a recombinant nucleic acid vector, such as a replication incompetent retroviral particle. In some embodiments, depleting the one or more cell populations can be performed before or at the same time as any of the methods of genetic modification disclosed herein, and in illustrative embodiments genetic modification with a replication incompetent retroviral particle.

In some embodiments, the unwanted cells include cancer cells. Cancer cells from many types of cancer can enter the blood and could be unintentionally genetically modified at a low frequency along with the lymphocytes using the methods provided herein. In some embodiments, the cancer cell can be derived from any cancer, including, but not limited to: renal cell carcinoma, gastric cancer, sarcoma, breast cancer, lymphoma, B cell lymphoma, a B cell lymphoma such as diffuse large B cell lymphoma (DLBCL), Hodgkin's lymphoma, non-Hodgkin's B-cell lymphoma (B-NHL), neuroblastoma, glioma, glioblastoma, medulloblastoma, colorectal cancer, ovarian cancer, prostate cancer, mesothelioma, lung cancer (e g , small cell lung cancer), melanoma, leukemia, chronic lymphocytic leukemia (CLL), acute lymphocytic leukemia (ALL), acute myelogenous leukemia (AML), or chronic myelogenous leukemia (CML), or any of the cancers listed in this disclosure. In illustrative embodiments, the CAR-cancer cell can be derived from a lymphoma, and, in illustrative embodiments, a B-cell lymphoma.

In some embodiments, the unwanted cells can include monocytes. In some embodiments, monocytes can be depleted by incubation of the cell mixture with an immobilized monocyte-binding substrate such as a standard plastic tissue culture plastic, nylon or glass wool or sephadex resin. In some embodiments, the incubations can be performed at 37° C. for at least 1 hour or by passing the cell mixture through a resin. Following incubation, the desired non-adherent cells in suspension are collected for further processing. In illustrative embodiments of rapid ex vivo processing of lymphocytes provided herein, the whole blood, TNCs, or PBMCs are not incubated with an immobilized monocyte-binding substrate.

In some embodiments, the unwanted cells can be depleted by negative selection of cells expressing one or more surface molecules. In illustrative embodiments, the surface molecule is a tumor-associated antigen, a tumor-specific antigen, or is otherwise expressed on cancer cells. In illustrative embodiments, the surface molecules can include Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD19, CD20, CD22, CD23, CD24, CD27, CD30, CD33, CD34, CD37, CD38, CD40, CD44, CD44v6, CD44v7/8, CD45, CD70, CD99, CD117, CD123, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, NYESO1, or PDL-1.

In further illustrative embodiments, the surface molecule is a blood cancer antigen such as CD19, CD20, CD22, CD25, CD32, CD34, CD38, CD123, BCMA, TACI, or TIM3. In some embodiments, the unwanted cells can be depleted from a cell mixture such as whole blood, PBMCs, or TNCs, by bead. In some embodiments, the unwanted cells can be depleted by column-based separation. In these embodiments, ligand or antibody that binds to the cell surface molecule is attached to the beads or column. In some embodiments, the antibodies attached to the beads can bind the same antigen as the CAR. In some embodiments, the antibodies attached to the beads can bind a different epitope of the same antigen as the CAR that will be expressed in the patient. In illustrative embodiments, the antibodies attached to the beads can bind the same epitope of the same antigen as the CAR. In some embodiments, the beads can have more than one attached antibody that binds to antigens on the surface of the unwanted cells. In some embodiments, beads with different antibodies attached to them can be used in combination. In some embodiments, the beads can be magnetic beads. In some embodiments, the unwanted cells can be depleted by magnetic separation after incubation of the cell mixture with the magnetic beads with attached antibodies. In some embodiments, the beads are not magnetic.

In some embodiments, the unwanted cells expressing one or more surface molecules can be depleted from a cell mixture such as whole blood, PBMCs, or TNCs, by antibody coated beads and separated by size. In some embodiments the beads are polystyrene. In illustrative embodiments the beads are at least about 30 μm, about 35 μm, about 40 μm, about 50 μm, about 60 μm, about 70 μm, or about 80 μm in diameter. In some embodiments the antibody coated beads are added to the cell mixture during the time that the recombinant nucleic acid vectors, which in illustrative embodiments are RIPs, are incubated with the cell mixture. In these embodiments, a reaction mixture is formed that includes: (A) a cell mixture, such as from whole blood, enriched TNCs, or isolated PBMCs; (B) recombinant nucleic acid vectors, such as RIPs, encoding a transgene of interest, such as a CAR; and (C) antibody coated beads that bind to one or more surface molecules, or antigens, expressed on the surfaces of the unwanted cells. In some embodiments, the reaction mixture can be incubated for less than 1, 2, 3, 4, 5, 6, 7, 8, 9, 10, 15, 20, 25, 30, or 45 minutes or less than 1, 2, 3, 4, 5, 6, 7, or 8 hours. In some embodiments, after the incubation, a density-gradient centrifugation-based cell enrichment procedure can be performed to enrich total mononuclear cells depleted of the unwanted cells complexed to the antibody coated beads. In other embodiments, the reaction mixture can be passed through a filter to deplete the unwanted cells complexed to the antibody coated beads. In some embodiments, the filter can have a pore diameter that is or is about 5 μm, 10 μm, or 15 μm smaller than the diameter of the beads. Such filters can capture the unwanted cells bound to the beads and allow the desired cells to flow through downstream to the leukoreduction filter assembly which has a smaller pore diameter.

In some embodiments, the unwanted cells can be depleted from a cell mixture that contains lymphocytes and erythrocytes, such as whole blood, by erythrocyte antibody rosetting (EA-rosetting). In some embodiments the antibodies that mediate EA-rosetting are added to the cell mixture during the time that the recombinant nucleic acid vectors, which in illustrative embodiments are RIPs, are incubated with the cell mixture. In illustrative embodiments, a reaction mixture is formed that includes: (A) a cell mixture of lymphocytes and erythrocytes, such as from whole blood; (B) recombinant nucleic acid vectors, such as RIPs, encoding a transgene of interest, and in further illustrative embodiments a CAR; (C) a first antibody to an antigen on the surface of the unwanted cells, for example a tumor antigen such as the blood cancer antigens CD19, CD20, CD22, CD25, CD32, CD34, CD38, CD123, BCMA, TACI, or TIM3; (D) a second antibody to an antigen on the surface of an erythrocyte, such as glycophorin A; and (E) a third antibody that cross links the first and second antibodies. In further illustrative embodiments, the reaction mixture can include antibodies to more than one antigen on the surface of unwanted cells. In some embodiments, the antibodies can bind to the same antigen as does the CAR. In some embodiments, this reaction mixture is incubated for less than 1, 2, 3, 4, 5, 6, 7, 8, 9, 10, 15, 20, 25, 30, or 45 minutes or less than 1, 2, 3, 4, 5, 6, 7, or 8 hours. In illustrative embodiments, after the incubation, a density-gradient centrifugation-based PBMC enrichment procedure is performed to isolate total PBMCs minus the population depleted or removed by EA-rosetting. In illustrative embodiments, after the incubation, a density-gradient centrifugation-based PBMC enrichment procedure is performed to isolate total PBMCs minus the population depleted or removed by EA-rosetting which will pellet with the erythrocytes.

In certain embodiments of any of the aspects herein that include blood cells, the blood cells in the reaction mixture comprise at least 10% neutrophils and at least 0.5% eosinophils, as a percent of the white blood cells in the reaction mixture.

In certain embodiments of any of the aspects herein that include a reaction mixture and/or a cell formulation, the reaction mixture and/or the cell formulation comprises at least 5%, 10%, 20%, 25%, 30%, or 40% neutrophils as a percent of cells in the reaction mixture or cell formulation, or between 20% and 80%, 25% and 75%, or 40% and 60% neutrophils as a percent of white blood cells in the reaction mixture or cell formulation.

In certain embodiments of any of the aspects herein that include a reaction mixture and/or a cell formulation, the reaction mixture and/or the cell formulation comprises at least 0.1% eosinophils, or between 0.25% and 8% eosinophils, or between 0.5% and 4% as a percent of white blood cells in the reaction mixture or cell formulation.

In certain embodiments of any of the aspects herein that include blood cells, the blood cells in the reaction mixture are not subjected to a PBMC enrichment procedure before the contacting.

In certain embodiments of any of the aspects herein that include a reaction mixture, the reaction mixture is formed by adding the recombinant retroviral particles to whole blood.

In certain embodiments of any of the aspects herein that include a reaction mixture, the reaction mixture is formed by adding the recombinant retroviral particles to substantially whole blood comprising an effective amount of an anticoagulant.

In certain embodiments of any of the aspects herein that include a reaction mixture, the reaction mixture is in a closed cell processing system. In certain embodiments of such a reaction mixture, use, modified and in illustrative embodiments genetically modified T cell or NK cell, or method for modifying and/or genetically modifying T cells and/or NK cells, the blood cells in a reaction mixture are whole blood or PBMCs and optionally the reaction mixture is in contact with a leukoreduction filter assembly in the closed cell processing system, and in optional further embodiments the leukoreduction filter assembly comprises a greater than 25 ml volume leukoreduction filter, such as a HemaTrate filter or a 25 ml or less volume leukoreduction filter, such as an Acrodisc filter. In one aspect, provided herein is a composition that includes T cells and/or NK cells, RIPs, and a large volume leukoreduction filter (e.g. Hematrate filter) or a small volume leukoreduction filter (e.g. Acrodisc filter). In another aspect. In some embodiments, the volume of blood sample applied to the large volume leukoreduction filter is 120, 100, 75, 50, 40, 30, 25, 20, 15, 10, or 5 ml or less. In some embodiments, the blood sample is applied to a leukoreduction filter assembly that includes a small volume leukoreduction filter (e.g. Acrodisc filter). In some embodiments, the volume of blood sample applied to the small volume leukoreduction filter is 20, 15, 14, 13, 12, 11, 10, 9, 8, 7, 6, 5, 4, 3, 2, or 1 ml or less, or between 2 ml and 20, 15, 14, 13, 12, 11, 10, 9, 8, 7, 6, 5, 4, and 3 ml.

In certain embodiments of any of the aspects herein that include a reaction mixture, the reaction mixture comprises an anticoagulant. For example, in certain embodiments, the anticoagulant is selected from the group consisting of acid citrate dextrose, EDTA, or heparin. In certain embodiments, the anticoagulant is other than acid citrate dextrose. In certain embodiments, the anticoagulant comprises an effective amount of heparin.

In certain embodiments of any of the aspects herein that include a reaction mixture, the reaction mixture is in a blood bag during the contacting.

In certain embodiments of any of the aspects herein that include a reaction mixture, the reaction mixture is in contact with a T lymphocyte and/or NK cell-enriching filter in the closed cell processing system before the contacting, and wherein the reaction mixture comprises granulocytes, wherein the granulocytes comprise at least 10% of the white blood cells in the reaction mixture, or wherein the reaction mixture comprises at least 10% as many granulocytes as T cells, wherein the modified and in illustrative embodiments genetically modified lymphocytes (e.g. T cells or NK cells) are subject to a PBMC enrichment process after the contacting.

In certain embodiments of any of the aspects herein that include a blood cells in a reaction mixture, blood cells in the reaction mixture are PBMCs and the reaction mixture is in contact with a leukoreduction filter assembly in the closed cell processing system after the contacting comprising an optional incubating in the reaction mixture.

In certain embodiments of any of the aspects herein that include unfractionated whole blood, the unfractionated whole blood is other than cord blood.

In certain embodiments of any of the aspects herein that include a reaction mixture, the reaction mixture is in contact with a leukoreduction filter assembly in a closed cell processing system before the contacting, at the time the recombinant retroviral particles and the blood cells are contacted, during the contacting comprising an optional incubating in the reaction mixture, and/or after the contacting comprising the optional incubating in the reaction mixture, wherein the T cells and/or NK cells, or the modified and in illustrative embodiments genetically modified T cells and/or NK cells are further subjected to a PBMC enrichment procedure.

In certain embodiments of any of the aspects herein that are or include a method, the method further comprises administering the modified T cells and/or NK cells to a subject subcutaneously. Optionally in such certain embodiments, the modified T cells and/or NK cells are delivered in a cell formulation that further comprises neutrophils. Furthermore, optionally in such certain embodiments, the neutrophils are present in the cell formulation at a concentration too high for safe intravenous delivery, and/or the cell formulation comprises 5%, 10%, 15%, 20%, or 25% neutrophils. In some embodiments of any of the methods herein that include a collecting, contacting, and an administrating step, the modified lymphocytes are introduced back into the subject within 14 hours, 12 hours, 8 hours, 6 hours, 4 hours, 2 hours, 1 hour, or 30 minutes from the time the blood comprising the lymphocytes is collected from the subject. In illustrative embodiments, such methods include subcutaneous administration. In illustrative embodiments, such methods include collecting blood cells using apheresis, or filtration of blood cells or modified lymphocytes over a filter, such as a leukoreduction filter.

In some embodiments, the reaction mixture comprises an anticoagulant, wherein the lymphocytes are in unfractionated whole blood from the subject when they are contacted. In some embodiments, the cell formulation comprises between 1×10⁶ and 1×10⁸ modified lymphocytes. In some embodiments, the reaction mixture comprises at least 25% unfractionated whole blood by volume. In some embodiments, the reaction mixture is in a closed cell processing system, wherein the contacting occurs when the reaction mixture is in a leukoreduction filter assembly in the closed cell processing system, and wherein the blood cells in the cell formulation are total nucleated cells (TNCs).

In some embodiments, the T cell and/or NK cell activation element is on the surface of the RIPs, the contacting is performed at between 2° C. and 15° C., and optionally between 2° C. and 6° C., for less than 8 hours, 6 hours, 4 hours, 2 hours, or 1 hour, optionally after which the TNCs are incubated at between 32° C. and 42° C. for between 5 minutes and 4 hours, and optionally after which the modified T cells and/or NK cells are collected on a filter to form the cell formulation. In some embodiments, the reaction mixture comprises at least 25% unfractionated whole blood by volume and an effective amount of an anticoagulant. In some embodiments, the anticoagulant is selected from the group consisting of acid citrate dextrose, EDTA, and heparin. In some embodiments, the anticoagulant is other than acid citrate dextrose. In some embodiments, the anticoagulant comprises an effective amount of heparin.

In certain embodiments of any of the aspects herein that includes a method, the method further comprises administering the modified T cells and/or NK cells to the subject subcutaneously in the presence of a hyaluronidase. In further illustrative subembodiments, the T cells and/or NK cells that were modified, were obtained from the subject.

In further subembodiments of these embodiments including administering the modified and in illustrative embodiments genetically modified T cells and/or NK cells to the subject subcutaneously in the presence of a hyaluronidase, the modified T cells and/or NK cells are delivered subcutaneously to a subject in a volume between 1 ml and 5 ml. In further subembodiments, the T cells and/or NK cells are in blood drawn from a subject, and the modified T cells and/or NK cells are delivered back into the subject, and in further embodiments within 1-14, 1-8 hours, 1-6 hours, 1-4 hours, 1-2 hours, or within 1 hour from the time the blood is drawn from the subject.

In certain embodiments of any of the aspects herein that include a reaction mixture, the reaction mixture is in contact with a leukoreduction filter assembly in a closed cell processing system before the contacting, at the time the recombinant retroviral particles and the blood cells are contacted, during the contacting comprising an optional incubating in the reaction mixture, and/or after the contacting comprising the optional incubating in the reaction mixture.

In some embodiments of any of the aspects herein, at least 10%, 20%, 25%, 30%, 40%, 50%, most, 60%, 70%, 75%, 80%, 90%, 95%, or 99% of the T cells are resting T cells, or of the NK cells are resting NK cells, when they are combined with the replication incompetent retroviral particles to form the reaction mixture.

In any of the aspects herein that include modifying cells, the cell or cells are not subjected to a spinoculation procedure, for example not subjected to a spinoculation of at least 800 g for at least 30 minutes.

In some embodiments of any of the aspects herein that include a method, the method further comprises administering the modified T cells and/or NK cells to a subject, optionally wherein the subject is the source of the blood cells. In some subembodiments of these and embodiments of any of the methods and uses herein, including those in this Exemplary Embodiments section, provided that it is not incompatible with, or already stated, the modified, genetically modified, and/or transduced lymphocyte (e.g. T cell and/or NK cell) or population thereof, undergoes 4 or fewer cell divisions ex vivo prior to being introduced or reintroduced into the subject. In some embodiments, no more than 8 hours, 6 hours, 4 hours, 2 hours, or 1 hour pass(es) between the time blood is collected from the subject and the time the modified lymphocytes are reintroduced into the subject. In some embodiments, all steps after the blood is collected and before the blood is reintroduced, are performed in a closed system, optionally in which a person monitors the closed system throughout the processing. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the solution can include a pseudotyping element or a T cell activating antibody on their surfaces. In some embodiments, the pseudotyping element and/or a T cell activating antibody can be bound to the surface of the modified lymphocytes through, for example, a T cell receptor, and/or the pseudotyping element and/or a T cell activating antibody can be present in the plasma membrane of the modified lymphocytes.

In any of the aspects herein that include genetic modification and/or transduction, an ABC transporter inhibitor and/or substrate, in further subembodiments an exogenous ABC transporter inhibitor and/or substrate, is not present before, during, or both before and during the genetic modification and/or transduction.

In any of the kits provided hereinabove, the first and/or second polynucleotides can comprise any self-driving CAR provided herein. Additional kit aspects and embodiments are provided hereinbelow, and in the Detailed Description herein, outside this Exemplary Embodiments section.

For any of the aspects provided herein that include a syringe, in illustrative embodiments, the syringe is compatible with, effective for, and/or adapted for intramuscular, and in illustrative embodiments subcutaneous delivery, and/or is effective to inject intramuscularly, effective to inject subcutaneously, adapted to inject intramuscularly, and/or adapted to inject subcutaneously. For example, the syringe can have a needle with a gauge between 20 and 22 and a length between 1 inch and 1.5 inches for intramuscular delivery and a needle with a gauge between 26 and 30 and a length between 0.5 inches and 0.625 inches for subcutaneous delivery.

In certain embodiments of any of the aspects and other embodiments herein that comprise a polynucleotide that encodes a CAR and an LE (e.g., polynucleotides, RIPs, cell formulations, populations, genetically modified lymphocytes, reaction mixtures, mammalian packaging cell lines comprising a packageable RNA genome for a replication incompetent retroviral particle, kits, uses of a RIP(s) in the manufacture of a kit for genetically modifying and/or transducing a lymphocyte, methods for genetically modifying and/or transducing a T cell or an NK cell, methods for administering a genetically modified lymphocyte to a subject), the polynucleotide can include or encode any of the self-driving CAR embodiments disclosed in the Self-Driving Car Methods and Compositions section herein.

In some embodiments, the self-driving CAR embodiment can be a polynucleotide comprising a first transcriptional unit operably linked to an inducible promoter inducible in at least one of a T cell or an NK cell, and a second transcriptional unit operably linked to a constitutive T cell or NK cell promoter, wherein the first transcriptional unit and the second transcriptional units are arranged divergently, wherein at the first transcriptional unit encodes an LE, and wherein at the second transcriptional unit encodes a CAR, wherein the CAR comprises an ASTR, a transmembrane domain, and an intracellular activating domain

In some embodiments, the self-driving CAR embodiment can be a polynucleotide comprising a first sequence comprising one or more first transcriptional units operably linked to an inducible promoter inducible in at least one of a T cell or an NK cell, wherein at least one of the one or more first transcriptional units comprises a first polynucleotide sequence encoding a first polypeptide comprising an LE, wherein the lymphoproliferative element is constitutively active in at least one of a T cell or an NK cell, wherein the lymphoproliferative element comprises a transmembrane domain, and wherein the one or more first transcriptional units does not comprise a signal sequence comprising a signal peptidase cleavage site.

In some embodiments, the self-driving CAR embodiment can be a polynucleotide comprising a first sequence in a reverse orientation comprising one or more first transcriptional units operably linked to an inducible promoter inducible in at least one of a T cell or an NK cell, and a second sequence in a forward orientation comprising one or more second transcriptional units operably linked to a constitutive T cell or NK cell promoter, wherein the number of nucleotides between the 5′ end of the one or more first transcriptional units and the 5′ end of the one or more second transcriptional units is less than the number of nucleotides between the 3′ end of the one or more first transcriptional units and the 3′ end of the one or more second transcriptional units, wherein the polynucleotide further comprises a 5′ LTR and a 3′ LTR, and wherein the reverse and forward orientations are relative to the 5′ to 3′ orientation established by the 5′ LTR and the 3′ LTR, wherein at least one of the one or more first transcriptional units encodes an LE, and wherein at least one of the one or more second transcriptional units encodes a CAR, wherein the CAR comprises an ASTR, a transmembrane domain, and an intracellular activating domain

In some embodiments, the self-driving CAR embodiment can be a polynucleotide comprising one or more first transcriptional units operably linked to an inducible promoter inducible in at least one of a T cell or an NK cell, and one or more second transcriptional units operably linked to a constitutive T cell or NK cell promoter, wherein the number of nucleotides between the 5′ end of the one or more first transcriptional units and the 5′ end of the one or more second transcriptional units is less than the number of nucleotides between the 3′ end of the one or more first transcriptional units and the 3′ end of the one or more second transcriptional units, wherein at least one of the one or more first transcriptional units encodes an LE, and wherein at least one of the one or more second transcriptional units encodes a CAR, wherein the CAR comprises an ASTR, a transmembrane domain, and an intracellular activating domain.

In some embodiments, for any aspects that include a polynucleotide including one or more first transcriptional units operably linked to an inducible promoter where at least one of the one or more first transcriptional units encodes an LE, the polynucleotide can further comprise a second sequence comprising one or more second transcriptional units operably linked to a constitutive T cell or NK cell promoter, wherein at least one of the one or more second transcriptional units comprises a second polynucleotide sequence encoding a second polypeptide comprising a CAR, wherein the CAR comprises an ASTR, a transmembrane domain, and an intracellular activating domain In some embodiments, the inducible promoter is an NFAT-responsive promoter. In some embodiments, the first and second transcriptional units are separated by the 250 cHS4 insulator (SEQ ID NO:358) in the forward orientation. In some embodiments, the RIPs are lentiviral particles.

Further details and embodiments, to be used in any combination with any of the self-driving CAR embodiments in the preceding paragraphs, are disclosed in the SELF-DRIVING CAR METHODS AND COMPOSITIONS section herein.

In any of the aspects herein that include recombinant retroviral particles in a container and/or reaction mixture, the recombinant retroviral particles are present in the container and/or reaction mixture at an MOI of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15 or are present in the reaction mixture at an MOI of at least 0.1, 0.5, 1, 2, 2.5, 3, 5, 10 or 15. For kit and isolated retroviral particle embodiments, such MOI can based on 1, 2.5, 5, 10, 20, 25, 50, 100, 250, 500, or 1,000 ml assuming 1×10⁶ target cells/ml, for example in the case of whole blood, assuming 1×10⁶ PBMCs/ml of blood.

In any of the aspects herein that include a contacting cells with retroviral particles, sufficient retroviral particles are present in a reaction to achieve an MOI of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15, or to achieve an MOI of at least 0.1, 0.5, 1, 2, 2.5, 3, 5, 10 or 15.

In any of the aspects herein that include a genetically modified T cell and/or NK cell, at least 5%, at least 10%, at least 15%, or at least 20% of the T cells and/or NK cells are genetically modified, or between 5% and 85%, or between 5% and 20%, 25%, 50%, 60%, 70%, 80%, or 85%, or between 5%, 10%, 15%, 20%, or 25% on the low end of the range, and 20%, 25%, 50%, 60%, 70%, 80%, or 85% on the high end of the range.

In any of the aspects herein that include, RIPs, the RIPs are lentiviral particles. In further illustrative embodiments, the modified cell is a modified T cell or a modified NKT cell.

In any of the aspects herein that include a polynucleotide including one or more transcriptional units, the one or more transcriptional units can encode a polypeptide comprising a CAR. In some embodiments, the CAR is a microenvironment restricted biologic (MRB)-CAR. In other embodiments, the ASTR of the CAR binds to a tumor associated antigen. In other embodiments, the ASTR of the CAR is a microenvironment-restricted biologic (MRB)-ASTR.

In certain embodiments, any of the aspects and embodiments provided herein that include a polynucleotide that comprises a nucleic acid sequences operatively linked to a promoter active in T cells and/or NK cells, the polynucleotide encodes at least one polypeptide lymphoproliferative element. In illustrative embodiments, the polypeptide lymphoproliferative element is any of the polypeptide lymphoproliferative elements disclosed herein. In some embodiments, any or all of the nucleic acid sequences provided herein can be operably linked to a riboswitch. In some embodiments, the riboswitch is capable of binding a nucleoside analog. In some embodiments, the nucleoside analog is an antiviral drug.

In any of the aspects provided herein that include a RIP, in some embodiments, the RIP comprises on its surface a nucleic acid encoding a domain recognized by a monoclonal antibody approved biologic.

In certain illustrative embodiments of any of the aspects herein that include blood cells in a reaction mixture, the blood cells in the reaction mixture are blood cells that were produced by a PBMC enrichment procedure and comprise PBMCs, or the blood cells in illustrative embodiments are PBMCs. In illustrative embodiments, such embodiments including PMBC enrichment are not combined with an embodiment where the reaction mixture includes at least 10% whole blood. Thus, in certain illustrative embodiments herein, the blood cells in a reaction mixture are the PBMC cell fraction from a PBMC enrichment procedure to which retroviral particles are added to form the reaction mixture, and in other illustrative embodiments, the blood cells in a reaction mixture are from whole blood to which retroviral particles are added to form the reaction mixture.

In any of the aspects and embodiments provided herein that include, or optionally include, a nucleic acid sequence encoding an inhibitory RNA molecule, the inhibitory RNA molecule targets any of the gene (e.g. mRNAs encoding) targets identified for example in the Inhibitory RNA Molecules section herein.

In illustrative embodiments of any of the kits, delivery solutions and/or cell formulations provided herein, especially those that effective for, or adapted for intramuscular and in illustrative embodiments subcutaneous delivery, the delivery solution and/or cell formulation is a depot formulation, or the cell formulation is an emulsion of cells that promotes cell aggregation. In some embodiments, a depot delivery solution comprises an effective amount of alginate, hydrogel, PLGA, a cross-linked and/or polymer hyaluronan, PEG, collagen, and/or dextran to form a depot formulation. In some embodiments the delivery solution and/or cell formulation is designed for controlled or delayed release. In some embodiments, the delivery solution and/or cell formulation includes components that form an artificial extracellular matrix such as a hydrogel.

In any of the aspects and embodiments provided herein that include, or optionally include, a cell mixture, delivery solution, or cell formulation, the cell mixture, delivery solution, or cell formulation can have a pH and ionic composition that provides an environment in which cells can survive, for example for at least 1 hour, and typically can survive for at least 4 hours. In some embodiments, the pH can be between pH 6.5 to pH 8.0, pH 7.0 to pH 8.0, or pH 7.2 to pH 7.6. In some embodiments, for example, when the RIP has a polynucleotide that encodes an MRB-CAR, the pH can be between pH 6.0 to pH 7.0, for example, pH 6.2 to pH 7.0, or pH 6.4 to pH 7.0, or pH 6.4 to pH 6.8. In some embodiments, the cell mixture, delivery solution, or cell formulation can be maintained by a buffer such as a phosphate buffer or bicarbonate present at a concentration effective for maintaining pH in a target range. In some embodiments, a cell mixture, delivery solution, or cell formulation can include a saline composition with salts, for example 0.8 to 1.0 or about 0.9 or 0.9 percent salts such as sodium chloride. In some embodiments, the delivery solution is or includes PBS. In some embodiments of a delivery solution and resulting cell formulation herein, the concentration of Na⁺ is between 110 mM and 204 mM, the concentration of Cl⁻ is between 98 mM and 122 mM, and/or the concentration of K⁺ is between 3 mM and 6 mM.

In illustrative embodiments of any of the kits, delivery solutions and/or cell formulations provided herein, especially those that effective for, or adapted for intramuscular and in illustrative embodiments subcutaneous delivery, the delivery solution comprises one or more components disclosed herein, such as a molecule(s) (ion(s)), macromolecule(s) (e.g. DNA, RNA, peptides, and polypeptides) and/or other cell(s), that can affect genetically modified T cells and/or NK cells such as the genetically modified T cells and/or NK cells provided herein. Accordingly, in some embodiments a delivery solution and/or cell formulation provided herein, comprises an effective amount of an antigen as discussed in further detail herein. In some embodiments, such formulations do not include genetically modified T cell and/or NK cells. In illustrative embodiments, such formulations include or are co-administered with formulations that include, genetically modified T cells and/or NK cells, especially those provided in aspects and other embodiments herein. In some embodiments a delivery solution and/or cell formulation provided herein includes an effective amount of one or more cytokines such as IL-2, IL-7, IL-15, IL-21, or a modified version thereof that is adapted for subcutaneous delivery and/or retains cytokine activity. In illustrative embodiments such modified cytokine is capable of binding to and activating a native receptor (e.g., wild-type receptor) for the cytokine. In illustrative embodiments, a modified cytokine has preferentially biased cytokine activity. In some embodiments the cell formulation and/or delivery solution includes an effective amount of antibodies or polypeptides that are capable of binding CD2, CD3, CD28, OX40, 4-1BB, ICOS, CD9, CD53, CD63, CD81, and/or CD82. In some embodiments, these cytokines, antibodies, or polypeptides are crosslinked to components of a hydrogel. In some embodiments, modified T cells delivered along with such other components comprise a polynucleotide encoding CAR and an LE, and in illustrative embodiments the polynucleotides encodes a CAR but not a lymphoproliferative element. In illustrative embodiments, the delivery solution and/or cell formulation lacks DMSO and was never frozen. In some embodiments, the cell formulation is within a delivery device compatible with, adapted for, or operative for instramuscular or subcutaneous delivery to a human subject. In some embodiments, such a device has a needle with sizes effective for delivery of cells intramuscularly or subcutaneously as provided herein. Once delivered subcutaneously, the subcutaneous formulations in any of the aspects and embodiments provided herein form a subcutaneous reaction mixture comprising modified lymphocytes and/or TILs. In one aspect, provided herein is a subcutaneous reaction mixture comprising any of the modified lymphocytes provided herein and one or more of the other cell formulation components provided herein. Thus, in some aspects, provided herein is a subcutaneous reaction mixture comprising a modified T cell and/or NK cell, or genetically modified T cell and/or NK cell provided herein, and/or a TIL and i) a cytokine, ii) an antibody or polypeptide that is capable of binding CD2, CD3, CD28, OX40, 4-1BB, ICOS, CD9, CD53, CD63, CD81, and/or CD82, and/or iii) a source of the cognate antigen recognized by the CAR. Such compositions can comprise any of the other or specific subcutaneous formulation components provided herein. In some embodiments, the subcutaneous reaction mixture comprises neutrophils. In some embodiments, the subcutaneous reaction mixture comprises an artificial matrix. In some embodiments, the artificial matrix comprises hyaluronic acid and/or collagen. In some embodiments, at least 25%, 50%, 75%, or 90% of the CD4+ cells and/or CD8+ cells in the subcutaneous reaction mixture are surface CD3−

In illustrative embodiments, a delivery solution and cell formulation comprising the same, contains calcium and/or magnesium. The concentration of calcium can, for example, be between 0.5 mM and 2 mM. The concentration of magnesium can, for example, be between 0.5 mM and 2 mM. In some embodiments, the delivery solution is calcium and magnesium free.

In some embodiments, the delivery solution and cell formulations contain human serum albumin and/or heparin. In some embodiments the delivery solution and cell formulation contains up to 5% HSA. In some embodiments the delivery solution is PBS comprising 2% HSA. In some embodiments, the delivery solution is DPBS comprising 2% HSA. In some embodiments, the delivery solution is a saline solution comprising 30-100 U/ml, 40-100 U/ml, 30-60 U/ml, or 60-80 U/ml heparin, with or without 0.5-5%, 1-5%, or 1-2.5% HSA.

In some embodiments, the delivery solution is or includes a multiple electrolyte solution suitable for injection into a subject. In some embodiments, a delivery solution can be or include a sterile, nonpyrogenic isotonic solution in a container, such as a single dose container. Such solution in certain embodiments is suitable or adapted for intravenous administration or intraperitoneal administration as well as subcutaneous and/or intramuscular administration. In some embodiments, a delivery solution can include a multiple analyte solution for injection into a subject where ach 100 mL contains 526 mg of Sodium Chloride, USP (NaCl); 502 mg of Sodium Gluconate (C₆H₁₁NaO₇); 368 mg of Sodium Acetate Trihydrate, USP (C₂H₃NaO₂.3H₂O); 37 mg of Potassium Chloride, USP (KCl); and 30 mg of Magnesium Chloride, USP (MgCl₂.6H₂O) with a pH adjusted to 7.4 (6.5 to 8.0). In illustrative embodiments, the delivery solution contains no antimicrobial agents. In some embodiments, the pH is adjusted with sodium hydroxide. In some embodiments, a multiple electrolyte injection solution can be PLASMA-LYTE A Injection pH 7.4 available from various commercial suppliers.

In some embodiments, the delivery solution or cell formulation includes components that form an artificial extracellular matrix such as a hydrogel. In some embodiments, a depot delivery solution comprises an effective amount of alginate, collagen, and/or dextran to form a depot formulation. In some embodiments, the polymers used to make gel-forming biomaterials, and can be included in delivery solutions and cell formulations provided herein, is composed of poly(ethylene glycol) (PEG) and its copolymers with aliphatic polyesters, such as poly(lactic acid) (PLA), poly(D,L-lactic-co-glycolic acid) (PLGA), poly(ε-caprolactone) (PCL) and polyphosphazenes. In some embodiments, the polymers used to make gel-forming biomaterials, and can be included in delivery solutions and cell formulations provided herein, include thermosensitive triblock copolymers based on poly(N-(2-hydroxypropyl methacrylamide lactate) and poly(ethylenglycol) (p(HPMAm-lac)-PEG), capable of spontaneous self-assembling in physiological environments (Vermonden et. al 2006, Langmuir 22: 10180-10184).

In some embodiments, the hydrogel used in a delivery solution or cell formulation herein, contains hyaluronic acid (HA). Such HA can have carboxylic acid groups that can be modified with 1-ethyl-3-(3-dimethyl aminopropyl)-1-carbodiimide hydrochloride to react with amine groups on proteins, peptides, polymers, and linkers, such as those found on modified lymphocytes provided herein, preferentially in the presence of N-hydroxysuccinimide In some embodiments, antibodies, cytokines and peptides are chemically conjugated to HA using such methods to produce a hydrogel for co-injection as a cell emulsion in some cell formulation embodiments provided herein. Additionally, in some embodiments, HA in delivery solutions and cell formulations is a polymer (e.g. Healon) and/or are crosslinked (e.g. restylane (Abbive/Allergan)), for example lightly crosslinked, through its —OH groups with agents such as glutaraldehyde to reduce the local catabolism of the material following subcutaneous injection. In some embodiments, the HA used in delivery solutions and cell formulations herein, can be of variable length and viscosity. In some embodiments, the HA used in delivery solutions and cell formulations herein, can further be crosslinked with other glycosaminoglycans such as chondroitin sulfate (e.g. Viscoat) or polymers or surfactants. A skilled artisan will recognize that the porosity of the matrix and degree of crosslinking can be regulated to ensure cells, such as modified lymphocytes herein, are capable of migration through the hydrogels. Accordingly, a matrix, such as a hydrogel matrix, when used in a cell formulation herein, can be configured for, or adapted to permit migration of cells through the matrix. In some embodiments, the shear modulus is or is about 2.5 kPa, about 3 kPa, about 3.5 kPa, or about 4 kPa.

In some embodiments, the cell formulation comprises blood cells that have been depleted, or substantially depleted, or wherein at least 50, 60, 75, 80, 90, 95, or 99% of cells have been depleted, that express a target antigen. In some embodiments, the target antigen is the antigen recognized by the CAR. In some embodiments, the cells are depleted using any of the depletion methods provided herein.

In some embodiments, the cell formulation is formulated with a second modified lymphocyte, or population thereof, associated with a recombinant nucleic acid vector, in illustrative embodiments a recombinant retroviral particle, comprising a polynucleotide comprising one or more transcriptional units operatively linked to a promoter active in T cells and/or NK cells, or genetically modified with the polynucleotide, wherein the one or more transcriptional units encode a second polypeptide comprising a second CAR that recognizes a different epitope of the tumor antigen recognized by the first CAR or recognizes a different tumor antigen than the first CAR. In illustrative embodiments, the modified lymphocytes comprise modified T cells and/or NK cells,

In some embodiments, provided herein is a pair of cell formulations, or a use of a pair of recombinant nucleic acid vectors, in illustrative embodiments, replication incompetent retroviral particles to make such a pair of cell formulations, wherein each cell formulation of the pair of cell formulations is formulated with a population of modified lymphocytes, each population associated with a different recombinant nucleic acid vector, in illustrative embodiments a different recombinant retroviral particle, each population comprising a different polynucleotide comprising one or more transcriptional units operatively linked to a promoter active in T cells and/or NK cells, or genetically modified with the polynucleotide, wherein the one or more transcriptional units for each population encodes a different polypeptide comprising a different CAR that recognizes a different epitope of the same tumor antigen or each recognizes a different tumor antigen

In some embodiments, a delivery solution and/or cell formulation provided herein comprises an aggregating agent as provided herein. In some embodiment, a delivery solution and/or cell formulation comprises a cellular matrix, such as a hyaluronic acid matrix and/or a collagen matrix. Such cell formulation can be an ex vivo cell formulation or an in vivo cell formulation localized within a muscle or subcutaneously within a subject. In illustrative embodiments, the hyaluronic acid and/or collagen matrix are localized subcutaneously and in some embodiments, such matrix is the natural subcutaneous matrix found in the subject. Such a matrix found or localized subcutaneously in a subject when including exogenous lymphocytes such as tumor infiltrating lymphocytes and/or modified lymphocytes as provided herein, optionally including other cell formulation components provided herein, can be considered an artificial lymph node. As such, methods provided herein for administering cell formulations to a subject subcutaneously, where the cell formulations comprise an aggregating agent and/or a cellular matrix and/or where a matrix comprising only a subject's natural matrix components is formed around modified lymphocytes delivered subcutaneously, can be referred to as methods for forming an artificial lymph node, and such resulting structures can be considered artificial lymph nodes. In some embodiments the composition comprises modified T cells and/or NK cells, and/or TILs in an artificial matrix, such as a hyaluronic acid and/or collagen matrix, that is located subcutaneous.

In some embodiments, the unwanted cells can be epitope-masking target cells that express both a CAR and the antigen the CAR binds to. In some embodiments, the epitope-masking target cells can be depleted, removed, or killed by contacting them with CAR-T cells expressing a CAR to a different epitope or antigen that the target cells do not mask in a method provided herein, after genetically modifying the cells using methods provided herein. Such first CAR and second CAR in these embodiments, can be referred to as a CAR-pair. In some embodiments, cells expressing two or more separate CARs, and in illustrative embodiments two CARs expressed in two populations of cells, can be used to kill the epitope-masking target cells that are masking only one of the epitopes. In some embodiments, the two populations of cells are transduced or transfected separately so each population expresses either a first CAR or a second CAR. In illustrative embodiments, the epitope-masking target cell expressing the first or second CAR does not mask the epitope that the second and first CAR, respectively, bind to. In some embodiments, the first and second CARs can bind to different epitopes of the same antigen expressed on the epitope-masking target cell. In other embodiments, the first and second CARs can bind to different antigens expressed on the same epitope-masking target cell, including any of the antigens disclosed elsewhere herein. In some embodiments, the first and second CARs can bind to different epitopes of, or different antigens selected from CD19, CD20, CD22, CD25, CD32, CD34, CD38, CD123, BCMA, TACI, or TIM3. In some embodiments, two containers containing separate polynucleotides, each of which encodes one of the CARs of a CAR pair directed to two different epitopes or antigens expressed on the same target cell, are provided in kits herein. In other embodiments, one CAR can be an extracellular ligand or receptor binding to a cancer antigen and the other can be a CAR derived from an antibody fragment. In other embodiments both CARs can be an extracellular ligand or receptor against a different cancer antigen. In one example the CAR is BCMA and April is the ligand binding protein to TACI and BCMA receptors. In further illustrative embodiments, the first CAR can bind to CD19 and the second CAR can bind to CD22, both of which are expressed on B cells and lymphomas. In illustrative embodiments, the modified cell population expressing the first CAR and the the modified cell population expressing the second CAR are formulated separately. In some embodiments, the separate cell formulations are introduced or reintroduced back into the subject at different sites. In some embodiments, separate cell formulations are separately introduced or reintroduced back into the subject at the same site. In other embodiments, the modified cell populations are combined into one formulation that is optionally introduced or reintroduced back into the subject. In illustrative embodiments wherein the cell populations are combined, the cell populations are not combined until after a washing step in which the cells are washed away from the recombinant nucleic acid vectors.

In some embodiments of any of the aspects herein that include a modified or genetically modified T cell or NK cell, or a kit or composition for producing the same, the proliferation and survival of genetically modified T cells and/or NK cells expressing a CAR can be promoted by adding an antigen to which an ASTR of a CAR binds, to a composition, such as a cell formulation, or environment, such as a subcutaneous environment or an intramuscular environment, comprising the genetically modified T cells and/or NK cells. In certain illustrative embodiments, the genetically modified T cell and/or NK cells are genetically modified with a nucleic acid encoding a CAR, but not with a nucleic acid encoding an LE. In some embodiments, the antigen can be added to a cell formulation comprising, or co-administered with, modified and/or genetically modified T cells and/or NK cells in cell formulations and methods provided herein. In some embodiments, the antigen is a protein antigen. In some embodiments, the antigen is mRNA encoding the protein antigen. In some embodiments, the antigen can be soluble. In some embodiments, the antigen can be immobilized on a surface of an artificial matrix, such as a hydrogel. In illustrative embodiments, the antigen can be expressed on the surface of a target cell. In some embodiments, such target cells are present in large numbers in whole blood and are naturally present in the cell formulation without having to be added. In some embodiments, B cells present in whole blood, isolated TNCs, and isolated PBMCs naturally present in the cell formulation can be target cells for T cells and/or NK cells expressing a CAR directed to CD19 or CD22, which are both expressed on B cells. In other embodiments, such target cells are not present in whole blood or are not present in large numbers in whole blood and need to be added exogenously to a cell formulation provided herein. In some embodiments, target cells can be isolated or enriched from a subject, such as from a tumor sample, using methods known in the art. In other embodiments, cells from the subject are modified to express a target antigen. In illustrative embodiments, the antigen expressed on the target cell can include all or a portion of the protein that contains the antigen. In further illustrative embodiments, the antigen expressed on the target cell can include all or a portion of the extracellular domain of the protein that includes the antigen. In some embodiments, the antigen expressed on the target cell can be a fusion with a transmembrane domain that anchors it to the cell surface. In some embodiments, any of the transmembrane domains disclosed elsewhere herein can be used. In some embodiments, the antigen expressed on the target cell can be a fusion with a stalk domain In some embodiments, any of the stalk domains disclosed elsewhere herein can be used. In illustrative embodiments, the antigen can be a fusion with a CD8 stalk and transmembrane domain (SEQ ID NO:24).

In some embodiments, cells in a first cell mixture, and in illustrative embodiments cells in a first cell mixture from the subject, are modified with a recombinant nucleic acid vector encoding an antigen, and cells in a separate second cell mixture from a subject, and in illustrative embodiments cells in a second mixture from the same subject, are modified to express a CAR that binds the antigen. In further illustrative embodiments, either or both of the cell mixtures is whole blood, isolated TNCs, or isolated PBMCs. In illustrative embodiments, the first cell mixture can be modified with a recombinant nucleic acid vector encoding a fusion protein of the extracellular domain of Her2 and the transmembrane domain of PDGF and the second cell mixture can be modified with a recombinant nucleic acid vector encoding a CAR directed to HER2. The cells can then be formulated into a delivery solution to form a cell formulation. Thus, in one aspect, provided herein is a pair of such cell mixtures, or a pair of cell formulations, each comprising one of the cell mixtures or cell formulations, typically physically separated in any of the vessels such as cell bags, provided herein for holding cell formulations. Optionally, the cell formulations are administered to the subject at varying CAR effector cell-to-target-cell ratios. In some embodiments, the effector-to-target ratio at the time of formulation or administration is or is about 10:1, about 9:1, about 8:1, about 7:1, about 6:1, about 5:1, about 4:1, about 3:1, about 2;1, about 1:1, about 1:2, about 1:3, about 1:5, about 1:6, about 1:7, about 1:8, about 1:9, or about 1:10. In illustrative embodiments, antigen is co-administered with the modified T and/or NK cells subcutaneously or intramuscularly.

In some embodiments of any of the aspects herein that include a modified or genetically modified T cell or NK cell, or methods, compositions, and kits for genetically modifying T cells and/or NK cells, the proliferation and survival of genetically modified T cells and/or NK cells expressing a CAR can be promoted by cross-linking CAR molecule within a genetically modified T cell or NK cell, in the absence of the CAR molecules binding to their cognate antigens. Thus, in some embodiments, a T cell or NK cell can comprise an epitope tag bound by an antibody and cross-linked to an epitope tag of a second CAR on the same T cell or NK cell. In some embodiments, the extracellular domain of the CAR can include the epitope tag. In illustrative embodiments, the epitope tag can be in the stalk domain In some embodiments, the epitope tag can be HisS (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), HA Tag (YPYDVPDYA; SEQ ID NO:73), RYIRS (SEQ ID NO:79), Phe-His-His-Thr (SEQ ID NO:80), or WEAAAREACCRECCARA (SEQ ID NO:81). In illustrative embodiments, the epitope tag can be the HisX6 tag (SEQ ID NO:77). In some embodiments, the CARs can be cross-linked and activated by adding soluble antibodies or antibody mimetics that bind the epitope tag, or in illustrative embodiments by adding cells, also referred to herein as universal feeder cells, expressing antibodies or antibody mimetics on their surfaces that bind the epitope tag,. In some embodiments, the same universal feeder cells, for example universal feeder cells expressing an anti-HisX6 antibody, can be used with cells that express CARs that bind to different antigens but that include the same epitope tag, for example HisX6. In some embodiments, the CARs can be cross-linked and activated by adding mRNA that encodes for one or more antibodies or antibody mimetics that bind the epitope tag. The mRNA may encode for antibodies or antibody mimetics that are soluble, membrane-bound, or both soluble and membrane-bound in some embodiments.

Provided herein in one aspect is a cell formulation (i.e., delivery composition), comprising a delivery solution formulated with tumor infiltrating lymphocytes (TILs) and/or modified or unmodified lymphocytes, in illustrative embodiments T cells and/or NK cells, wherein the cell formulation is compatible with, effective for, and/or adapted for subcutaneous or intramuscular delivery. In some embodiments for any of the cell formulations provided herein, the cell formulation is localized subcutaneously, or most of the cell formulation is localized subcutaneously, in a subject. In some embodiments, the cell formulation is localized subcutaneously or intramuscularly, or most of the cell formulation is localized subcutaneously or intramuscularly, in a subject. In some embodiments, wherein the cell formulation comprises TILs, the cell formulation can further comprise modified lymphocytes modified by either or both, being associated with a recombinant nucleic acid vectors, in illustrative embodiments a RIP, comprising a polynucleotide comprising one or more transcriptional units operatively linked to a promoter active in T cells and/or NK cells, or by being genetically modified with the polynucleotide, wherein the one or more transcriptional units encode a first polypeptide comprising a first CAR. In some embodiments, wherein the cell formulation comprises TILs, the cell formulation further comprises a source of a tumor antigen recognized by the TILs. In some embodiments, the TILs are contacted with a nucleic acid vector.

In addition to any of the method aspects and embodiments provided herein, further provided herein are use aspects and embodiments, comprising use of a kit for performing the method, or use of nucleic acid vectors, in illustrative embodiments RIPs, in the manufacture of a kit for performing the method, wherein the use of the kit is to perform the steps of the method aspects or embodiments. For example, in one aspect, provided herein is a method for preparing a cell formulation comprising the C/F steps that comprise nucleic acid vectors, and in illustrative embodiments RIPs, in the contacting step. Such methods can optionally include the administering step above or any administering step herein. Accordingly, further provided herein is use of nucleic acid vectors, and in illustrative embodiments replication incompetent recombinant retroviral particles, in the manufacture of a kit for preparing a cell formulation, wherein use of the kit comprises performing the C/F and optional “A” steps. Similarly, for any use aspects and embodiments provided herein, further provided herein are method aspects and embodiments, comprising the method as recited in the use aspects or embodiments.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1. Materials and Methods for Transduction Experiments

This Example provides materials and methods used in experiments disclosed in subsequent Examples herein.

### Recombinant Lentiviral Particle Production by Transient Transfection.

293T cells (Lenti-X™ 293T, Clontech) were adapted to chemically defined suspension culture by serial expansion in Freestyle™ 293 Expression Medium (animal origin-free, chemically defined, and protein-free), (ThermoFisher Scientific) followed by repeated single cell by serial dilution in 96 well plates to generate a master and working cell bank of cells named F1XT cells, and were used as the packaging cells for experiments herein unless noted otherwise.

Where noted, a typical 4 vector packaging system included 3 packaging plasmids that encoded (i) gag/pol, (ii) rev, and (iii) a pseudotyping element such as VSV-G. The 4^{th} vector of this packaging system is the genomic plasmid, a third generation lentiviral expression vector (containing a deletion in the 3′ LTR leading to self-inactivation) that encoded 1 or more genes of interest. For transfections using 4 plasmids, the total DNA used (1 μg/mL of culture volume) was a mixture of the 4 plasmids at the following molar ratios: 1× gag/pol-containing plasmid, 1× Rev-containing plasmid, 1× viral envelope containing plasmid (VSV-G unless noted otherwise), and 2x genomic plasmid unless noted otherwise. Where noted, a typical 5 vector packaging system was used in which a 5^{th} vector encoding, for example, a T cell activation element such as antiCD3-scFvFc-GPI, was added to the otherwise 4 vector packaging system. For transfections using 5 plasmids, the total DNA used (1 μg/mL of culture volume) was a mixture of the 5 plasmids at the following molar ratios: 1× gag/pol-containing plasmid, 1× Rev-containing plasmid, 1× VSV-G containing plasmid, 2× genomic plasmid, and 1× of the 5^{th} vector unless noted otherwise.

For small-scale (3 ml) lentivirus production, plasmid DNA was dissolved in either 1.5 ml Gibco™ Opti-MEM™ growth media for every 30 mL of culture containing packaging cells in Freestyle™ 293 Expression Medium. Polyethylenimine (PEI) (Polysciences) (dissolved in weak acid) was diluted in 1.5 ml Gibco™ Opti-MEM™ to 2 μg/ml. A 3 ml mixture of PEI and DNA was made by combining the two prepared reagents at a ratio of 2 ug of PEI to 1 ug of DNA. After a 5-minute room temperature incubation, the two solutions were mixed together thoroughly, and incubated at room temperature for 20 more minutes. The final volume (3 ml) was added to 30 ml of packaging cells in suspension at 1×10⁶ cells/ml in a 125 ml Erlenmeyer flask. The cells were then incubated at 37° C. for 72 hours with rotation at 125 rpm and with 8% CO₂ for transfection. For larger-scale lentivirus production (6.6 to 10 L), volumes and ratios of reagents were increased proportionally to support transfection and fermentation in larger reactors of F1XT cells that had been expanded through Erlenmeyer flasks of increasing size until final reactor inoculation and addition of transfection material when cells had reached 1×10⁶ cells/ml. Retroviral particles made by all of these methods are free of non-human derived animal proteins.

After 72 hours, for small scale lentivirus production, the supernatants were harvested and clarified by centrifugation at 1,200 g for 10 minutes. The clarified supernatants were sterile-filtered into a new container. Substantially purified virus was obtained from these clarified supernatants by addition of polyethylene glycol (PEG) followed by centrifugation. For PEG precipitation, ¼ volume PEG (Takara Lenti-X™ Concentrator) was added to the clarified supernatant and incubated overnight at 4° C. The mixture was then centrifuged at 1600g for 1 hour (for 50 ml conical tubes) or 1800 g for 1.5 hours (for 500 ml conical tubes). The supernatant was discarded, and the lentiviral particle pellets were resuspended in 1:100 of the initial volume of packaging cell culture.

For larger scale purification by depth filtration, culture media was harvested 72 hours after addition of transfection solution and clarified by depth filtration using Sartorius (#5445306G9 or #5445306G8) or Millipore (#MCE50027H1) depth filter cartridges using a peristaltic pump. Clarified media was then concentrated using a 500 Kd mPES Hollow Fiber TFF Module (Spectrum) on a KrossFlow TFF System (Spectrum) with a TMP of 2.0+/−0.5 PSI. Following addition of MgCl₂ to 2 mM final volume, Benzonase (EMD Millipore) was added to 50 U/ml to fragment residual DNA. The concentrate was then recirculated followed by diafiltration using 10 volumes of PBS 4% Lactose. The substantially purified concentrated and formulated virus was then sterile-filtered and frozen for use. In other cases, the benzonase was added first to the culture media 24 hours post transfection and the post depth-filtered material was diluted with concentrated Tris NaCl to 50 mM Tris 300 mM NaCl pH 8.0 final. Following loading on a Mustang-Q resin (Pall) and elution with 2 M NaCl, the virus was diluted with PBS Lactose and processed by TFF per above.

Lentiviral particles were titered by serial dilution and analysis of transgene expression, by transduction into 293T and/or Jurkat cells and analysis of transgene expression by FACS or qPCR for lentiviral genome using Lenti-X™ qRT-PCR Titration Kit (#631235) or p24 assay ELISA kit from Takara (Lenti-X™ p24 Rapid Titer Kit #632200). Copy number was calibrated against a plasmid standard containing target sequences for lentivirus and human RNAseP.

### Genomic Plasmids Used in Examples.

The following lentiviral genomic vectors encode genes and features of interest as indicated:

F1-3-23 encodes a CD19 CAR comprised of an anti-CD19scFv, a CD8 stalk and transmembrane region, and an intracellular domain from CD3z followed by T2A and an eTag (aCD19:CD8:CD3z-T2A-eTag).

F1-3-247 encodes a CD19 CAR and a polypeptide lymphoproliferative element comprised from amino to carboxy terminus of the Kozak-type sequence GCCGCCACCAT/UG(G) (SEQ ID NO:331), having the T at the “T/U” residue and having the optional last G, the CD8 signal peptide MALPVTALLLPLALLLHAARP (SEQ ID NO:72) (in which the sequence ATGG from the Kozak-type sequence also encodes the first four nucleotides of the CD8 signal peptide), a FLAG-TAG (DYKDDDDK; SEQ ID NO:74), a linker (GSTSGS; SEQ ID NO:349), an anti-CD19scFv, a CD8 stalk and transmembrane region, and an intracellular domain from CD3z followed by T2A and the lymphoproliferative element comprising the parts E006-T016-S186-S050 which encode an extracellular domain containing a variant of c-Jun including a leucine zipper motif and an eTAG, the transmembrane domain of CSF2RA, the intracellular domain of MPL, and the intracellular domain of CD40 with each part of the lymphoproliferative element connected by a GGS linker.

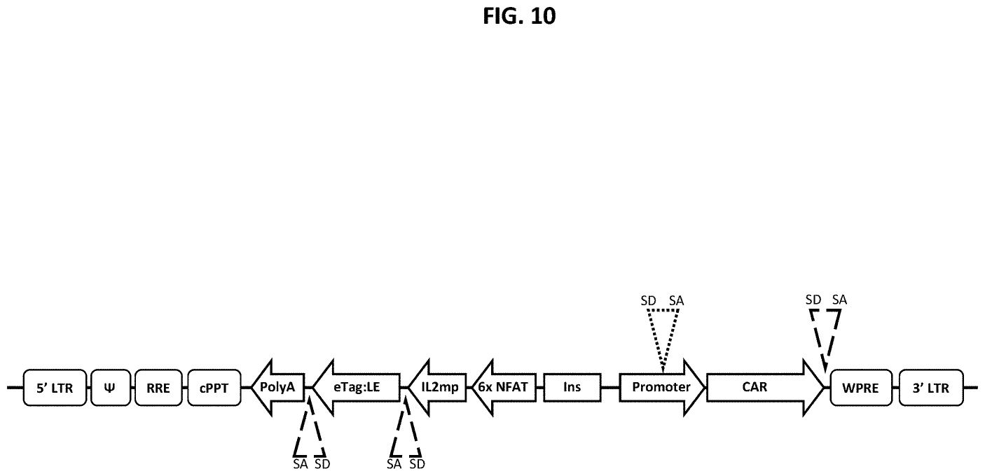

F1-3-635 encodes a bicistronic lentiviral genomic vector with divergent transcriptional units with the general structure shown in the schematic in FIG. 10. The first transcriptional unit encodes the lymphoproliferative element E006-T016-S186-S050 under the control of an NFAT-responsive minimal IL-2 promoter all encoded in the reverse orientation. The second transcriptional unit encodes CD19 CAR comprised of an anti-CD19scFv, a CD8 stalk and transmembrane region, and an intracellular domain from CD3z. The first and second transcriptional units are separated by the b-globin polyA spacer A (SEQ ID NO:357) in the reverse orientation.

F1-3-637 encodes a bicistronic lentiviral genomic vector with divergent transcriptional units with the general structure shown in the schematic in FIG. 10. The first transcriptional unit encodes the lymphoproliferative element E006-T016-S186-S050 under the control of an NFAT-responsive minimal IL-2 promoter all encoded in the reverse orientation. The second transcriptional unit encodes CD19 CAR comprised of an anti-CD19scFv, a CD8 stalk and transmembrane region, and an intracellular domain from CD3z. The first and second transcriptional units are separated by the 250 cHS4 insulator (SEQ ID NO:358) in the forward orientation.

F1-3-748 encodes a bicistronic lentiviral genomic vector with divergent transcriptional units with the general structure shown in the schematic in FIG. 10. The first transcriptional unit encodes the lymphoproliferative element E016-T016-S186-S050 under the control of an NFAT-responsive minimal IL-2 promoter all encoded in the reverse orientation. The second transcriptional unit encodes a CD19 CAR comprised of an anti-CD19scFv, a CD8 stalk and transmembrane region, and an intracellular domain from CD3z followed by T2A and firefly luciferase. The first and second transcriptional units are separated by the 250 cHS4 insulator (SEQ ID NO:358) in the forward orientation.

F1-4-713 encodes a bicistronic lentiviral genomic vector with divergent transcriptional units with the general structure shown in the schematic in FIG. 10. The first transcriptional unit encodes the lymphoproliferative element E006-T016-S186-S050 under the control of an NFAT-responsive minimal IL-2 promoter all encoded in the reverse orientation. The second transcriptional unit encodes a CD22 CAR comprised of an anti-CD19scFv, a CD8 stalk and transmembrane region, and an intracellular domain from CD3z. The first and second transcriptional units are separated by the 250 cHS4 insulator (SEQ ID NO:358) in the forward orientation.

F1-5-221 encodes a membrane bound CD19 protein chimera comprised of the human CD19 extracellular domain the transmembrane domain and first 5 amino acids of the intracellular domain of the human PDGFR driver by the EF1-a promoter.

F1-6-744 encodes a bicistronic lentiviral genomic vector with divergent transcriptional units with the general structure shown in the schematic in FIG. 10. The first transcriptional unit encodes the lymphoproliferative element E016-T016-S186-S050 under the control of an NFAT-responsive minimal IL-2 promoter all encoded in the reverse orientation. The second transcriptional unit encodes a HER2 CAR comprised of an anti-HER2scFv, a CD8 stalk and transmembrane region, a CD137 intracellular domain, and an intracellular activating domain from CD3z followed by T2A and an eTag. The first and second transcriptional units are separated by the 250 cHS4 insulator (SEQ ID NO:358) in the forward orientation.

All mice used in the examples were handled in accordance with Institutional Animal Care and Use committee approved protocols.

### Example 2. Efficient Genetic Modification of Unstimulated Lymphocytes by Exposure of Whole Blood to Recombinant Retroviral Particles for 4 Hours Followed by Isolation of TNCS by Filtration

Unstimulated human T cells including NKT cells were effectively genetically modified by a 4 hour incubation of a reaction mixture that included whole blood and retroviral particles that were pseudotyped with VSV-G and displayed a T cell activation element on their surface. Total nucleated cells (TNCs) were subsequently captured from the transduction reaction mixture on a leukoreduction filter, washed, and collected by reverse perfusion of the leukoreduction filter assembly. The cell processing workflow was as shown in FIG. 1D with the exceptions that the optional steps of 170D and 180D were not performed, the final cells of step 160D were placed in culture, and only portions of the process were performed in a closed system. Transduction of CD3+ cells was assessed by expression of eTag using flow cytometry.

Viral supernatants were purified by a combination of depth filtration, TFF, benzonase treatment, diafiltration, and formulation, as described in Example 1, to generate the following substantially pure viral particles free of non-human animal proteins used in this Example: F1-3-23 pseudotyped with VSV-G and displaying the T cell activation element, UCHT1-scFvFc-GPI (F1-3-23GU).

Three 10 ml samples of whole fresh blood in Vacutainer tubes containing 16 USP units of Na-Heparin per mL of blood were purchased (StemExpress, San Diego) and combined in a 50 ml conical. Recombinant lentiviral particles F1-3-23GU (2.9 ml) were added directly to the 30 mL sample of whole blood at an MOI of 5 (assuming 1×10⁶ PBMCs/ml of blood) to initiate contacting of the lentiviral particles with lymphocytes in the whole blood, and incubated for 4 hours, at 37° C., 5% CO₂ with gentle mixing every hour to disrupt any sedimentation. After the 4 hour incubation, TNCs were isolated by processing the blood using a HemaTrate® Blood filtration System (Cook Regentec), a leukoreduction filter assembly, according to the manufacturer's instructions. The TNCs were then washed by passing 90 ml of DPBS +2% HSA over the leukoreduction filter assembly. TNCs were recovered into a flask by reperfusion with 20 ml X-Vivo15. TNCs were then cultured in a T75 flask at 37° C. and 5% CO₂. No exogenous cytokines were added to the samples at any time. Samples were collected at Day 7 to determine transduction efficiencies based on eTag and CD3 expression on live cells as determined by FACs analysis using a lymphocyte gate based on forward and side scatter.

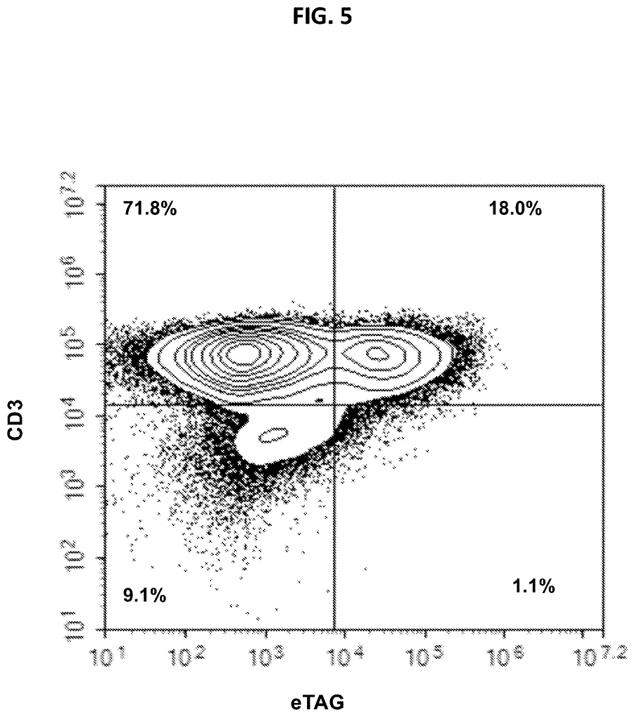

FIG. 5 shows a FACS profile of CD3+eTag+ cells at Day 7 after transduction of whole blood. Consistent with the surprising results in the previous Example, a 4 hour incubation of retroviral particles pseudotyped with VSV-G and displaying antiCD3-scFvFc with whole blood containing Na-Heparin was sufficient to effectively genetically modify the lymphocytes. Furthermore, a rapid TNC isolation step using a leukoreduction filter assembly was effective in isolating TNCs which include the transduced CD3+ T cells and NKT cells as evidenced by 17.99% of lymphocytes that stained positive for CD3 and eTag.

### Example 3. Subcutaneous Delivery of Modified PBMCs Significantly Enhanced CAR Cell Engraftment and Tumor Killing in Comparison to Intravenous Delivery

In this example, unstimulated PBMCs enriched from freshly isolated whole blood were modified using exemplary methods to express a CAR and an LE, and administered to mice within approximately 13 hours of the blood collection. The cell processing workflow was as shown in FIG. 1A with the exceptions that the optional step of 170A was not performed, and only steps 120A and 130A were performed in a closed system. Surprisingly, CAR cell engraftment and tumor killing in vivo was significantly enhanced by delivery of the modified PBMCs by subcutaneous injection as compared to intravenous injection. Materials and Methods

Recombinant lentiviral particles encoding F1-3-247 pseudotyped with VSV-G and displaying the T cell activation element, UCHT1-scFvFc-GPI (F1-3-247GU) were produced by transfecting F1XT cells using the 5 plasmid protocol at the 6.6 liter intermediate-scale and purified by a combination of depth filtration, TFF, benzonase treatment, diafiltration, and formulation to generate substantially pure viral particles free of non-human animal proteins as described in Example 1.

Whole blood from 2 healthy volunteers with informed consent was obtained and processed on separate days. Blood was collected into multiple 100 mm Vacutainer tubes (Becton Dickenson; 364606) containing 1.5 ml of Acid Citrate Dextrose Solution A anticoagulant (ACD peripheral blood). For each volunteer, blood from the Vacutainer tubes was pooled (204 ml for Donor A, 198 ml for Donor B) and distributed to 2 standard 500 ml blood collection bags.

To enrich for PBMCs, blood in the 2 blood bags from each volunteer was processed sequentially in a closed system by density gradient centrifugation with Ficoll-Paque™ (General Electric) using a CS-900.2 kit (BioSafe; 1008) on a Sepax 2 S-100 device (Biosafe; 14000) using 2 wash cycles according to the manufacturer's instructions, to obtain 45 ml of isolated PBMCs from each run. The wash solution used in the Sepax 2 process was Normal Saline (Chenixin Pharm)+2% human serum albumin (HSA) (Sichuan Yuanda Shuyang Pharmaceutical). The final cell resuspension solution was 45 ml Complete OpTmizer™ CTS™ T-Cell Expansion SFM (OpTmizer™ CTS™ T-Cell Expansion Basal Medium 1 L (Thermo Fisher, A10221-03) supplemented with 26 ml OpTmizer™ CTS™ T-Cell Expansion Supplement (Thermo Fisher, A10484-02), 25 ml CTSTM Immune Cell SR (Thermo Fisher, A2596101), and 10 ml CTS™ GlutaMAX™-I Supplement (Thermo Fisher, A1286001)). Each processing step on the Sepax 2 machine was approximately 1 hour and 20 minutes. 3×10⁸ live PBMCs were obtained from Donor A and 1.6×10⁸ live PBMCs were obtained from Donor B.

For transduction, freshly enriched PBMCs were seeded in 50 ml tubes and Complete OpTmizer™ CTS™ T-Cell Expansion SFM was added to bring the cell density to 1.0×10⁶ cells/ml. No anti-CD3, anti-CD28, IL-2, IL-7, or other exogenous cytokine was added to activate or otherwise stimulate the PBMCs ex vivo prior to transduction. F1-3-247GU viral particles were added to the non-stimulated PBMCs at an MOI of either 1 or 5 depending upon the sample. The transduction reaction mixtures were incubated for four (4) hours in a standard humidified tissue culture incubator at 37° C. and 5% CO₂. After the 4 hour exposure, the cells were pelleted for 10 minutes at 400 g and washed 3 times by resuspending the cells in 40 ml of DPBS +2% HSA and centrifuging for 10 minutes at 400 g, before being resuspended in 5 ml DPBS +2% HSA and counted.

As a control for the in vivo studies, transduction efficiencies were determined by in vitro assays. 1.0×10⁶ cells of each transduction were seeded in wells of a 24-well tissue culture plate in 1 ml of Complete OpTmizer™ CTS™ T-Cell Expansion SFM and incubated in a standard humidified tissue culture incubator at 37° C. and 5% CO₂. No exogenous cytokines were added to the samples at any time. Samples were collected at Day 6 to determine transduction efficiencies based on eTAG and CD3 expression as determined by FACs analysis using a lymphocyte gate based on forward and side scatter.

For the in vivo studies, samples of the transduced (or otherwise modified) PBMCs were resuspended at 1.0×10⁶ and 5.0×10⁶ PBMCs per 200 μl DPBS+2% HSA for dosing. The total elapsed time to collect blood, enrich for PBMCs, transduce or otherwise modify the PBMCs, and prepare the PBMCs for dosing was 12 hours forty minutes for Donor A and 13 hours for Donor B.

### Proliferation/Survival and Target Killing of Tumors In Vivo by Effector PBMCs Transduced by the Methods Above

A xenograft model using B-NDG mice was chosen to probe the ability of human PBMCs transduced with F1-3-247 to survive, proliferate, and kill CD19-expressing tumors in vivo. B-NDG is a strain of mice that lack mature T cells, NK cells, and B cells and is among the most immunodeficient mouse strain described to date. Removal of these cellular components of the immune system is typically performed to enable human PBMCs to engraft without innate, humoral, or adaptive immune reactions from the host. Concentrations of homeostatic cytokines normally present only after radiation or lymphodepleting chemotherapy in humans is achieved due to the absence of the murine extracellular common gamma chain, which enables adoptively transferred human cells to receive such cytokines. At the same time, these animals can also be utilized to engraft tumor xenograft targets to examine the efficacy of CARs to kill target-expressing tumors. While the presence of xenoreactive T cell receptor antigens in the effector cellular product will eventually give rise to graft versus host disease, these models enable short term evaluation of animal pharmacology and acute tolerability.

Raji cells (ATCC, Manassas, Va.) which express endogenous human CD19 were utilized to provide antigen to stimulate the CAR effector cells and to generate uniform target tumors to determine the efficacy of CAR effector cells to kill CD19-expressing tumors. The Raji cells grew rapidly with subcutaneous administration into NSG mice in combination with Matrigel artificial basement membrane.

Subcutaneous (sc) tumor xenografts were established in the hind flank of female NOD-Prkdc^{scid}Il2rg^{tm1}/Bcgen (B-NDG) mice (Beijing Biocytogen Co. Ltd.). Briefly, cultured Raji cells were washed in DPBS (Thermo Fisher), counted, resuspended in cold DPBS and mixed with an appropriate volume of Matrigel ECM (Corning; final concentration 5 mg/ml) at a concentration of 0.5×10⁶ cells/200 μl Matrigel on ice. Animals were prepared for injection using standard approved anesthesia with hair removal (Nair) prior to injection. 200 μl of cell suspension in ECM was injected subcutaneously into the rear flanks of 6 week old mice.

Modified PBMCs from Donor A were delivered to mice intravenously. 14 days after tumor inoculation, mice bearing Raji tumors, which averaged 150 mm³ in volume, were dosed intravenously with 200 μl of PBMCs from Donor A by tail vein injection as follows: AG1 received 1×10⁶ untransduced PBMCs (n=5), AG2 received 1×10⁶ PBMCs transduced with F1-3-247GU at an MOI of 1 (n=6), AG3 received 5×10⁶ PBMCs transduced with F1-3-247GU at an MOI of 1 (n=6), AG4 received 1×10⁶ PBMCs transduced with F1-3-247GU at an MOI of 5 (n=6), and AG5 received 5×10⁶ PBMCs transduced with F1-3-247GU at an MOI of 5 (n=6).

Modified PBMCs from Donor B were delivered to mice subcutaneously rather than intravenously. 18 days after tumor inoculation, mice bearing Raji tumors, which averaged 148 mm³ in volume, were dosed subcutaneously in the opposite flank from the tumor with 100 μl of PBMCs from Donor B as follows: BG1 received 5×10⁶ untransduced PBMCs (n=5), BG2 received 5×10⁶ PBMCs transduced with F1-3-247GU at an MOI of 1 (n=5), BG3 received 1×10⁶ PBMCs transduced with F1-3-247GU at an MOI of 5 (n=6), and BG4 received 5×10⁶ PBMCs transduced with F1-3-247GU at an MOI of 5 (n=6).

Tumors were measured using calipers 2 or 3 times a week and tumor volume was calculated using the following equation: (longest diameter*shortest diameter²)/2. Approximately 100 μl of blood was collected from each mouse on days 7 (or 8), 14, 21, 28, and 35 for analysis by FACS and qPCR.

### RESULTS

Whole human blood was collected from 2 healthy volunteers and enriched for PBMCs by Ficoll-Paque™ on a Sepax 2 S-100 device. FACs analysis was used to characterize the cellular composition of the enriched PBMCs which were subsequently transduced and delivered in vivo to mice. Table 2 shows the percentage of cells expressing select markers. Note that in addition to T and NK cells, these enriched PBMCs included 6.9% and 21.9% CD14+ cells (macrophage, dendritic cells, and neutrophils) from Donors A and B, respectively, and 1.9% and 9.8% CD19+ cells (B cells) from Donors A and B, respectively.

TABLE 2
Percentage of freshly enriched PBMCs expressing select markers.

| Population | Markers | Donor A | Donor B |
|---|---|---|---|
| % Live Lymphocytes | CD3+ | 77.30% | 37.20% |
| | CD3−CD56+ | 4.90% | 23% |
| % CD3+ Live | CD3+CD4+ (in CD3+) | 49.30% | 42.30% |
| Lymphocytes | CD3+CD8+ (in CD3+) | 58.80% | 52.50% |
| | CD3+CD56+ (in CD3+) | 6.40% | 16.40% |
| % Live Cells | CD14+ | 6.90% | 21.90% |
| | CD19+ | 1.90% | 9.80% |

The enriched PBMCs were genetically modified with F1-3-247GU to express a CAR to CD19 and a lymphoproliferative element comprising the parts E006-T016-S186-S05 (Table 1) driven constitutively by the EF1-α promoter. To genetically modify the PBMCs, the cells were incubated for 4 hours with lentiviral particles encoding F1-3-247 that were pseudotyped with VSV-G and that also displayed UCHT1-scFvFc-GPI on their surface. A sample of each transduction reaction was cultured in vitro for 6 days in the absence of exogenous cytokines and transduction efficiencies were determined as the percentage of CD3+eTAG+live cells using flow cytometry. Transduction efficiencies of PBMCs from Donor A were 4.5% and 51.2% at MOIs of 1 and 5, respectively. Transduction efficiencies of PBMCs from Donor B were 15.7% and 24.8% at MOIs of 1 and 5, respectively. Consistent with the previous examples, these results demonstrate that the PBMCs were effectively transduced.

For the in vivo arms of this example, B-NDG Immunodeficient mice bearing CD19 tumors were dosed with PBMCs that had been modified through a 4 hour exposure to F1-3-247GU. These PBMCs were never expanded or otherwise cultured ex vivo prior to dosing. Rather, the modified PBMCs were used to dose the mice within 13 hours of being collected as whole blood from volunteers. Modified PBMCs from Donor A were dosed traditionally by intravenous administration, while modified PBMCs from Donor B were dosed subcutaneously in the flank opposite to the tumor.

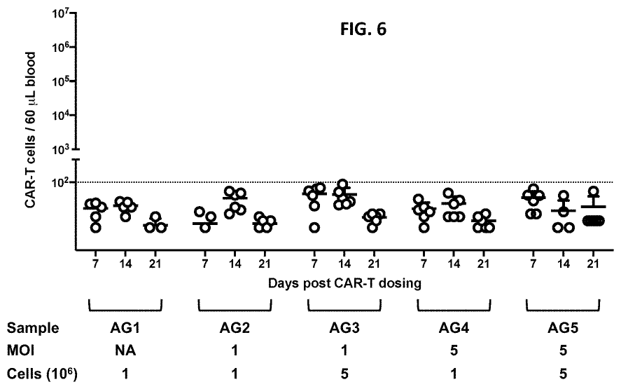

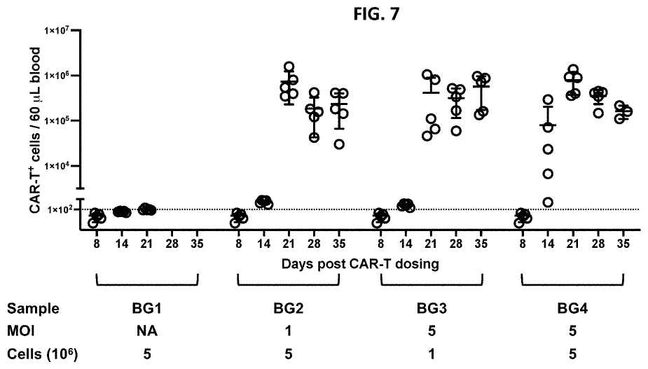

The ability of these transduced PBMCs to engraft in vivo were examined once a week for up to five weeks after CAR-T dosing. FIGS. 6 and 7 show the number of CAR-T cells per 60 μl of blood as detected by flow cytometry for CD3+eTAG+cells. As shown in FIG. 6, when compared to untransduced PBMCs (AG1) PBMCs transduced with F1-3-247GU and delivered intravenously did not exhibit appreciable engraftment even when the transduction was performed at an MOI of 5 and 5×10⁶ cells were delivered (AG5). In contrast, as shown in FIG. 7, significant engraftment was observed in all mice when the PBMCs transduced with F1-3-247GU were delivered subcutaneously. At 21 days post CAR-T dosing, for example, the average number of CAR-T cells per 60 μl of blood was only 103 in mice that received untransduced PBMCs (BG1), but was 7.3×10⁵, 4.2×10⁵, and 7.9×10⁵ CAR-T cells/60 μl of blood in BG2, BG3, and BG4, respectively, that each received transduced PBMCs.

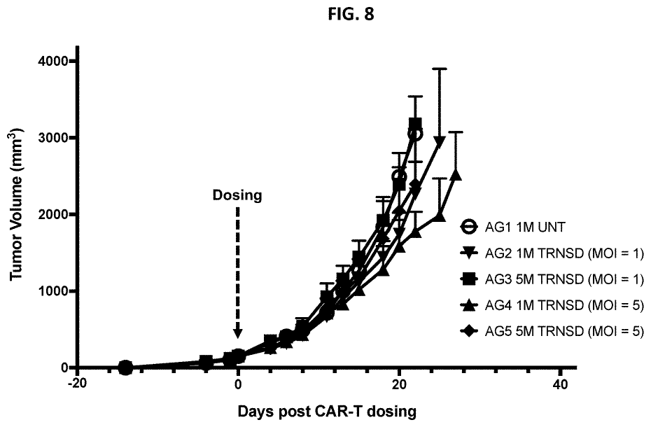

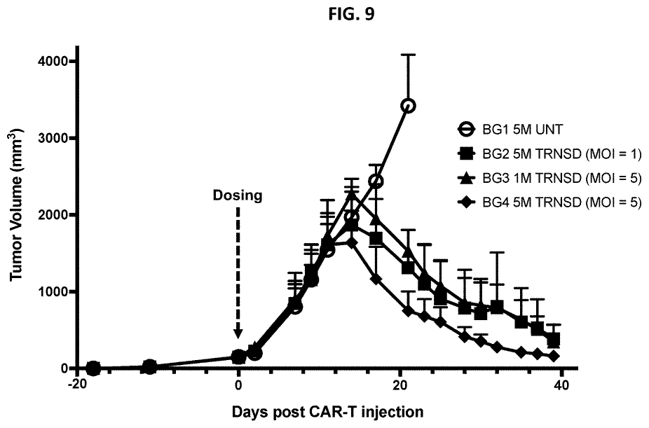

The ability of these transduced PBMCs to kill established Raji tumors in vivo was examined over time. As shown in FIG. 8, PBMCs transduced with F1-3-247GU and delivered intravenously can exhibit a modest ability to inhibit tumor progression. This is seen in samples AG2, AG4, and AG5. In contrast, as shown in FIG. 9, PBMCs transduced with F1-3-247GU and delivered subcutaneously led to a dramatic reduction in tumor burden. This tumor regression was observed in all mice in groups BG2, BG3, and BG4.

Together these results demonstrate that PBMCs isolated, manipulated ex vivo to express a CAR and a lymphoproliferative element, and delivered in vivo within 13 hours of the initial blood draw, can engraft in vivo and promote tumor regression. Surprisingly, subcutaneous delivery of the modified PBMCs led to significantly better engraftment and tumor regression as compared to intravenous delivery.

### Example 4. Transduction of Activated PBMCs with Recombinant Retroviral Particles Encoding Bicistronic Lentiviral Genomic Vectors to Generate Self-Driving Cars

In this Example, PBMCs were transduced with two representative bicistronic vectors (F1-3-635 and F1-3-637) from Example 3 and compared with two monocistronic vectors (F1-3-23 and F1-3-247). The transduced PBMCs were stimulated repeatedly over time with Raji cells which express CD19 targets for the CD19 CAR. This stimulation resulted in induced expression of the lymphoproliferative element and expansion of the transduced cells.

The constructs used in this Example were F1-3-23, F1-3-247, F1-3-635, and F1-3-637. Recombinant lentiviral particles were produced by transient transfection of 30 ml of F1XT using a 4 vector packaging system and purified by PEG precipitation as described in Example 1. Each sample was resuspended in 0.3 ml PBS with 3 mg/ml HSA.

On Day 0, PBMCs from a single donor were enriched from buffy coats (San Diego Blood Bank) by density gradient centrifugation with Ficoll-Paque PREMIUM® (GE Healthcare Life Sciences) according to the manufacturer's instructions followed by lysis of red blood cells. 1.5×10⁶ viable PBMCs were seeded in the wells of G-Rex 6 Well Plates (Wilson Wolf, 80240M) in 3 ml Complete OpTmizerTM CTS™ T-Cell Expansion SFM supplemented with 100 IU/ml (IL-2), 10 ng/ml IL-7, and 50 ng/ml anti-CD3 antibody (317326, Biolegend) to activate the PBMCs for viral transduction. After incubation overnight at 37° C. and 5% CO₂, lentiviral particles including the constructs described above were added directly to the activated PBMCs at an MOI of 5 and incubated overnight at 37° C. and 5% CO₂. The following day, the media volume in each well was brought to 30 ml with Complete OpTmizer™ CTS™ T-Cell Expansion SFM and the plates were returned to the incubator.

The cells from each well were collected on Day 7, washed, and reseeded in the wells of G-Rex 24 Well Plates at 0.5×10⁶ cells in 1 ml of Complete OpTmizerTM CTS™ T-Cell Expansion SFM. 1×10⁶ Raji, which express CD19 that is recognized by the CD19 CAR, were added to samples designated as “fed” or no Raji cells were added to the samples designated as “unfed.” The volume in each well was brought up to 7 ml with Complete OpTmizer™ CTS™ T-Cell Expansion SFM. No IL-2, IL-7, or other exogenous cytokine was added at this or subsequent cell culture steps. Raji cells were added to the fed transduced PBMC samples every other day until Day 15 by removing 3 ml of media and replacing it with fresh media containing 1×10⁶ Raji cells. The cell density of transduced PBMCs was very high on Day 15 so the feeding protocol was modified. Starting on Day 15, 1.0×10⁶ CAR+ cells were reseeded into wells of new G-Rex 24 Well Plates, 1×10⁶ Raji cells were added, and the volume was brought to 7 ml with Complete OpTmizerTM CTS™ T-Cell Expansion Media.

To analyze the expansion of CAR+ T and NK cells, 100 ul of cells were removed at each time point and stained for the expression of CD3, eTag, and CD19 CAR. Flow cytometry was used to count the total live cells, and the percent of cells expressing CD3, eTag, and CD19 CAR. Total CD3+CAR+ cells were calculated by multiplying the total live cells in the lymphocyte gate by the percentage of CD3+CAR+ cells. eTAG% was determined from within the live CD3+CAR+ population.

### RESULTS

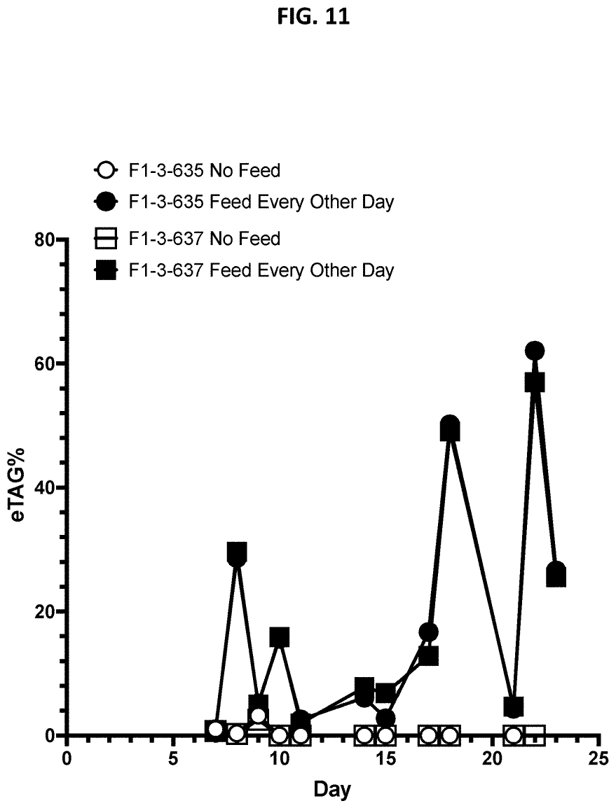

In this Example, activated PBMCs were transduced with viral particles containing a bicistronic lentiviral genomic vector that encoded a first transcriptional unit comprising a eTagged lymphoproliferative element, E006-T016-S186-S050, under the control of an NFAT-responsive minimal IL-2 promoter in the reverse orientation followed by an insulator and a second transcriptional unit encoding a first generation CD19 CAR under the control of the EF1-a promoter in the forward orientation. These transduced PBMCs were then stimulated with cells expressing the CAR target, in this case CD19-expressing Raji cells, every other day (herein referred to as “feeding”) or left unfed. As shown in FIG. 11, activation of the CAR expressed from the second transcriptional unit led to the induced expression of the eTagged lymphoproliferative element from the second transcriptional unit. In this Example, the percentage of cells that expressed eTag increased at 24 hours post stimulation then decreased to near the original percentage by 48 hours post stimulation, at which time the cells were stimulated again by feeding. This pattern repeated for each of the six feedings.

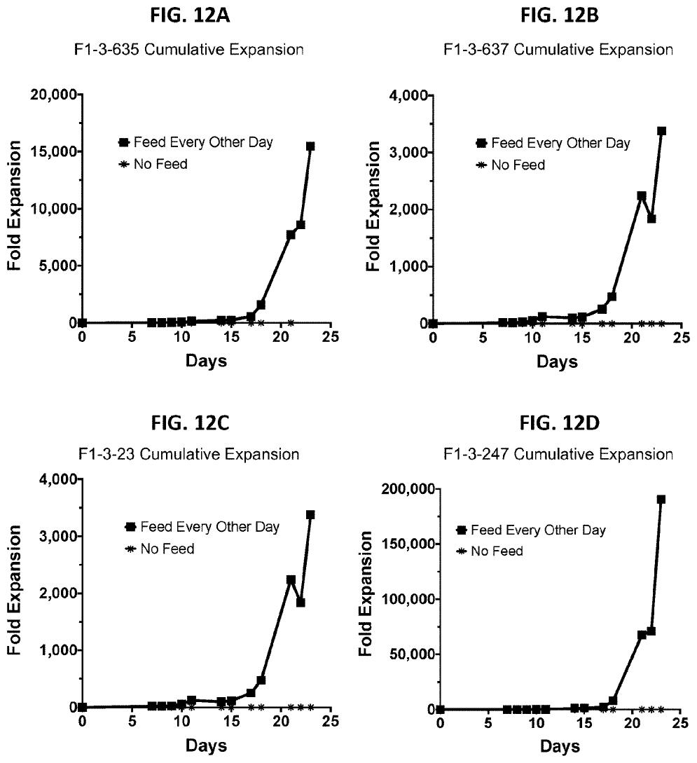

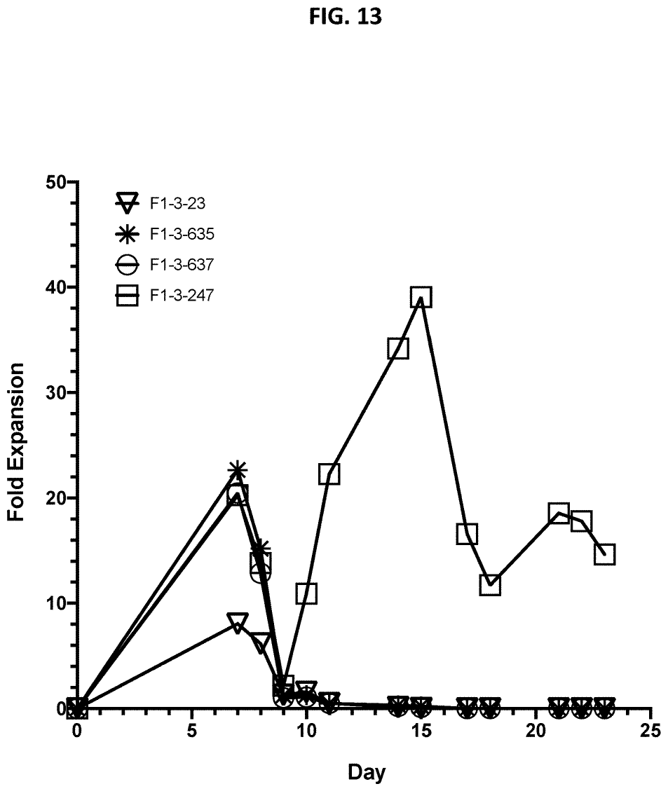

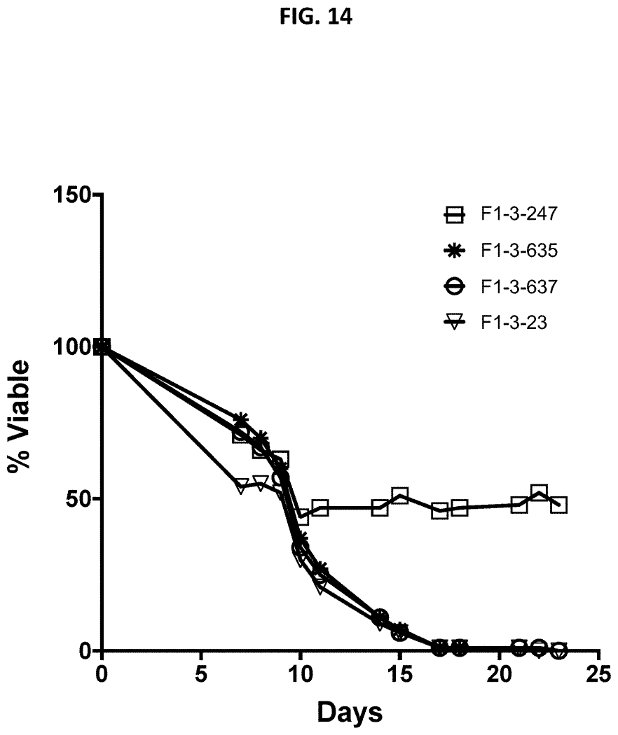

Activation of the constitutively expressed CAR by feeding every other day led to induced expression of the eTagged lymphoproliferative element which then resulted in proliferation of the CD3+CAR+ cells. PBMCs transduced with F1-3-635 expanded over 15,000-fold over 23 days as shown in FIG. 12A. PBMCs transduced with F1-3-637 expanded over 3,000-fold over 23 days as shown in FIG. 12B. In contrast, PBMCs transduced with F1-3-23 which has a CD19 CAR but lacks the lymphoproliferative element, expanded less than 40-fold by day 23 as shown in FIG. 12C. PBMCs transduced with F1-3-247, which expressed the lymphoproliferative element constitutively, expanded 190,000-fold as shown in FIG. 12D. It is noteworthy that the most expansion by PBMCs transduced with F1-3-635, F1-3-637, and F1-3-247 occurred during the 8 days between day 15 and day 23. This is likely because the cells were at a high density prior to Day 15 and reseeding the cells at 1.0×10⁶ CAR+ cells per well at each subsequent feeding allowed them room to expand. In contrast, in the absence of the addition of cytokines and CAR activation by feeding, expression of the lymphoproliferative element was not induced in PBMCs transduced with F1-3-635 or F1-3-637, and the expansion (shown in FIG. 13) and percent viability (shown in FIG. 14) of these cells was no greater than PBMCs transduced with F1-3-23. PBMCs transduced with F1-3-247, however, which expressed the lymphoproliferative element constitutively, did expand to a greater extent than cells transduced with F1-3-23, and the viability remained at approximately 50% from Day 10 to Day 23. In the unfed samples, PBMCs transduced with F1-3-635 or F1-3-637 showed an initial expansion and percent viability similar to PBMCs transduced with F1-3-247 prior to Day 9. This effect could be due to transcription from the NFAT-responsive promoter caused by the activation of the PBMCs with anti-CD3 antibody, which activates NFAT through CD3z.

This Example demonstrates that viral particles comprising bicistronic lentiviral genomic vectors with divergent transcriptional units comprising a first transcriptional unit encoding a lymphoproliferative element under transcriptional control of a CAR-stimulated inducible promoter and a second transcriptional unit encoding a CAR under transcriptional control of a constitutive T cell or NK cell promoter, can be used to transduce lymphocytes to generate self-driving CAR T cells that proliferate and survive only in the presence of antigen. Therefore, self-driving CAR T cells will mount an immune response against antigen-expressing cells, and the immune response will resolve when the self-driving CAR T cells eliminate and run out of antigen-expressing cells to stimulate the CAR T cells.

### Example 5. Self-Driving Cars Manufactured by Exposure of Whole Blood to Lentiviral Particles Encoding Bicistronic Genomic Vectors for 4 Hours Followed by a PBMC Enrichment Procedure and Administered Subcutaneously Show Efficacy Against Systemic Human Burkitt's Lymphoma in a Murine Model

In this example, unstimulated human T and NKT cells were genetically modified by an rPOC cell processing method using replication incompetent recombinant (RIR) retroviral particles encoding bicistronic genomic vectors to generate self-driving CAR cells expressing a CAR directed to CD19 or CD22, and a lymphoproliferative element. The cell processing workflow was performed as shown in FIG. 1C with the exception that the optional step of 170C was not performed and not all steps were performed in a closed sysetm. Self-driving PBMCs were injected subcutaneously into NSG MHC I/II knockout mice with systemic Raji-luc tumors. Mice were assessed for tumor burden and survival.

Recombinant lentiviral particles used in this example comprised either F1-3-637 or F1-4-713 bicistronic lentiviral genomic vectors. F1-3-637 was described in Example 4. Both constructs were identical except for the CAR's ASTR which is directed to CD19 and CD22 for F1-3-637 and F1-4-713, respectively. Both retroviral particles were pseudotyped with VSV-G, displayed the T cell activation element UCHT1-scFvFc-GPI, and were produced by transfecting F1XT cells using the 5 plasmid protocol at the 10 liter intermediate-scale as described in Example 1. Viral supernatants were purified by a combination of depth filtration, TFF, benzonase treatment, diafiltration, and formulation to generate substantially pure viral particles (F1-3-637GU and F1-4-713GU) free of non-human animal proteins.

Whole blood from a healthy volunteer with informed consent was collected into tubes containing heparin. 75 ml was transferred into each of 2 blood bags. No blood cell fractionation or enrichment was performed before the whole blood was contacted with retroviral particles. 3.75×10⁸ TU of F1-3-637GU (7.31 ml) was added to one blood bag, and 3.75×10⁸ TU of F1-3-713GU (13.07 ml) was added to the other bag such that virus was added at an MOI of 5 based on the assumption that there were 1.0×10⁶ CD3+ cells/ml of blood. The bags were inverted 5 times to mix the contents, then incubated for 4 hours, at 37° C., 5% CO₂. Following the 4 hour contacting time, PBMCs were enriched density gradient centrifugation with Ficoll-Paque™ (General Electric) using a CS-900.2 kit (BioSafe; 1008) on a Sepax 2 S-100 device (Biosafe; 14000) using 2 wash cycles according to the manufacturer's instructions, to obtain 45 ml of isolated PBMCs from each run. The wash and final resuspension solution used in the Sepax 2 process was Normal Saline (Chenixin Pharm)+2% human serum albumin (HSA) (Sichuan Yuanda Shuyang Pharmaceutical). The cells were counted, and 7.5×10⁷ cells from each transduction was pelleted for 5 minutes at 400 g and resuspended at 2.5×10⁷ cells/ml in 3 ml normal saline +2% HSA.

The ability of anti-CD19, anti-CD22, and a combination of both anti-CD19 and anti-CD22 self-driving CARs to treat a model of systemic Human Burkitt's Lymphoma was examined in a mouse model. Female NSG-(KbDb)null (IA)null (MHC I & II double knockout) mice were used in this study. Each mouse was inoculated with 3.0×10⁵ Raji-Luciferase cells in 100 μl of PBS via intravenous tail vein injection for tumor development on day −4. Raji cells naturally express both CD19 and CD22. 25 mice were randomly allocated into 5 groups (5 mice/group) for administration of test articles in 200 μl PBS subcutaneously. Mice in each group received the following test articles on Day 0: G1, PBS; G2, 5.0×10⁶ untransduced PBMCs; G3, 5.0×10⁶ PBMCs transduced with F1-3-637GU; G4, 5.0×10⁶ transdcued with F1-4-713; and G5, 2.5×10⁶ PBMCs transduced with F1-3-637GU and 2.5×10⁶ PBMCs transduced with F1-4-713GU.

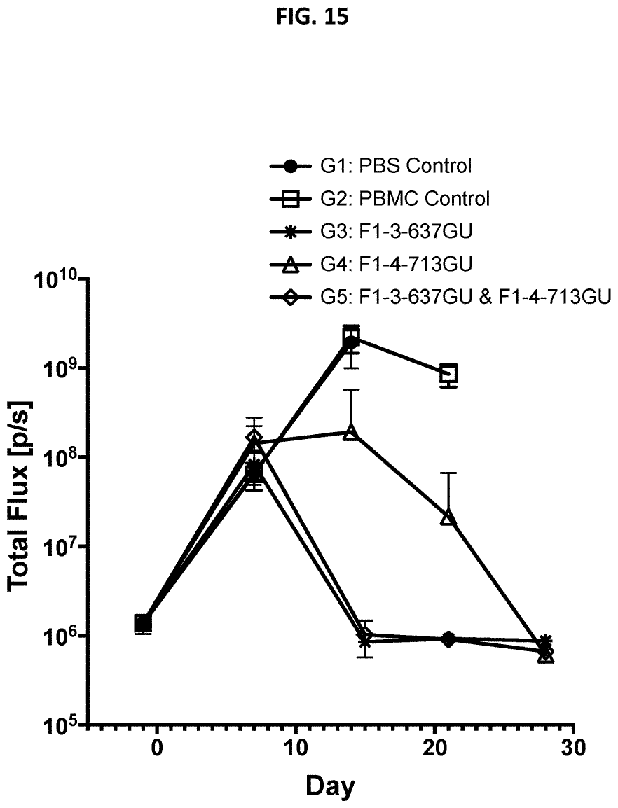

Mice were assessed for tumor growth by bioluminescent imaging (PerkinElmer, IVIS Lumina Series II) and analyzed with LivingImage software. As shown in FIG. 15, PBMCs transduced with F1-3-637GU alone or PBMCs transduced with F1-3-637GU in combination with PBMCs transduced with F1-4-713GU resolved systemic Raji tumors by Day 15 post subcutaneous delivery of these self-driving CARs Similarly, PBMCs transduced with F1-3-637GU alone resolved systemic Raji tumors by Day 28. In contrast, mice that received untransdcued PBMCs or PBS and that were still alive, had substantial tumor burden on Days 14 thru Day 28 as indicated by a total average flux of greater than 10⁸ p/s.

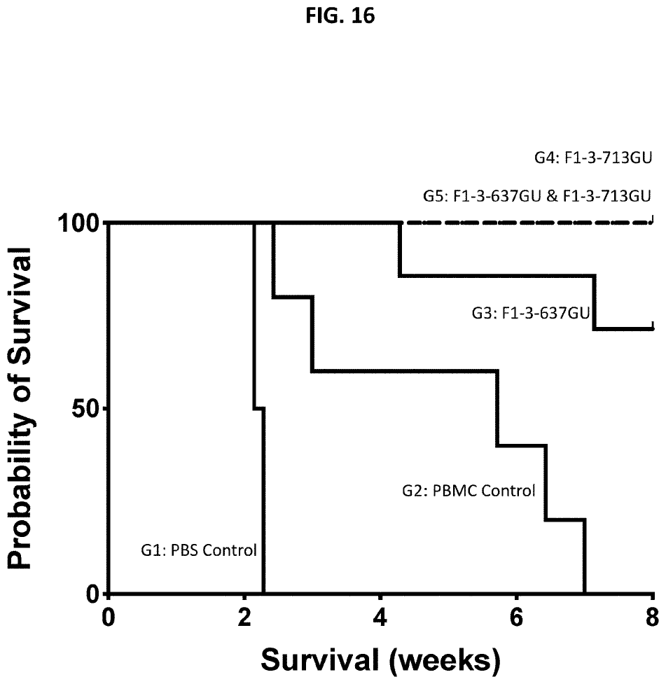

Survival analysis is shown in FIG. 16. All 5 mice in G4 and G5 survived for 8 weeks. From G3, one mouse was found dead on Day 30 and another on Day 50, both after the tumor burden had been resolved on Day 15 with histologic signs of GVHD. In contrast, none of the mice from G2 and G1 survived past Day 49 and Day 16, respectively.

This example demonstrates that lentiviral particles encoding bicistronic genomic vectors and displaying the activation element UCHT1-scFvFc-GPI on their surface, when incubated with whole blood for 4 hours, can transduce PBMCs. When delivered subcutaneously, these transduced PBMCs, which were self driving CARs expressing a lymphoproliferative element and a CAR directed to either CD19 or CD22 were capable of expanding in vivo and eliminating systemic Raji tumors. This ability to clear systemic Raji tumors was observed when self-driving CARs directed to CD19 alone, CD20 alone, or a combination of CARs directed to both CD19 and CD22 were delivered to the mice.

### Example 6. Genetic Modification of Unstimulated Lymphocytes by Exposure of TNCs on a Leukoreduction Filter to Recombinant Retroviral Particles for 4 Hours

In this example, the genetic modification of lymphocytes by 2 different cell processing workflows that include capturing TNCs, were compared side-by-side. The first cell processing workflow (“1D”) was as described in Example 2 and as shown in FIG. 1D, with the exceptions that the optional steps of 170D and 180D were not performed, the final cells of step 160D were placed in culture, and only portions of the process were performed in a closed system. In this first process, unstimulated human T cells and NKT cells were effectively genetically modified by a 4 hour incubation of a reaction mixture at 37° C., 5% CO₂ that included whole blood and retroviral particles that were pseudotyped with VSV-G and displayed a T cell activation element on their surface. Total nucleated cells (TNCs) were subsequently captured from the transduction reaction mixture on a leukoreduction filter, washed, and collected by reverse perfusion of the leukodepletion filter assembly. The second cell processing workflow (“1B”) was as shown in FIG. 1B with the exceptions that the optional steps of 170B and 180B were not performed, the final cells of step 160B were placed in culture, and only portions of the process were performed in a closed system. In this process, whole blood was passed through a leukoreduction filter to capture TNCs and unstimulated human T cells and NKT cells were effectively genetically modified by a 4 hour incubation of a reaction mixture on the filter that included TNCs and the same retroviral particles used in the first cell process. After 4 hours on the filter, the cells were washed and collected by reverse perfusion of the leukodepletion filter assembly. In each case, the transduced TNCs were placed in culture with rIL-2. Transduction of CD3+ cells was assessed on Day 6 by expression of the CAR polypeptide using flow cytometry. CAR-T function was testing by IFN gamma production on Day 7.

Recombinant lentiviral particles encoding F1-3-637 (described in Example 4.) pseudotyped with VSV-G and displaying the T cell activation element, UCHT1-scFvFc-GPI (F1-3-637GU) were produced by transfecting F1XT cells using the 5 plasmid protocol at the 10 liter intermediate-scale. Viral supernatants were purified by a combination of depth filtration, TFF, benzonase treatment, diafiltration, and formulation as described in Example 1, to generate substantially pure F1-3-637GU viral particles free of non-human animal proteins.

For cell processing workflow 1D, 12 ml of heparinized whole blood from a healthy human donor was transferred to a blood bag (CS50, Origen). 1.17 ml of recombinant lentiviral particles F1-3-637GU (5.13×10⁷ TU/ml) were added directly to the 12 mL sample of whole blood at an MOI of 5 (assuming 1×10⁶ PBMCs/ml of blood) to initiate contacting of the lentiviral particles with lymphocytes in the whole blood, and incubated for 4 hours, at 37° C., 5% CO₂ with gentle mixing every hour to disrupt any sedimentation. After the 4 hour incubation, TNCs were isolated by processing the blood through an Acrodisc® leukodepletion filter. The TNCs were then washed by passing 50 ml of NS-HSA2%-heparin50U/ml over the leukoreduction filter (AP-4952, Pall) assembly. TNCs were recovered into a 20 ml syringe by reperfusion with 8 ml NS-HSA2%, centrifuged for 5 min at 400g, and resuspended in Complete OpTmizer™ CTS™ T-Cell Expansion SFM (“CTS media”). 3×10⁶ cells were in cultured in 3 ml of CTS media with 10 ng/ml rhlL-2 per well. 23 ml additional CTS media and 10 ng/ml rhIL-2 were added on Days 2 and 4.

For cell processing workflow 1B, 12 ml of heparinized whole blood from a healthy human donor was transferred to a blood bag. TNCs were isolated by processing the blood through an Acrodisc®. The TNCs were then washed three times by passing 10 ml of NS-HSA2%-heparin50U/ml over the leukoreduction filter. 1.17 ml of recombinant lentiviral particles F1-3-637GU (5.13×10⁷ TU/ml) was mixed with 650 μl HSA and 780 μl CTS media and 650 μl of this virus solution, which was maintained at 37° C., was added to the filter at 0, 1, 2, and 3 hours. The leukodepletion filter with the transduction mixture was incubated at 37° C., 5% CO₂ for 4 hours. The TNCs were then washed by passing 50 ml of NS-HSA2%-heparin50U/ml over the Acrodisc®. TNCs were recovered into a 20 ml syringe by reperfusion with 8 ml NS-HSA2%, centrifuged for 5 min at 400 g, resuspended in CTS media and counted (Day 0). 1.5×10⁶ viable TNCs were seeded in the wells of G-Rex 6 Well Plates (Wilson Wolf, 80240M) in 3 ml CTS media supplemented with 10 ng/ml rhlL-2.

Cells in some of the wells were harvested on Day 6, and analyzed for transduction efficiency and cell surface markers by flow cytometry. For analysis of CAR-T cell functionality by IFNgamma release, on Day 6, cells were left untreated or were treated with PMA (100 mM)+Ionomycin (1 μg/ml), CHO-S or Raji target cells at a ratio of 5:1 PBMC:target, and incubated at 37° C., 5% CO₂. After 16 hours, cell culture supernatants were harvested and analyzed by ELISA for IFNgamma

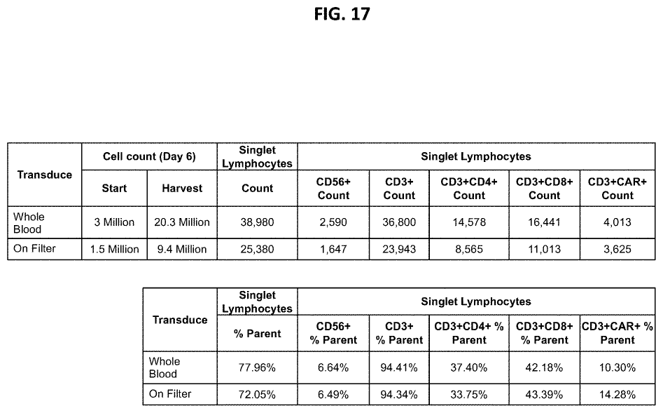

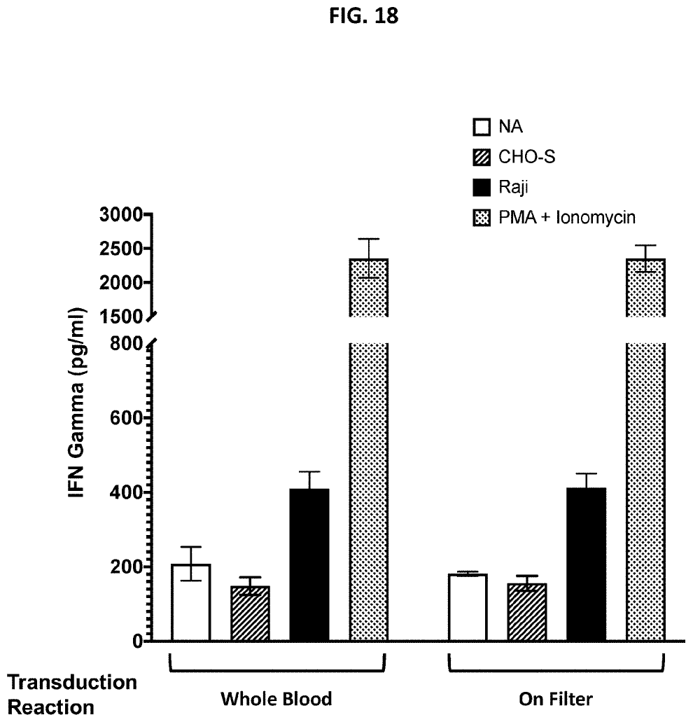

Both cell processes started with 12 ml of heparinized whole blood from the same donor. Recovery of live TNCs off the leukoreduction filter on Day 0 was 10.3×10⁶ cells for process 1B and 5.0×10⁶ cells for process 1D. These results suggest that performing the transduction reaction for 4 hours at 37° C., 5% CO₂ leads to adherence of TNCs to the filter. This adherence impedes recovery and led to the development of alternative processes such as those that include shorter incubation periods, reduced temperatures, and/or eluting the cells off the leukoreduction filter prior to the contacting step (as described in FIG. 1E and FIG. 1F). Cell surface marker expression of the harvested TNCs after 6 days of culture in CTS media supplemented with rhIL-2 is shown in FIG. 17. The percentages of CD56+ cells, CD3+CD4+, and CD3+CD8+ cells were roughly equivalent in the cells processed by methods 1B and 1D. The percentage of transduced T cells as determined by CD3 and CAR expression was 10.30% for transduction in whole blood (1B) and 14.28 for transduction on the filter (1D). This represents a 38% improvement in transduction efficiency when the cells are transduced while concentrated on the filter. FIG. 18 shows that TNCs transduced by either process 1B or 1D responded to stimulation with Raji cells (which express CD19 target of the antiCD19 CAR encoded by F1-3-637) or PMA by secreting IFNgamma to a similar extent, and this level was above background indicating that the T cells transduced by F1-3-637GU retroviral particles by these methods are functional.

These results show that the cell processing workflows shown in FIG. B1 and FIG. D1 are viable rPOC workflows for cell therapy. While performing the transduction reaction on concentrated cells on the leukoreduction filter at 37° C. for 4 hours may lead to an increased transduction efficiency, the recovery of cells off the filter is impeded by adherence of cells to the filter. Not to be bound by theory, it is believed that these adherent cells are T cells that were activated and as a result, expressed adhesion molecules. It is believed that a high percentage of these cells were also transduced. Therefore, improvements to the process include methods to inhibit the adherence of cells to the filter, such as reducing the time and/or temperature of the incubation, and modifying the wash and/or delivery solution to promote the release of cells bound to the filter.

### Example 7. Self-Driving Cars Manufactured by Exposure of Whole Blood to Lentiviral Particles Encoding Bicistronic Genomic Vectors for 4 Hours Followed by Either a TNC Enrichment Procedure or a PBMC Enrichment Procedure, When Administered Subcutaneously, Can Eliminate Systemic Human Burkitt's Lymphoma in a Murine Model

In this example, unstimulated human T and NKT cells freshly drawn from peripheral blood were genetically modified by an rPOC cell processing method from heparinized whole blood using replication incompetent recombinant (RIR) retroviral particles encoding bicistronic genomic vectors to generate self-driving CAR cells expressing a CAR directed to CD19 and a lymphoproliferative element to compare the effect of inoculating purified PBMCs versus TNCs. The cell processing workflows were performed as shown in FIG. 1C and FIG. 1D with the exceptions that the optional steps of 170C, 170D and 180D were not performed, and not all steps were performed in a fully closed sysetm. Modified PBMCs or TNC or controls were injected subcutaneously into NSG mice bearing systemic Raji-luc tumors. Mice were assessed for tumor burden and survival.

Recombinant lentiviral particles used in this example comprised the F1-3-637 bicistronic lentiviral genomic vector. F1-3-637 is described in Example 4. The retroviral particles were pseudotyped with VSV-G, displayed the T cell activation element UCHT1-scFvFc-GPI, and were produced by transfecting F1XT cells using the 5 plasmid protocol at the 10 liter intermediate-scale as described in Example 1. Viral supernatants were purified by a combination of depth filtration, TFF, benzonase treatment, diafiltration, and formulation to generate substantially pure viral particles (F1-3-637GU) free of non-human animal proteins.

Whole blood from a healthy volunteer with informed consent was collected into tubes containing heparin. 50 ml were used for each experimental group. No blood cell fractionation or enrichment was performed before the heparinized whole blood was contacted with retroviral particles. 2.5×10⁸ TU of F1-3-637GU (4.87 ml virus with 5.13×10⁷ TU/ml viral particles) was added to 50 ml of heparinzed blood in two groups, such that virus was added at an MOI of 5 based on the assumption of 1.0×10⁶ CD3+ cells/ml of blood. The bags were inverted 5 times to mix the contents, then incubated for 4 hours, at 37° C., 5% CO₂. Following the 4 hour contacting time, 50 ml of control blood that was not contacted with virus (“G2”) and 50 ml F1-3-637GU contacted blood cell sample (“G4”) were separately loaded onto Hematrate leukoreduction filters, washed with saline HSA heparin, and eluted with saline HSA. For enrichment of PBMCs, 50 ml of control blood that was not contacted with virus (“G3”) and 50 ml F1-3-637GU contacted blood cell sample (“G5”) were separately enriched by density gradient centrifugation with Ficoll-Paque™ (General Electric) using a CS-900.2 kit (BioSafe; 1008) on a Sepax 2 S-100 device (Biosafe; 14000) using 2 wash cycles according to the manufacturer's instructions, to obtain 45 ml of isolated PBMCs from each run. The wash and final resuspension solution used in the Sepax 2 process was Normal Saline (Chenixin Pharm)+2% human serum albumin (HSA) (Sichuan Yuanda Shuyang Pharmaceutical). Cells were counted, and 2.5×10⁷ cells from each group were diluted to 2.5×10⁷ cells/ml in normal saline+2% HSA. Following the 4 hour incubation, samples of cells from each of Groups G2, G3, G4, and G5 were also taken for analysis by flow cytometry as discussed in Example 8.

The ability of anti-CD19 self-driving CAR-T cells to treat a model of systemic Human Burkitt's Lymphoma was examined in a mouse model. Female NSG mice were used in this study. Each mouse was inoculated with 3.0×10⁵ Raji-Luciferase cells in 100 μl of PBS via intravenous tail vein injection for tumor development on day −4. Raji cells naturally express CD19. 25 mice were randomly allocated into 5 groups (5 mice/group) for administration of test articles in 200 μl subcutaneously. Mice in each group received the following test articles on Day 0: G1, PBS; G2, 5×10⁶ untransduced TNC; G3, 5.0×10⁶ untransduced PBMCs G4; 5.0×10⁶ TNC transduced with F1-3-637; and G5, 5×10⁶ PBMCs transduced with F1-3-637GU.

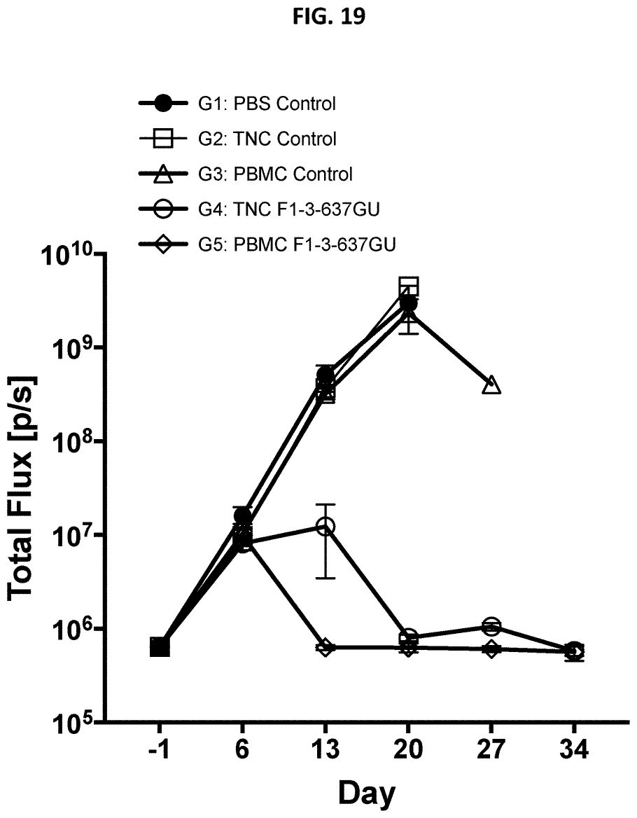

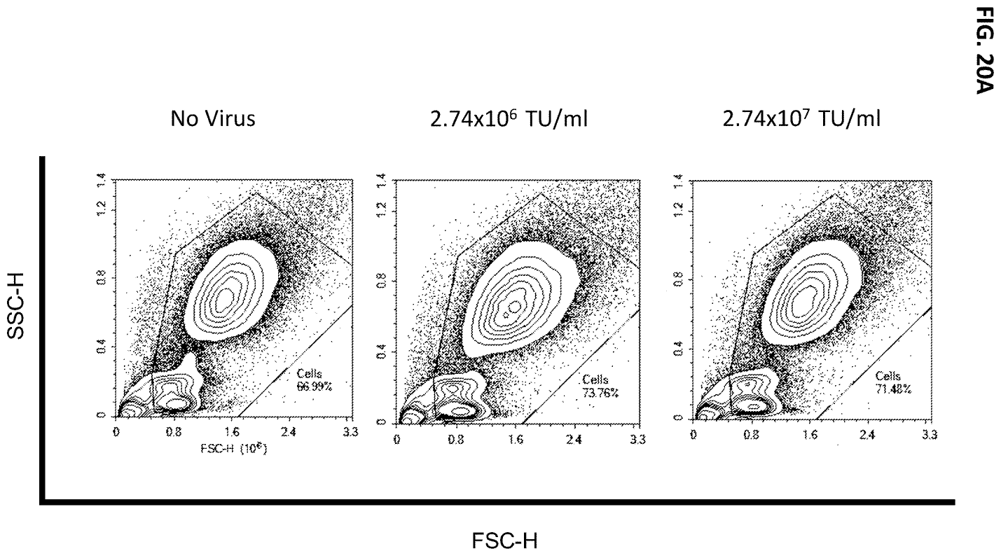

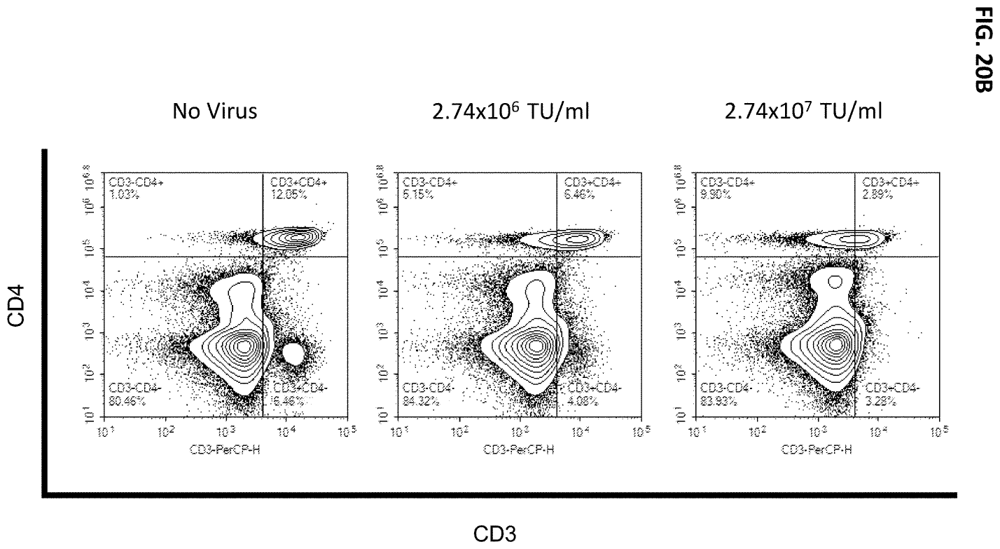

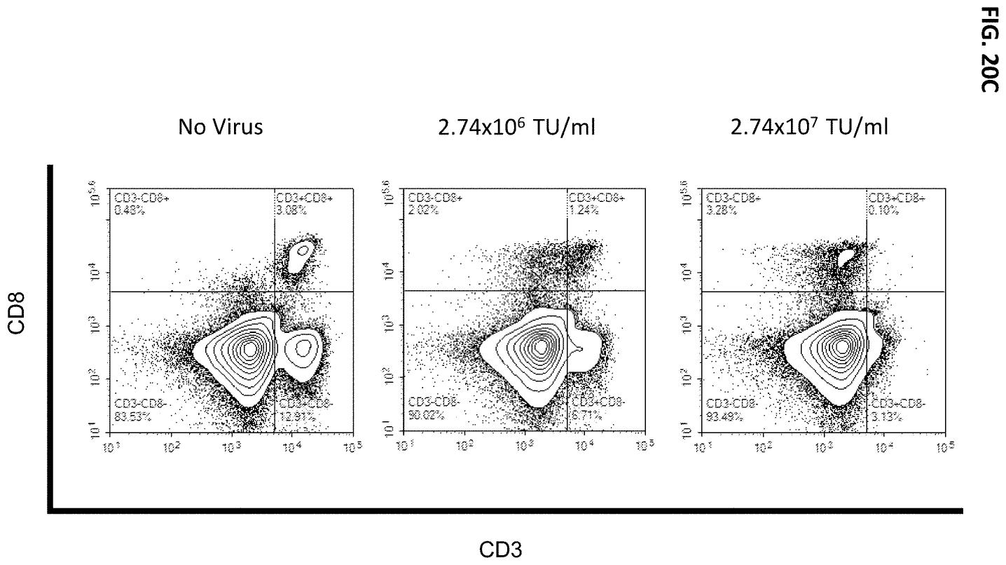

Mice were assessed for tumor growth by bioluminescent imaging (PerkinElmer, IVIS Lumina Series II) and analyzed with LivingImage software. As shown in FIG. 19, both TNC and PBMCs transduced with F1-3-637GU cleared systemic Raji tumors by day 20 post dosing. In contrast, the tumor burden in mice that received TNC or PBMC controls mock transduced with PBS continued to increase during the study as measured by total flux. Tumor burden in G3 showed some tumor regression on day 27. This is believed to be the result of graft versus host disease.

This example demonstrates that lentiviral particles encoding bicistronic genomic vectors and displaying the activation element UCHT1-scFvFc-GPI on their surface, when incubated with whole blood for 4 hours, can transduce PBMCs or TNCs and be effectively administered into subjects to elicit an anti-tumor effect. When delivered subcutaneously, both transduced PBMCs and TNCs, which were self driving CARs expressing a lymphoproliferative element and a CAR directed to CD19, were capable of expanding in vivo and eliminating systemic Raji tumors.

### Example 8. Contacting a Population of Cells Comprising T Cells with Viral Particles Displaying a CD3 T Cell Activation Element on Their Surface Leads to a Reduction in the Percentage of Cells That Express the TCR Complex on Their Surface

In this example, cell populations of differing compositions were contacted for 4 hours with varying concentrations of viral particles displaying a T cell activation element directed to CD3, and surface expression of the TCR complex was analyzed by flow cytometry and quantitated. Downregulation of surface CD3 expression and the appearance of CD3-CD4+ and CD3-CD8+ cell populations were identified in whole blood, PBMCs, and TNCs.

In a first experiment, a dose titration of F1-3-247GU lentiviral particles produced as described in Example 3 were added to whole blood to observe the effects of increasing virus concentration on the loss of surface CD3 expression. Whole blood from healthy volunteers was collected and 100 μl was aliquoted into the wells of a 96 deep-well plate. 120 μl F1-3-247GU viral particles in PBS-4% Lactose was added to the wells at a final concentration of 2.74E+07, 1.37E+07, 6.86E+06, 2.74E+06, 1.37E+06, 2.74E+05, or zero (PBS-4% Lactose control) TU/ml (n=6). The reactions were incubated for 4 hours at 37° C. and 5% CO₂. After the 4 hour contacting, the cells were minimally processed by lysing RBCs; no PBMC or TNC isolation steps were performed. The cells were then stained with anti-CD3-PerCP (SK7) (BD, 347344), anti-CD8-FITC (SK1) (BD, 347313), and anti-CD4-PE (SK3) (BD, 347327) and analyzed by flow cytometry.

Representative FACS profiles with the concentration of virus used for the contacting is shown in FIG. 20. FIG. 20A is a plot of FSC versus SSC showing the gate, “Cells” used to analyze the cells for the expression of CD3, CD4, and CD8. As shown in FIG. 20B, the percentage of CD3+CD4+ cells decreased and the percentage of CD3-CD4+ cells increased with increasing concentration of virus. Similarly, as shown in FIG. 20C, the percentage of CD3+CD8+ cells decreased and the percentage of CD3-CD8+ cells increased with increasing concentration of virus.

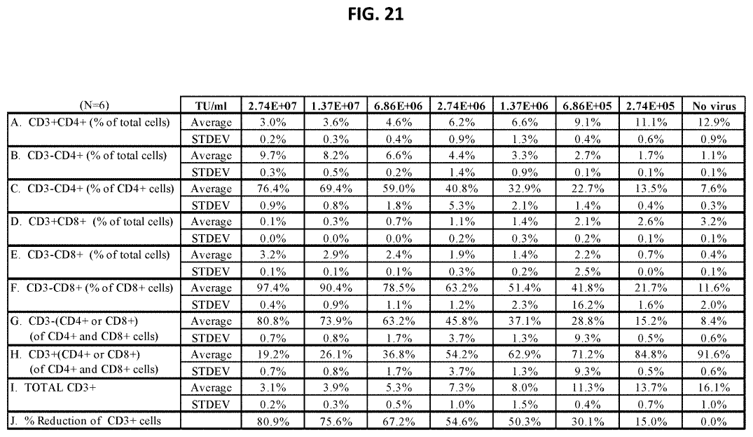

The results of the first experiment are shown in more detail in the table in FIG. 21. The column on the right shows the average percentages and standard deviations (n=6) of each population of cells in the absence of contacting with virus. Following contacting with virus, an increase or decrease in the percentage of a population greater than 3 standard deviations from the mean was deemed to be significant.

Row A shows that CD3+CD4+ cells as a percentage of the total cells ranged from 11.1% to 3.0% as the concentration of virus increased. When compared to no viral contacting, a significant decrease in the percentage of CD3+CD4+ cells was observed such that less than 10% of the total cells were CD3+CD4+following contacting by virus for all but the lowest concentration of virus tested.

Row B shows that CD3-CD4+ cells as a percentage of the total cells ranged from 1.7% to 9.7% as the concentration of virus increased. When compared to no viral contacting, a significant increase in the percentage of CD3-CD4+ cells was observed such that more than 1.5% of the total cells were CD3-CD4+following contacting by virus for all concentrations of virus tested.

Row C shows that CD3-CD4+ cells as a percentage of CD4+ cells ranged from 13.5% to 76.4% as the concentration of virus increased. When compared to no viral contacting, a significant increase in the percentage of CD3-CD4+ cells was observed such that more than 9% of CD4+ cells were CD3− following contacting by virus for all concentrations of virus tested. CD3− cells as a percentage of CD4+ cells was calculated as % CD3-CD4+/[(% CD3-CD4+)+[(% CD3+CD4+)].

Row D shows that CD3+CD8+ cells as a percentage of the total cells ranged from 2.6% to 0.1% as the concentration of virus increased. When compared to no viral contacting, a significant decrease in the percentage of CD3+CD8+ cells was observed such that less than 2.5% of the total cells were CD3+CD8+following contacting by virus for all but the lowest concentration of virus tested.

Row E shows that CD3-CD8+ cells as a percentage of the total cells ranged from 0.7% to 3.2% as the concentration of virus increased. When compared to no viral contacting, a significant increase in the percentage of CD3-CD8+ cells was observed such that more than 0.6% of the total cells were CD3-CD8+following contacting by virus for all concentrations of virus tested.

Row F shows that CD3-CD8+ cells as a percentage of CD8+ cells ranged from 21.7% to 97.4% as the concentration of virus increased. When compared to no viral contacting, a significant increase in the percentage of CD3-CD8+ cells was observed such that more than 18% of CD8+ cells were CD3− following contacting by virus for all concentrations of virus tested. CD3− cells as a percentage of CD8+ cells was calculated as % CD3-CD8+/[(% CD3-CD8+)+[(% CD3+CD8+)].

Row G shows that CD3− and (CD4+ or CD8+) cells as a percentage of the total of CD4+ cells and CD8+ cells ranged from 15.2% to 80.8% as the concentration of virus increased. When compared to no viral contacting, a significant increase in the percentage of CD3− and (CD4+ or CD8+) cells was observed such that more than 10.5% of the total of CD4+ cells and CD8+ cells were CD3− and (CD4+ or CD8+) following contacting by virus for all concentrations of virus tested. CD3− and (CD4+ or CD8+) cells as a percentage of the total of CD4+ cells and CD8+ cells was calculated as [(% CD3-CD4+)+(% CD3-CD8+)]/[(% CD3-CD4+)+(% CD3-CD8+)+(% CD3-CD4+)+(% CD3-CD8+)]

Row H shows that CD3+ and (CD4+ or CD8+) cells as a percentage of the total of CD4+ cells and CD8+ cells ranged from 84.8% to 19.2% as the concentration of virus increased. When compared to no viral contacting, a significant increase in the percentage of CD3− and (CD4+ or CD8+) cells was observed such that more than 10.5% of the total of CD4+ cells and CD8+ cells were CD3− and (CD4+ or CD8+) following contacting by virus for all concentrations of virus tested. CD3− and (CD4+ or CD8+) cells as a percentage of the total of CD4+ cells and CD8+ cells was calculated as [(% CD3-CD4+)+(% CD3-CD8+)]/[(% CD3-CD4+)+(% CD3-CD8+)+(% CD3-CD4+)+(% CD3-CD8+)]

Row I shows that total CD3+ cells as a percentage of the total cells ranged from 13.7% to 3.1% as the concentration of virus increased. When compared to no viral contacting, a significant decrease in the percentage of total CD3+ cells was observed such that less than 13% of the total cells were CD3+ following contacting by virus for all but the lowest concentration of virus tested.

Row J shows the percentage reduction of total CD3+ cells between viral contacting and no viral contacting ranged from 15% to 80.9% as the concentration of virus increased. When compared to no viral contacting, a significant decrease in the percentage reduction of total CD3+ cells was observed such that more than a 19% reduction in the % CD3+ cells following viral contacting as compared to no viral contacting was observed for all but the lowest concentration of virus tested. The percent reduction in CD3+ cells was calculated as [(% Total CD3+ for no virus)−((% Total CD3+ for with virus)]/(% Total CD3+ for no virus).

In a second experiment, the cells were obtained from the experiment in Example 7. In brief, 2.5×10⁸ TU (4.87 ml of virus at 5.13×10⁷ TU/ml) of F1-3-247GU RIPs were added to each of 2 bags containing 50 ml of heparinzed blood (4.6×10⁶ TU/ml final concentration). The reaction mixture was incubated for 4 hours at 37° C. and 5% CO₂ The blood in one bag was processed to enrich for TNCs, while blood in the other bag was processed to enrich for PBMCs as described in Example 7. No further incubation or processing was performed before the cells were prepared for flow cytometry. The cells were stained for human CD3 (OKT3 Brilliant Violet 421, BioLegend 317344), CD4 (OKT4 PE/Cyanine5, BioLegend 317412), and CD8 (RPA-T8 Brilliant Violet 510, BioLegend 301048).

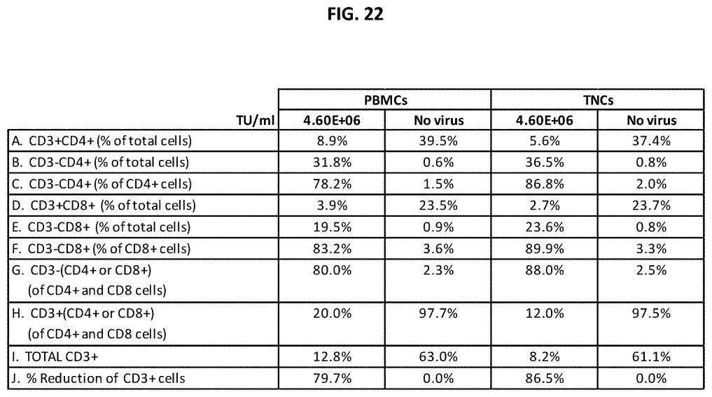

In this second experiment, the cells were analyzed by gating for singlets based on FSC-A and FSC-H, and lymphocytes based on FSC-A and SSC-A, before being analyzed for expression of CD3, CD4, and CD8. The results of the second experiment are shown in more detail in the table in FIG. 22. Again a significant reduction in surface expression of CD3 was observed in the samples. As shown in row C, within the CD4+ population, the percentage of CD3− cells was 78.2% and 86.8%, for the PBMC and TNC preparations, respectively after viral contacting. As shown in row F, within the CD8+ population, the percentage of CD3− cells was 83.2% and 89.9%, for the PBMC and TNC preparations, respectively after viral contacting. As shown in row G, within the collective populations of CD4+ and CD8+ T cell populations, the percentage of CD3− cells was 80.0% and 88.0%, for the PBMC and TNC preparations, respectively after viral contacting. As shown in row H, within the collective populations of CD4+ and CD8+ T cell populations, the percentage of CD3+ cells was 20.0% and 12.0%, for the PBMC and TNC preparations, respectively after viral contacting. Finally, as shown in row J, the percent reduction of CD3+ cells between untreated samples and those contacted by virus was 79.7% and 86.5% for the PBMC and TNC preparations, respectively. When comparing the percent CD3+ or CD3− populations between experiments, looking within the CD4+ and/or CD8+ population gives greater precision than looking at CD3 expression as a percentage of total cells (as in rows A, B, D, and E) which is more sensitive to different blood donors, cell processing methods, and FACs gating.

In a third experiment, 1.17×10⁹ TU (5.9 ml of virus at 1.98×10⁸ TU/ml) of recombinant lentiviral particles encoding VP221 pseudotyped with VSV-G and displaying the T cell activation element, UCHT1-scFvFc-GPI (VP221GU) were added to 20 ml of heparinized blood (4.51×10⁷ TU/ml final concentration). The reaction mixture was incubated for 4 hours at 37° C. and 5% CO₂ Total nucleated cells (TNCs) were subsequently captured from the transduction reaction mixture on a leukoreduction filter, washed, and collected by reverse perfusion of the leukoreduction filter assembly. The cell processing workflow was as shown in FIG. 1D with the exceptions that the optional steps of 170D and 180D were not performed, only portions of the process were performed in a closed system, and a portion of the final cells of step 160D were processed for flow cytometry. The cells were stained for human CD4 (OKT4 PE/Cyanine5, BioLegend 317412), and CD8 (RPA-T8 Brilliant Violet 605, BioLegend 301040) and one of the following antibodies to the TCR complex; CD3 (HIT3a Pacific Blue, BioLegend 300330), CD3 (UCHT1 Brilliant Violet 421, BioLegend 300434), CD3 (SK7 Brilliant Violet 421, BioLegend 344834), CD3 (OKT3 Brilliant Violet 421, BioLegend 317344), or TCRα/β (IP26 Brilliant Violet 785, BioLegend 306742).

Similar to the gate used in first experiment in this example, a “Cells” gate was used to analyze the cells in this third experiment for the expression of CD3, CD4, and CD8. The percent reduction of CD3+ cells between untreated samples and those contacted by virus was 89.2%, 99.8%, 95.5%, and 92.4% when stained with OKT3, UCHT1, SK7, and HIT3a, respectively. That staining with UCHT1, the same antibody that is displayed on the virus surface, shows the greatest reduction in detectable surface CD3 expression suggests that there may be some epitope masking by the virus. Nevertheless, these results indicate that there is a significant reduction of surface expression of CD3 regardless of the CD3 epitope that is interrogated. Furthermore, the percent reduction of TCRα/β was 83.5% when stained with IP26 indicating that surface expression of the entire TCR complex is reduced following contacting with recombinant retroviral particles displaying a T cell activation element that binds CD3. This reduction in surface expression of the TCR complex, or dimming, following contacting with retroviral particles has been observed with every experiment in which a population of cells that includes T cells was contacted with retroviral particles that display an activation element capable of binding to CD3. Furthermore, this dimming occurs when the contacting occurs in whole blood or when the blood is fractionated into PBMCs or TNCs either before or after the contacting. Based on these results, the inventors believe that any activation element capable of crosslinking the TCR would also lead to a reduction in surface expression of the TCR complex. This is consistent with activation of the TCR complex resulting in internalization of the receptor complex.

### Example 9. Biodistribution Analysis of Lymphocytes Injected Subcutaneously Demonstrate Significantly Superior Engraftment and Persistence as Compared to Lymphocytes Injected Intravenously

In this example, the biodistribution of lymphocytes delivered by either subcutaneous injection or intravenous injection, were compared side-by-side by bioluminescent imaging.

Human T cells and NKT cells were effectively genetically modified by a 4 hour incubation of a reaction mixture that included whole blood and substantially pure F1-3-748GU viral particles free of non-human animal proteins at an MOI of 5. The F1-3-748 bicistronic vector encodes a CD19 CAR, a lymphoproliferative element, and luciferase as disclosed in more detail in Example 1. The viral particles in this Example were pseudotyped with VSV-G and displayed a T cell activation element on their surface. Following the incubation, TNCs were captured from the transduction reaction mixture on a leukoreduction filter, washed, and collected in 2% HSA normal saline by reverse perfusion of the leukoreduction filter assembly. The rPOC cell processing workflow was as shown in FIG. 1D with the exceptions that the optional steps of 170D and 180D were not performed, and only portions of the process were performed in a closed system 5 million of the modified TNCs suspended in 200 μl in 2% HSA normal saline were then injected into 6-8 week old female B-NDG mice with established solid Raji tumors averaging 150 mm³. Five mice were injected intravenously and five mice were injected subcutaneously in the flank opposite the tumor. The in vivo biodistribution of TNCs transduced with F1-3-748 expressed luciferase and were followed by bioluminescent imaging (PerkinElmer, IVIS Lumina Series II) and analyzed with LivingImage software.

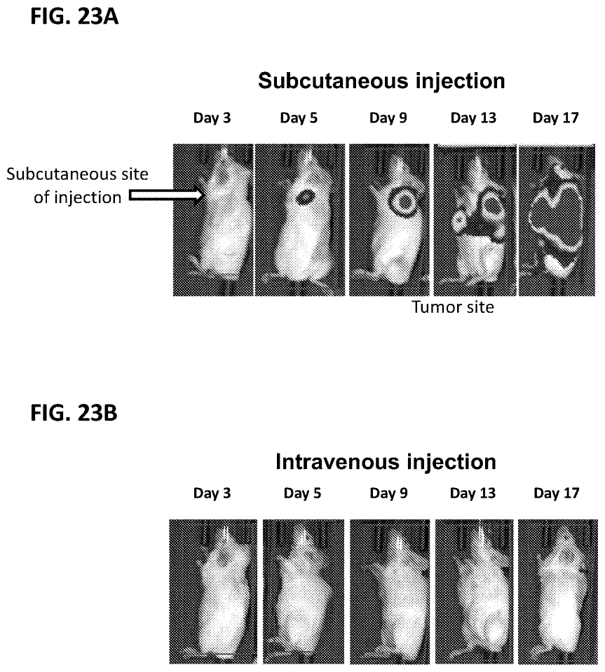

IVIS images of representative mice from each group are shown in FIG. 23. As shown in FIG. 23A, when the TNCs were injected subcutaneously, no genetically modified lymphocytes expressing the luciferase transgene were detected at Day 3. By Day 5, transduced lymphocytes were seen at the site of injection. The Day 9 image shows an increase of transduced lymphocytes at the site of injection, but transduced lymphocytes were detectable elsewhere in the mouse, indicating that some of the cells expressing the transgene migrated out away from the injection site. The amount of cells expressing the luciferase transgene appeared to remain high at or near the subcutaneous site of injection, showing transduced cell expansion and persistence in this area, and the area of these cells appeared to increase and emanate from that injection site, forming what appeared to be a cell concentration gradient centered at or near the site of injection. By Day 13, transduced lymphocytes were present in the expanded area at the site of injection as well as in the tumor, and by Day 17 transduced lymphocyte could be seen throughout the body, although the density of cells expressing the transgene still appeared higher in a larger area around the tumor and site of administration than more distal areas of the mice. These data suggest that when lymphocytes are modified by the rPOC process, most if not all of the reverse transcription, DNA integration, and transgene expression occurs at the site of subcutaneous injection. These lymphocytes proliferated at the site of injection for approximately 5 to 10 days and subsequently migrated into the circulation and trafficked to the tumor. Once in the tumor, these lymphocytes which also express a CD19 CAR, apparently engaged Raji targets and continued to proliferate robustly. Surprisingly, and in stark contrast, when the TNCs were injected intravenously, genetically modified lymphocytes were undetectable by IVIS in the mice, as shown in FIG. 23B.

### Example 10. When Administered Subcutaneously, Lymphocytes Modified by rPOC Cell Processing Methods to Express a CAR Form Tertiary Lymphoid Structures, Engraft, and Kill Target Cells Dispersed Throughout the Body

In this example, PBMCs were effectively genetically modified by a 4 hour incubation with RIPs encoding a CAR and a lymphoproliferative element. The modified PBMCs were then injected subcutaneously into mice engineered to lack murine lymphocytes, but were reconstituted intravenously with human PBMCs such that the mice were lymphoreplete. This model was used in a first experiment to assess the ability of genetically modified PBMCs depleted of CAR target cells and introduced subcutaneously, to engraft in a lymphoreplete host. This model was also used in a second experiment to assess the ability of the genetically modified PBMCs (that had not been depleted of CAR target cells) introduced subcutaneously, to form tertiary lymphoid structures and to kill CAR target cells in the blood.

Recombinant F1-3-247GU lentiviral particles were produced as described in Example 3. F1-3-247 encodes a CD19 CAR comprised of an anti-CD19scFv,a CD8 stalk and transmembrane region, and an intracellular domain from CD3z followed by T2A and the lymphoproliferative element comprising the parts E006-T016-S186-S050, which encode an extracellular domain containing a variant of c-Jun including a leucine zipper motif and an eTAG, the transmembrane domain of CSF2RA, the intracellular domain of MPL, and the intracellular domain of CD40, with each part of the lymphoproliferative element connected by a GGS linker.

Fresh PBMCs depleted of CD19+ cells used in the first experiment and Fresh PBMCs (not depleted of CD19+ cells) used in the second experiment, were obtained from StemExpress. In each case, F1-3-247GU viral particles were added to the PBMCs in a conical tube at an MOI of 2.5 and incubated for 4 hours at 37° C. and 5% CO₂. After the 4 hour contacting, the cells were pelleted for 10 minutes at 418 g and washed 3 times with PBS+2% HSA. The cells were resuspended in PBS+2% HSA at a concentration of 3×10⁷ cells/mL. No exogenous cytokines were added to the samples at any time.

10 and 7 week old female immunodeficient NSG-MHC1/2-DKO (NSG-(K^{b}D^{b})^{null}(IA)^{null}, Jackson Laboratories) mice were used in the first and second experiments in this example, respectively. To test the activity of PBMCs modified with F1-3-247GU in a lymphoreplete host, these mice were reconstituted with a human immune system by injecting 200 μl of human PBMCs at 5.0×10⁷ cells/mL in PBS-HSA (10 million PBMCs) IV in the tail vein. Within each experiment, the PBMCs administered IV and SC were donor matched.

For the first experiment, 6 million PBMCs in 200 μl PBS-HSA that were either transduced with F1-3-247GU or mock transduced with PBS were injected subcutaneously. 5 mice were included in each group. Blood was taken from the mice on Day 21 for analysis of serum cytokines and on Day 27 for analysis by flow cytometry.

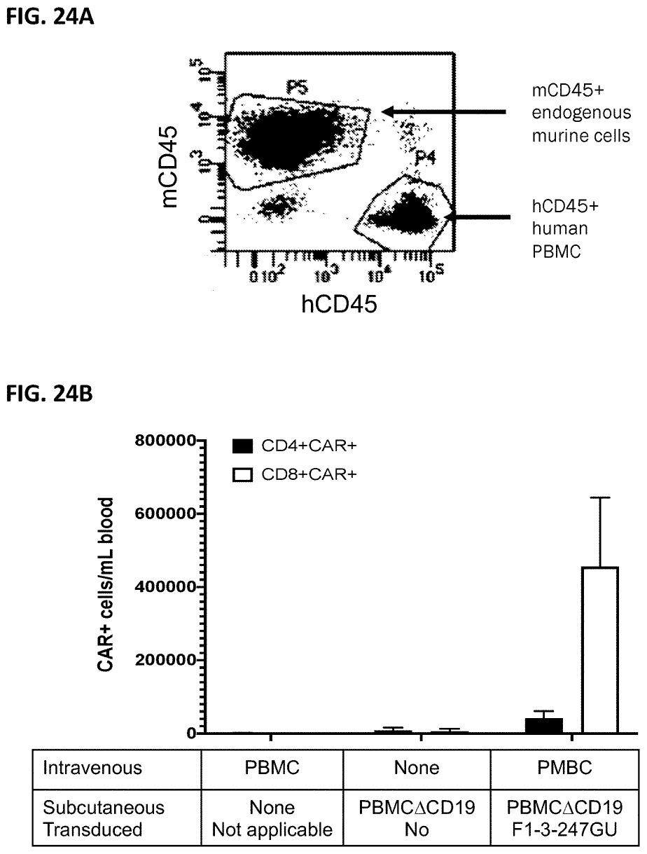

The ability of the human PBMCs injected IV to engraft and reconstitute the immune system of NSG-MHC1/2-DKO mice was assessed by staining for human CD45. The plot in FIG. 24A shows CD45 expression in blood from a representative mouse 27 days after receiving human PBMCs IV and no PBMCs subcutaneously. In addition to the endogenous murine cells expressing CD45, a significant population of human CD45+ cells were detected. These results demonstrate that the immune compartment of the NSG-MHC1/2-DKO mice was reconstituted with human cells. The graph in FIG. 24B shows that significant numbers of T cells expressing the CD19 CAR could be detected in lymphoreplete mice 27 days after dosing subcutaneously with PBMCs depleted of CD19 and genetically modified with F1-3-247GU. While both CD8+ and CD4+ T cells were present, there were approximately 11-fold more CD8+ cells.

Skin and subcutaneous tissue was stained with hematoxylin and eosin (H&E) and antibodies to CD4, CD8, and CD68, to study the distribution of modified PBMCs administered subcutaneously. FIG. 26A shows an H&E stained slide from a sample on Day 1 after dosing. The arrows in FIG. 26A point to small lymphocytes that are scattered throughout the subcutaneous region consistent with the cells having been delivered and retained subcutaneously. FIG. 26B shows that the epidermis of skin is intact with no evidence of ulceration or dermal acute inflammation on Day 7 after the subcutaneous administration, as may occur following an intradermal administration. The arrows in FIG. 26B point to tertiary lymphoid structures (TLSs) with defined borders and a structure that resembles a lymph node.

Immunohistochemistry confirmed that these TLSs included CD4+ lymphocytes, CD8+ lymphocytes, and CD68+ antigen presenting cells. These structures can aid in the maturation and education of T and NK cells outside of secondary lymphoid organs. TLSs were not observed on Day 4 and therefore likely formed between days 4 and 7. FIG. 26C shows that these TLSs continued to grow thru Day 14. When viewed under higher magnification, multinucleated giant cells, activated mononuclear cells, and actively dividing lymphocytes were identified. FIG. 26D shows that on Day 21, residual areas of lymphocytes are present in the subcutaneous region, but TLSs are no longer present. These results are consistent with the biodistrubtion seen in FIG. 23A of Example 9 which shows modified cells dispersing from the site of injection between days 9 and 17, as well as large numbers of modified cells present in the blood between days 14 and 35 in FIG. 7 of Example 3. Importantly, these data also support that TLSs comprising lymphocytes modified with a constitutively active lymphoproliferative element can resolve.

In the second experiment, 3 different doses of PBMCs transduced with F1-3-247GU to express the CD19 CAR and administered subcutaneously were examined for their ability to engraft and kill the PBMCs introduced intravenously. 1 million, 100,000, or 10,000 modified PBMCs were resuspended in 100 μl PBS-HSA and injected subcutaneously. As controls, one group of mice received mock transduced PBMCs and another group received PBS subcutaneously. All mice received 10 million PBMCs intravenously. 5 mice were included in each group. Blood was taken from the mice on Day 21 for quantitation of human CD3-CD19+ cells by flow cytometry. An additional 5 mice were treated as described above in which 1 million modified PBMCs were dosed subcutaneously, and tissue samples were taken at days 1, 4, 7, 14, and 21 for histology.

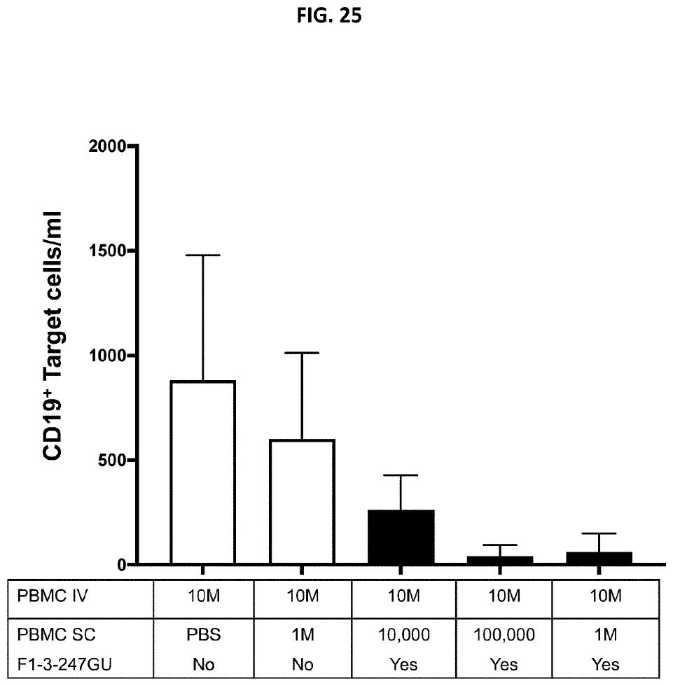

The number of human CD3-CD19+ cells per ml of blood for each group of mice on Day 21 is shown in FIG. 25. On average, mice that received PBS alone subcutaneously had approximately 880 CD19+ cells/ml of blood. On average, mice that received 1 million mock transduced PBMCs subcutaneously had approximately 600 CD19+ cells/ml of blood. On average, mice that received 1 million PBMCs transduced with F1-3-247GU had approximately 60 CD19+ cells/ml of blood. Thus, transduction of the PBMCs with a gene construct encoding a CD19 CAR and administration of 1 million cells subcutaneously resulted in a 10-fold reduction in target cells. Similar results were seen when the mice were dosed with only 100,000 PBMCs. Furthermore, a 2.3-fold reduction in target cells was observed when only 10,000 PMCs were dosed subcutaneously.

This example shows that lymphocytes genetically modified using an rPOC cell process and injected subcutaneously expand, engraft, and kill target cells in a lymphoreplete host. In this example, the lymphocytes were engineered to express a CAR and a lymphoproliferative element. In the first experiment, CD19, the antigen recognized by the CAR, was depleted from the PBMCs before they were transduced. This demonstrates that it is not necessary that the antigen recognized by the CAR be present in the transduction reaction or the subcutaneous milieu in order for the genetically modified cells to expand and engraft. As demonstrated in the second experiment, these cells not only engraft and expand, but they are functionally active and kill target cells even when low doses of 10,000 total PBMCs are dosed subcutaneously in mice.

### Example 11. CARS Directed To HER2 Manufactured by Exposure of Whole Blood to Lentiviral Vectors for 4 Hours Followed by a TNC Enrichment Procedure, When Administered Subcutaneously, Eliminate Solid Tumors in a Human Gastric Cancer Xenograft Model in Mice

In this example, unstimulated human T and NKT cells freshly drawn from peripheral blood were genetically modified by an rPOC cell processing method from heparanized whole blood using replication incompetent recombinant (RIR) retroviral particles encoding a bicistronic genomic vector to generate self-driving CAR cells expressing a second generation CAR directed to HER2 and a lymphoproliferative element. The cell processing workflow was performed as shown in FIG. 1D with the exceptions that the optional steps of 170D and 180D were not performed, and not all steps were performed in a fully closed system. Modified TNC or controls were injected subcutaneously into NSG mice bearing established subcutaneous solid N87 tumors in the opposite flank. Mice were assessed for tumor burden and survival.

Recombinant lentiviral particles used in this example comprised the F1-6-744 bicistronic lentiviral genomic vector. F1-6-744 is described in Example 1. The retroviral particles were pseudotyped with VSV-G, displayed the T cell activation element UCHT1-scFvFc-GPI, and were produced by transfecting F1XT cells using the 5 plasmid protocol at the 10 liter intermediate-scale as described in Example 1. Viral supernatants were purified by a combination of depth filtration, TFF, benzonase treatment, diafiltration, and formulation to generate substantially pure viral particles (F1-6-744GU) free of non-human animal proteins.

Whole blood from a healthy volunteer with informed consent was collected. No blood cell fractionation or enrichment was performed before 63.6 ml of the heparinized whole blood was contacted with 7 ml F1-6-744GU retroviral particles at 8.05E+08 TU (1.14E+07 TU/ml final). The bags were inverted 5 times to mix the contents, then incubated for 4 hours, at 37° C., 5% CO₂. Total nucleated cells (TNCs) were subsequently captured from the transduction reaction mixture on a Hematrate™ leukoreduction filter, washed, and collected by reverse perfusion of the leukoreduction filter assembly. The cell processing workflow was as shown in FIG. 1D with the exceptions that the optional steps of 170D and 180D were not performed, and only portions of the process were performed in a closed system.

NCI-N87 cells which express endogenous human HER2, were utilized to generate a human gastric cancer xenograft model in mice. Subcutaneous (sc) tumor xenografts were established in the hind flank of 7-8 week old female NOD-Prkdc^{scid}Il2rg^{tm1}/Bcgen (B-NDG) mice (Beijing Biocytogen Co. Ltd.). Briefly, cultured N87 cells were washed in DPBS (Thermo Fisher), counted, resuspended in cold DPBS and mixed with an appropriate volume of Matrigel ECM (Corning; final concentration 5 mg/mL) at a concentration of 1.0×10⁷ cells/100 μl Matrigel on ice Animals were prepared for injection using standard approved anesthesia with hair removal (Nair) prior to injection. 100 μl of cell suspension in ECM was injected subcutaneously.

Mice were dosed subcutaneously with 200 μl of test articles in the opposite flank to the tumors, when the tumors averaged 146 mm³. One group of mice received 1 million modified TNC while another group of mice received 5 million modified TNC. Control groups received 5 million unmodified TNCs or PBS alone. Five mice were in each group. Tumors were measured using calipers 2 times a week and tumor volume was calculated using the following equation: (longest diameter*shortest diameter²)/2.

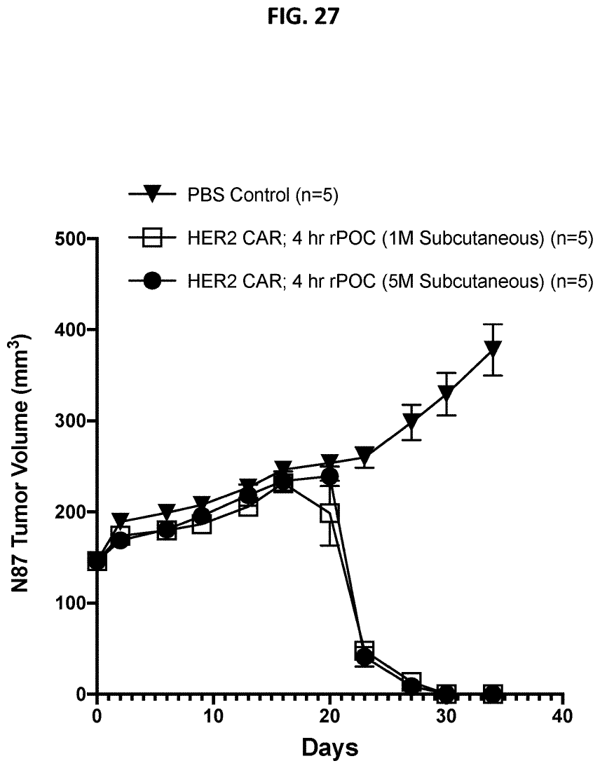

The ability of the test articles to regress established N87 tumors in vivo was examined over time. As shown in FIG. 27, TNCs transduced with F1-6-744 and delivered subcutaneously led to a rapid and dramatic reduction in tumor burden beginning 20 days post dosing. Tumors were undetectable by caliper measurements by 30 days post dosing. Similar tumor regression was observed when either 1 million or 5 million transduced cells were dosed, suggesting that dosing even fewer cells would also result in tumor regression. In contrast, mice dosed with untransduced TNCs exhibited partial tumor regression at later time points (not shown) due to what is believed to have been a graft versus tumor allogeneic response independent of the CAR. Finally, tumors continued to grow in control mice dosed with PBS alone. All of the mice survived to Day 34 at which time the experiment was concluded.

This example demonstrates that lentiviral particles encoding bicistronic genomic vectors and displaying the activation element UCHT1-scFvFc-GPI on their surface, when incubated with whole blood for 4 hours, can transduce lymphocytes. These lymphocytes can be enriched using a leukoreduction filter assembly in a process as shown in FIG. 1D. When delivered subcutaneously, a single dose of these transduced TNCs, which were self-driving CARs expressing a lymphoproliferative element and a CAR directed to HER2, were capable of expanding in vivo in the absence of antigen in the subcutaneous environment, and eliminating solid N87 tumors.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

TABLE 1
Parts, names, and amino acid sequences for domains of lymphoproliferative parts P1-P2, P1, P2,
P3, and P4.

| Part | Name | Amino Acid Sequence |
|---|---|---|
| M001 | eTAG IL7RA Ins PPCL | MLLLVTSLLLCELPHPAFLLIPRKVCNGIGIGEFKDSLSINATNIKHFKNCTSISGDLHILPVAFRGDSFTHTPPLDP |
| | (interleukin 7 receptor) | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGPEINNSSGEMDPILLPPCLTISILSFFSVALLVILACVL (SEQ |
| | | ID NO: 84) |
| M002 | eTAG IL7RA Ins PPCL | MLLLVTSLLLCELPHPAFLLIPRKVCNGIGIGEFKDSLSINATNIKHFKNCTSISGDLHILPVAFRGDSFTHTPPLDP |
| | (interleukin 7 receptor) | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQPEINNSSGEMDPILLPPCLTISILSFFSVALLVILACVL |
| | | (SEQ ID NO: 85) |
| M007 | MycTag LMP1 | MEQKLISEEDLEHDLERGPPGPRRPPRGPPLSSSLGLALLLLLLALLFWLYIVMSDWTGGALLVLYSFALMLIIIIL |
| | NC_007605_l | IIFIFRRDLLCPLGALCILLLMITLLLIALWNLHGQALFLGIVLFIFGCLLVLGIWIYLLEMLWRLGATIWQLLAFFLA |
| | | FFLDLILLIIALYLQQNWWTLLVDLLWLLLFLAILIWM (SEQ ID NO: 86) |
| M008 | Myc LMP1 | MEQKLISEEDLSSSLGLALLLLLLALLFWLYIVMSDWTGGALLVLYSFALMLIIIILIIFIFRRDLLCPLGALCILLLMI |
| | NC_007605_1 | TLLLIALWNLHGQALFLGIVLFIFGCLLVLGIWIYLLEMLWRLGATIWQLLAFFLAFFLDLILLIIALYLQQNWWT |
| | | LLVDLLWLLLFLAILIWM (SEQ ID NO: 87) |
| M009 | LMP1 NC_007605_1 | MEHDLERGPPGPRRPPRGPPLSSSLGLALLLLLLALLFWLYIVMSDWTGGALLVLYSFALMLIIIILIIFIFRRDLLC |
| | | PLGALCILLLMITLLLIALWNLHGQALFLGIVLFIFGCLLVLGIWIYLLEMLWRLGATIWQLLAFFLAFFLDLILLIIA |
| | | LYLQQNWWTLLVDLLWLLLFLAILIWM (SEQ ID NO: 88) |
| M010 | LMP1 NC_007605_1 | MSLGLALLLLLLALLFWLYIVMSDWTGGALLVLYSFALMLIIIILIIFIFRRDLLCPLGALCILLLMITLLLIALWNLHG |
| | | QALFLGIVLFIFGCLLVLGIWIYLLEMLWRLGATIWQLLAFFLAFFLDLILLIIALYLQQNWWTLLVDLLWLLLFLA |
| | | ILIWM (SEQ ID NO: 89) |
| M012 | eTAG CRLF2 transcript | MLLLVTSLLLCELPHPAFLLIPRKVCNGIGIGEFKDSLSINATNIKHFKNCTSISGDLHILPVAFRGDSFTHTPPLDP |
| | variant 1 NM_022148_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGAETPTPPKPKLSKCILISSLAILLMVSLLLLSLW (SEQ ID |
| | | NO: 90) |
| M013 | eTAG CRLF2 transcript | MLLLVTSLLLCELPHPAFLLIPRKVCNGIGIGEFKDSLSINATNIKHFKNCTSISGDLHILPVAFRGDSFTHTPPLDP |
| | variant 1 NM_022148_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQAETPTPPKPKLSKCILISSLAILLMVSLLLLSLW (SEQ |
| | | ID NO: 91) |
| M018 | eTAG CSF2RB | MLLLVTSLLLCELPHPAFLLIPRKVCNGIGIGEFKDSLSINATNIKHFKNCTSISGDLHILPVAFRGDSFTHTPPLDP |
| | NM_000395_2 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGTESVLPMWVLALIEIFLTIAVLLAL (SEQ ID NO: 92) |
| M019 | eTAG CSF2RB | MLLLVTSLLLCELPHPAFLLIPRKVCNGIGIGEFKDSLSINATNIKHFKNCTSISGDLHILPVAFRGDSFTHTPPLDP |
| | NM_000395_2 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQTESVLPMWVLALIEIFLTIAVLLAL (SEQ ID NO: 93) |
| M024 | eTAG CSF3R transcript | MLLLVTSLLLCELPHPAFLLIPRKVCNGIGIGEFKDSLSINATNIKHFKNCTSISGDLHILPVAFRGDSFTHTPPLDP |
| | variant 1 NM_000760_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGTPEGSELHIILGLFGLLLLLNCLCGTAWLCC (SEQ ID NO: 94) |
| M025 | eTAG CSF3R transcript | MLLLVTSLLLCELPHPAFLLIPRKVCNGIGIGEFKDSLSINATNIKHFKNCTSISGDLHILPVAFRGDSFTHTPPLDP |
| | variant 1 NM_000760_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQTPEGSELHIILGLFGLLLLLNCLCGTAWLCC (SEQ ID |
| | | NO: 95) |
| M030 | eTAG EPOR transcript | MLLLVTSLLLCELPHPAFLLIPRKVCNGIGIGEFKDSLSINATNIKHFKNCTSISGDLHILPVAFRGDSFTHTPPLDP |
| | variant 1 NM_000121_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGTPSDLDPCCLTLSLILVVILVLLTVLALLS (SEQ ID NO: 96) |
| M031 | eTAG EPOR transcript | MLLLVTSLLLCELPHPAFLLIPRKVCNGIGIGEFKDSLSINATNIKHFKNCTSISGDLHILPVAFRGDSFTHTPPLDP |
| | variant 1 NM_000121_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQTPSDLDPCCLTLSLILVVILVLLTVLALLS (SEQ ID |
| | | NO: 97) |
| M036 | eTAG GHR transcript | MLLLVTSLLLCELPHPAFLLIPRKVCNGIGIGEFKDSLSINATNIKHFKNCTSISGDLHILPVAFRGDSFTHTPPLDP |
| | variant 1 NM_000163_4 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGTLPQMSQFTCCEDFYFPWLLCIIFGIFGLTVMLFVFLFS |
| | | (SEQ ID NO: 98) |
| M037 | eTAG GHR transcript | MLLLVTSLLLCELPHPAFLLIPRKVCNGIGIGEFKDSLSINATNIKHFKNCTSISGDLHILPVAFRGDSFTHTPPLDP |
| | variant 1 NM_000163_4 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQTLPQMSQFTCCEDFYFPWLLCIIFGIFGLTVMLFVF |
| | | LFS (SEQ ID NO: 99) |
| M042 | eTAG truncated after Fn | MLLLVTSLLLCELPHPAFLLIPRKVCNGIGIGEFKDSLSINATNIKHFKNCTSISGDLHILPVAFRGDSFTHTPPLDP |
| | F523C IL27RA | QELDILKTVKEITGFLLIQAWPENRTDLHAFENLEIIRGRTKQHGQFSLAVVSLNITSLGLRSLKEISDGDVIISGN |
| | NM_004843_3 | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGHLPDNTLRWKVLPGILCLWGLFLLGCGLSLA (SEQ ID |
| | | NO: 100) |
| M043 | eTAG truncated after Fn | MLLLVTSLLLCELPHPAFLLIPRKVCNGIGIGEFKDSLSINATNIKHFKNCTSISGDLHILPVAFRGDSFTHTPPLDP |
| | F523C IL27RA | QELDILKTVKEITGFLLIQAWPENRTDLHAFENLEIIRGRTKQHGQFSLAVVSLNITSLGLRSLKEISDGDVIISGN |
| | NM_004843_3 | KNLCYANTINWKKLFGTSGQKTKIISNRGENSCKATGQHLPDNTLRWKVLPGILCLWGLFLLGCGLSLA (SEQ |
| | | ID NO: 101) |
| M048 | eTAG truncated after Fn | MLLLVTSLLLCELPHPAFLLIPRKVCNGIGIGEFKDSLSINATNIKHFKNCTSISGDLHILPVAFRGDSFTHTPPLDP |
| | S505N MPL | QELDILKTVKEITGFLLIQAWPENRTDLHAFENLEIIRGRTKQHGQFSLAVVSLNITSLGLRSLKEISDGDVIISGN |
| | NM_005373_2 | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGETATETAWISLVTALHLVLGLNAVLGLLLL (SEQ ID |
| | | NO: 102) |
| M049 | eTAG truncated after Fn | MLLLVTSLLLCELPHPAFLLIPRKVCNGIGIGEFKDSLSINATNIKHFKNCTSISGDLHILPVAFRGDSFTHTPPLDP |
| | S505N MPL | QELDILKTVKEITGFLLIQAWPENRTDLHAFENLEIIRGRTKQHGQFSLAVVSLNITSLGLRSLKEISDGDVIISGN |
| | NM_005373_2 | KNLCYANTINWKKLFGTSGQKTKIISNRGENSCKATGQETATETAWISLVTALHLVLGLNAVLGLLLL (SEQ ID |
| | | NO: 103) |
| E006 | eTag 0A JUN | MLLLVTSLLLCELPHPAFLLIPRKVCNGIGIGEFKDSLSINATNIKHFKNCTSISGDLHILPVAFRGDSFTHTPPLDP |
| | NM_002228_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGLERIARLEEKVKTLKAQNSELASTANMLREQVAQLKQKV |
| | | (SEQ ID NO: 104) |
| E007 | eTag 1A JUN | MLLLVTSLLLCELPHPAFLLIPRKVCNGIGIGEFKDSLSINATNIKHFKNCTSISGDLHILPVAFRGDSFTHTPPLDP |
| | NM_002228_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGLERIARLEEKVKTLKAQNSELASTANMLREQVAQLKQKVA |
| | | (SEQ ID NO: 105) |
| E008 | eTag 2A JUN | MLLLVTSLLLCELPHPAFLLIPRKVCNGIGIGEFKDSLSINATNIKHFKNCTSISGDLHILPVAFRGDSFTHTPPLDP |
| | NM_002228_3 | QELDILKTVKEITGFLLIQAWPENRTDLHAFENLEIIRGRTKQHGQFSLAVVSLNITSLGLRSLKEISDGDVIISGN |
| | | KNLCYANTINWKKFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGLERIARLEEKVKTLKAQNSELASTANMLREQVAQLKQKVA |
| | | A (SEQ ID NO: 106) |
| E009 | eTag 3A JUN | MLLLVTSLLLCELPHPAFLLIPRKVCNGIGIGEFKDSLSINATNIKHFKNCTSISGDLHILPVAFRGDSFTHTPPLDP |
| | NM_002228_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGLERIARLEEKVKTLKAQNSELASTANMLREQVAQLKQKVA |
| | | AA (SEQ ID NO: 107) |
| E010 | eTag 4A JUN | MLLLVTSLLLCELPHPAFLLIPRKVCNGIGIGEFKDSLSINATNIKHFKNCTSISGDLHILPVAFRGDSFTHTPPLDP |
| | NM_002228_3 | QELDILKTVKEITGFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGLERIARLEEKVKTLKAQNSELASTANMLREQVAQLKQKVA |
| | | AAA (SEQ ID NO: 108) |
| E011 | MycTag 0A JUN | MTILGTTFGMVFSLLQVVSGEQKLISEEDLLERIARLEEKVKTLKAQNSELASTANMLREQVAQLKQKV (SEQ |
| | NM_002228 3 | ID NO: 109) |
| E012 | MycTag 1A JUN | MTILGTTFGMVFSLLQVVSGEQKLISEEDLLERIARLEEKVKTLKAQNSELASTANMLREQVAQLKQKVA (SEQ |
| | NM_002228_3 | ID NO: 110) |
| E013 | MycTag 2A JUN | MTILGTTFGMVFSLLQVVSGEQKLISEEDLLERIARLEEKVKTLKAQNSELASTANMLREQVAQLKQKVAA |
| | NM_002228 3 | (SEQ ID NO: 111) |
| E014 | MycTag 3A JUN | MTILGTTFGMVFSLLQVVSGEQKLISEEDLLERIARLEEKVKTLKAQNSELASTANMLREQVAQLKQKVAAA |
| | NM_002228_3 | (SEQ ID NO: 112) |
| E015 | MycTag 4A JUN | MTILGTTFGMVFSLLQVVSGEQKLISEEDLLERIARLEEKVKTLKAQNSELASTANMLREQVAQLKQKVAAAA |
| | NM_002228 3 | (SEQ ID NO: 113) |
| E016 | 0A JUN NM_002228_3 | LERIARLEEKVKTLKAQNSELASTANMLREQVAQLKQKV |
| T001 | CD2 transcript variant 1 | LIIGICGGGSLLMVFVALLVFYI (SEQ ID NO: 114) |
| | NM_001328609 1 | |
| T002 | CD3D transcript variant | GIIVTDVIATLLLALGVFCFA (SEQ ID NO: 115) |
| | 1 NM_000732_4 | |
| T003 | CD3E NM_000733_3 | VMSVATIVIVDICITGGLLLLVYYWS (SEQ ID NO: 116) |
| T004 | CD3G NM_000073_2 | GFLFAEIVSIFVLAVGVYFIA (SEQ ID NO: 117) |
| T005 | CD3Z CD247 transcript | LCYLLDGILFIYGVILTALFL (SEQ ID NO: 118) |
| | variant 1 NM_198053_2 | |
| T006 | CD4 transcript variant 1 | MALIVLGGVAGLLLFIGFIGLGIFF (SEQ ID NO: 119) |
| | and 2 NM_000616 4 | |
| T007 | CD8A transcript variant | IYIWAPLAGTCGVLLLSLVIT (SEQ ID NO: 120) |
| | 1 NM_001768_6 | |
| T008 | CD8B transcript variant | LGLLVAGVLVLLVSLGVAIHLCC (SEQ ID NO: 121) |
| | 2 NM_172213 3 | |
| T009 | CD27 NM_001242_4 | ILVIFSGMFLVFTLAGALFLH (SEQ ID NO: 122) |
| T010 | CD28 transcript variant | FWVLVVVGGVLACYSLLVTVAFIIFWV (SEQ ID NO: 123) |
| | 1 NM_006139_3 | |
| T011 | CD40 transcript variant | ALVVIPIIFGILFAILLVLVFI (SEQ ID NO: 124) |
| | 1 and 6 NM_001250_5 | |
| T012 | CD79A transcript | IITAEGIILLFCAVVPGTLLLF (SEQ ID NO: 125) |
| | variant 1 NM_001783_3 | |
| T013 | CD79B transcript | G11MIQTLLIILFIIVPIFLLL (SEQ ID NO: 126) |
| | variant 3 | |
| | NM_001039933_2 | |
| T014 | CRLF2 transcript variant | FILISSLAILLMVSLLLLSLW (SEQ ID NO: 127) |
| | 1 NM_022148_3 | |
| T015 | CRLF2 transcript variant | CILISSLAILLMVSLLLLSLW (SEQ ID NO: 128) |
| | 1 NM_022148_3 | |
| T016 | CSF2RA transcript | NLGSVYIYVLLIVGTLVCGIVLGFLF (SEQ ID NO: 129) |
| | variant 7 and 8 | |
| | NM_001161529_1 | |
| T017 | CSF2RB NM_000395_2 | MWVLALIVIFLTIAVLLAL (SEQ ID NO: 130) |
| T018 | CSF2RB NM_000395_2 | MWVLALIEIFLTIAVLLAL (SEQ ID NO: 131) |
| T019 | CSF3R transcript variant | IILGLFGLLLLLTCLCGTAWLCC (SEQ ID NO: 132) |
| | 1 NM_000760_3 | |
| T020 | CSF3R transcript variant | IILGLFGLLLLLNCLCGTAWLCC (SEQ ID NO: 133) |
| | 1 NM_000760_3 | |
| T021 | EPOR transcript variant | LILTLSLILVVILVLLTVLALLS (SEQ ID NO: 134) |
| | 1 NM_000121_3 | |
| T022 | EPOR transcript variant | CCLTLSLILVVILVLLTVLALLS (SEQ ID NO: 135) |
| | 1 NM_000121_3 | |
| T023 | FCERIG NM_004106_1 | LCYILDAILFLYGIVLTLLYC (SEQ ID NO: 136) |
| T024 | FCGR2C NM_201563_5 | IIVAVVTGIAVAAIVAAVVALIY (SEQ ID NO: 137) |
| T025 | FCGRA2 transcript | IIVAVVIATAVAAIVAAVVALIY (SEQ ID NO: 138) |
| | variant 1 | |
| | NM_001136219_1 | |
| T026 | GHR transcript variant 1 | FPWLLIIIFGIFGLTVMLFVFLFS (SEQ ID NO: 139) |
| | NM_000163_4 | |
| T027 | GHR transcript variant 1 | FPWLLCIIFGIFGLTVMLFVFLFS (SEQ ID NO: 140) |
| | NM_000163_4 | |
| T028 | ICOS NM_012092.3 | FWLPIGCAAFVVVCILGCILI (SEQ ID NO: 141) |
| T029 | IFNAR1 NM_000629_2 | IWLIVGICIALFALPFVIYAA (SEQ ID NO: 142) |
| T030 | IFNAR2 transcript | IGGIITVFLIALVLTSTIVTL (SEQ ID NO: 143) |
| | variant 1 NM_207585_2 | |
| T031 | IFNGR1 NM_000416_2 | SLWIPVVAALLLFLVLSLVFI (SEQ ID NO: 144) |
| T032 | IFNGR2 transcript | VILISVGTFSLLSVLAGACFF (SEQ ID NO: 145) |
| | variant 1 | |
| | NM_001329128_1 | |
| T033 | IFNLR1 NM_170743_3 | FLVLPSLLILLLVIAAGGVIW (SEQ ID NO: 146) |
| T034 | IL1R1 transcript variant | HMIGICVTLTVIIVCSVFIYKIF (SEQ ID NO: 147) |
| | 2 NM_001288706_1 | |
| T035 | IL1RAP transcript | VLLVVILIVVYHVYWLEMVLF (SEQ ID NO: 148) |
| | variant 1 NM_002182_3 | |
| T036 | IL1RL1 transcript | IYCIIAVCSVFLMLINVLVII (SEQ ID NO: 149) |
| | variant 1 NM_016232.4 | |
| T037 | IL1RL2 NM_003854.2 | AYLIGGLIALVAVAVSVVYIY (SEQ ID NO: 150) |
| T038 | IL2RA transcript variant | VAVAGCVFLLISVLLLSGL (SEQ ID NO: 151) |
| | 1 NM_000417_2 | |
| T039 | IL2RB transcript variant | IPWLGHLLVGLSGAFGFIILVYLLI (SEQ ID NO: 152) |
| | 1 NM_000878_4 | |
| T040 | IL2RG NM_000206_2 | VVISVGSMGLIISLLCVYFWL (SEQ ID NO: 153) |
| T041 | IL3RA transcript variant | TSLLIALGTLLALVCVFVIC (SEQ ID NO: 154) |
| | 1 and 2 NM_002183_3 | |
| T042 | IL4R transcript variant 1 | LLLGVSVSCIVILAVCLLCYVSIT (SEQ ID NO: 155) |
| | NM_000418_3 | |
| T043 | IL5RA transcript variant | FVIVIMATICFILLILSLIC (SEQ ID NO: 156) |
| | 1 NM_000564_4 | |
| T044 | IL6R transcript variant 1 | TFLVAGGSLAFGTLLCIAIVL (SEQ ID NO: 157) |
| | NM_000565_3 | |
| T045 | IL6ST transcript variant | AIVVPVCLAFLLTTLLGVLFCF (SEQ ID NO: 158) |
| | 1 and 3 NM_002184_3 | |
| T046 | IL7RA NM_002185_3 | ILLTISILSFFSVALLVILACVL (SEQ ID NO: 159) |
| T047 | IL7RA Ins PPCL | ILLPPCLTISILSFFSVALLVILACVL (SEQ ID NO: 160) |
| | (interleukin 7 receptor) | |
| T048 | IL9R transcript variant 1 | GNTLVAVSIFLLLTGPTYLLF (SEQ ID NO: 161) |
| | NM_002186_2 | |
| T049 | IL10RA transcript | VIIFFAFVLLLSGALAYCLAL (SEQ ID NO: 162) |
| | variant 1 NM_001558_3 | |
| T050 | IL10RB NM_000628_4 | WMVAVILMASVFMVCLALLGCF (SEQ ID NO: 163) |
| T051 | IL11RA | SLGILSFLGLVAGALALGLWL (SEQ ID NO: 164) |
| | NM_001142784_2 | |
| T052 | IL12RB1 transcript | WLIFFASLGSFLSILLVGVLGYLGL (SEQ ID NO: 165) |
| | variant 1 and 4 | |
| | NM_005535_2 | |
| T053 | IL12RB2 transcript | WMAFVAPSICIAIIMVGIFST (SEQ ID NO: 166) |
| | variant 1 and 3 | |
| | NM_001559_2 | |
| T054 | IL13RA1 NM_001560_2 | LYITMLLIVPVIVAGAIIVLLLYL (SEQ ID NO: 167) |
| T055 | IL13RA2 NM_000640_2 | FWLPFGFILILVIFVTGLLL (SEQ ID NO: 168) |
| T056 | IL15RA transcript | VAISTSTVLLCGLSAVSLLACYL (SEQ ID NO: 169) |
| | variant 4 | |
| | NM_001256765_1 | |
| T057 | IL17RA NM_014339_6 | VYWFITGISILLVGSVILLIV (SEQ ID NO: 170) |
| T058 | IL17RB NM_018725_3 | LLLLSLLVATWVLVAGIYLMW (SEQ ID NO: 171) |
| T059 | IL17RC transcript | WALVWLACLLFAAALSLILLL (SEQ ID NO: 172) |
| | variant 1 NM_153460_3 | |
| T060 | IL17RD transcript | AVAITVPLVVISAFATLFTVM (SEQ ID NO: 173) |
| | variant 2 NM_017563_4 | |
| T061 | IL17RE transcript | LGLLILALLALLTLLGVVLAL (SEQ ID NO: 174) |
| | variant 1 NM_153480_1 | |
| T062 | IL18R1 transcript | GMIIAVLILVAVVCLVTVCVI (SEQ ID NO: 175) |
| | variant 1 NM_003855_3 | |
| T063 | IL18RAP NM_003853_3 | GVVLLYILLGTIGTLVAVLAA (SEQ ID NO: 176) |
| T064 | IL20RA transcript | IIFWYVLPISITVFLFSVMGY (SEQ ID NO: 177) |
| | variant 1 NM_014432_3 | |
| T065 | IL20RB NM_144717_3 | VLALFAFVGFMLILVVVPLFV (SEQ ID NO: 178) |
| T066 | IL21R transcript variant | GWNPHLLLLLLLVIVFIPAFW (SEQ ID NO: 179) |
| | 2 NM_181078_2 | |
| T067 | IL22RA1 NM_021258_3 | YSFSGAFLFSMGFLVAVLCYL (SEQ ID NO: 180) |
| T068 | IL23R NM_144701_2 | LLLGMIVFAVMLSILSLIGIF (SEQ ID NO: 181) |
| T069 | IL27RA NM_004843_3 | VLPGILFLWGLFLLGCGLSLA (SEQ ID NO: 182) |
| T070 | IL27RA NM_004843_3 | VLPGILCLWGLFLLGCGLSLA (SEQ ID NO: 183) |
| T071 | IL31RA transcript | IILITSLIGGGLLILIILTVAYGL (SEQ ID NO: 184) |
| | variant 1 NM_139017_5 | |
| T072 | LEPR transcript variant | AGLYVIVPVIISSSILLLGTLLI (SEQ ID NO: 185) |
| | 1 NM_002303_5 | |
| T073 | LIFR NM_001127671_1 | VGLIIAILIPVAVAVIVGVVTSILC (SEQ ID NO: 186) |
| T074 | MPL NM_005373_2 | ISLVTALHLVLGLSAVLGLLLL (SEQ ID NO: 187) |
| T075 | MPL NM_005373_2 | ISLVTALHLVLGLNAVLGLLLL (SEQ ID NO: 188) |
| T076 | OSMR transcript variant | LIHILLPMVFCVLLIMVMCYL (SEQ ID NO: 189) |
| | 4 NM_001323505_1 | |
| T077 | PRLR transcript variant | TTVWISVAVLSAVICLIIVWAVAL (SEQ ID NO: 190) |
| | 1 NM_000949_6 | |
| T078 | TNFRSF4 NM_003327_3 | VAAILGLGLVLGLLGPLAILL (SEQ ID NO: 191) |
| T079 | TNFRSF8 transcript | PVLDAGPVLFWVILVLVVVVGSSAFLLC (SEQ ID NO: 192) |
| | variant 1 NM_001243_4 | |
| T080 | TNFRSF9 NM_001561_5 | IISFFLALTSTALLFLLFFLTLRFSVV (SEQ ID NO: 193) |
| T081 | TNFRSF14 transcript | WWFLSGSLVIVIVCSTVGLII (SEQ ID NO: 194) |
| | variant 1 NM_003820_3 | |
| T082 | TNFRSF18 transcript | LGWLTVVLLAVAACVLLLTSA (SEQ ID NO: 195) |
| | variant 1 NM_004195_2 | |
| S036 | CD2 transcript variant 1 | TKRKKQRSRRNDEELETRAHRVATEERGRKPHQIPASTPQNPATSQHPPPPPGHRSQAPSHRPPPPGHRVQ |
| | NM_001328609_l | HQPQKRPPAPSGTQVHQQKGPPLPRPRVQPKPPHGAAENSLSPSSN (SEQ ID NO: 196) |
| S037 | CD3D transcript variant | GHETGRLSGAADTQALLRNDQVYQPLRDRDDAQYSHLGGNWARNK (SEQ ID NO: 197) |
| | 1 NM_000732_4 | |
| S038 | CD3E NM_000733_3 | KNRKAKAKPVTRGAGAGGRQRGQNKERPPPVPNPDYEPIRKGQRDLYSGLNQRRI (SEQ ID NO: 198) |
| S039 | CD3G NM_000073_2 | GQDGVRQSRASDKQTLLPNDQLYQPLKDREDDQYSHLQGNQLRRN (SEQ ID NO: 199) |
| S042 | CD4 transcript variant 1 | CVRCRHRRRQAERMSQIKRLLSEKKTCQCPHRFQKTCSPI (SEQ ID NO: 200) |
| | and 2 NM_000616_4 | |
| S043 | CD8A transcript variant | LYCNHRNRRRVCKCPRPVVKSGDKPSLSARYV (SEQ ID NO: 201) |
| | 1 NM_001768_6 | |
| S044 | CD8B transcript variant | RRRRARLRFMKQPQGEGISGTFVPQCLHGYYSNTTTSQKLLNPWILKT (SEQ ID NO: 202) |
| | 2 NM_172213_3 | |
| S045 | CD8B transcript variant | RRRRARLRFMKQLRLHPLEKCSRMDY (SEQ ID NO: 203) |
| | 3 NM_172101_3 | |
| S046 | CD8B transcript variant | RRRRARLRFMKQFYK (SEQ ID NO: 204) |
| | 5 NM_004931_4 | |
| S047 | CD27 NM_001242_4 | QRRKYRSNKGESPVEPAEPCRYSCPREEEGSTIPIQEDYRKPEPACSP (SEQ ID NO: 205) |
| S048 | mutated Delta Lek CD28 | RSKRSRLLHSDYMNMTPRRPGPTRKHYQAYAAARDFAAYRS (SEQ ID NO: 206) |
| | transcript variant 1 | |
| | NM_006139_3 | |
| S049 | CD28 transcript variant | RSKRSRLLHSDYMNMTPRRPGPTRKHYQPYAPPRDFAAYRS (SEQ ID NO: 207) |
| | 1 NM_006139_3 | |
| S050 | CD40 transcript variant | KKVAKKPTNKAPHPKQEPQEINFPDDLPGSNTAAPVQETLHGCQPVTQEDGKESRISVQERQ (SEQ ID |
| | 1 and 6 NM_001250_5 | NO: 208) |
| S051 | CD40 transcript variant | SESSEKVAKKPTNKAPHPKQEPQEINFPDDLPGSNTAAPVQETLHGCQPVTQEDGKESRISVQERQ (SEQ ID |
| | 5 NM_001322421_1 | NO: 209) |
| S052 | CD79A transcript | RKRWQNEKLGLDAGDEYEDENLYEGLNLDDCSMYEDISRGLQGTYQDVGSLNIGDVQLEKP (SEQ ID |
| | variant 1 NM_001783_3 | NQ: 210) |
| S053 | CD79B transcript | LDKDDSKAGMEEDHTYEGLDIDQTATYEDIVTLRTGEVKWSVGEHPGQE (SEQ ID NO: 211) |
| | variant 3 | |
| | NM_001039933_2 | |
| S054 | CRLF2 transcript variant | KLWRVKKFLIPSVPDPKSIFPGLFEIHQGNFQEWITDTQNVAHLHKMAGAEQESGPEEPLVVQLAKTEAESPR |
| | 1 NM_022148_3 | MLDPQTEEKEASGGSLQLPHQPLQGGDVVTIGGFTFVMNDRSYVAL (SEQ ID NO: 212) |
| S057 | CSF2RB NM_000395_2 | RFCGIYGYRLRRKWEEKIPNPSKSHLFQNGSAELWPPGSMSAFTSGSPPHQGPWGSRFPELEGVFPVGFGDS |
| | | EVSPLTIEDPKHVCDPPSGPDTTPAASDLPTEQPPSPQPGPPAASHTPEKQASSFDFNGPYLGPPHSRSLPDIL |
| | | GQPEPPQEGGSQKSPPPGSLEYLCLPAGGQVQLVPLAQAMGPGQAVEVERRPSQGAAGSPSLESGGGPAP |
| | | PALGPRVGGQDQKDSPVAIPMSSGDTEDPGVASGYVSSADLVFTPNSGASSVSLVPSLGLPSDQTPSLCPGL |
| | | ASGPPGAPGPVKSGFEGYVELPPIEGRSPRSPRNNPVPPEAKSPVLNPGERPADVSPTSPQPEGLLVLQQVGD |
| | | YCFLPGLGPGPLSLRSKPSSPGPGPEIKNLDQAFQVKKPPGQAVPQVPVIQLFKALKQQDYLSLPPWEVNKPG |
| | | EVC (SEQ ID NO: 213) |
| S058 | CSF2RA transcript | KRFLRIQRLFPPVPQIKDKLNDNHEVEDEIIWEEFTPEEGKGYREEVLTVKEIT (SEQ ID NO: 214) |
| | variant 7 and 8 | |
| | NM_001161529_1 | |
| S059 | CSF2RA transcript | KRFLRIQRLFPPVPQIKDKLNDNHEVEDEMGPQRHHRCGWNLYPTPGPSPGSGSSPRLGSESSL (SEQ ID |
| | variant 9 | NO: 215) |
| | NM_001161531_1 | |
| S062 | CSF3R transcript variant | SPNRKNPLWPSVPDPAHSSLGSWVPTIMEEDAFQLPGLGTPPITKLTVLEEDEKKPVPWESHNSSETCGLPTL |
| | 1 NM_000760_3 | VQTYVLQGDPRAVSTQPQSQSGTSDQVLYGQLLGSPTSPGPGHYLRCDSTQPLLAGLTPSPKSYENLWFQAS |
| | | PLGTLVTPAPSQEDDCVFGPLLNFPLLQGIRVHGMEALGSF (SEQ ID NO: 216) |
| S063 | CSF3R transcript variant | SPNRKNPLWPSVPDPAHSSLGSWVPTIMEELPGPRQGQWLGQTSEMSRALTPHPCVQDAFQLPGLGTPPI |
| | 3 NM_156039_3 | TKLTVLEEDEKKPVPWESHNSSETCGLPTLVQTYVLQGDPRAVSTQPQSQSGTSDQVLYGQLLGSPTSPGPG |
| | | HYLRCDSTQPLLAGLTPSPKSYENLWFQASPLGTLVTPAPSQEDDCVFGPLLNFPLLQGIRVHGMEALGSF |
| | | (SEQ ID NO: 217) |
| S064 | CSF3R transcript variant | SPNRKNPLWPSVPDPAHSSLGSWVPTIMEEDAFQLPGLGTPPITKLTVLEEDEKKPVPWESHNSSETCGLPTL |
| | 4 NM_172313_2 | VQTYVLQGDPRAVSTQPQSQSGTSDQAGPPRRSAYFKDQIMLHPAPPNGLLCLFPITSVL (SEQ ID NO: 218) |
| S069 | EPOR transcript variant | HRRALKQKIWPGIPSPESEFEGLFTTHKGNFQLWLYQNDGCLWWSPCTPFTEDPPASLEVLSERCWGTMQA |
| | 1 NM_000121_3 | VEPGTDDEGPLLEPVGSEHAQDTYLVLDKWLLPRNPPSEDLPGPGGSVDIVAMDEGSEASSCSSALASKPSPE |
| | | GASAASFEYTILDPSSQLLRPWTLCPELPPTPPHLKYLYLVVSDSGISTDYSSGDSQGAQGGLSDGPYSNPYENS |
| | | LIPAAEPLPPSYVACS (SEQ ID NO: 219) |
| S072 | EPOR transcript variant | HRRALKQKIWPGIPSPESEFEGLFTTHKGNFQLWLYQNDGCLWWSPCTPFTEDPPASLEVLSERCWGTMQA |
| | 1 NM_000121_3 | VEPGTDDEGPLLEPVGSEHAQDTYLVLDKWLLPRNPPSEDLPGPGGSVDIVAMDEGSEASSCSSALASKPSPE |
| | | GASAASFEYTILDPSSQLLRPWTLCPELPPTPPHLKFLFLVVSDSGISTDYSSGDSQGAQGGLSDGPYSNPYENS |
| | | LIPAAEPLPPSYVACS (SEQ ID NO: 220) |
| S074 | FCERIG NM_004106_1 | RLKIQVRKAAITSYEKSDGVYTGLSTRNQETYETLKHEKPPQ (SEQ ID NO: 221) |
| S075 | FCGR2C NM_201563_5 | CRKKRISANSTDPVKAAQFEPPGRQMIAIRKRQPEETNNDYETADGGYMTLNPRAPTDDDKNIYLTLPPNDH |
| | | VNSNN (SEQ ID NO: 222) |
| S076 | FCGRA2 transcript | CRKKRISANSTDPVKAAQFEPPGRQMIAIRKRQLEETNNDYETADGGYMTLNPRAPTDDDKNIYLTLPPNDH |
| | variant 1 | VNSNN (SEQ ID NO: 223) |
| | NM_001136219_1 | |
| S077 | GHR transcript variant 1 | KQQRIKMLILPPVPVPKIKGIDPDLLKEGKLEEVNTILAIHDSYKPEFHSDDSWVEFIELDIDEPDEKTEESDTDRL |
| | NM_000163_4 | LSSDHEKSHSNLGVKDGDSGRTSCCEPDILETDFNANDIHEGTSEVAQPQRLKGEADLLCLDQKNQNNSPYH |
| | | DACPATQQPSVIQAEKNKPQPLPTEGAESTHQAAHIQLSNPSSLSNIDFYAQVSDITPAGSVVLSPGQKNKAG |
| | | MSQCDMHPEMVSLCQENFLMDNAYFCEADAKKCIPVAPHIKVESHIQPSLNQEDIYITTESLTTAAGRPGTG |
| | | EHVPGSEMPVPDYTSIHIVQSPQGLILNATALPLPDKEFLSSCGYVSTDQLNKIMP (SEQ ID NO: 224) |
| S080 | ICOS NM_012092.3 | CWLTKKKYSSSVHDPNGEYMFMRAVNTAKKSRLTDVTL (SEQ ID NO: 225) |
| S081 | IFNAR1 NM_000629_2 | KVFLRCINYVFFPSLKPSSSIDEYFSEQPLKNLLLSTSEEQIEKCFIIENISTIATVEETNQTDEDHKKYSSQTSQDSG |
| | | NYSNEDESESKTSEELQQDFV (SEQ ID NO: 226) |
| S082 | IFNAR2 transcript | KWIGYICLRNSLPKVLNFHNFLAWPFPNLPPLEAMDMVEVIYINRKKKVWDYNYDDESDSDTEAAPRTSGG |
| | variant 1 NM_207585_2 | GYTMHGLTVRPLGQASATSTESQLIDPESEEEPDLPEVDVELPTMPKDSPQQLELLSGPCERRKSPLQDPFPEE |
| | | DYSSTEGSGGRITFNVDLNSVFLRVLDDEDSDDLEAPLMLSSHLEEMVDPEDPDNVQSNHLLASGEGTQPTF |
| | | PSPSSEGLWSEDAPSDQSDTSESDVDLGDGYIMR (SEQ ID NO: 227) |
| S083 | IFNAR2 transcript | KWIGYICLRNSLPKVLRQGLAKGWNAVAIHRCSHNALQSETPELKQSSCLSFPSSWDYKRASLCPSD (SEQ ID |
| | variant 2 NM_000874_4 | NO: 228) |
| S084 | IFNGR1 NM_000416_2 | CFYIKKINPLKEKSIILPKSLISVVRSATLETKPESKYVSLITSYQPFSLEKEVVCEEPLSPATVPGMHTEDNPGKVE |
| | | HTEELSSITEVVTTEENIPDVVPGSHLTPIERESSSPLSSNQSEPGSIALNSYHSRNCSESDHSRNGFDTDSSCLES |
| | | HSSLSDSEFPPNNKGEIKTEGQELITVIKAPTSFGYDKPHVLVDLLVDDSGKESLIGYRPTEDSKEFS (SEQ ID |
| | | NO: 229) |
| S085 | IFNGR2 transcript | LVLKYRGLIKYWFHTPPSIPLQIEEYLKDPTQPILEALDKDSSPKDDVWDSVSIISFPEKEQEDVLQTL (SEQ ID |
| | variant 1 | NQ: 230) |
| | NM_001329128_1 | |
| S086 | IFNLRI NM_170743_3 | KTLMGNPWFQRAKMPRALDFSGHTHPVATFQPSRPESVNDLFLCPQKELTRGVRPTPRVRAPATQQTRWK |
| | | KDLAEDEEEEDEEDTEDGVSFQPYIEPPSFLGQEHQAPGHSEAGGVDSGRPRAPLVPSEGSSAWDSSDRSW |
| | | ASTVDSSWDRAGSSGYLAEKGPGQGPGGDGHQESLPPPEFSKDSGFLEELPEDNLSSWATWGTLPPEPNLV |
| | | PGGPPVSLQTLTFCWESSPEEEEEARESEIEDSDAGSWGAESTQRTEDRGRTLGHYMAR (SEQ ID NO: 231) |
| S087 | IFNLRI transcript | KTLMGNPWFQRAKMPRALELTRGVRPTPRVRAPATQQTRWKKDLAEDEEEEDEEDTEDGVSFQPYIEPPSF |
| | variant 2 NM_173064_2 | LGQEHQAPGHSEAGGVDSGRPRAPLVPSEGSSAWDSSDRSWASTVDSSWDRAGSSGYLAEKGPGQGPGG |
| | | DGHQESLPPPEFSKDSGFLEELPEDNLSSWATWGTLPPEPNLVPGGPPVSLQTLTFCWESSPEEEEEARESEIE |
| | | DSDAGSWGAESTQRTEDRGRTLGHYMAR (SEQ ID NO: 232) |
| S098 | IL1R1 transcript variant | KIDIVLWYRDSCYDFLPIKVLPEVLEKQCGYKLFIYGRDDYVGEDIVEVINENVKKSRRLIIILVRETSGFSWLGGS |
| | 2 NM_001288706_1 | SEEQIAMYNALVQDGIKVVLLELEKIQDYEKMPESIKFIKQKHGAIRWSGDFTQGPQSAKTRFWKNVRYHMP |
| | | VQRRSPSSKHQLLSPATKEKLQREAHVPLG (SEQ ID NO: 233) |
| S099 | IL1R1 transcript variant | KIDIVLWYRDSCYDFLPIKASDGKTYDAYILYPKTVGEGSTSDCDIFVFKVLPEVLEKQCGYKLFIYGRDDYVGED |
| | 3 NM_001320978_1 | IVEVINENVKKSRRLIIILVRETSGFSWLGGSSEEQIAMYNALVQDGIKVVLLELEKIQDYEKMPESIKFIKQKHG |
| | | AIRWSGDFTQGPQSAKTRFWKNVRYHMPVQRRSPSSKHQLLSPATKEKLQREAHVPLG (SEQ ID NO: 234) |
| S100 | IL1RAP transcript | YRAHFGTDETILDGKEYDIYVSYARNAEEEEFVLLTLRGVLENEFGYKLCIFDRDSLPGGIVTDETLSFIQKSRRLL |
| | variant 1 NM_002182_3 | VVLSPNYVLQGTQALLELKAGLENMASRGNINVILVQYKAVKETKVKELKRAKTVLTVIKWKGEKSKYPQGRF |
| | | WKQLQVAMPVKKSPRRSSSDEQGLSYSSLKNV (SEQ ID NO: 235) |
| S101 | IL1RAP transcript | YRAHFGTDETILDGKEYDIYVSYARNAEEEEFVLLTLRGVLENEFGYKLCIFDRDSLPGGNTVEAVFDFIQRSRR |
| | variant 6 | MIVVLSPDYVTEKSISMLEFKLGVMCQNSIATKLIVVEYRPLEHPHPGILQLKESVSFVSWKGEKSKHSGSKFW |
| | NM_001167931_1 | KALRLALPLRSLSASSGWNESCSSQSDISLDHVQRRRSRLKEPPELQSSERAAGSPPAPGTMSKHRGKSSATCR |
| | | CCVTYCEGENHLRNKSRAEIHNQPQWETHLCKPVPQESETQWIQNGTRLEPPAPQISALALHHFTDLSNNN |
| | | DFYIL (SEQ ID NO: 236) |
| S102 | IL1RL1 transcript | LKMFWIEATLLWRDIAKPYKTRNDGKLYDAYVVYPRNYKSSTDGASRVEHFVHQILPDVLENKCGYTLCIYGR |
| | variant 1 NM_016232.4 | DMLPGEDVVTAVETNIRKSRRHIFILTPQITHNKEFAYEQEVALHCALIQNDAKVILIEMEALSELDMLQAEAL |
| | | QDSLQHLMKVQGTIKWREDHIANKRSLNSKFWKHVRYQMPVPSKIPRKASSLTPLAAQKQ (SEQ ID |
| | | NO: 237) |
| S103 | IL1RL2 NM_003854.2 | NIFKIDIVLWYRSAFHSTETIVDGKLYDAYVLYPKPHKESQRHAVDALVLNILPEVLERQCGYKLFIFGRDEFPG |
| | | QAVANVIDENVKLCRRLIVIVVPESLGFGLLKNLSEEQIAVYSALIQDGMKVILIELEKIEDYTVMPESIQYIKQKH |
| | | GAIRWHGDFTEQSQCMKTKFWKTVRYHMPPRRCRPFPPVQLLQHTPCYRTAGPELGSRRKKCTLTTG (SEQ |
| | | ID NO: 238) |
| S104 | IL2RA transcript variant | TWQRRQRKSRRTI (SEQ ID NO: 239) |
| | 1 NM_000417_2 | |
| S105 | IL2RB transcript variant | NCRNTGPWLKKVLKCNTPDPSKFFSQLSSEHGGDVQKWLSSPFPSSSFSPGGLAPEISPLEVLERDKVTQLLLQ |
| | 1 NM_000878_4 | QDKVPEPASLSSNHSLTSCFTNQGYFFFHLPDALEIEACQVYFTYDPYSEEDPDEGVAGAPTGSSPQPLQPLSG |
| | | EDDAYCTFPSRDDLLLFSPSLLGGPSPPSTAPGGSGAGEERMPPSLQERVPRDWDPQPLGPPTPGVPDLVDF |
| | | QPPPELVLREAGEEVPDAGPREGVSFPWSRPPGQGEFRALNARLPLNTDAYLSLQELQGQDPTHLV (SEQ ID |
| | | NO: 240) |
| S106 | IL2RG NM_000206_2 | ERTMPRIPTLKNLEDLVTEYHGNFSAWSGVSKGLAESLQPDYSERLCLVSEIPPKGGALGEGPGASPCNQHSP |
| | | YWAPPCYTLKPET (SEQ ID NO: 241) |
| S109 | IL3RA transcript variant | RRYLVMQRLFPRIPHMKDPIGDSFQNDKLVVWEAGKAGLEECLVTEVQVVQKT (SEQ ID NO: 242) |
| | 1 and 2 NM_002183_3 | |
| S110 | IL4R transcript variant 1 | KIKKEWWDQIPNPARSRLVAIIIQDAQGSQWEKRSRGQEPAKCPHWKNCLTKLLPCFLEHNMKRDEDPHKA |
| | NM_000418_3 | AKEMPFQGSGKSAWCPVEISKTVLWPESISVVRCVELFEAPVECEEEEEVEEEKGSFCASPESSRDDFQEGRE |
| | | GIVARLTESLFLDLLGEENGGFCQQDMGESCLLPPSGSTSAHMPWDEFPSAGPKEAPPWGKEQPLHLEPSPP |
| | | ASPTQSPDNLTCTETPLVIAGNPAYRSFSNSLSQSPCPRELGPDPLLARHLEEVEPEMPCVPQLSEPTTVPQPE |
| | | PETWEQILRRNVLQHGAAAAPVSAPTSGYQEFVHAVEQGGTQASAVVGLGPPGEAGYKAFSSLLASSAVSPE |
| | | KCGFGASSGEEGYKPFQDLIPGCPGDPAPVPVPLFTFGLDREPPRSPQSSHLPSSSPEHLGLEPGEKVEDMPKP |
| | | PLPQEQATDPLVDSLGSGIVYSALTCHLCGHLKQCHGQEDGGQTPVMASPCCGCCCGDRSSPPTTPLRAPDP |
| | | SPGGVPLEASLCPASLAPSGISEKSKSSSSFHPAPGNAQSSSQTPKIVNFVSVGPTYMRVS (SEQ ID NO: 243) |
| S113 | IL4R transcript variant 1 | KIKKEWWDQIPNPARSRLVAIIIQDAQGSQWEKRSRGQEPAKCPHWKNCLTKLLPCFLEHNMKRDEDPHKA |
| | NM_000418_3 | AKEMPFQGSGKSAWCPVEISKTVLWPESISVVRCVELFEAPVECEEEEEVEEEKGSFCASPESSRDDFQEGRE |
| | | GIVARLTESLFLDLLGEENGGFCQQDMGESCLLPPSGSTSAHMPWDEFPSAGPKEAPPWGKEQPLHLEPSPP |
| | | ASPTQSPDNLTCTETPLVIAGNPAYRSFSNSLSQSPCPRELGPDPLLARHLEEVEPEMPCVPQLSEPTTVPQPE |
| | | PETWEQILRRNVLQHGAAAAPVSAPTSGYQEFVHAVEQGGTQASAVVGLGPPGEAGYKAFSSLLASSAVSPE |
| | | KCGFGASSGEEGYKPFQDLIPGCPGDPAPVPVPLFTFGLDREPPRSPQSSHLPSSSPEHLGLEPGEKVEDMPKP |
| | | PLPQEQATDPLVDSLGSGIVFSALTCHLCGHLKQCHGQEDGGQTPVMASPCCGCCCGDRSSPPTTPLRAPDP |
| | | SPGGVPLEASLCPASLAPSGISEKSKSSSSFHPAPGNAQSSSQTPKIVNFVSVGPTYMRVS (SEQ ID NO: 244) |
| S115 | IL5RA transcript variant | KICHLWIKLFPPIPAPKSNIKDLFVTTNYEKAGSSETEIEVICYIEKPGVETLEDSVF (SEQ ID NO: 245) |
| | 1 NM_000564_4 | |
| S116 | IL6R transcript variant 1 | RFKKTWKLRALKEGKTSMHPPYSLGQLVPERPRPTPVLVPLISPPVSPSSLGSDNTSSHNRPDARDPRSPYDIS |
| | NM_000565_3 | NTDYFFPR (SEQ ID NO: 246) |
| S117 | IL6ST transcript variant | NKRDLIKKHIWPNVPDPSKSHIAQWSPHTPPRHNFNSKDQMYSDGNFTDVSVVEIEANDKKPFPEDLKSLDL |
| | 1 and 3 NM_002184_3 | FKKEKINTEGHSSGIGGSSCMSSSRPSISSSDENESSQNTSSTVQYSTVVHSGYRHQVPSVQVFSRSESTQPLLD |
| | | SEERPEDLQLVDHVDGGDGILPRQQYFKQNCSQHESSPDISHFERSKQVSSVNEEDFVRLKQQISDHISQSCG |
| | | SGQMKMFQEVSAADAFGPGTEGQVERFETVGMEAATDEGMPKSYLPQTVRQGGYMPQ (SEQ ID |
| | | NO: 247) |
| S120 | IL7RA Isoform 1 | WKKRIKPIVWPSLPDHKKTLEHLCKKPRKNLNVSFNPESFLDCQIHRVDDIQARDEVEGFLQDTFPQQLEESE |
| | NM_002185.4 | KQRLGGDVQSPNCPSEDVVITPESFGRDSSLTCLAGNVSACDAPILSSSRSLDCRESGKNGPHVYQDLLLSLGT |
| | | TNSTLPPPFSLQSGILTLNPVAQGQPILTSLGSNQEEAYVTMSSFYQNQ (SEQ ID NO: 248) |
| S121 | IL7RA Isoform 3 (C-term | WKKRIKPIVWPSLPDHKKTLEHLCKKPRKVSVFGA (SEQ ID NO: 249) |
| | deletion) (interleukin 7 | |
| | receptor) | |
| S126 | IL9R transcript variant 1 | KLSPRVKRIFYQNVPSPAMFFQPLYSVHNGNFQTWMGAHGAGVLLSQDCAGTPQGALEPCVQEATALLTC |
| | NM_002186_2 | GPARPWKSVALEEEQEGPGTRLPGNLSSEDVLPAGCTEWRVQTLAYLPQEDWAPTSLTRPAPPDSEGSRSSS |
| | | SSSSSNNNNYCALGCYGGWHLSALPGNTQSSGPIPALACGLSCDHQGLETQQGVAWVLAGHCQRPGLHED |
| | | LQGMLLPSVLSKARSWTF (SEQ ID NO: 250) |
| S129 | IL10RA transcript | QLYVRRRKKLPSVLLFKKPSPFIFISQRPSPETQDTIHPLDEEAFLKVSPELKNLDLHGSTDSGFGSTKPSLQTEEP |
| | variant 1 NM_001558_3 | QFLLPDPHPQADRTLGNREPPVLGDSCSSGSSNSTDSGICLQEPSLSPSTGPTWEQQVGSNSRGQDDSGIDL |
| | | VQNSEGRAGDTQGGSALGHHSPPEPEVPGEEDPAAVAFQGYLRQTRCAEEKATKTGCLEEESPLTDGLGPKF |
| | | GRCLVDEAGLHPPALAKGYLKQDPLEMTLASSGAPTGQWNQPTEEWSLLALSSCSDLGISDWSFAHDLAPL |
| | | GCVAAPGGLLGSFNSDLVTLPLISSLQSSE (SEQ ID NO: 251) |
| S130 | IL10RB NM_000628_4 | ALLWCVYKKTKYAFSPRNSLPQHLKEFLGHPHHNTLLFFSFPLSDENDVFDKLSVIAEDSESGKQNPGDSCSLG |
| | | TPPGQGPQS (SEQ ID NO: 252) |
| S135 | IL11RA | RLRRGGKDGSPKPGFLASVIPVDRRPGAPNL (SEQ ID NO: 253) |
| | NM_001142784 2 | |
| S136 | IL12RB1 transcript | NRAARHLCPPLPTPCASSAIEFPGGKETWQWINPVDFQEEASLQEALVVEMSWDKGERTEPLEKTELPEGAP |
| | variant 1 and 4 | ELALDTELSLEDGDRCKAKM (SEQ ID NO: 254) |
| | NM_005535_2 | |
| S137 | IL12RB1 transcript | NRAARHLCPPLPTPCASSAIEFPGGKETWQWINPVDFQEEASLQEALVVEMSWDKGERTEPLEKTELPEGAP |
| | variant 3 | ELALDTELSLEDGDRCDR (SEQ ID NO: 255) |
| | NM_001290023_1 | |
| S138 | IL12RB2 transcript | HYFQQKVFVLLAALRPQWCSREIPDPANSTCAKKYPIAEEKTQLPLDRLLIDWPTPEDPEPLVISEVLHQVTPVF |
| | variant 1 and 3 | RHPPCSNWPQREKGIQGHQASEKDMMHSASSPPPPRALQAESRQLVDLYKVLESRGSDPKPENPACPWTV |
| | NM_001559_2 | LPAGDLPTHDGYLPSNIDDLPSHEAPLADSLEELEPQHISLSVFPSSSLHPLTFSCGDKLTLDQLKMRCDSLML |
| | | (SEQ ID NO: 256) |
| S141 | IL13RA1 NM_001560_2 | KRLKIIIFPPIPDPGKIFKEMFGDQNDDTLHWKKYDIYEKQTKEETDSVVLIENLKKASQ (SEQ ID NO: 257) |
| S142 | IL13RA2 NM_000640_2 | RKPNTYPKMIPEFFCDT (SEQ ID NO: 258) |
| S143 | IL15RA transcript | KSRQTPPLASVEMEAMEALPVTWGTSSRDEDLENCSHHL (SEQ ID NO: 259) |
| | variant 4 | |
| | NM_001256765_1 | |
| S144 | IL17RA NM_014339_6 | CMTWRLAGPGSEKYSDDTKYTDGLPAADLIPPPLKPRKVWIIYSADHPLYVDVVLKFAQFLLTACGTEVALDLL |
| | | EEQAISEAGVMTWVGRQKQEMVESNSKIIVLCSRGTRAKWQALLGRGAPVRLRCDHGKPVGDLFTAAMN |
| | | MILPDFKRPACFGTYVVCYFSEVSCDGDVPDLFGAAPRYPLMDRFEEVYFRIQDLEMFQPGRMHRVGELSG |
| | | DNYLRSPGGRQLRAALDRFRDWQVRCPDWFECENLYSADDQDAPSLDEEVFEEPLLPPGTGIVKRAPLVREP |
| | | GSQACLAIDPLVGEEGGAAVAKLEPHLQPRGQPAPQPLHTLVLAAEEGALVAAVEPGPLADGAAVRLALAGE |
| | | GEACPLLGSPGAGRNSVLFLPVDPEDSPLGSSTPMASPDLLPEDVREHLEGLMLSLFEQSLSCQAQGGCSRPA |
| | | MVLTDPHTPYEEEQRQSVQSDQGYISRSSPQPPEGLTEMEEEEEEEQDPGKPALPLSPEDLESLRSLQRQLLFR |
| | | QLQKNSGWDTMGSESEGPSA (SEQ ID NO: 260) |
| S145 | IL17RB NM_018725_3 | RHERIKKTSFSTTTLLPPIKVLVVYPSEICFHHTICYFTEFLQNHCRSEVILEKWQKKKIAEMGPVQWLATQKKA |
| | | ADKVVFLLSNDVNSVCDGTCGKSEGSPSENSQDLFPLAFNLFCSDLRSQIHLHKYVVVYFREIDTKDDYNALSV |
| | | CPKYHLMKDATAFCAELLHVKQQVSAGKRSQACHDGCCSL (SEQ ID NO: 261) |
| S146 | IL17RC transcript | KKDHAKGWLRLLKQDVRSGAAARGRAALLLYSADDSGFERLVGALASALCQLPLRVAVDLWSRRELSAQGP |
| | variant 1 NM_153460_3 | VAWFHAQRRQTLQEGGVVVLLFSPGAVALCSEWLQDGVSGPGAHGPHDAFRASLSCVLPDFLQGRAPGSY |
| | | VGACFDRLLHPDAVPALFRTVPVFTLPSQLPDFLGALQQPRAPRSGRLQERAEQVSRALQPALDSYFHPPGTP |
| | | APGRGVGPGAGPGAGDGT (SEQ ID NO: 262) |
| S147 | IL17RC transcript | KKDHAKAAARGRAALLLYSADDSGFERLVGALASALCQLPLRVAVDLWSRRELSAQGPVAWFHAQRRQTLQ |
| | variant 4 | EGGVVVLLFSPGAVALCSEWLQDGVSGPGAHGPHDAFRASLSCVLPDFLQGRAPGSYVGACFDRLLHPDAV |
| | NM_001203263_1 | PALFRTVPVFTLPSQLPDFLGALQQPRAPRSGRLQERAEQVSRALQPALDSYFHPPGTPAPGRGVGPGAGPG |
| | | AGDGT (SEQ ID NO: 263) |
| S148 | IL17RD transcript | CRKKQQENIYSHLDEESSESSTYTAALPRERLRPRPKVFLCYSSKDGQNHMNVVQCFAYFLQDFCGCEVALDL |
| | variant 2 NM_017563_4 | WEDFSLCREGQREWVIQKIHESQFIIVVCSKGMKYFVDKKNYKHKGGGRGSGKGELFLVAVSAIAEKLRQAK |
| | | QSSSAALSKFIAVYFDYSCEGDVPGILDLSTKYRLMDNLPQLCSHLHSRDHGLQEPGQHTRQGSRRNYFRSKS |
| | | GRSLYVAICNMHQFIDEEPDWFEKQFVPFHPPPLRYREPVLEKFDSGLVLNDVMCKPGPESDFCLKVEAAVL |
| | | GATGPADSQHESQHGGLDQDGEARPALDGSAALQPLLHTVKAGSPSDMPRDSGIYDSSVPSSELSLPLMEG |
| | | LSTDQTETSSLTESVSSSSGLGEEEPPALPSKLLSSGSCKADLGCRSYTDELHAVAPL (SEQ ID NO: 264) |
| S149 | IL17RE transcript | TCRRPQSGPGPARPVLLLHAADSEAQRRLVGALAELLRAALGGGRDVIVDLWEGRHVARVGPLPWLWAAR |
| | variant 1 NM_153480_1 | TRVAREQGTVLLLWSGADLRPVSGPDPRAAPLLALLHAAPRPLLLLAYFSRLCAKGDIPPPLRALPRYRLLRDLP |
| | | RLLRALDARPFAEATSWGRLGARQRRQSRLELCSRLEREAARLADLG (SEQ ID NO: 265) |
| S154 | IL18R1 transcript | YRVDLVLFYRHLTRRDETLTDGKTYDAFVSYLKECRPENGEEHTFAVEILPRVLEKHFGYKLCIFERDVVPGGAV |
| | variant 1 NM_003855_3 | VDEIHSLIEKSRRLIIVLSKSYMSNEVRYELESGLHEALVERKIKIILIEFTPVTDFTFLPQSLKLLKSHRVLKWKADK |
| | | SLSYNSRFWKNLLYLMPAKTVKPGRDEPEVLPVLSES (SEQ ID NO: 266) |
| S155 | IL18RAP NM_003853_3 | SALLYRHWIEIVLLYRTYQSKDQTLGDKKDFDAFVSYAKWSSFPSEATSSLSEEHLALSLFPDVLENKYGYSLCLL |
| | | ERDVAPGGVYAEDIVSIIKRSRRGIFILSPNYVNGPSIFELQAAVNLALDDQTLKLILIKFCYFQEPESLPHLVKKAL |
| | | RVLPTVTWRGLKSVPPNSRFWAKMRYHMPVKNSQGFTWNQLRITSRIFQWKGLSRTETTGRSSQPKEW |
| | | (SEQ ID NO: 267) |
| S156 | IL20RA transcript | SIYRYIHVGKEKHPANLILIYGNEFDKRFFVPAEKIVINFITLNISDDSKISHQDMSLLGKSSDVSSLNDPQPSGNL |
| | variant 1 NM_014432_3 | RPPQEEEEVKHLGYASHLMEIFCDSEENTEGTSLTQQESLSRTIPPDKTVIEYEYDVRTTDICAGPEEQELSLQEE |
| | | VSTQGTLLESQAALAVLGPQTLQYSYTPQLQDLDPLAQEHTDSEEGPEEEPSTTLVDWDPQTGRLCIPSLSSFD |
| | | QDSEGCEPSEGDGLGEEGLLSRLYEEPAPDRPPGENETYLMQFMEEWGLYVQMEN (SEQ ID NO: 268) |
| S157 | IL20RB NM_144717_3 | WKMGRLLQYSCCPVVVLPDTLKITNSPQKLISCRREEVDACATAVMSPEELLRAWIS (SEQ ID NO: 269) |
| S158 | IL21R transcript variant | SLKTHPLWRLWKKIWAVPSPERFFMPLYKGCSGDFKKWVGAPFTGSSLELGPWSPEVPSTLEVYSCHPPRSP |
| | 2 NM_181078_2 | AKRLQLTELQEPAELVESDGVPKPSFWPTAQNSGGSAYSEERDRPYGLVSIDTVTVLDAEGPCTWPCSCEDD |
| | | GYPALDLDAGLEPSPGLEDPLLDAGTTVLSCGCVSAGSPGLGGPLGSLLDRLKPPLADGEDWAGGLPWGGRS |
| | | PGGVSESEAGSPLAGLDMDTFDSGFVGSDCSSPVECDFTSPGDEGPPRSYLRQWVVIPPPLSSPGPQAS |
| | | (SEQ ID NO: 270) |
| S161 | IL22RA1 NM_021258_3 | SYRYVTKPPAPPNSLNVQRVLTFQPLRFIQEHVLIPVFDLSGPSSLAQPVQYSQIRVSGPREPAGAPQRHSLSEI |
| | | TYLGQPDISILQPSNVPPPQILSPLSYAPNAAPEVGPPSYAPQVTPEAQFPFYAPQAISKVQPSSYAPQATPDS |
| | | WPPSYGVCMEGSGKDSPTGTLSSPKHLRPKGQLQKEPPAGSCMLGGLSLQEVTSLAMEESQEAKSLHQPLGI |
| | | CTDRTSDPNVLHSGEEGTPQYLKGQLPLLSSVQIEGHPMSLPLQPPSRPCSPSDQGPSPWGLLESLVCPKDEA |
| | | KSPAPETSDLEQPTELDSLFRGLALTVQWES (SEQ ID NO: 271) |
| S165 | IL23R NM_144701_2 | NRSFRTGIKRRILLLIPKWLYEDIPNMKNSNVVKMLQENSELMNNNSSEQVLYVDPMITEIKEIFIPEHKPTDYK |
| | | KENTGPLETRDYPQNSLFDNTTVVYIPDLNTGYKPQISNFLPEGSHLSNNNEITSLTLKPPVDSLDSGNNPRLQ |
| | | KHPNFAFSVSSVNSLSNTIFLGELSLILNQGECSSPDIQNSVEEETTMLLENDSPSETIPEQTLLPDEFVSCLGIVN |
| | | EELPSINTYFPQNILESHFNRISLLEK (SEQ ID NO: 272) |
| S168 | IL27RA NM_004843_3 | TSGRCYHLRHKVLPRWVWEKVPDPANSSSGQPHMEQVPEAQPLGDLPILEVEEMEPPPVMESSQPAQATA |
| | | PLDSGYEKHFLPTPEELGLLGPPRPQVLA (SEQ ID NO: 273) |
| S169 | IL27RA NM_004843_3 | TSWVWEKVPDPANSSSGQPHMEQVPEAQPLGDLPILEVEEMEPPPVMESSQPAQATAPLDSGYEKHFLPT |
| | | PEELGLLGPPRPQVLA (SEQ ID NO: 274) |
| S170 | IL31RA transcript | KKPNKLTHLCWPTVPNPAESSIATWHGDDFKDKLNLKESDDSVNTEDRILKPCSTPSDKLVIDKLVVNFGNVL |
| | variant 1 NM_139017_5 | QEIFTDEARTGQENNLGGEKNGYVTCPFRPDCPLGKSFEELPVSPEIPPRKSQYLRSRMPEGTRPEAKEQLLFS |
| | | GQSLVPDHLCEEGAPNPYLKNSVTAREFLVSEKLPEHTKGEV (SEQ ID NO: 275) |
| S171 | IL31RA transcript | KKPNKLTHLCWPTVPNPAESSIATWHGDDFKDKLNLKESDDSVNTEDRILKPCSTPSDKLVIDKLVVNFGNVL |
| | variant 4 | QEIFTDEARTGQENNLGGEKNGTRILSSCPTSI (SEQ ID NO: 276) |
| | NM_001242638_1 | |
| S174 | LEPR transcript variant | SHQRMKKLFWEDVPNPKNCSWAQGLNFQKPETFEHLFIKHTASVTCGPLLLEPETISEDISVDTSWKNKDEM |
| | 1 NM_002303_5 | MPTTVVSLLSTTDLEKGSVCISDQFNSVNFSEAEGTEVTYEDESQRQPFVKYATLISNSKPSETGEEQGLINSSV |
| | | TKCFSSKNSPLKDSFSNSSWEIEAQAFFILSDQHPNIISPHLTFSEGLDELLKLEGNFPEENNDKKSIYYLGVTSIK |
| | | KRESGVLLTDKSRVSCPFPAPCLFTDIRVLQDSCSHFVENNINLGTSSKKTFASYMPQFQTCSTQTHKIMENK |
| | | MCDLTV (SEQ ID NO: 277) |
| S175 | LEPR transcript variant | SHQRMKKLFWEDVPNPKNCSWAQGLNFQKMLEGSMFVKSHHHSLISSTQGHKHCGRPQGPLHRKTRDLC |
| | 2 NM_001003680_3 | SLVYLLTLPPLLSYDPAKSPSVRNTQE (SEQ ID NO: 278) |
| S176 | LEPR transcript variant | SHQRMKKLFWEDVPNPKNCSWAQGLNFQKRTDIL (SEQ ID NO: 279) |
| | 3 NM_001003679_3 | |
| S177 | LEPR transcript variant | SHQRMKKLFWEDVPNPKNCSWAQGLNFQKKMPGTKELLGGGWLT (SEQ ID NO: 280) |
| | 5 NM_001198688_1 | |
| S180 | LIFR NM_001127671_1 | YRKREWIKETFYPDIPNPENCKALQFQKSVCEGSSALKTLEMNPCTPNNVEVLETRSAFPKIEDTEIISPVAERP |
| | | EDRSDAEPENHVVVSYCPPIIEEEIPNPAADEAGGTAQVIYIDVQSMYQPQAKPEEEQENDPVGGAGYKPQ |
| | | MHLPINSTVEDIAAEEDLDKTAGYRPQANVNTWNLVSPDSPRSIDSNSEIVSFGSPCSINSRQFLIPPKDEDSPK |
| | | SNGGGWSFTNFFQNKPND (SEQ ID NO: 281) |
| S183 | LMP1 NC_007605_1 | YYHGQRHSDEHHHDDSLPHPQQATDDSGHESDSNSNEGRHHLLVSGAGDGPPLCSQNLGAPGGGPDNGP |
| | | QDPDNTDDNGPQDPDNTDDNGPHDPLPQDPDNTDDNGPQDPDNTDDNGPHDPLPHSPSDSAGNDGGP |
| | | PQLTEEVENKGGDQGPPLMTDGGGGHSHDSGHGGGDPHLPTLLLGSSGSGGDDDDPHGPVQLSYYD |
| | | (SEQ ID NO: 282) |
| S186 | MPL NM_005373_2 | RWQFPAHYRRLRHALWPSLPDLHRVLGQYLRDTAALSPPKATVSDTCEEVEPSLLEILPKSSERTPLPLCSSQA |
| | | QMDYRRLQPSCLGTMPLSVCPPMAESGSCCTTHIANHSYLPLSYWQQP (SEQ ID NO: 283) |
| S189 | MYD88 transcript | MAAGGPGAGSAAPVSSTSSLPLAALNMRVRRRLSLFLNVRTQVAADWTALAEEMDFEYLEIRQLETQADPT |
| | variant 1 | GRLLDAWQGRPGASVGRLLELLTKLGRDDVLLELGPSIEEDCQKYILKQQQEEAEKPLQVAAVDSSVPRTAEL |
| | NM_001172567_1 | AGITTLDDPLGHMPERFDAFICYCPSDIQFVQEMIRQLEQTNYRLKLCVSDRDVLPGTCVWSIASELIEKRLAR |
| | | RPRGGCRRMVVVVSDDYLQSKECDFQTKFALSLSPGAHQKRLIPIKYKAMKKEFPSILRFITVCDYTNPCTKSW |
| | | FWTRLAKALSLP (SEQ ID NO: 284) |
| S190 | MYD88 transcript | MAAGGPGAGSAAPVSSTSSLPLAALNMRVRRRLSLFLNVRTQVAADWTALAEEMDFEYLEIRQLETQADPT |
| | variant 2 NM_002468_4 | GRLLDAWQGRPGASVGRLLELLTKLGRDDVLLELGPSIEEDCQKYILKQQQEEAEKPLQVAAVDSSVPRTAEL |
| | | AGITTLDDPLGHMPERFDAFICYCPSDIQFVQEMIRQLEQTNYRLKLCVSDRDVLPGTCVWSIASELIEKRCRR |
| | | MVVVVSDDYLQSKECDFQTKFALSLSPGAHQKRLIPIKYKAMKKEFPSILRFITVCDYTNPCTKSWFWTRLAKA |
| | | LSLP (SEQ ID NO: 285) |
| S191 | MYD88 transcript | MAAGGPGAGSAAPVSSTSSLPLAALNMRVRRRLSLFLNVRTQVAADWTALAEEMDFEYLEIRQLETQADPT |
| | variant 3 | GRLLDAWQGRPGASVGRLLELLTKLGRDDVLLELGPSIGHMPERFDAFICYCPSDIQFVQEMIRQLEQTNYRL |
| | NM_001172568_1 | KLCVSDRDVLPGTCVWSIASELIEKRCRRMVVVVSDDYLQSKECDFQTKFALSLSPGAHQKRLIPIKYKAMKKE |
| | | FPSILRFITVCDYTNPCTKSWFWTRLAKALSLP (SEQ ID NO: 286) |
| S192 | MYD88 transcript | MAAGGPGAGSAAPVSSTSSLPLAALNMRVRRRLSLFLNVRTQVAADWTALAEEMDFEYLEIRQLETQADPT |
| | variant 4 | GRLLDAWQGRPGASVGRLLELLTKLGRDDVLLELGPSIEEDCQKYILKQQQEEAEKPLQVAAVDSSVPRTAEL |
| | NM_001172569_1 | AGITTLDDPLGAAGWWWLSLMITCRARNVTSRPNLHSASLQVPIRSD (SEQ ID NO: 287) |
| S193 | MYD88 transcript | MAAGGPGAGSAAPVSSTSSLPLAALNMRVRRRLSLFLNVRTQVAADWTALAEEMDFEYLEIRQLETQADPT |
| | variant 5 | GRLLDAWQGRPGASVGRLLELLTKLGRDDVLLELGPSIGAAGWWWLSLMITCRARNVTSRPNLHSASLQVPI |
| | NM_001172566_1 | RSD (SEQ ID NO: 288) |
| S194 | MYD88 transcript | MAAGGPGAGSAAPVSSTSSLPLAALNMRVRRRLSLFLNVRTQVAADWTALAEEMDFEYLEIRQLETQADPT |
| | variant 1 | GRLLDAWQGRPGASVGRLLELLTKLGRDDVLLELGPSIEEDCQKYILKQQQEEAEKPLQVAAVDSSVPRTAEL |
| | NM_001172567_1 | AGITTLDDPLGHMPERFDAFICYCPSDI (SEQ ID NO: 289) |
| S195 | MYD88 transcript | MAAGGPGAGSAAPVSSTSSLPLAALNMRVRRRLSLFLNVRTQVAADWTALAEEMDFEYLEIRQLETQADPT |
| | variant 3 | GRLLDAWQGRPGASVGRLLELLTKLGRDDVLLELGPSIGHMPERFDAFICYCPSDI (SEQ ID NO: 290) |
| | NM_001172568_1 | |
| S196 | MYD88 transcript | MAAGGPGAGSAAPVSSTSSLPLAALNMRVRRRLSLFLNVRTQVAADWTALAEEMDFEYLEIRQLETQADPT |
| | variant 1 | GRLLDAWQGRPGASVGRLLELLTKLGRDDVLLELGPSIEEDCQKYILKQQQEEAEKPLQVAAVDSSVPRTAEL |
| | NM_001172567_1 | AGITTLDDPLGHMPERFDAFICYCPSDIQFVQEMIRQLEQTNYRLKLCVSDRDVLPGTCVWSIASELIEKRLAR |
| | | RPRGGCRRMVVVVSDDYLQSKECDFQTKFALSLSPGAHQKRPIPIKYKAMKKEFPSILRFITVCDYTNPCTKSW |
| | | FWTRLAKALSLP (SEQ ID NO: 291) |
| S197 | MYD88 transcript | MAAGGPGAGSAAPVSSTSSLPLAALNMRVRRRLSLFLNVRTQVAADWTALAEEMDFEYLEIRQLETQADPT |
| | variant 2 NM_002468_4 | GRLLDAWQGRPGASVGRLLELLTKLGRDDVLLELGPSIEEDCQKYILKQQQEEAEKPLQVAAVDSSVPRTAEL |
| | | AGITTLDDPLGHMPERFDAFICYCPSDIQFVQEMIRQLEQTNYRLKLCVSDRDVLPGTCVWSIASELIEKRCRR |
| | | MVVVVSDDYLQSKECDFQTKFALSLSPGAHQKRPIPIKYKAMKKEFPSILRFITVCDYTNPCTKSWFWTRLAKA |
| | | LSLP (SEQ ID NO: 292) |
| S198 | MYD88 transcript | MAAGGPGAGSAAPVSSTSSLPLAALNMRVRRRLSLFLNVRTQVAADWTALAEEMDFEYLEIRQLETQADPT |
| | variant 3 | GRLLDAWQGRPGASVGRLLELLTKLGRDDVLLELGPSIGHMPERFDAFICYCPSDIQFVQEMIRQLEQTNYRL |
| | NM_001172568_1 | KLCVSDRDVLPGTCVWSIASELIEKRCRRMVVVVSDDYLQSKECDFQTKFALSLSPGAHQKRPIPIKYKAMKKE |
| | | FPSILRFITVCDYTNPCTKSWFWTRLAKALSLP (SEQ ID NO: 293) |
| S199 | OSMR transcript variant | KSQWIKETCYPDIPDPYKSSILSLIKFKENPHLIIMNVSDCIPDAIEVVSKPEGTKIQFLGTRKSLTETELTKPNYLYL |
| | 4 NM_001323505_1 | LPTEKNHSGPGPCICFENLTYNQAASDSGSCGHVPVSPKAPSMLGLMTSPENVLKALEKNYMNSLGEIPAGE |
| | | TSLNYVSQLASPMFGDKDSLPTNPVEAPHCSEYKMQMAVSLRLALPPPTENSSLSSITLLDPGEHYC (SEQ ID |
| | | NO: 294) |
| S202 | PRLR transcript variant | KGYSMVTCIFPPVPGPKIKGFDAHLLEKGKSEELLSALGCQDFPPTSDYEDLLVEYLEVDDSEDQHLMSVHSKE |
| | 1 NM_000949_6 | HPSQGMKPTYLDPDTDSGRGSCDSPSLLSEKCEEPQANPSTFYDPEVIEKPENPETTHTWDPQCISMEGKIPY |
| | | FHAGGSKCSTWPLPQPSQHNPRSSYHNITDVCELAVGPAGAPATLLNEAGKDALKSSQTIKSREEGKATQQR |
| | | EVESFHSETDQDTPWLLPQEKTPFGSAKPLDYVEIHKVNKDGALSLLPKQRENSGKPKKPGTPENNKEYAKVS |
| | | GVMDNNILVLVPDPHAKNVACFEESAKEAPPSLEQNQAEKALANFTATSSKCRLQLGGLDYLDPACFTHSFH |
| | | (SEQ ID NO: 295) |
| S211 | TNFRSF4 NM_003327_3 | ALYLLRRDQRLPPDAHKPPGGGSFRTPIQEEQADAHSTLAKI (SEQ ID NO: 296) |
| S212 | TNFRSF8 transcript | HRRACRKRIRQKLHLCYPVQTSQPKLELVDSRPRRSSTQLRSGASVTEPVAEERGLMSQPLMETCHSVGAAYL |
| | variant 1 NM_001243_4 | ESLPLQDASPAGGPSSPRDLPEPRVSTEHTNNKIEKIYIMKADTVIVGTVKAELPEGRGLAGPAEPELEEELEAD |
| | | HTPHYPEQETEPPLGSCSDVMLSVEEEGKEDPLPTAASGK (SEQ ID NO: 297) |
| S213 | TNFRSF9 NM_001561_5 | KRGRKKLLYIFKQPFMRPVQTTQEEDGCSCRFPEEEEGGCEL (SEQ ID NO: 298) |
| S214 | TNFRSF14 transcript | CVKRRKPRGDVVKVIVSVQRKRQEAEGEATVIEALQAPPDVTTVAVEETIPSFTGRSPNH (SEQ ID NO: 299) |
| | variant 1 NM_003820_3 | |
| S215 | TNFRSF18 transcript | QLGLHIWQLRSQCMWPRETQLLLEVPPSTEDARSCQFPEEERGERSAEEKGRLGDLWV (SEQ ID NO: 300) |
| | variant 1 NM_004195_2 | |
| S216 | TNFRSF18 transcript | QLGLHIWQLRKTQLLLEVPPSTEDARSCQFPEEERGERSAEEKGRLGDLWV (SEQ ID NO: 301) |
| | variant | |
| | 3_NM_148902_1 | |
| X001 | Linker | GSGGSEGGGSEGGAATAGSGSGS (SEQ ID NO: 302) |

What is claimed is:

1. A cell formulation, comprising modified lymphocytes in a delivery solution, wherein the modified lymphocytes have one or more replication incompetent recombinant retroviral particles (RIPs) associated with their surfaces, and wherein the modified lymphocytes comprise T cells,
wherein the RIPs comprise a polynucleotide encoding a a chimeric antigen receptor (CAR), wherein the lymphocytes comprise T cells comprising CD4+ cells and CD8+ cells,
wherein the RIPs comprise a polypeptide capable of binding to CD3 associated with the surface of the RIPs,
wherein at least 50% of the T cells in the cell formulation are surface CD3−, and
wherein at least 5% of the modified lymphocytes are in cell aggregates.

2. Use of replication incompetent recombinant retroviral particles in the manufacture of a kit for administering a cell formulation to a subject, wherein the use of the kit comprises:
a) contacting blood cells comprising lymphocytes ex vivo in a reaction mixture comprising a T cell activation element and the replication incompetent recombinant retroviral particles (RIPs), wherein the RIPs comprise a polynucleotide encoding a first polypeptide comprising a chimeric antigen receptor (CAR),
wherein the lymphocytes comprise T cells comprising CD4+ cells and CD8+ cells,
wherein said contacting facilitates association of the lymphocytes with the RIPs, and
wherein the RIPs modify the T cells to form a population of modified lymphocytes comprising modified T cells;
b) forming a cell formulation by suspending the population of modified lymphocytes in a delivery solution; and
c) administering the cell formulation to a subject through subcutaneous administration,
wherein at the time of the forming and/or the administering at least 5% of the modified T cells are in cell aggregates,
wherein at the time of the forming and/or the administering at least 50% of the modified T cells in the cell formulation are surface CD3−, and/or
wherein the modified T cells in the cell formulation are capable of producing a persistent population of genetically modified lymphocytes expressing the first polypeptide comprising the CAR, wherein the persistent population of genetically modified lymphocytes is capable of persisting in the subject for at least 21 days after administration.

3. The cell formulation of claim 1, or the use of claim 2, wherein at least 10% of the CD4+ cells and/or CD8+ cells are in cell aggregates in the formulation.

4. The cell formulation of claim 1, or the use of claim 2, wherein at least 50% of the CD4+ cells and/or CD8+ cells in the cell formulation are surface CD3−.

5. The cell formulation of claim 1, or the use of claim 2, wherein at least 90% of the CD4+ cells and/or CD8+ cells in the cell formulation are surface CD3−.

6. The cell formulation of or the use of claim 3, wherein the cell aggregates comprise 5 to 500 modified lymphocytes.

7. The cell formulation of claim 1, or the use of claim 2, wherein the cell aggregates are greater than 40 μm in diameter.

8. The cell formulation of claim 1, or the use of claim 2, wherein the cell formulation comprises between 3×10⁴ and 3×10⁹ modified lymphocytes.

9. The cell formulation of claim 1, or the use of claim 2, wherein the cell formulation has a volume between 0.5 ml and 20 ml and is contained within a syringe.

10. The use of claim 2, wherein the use further comprises collecting blood comprising the lymphocytes contacted in the reaction mixture from the subject before the contacting.

11. The use of claim 10, wherein between 5 ml and 50 ml of blood is collected from the subject.

12. The use of claim 2, wherein the reaction mixture has a volume of between 5 ml and 30 ml.

13. The use of claim 2, wherein the administered modified lymphocytes in the cell formulation produce a persisting population of genetically modified lymphocytes expressing the first polypeptide comprising the CAR, wherein the persisting population of genetically modified lymphocytes persists in the subject for at least 21 days after administration, and wherein the persisting population of genetically modified lymphocytes comprises genetically modified T cells.

14. The use of claim 2, wherein at least 1×10⁵ of the administered modified lymphocytes in the cell formulation or their progeny remain localized subcutaneously for at least 14 days.

15. The cell formulation of claim 1, wherein at least 10% of the modified lymphocytes are in cell aggregates, and wherein the cell formulation is in a syringe and has a volume of between 2 ml and 7 ml.

16. The cell formulation of claim 1, or the use of claim 2, wherein the cell formulation further comprises neutrophils.

17. The cell formulation of claim 1, or the use of claim 2, wherein the cell formulation comprises all types of nucleated blood cells.

18. The cell formulation of claim 1, or the use of claim 2, wherein the modified lymphocytes have RIPs associated with their surfaces, and wherein at least 25% of the modified lymphocytes in the cell formulation contain recombinant viral reverse transcriptase or integrase.

19. The cell formulation of claim 1, or the use of claim 2, wherein the polynucleotide further encodes a lymphoproliferative element.

20. The cell formulation of claim 1, or the use of claim 2, wherein the RIPs further comprise a binding polypeptide and a fusogenic polypeptide on the surface of the RIPs, wherein the binding polypeptide is capable of binding to a T cell, and wherein the fusogenic polypeptide is capable of mediating fusion of a RIP membrane with a membrane of a T cell.

21. The cell formulation of claim 1, or the use of claim 2, wherein the polynucleotide comprises one or more transcriptional units, wherein each of the one or more transcriptional units is operatively linked to a T cell-specific promoter.

22. The use of claim 10, wherein between 20 ml and 50 ml of blood is collected from the subject.

23. The use of claim 22, wherein the contacting is performed in a volume of between 20 ml and 50 ml.

24. The use of claim 22, wherein the blood cells are in whole blood during the contacting.

25. The use of claim 24, wherein the reaction mixture is applied to a leukoreduction filter after the contacting to remove at least 75% of RIPs not associated with lymphocytes from the reaction mixture after the contacting and before the formulating.

26. The use of claim 25, wherein at least 80% of the RIPs not associated with the lymphocytes are removed from the reaction mixture.

27. The use of claim 25, wherein the leukoreduction filter has an effective filtration area of between 3 cm² and 5 cm².

28. The use of claim 2, wherein the administering is for treating cancer in the subject.

29. The use of claim 2, wherein the administering is for treating a solid tumor in the subject.

30. The use of claim 29, wherein the solid tumor is a HER2 positive solid tumor.

31. The use of claim 29, wherein the subject experiences at least a partial response within 60 days after said administering.

32. The use of claim 31, wherein the cell formulation is only administered once to the subject before the partial response occurs.

33. The use of claim 2, wherein the RIPs do not comprise the CAR on their surface.

34. The cell formulation of claim 1, or the use of claim 2, wherein the one or more transcriptional units encode a second polypeptide comprising a lymphoproliferative element comprising an intracellular signaling domain from a cytokine receptor.

35. The use of claim 2, wherein the modified lymphocytes are administered subcutaneously in the presence of hyaluronidase.

36. The use of claim 11, wherein the modified lymphocytes are introduced back into the subject within 12 hours from the time the blood comprising the lymphocytes is collected from the subject.

37. The cell formulation of claim 1, or the use of claim 2, wherein the cell formulation comprises between 1×10⁶ and 1×10⁸ modified lymphocytes.

38. The use of claim 2, wherein the reaction mixture comprises at least 25% unfractionated whole blood by volume.

39. The use of claim 2, wherein the reaction mixture is in a closed cell processing system, wherein the contacting occurs when the reaction mixture is in a leukoreduction filter assembly in the closed cell processing system, and wherein the blood cells in the cell formulation are total nucleated cells (TNCs).

40. The use of claim 2, wherein the T cell activation element is on the surface of the RIPs. 7

41. The use of claim 2, wherein said T cell activation element comprises one or more of an antibody or an antibody mimetic capable of binding CD3, TCRα/β, CD28, or a mitogenic tetraspanin, or wherein said T cell activation element is a mitogenic tetraspanin.

42. The use of claim 41, wherein the T cell activation element comprises the antibody or the antibody mimetic capable of binding CD3, and wherein the T cell activation element is bound to the membrane of the RIPs.

43. The use of claim 42, wherein the membrane-bound anti-CD3 antibody or anti-CD3 antibody mimetic is an anti-CD3 scFv, an anti-CD3 scFvFc, or an anti-CD3 DARPin.

44. The use of claim 43, wherein the anti-CD3 antibody or anti-CD3 antibody mimetic is bound to the membrane by a GPI anchor, wherein the anti-CD3 antibody or anti-CD3 antibody mimetic is a recombinant fusion protein with a MuLV viral envelope protein, with or without a mutation at a furin cleavage site, or wherein the anti-CD3 antibody or anti-CD3 antibody mimetic is a recombinant fusion protein with a VSV viral envelope protein, or wherein the anti-CD3 antibody or anti-CD3 antibody mimetic is a recombinant fusion protein with a Henipavirus-G envelope protein.

45. The use of claim 2, wherein sufficient dimming units of the RIP are present to increase the percentage of CD8+ cells that are surface CD3−, to at least 50%.

46. The use of claim 2, wherein the reaction mixture is in a blood bag during the contacting.

47. The use of claim 2, wherein the reaction mixture is in contact with a leukoreduction filter assembly in a closed cell processing system after the contacting.

48. The cell formulation of claim 1, or the use of claim 2, wherein the CAR is an MRB-CAR.

49. The cell formulation or use of claim 21, wherein the promoter operatively linked to a first transcriptional unit is constitutively active, and wherein the RIPs further comprise a second transcriptional unit operatively linked to an inducible promoter, inducible in at least one of a T cell or an NK cell, wherein the first transcriptional unit and the second transcriptional unit are arranged in opposite directions, and wherein the second transcriptional units encodes a lymphoproliferative element.

50. The cell formulation of claim 1, or the use of claim 2, wherein the RIPs are lentiviral particles.

51. The cell formulation of claim 1, or the use of claim 2, wherein:
a) at least 25% of the modified lymphocytes in the cell formulation do not express one or more of the CAR or a transposase;
b) at least 25%, or optionally at least 50%, of the modified lymphocytes in the cell formulation comprise a recombinant viral reverse transcriptase or a recombinant viral integrase;
c) at least 25% of the modified lymphocytes in the cell formulation do not have the polynucleotide stably integrated into their genomes;
d) between 1% and 20%, of the modified lymphocytes in the cell formulation are genetically modified; and/or
e) at least 25% of the modified lymphocytes in the cell formulation are viable.

52. The cell formulation of claim 1, or the use of claim 2, wherein at least 5% of the modified lymphocytes in the cell formulation are genetically modified.

53. The use of claim 2, wherein the subject is a lymphoreplete subject.

54. The use of claim 2, wherein a second formulation is administered to the subject, wherein the second formulation comprises i) a cytokine, ii) an antibody, antibody mimetic, or polypeptide that is capable of binding CD3, CD28, OX40, 4-1BB, ICOS, CD9, CD53, CD63, CD81, and/or CD82, and/or iii) a source of the cognate antigen recognized by the CAR.

55. The cell formulation of claim 1, or the use of claim 2, wherein the cell formulation comprises a source of a cognate antigen for the CAR, wherein the source of the cognate antigen is the cognate antigen, an mRNA encoding the cognate antigen, or a cell expressing the cognate antigen.

56. The cell formulation of claim 1, or the use of claim 2, wherein the cell formulation comprises a cytokine and wherein the cytokine is IL-2, IL-7, IL-15, or IL-21 or a modified version of any of these cytokines that is capable of binding to and activating a native receptor for the cytokine.

57. The use of claim 2, wherein:
i) the reaction mixture comprises at least 25% unfractionated whole blood by volume,
ii) the reaction mixture comprises neutrophils, and/or
iii) the modified T cells and/or NK cells are administered subcutaneously in a delivery solution comprising neutrophils.

58. A population of genetically modified lymphocytes, comprising:
at least 1×10⁴ genetically modified lymphocytes expressing a chimeric antigen receptor (CAR), wherein at least some of the genetically modified lymphocytes are localized subcutaneously in a subject, and wherein the genetically modified lymphocytes comprise T cells.

59. The population of genetically modified lymphocytes of claim 58, wherein the population further comprises other white blood cells that do not express the CAR.

60. The population of genetically modified lymphocytes of claim 59, wherein the population comprises one or more aggregates of at least 20, cells each.

61. A subcutaneous lymphoid structure, comprising at least some of the modified lymphocytes of the population of genetically modified lymphocytes of claim 59.

62. The subcutaneous lymphoid structure of claim 61, or the population of genetically modified lymphocytes of claim 58, wherein some of the genetically modified lymphocytes expressing the CAR are located in lymphatic vasculature.

63. The population of genetically modified lymphocytes of claim 58, wherein the other white blood cells comprise B cells, macrophages, dendritic cells, T cells and/or NK cells.

64. The subcutaneous lymphoid structure of claim 61, wherein some of the modified lymphocytes of the population are in lymphatic vasculature localized within 25, 50, 75, 100, 125, 150, 200, 250, 500, or 1,000 μm from, the subcutaneous lymphoid structure.

65. The subcutaneous lymphoid structure of claim 61, or the population of genetically modified lymphocytes of claim 58, further comprising actively dividing lymphocytes that are native to the subject and do not express the CAR.

66. The subcutaneous lymphoid structure of claim 61, or the population of genetically modified lymphocytes of claim 58, wherein the genetically modified lymphocytes express a lymphoproliferative element.

67. The population of genetically modified lymphocytes of claim 58, wherein the population of genetically modified lymphocytes is in an artificial lymph node.

68. The population of genetically modified lymphocytes of claim 58, wherein the T cells comprise CD4+ and CD8+ cells, and wherein at least 50% of the genetically modified lymphocytes that are CD4+ and/or CD8+ are surface CD3−.

69. The population of genetically modified lymphocytes of claim 58, wherein:
i) the population of genetically modified lymphocytes comprises a persisting population of genetically modified lymphocytes expressing the chimeric antigen receptor (CAR), that persists in the subject for at least 21 after administration.
ii) the genetically modified lymphocytes produce a population of progeny lymphocytes, wherein the population of progeny lymphocytes comprises at least 1×10⁵ cells;
iii) the population of genetically modified lymphocytes comprises at least 100 genetically modified lymphocytes localized subcutaneously and the subcutaneous region contains no artificial matrix components; and/or
iv) the at least 10 genetically modified lymphocytes of the population remain localized subcutaneously for at least 14 days.

70. The population of genetically modified lymphocytes of claim 58, wherein at least 1×10⁵ genetically modified lymphocytes are located subcutaneously and wherein at least 1×10⁵ genetically modified lymphocytes are circulating in the blood and/or at the site of a tumor in the subject.

71. A leukoreduction filtration assembly, comprising
a) a reaction mixture collection container with a maximum volume of 100 ml;
b) a leukoreduction filter; and
c) a collection valve,
d) wherein an inlet channel connects a first assembly opening to a leukoreduction filter enclosure comprising the leukoreduction filter, wherein a first connection junction between the first assembly opening and the inlet channel has an angle of between 5° and 60° with respect to the inlet channel, and the inlet channel has no junctions that are greater than 80, and wherein the leukoreduction filter has an effective filtration area of between 2 cm² and 5 cm².

72. A method for genetically modifying mammalian nucleated blood cells, comprising:
a) transporting between 10 ml and 50 ml of whole blood comprising whole blood cells into a transduction assembly comprising an incubation bag comprising nucleic acid vector copies to form a reaction mixture, wherein the incubation bag has a maximum volume capacity of 75 ml, and wherein the incubation bag is connected to an input channel at a first assembly opening;
b) contacting the whole blood cells with the vector copies within the reaction mixture to produce modified whole blood cells;
c) transporting the modified whole blood cells through a channel to a reaction mixture collection container;
d) transporting the modified whole blood cells from the reaction mixture collection container to a leukoreduction filter of a leukoreduction filter assembly to filter the modified whole blood cells to produce an enriched fraction of modified nucleated blood cells, wherein the leukoreduction filter has effective filtration area of between 3 cm² and 5 cm²; and
e) collecting between the enriched fraction of modified blood cells in 1 ml to 20 ml a delivery solution to form a cell formulation comprising a suspension of the modified nucleated blood cells, wherein at least 10% of the modified cells in the cell formulation are aggregated.

73. Use of replication incompetent recombinant retroviral particles in the manufacture of a kit for modifying and/or genetically modifying T cells and/or NK cells subcutaneously in a subject, wherein the use of the kit comprises:
administering to the subject subcutaneously, a modifying composition comprising replication incompetent recombinant retroviral particles (RIPs) and an activation element, wherein the RIPs comprise a polynucleotide encoding a first polypeptide comprising a transgene, an antigen, an engineered T cell receptor, or a chimeric antigen receptor (CAR),
wherein the modifying composition has a volume between 0.5 ml and 10 ml contained within a syringe, wherein said administering facilitates association of the T cells and/or NK cells with the RIPs, wherein the T cells and/or NK cells are present in the subcutaneous region of the subject, and wherein the RIPs modify the T cells and/or NK cells to form a population of modified T cells and/or NK cells the modifying composition.

74. The use of claim 73, further comprising administering a cell suspension to the subject subcutaneously, wherein the administering the cell suspension has a volume between 2 ml and 25 ml contained within a syringe, wherein the cell suspension comprises T cells and/or NK cells, wherein the RIPs in the modifying composition contact the T cells and/or NK cells, thereby modifying and/or genetically modifying the T cells and/or NK cells in the cell suspension.

75. A method for determining the amount of a preparation of a gene vector encapsulated in a membrane (i.e. gene vector particle) to add to a target cell suspension, comprising:
determining the dimming units of the gene vector under dimming conditions comprising a reaction mixture, wherein gene vector particles of the preparation express a binding polypeptide on their surface, and wherein dimming units are the amount or volume of the gene vector that reduces a target surface polypeptide by a target percentage in a target volume of a control cell suspension expressing the surface polypeptide, or a same on-test blood preparation to which the gene vector will be contacted, or as compared to another surface marker in a target cell population that expresses the surface polypeptide, under contacting conditions.

76. The method of claim 75, wherein the amount of the gene vector (e.g. viral particle) preparation to add is determined by the dimming units of the gene vector (e.g. viral particle) preparation, the target dimming percent of the surface polypeptide on the target cell (e.g. T cell) suspension, and an approximate, estimated, calculated and/or empirically-determined concentration of the surface polypeptide on the T cell suspension.

77. A kit for modifying NK cells and/or T cells, comprising:
one or a plurality of containers,
wherein at least one of the plurality of containers comprises either i) polynucleotides, each encoding a first polypeptide comprising an engineered T cell receptor or a chimeric antigen receptor (CAR), or ii) T cells or NK cells each capable of expressing the CAR,
wherein at least one of the plurality of containers contains one or more additional components selected from: a composition comprising i) a cytokine, ii) a source of the cognate antigen recognized by the CAR, and iii) a target cell depletion agent, and
wherein at least one of the plurality of containers contain a delivery solution adapted for subcutaneous administration, and/or wherein the kit further comprises one or more sterile syringes adapted for subcutaneous delivery of T cells and/or NK cells.

78. The kit of claim 77, wherein the kit further comprises a leukoreduction filter assembly.

79. A kit for modifying NK cells and/or T cells, comprising:
one or a plurality of containers,
wherein at least one of the plurality of containers contains polynucleotides comprising a first transcriptional unit operatively linked to a promoter active in T cells and/or NK cells, wherein the first transcriptional unit encodes a first polypeptide comprising a chimeric antigen receptor (CAR), and
wherein at least one of the plurality of containers contains one or more additional components selected from: a composition comprising i) a cytokine and ii) a source of the cognate antigen recognized by the CAR, or iii) a binding partner for an external epitope of the CAR; and wherein at least one of the plurality of containers contain a delivery solution adapted for subcutaneous administration, and/or wherein the kit further comprises one or more sterile syringes adapted for subcutaneous delivery of T cells and/or NK cells.

80. A subcutaneous reaction mixture, comprising:
i) modified lymphocytes having one or more gene vectors, for example replication incompetent recombinant retroviral particles (RIPs) associated with their surfaces, wherein the modified lymphocytes comprise T cells and/or NK cells, wherein the T cells comprise CD4+ cells and CD8+ cells, and wherein the NK cells comprise CD56+ cells,
wherein the gene vectors or replication incompetent recombinant retroviral particles comprise a polynucleotide encoding a transgene, an antigen, an engineered T cell receptor, a chimeric antigen receptor (CAR),
wherein the gene vectors or replication incompetent recombinant retroviral particles comprise a polypeptide capable of binding to a surface polypeptide, a T cell receptor complex polypeptide, or CD3, associated with the surface of the gene vectors or replication incompetent recombinant retroviral particles, and
ii) a cytokine, ii) an antibody, antibody mimetic, or polypeptide that is capable of binding CD3, CD28, OX40, 4-1BB, ICOS, CD9, CD53, CD63, CD81, and/or CD82, and/or iii) a source of the cognate antigen recognized by the CAR.
